(19) United States
(12) Patent Application Publication
(10) Pub. No.: US 2025/0312473 A1
(43) Pub. Date: Oct. 9, 2025

(54) B7H3 ANTIBODY DRUG CONJUGATES

(71) Applicant: BEONE MEDICINES I GMBH, Basel (CH)

(72) Inventors: Charng-Sheng Tsai, Beijing (CN); Mei-Hsuan Tsai, Shanghai (CN); Maomao He, Shanghai (CN); Zewei Wang, Shanghai (CN); Xiaodong Wei, Shanghai (CN); Wei LUO, Shanghai (CN); Xiaokun Yang, Shanghai (CN); Xiaoyan Tang, Beijing (CN); Qi Liu, Beijing (CN); Liu Xue, Beijing (CN)

(73) Assignee: BEONE MEDICINES I GMBH, Basel (CH)

(21) Appl. No.: 19/244,134

(22) Filed: Jun. 20, 2025

Related U.S. Application Data
(63) Continuation of application No. PCT/CN2023/142853, filed on Dec. 28, 2023.

(30) Foreign Application Priority Data
Dec. 29, 2022 (WO) PCT/CN2022/143246
Dec. 29, 2022 (WO) PCT/CN2022/143247
Dec. 29, 2022 (WO) PCT/CN2022/143248

(51) Int. Cl. A61K 47/68; A61K 45/06; A61P 35/00; C07K 16/28

(52) U.S. Cl. CPC A61K 47/68037; A61K 45/06; A61K 47/6849; A61K 47/6889; A61P 35/00; C07K 16/2827

(57) ABSTRACT

Provided herein are antibody drug conjugates comprising an antibody or antigen binding fragment thereof capable of specific binding to human B7H3 and a cytotoxic agent, such as an exatecan analogue. Also provided are pharmaceutical compositions comprising the antibody drug conjugates and methods of using the antibody drug conjugates, e.g., to treat a 4Ig-B7H3 positive cancer.

### I. CROSS-REFERENCE TO RELATED APPLICATIONS

This application is a continuation of International Application No. PCT/CN2023/142853, filed Dec. 28, 2023, entitled “B7H3 Antibody Drug Conjugates,” which claims the benefit of priority of PCT Application No. PCT/CN2022/143248, filed Dec. 29, 2022, entitled “Anti-B7H3 Antibodies and Methods of Use,” and PCT/CN2022/143247, filed Dec. 29, 2022, entitled “Anti-B7H3 Antibodies and Methods of Use,” and PCT/CN2022/143246, filed Dec. 29, 2022, entitled “B7H3 Antibody Drug Conjugates,” each of which is hereby incorporated by reference in its entirety.

### 2. REFERENCE TO ELECTRONIC SEQUENCE LISTING

The application contains a Sequence Listing which has been submitted electronically in .XML format and is hereby incorporated by reference in its entirety. Said .XML copy, created on Dec. 27, 2023, is named “01368-0060-00PCT.xml” and is 446,058 bytes in size. The sequence listing contained in this .XML file is part of the specification and is hereby incorporated by reference herein in its entirety.

### 3. FIELD

The present disclosure generally relates to anti-B7H3 antibody drug conjugates, and their uses for the treatment of cancer.

### 4. BACKGROUND

The B7 homology 3 protein (B7H3) (also known as CD276, B7-H3, and B7RP-2, and referred to herein as “B7H1-3”) is a type I transmembrane glycoprotein of the immunoglobulin superfamily. Human B7H3 contains a putative signal peptide, V-like and C-like Ig domains, a transmembrane region and a cytoplasmic domain. Exon duplication in humans results in the expression of two B7H13 isoforms having either a single IgV-IgC-like domain (2IgB7H3 isoform) or a IgV-IgC-IgV-IgC-like domain (4IgB7H3 isoform) containing several conserved cysteine residues. The predominant B7H3 isoform in human tissues and cell lines is the 4IgB7H3 isoform (Steinberger et al., J. Immunol. 172(4): 2352-9 (2004)).

B7H3 has been reported as having both co-stimulatory and co-inhibitory signaling functions (see, e.g., Chapoval et al., Nat. Immunol. 2: 269-74 (2001); Suh et al., Nat. Immunol. 4: 899-906 (2003); Prasad et al., J. Immunol. 173: 2500-6 (2004); and Wang et al., Eur. J. Immunol. 35: 428-38 (2005)). As an example of the co-stimulatory function of B7H3, in vitro studies have shown B7H3 was able to increase proliferation of cytotoxic T-lymphocytes (CTLs) and upregulate interferon gamma (IFN-7) production in the presence of anti-CD3 antibody to mimic the T cell receptor signal (Chapoval et al., 2001). Moreover, in vivo studies using cardiac allografts in B7H3−/− mice showed decreased production of key cytokine, chemokine, and chemokine receptor mRNA transcripts (e.g., IL-2, IFN-γ, monocyte chemoattractant protein (MCP-1) and IFN-inducible protein (IP)-10) as compared to wild-type control (Wang et al., 2005). In contrast, the co-inhibitory function of B7H3 has been observed, for example, in mice, where B7H3 protein inhibited T-cell activation and effector cytokine production (Suh et al., 2003). Although no ligands have been identified for human B7H3, murine B7H3 has been found to bind to the triggering receptor expressed on myeloid cells (TREM-) like transcript 2 (TLT-2), a modulator of adaptive and innate immunity cellular responses. Binding of murine B7H3 to TLT-2 on CD8+ T-cells enhances T-cell effector functions such as proliferation, cytotoxicity. and cytokine production (Hashiguchi et al., Proc. Nat'l. Acad. Sci. U.S.A. 105(30): 10495-500 (2008)).

B7H3 is not constitutively expressed in many immune cells (e.g., natural killer (NK) cells, T-cells, and antigen-presenting cells (APCs)), however, its expression can be induced. Further, the expression of B7H3 is not restricted to immune cells. B7H3 transcripts are expressed in a variety of human tissues including colon, heart, liver, placenta, prostate, small intestine, testis, and uterus, as well as osteoblasts, fibroblasts, epithelial cells, and other cells of non-lymphoid lineage, potentially indicating immunological and non-immunological functions (Nygren et al. Front Biosci. 3:989-93 (2011)). However, protein expression in normal tissue is typically maintained at a low level and thus, may be subject to post-transcriptional regulation.

B7H3 is also expressed in a variety of human cancers, including prostate cancer, clear cell renal cell carcinoma, glioma, melanoma, lung cancer, non-small cell lung cancer (NSCLC), small cell lung cancer, pancreatic cancer, gastric cancer, acute myeloid leukemia (AMIL), non-Hodgkin's lymphoma (NHL), ovarian cancer, colorectal cancer, colon cancer, renal cancer, hepatocellular carcinoma, kidney cancer, head and neck cancer, hypopharyngeal squamous cell carcinoma, glioblastoma, neuroblastoma, breast cancer, endometrial cancer, and urothelial cell carcinoma. Although the role of 1371-13 in cancer cells is unclear, its expression may orchestrate signaling events that may protect cancer cells from innate and adaptive immune responses. For example, 137113 is overexpressed in high-grade prostatic intraepithelial neoplasia and adenocarcinomas of the prostate, and high expression levels of B7H3 in these cancerous cells are associated with an increased risk of cancer progression after surgery (Roth et al. Cancer Res. 67(16): 7893-900 (2007)). Further, tumor B7H3 expression in NSCLC inversely correlated with the number of tumor-infiltrating lymphocytes and significantly correlated with lymph node metastasis (Sun et al. Lung Cancer 53(2): 143-51 (2006)). The level of circulating soluble B7H3 in NSCLC patients has also been associated with higher tumor stage, tumor size, lymph node metastasis, and distant metastasis (Yamato et al., Br. J. Cancer 101(10):1709-16 (2009)).

B7H3 may also play an important role in T-cell-mediated antitumor responses in a context dependent manner. For example, gastric cancer tumor cell expression of B7H3 positively correlated with survival time, infiltration depth, and tissue type (Wu et al., World J. Gastroenterol. 12(3): 457-9 (2006)). Further, high expression of B7H3 in pancreatic tumor cells was associated with patient survival after surgical resection and significantly correlated with the number of tumor-infiltrating CD8+ T-cells (Loos et al., BMC Cancer 9:463 (2009)).

Antibody drug conjugates (ADC) represent a relatively new class of therapeutics comprising an antibody conjugated to a cytotoxic drug via a chemical linker. The therapeutic concept of ADCs is to combine binding capabilities of an antibody with a drug, where the antibody is used to deliver the drug to a tumor cell by means of binding to a target surface antigen, including target surface antigens that are overexpressed in tumor cells.

There remains a need in the art for anti-B7H3 ADCs that can be used for therapeutic purposes, e.g., in the treatment of 4Ig-B7H3 positive cancer.

### 5. BRIEF SUMMARY

Provided here is an antibody drug conjugate having Formula (I):

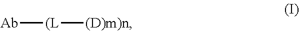

or a pharmaceutically acceptable salt thereof, wherein:
Ab is an antibody or antigen binding fragment thereof capable of specific binding to human
B7H3;
L is a linker;
D is a residue of a cytotoxic agent;
m is an integer from 1 to 8; and
n is from 1 to 10.

In one embodiment, m is 1.

In one embodiment, n is from 3 to 10, e.g., about 3, 4, 5, 6, 7, 8, 9, or 10. In one embodiment, n is about 8.

In one embodiment, the antibody drug conjugate has Formula (II):

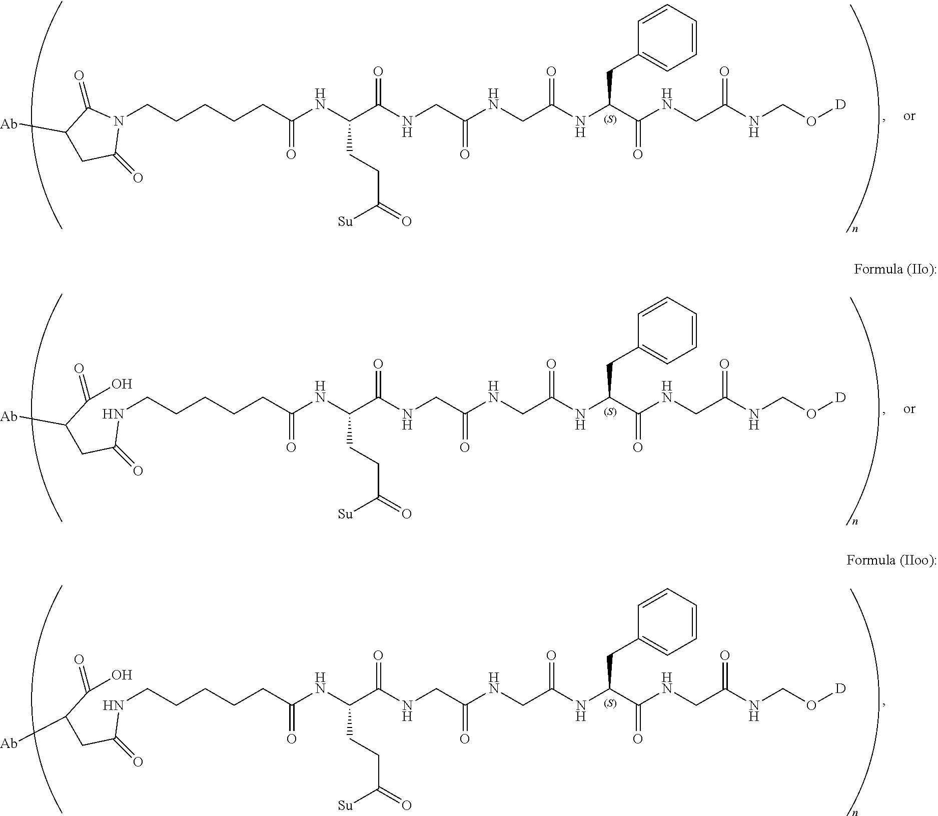

or a pharmaceutically acceptable salt thereof, wherein Su is a hydrophilic residue.

In one embodiment, Su is

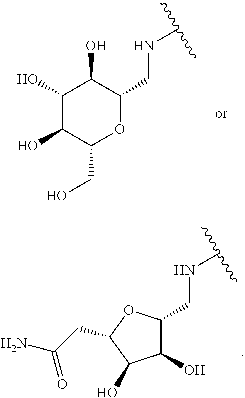 [SMILES: *NC[C@@H]1O[C@H](CO)[C@@H](O)[C@H](O)[C@H]1O ; *NC[C@H]1O[C@@H](CC(N)=O)[C@H](O)[C@@H]1O]

In one embodiment, the antibody drug conjugate comprises Formula (III):

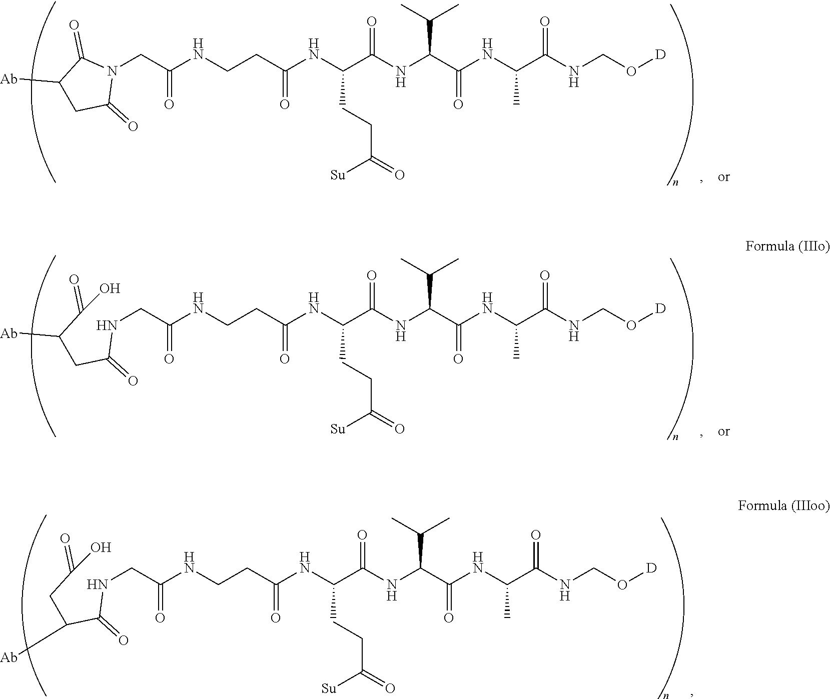

or a pharmaceutically acceptable salt thereof wherein Su is a hydrophilic residue.

In one embodiment, the antibody drug conjugate comprises Formula (III):

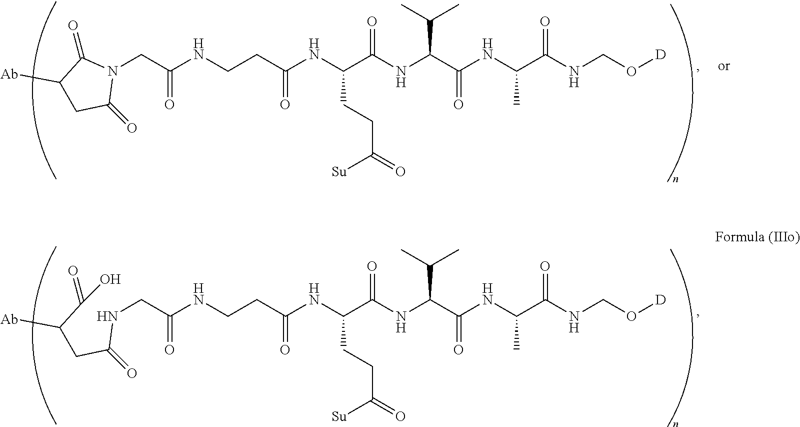

or a pharmaceutically acceptable salt thereof, wherein Su is a hydrophilic residue.

In one embodiment, Su is

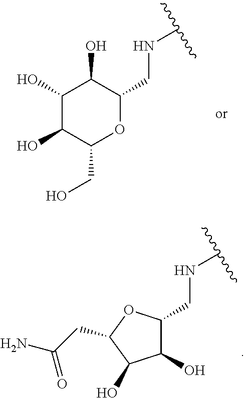 [SMILES: *NC[C@@H]1O[C@H](CO)[C@@H](O)[C@H](O)[C@H]1O ; *NC[C@H]1O[C@@H](CC(N)=O)[C@H](O)[C@@H]1O]

In one embodiment, D is

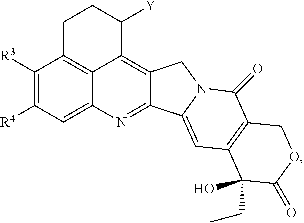

wherein
Y is -A-B-C′-D′-*, wherein * marks the bond where D connects to the antibody-drug conjugate;
A is a bond, CR¹R², or N—R¹;
B is a bond, —C(═O)—, or —C(═O)O—;
C′ is a bond, or a divalent group, wherein the divalent group is unsubstituted or substituted C₁₋₈ alkyl, unsubstituted or substituted cycloalkyl, unsubstituted or substituted heterocyclyl, unsubstituted or substituted aryl, or unsubstituted or substituted heteroaryl;
D′ is a bond, NH, or O;
each of R¹ and R² is, independently, hydrogen, halogen, substituted or unsubstituted alkyl, or substituted or unsubstituted alkoxyl; or R¹ and R² together with the atom to which they are attached form unsubstituted or substituted cycloalkyl, unsubstituted or substituted heterocyclyl, unsubstituted or substituted aryl, or unsubstituted or substituted heteroaryl;
each of R³ and R⁴ is, independently, hydrogen, halogen, substituted or unsubstituted alkyl, or substituted or unsubstituted alkoxyl; or R¹ and R⁴ together with the atoms to which they are attached form unsubstituted or substituted cycloalkyl, unsubstituted or substituted heterocyclyl, unsubstituted or substituted aryl, or unsubstituted or substituted heteroaryl.

In one embodiment, D is

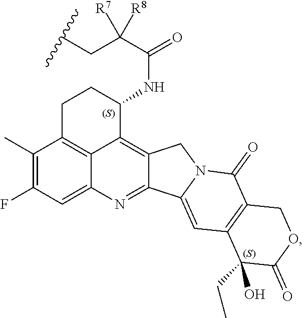

wherein R⁷ and R⁸ are each independently hydrogen, halogen, or alkyl.

In one embodiment, D is

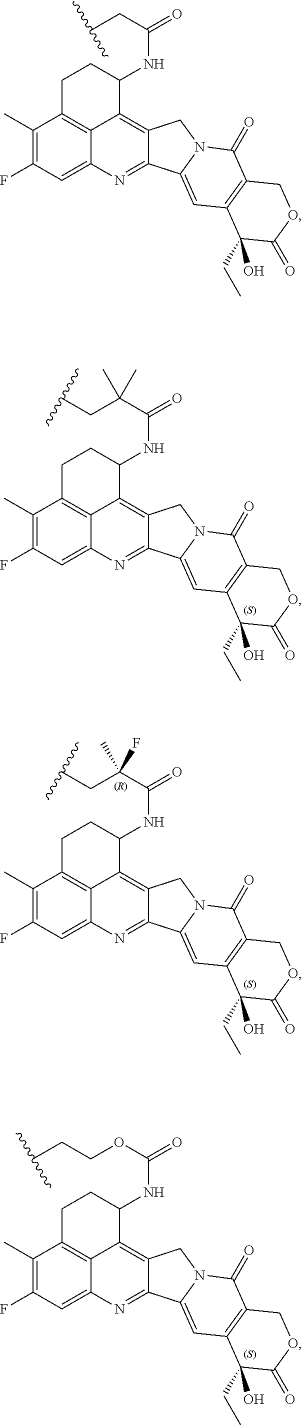

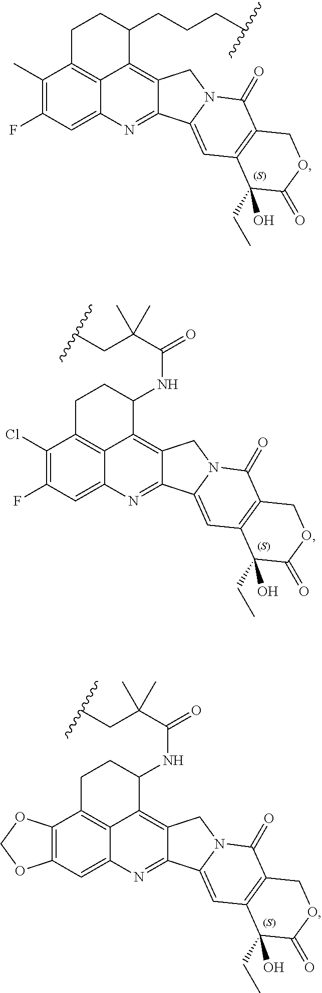

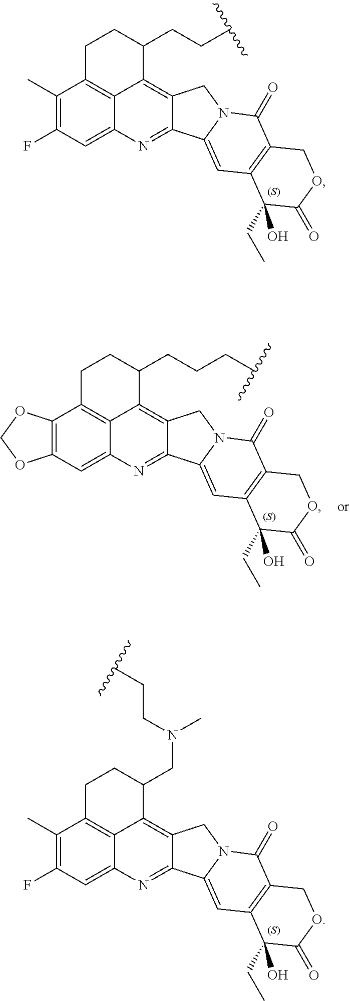

In one embodiment, D is

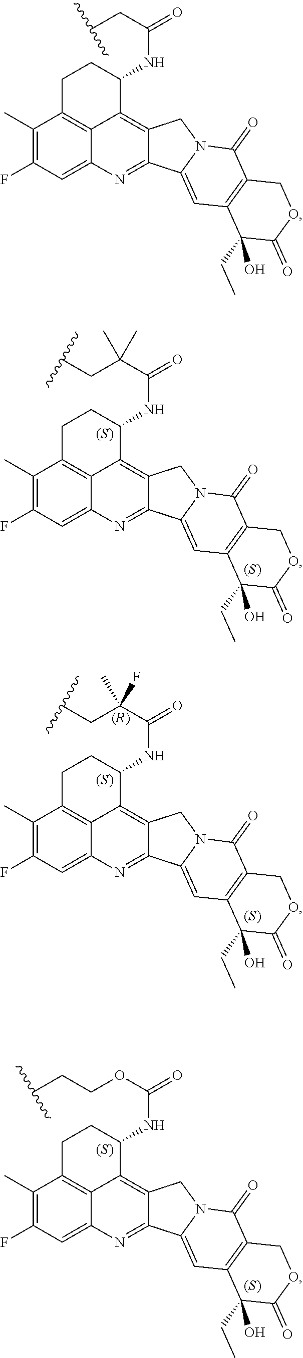

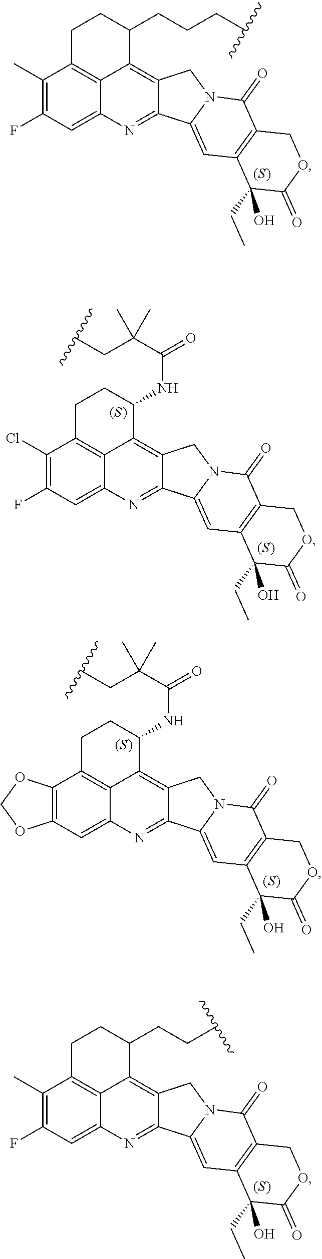

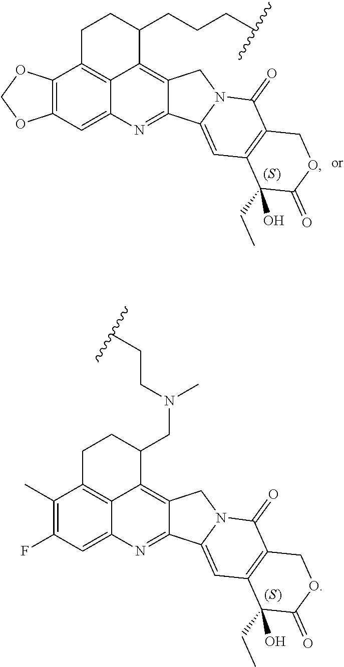

In one embodiment, the antibody drug conjugate has one of the following formulas, or a tautomer, stereoisomer, pharmaceutically acceptable salt, or solvate thereof:

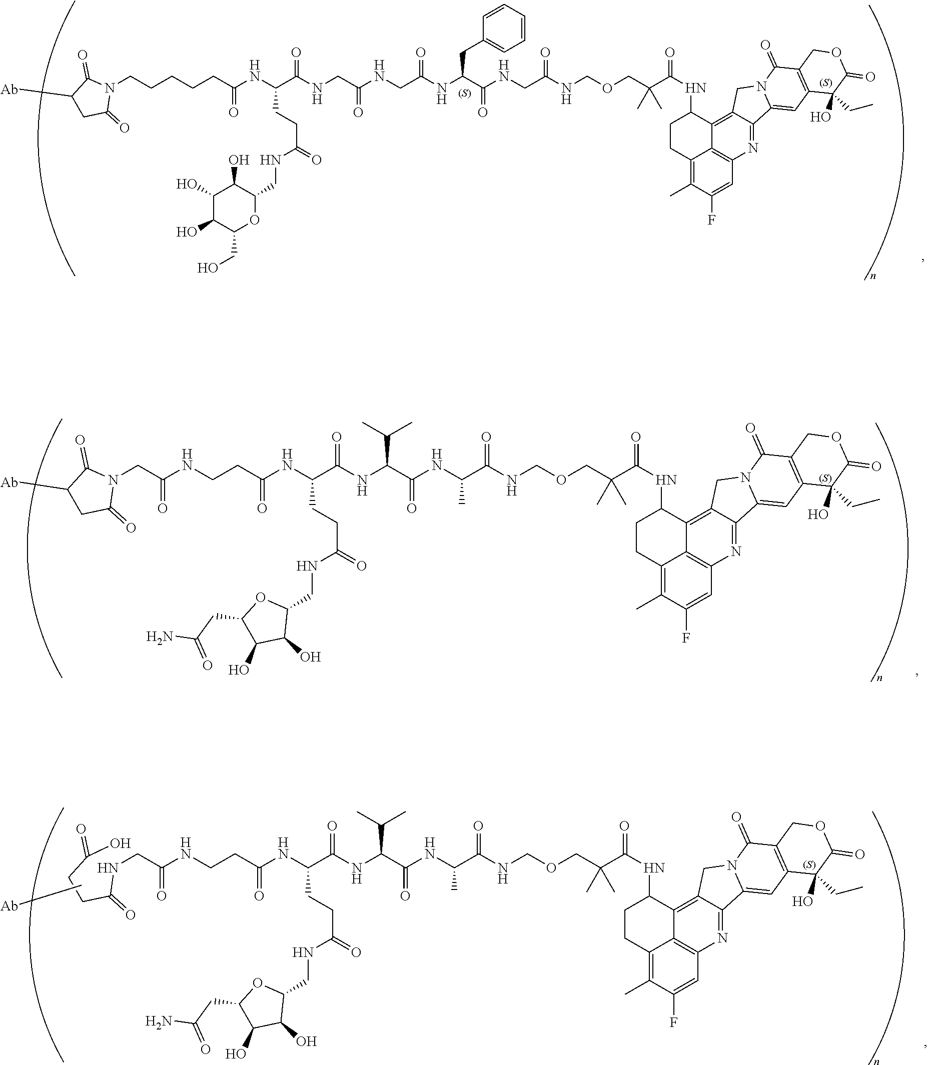

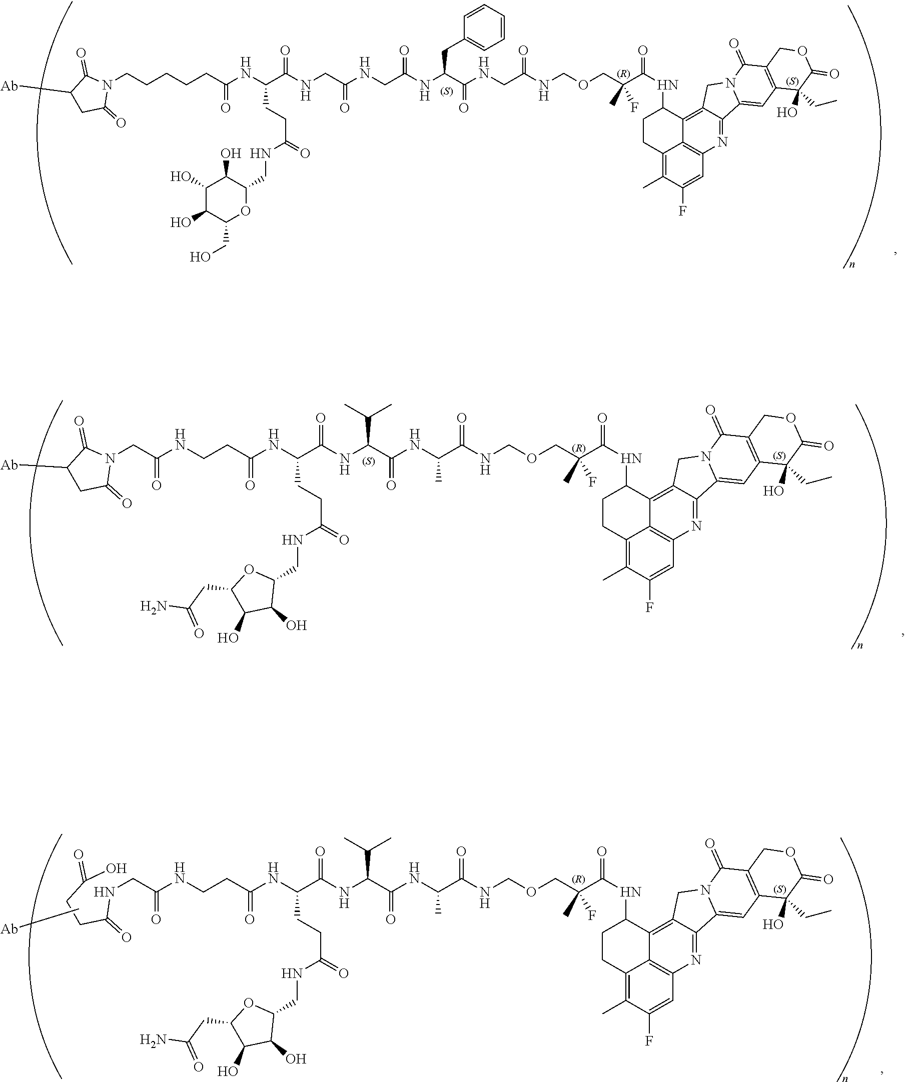

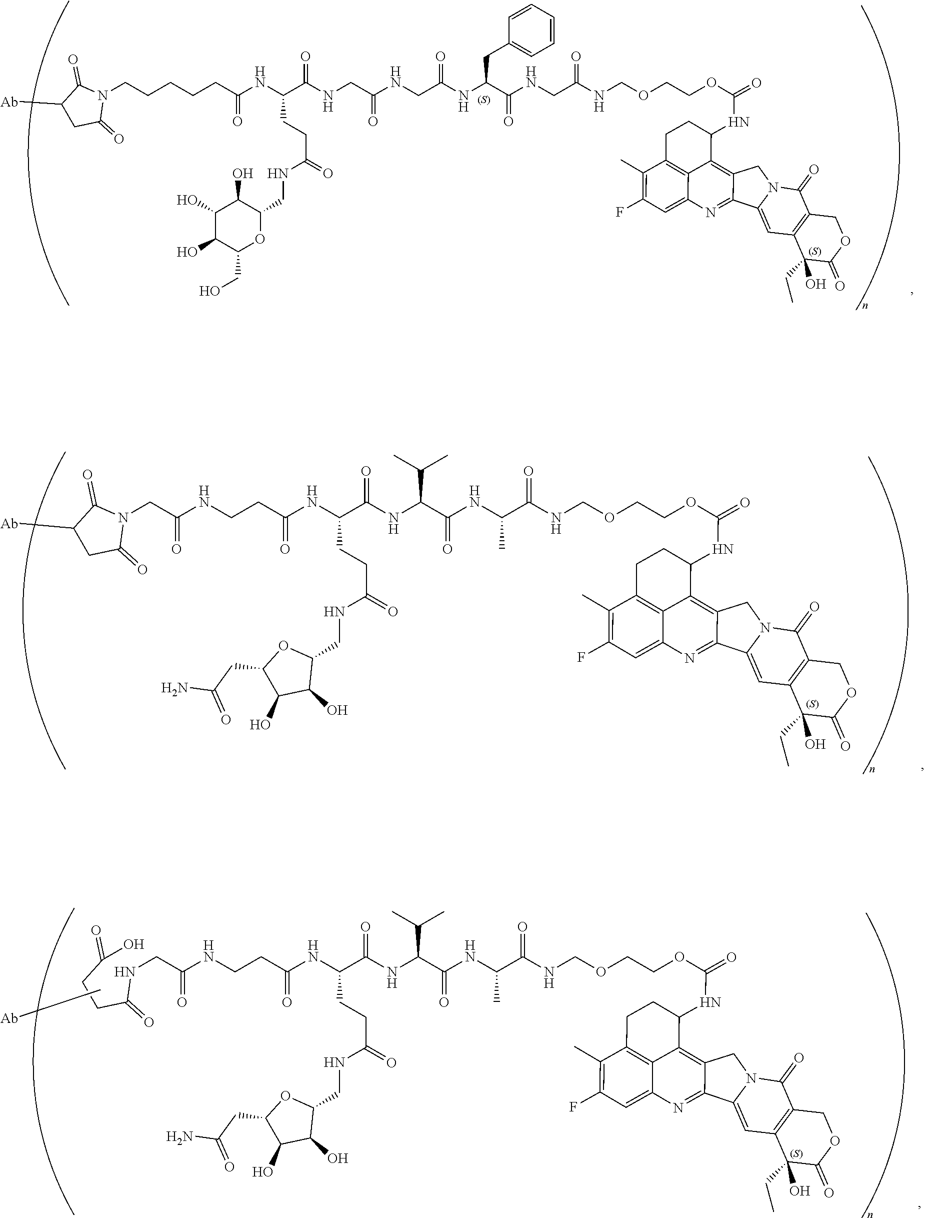

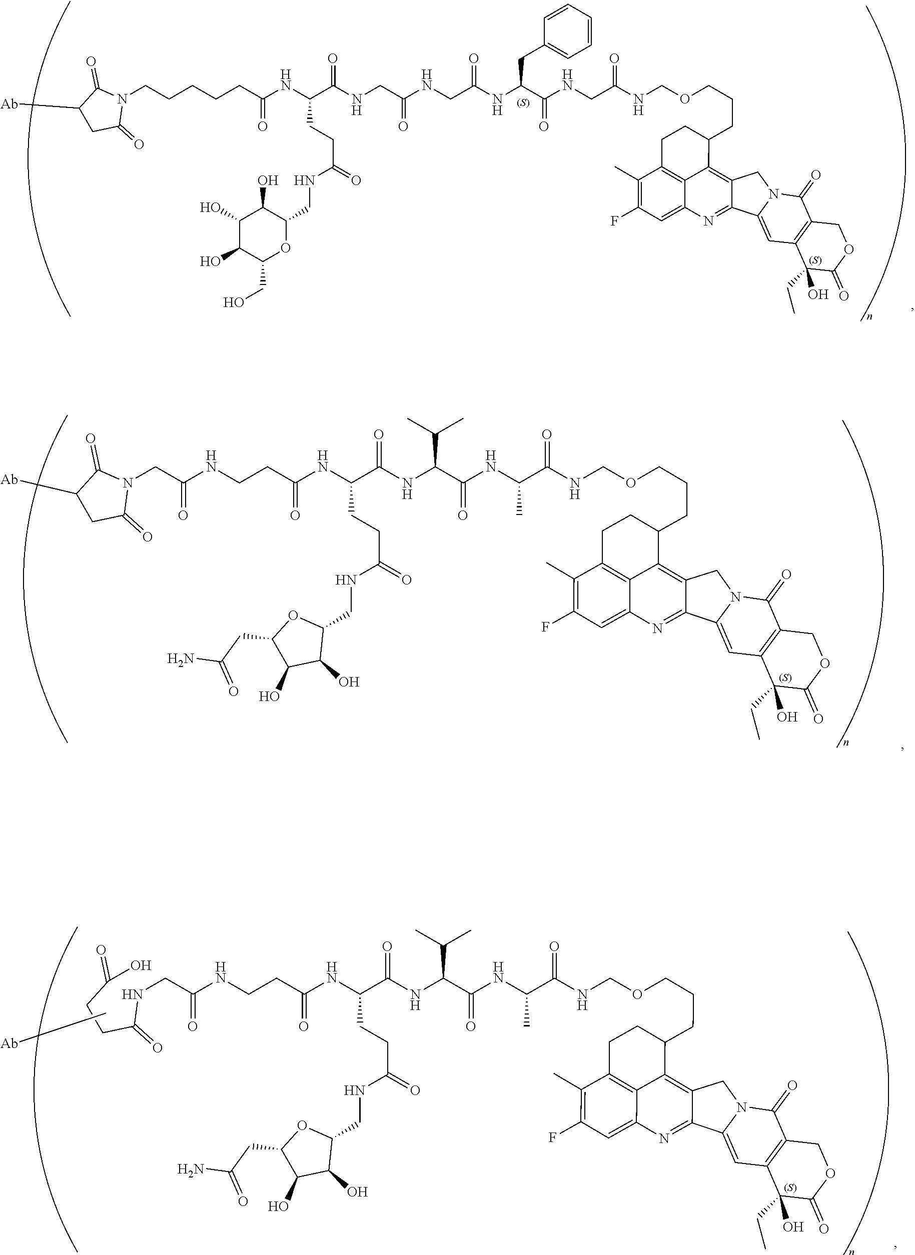

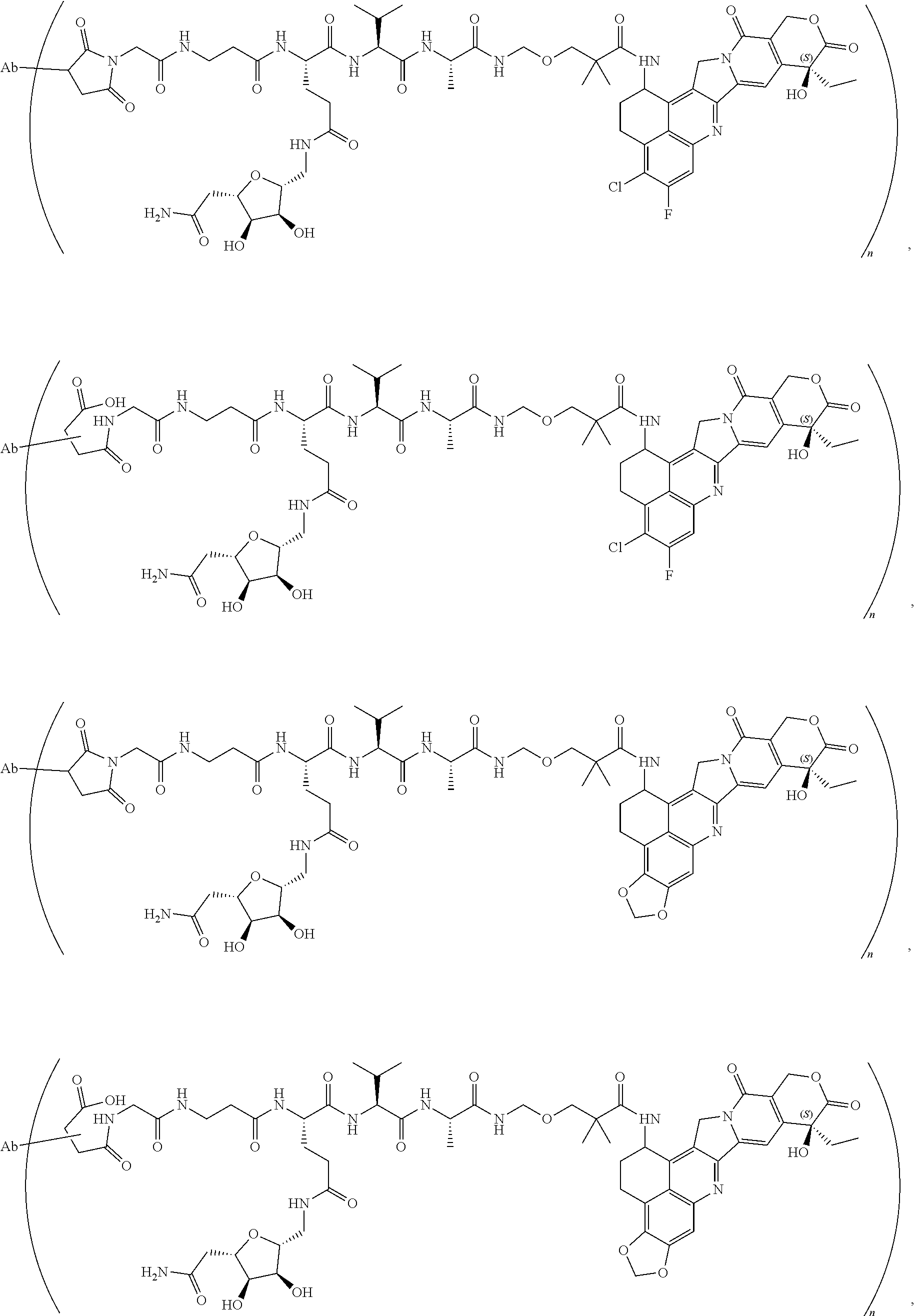

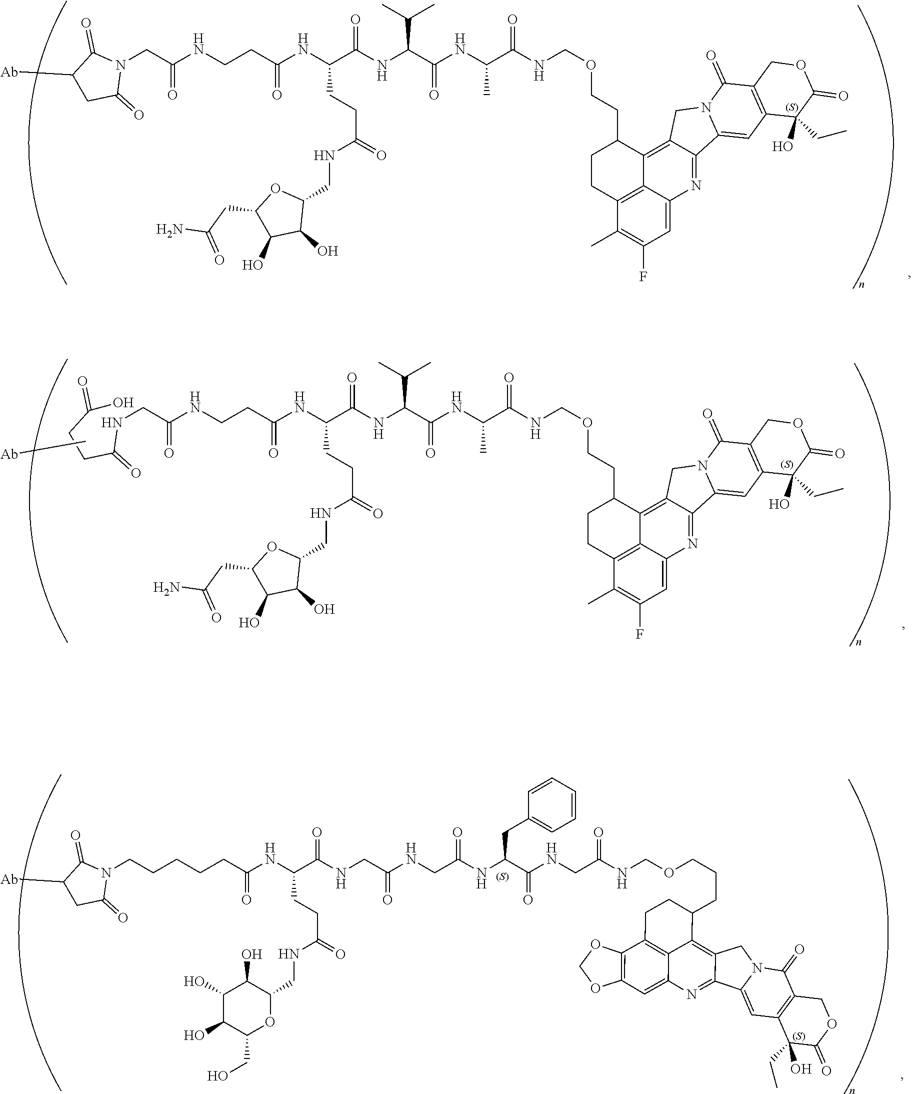

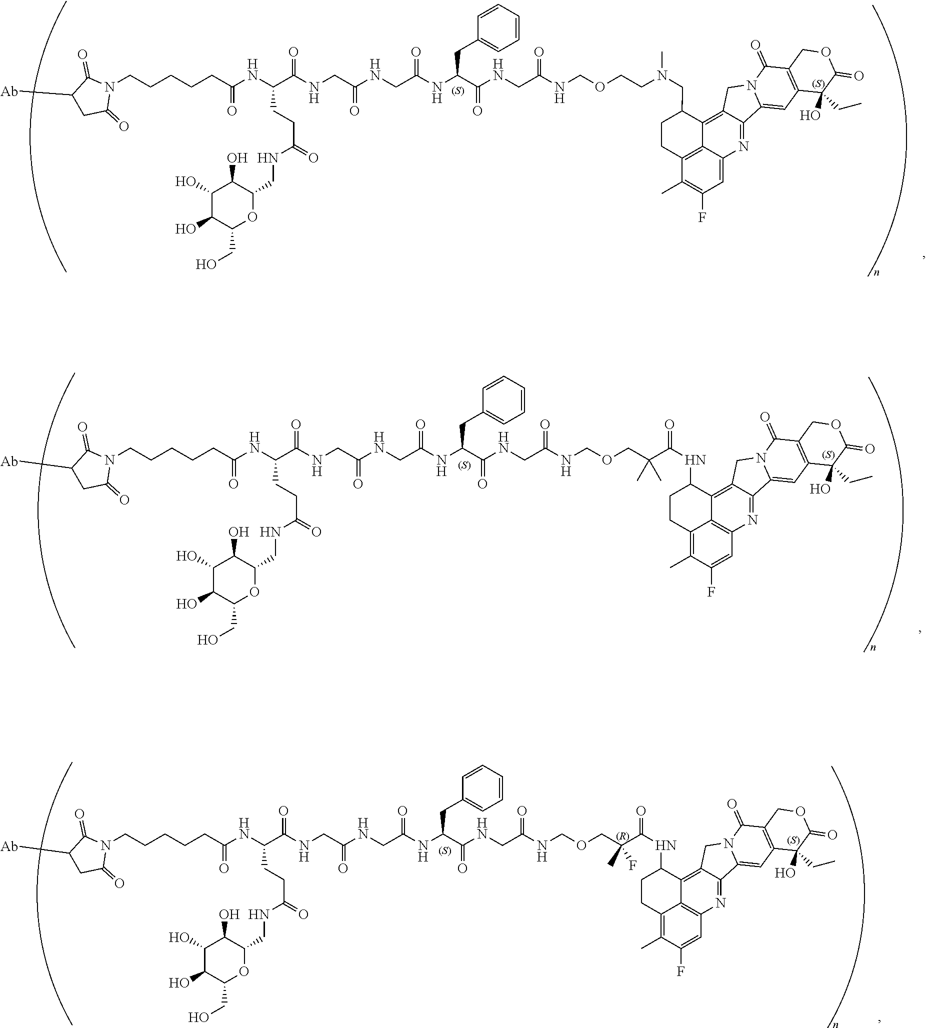

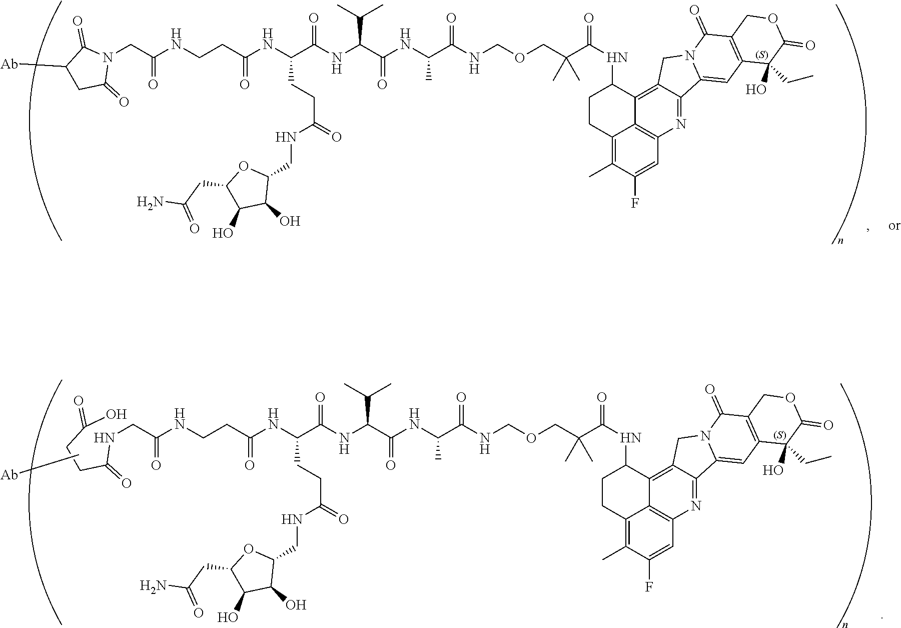

In one embodiment, the antibody or antigen-binding fragment comprises:
(i) a heavy chain variable region (VH) that comprises (a) a HCDR1 (Heavy Chain Complementarity Determining Region 1) of SEQ ID NO: 11, (b) a HCDR2 of SEQ ID NO: 2, and (c) a HCDR3 of SEQ ID NO: 14; and a light chain variable region (VL) that comprises (d) a LCDR1 (Light Chain Complementarity Determining Region 1) of SEQ ID NO: 23, (e) a LCDR2 of SEQ ID NO: 5, and (f) a LCDR3 of SEQ ID NO: 6;
(ii) a heavy chain variable region (VH) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises (d) a LCDR1 of SEQ ID NO: 4, (e) a LCDR2 of SEQ ID NO: 5, and (f) a LCDR3 of SEQ ID NO: 6;
(iii) a heavy chain variable region (VH) that comprises (a) a HCDR1 of SEQ ID NO: 11, (b) a HCDR2 of SEQ ID NO: 2, and (c) a HCDR3 of SEQ ID NO: 3; and a light chain variable region (VL) that comprises (d) a LCDR1 of SEQ ID NO: 4, (e) a LCDR2 of SEQ ID NO: 5, and (f) a LCDR3 of SEQ ID NO: 6;
(iv) a heavy chain variable region (VH) that comprises (a) a HCDR1 of SEQ ID NO: 1, (b) a HCDR2 of SEQ ID NO: 2, and (c) a HCDR3 of SEQ ID NO: 14; and a light chain variable region (VL) that comprises (d) a LCDR1 of SEQ ID NO: 4, (e) a LCDR2 of SEQ ID NO: 5, and (f) a LCDR3 of SEQ ID NO: 6;
(v) a heavy chain variable region (VII) that comprises (a) a HCDR1 of SEQ ID NO: 1, (b) a HCDR2 of SEQ ID NO: 2, and (c) a HCDR3 of SEQ ID NO: 17; and a light chain variable region (VL) that comprises (d) a LCDR1 of SEQ ID NO: 4, (e) a LCDR2 of SEQ ID NO: 5, and (f) a LCDR3 of SEQ ID NO: 6;
(vi) a heavy chain variable region (VI) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises (d) a LCDR1 of SEQ ID NO: 20, (e) a LCDR2 of SEQ ID NO: 5, and (f) a LCDR3 of SEQ ID NO: 6;
(vii) a heavy chain variable region (VH) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises (d) a LCDR1 of SEQ ID NO: 23, (e) a LCDR2 of SEQ ID NO: 5, and (f) a LCDR3 of SEQ ID NO: 6;
(viii) a heavy chain variable region (VH) that comprises (a) a HCDR1 of SEQ ID NO: 11, (b) a HCDR2 of SEQ ID NO: 2, and (c) a HCDR3 of SEQ ID NO: 28; and a light chain variable region (VL) that comprises: (d) a LCDR1 of SEQ ID NO: 23, (e) a LCDR2 of SEQ ID NO: 5, and (f) a LCDR3 of SEQ ID NO: 6; or
(ix) a heavy chain variable region (VH) that comprises (a) a HCDR1 of SEQ ID NO: 300, (b) a HCDR2 of SEQ ID NO: 1700, and (c) a HCDR3 of SEQ ID NO: 500; and a light chain variable region (VL) that comprises (d) a LCDR1 of SEQ ID NO: 600, (e) a LCDR2 of SEQ ID NO: 700, and (f) a LCDR3 of SEQ ID NO: 800.

In some embodiments, the antibody or antigen-binding fragment thereof specifically binds to an epitope comprising, consisting essentially of, or consisting of amino acid residues 29-139 of human 4Ig-B7H3 (SEQ ID NO: 801). In some embodiments, the antibody or antigen-binding fragment thereof specifically binds to an epitope comprising, consisting essentially of, or consisting of amino acid residues 243-357 of human 4Ig-B7H3 (SEQ ID NO: 801).

In some embodiments, the antibody or antigen-binding fragment thereof specifically binds to IgV1 domain of human 4Ig-B7H3. In some embodiments, the antibody or antigen-binding fragment thereof specifically binds to IgV2 domain of human 4Ig-B7H1³. In some embodiments, the antibody or antigen-binding fragment thereof specifically binds to human 4Ig-B7H3, and binds to both IgV1 and IgV2 domains of human 4Ig-B7H3.

In some embodiments, the antibody or antigen-binding fragment thereof specifically binds to human 4Ig-B7-13, and does not bind to an epitope comprising, consisting essentially of, or consisting of amino acid residues 145-238 of human 4Ig-B7H3 (SEQ ID NO: 801). In some embodiments, the antibody or antigen-binding fragment thereof specifically binds to human 4Ig-B7H3, and does not bind to an epitope comprising, consisting essentially of, or consisting of amino acid residues 363-456 of human 4Ig-B7H3 (SEQ ID NO: 801). In some embodiments, the antibody or antigen-binding fragment thereof specifically binds to human 4Ig-B7H3, and does not bind to IgC1 domain of human 4Ig-B7H3. In some embodiments, the antibody or antigen-binding fragment thereof specifically binds to human 4Ig-B7H3, and does not bind to IgC2 domain of human 4Ig-B7H3. In some embodiments, the antibody or antigen-binding fragment thereof does not bind to either IgC1 nor IgC2 domain of human 4Ig-B7H3. In some embodiments, the antibody or antigen-binding fragment thereof specifically binds to an epitope that does not overlap with the epitope of reference antibody DS-7300.

### 6. BRIEF DESCRIPTION OF FIGURES

FIG. 1 depicts the ADC direct killing on NCI-H1650 cell line.

FIG. 2 depicts the ADC direct killing on Capan-1 cell line.

FIG. 3 depicts the ADC direct killing on MDA-MB-453 (B7H3 negative) cell line.

FIG. 4 depicts the ADC direct killing on NCI-H1650 cell line.

FIG. 5 depicts the ADC direct killing on Capan-1 cell line.

FIG. 6 depicts the ADC direct killing on MDA-MB-453 (B7H3 negative) cell line.

FIG. 7 depicts the ADC direct killing on NCI-H1650 cell line.

FIG. 8 depicts the ADC direct killing on Capan-1 cell line.

FIG. 9 depicts the ADC direct killing on MDA-MB-453 (B7H3 negative) cell line.

FIG. 10 depicts the ADC direct killing on NCI-H1650 cell line.

FIG. 11 depicts the ADC direct killing on NCI-1-1048 cell line.

FIG. 12 depicts the ADC direct killing on MDA-MB-453 (B7H3 negative) cell line.

FIG. 13 depicts the ADC direct killing on NCI-H1650 cell line.

FIG. 14 depicts the ADC direct killing on NCI-H1048 cell line.

FIG. 15 depicts the ADC direct killing on MDA-MB-453 (B7H3 negative) cell line.

FIG. 16 depicts the ADC bystander killing on NCI-H358/MDA-MB-453 (nano-Luc) in a co-culture assay.

FIG. 17 depicts the ADC direct killing on MDA-M/B-453 (nano-Luc) in a cell culture assay. The tested ADCs do not show stronger non-specific killing than isotype ADC (isotype antibody-GGFG-DXd-DAR8 conjugate) on this system.

FIG. 18 depicts the ADC bystander killing on NCI-H358/MDA-MB-453 (nano-Luc) in a co-culture assay.

FIG. 19 depicts the ADC direct killing on MDA-MB-453 (nano-Luc) in a cell culture assay. The tested ADCs do not show stronger non-specific killing than isotype ADC (isotype antibody-GGFG-DXd-DAR8 conjugate) on this system.

FIG. 20 depicts the ADC bystander killing on NCI-H358/MDA-MB-453 (nano-Luc) in a co-culture assay.

FIG. 21 depicts the ADC direct killing on MDA-MB-453 (nano-Luc) in a co-culture assay. The tested ADCs do not show stronger non-specific killing than isotype ADC (isotype antibody-GGFG-DXd-DAR8 conjugate) on this system.

FIG. 22 depicts the ADC bystander killing on NCI-H358/MDA-MB-453 (nano-Luc) in a co-culture assay.

FIG. 23 depicts the ADC direct killing on MDA-MB-453 (nano-Luc) in a co-culture assay. The tested ADCs do not show stronger non-specific killing than isotype ADC (isotype antibody-GGFG-DXd-DAR8 conjugate) on this system.

FIG. 24 depicts the ADC3-1 mouse plasma stability.

FIG. 25 depicts the ADC3-1 human plasma stability.

FIG. 26 depicts the ADC3-2 mouse plasma stability.

FIG. 27 depicts the ADC3-2 human plasma stability.

FIG. 28 depicts the ADC3-4 mouse plasma stability.

FIG. 29 depicts the ADC3-4 human plasma stability.

FIG. 30 depicts the HIC-HPLC profile of ADC3-A (HIC method 2).

FIG. 31A depicts the averaged results of an ADC Efficacy Study of ADC3-1, ADC3-3, ADC3-14, and ADC3-15.

FIG. 31B depicts the results from the vehicle group in the ADC Efficacy Study of FIG. 31A.

FIG. 31C depicts the results from the ADC3-A (3 mpk) group in the ADC Efficacy Study of FIG. 31A.

FIG. 31D depicts the results from the ADC3-A (10 mpk) group in the ADC Efficacy Study of FIG. 31A.

FIG. 31E depicts the results from the ADC3-1 (3 mpk) group in the ADC Efficacy Study of FIG. 31A.

FIG. 31F depicts the results from the ADC3-1 (10 mpk) group in the ADC Efficacy Study of FIG. 31A.

FIG. 31G depicts the results from the ADC3-3 (3 mpk) group in the ADC Efficacy Study of FIG. 31A.

FIG. 31H depicts the results from the ADC3-3 (10 mpk) group in the ADC Efficacy Study of FIG. 31A.

FIG. 31I depicts the results from the ADC3-14 (3 mpk) group in the ADC Efficacy Study of FIG. 31A.

FIG. 31J depicts the results from the ADC3-14 (10 mpk) group in the ADC Efficacy Study of FIG. 31A.

FIG. 31K depicts the results from the ADC3-15 (3 mpk) group in the ADC Efficacy Study of FIG. 31A.

FIG. 31L depicts the results from the ADC3-15 (10 mpk) group in the ADC Efficacy Study of FIG. 31A.

FIG. 32A depicts the averaged results of an ADC Efficacy Study of ADC3-2, ADC3-3, ADC3-4, ADC3-5, and ADC3-6.

FIG. 32B depicts the results from the vehicle group in the ADC Efficacy Study of FIG. 32A.

FIG. 32C depicts the results from the ADC3-A (10 mpk) group in the ADC Efficacy Study of FIG. 32A.

FIG. 32D depicts the results from the ADC3-2 (3 mpk) group in the ADC Efficacy Study of FIG. 32A.

FIG. 32E depicts the results from the ADC3-4 (3 mpk) group in the ADC Efficacy Study of FIG. 32A.

FIG. 32F depicts the results from the ADC3-5 (3 mpk) group in the ADC Efficacy Study of FIG. 32A.

FIG. 32G depicts the results from the ADC3-6 (3 mpk) group in the ADC Efficacy Study of FIG. 32A.

FIG. 33A depicts the averaged results of an ADC Efficacy Study of ADC3-8, and ADC3-9.

FIG. 33B depicts the results from the vehicle group in the ADC Efficacy Study of FIG. 33A.

FIG. 33C depicts the results from the ADC3-A (10 mpk) group in the ADC Efficacy Study of FIG. 33A.

FIG. 33D depicts the results from the ADC3-8 (3 mpk) group in the ADC Efficacy Study of FIG. 33A.

FIG. 33E depicts the results from the ADC3-9 (3 mpk) group in the ADC Efficacy Study of FIG. 33A.

FIG. 34A depicts the averaged results of an ADC Efficacy Study of ADC3-10, ADC3-11, ADC3-12, and ADC3-13.

FIG. 34B depicts the results from the vehicle group in the ADC Efficacy Study of FIG. 34A.

FIG. 34C depicts the results from the ADC3-A (10 mpk) group in the ADC Efficacy Study of FIG. 34A.

FIG. 34D depicts the results from the ADC3-10 (3 mpk) group in the ADC Efficacy Study of FIG. 34A.

FIG. 34E depicts the results from the ADC3-11 (3 mpk) group in the ADC Efficacy Study of FIG. 34A.

FIG. 34F depicts the results from the ADC3-12 (3 mpk) group in the ADC Efficacy Study of FIG. 34A.

FIG. 34G depicts the results from the ADC3-13 (3 mpk) group in the ADC Efficacy Study of FIG. 34A.

FIG. 35A depicts the averaged results of an ADC Efficacy Study of ADC3-1, ADC3-3, ADC3-14, and ADC3-15.

FIG. 35B depicts the results from the vehicle group in the ADC Efficacy Study of FIG. 35A.

FIG. 35C depicts the results from the ADC3-A (3 mpk) group in the ADC Efficacy Study of FIG. 35A.

FIG. 35D depicts the results from the ADC3-A (10 mpk) group in the ADC Efficacy Study of FIG. 35A.

FIG. 35E depicts the results from the ADC3-1 (3 mpk) group in the ADC Efficacy Study of FIG. 35A.

FIG. 35F depicts the results from the ADC3-1 (10 mpk) group in the ADC Efficacy Study of FIG. 35A.

FIG. 35G depicts the results from the ADC3-3 (3 mpk) group in the ADC Efficacy Study of FIG. 35A.

FIG. 35H depicts the results from the ADC3-3 (10 mpk) group in the ADC Efficacy Study of FIG. 35A.

FIG. 35I depicts the results from the ADC3-14 (3 mpk) group in the ADC Efficacy Study of FIG. 35A.

FIG. 35J depicts the results from the ADC3-14 (10 mpk) group in the ADC Efficacy Study of FIG. 35A.

FIG. 35K depicts the results from the ADC3-15 (3 mpk) group in the ADC Efficacy Study of FIG. 35A.

FIG. 35L depicts the results from the ADC3-15 (10 mpk) group in the ADC Efficacy Study of FIG. 35A.

### DETAILED DESCRIPTION

Provided herein are compounds, compositions, ADCs, and methods useful for treating a wide variety of human cancers.

Within the present disclosure, it is understood that the disclosure is not limited to the particular methods and/or experimental conditions described, as such methods and conditions may vary. It is also to be understood that the terminology used herein is for the purpose of describing particular embodiments only, and is not intended to be limiting.

Although any methods and materials similar or equivalent to those described herein can be used in the practice or testing of the present disclosure, the preferred methods and materials are now described. All patents, applications, and non-patent publications mentioned in this specification are incorporated herein by reference in their entireties.

### 7.1. Definitions

When referring to the compounds provided herein, the following terms have the following meanings unless indicated otherwise. Unless defined otherwise, all technical and scientific terms used herein have the same meaning as is commonly understood by one of ordinary skill in the art to which this disclosure pertains. In the event that there is a plurality of definitions for a term provided herein, these Definitions prevail unless stated otherwise.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with amounts or weight percentage of ingredients of a composition, mean an amount or weight percent that is recognized by one of ordinary skill in the art to provide a pharmacological effect equivalent to that obtained from the specified amount or weight percent. In certain embodiments, the terms “about” and “approximately,” when used in this context, contemplate an amount or weight percent within 30%, within 20%, within 15%, within 10%, or within 5%, of the specified amount or weight percent.

As used herein, and unless otherwise specified, the terms “about” and “approximately,” when used in connection with a numeric value or range of values that is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 2θ (or ±0.2 degree 2θ) while still describing the particular XRPD peak.

The term “or” is used to mean, and is used interchangeably with, the term “and/or” unless the context clearly indicates otherwise.

The terms “administration” and “administering,” as used herein, when applied to an animal, human, subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell.

The term “subject” or “patient” herein includes any organism, preferably an animal, more preferably a mammal (e.g., rat, mouse, dog, cat, rabbit, primate) and most preferably a human (e.g., a patient having, or at risk of having, a disorder described herein).

“Treating” any disease or disorder refers in one aspect to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically (e.g., stabilization of a discernible symptom), physiologically (e.g., stabilization of a physical parameter), or both.

The term “effective amount” in connection with a compound means an amount capable of alleviating, in whole or in part, symptoms, or slowing or halting further progression or worsening of those symptoms. As will be apparent to those skilled in the art, it is to be expected that the effective amount of a compound disclosed herein may vary depending on the severity of the indication being treated.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VTH) and a heavy chain constant region. The heavy chain constant region is comprised of three domains, CH1, CH2, and CH3. Each light chain is comprised of a light chain variable region (abbreviated herein as VL or Vκ) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The positions of the CDRs and framework regions can be determined using various well-known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997); Lefranc, M.-P., The Immunologist, 7, 132-136 (1999); Lefranc, M.-P. et al., Dev. Comp. Immunol., 27, 55-77 (2003)).

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA, and IgY), or subclass (e.g., IgG1, IgG2, IgG3, IgG4, IgA1, and IgA2).

The term “monoclonal antibody” or “mAb” or “Mab” herein means a population of substantially homogeneous antibodies, i.e., the antibody molecules in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. Monoclonal antibodies can be obtained by methods known to those skilled in the art. See, for example, Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: a LABORATORY MANUAL, Cold Spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

Unless otherwise indicated, an “antigen-binding fragment” means antigen-binding fragments of antibodies, i.e., antibody fragments that retain the ability to bind specifically to the antigen bound by the full-length antibody, e.g., fragments that retain one or more CDR regions. Examples of antigen-binding fragments include, but are not limited to, Fab, Fab′, F(ab′)2, and Fv fragments; diabodies; linear antibodies; single-chain antibody molecules, e.g., single chain Fv (ScFv); nanobodies and antibodies formed from antibody fragments; and bicyclic peptides (Hurov, K. et al, 2021. Journal for ImmunoTherapy of Cancer, 9(11)).

As used herein, an antibody or antigen-binding antibody fragment “specifically binds” or “selectively binds” to an antigen (e.g., a protein), meaning the antibody exhibits preferential binding to that target as compared to other proteins, but this specificity does not require absolute binding specificity. A “specific” or “selective” binding reaction is determinative of the presence of the antigen in a heterogeneous population of proteins and other biologics, for example, in a blood, serum, plasma, or tissue sample. Thus, under certain designated immunoassay conditions, the antibodies or antigen-binding fragments thereof specifically bind to a particular antigen at least two times greater when compared to the background level and do not specifically bind in a significant amount to other antigens present in the sample. In one aspect, under designated immunoassay conditions, the antibody or antigen-binding fragment thereof specifically binds to a particular antigen at least ten times greater when compared to the background level of binding and does not specifically bind in a significant amount to other antigens present in the sample.

The term “human antibody” herein means an antibody that comprises only human immunoglobulin protein sequences. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, a humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification, or for other reasons.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H}I interfaces, or V_{H}/V_{L} interfaces of antibodies (see, e.g., US 2011/0287009, US2007/0178552, WO 96/027011, WO 98/050431, and Zhu et al., 1997, Protein Science 6:781-788). In some embodiments, knob-into-holes ensure the correct pairing of two different heavy chains together during the manufacture of antibodies. For example, antibodies having knob-into-hole amino acids in their Fc regions can further comprise single variable domains linked to each Fe region, or further comprise different heavy chain variable domains that pair with similar or different light chain variable domains. Knob-into-hole technology can also be used in the VI or VL regions to also ensure correct pairing.

Examples of algorithms that are suitable for determining percent sequence identity and sequence similarity are the BLAST algorithms, which are described in Altschul et al., Nuc. Acids Res. 25:3389-3402, 1977; and Altschul et al, J. Mol. Biol. 215:403-410, 1990. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89:10915), alignments (B) of 50, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, (1988), which has been incorporated into the ALIGN program (version 2.0), using a PAM120 weight residue table, a gap length penalty of 12, and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

In some aspects, the present disclosure provides compositions, e.g., pharmaceutically acceptable compositions, which include anti-B7H3 antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal, or epidermal administration (e.g., by injection or infusion).

The term “human 4Ig-B7H3” refers to the 4Ig isoform of a type I transmembrane protein B7H3 in humans. Human 4Ig-B7H3 (Uniprot Q5ZPR3) has the following sequence:

>sp|Q5ZPR3|CD276_HUMAN CD276 antigen OS = Homo sapiens OX = 9606 GN-CD276
PE = 1 SV = 1
(SEQ ID NO: 802)
MLRRRGSPGM GVHVGAALGA LWFCLTGALE VQVPEDPVVA LVGTDATLCC SFSPEPGESL
AQLNLIWQLT DTKQLVHSFA EGQDQGSAYA NRTALFPDLL AQGNASLRLQ RVRVADEGSF
TCFVSIRDFG SAAVSLQVAA PYSKPSMILE PNKDLRPGDT VTITCSSYQG YPEAEVEWQD
GQGVPLIGNV TTSQMANEQG LFDVHSILRV VLGANGTYSC LVRNPVLQQD AHSSVTITPQ
RSPTGAVEVQ VPEDPVVALV GTDATERCSF SPEPGFSLAQ LNLIWQLTDT KQLVHSFTEG
RDQGSAYANR TALFPDLLAQ GNASLRLQRV RVADEGSFTC FVSIRDFGSA AVSLQVAAPY
SKPSMTLEPN KDLRPGDTVT ITCSSYRGYP EAEVFWQDGQ GVPLIGNVTT SQMANEQGLF
DVHSVLRVVL GANGTYSCLV RNPVLQQDAH GSVTITGQPM TEPPEALWVT VGLSVCLIAL
LVALAFVCWR KIKQSCEEEN AGAEDQDGEG EGSKTALQPL KHSDSKEDDG QEIA

The term “toxin” or “payload” or “cytotoxic agent” is used herein to reference a molecule that inhibits or reduces the expression of molecules in cells, inhibits or reduces the function of cells, induces apoptosis of cells, and/or causes death of cells. The term includes radioactive isotopes, chemotherapeutic agents, and toxins such as small molecule toxins or enzymatically active toxins of bacterial, fungal, plant, or animal origin, including fragments and/or variants thereof. Examples of cytotoxic agents include, but are not limited to, auristatins (e.g., auristatin E, auristatin F, MMAE, and MMAF), auromycins, maytansinoids, pyrrolobenzodiazepine (PBD), ricin, ricin A-chain, combrestatin, duocarmycins, dolastatins, doxorubicin, daunorubicin, taxols, cisplatin, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, and calicheamicin, as well as radioisotopes such as At211, I131, I125, Y90, Re186, Re188, Sm153, Bi212 or 213, P32, and Lu177.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (triose) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, and not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of a molecule via its amino group to form an amide linkage with the rest of the molecule (i.e., a glucamide).

As used herein, “binding agent” refers to any molecule, e.g., antibody, capable of binding with specificity to a given binding partner, e.g., antigen.

As used herein, the term “amino acid” refers to an organic compound that contains amine (—NH₂) and carboxyl (COOH) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, ornithine, and citrulline.

As used herein “peptide,” in its various grammatical forms, is defined in its broadest sense to refer to a compound of two or more subunit amino acids, amino acid analogs, or other peptidomimetics. The subunits may be linked by peptide bonds or by other bonds, for example, ester, ether, and the like. As used herein, the term “amino acid” refers to either natural and/or unnatural, proteinogenic or non-proteinogenic, or synthetic amino acids, including glycine and both the D or L optical isomers, and amino acid analogs and peptidomimetics. If the peptide chain is short, e.g., two, three, or more amino acids, it is commonly called an oligopeptide. If the peptide chain is longer, the peptide is typically called a polypeptide or a protein. Full-length proteins, analogs, mutants, and fragments thereof are encompassed by the definition. The terms also include post-expression modifications of the polypeptide, for example, glycosylation, acetylation, phosphorylation and the like. Furthermore, as ionizable amino and carboxyl groups are present in the molecule, a particular peptide may be obtained as an acidic or basic salt, or in neutral form. A peptide may be obtained directly from the source organism, or may be recombinantly or synthetically produced.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In some embodiments, the cancer is colorectal carcinoma, prostate cancer, breast cancer, lung cancer (e.g., non-small cell lung cancer, such as squamous non-small cell lung cancer, or small cell lung cancer), or esophageal carcinoma (e.g., esophageal squamous cell carcinoma.

As used herein, the term “cell-killing activity” refers to the activity that decreases or reduces the cell viability of the tested cell line.

As used herein, the term “bystander killing” refers to the situation in which the drug from an ADC is released either from the target cell following internalization and degradation of the ADC, or release of the drug within the extracellular space. In both cases, the drug is then taken up by and kills surrounding or bystander cells, which themselves may or may not express the ADC target antigen.

An “alkyl” group is a saturated straight chain or branched non-cyclic hydrocarbon having from 1 to 10 carbon atoms, typically from 1 to 8 carbons or, in some embodiments, from 1 to 6, 1 to 4, or 2 to 6 carbon atoms. Representative alkyl groups include -methyl, -ethyl, -n-propyl, -n-butyl, -n-pentyl and n-hexyl; while saturated branched alkyls include -isopropyl, -sec-butyl, -isobutyl, -tert-butyl, -isopentyl, 2-methylpentyl, 3methylpentyl, 4-methylpentyl, 2,3-dimethylbutyl and the like. An alkyl group can be substituted or unsubstituted. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₅)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, -3-octenyl and the like. The double bond of an alkenyl group can be unconjugated or conjugated to another unsaturated group. An alkenyl group can be unsubstituted or substituted.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₄ alkynyl. Examples of alkynyl moieties include, but are not limited to ethynyl, propynyl, and butynyl.

A “cycloalkyl” group is a saturated or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., CH₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy; and 6 to 10 ring carbon atoms, i.e., C₆₋₁₀ aryloxy. Examples of aryloxy moieties include, but are not limited to phenoxy, naphthoxy, and anthroxy.

As used herein, “haloalkyl” refers to alkyl, as defined above, wherein the alkyl includes at least one substituent selected from a halogen, for example, fluorine (F), chlorine (C), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur, and nitrogen. In certain embodiments, the heteroaryl ring system is monocyclic or bicyclic. Non-limiting examples include, but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, II-benzo[d]imidazolyl), imidazopyridyl (for example, azabenzimidazolyl, 3Himidazo[4,5-b]pyridyl, or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is a non-aromatic cycloalkyl in which one to four of the ring carbon atoms are independently replaced with a heteroatom independently selected from the group consisting of O, S, and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated, and saturated ring systems, such as, for example, imidazolyl, imidazolinyl and imidazolidinyl groups. The term “heterocyclyl” includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3—, 4-, 5-, or 6-substituted, or disubstituted with various substituents such as those listed below.

A “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

A “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include but are not limited to 4-ethyl-morpholinyl, 4-propylmorpholinyl, furan-2-yl methyl, furan-3-yl methyl, pyridine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

An “alkoxy” or “alkoxyl” group is —O(alkyl), wherein alkyl is defined above.

An “alkoxyalkyl” group is -(alkyl)O(alkyl), wherein each alkyl is independently as defined above.

An “amine” group is a radical of the formula: —NH₂.

A “hydroxyl amine” group is a radical of the formula: N(R^{#})OH or NHOH, wherein R⁴ is a substituted or unsubstituted alkyl, cycloalkyl, cycloalkylalkyl, aryl, aralkyl, heterocyclyl, or heterocyclylalkyl group as defined herein.

An “alkoxyamine” group is a radical of the formula: —N(R^{#})O-alkyl or —NHO-alkyl, wherein R^{#} is as defined above.

An “aralkoxyamine” group is a radical of the formula: N(R^{#})O-aryl or NHOaryl, wherein R is as defined above.

An “alkylamine” group is a radical of the formula: NIHalkyl or N(alkyl)₂, wherein each alkyl is independently as defined above.

An “aminocarbonyl” group is a radical of the formula: —C(═O)N(R^{#})₂, —C(═O)NH(R^{#}), or C(═O)NH₂, wherein each Rh is as defined above.

An “acylamino” group is a radical of the formula: NHC(═O)(R^{#}) or N(alkyl)C(═O)(R^{#}), wherein each alkyl and R⁴ are independently as defined above.

An “O(alkyl)aminocarbonyl” group is a radical of the formula: —O(alkyl)C(═O)N(R^{#})₂, —O(alkyl)C(═O)NH(R^{#}), or —O(alkyl)C(═O)NH₂, wherein each R^{#} is independently as defined above.

An “N-oxide” group is a radical of the formula: —N⁺—O⁻.

A “carboxy” group is a radical of the formula: C(═O)OH.

A “ketone” group is a radical of the formula: C(═O)(R^{#}), wherein R^{#} is as defined above.

An “aldehyde” group is a radical of the formula: —CH═O).

An “ester” group is a radical of the formula: C(═O)O(R^{#}) or OC(═O)(R^{#}), wherein R^{#} is as defined above.

A “urea” group is a radical of the formula: —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂^{#}, wherein each alkyl and R are independently as defined above.

An “imine” group is a radical of the formula: —N═C(R^{#})₂ or —C(R^{#})═N(R^{#}) wherein each R^{#} is independently as defined above.

An “imide” group is a radical of the formula: —C(═O)N(R^{#})C(═O)(R^{#}) or N((C═O)(R^{#}))₂, wherein each R^{#} is independently as defined above.

A “urethane” group is a radical of the formula: —OC(═O)N(R^{#})₂, —OC(═)NHI(R^{#}), —N(R^{#})C(═O)O(R^{#}) or —NHC(═O)O(R^{#}), wherein each R^{#} is independently as defined above.

An “amidine” group is a radical of the formula: —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula: —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R is independently as defined above.

An “enamine” group is a radical of the formula: —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), C(R^{#})═C(R^{#})(NH(R^{#})) or —C(R^{#})═C(R^{#})(NH₂), wherein each R^{#} is independently as defined above.

An “oxime” group is a radical of the formula: —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula: —C(═O)N(R^{#})N(R^{#})₂, —C(═O)NHN(R^{#})₂, —C(═O)N(R^{#})NH(R^{#}), —C(═O)N(R^{#})NH, —C(═O)NHNH(R^{#})₂, or —C(═O)NHNH₂, wherein each R^{#} is independently as defined above.

A “hydrazine” group is a radical of the formula: —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula: —C(═N—N(R^{#})₂)(R^{#}), —C(═NNH(R^{#}))(R^{#})₂, —C(═N—NH₂)(R^{#})₂, —N(R^{#})(N═C(R^{#})₂), or —NH(N═C(R^{#})₂), wherein each R^{#} is independently as defined above.

An “azide” group is a radical of the formula: —N₃.

An “isocyanate” group is a radical of the formula: N═C═O.

An “isothiocyanate” group is a radical of the formula: N═C═S.

A “cyanate” group is a radical of the formula: OCN.

A “thiocyanate” group is a radical of the formula: SCN.

A “thioether” group is a radical of the formula; —S(R^{#}), wherein R^{#} is as defined above.

A “thiocarbonyl” group is a radical of the formula: —C(═S)(R^{#}), wherein R^{#} is as defined above.

A “sulfinyl” group is a radical of the formula: —S(═O)(R^{#}), wherein R^{#} is as defined above.

A “sulfone” group is a radical of the formula: —S(═O)₂(R^{#}), wherein R^{#} is as defined above.

A “sulfonylamino” group is a radical of the formula: —NHSO₂(R^{#}) or —N(alkyl)SO₂(R^{#}), wherein each alkyl and R^{#} are defined above.

A “sulfonamide” group is a radical of the formula: —S(═O)₂N(R^{#})₂, —S(═O)₂NH(R^{#}), or —S(═O)₂NH₂, wherein each R^{#} is independently as defined above.

A “phosphonate” group is a radical of the formula: —P(═O)(O(R^{#}))₂, —P(═O)(OH)₂, —OP(═O)(O(R^{#}))(R^{#}), or —OP(═O)(OH)(R^{#}), wherein each R^{#} is independently as defined above.

A “phosphine” group is a radical of the formula: —P(R^{#})₂, wherein each R^{#} is independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

All pharmaceutically acceptable salts, solvates, and/or hydrates of compounds depicted herein are within the scope of the present disclosure.

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving, Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

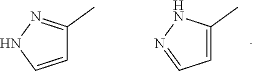

As readily understood by one skilled in the art, a wide variety of functional groups and other structures may exhibit tautomerism and all tautomers of the compounds are within the scope of the present disclosure.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I) sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

Certain groups, moieties, substituents, and atoms are depicted with a wiggly line that intersects a bond or bonds to indicate the atom through which the groups, moieties, substituents, or atoms are bonded. For example, a phenyl group that is substituted with a propyl group depicted as:

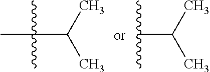

has the following structure:

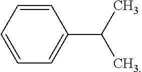

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains.

Illustrations showing substituents bonded to a non-cyclic group through a bond between two atoms are meant to indicate, unless specified otherwise, that the substituent may be bonded to either atom of the bond through which the substituent bond passes, according to techniques set forth herein or which are known in the field to which the instant disclosure pertains. Thus, for example,

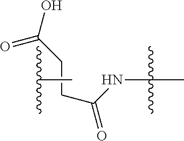

encompasses

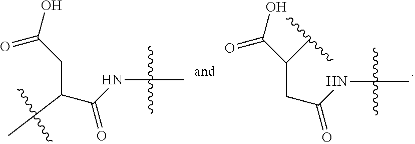 [SMILES: *NC(=O)C(*)CC(=O)O ; *NC(=O)CC(*)C(=O)O]

It should be noted that if there is a discrepancy between a depicted structure and a name for that structure, the depicted structure is to be accorded more weight.

In the claims which follow and in the preceding description, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the disclosure.

### 7.2. Conjugates

Provided herein are anti-B7H3 antibody drug conjugates. In some embodiments, the conjugates comprise an antibody or antigen binding fragment thereof capable of specific binding to human B7H3 (Ab); and a cytotoxic agent (D). Optionally, the cytotoxic agent is covalently attached to the antibody or antigen binding fragment thereof by a linker.

International Publication No. WO 2023/125530, the entire contents of which are incorporated herein by reference, discloses antibody drug conjugates, the linker payload portions of which are suitable for use in the context of the present disclosure, and linker payloads which are suitable for use in the context of the present disclosure. In some embodiments, a linker payload is a linker payload disclosed in WO 2023/125530.

In embodiments, an antibody drug conjugate has Formula (XI):

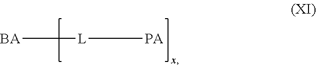

or a pharmaceutically acceptable salt, tautomer, solvate, stereoisomer, enantiomer, or isotopologue thereof,
wherein BA is Ab, as that variable is described with respect to an antibody drug conjugate of the present disclosure (e.g., an antibody drug conjugate of Formula (I)); L is a covalent linker; PA is a payload residue (e.g., a residue of a cytotoxic agent (D), as that variable is described with respect to an antibody drug conjugate of the present disclosure (e.g., an antibody drug conjugate of Formula (I)); and subscript x is from 1 to 30. In some instances, x is n as that variable is described with respect to an antibody drug conjugate of the present disclosure (e.g., an antibody drug conjugate of Formula (I)). In some instances, x is from 1 to 4. In some instances, x is about 1. In some instances, x is about 2. In some instances, x is about 3. In some instances, x is about 4.

In further embodiments, the antibody drug conjugate has Formula (XIa):

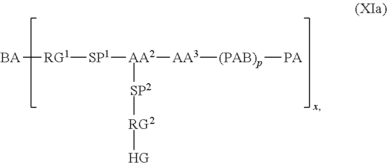

or a pharmaceutically acceptable salt, tautomer, solvate, stereoisomer, enantiomer, isotopologue, or prodrug thereof, wherein RG¹ is a reactive group residue; RG² is an optional reactive group residue; SP and SP² are independently, in each instance, an optional spacer group residue; HG is a hydrophilic residue; PAB is an optional self-immolative unit; subscript p is 0 or 1; and subscript x is from 1 to 30. Values for the remaining variables (e.g., AA², AA³) and alternative values for the variables (e.g., x, p, PAB, HG, RG, RG², SP¹, SP², BA) are as described elsewhere herein.

In some embodiments, x is from 1 to 15. In some embodiments, x is from 2 to 10. In some embodiments, x is from 3 to 9. In one embodiment, x is about 3. In one embodiment, x is about 4. In one embodiment, x is about 5. In one embodiment, x is about 6. In one embodiment, x is about 7. In one embodiment, x is about 8. In one embodiment, x is about 9.

In some embodiments of a compound of Formula (XIa), AA² comprises formula (W):

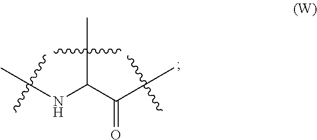 [SMILES: *NC(*)C(*)=O]

and
AA³ is a dipeptide residue of -valine-alanine-, -valine-citrulline-, or

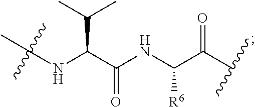

wherein R⁶ is —CH₃, or —(CH₂)₃—NIC(═O)NH₂.

In some embodiments,

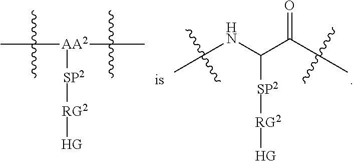

In some embodiments, AA³ is

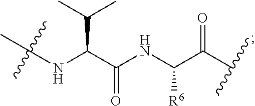

wherein R⁶ is —CH₃, or —(CH₂)₃NHC(═O)NH₂. In further embodiments, R⁶ is —CH₃.

In some embodiments, AA² is an amino acid residue of glycine or

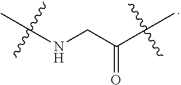 [SMILES: *NCC(*)=O]

In some embodiments, AA² comprises formula (W):

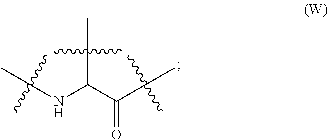 [SMILES: *NC(*)C(*)=O]

and
AA³ is a tetrapeptide residue of -glycine-glycine-phenylalanine-glycine- or

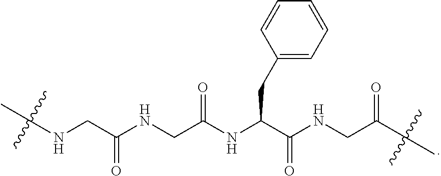 [SMILES: *NCC(=O)NCC(=O)N[C@@H](Cc1ccccc1)C(=O)NCC(*)=O]

In some embodiments, PAB represents —NH—CH2-O—, formula (Y1):

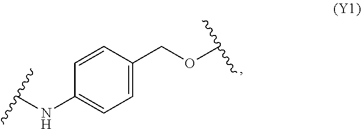 [SMILES: *Nc1ccc(CO*)cc1]

or
formula (Y2):

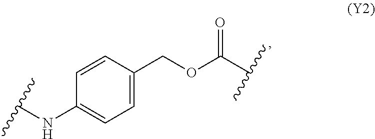 [SMILES: *Nc1ccc(COC(*)=O)cc1]

wherein the

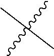

indicates the bond through which the PAB is bonded to the adjacent groups in the formula.

In some embodiments, PAB is —NH—CH₂—O—.

In some embodiments, RG¹ is

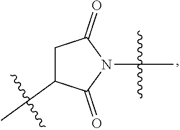 [SMILES: *C1CC(=O)N(*)C1=O]

-(succinimid-3-yl-N)—,

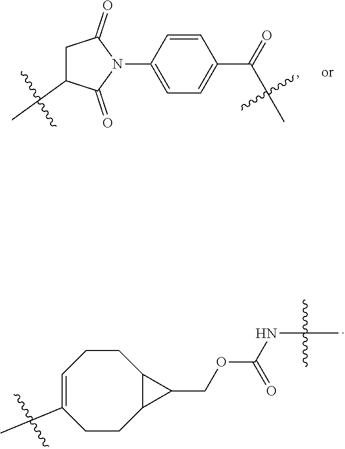 [SMILES: *C(=O)c1ccc(N2C(=O)CC(*)C2=O)cc1 ; *NC(=O)OCC1C2CC/C=C(/*)CCC21]

In some embodiments, RG¹ is

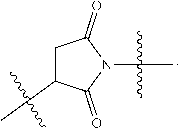 [SMILES: *C1CC(=O)N(*)C1=O]

In some embodiments, RG¹ is

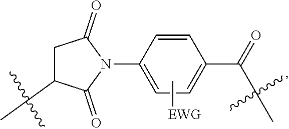

wherein EWG is an electrowithdrawing group, e.g., —CN, —NO₂, halogen, —CF₃, —C(═O)OR¹, or —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

In some embodiments, RG¹ is

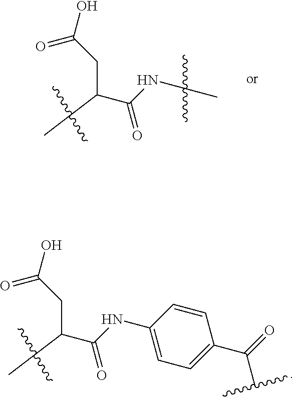 [SMILES: *NC(=O)C(*)CC(=O)O ; *C(=O)c1ccc(NC(=O)C(*)CC(=O)O)cc1]

In some embodiments, RG¹ is

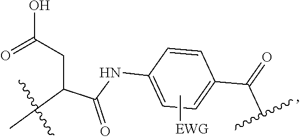

wherein EWG is an electrowithdrawing group, e.g., —CN, —NO₂, halogen, —CF₃, —C(═O)OR¹, or —C(═O)R¹, and R¹ is substituted or unsubstituted alkyl, substituted or unsubstituted cycloalkyl, substituted or unsubstituted aryl, substituted or unsubstituted heterocycloalkyl, or substituted or unsubstituted heteroaryl.

In some embodiments, RG¹ is an opened heterocyclic ring, such as the product resulting from conjugation of the maleimide ring of

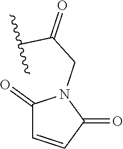 [SMILES: *C(=O)CN1C(=O)C=CC1=O]

with an antibody. It will be appreciated in this regard that conjugation of an antibody to a maleimide ring can occur at either one of the two carbons in the carbon-carbon double bond of the maleimide. Similarly, in the context of an opened ring, conjugation can occur at either one of the two carbon atoms in the double bond. In some embodiments, RG¹ is

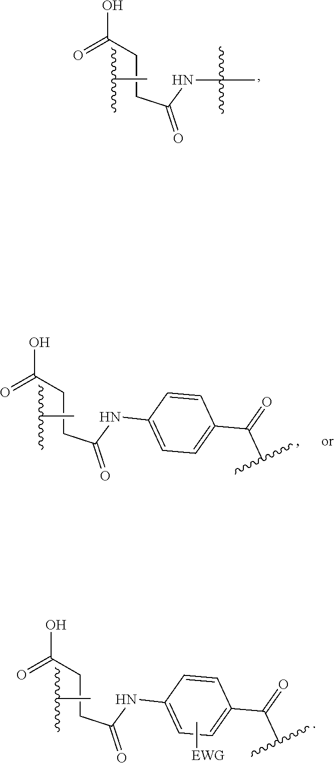

In some embodiments, RG¹ is

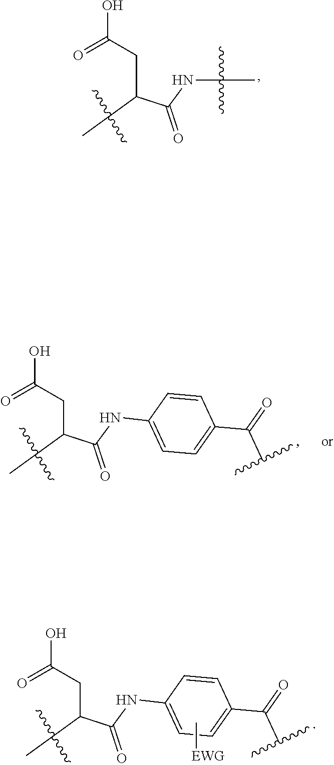

In some embodiments, RG¹ is

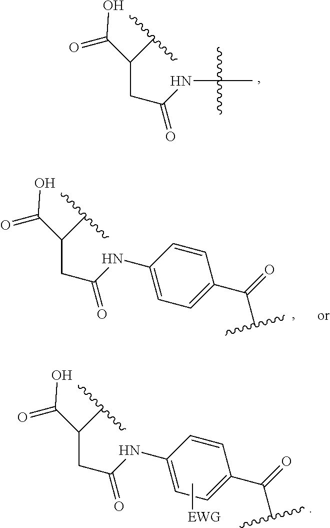

In some embodiments, RG¹ is

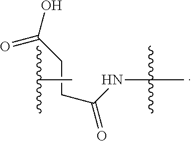

In some embodiments, RG¹ is

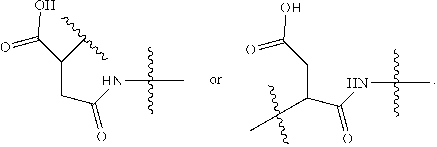 [SMILES: *NC(=O)CC(*)C(=O)O ; *NC(=O)C(*)CC(=O)O]

In some embodiments, RG² is a bond, —C(═O)—NH—, or —NHC(═O)—. In some embodiments, RG² is —C(═O)—NH—.

In some embodiments, SP¹ is —(CH₂)ₙ₁—C(═O)—, —(CH₂CH₂O)ₙ₂—CH₂CH₂—C(═O)—, —CH[—(CH₂)ₙ₃—COOH]—C(═O)—, —CH₂—C(═O)—NH—(CH₂)ₙ₄—C(═O)—, —CH₂—C(═O)—NH—(CH₂)₃—C(═O)—NH—(CH₂)ₙ₄—C(═O)—, or C(═O)—(CH₂)ₙ₅—C(═O)—, wherein each of n1, n2, n3, n4, and n5 independently represents an integer of 1 to 8. In some embodiments, SP¹ is *—CH₂C(O)N(H)CH₂CH₂C(O)—, wherein asterisk marks the bond that connects to RG. In some embodiments, SP¹ is *—(CH₂)₅C(O)—, wherein asterisk marks the bond that connects to RG¹. In some embodiments, SP¹ is *—C(H)(CH₂NH₂)—(CH₂)₂OC(O)N(H)(CH₂)₂C(O)—, wherein asterisk marks the bond that connects to RG¹.

In some embodiments, SP² is —(CH₂)ₙ₆—; and n6 represents an integer of 1 to 8. In some embodiments, n6 is 2.

In some embodiments, HG is

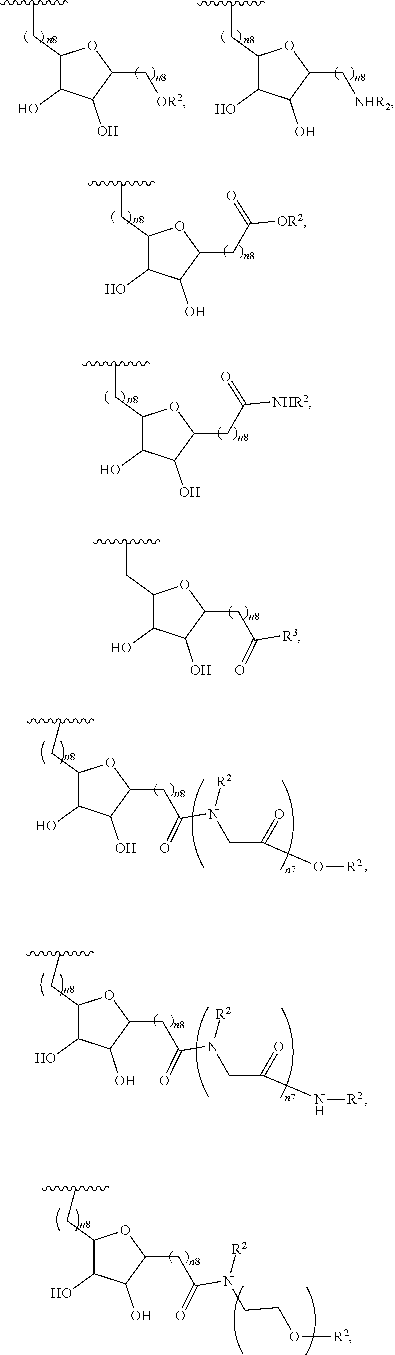

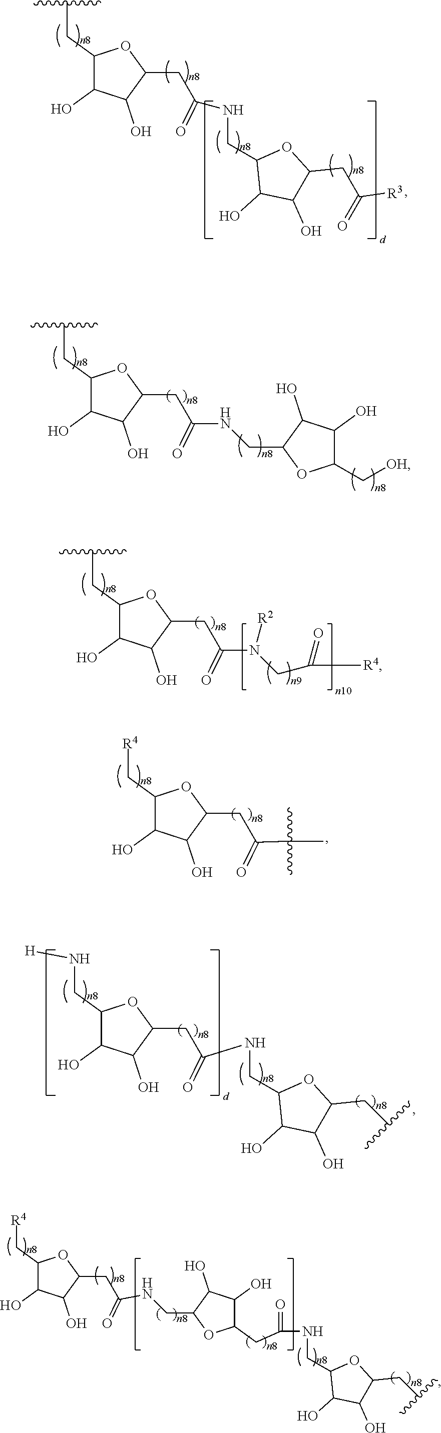

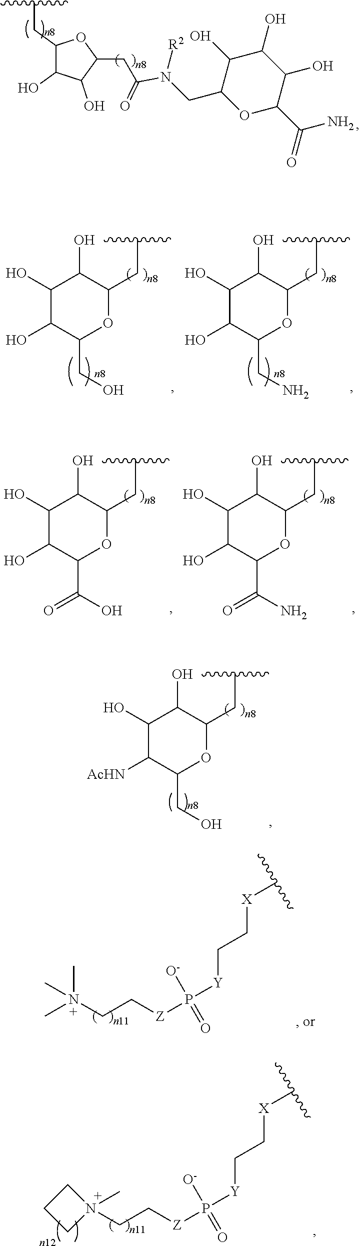

wherein each n7 is independently 1-15; each n8 is independently 0 or 1; each n9 is independently 1 or 2; each n10 is independently an integer of 4 to 16, such as 4, 8, or 12;
each n11 is independently an integer of 0 to 5; n12 is an integer of 0 to 3; d is 0-3; R² is H or Me; R³ is —OH, —NH₂, —NHCH₂—CH₂—(PEG)ₓ-OH, or —NHCH₂—CH₂—(PEG)ₓ-OMe; R⁴ is OH or NH₂; and each of X, Y, and Z is independently —CH₂—, —NH—, —S— or —O—.

In some embodiments, HG is

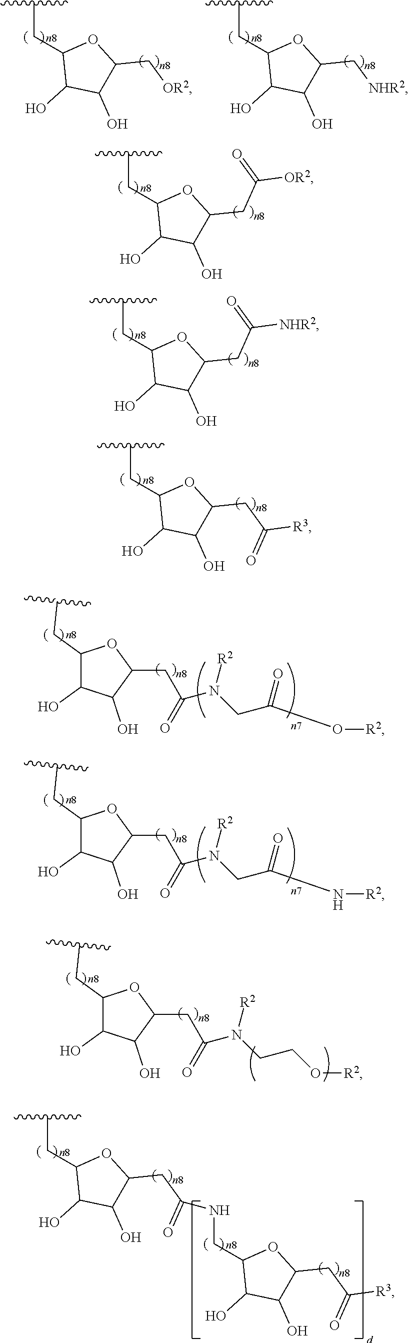

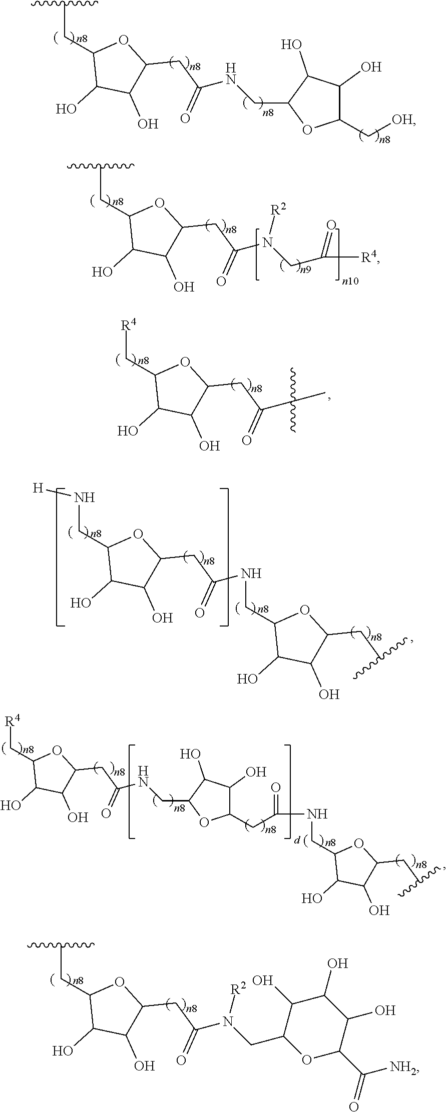

wherein each n7 is independently 1-15; each n8 is independently 0 or 1; each n9 is independently 1 or 2; each n10 is independently an integer of 4 to 16, such as 4, 8, or 12: d is 0-3; R² is H or Me; R³ is —OH, —NH₂, —NHCH₂—CH₂—(PEG)ₓ-OH, or —NHCH₂—CH₂—(PEG)ₓ-OMe; R⁴ is OH or NH₂.

In some embodiments, HG is

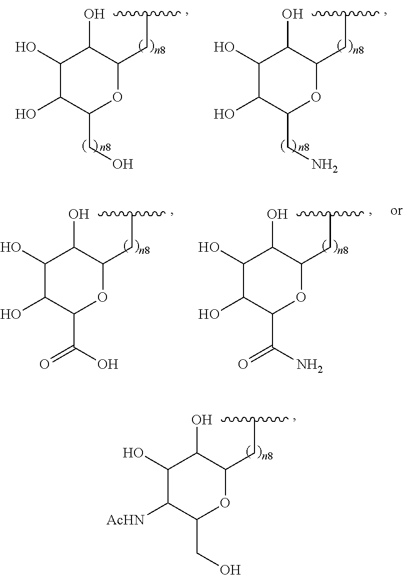

wherein each n8 is independently 0 or 1; and R¹ is H or Me.

In some embodiments, HG is

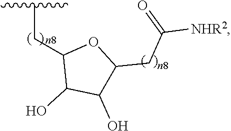

wherein n8 is independently 0 or 1.

In some embodiments, HG is

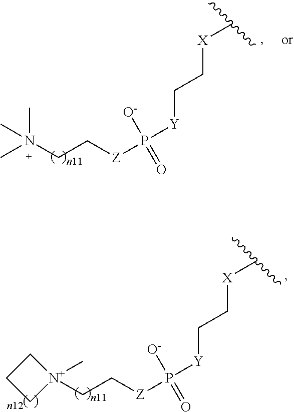

wherein each n11 is independently an integer of 0 to 5; n12 is an integer of 0 to 3; and each of X, Y, and Z is independently —CH₂—, —NH—, —S— or —O—.

In some embodiments, HG is —NHSO₂NH₂, —SO₃H, —SO₂NH₂, —PO₁₃H2, and RG² is a bond.

In some embodiments, each PA independently represents a chromophore functional group.

In some embodiments, each chromophore functional group is independently a functional group selected from a class or subclass of xanthophores, erythrophores, iridophores, leucophores, melanophores, and cyanophores; a class or subclass of fluorophore molecules which are fluorescent chemical compounds that emit light upon exposure to an excitation light; a class or subclass of visual phototransduction molecules; a class or subclass of photophore molecules; a class or subclass of luminescence molecules; and a class or subclass of luciferin compounds.

In some embodiments, each PA is a residue of a cytotoxic agent independently selected from the group consisting of Monomethyl auristatin E (MMAE), Monomethyl auristatin F (MMAF), Monomethyl auristatin D (MMAD), Mertansine (Maytansinoid DMI/DM4), Paclitaxel, Docetaxel, Epothilone B, Epothilone A, CYT997, Auristatin tyramine phosphate, Auristatin aminoquinoline, Halocombstatins, Calicheamicin theta, 7-Ethyl-10-hydroxy-camptothecin (SN-38), Pyrrolobenzodiazepine (PBD), Pancratistatin, Cyclophosphate, Cribrostatin-6, Kitastatin, Turbostatin 1-4, Halocombstatins, Eribulin, Hemiasterlin, PNU, and Silstatins.

In some embodiments each PA independently has formula (D1):

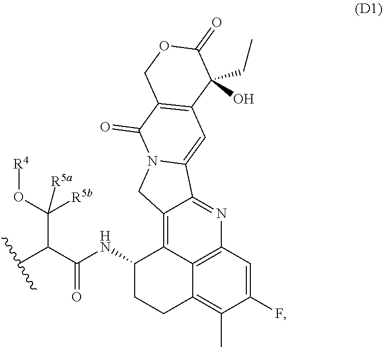

wherein each of R⁴, R^{5a}, and R^{5b} is independently hydrogen, sugar residue, substituted or unsubstituted inorganic or organic acid residue, substituted or unsubstituted C₁₋₈ alkyl, substituted or unsubstituted cycloalkyl, substituted or unsubstituted non-aromatic heterocyclyl, substituted or unsubstituted cycloalkylalkyl, or substituted or unsubstituted heterocyclylalkyl;
R^{5a} and R^{5b} together with the atoms to which they are attached form a substituted or unsubstituted cycloalkyl, or substituted or unsubstituted non-aromatic heterocyclyl.

In some embodiments, R⁴ is hydrogen,

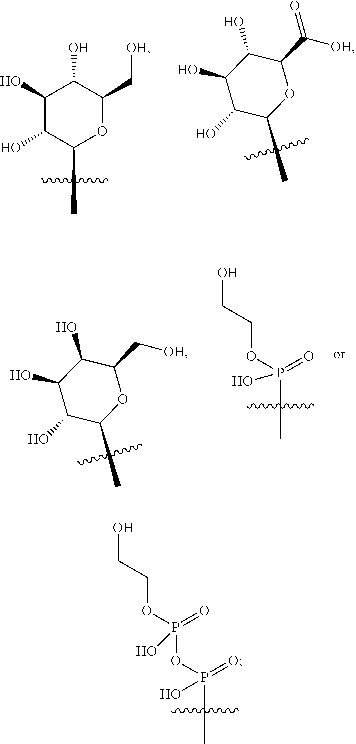

and
wherein each of R^{5a} and R^{5b} is independently H, CH₃, or CF₃; or
R^{5a} and R^{5b} together with the atoms to which they are attached form a substituted or unsubstituted cycloalkyl, substituted or unsubstituted non-aromatic heterocyclyl.

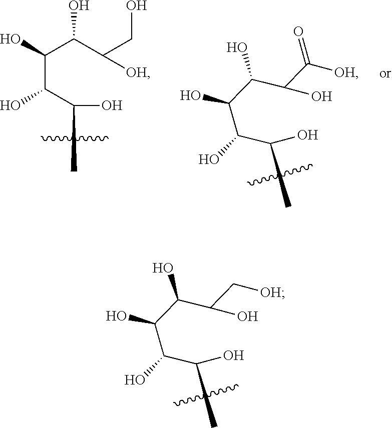

and
wherein each of R^{5a} and R^{5b} is independently H, CH₃, or CF₃; or
R^{5a} and R^{5b} together with the atoms to which they are attached form a substituted or unsubstituted cycloalkyl, substituted or unsubstituted non-aromatic heterocyclyl.

In some embodiments, each PA independently has one of the following structures:

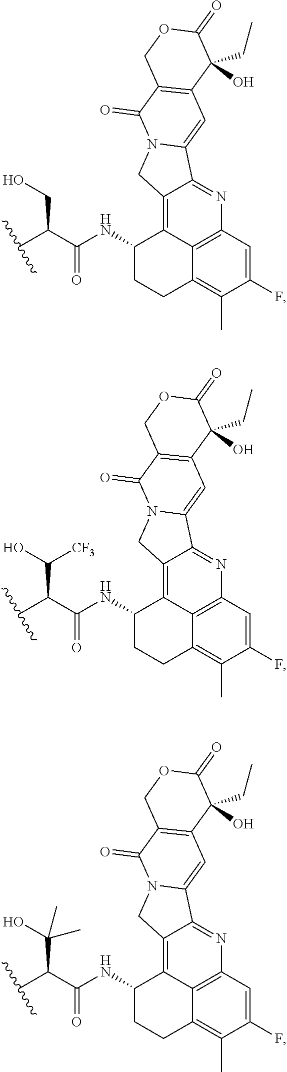

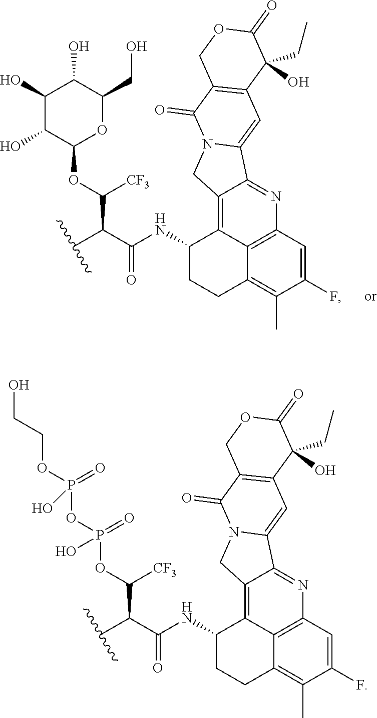 [SMILES: *[C@H](C(=O)N[C@H]1CCc2c(C)c(F)cc3nc4c(c1c23)Cn1c-4cc2c(c1=O)COC(=O)[C@]2(O)CC)C(O[C@@H]1O[C@H](CO)[C@@H](O)[C@H](O)[C@H]1O)C(F)(F)F ; *[C@H](C(=O)N[C@H]1CCc2c(C)c(F)cc3nc4c(c1c23)Cn1c-4cc2c(c1=O)COC(=O)[C@]2(O)CC)C(OP(=O)(O)OP(=O)(O)OCCO)C(F)(F)F]

In some embodiments, each PA independently has formula (D2):

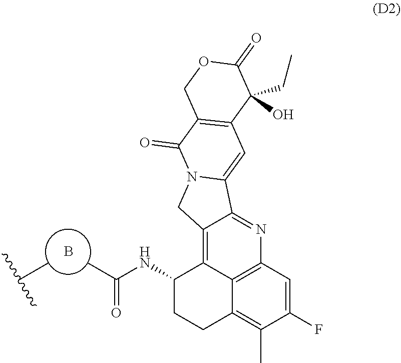

wherein ring B is a substituted or unsubstituted cycloalkyl, substituted or unsubstituted aryl, substituted or unsubstituted heterocyclyl, or substituted or unsubstituted heteroaryl.

In some embodiments, each PA independently has one of the following structures:

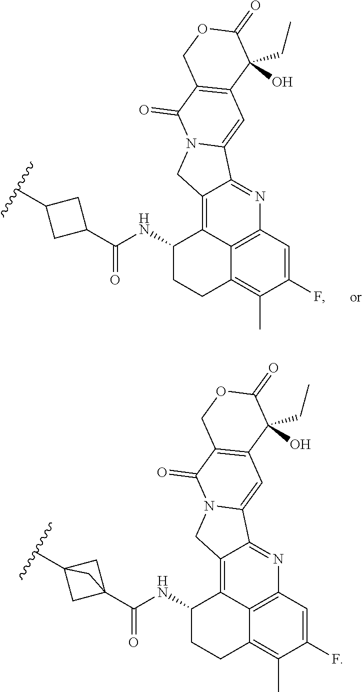

In some embodiments, each PA independently has formula (D3):

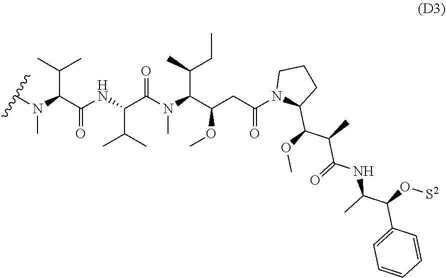

wherein S² is an enzyme hydrolyzable hydrophilic group.

In some embodiments, the S² group is hydrogen or represents one of the following formulas:

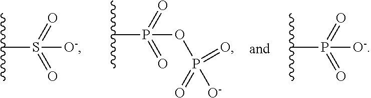

In some embodiments, each PA independently has formula (E1):

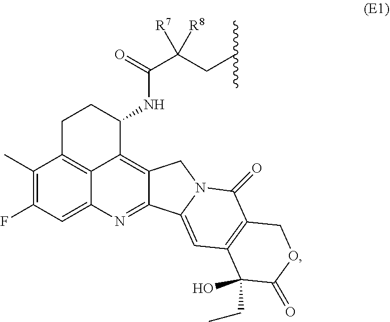

wherein each of R⁷ and R⁸ is, independently, hydrogen, halogen, or alkyl.

In some embodiments, R⁷ and R⁸ are hydrogen.

In some embodiments, R⁷ and R⁸ are methyl.

In some embodiments, R⁷ is methyl and R⁸ is F.

In some embodiments, the carbon to which R⁷ and R⁸ connect is in the S configuration.

In some embodiments, the carbon to which R⁷ and R⁸ connect is in the R configuration.

In some embodiments, each PA independently has the following formula:

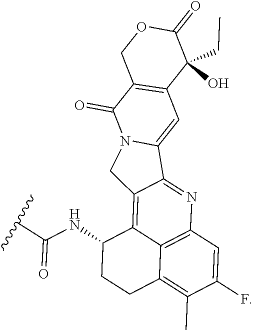 [SMILES: *C(=O)N[C@H]1CCc2c(C)c(F)cc3nc4c(c1c23)Cn1c-4cc2c(c1=O)COC(=O)[C@]2(O)CC]

In some embodiments, each PA is independently Dxd, or independently has the following formula:

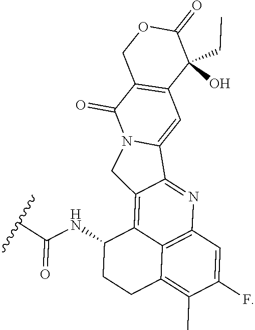 [SMILES: *C(=O)N[C@H]1CCc2c(C)c(F)cc3nc4c(c1c23)Cn1c-4cc2c(c1=O)COC(=O)[C@]2(O)CC]

In some embodiments, each PA independently has the following formula:

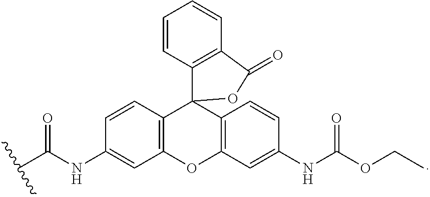 [SMILES: *C(=O)Nc1ccc2c(c1)Oc1cc(NC(=O)OCC)ccc1C21OC(=O)c2ccccc21]

In further embodiments, the antibody drug conjugate has Formula (XIb):

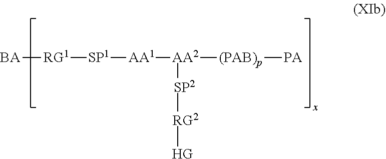

or a pharmaceutically acceptable salt, tautomer, solvate, stereoisomer, enantiomer, isotopologue, or prodrug thereof, wherein AA² comprises formula (W):

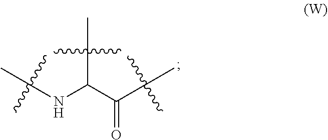 [SMILES: *NC(*)C(*)=O]

and

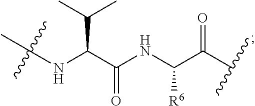

AA¹ is a dipeptide residue of -valine-alanine-, -valine-citrulline-, or
wherein R⁶ is —CH₃, or —(CH₂)₃—NHC(═O)NH₂. Values for the remaining variables (e.g., x, p, BA, HG, RG¹, RG², SP¹, SP², PAB, PA) and alternative values for the variables (e.g., AA¹, AA²) are as described elsewhere herein, for example, with respect to compounds of Formula XIa.

In some embodiments, AA² comprises formula (W):

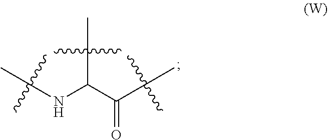 [SMILES: *NC(*)C(*)=O]

and
AA¹ is a tetrapeptide residue of -glycine-glycine-phenylalanine-glycine- or

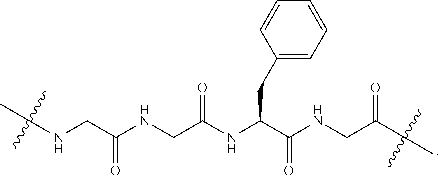 [SMILES: *NCC(=O)NCC(=O)N[C@@H](Cc1ccccc1)C(=O)NCC(*)=O]

In further embodiments, the antibody drug conjugate has Formula (XIc):

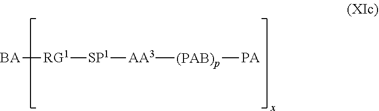

or a pharmaceutically acceptable salt, tautomer, solvate, stereoisomer, enantiomer, isotopologue, or prodrug thereof,
wherein AA³ is a dipeptide residue of -valine-alanine-, -valine-citrulline-, or

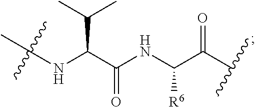

wherein R⁶ is —CH₃, or —(CH₂)₃—NHC(═O)NH₂. Values for the remaining variables (e.g., BA, RG¹, SP¹, PAB, p, PA, x) and alternative values for the variables (AA³, R⁶) are as described elsewhere herein, for example, with respect to a compound of Formula XIa.

In some embodiments (e.g., of a compound of Formula (XIc)), AA³ is a tetrapeptide residue of -glycine-glycine-phenylalanine-glycine- or

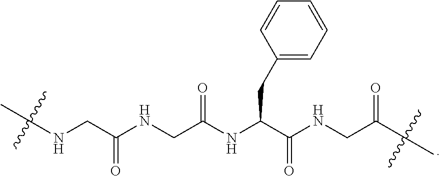 [SMILES: *NCC(=O)NCC(=O)N[C@@H](Cc1ccccc1)C(=O)NCC(*)=O]

In some embodiments, an antibody drug conjugate is selected from one of the following compounds, or a pharmaceutically acceptable salt, tautomer, solvate, stereoisomer, enantiomer, or isotopologue thereof.

| Example No. | Antibody drug conjugate |
|---|---|
| ADC1-2 | 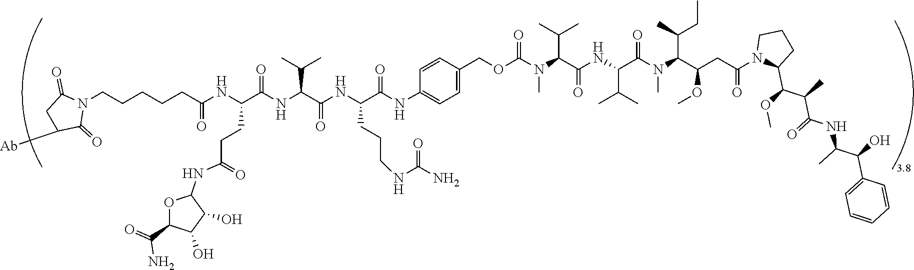 |
| ADC1-4 | 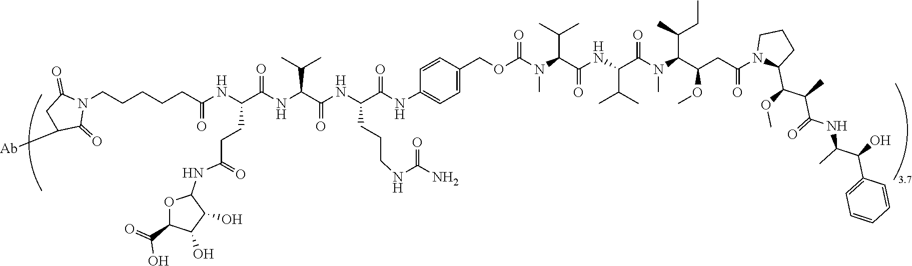 |
| ADC1-11 | 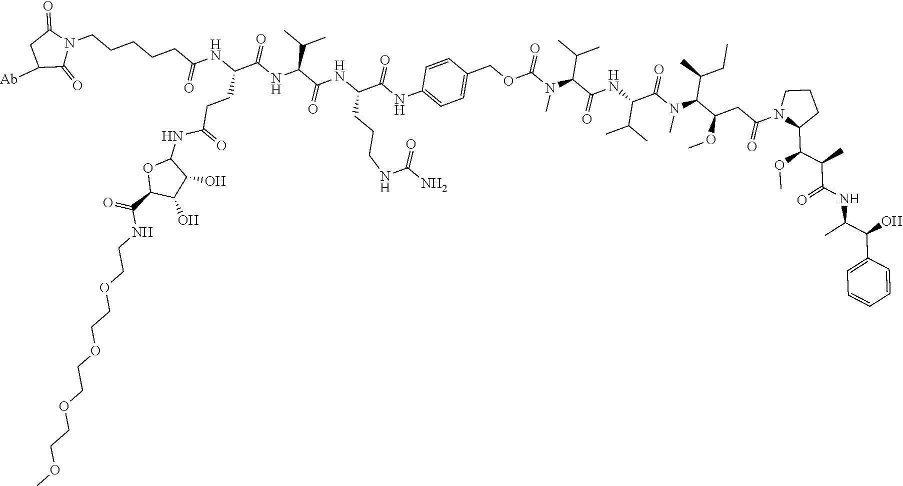 |
| ADC2-2 | 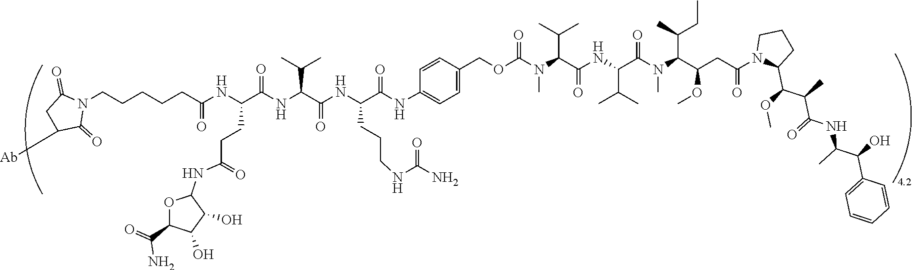 |
| ADC2-4 | 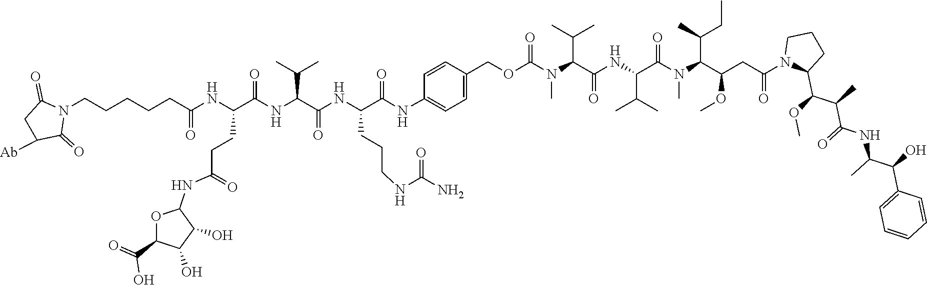 |
| ADC2-6-2 | 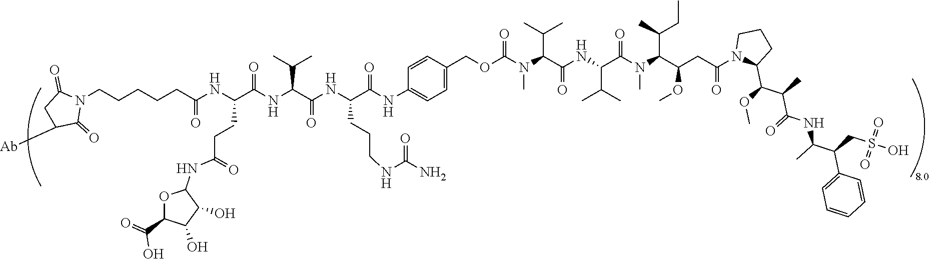 |
| ADC2-7-2 | 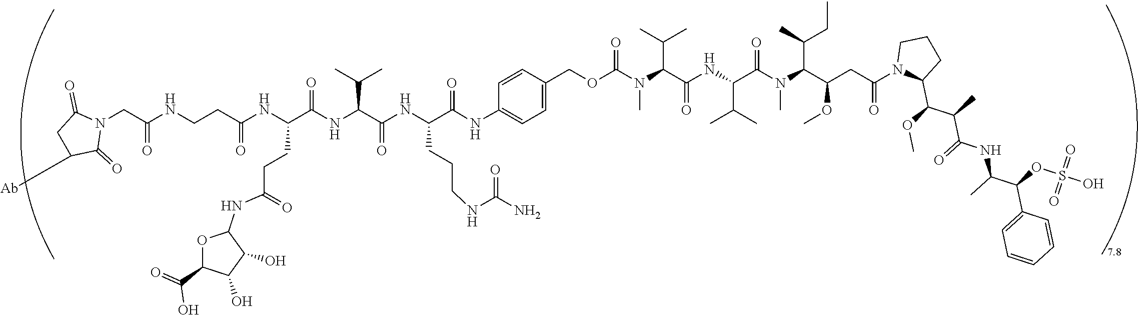 |
| ADC2-9-1 | 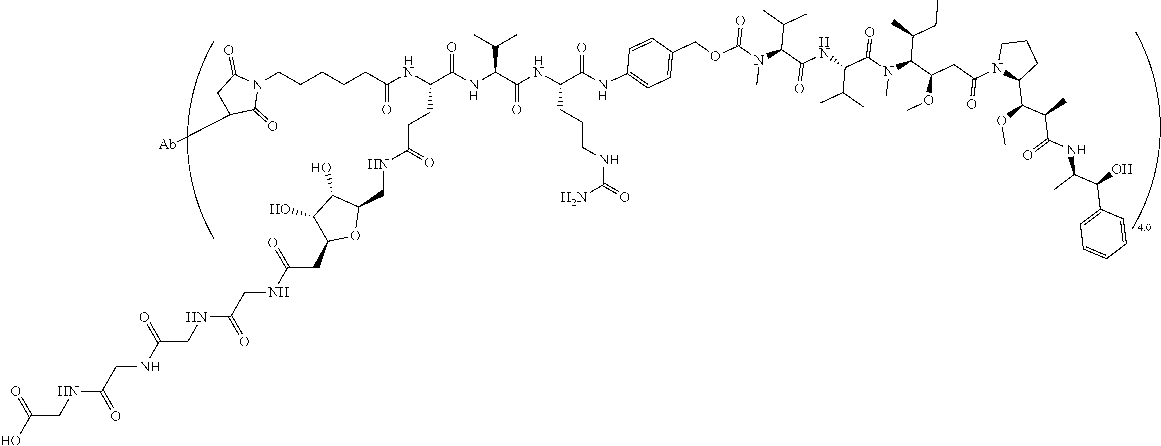 |
| ADC2-9-2 | 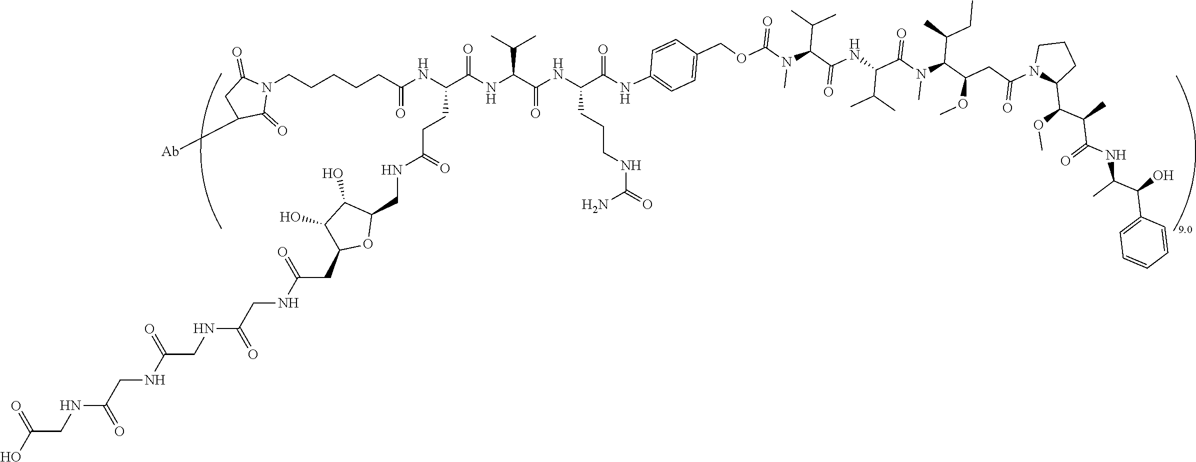 |
| ADC2-10 | 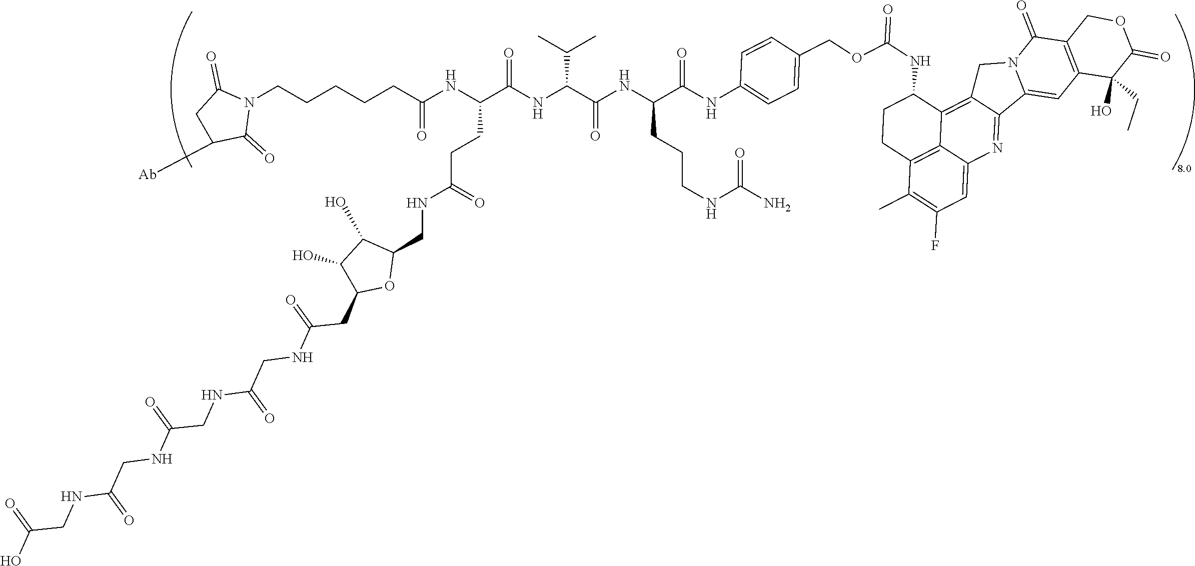 |
| ADC2-11 | 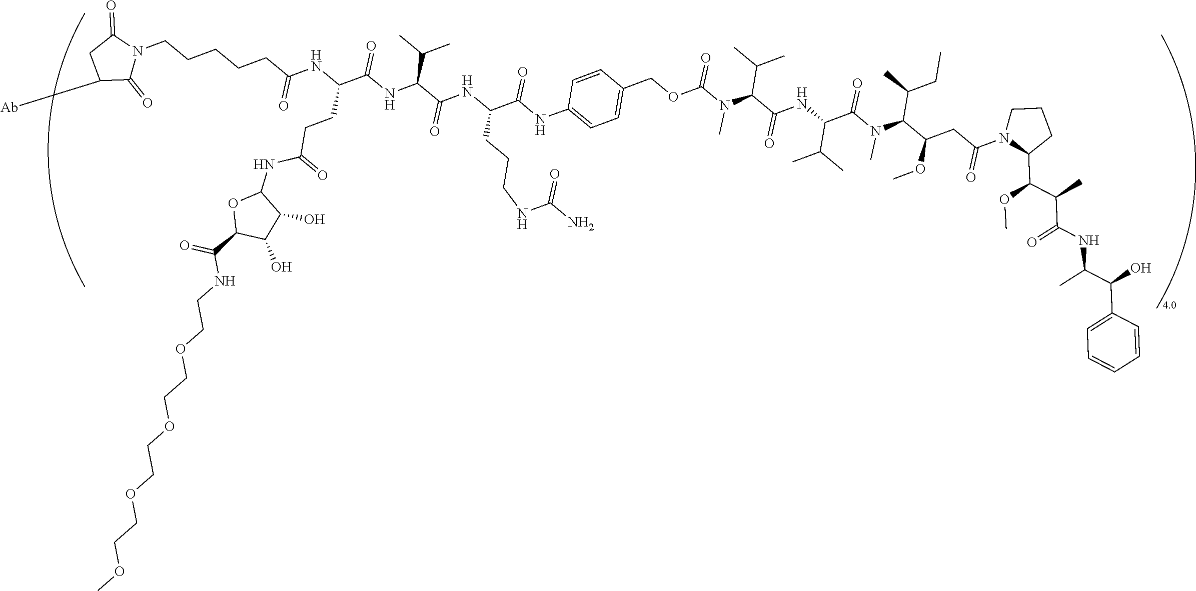 |
| ADC2-22 | 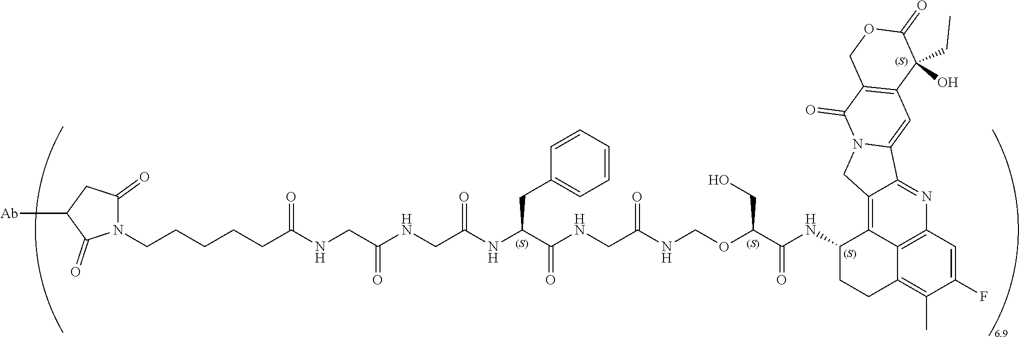 |
| ADC2-23 | 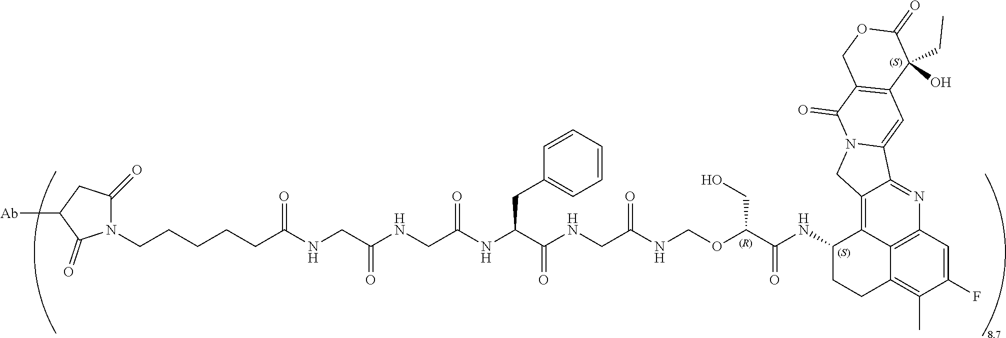 |
| ADC2-24 | 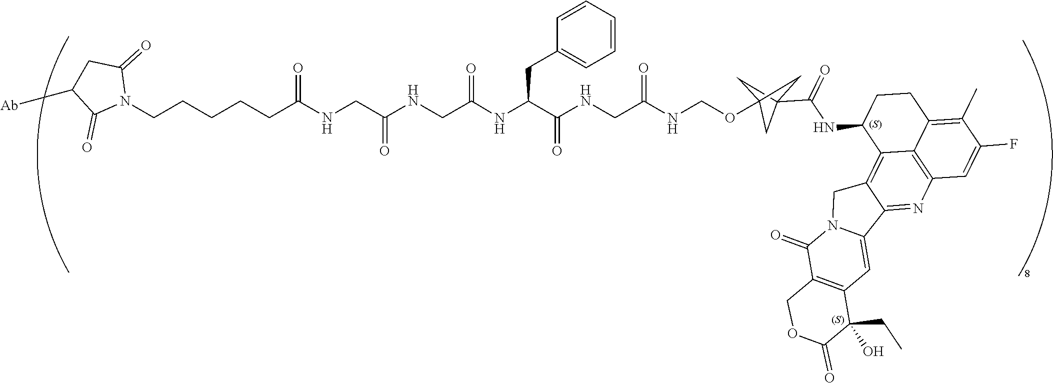 |
| ADC2-25 | 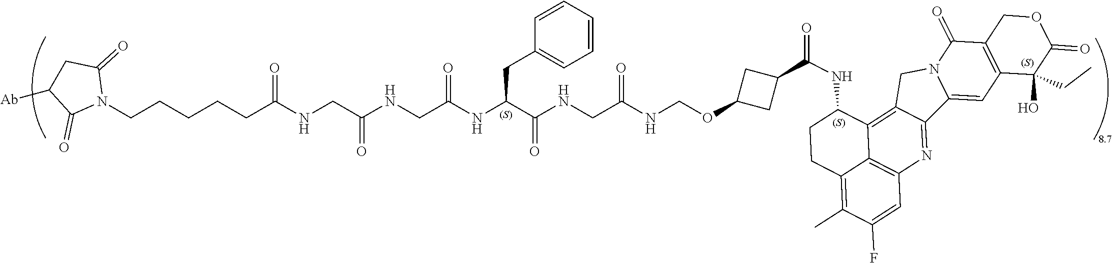 |
| ADC2-26 | 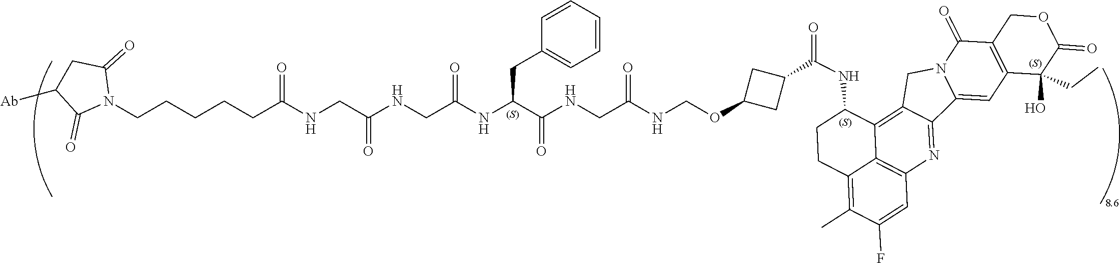 |
| ADC2-27 | 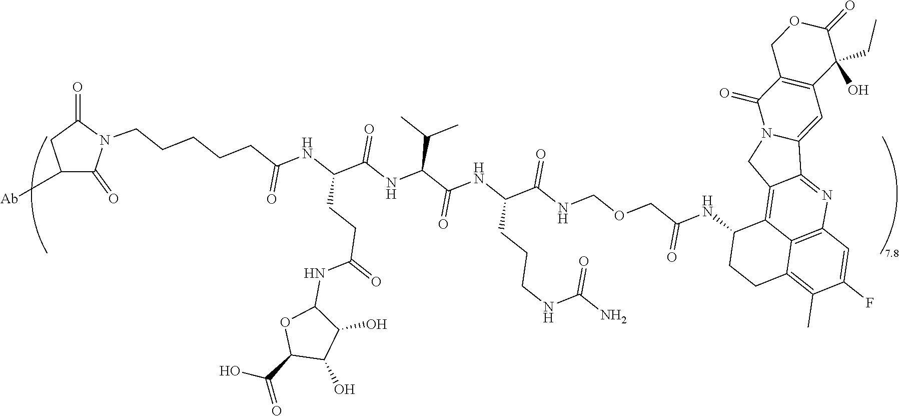 |
| ADC2-29 | 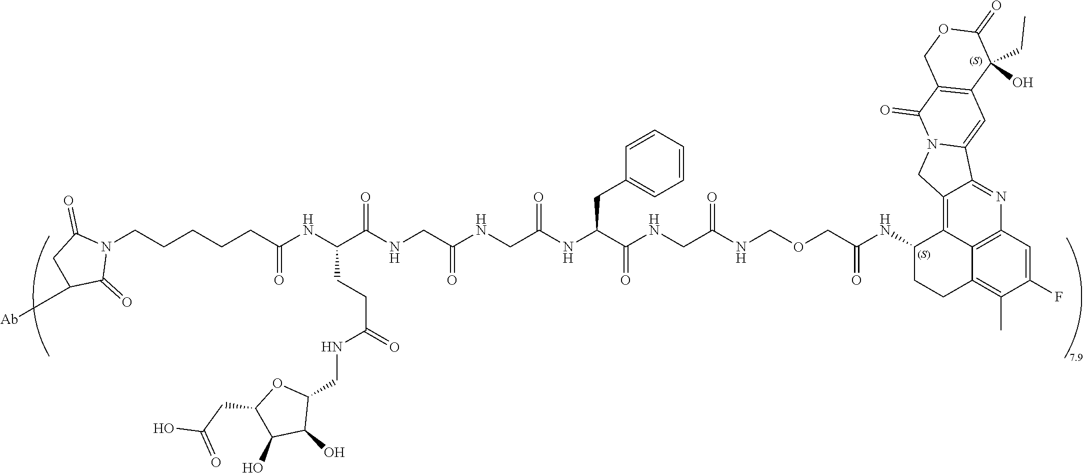 |
| ADC2-30 | 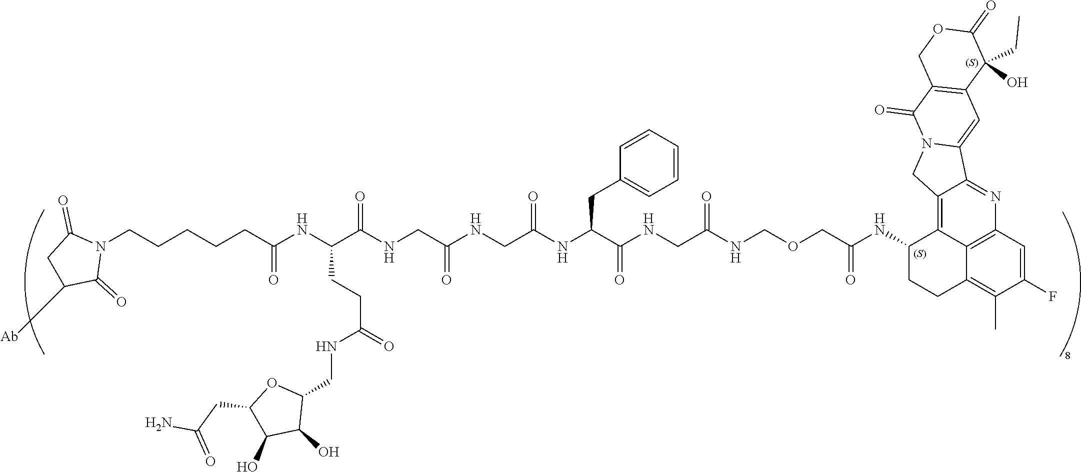 |
| ADC2-31 | 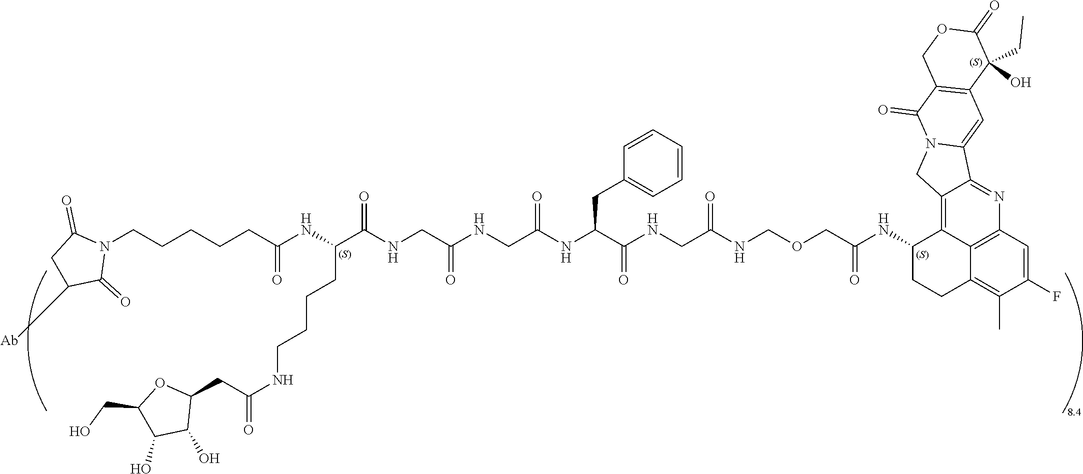 |
| ADC2-32 | 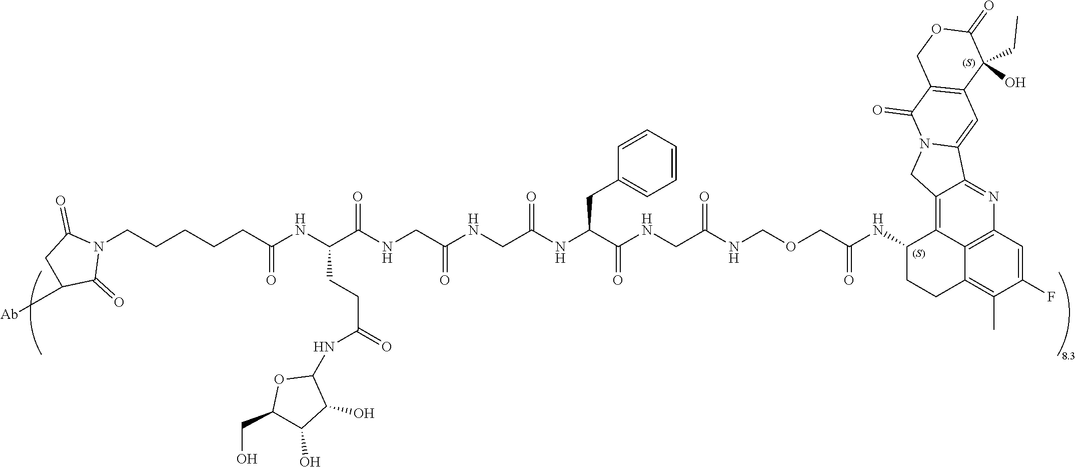 |
| ADC2-33 | 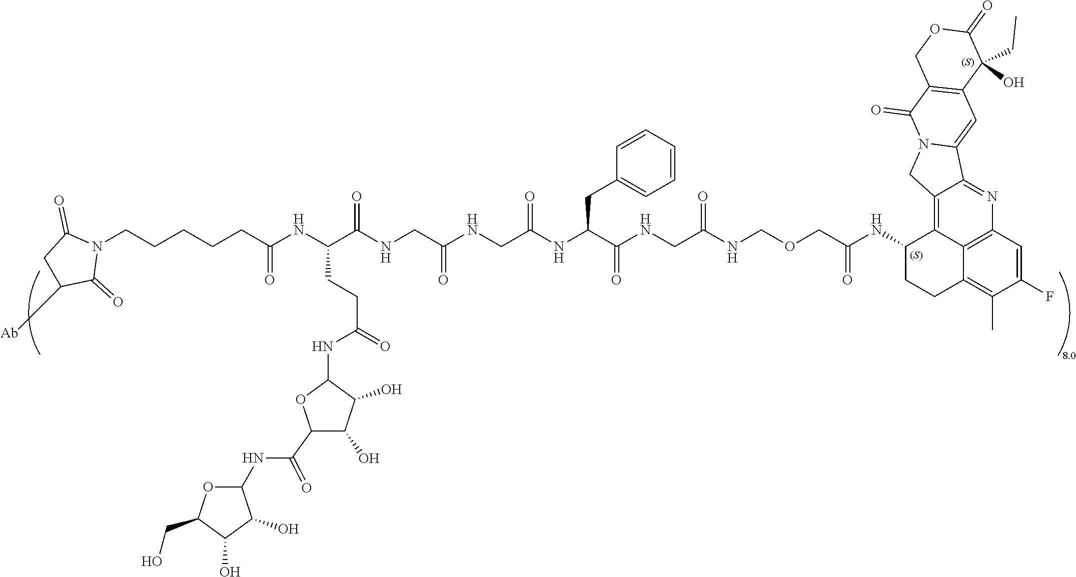 |
| ADC2-34 | 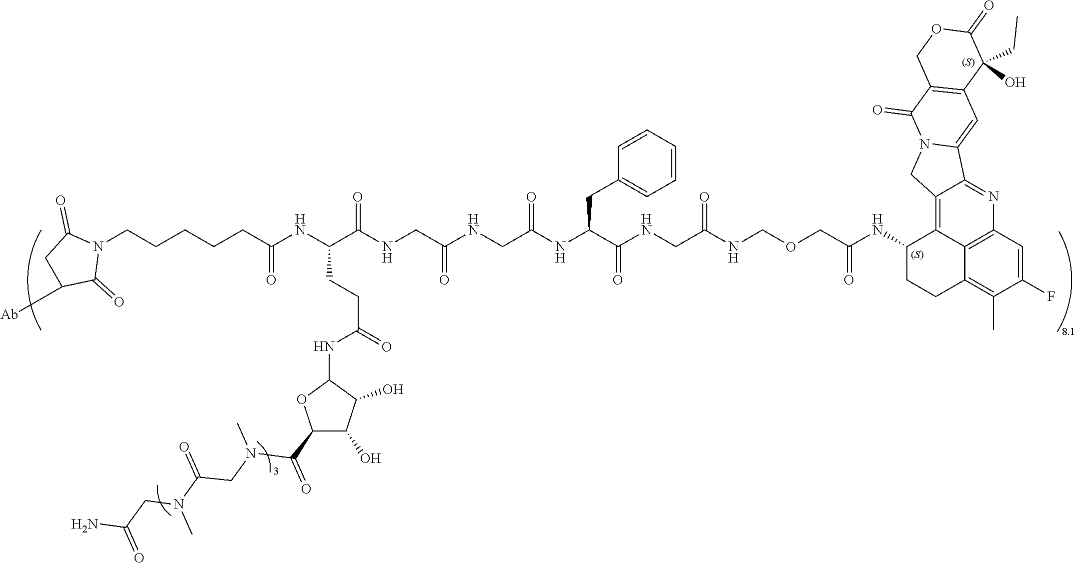 |
| ADC2-35 | 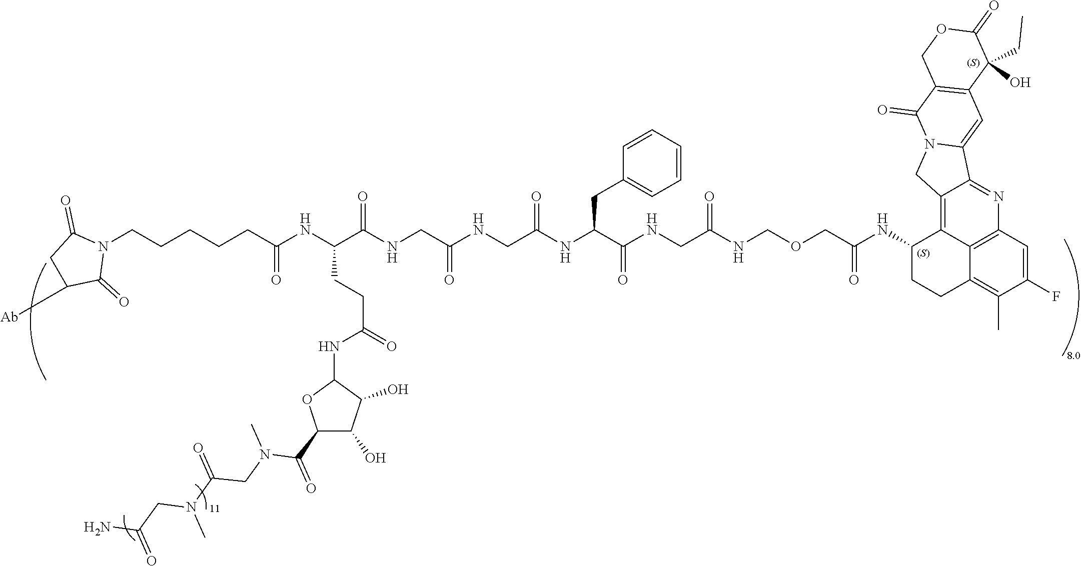 |
| ADC2-36-1 | 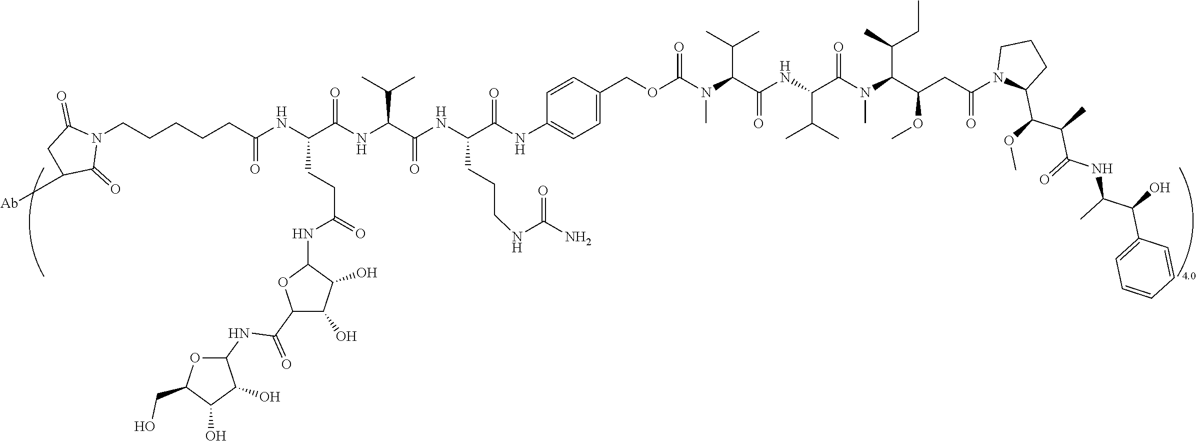 |
| ADC2-36-2 | 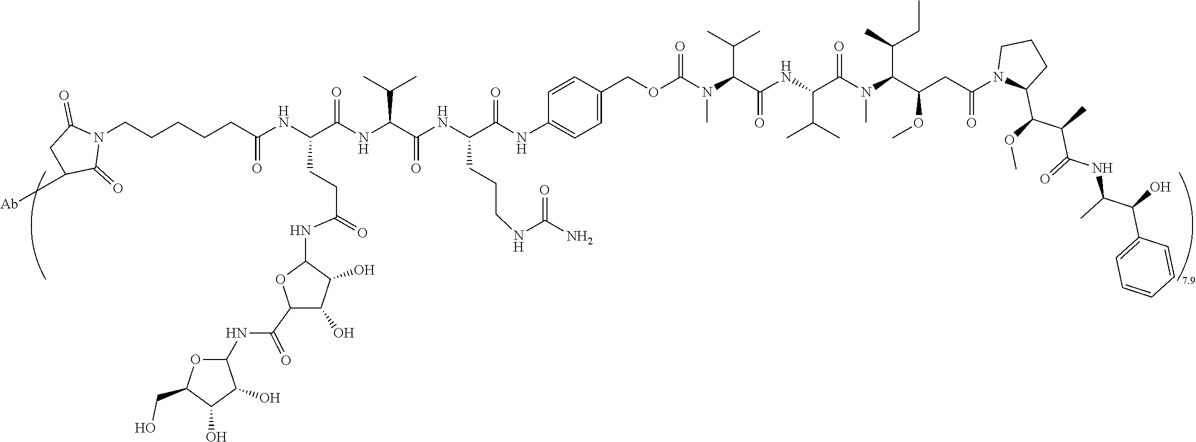 |
| ADC2-37 | 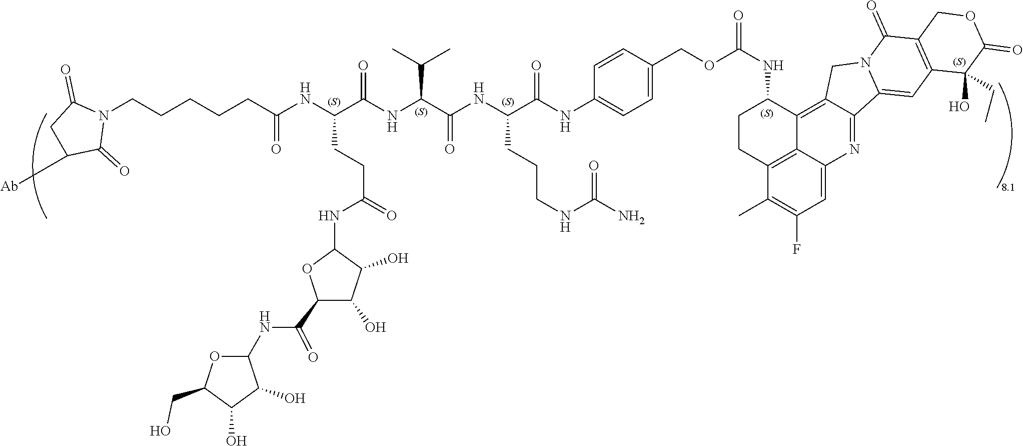 |
| ADC2-38 | 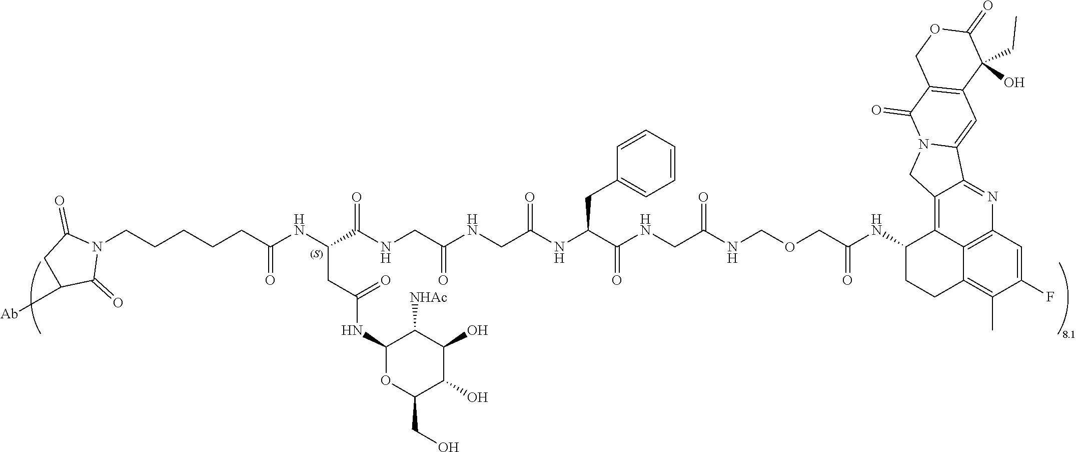 |
| ADC2-39 | 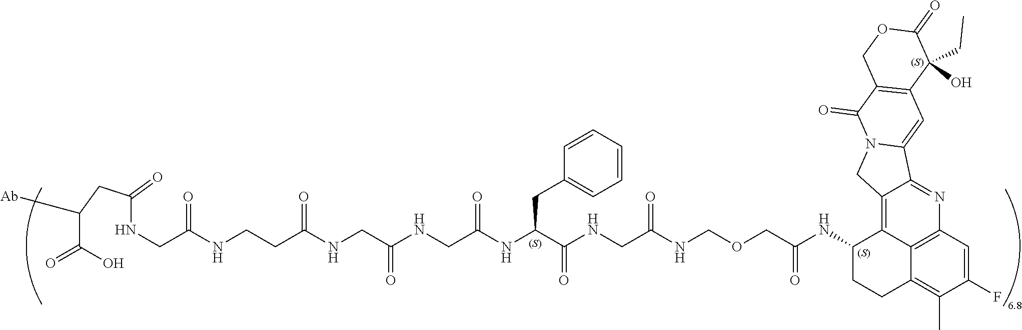 |
| ADC2-40 | 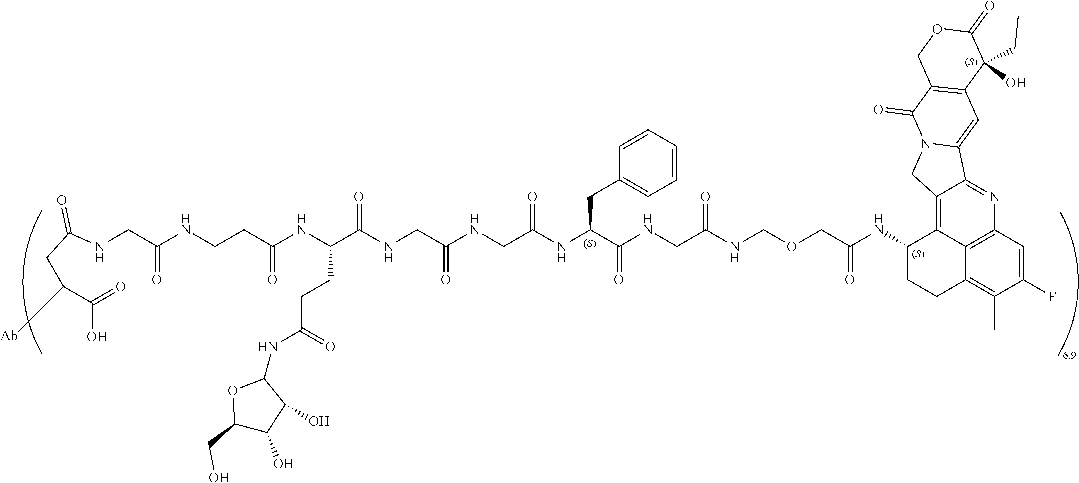 |
| ADC2-41 | 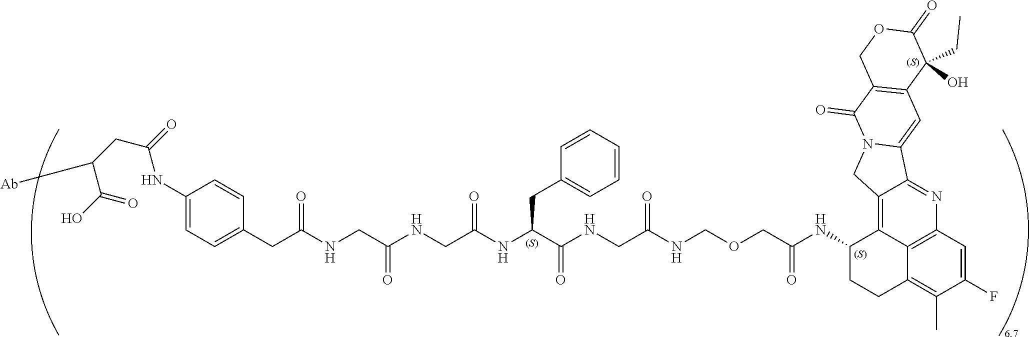 |
| ADC2-42 | 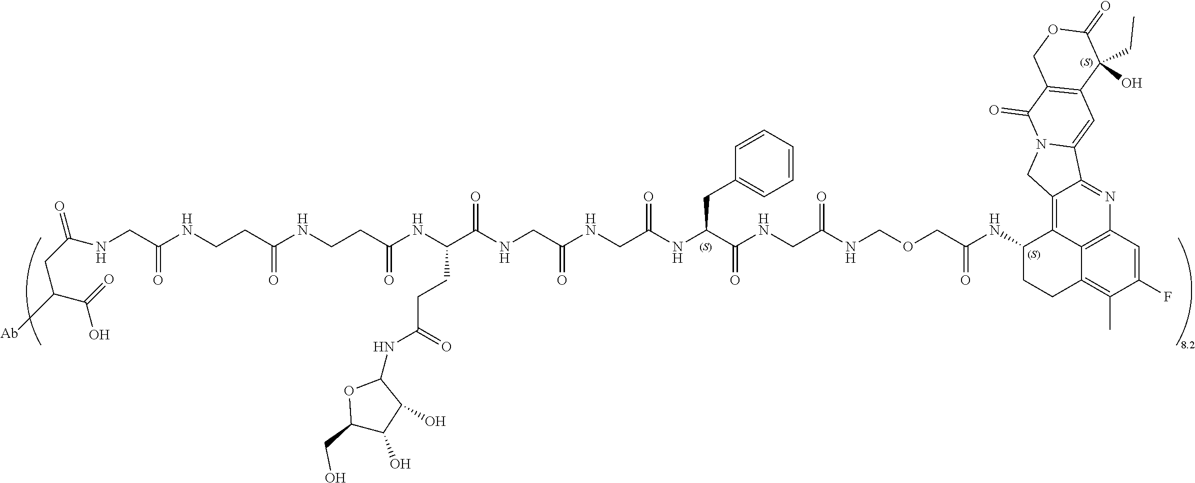 |
| ADC2-42-RO | 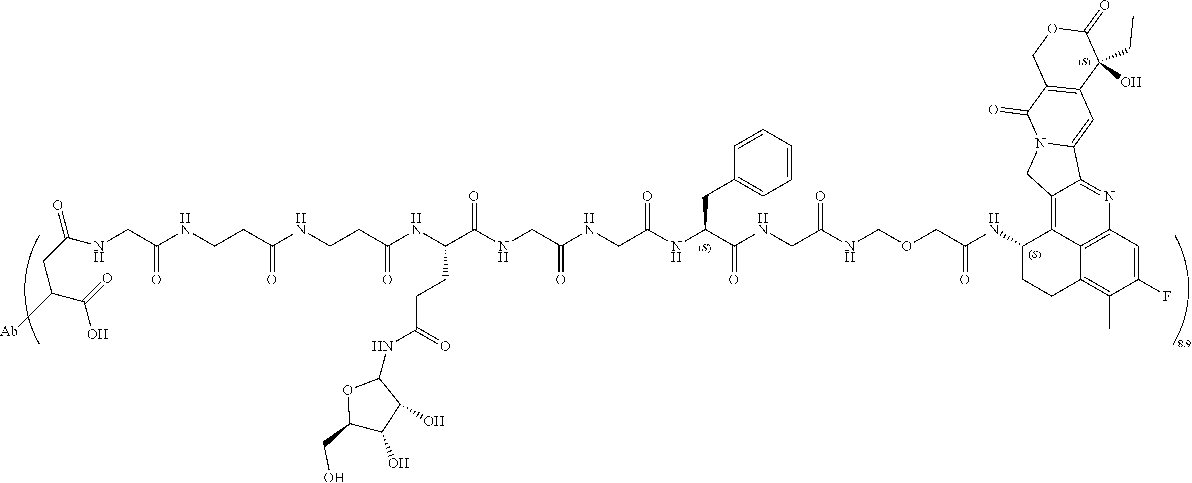 |
| ADC2-43 | 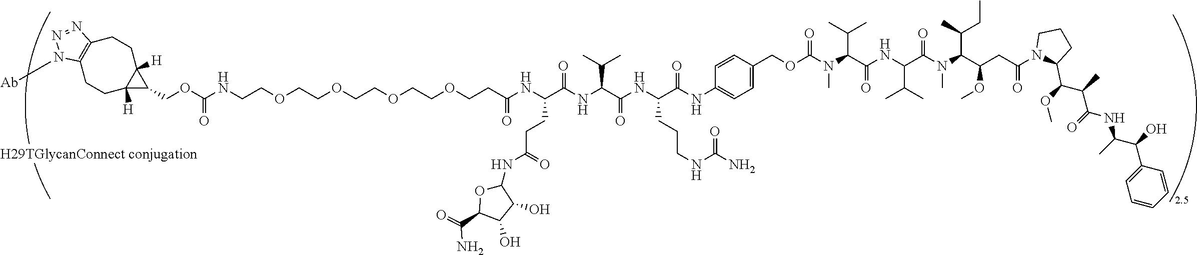 |
| ADC2-44 | 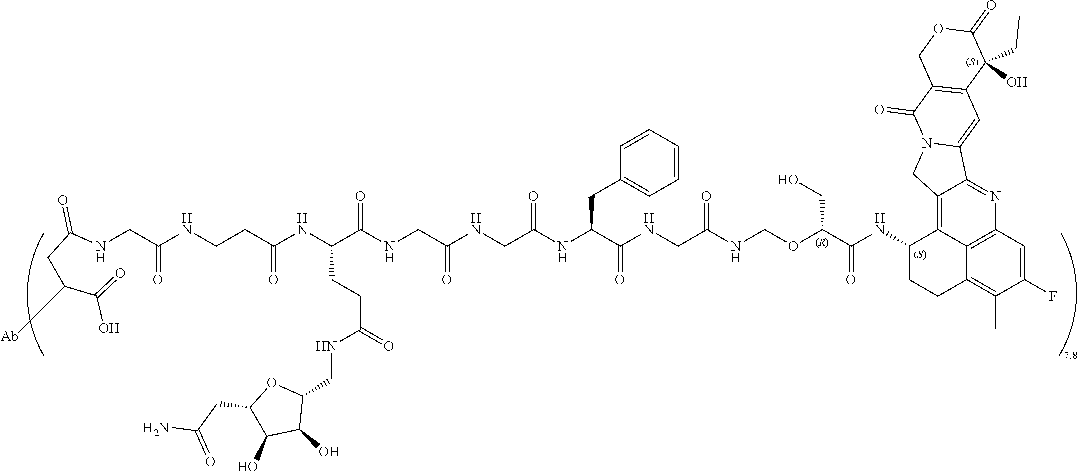 |
| ADC2-45 | 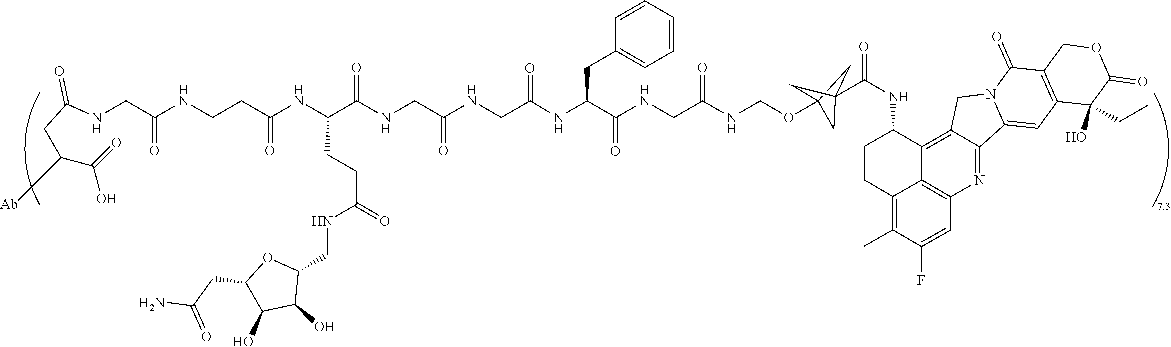 |
| ADC2-46 | 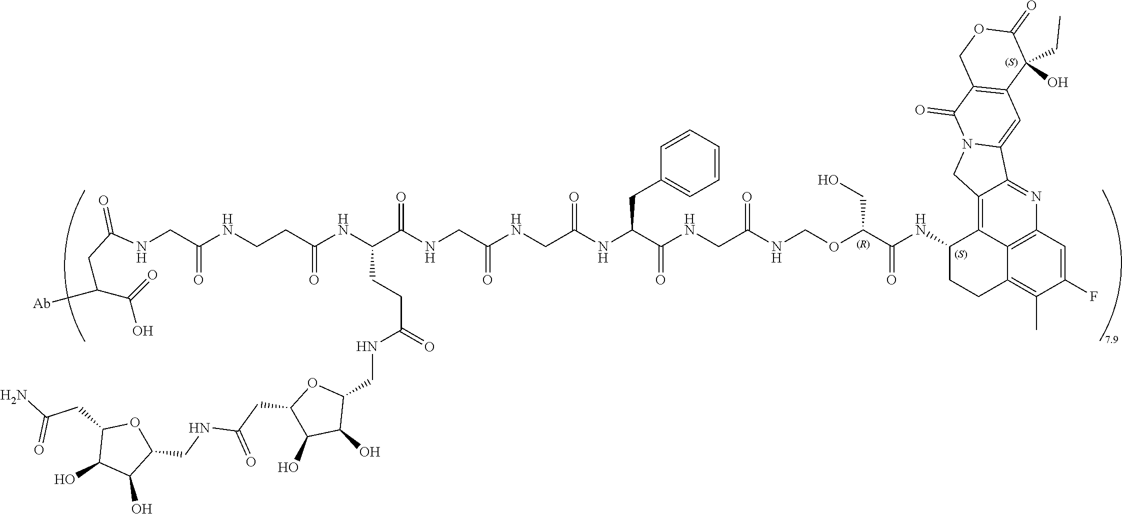 |
| ADC2-47 | 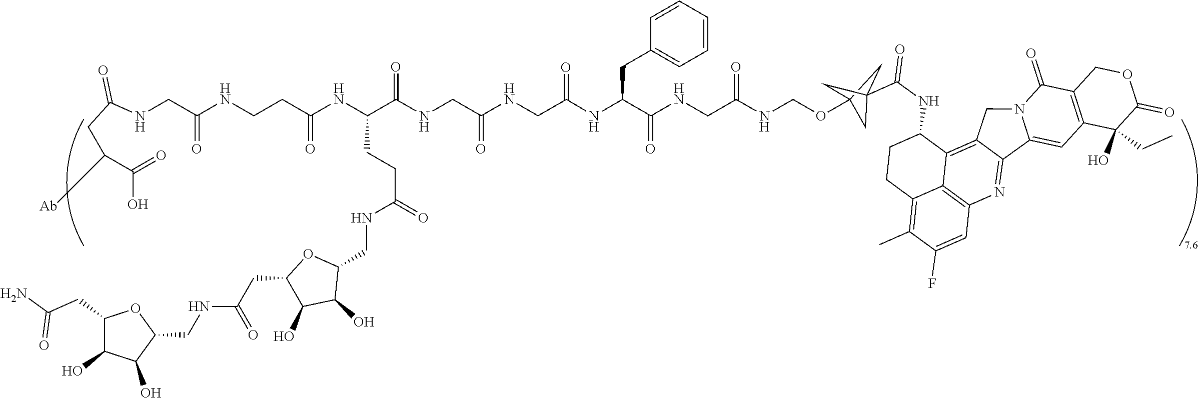 |
| ADC2-48 | 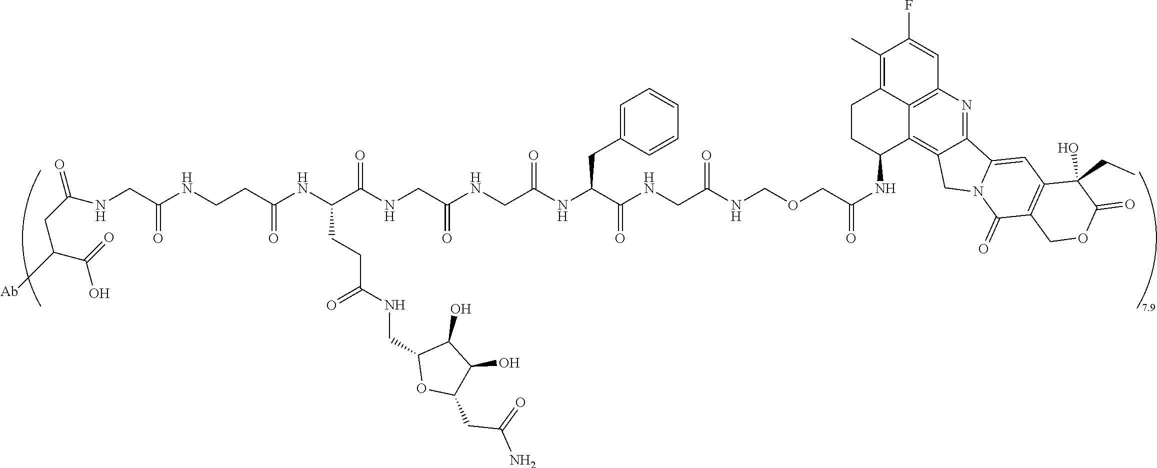 |
| ADC2-49 | 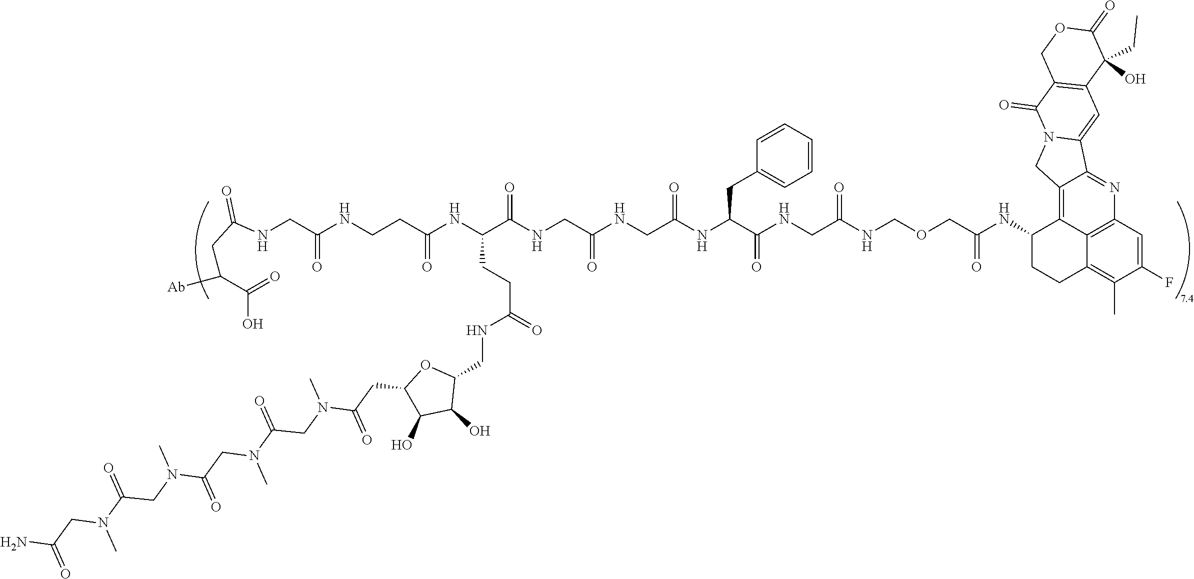 |
| ADC2-50 | 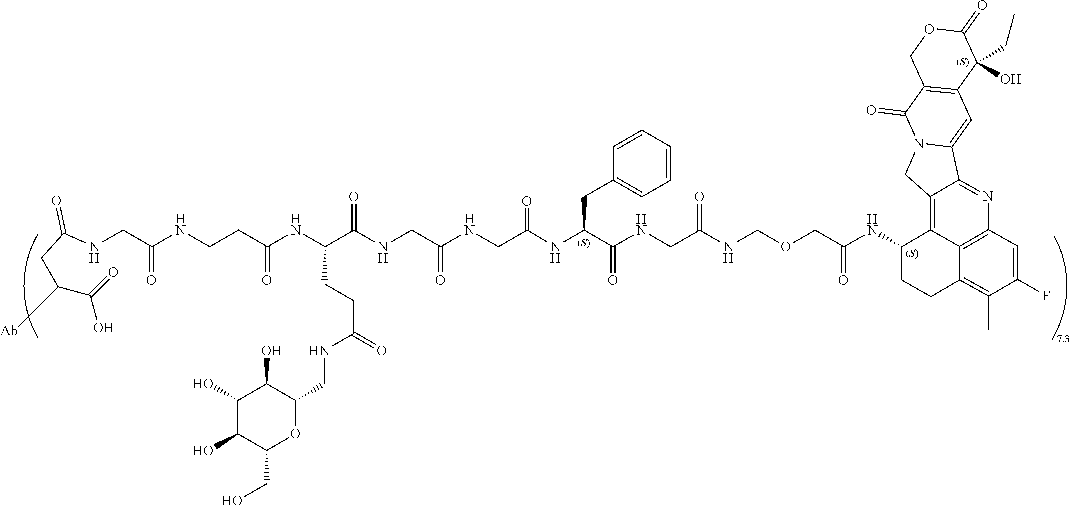 |
| ADC2-51 | 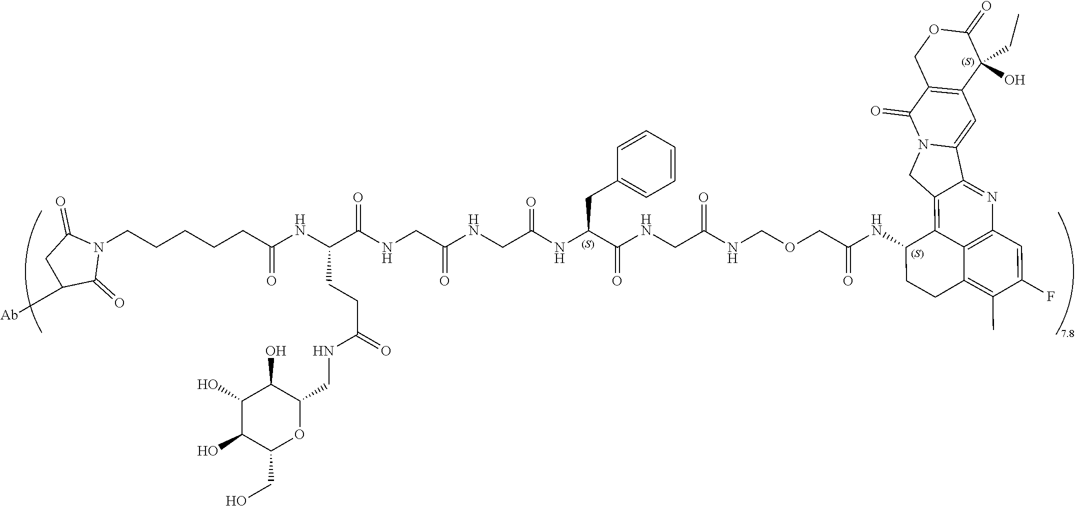 |
| ADC2-53 | 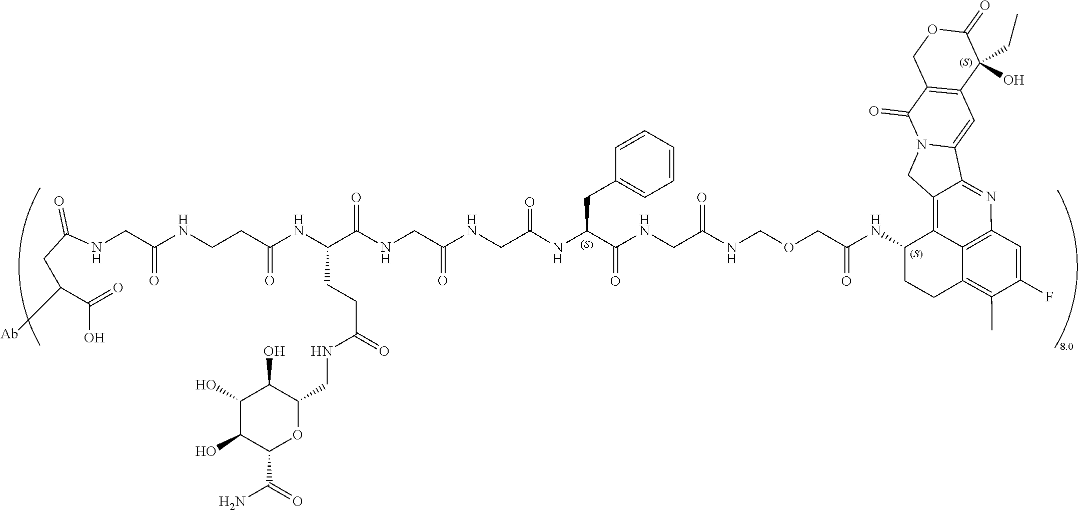 |
| ADC2-54 | 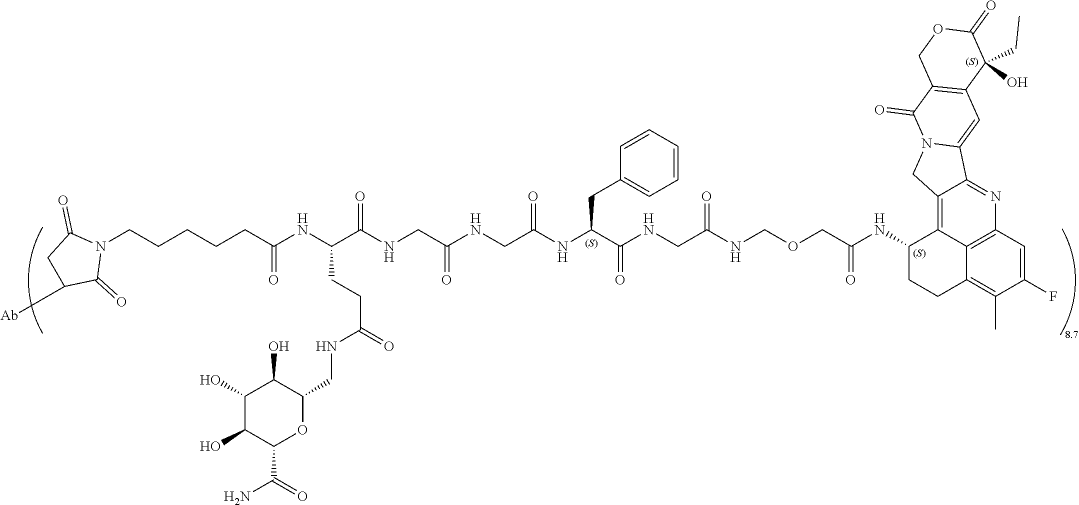 |
| ADC2-55 | 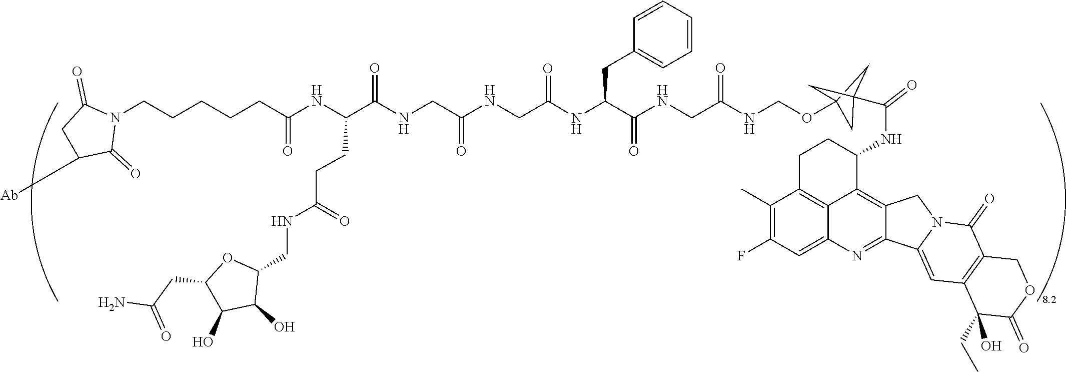 |
| ADC2-56 | 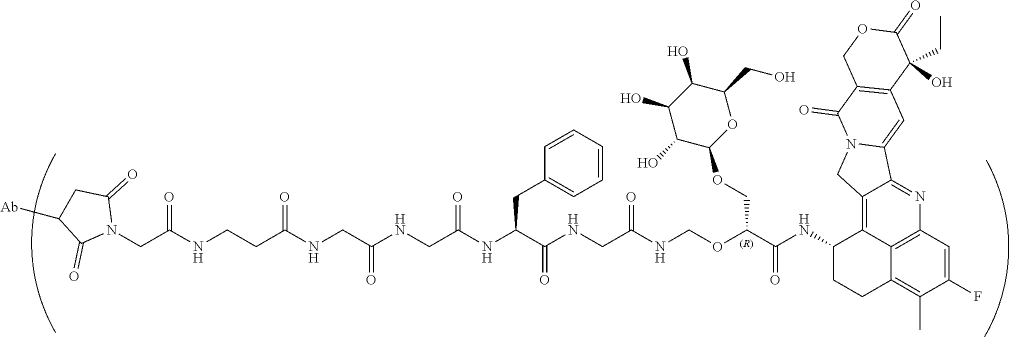 |
| ADC2-57 | 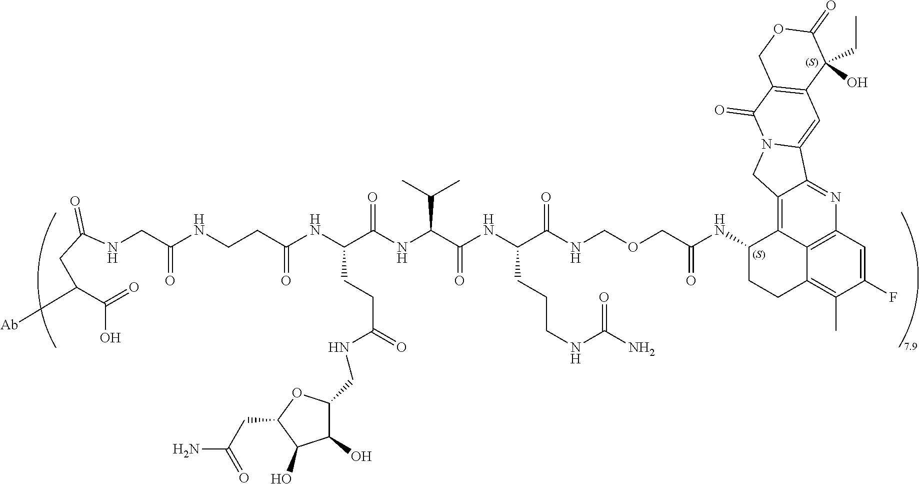 |
| ADC2-58 | 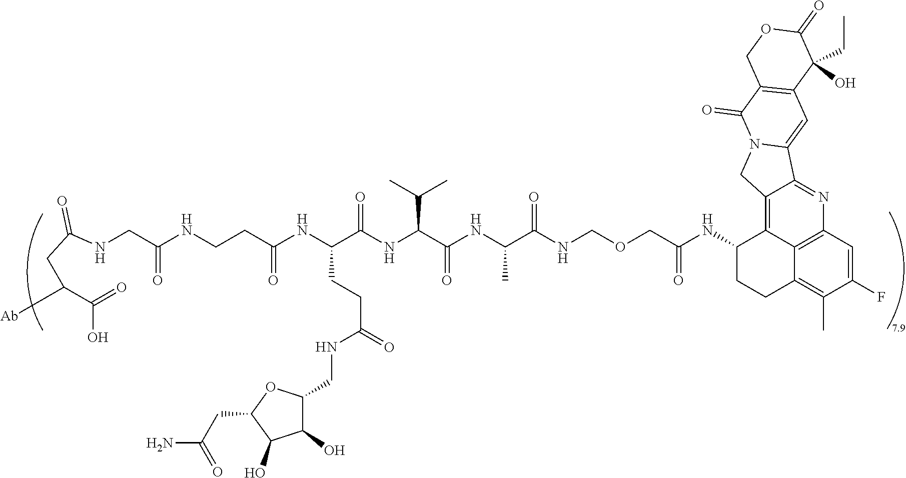 |
| ADC2-59 | 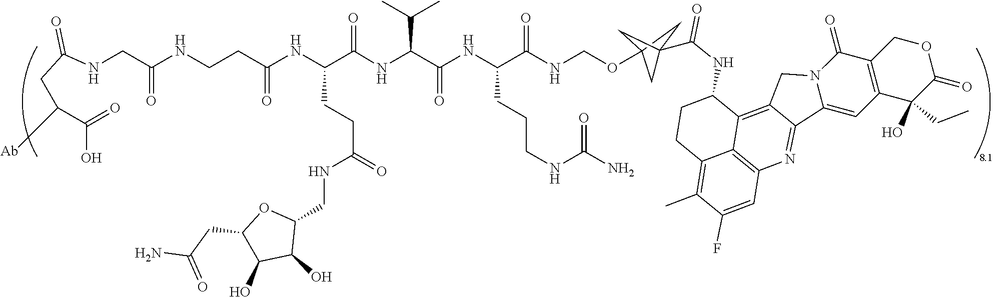 |
| ADC2-60 | 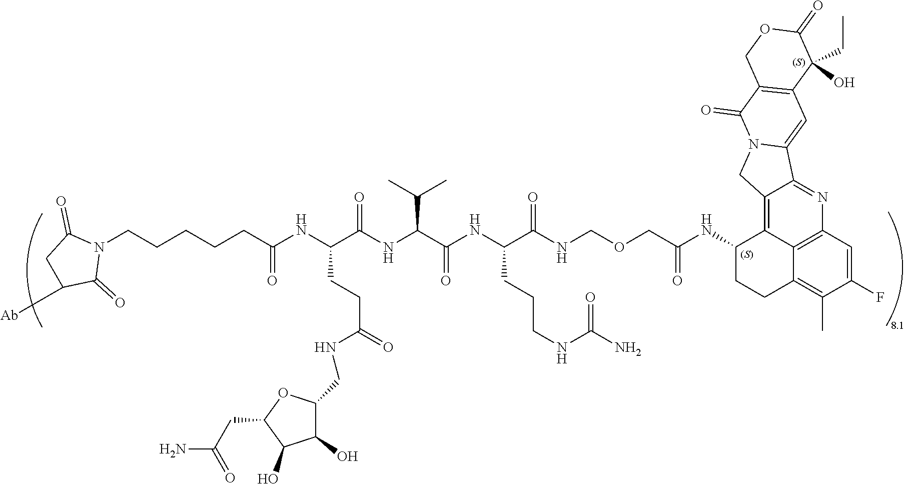 |
| ADC2-61 | 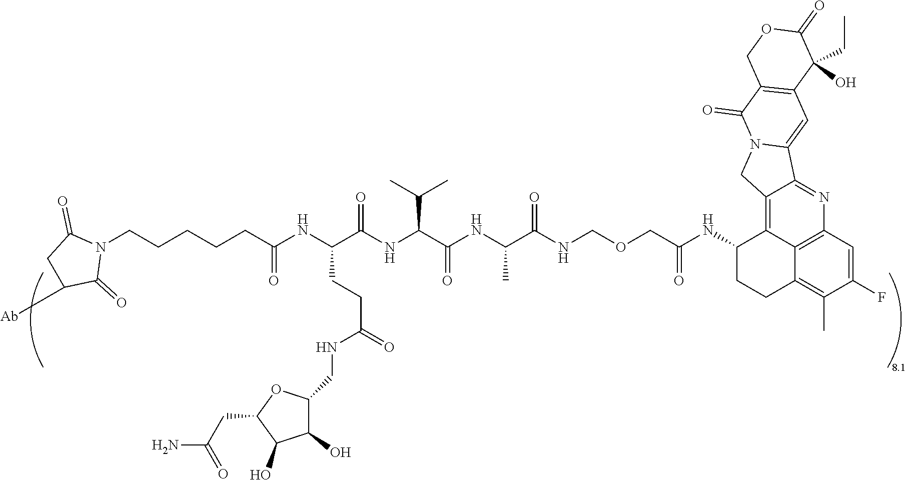 |
| ADC2-62-1 | 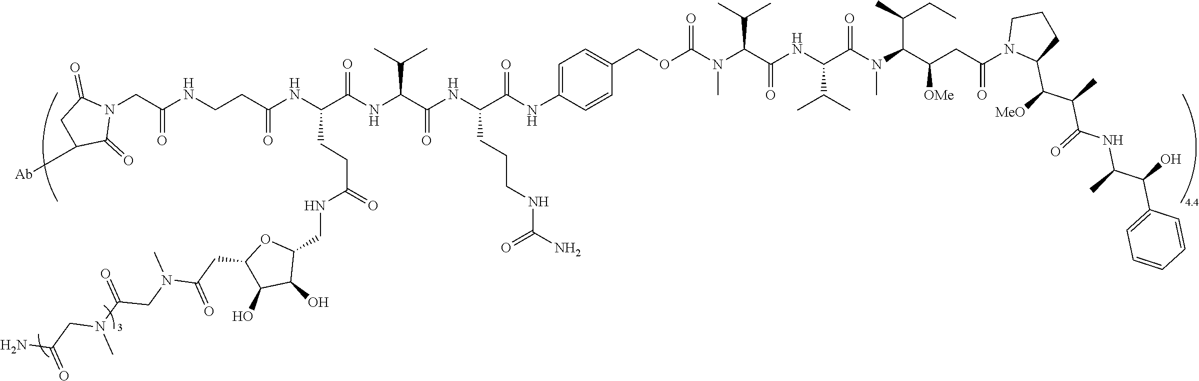 |
| ADC2-62-2 | 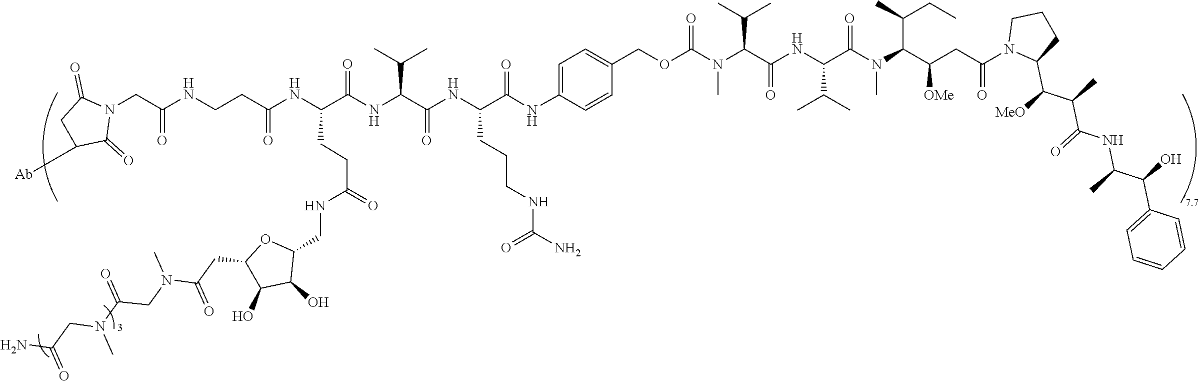 |
| ADC2-63-1 | 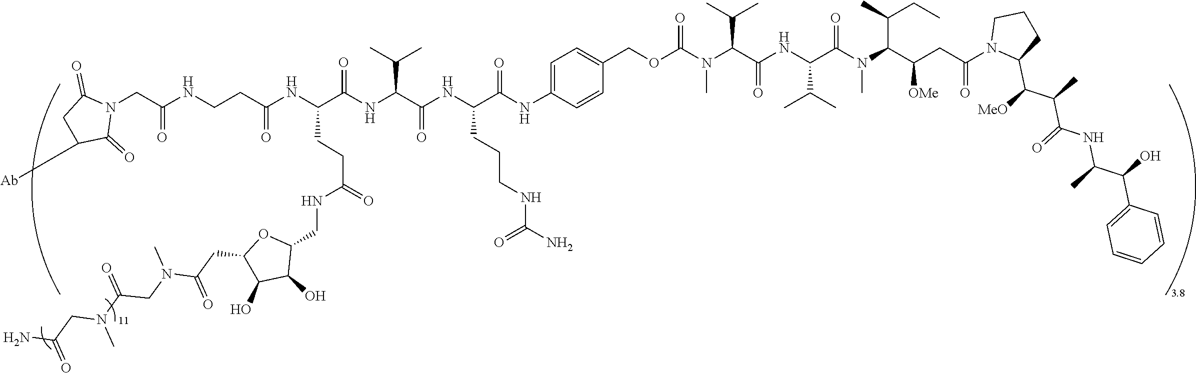 |
| ADC2-63-2 | 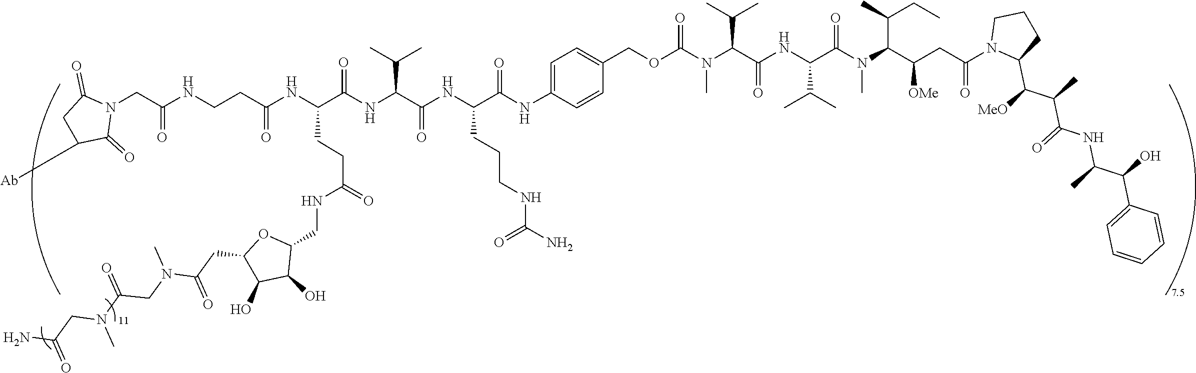 |
| ADC2-64-1 | 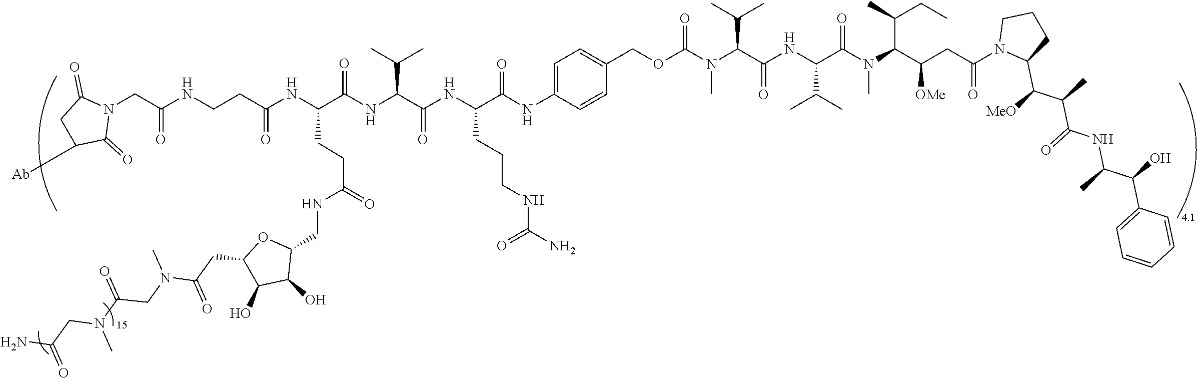 |
| ADC2-64-2 | 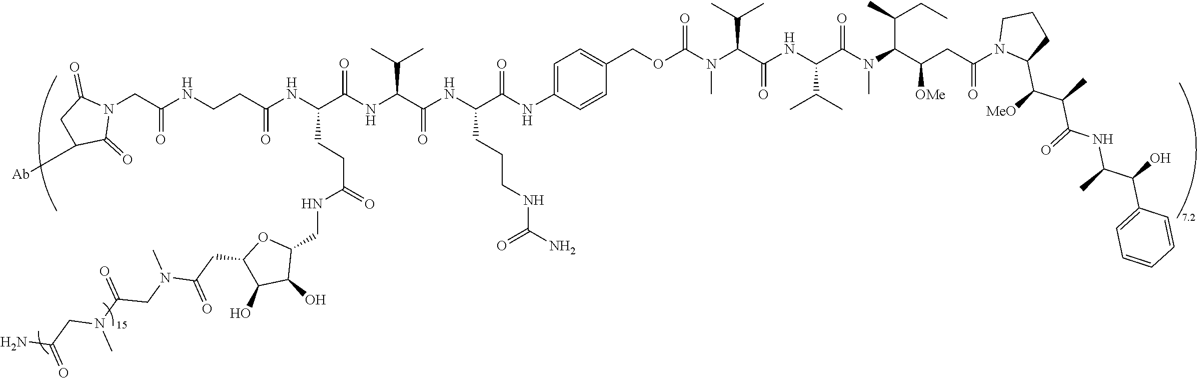 |
| ADC2-65-1 | 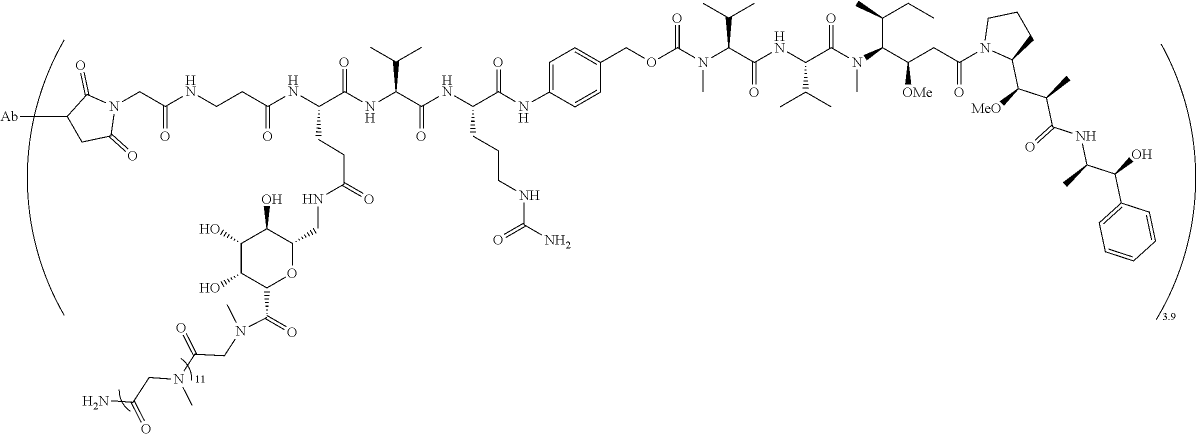 |
| ADC2-65-2 | 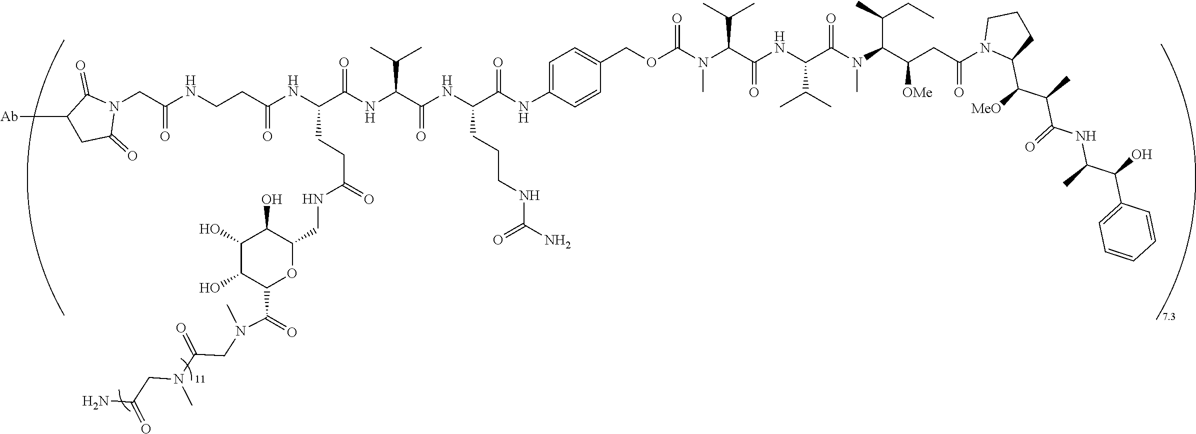 |
| ADC4-1 | 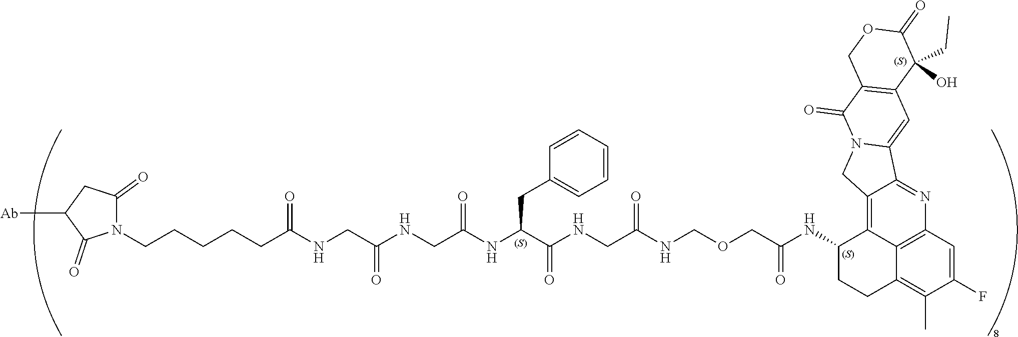 |
| ADC4-2 | 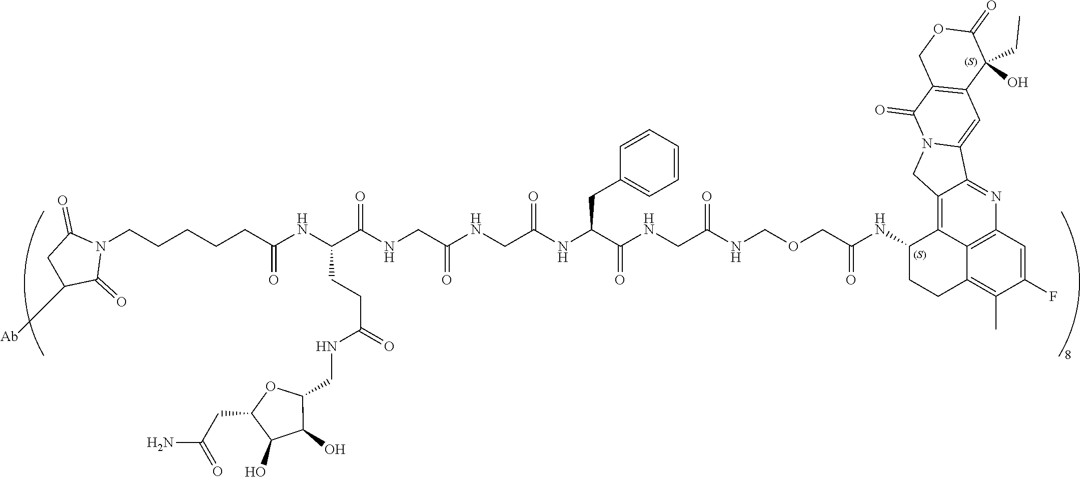 |
| ADC4-3 | 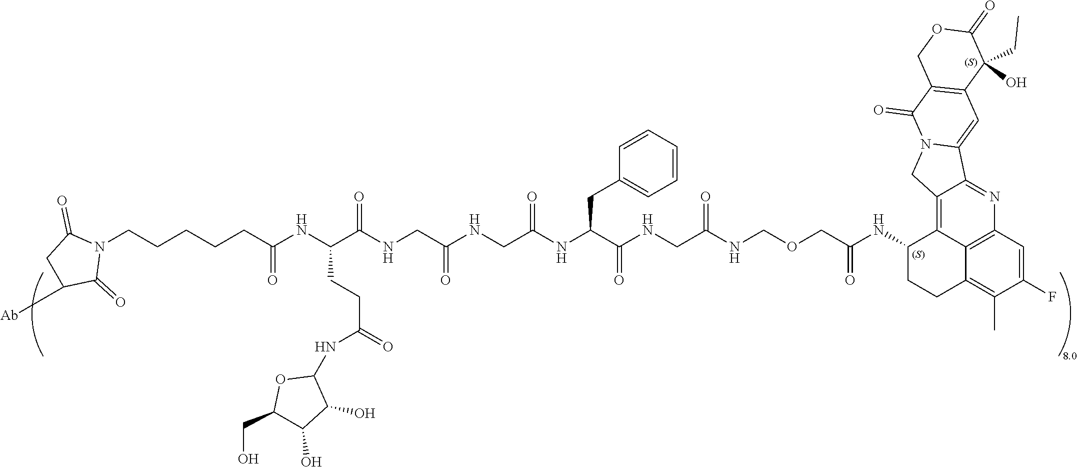 |
| ADC4-4 | 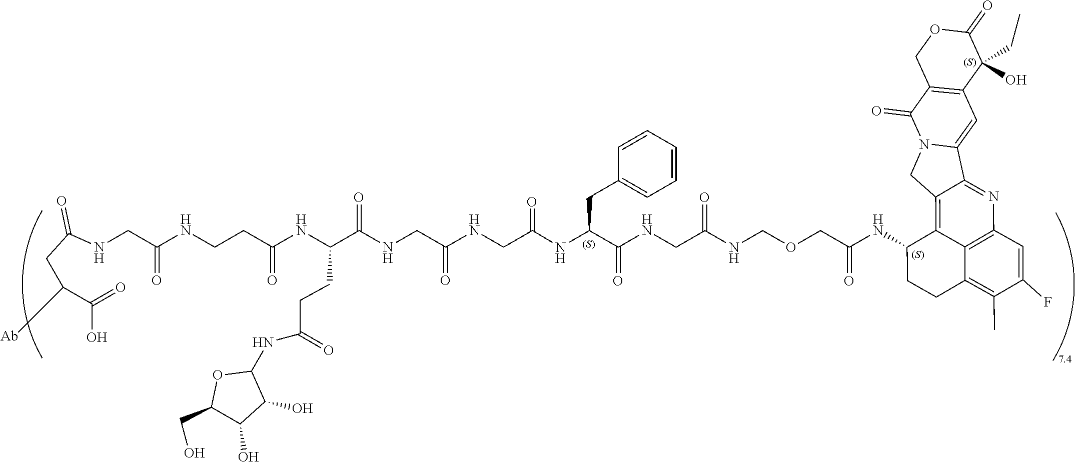 |
| ADC4-5 | 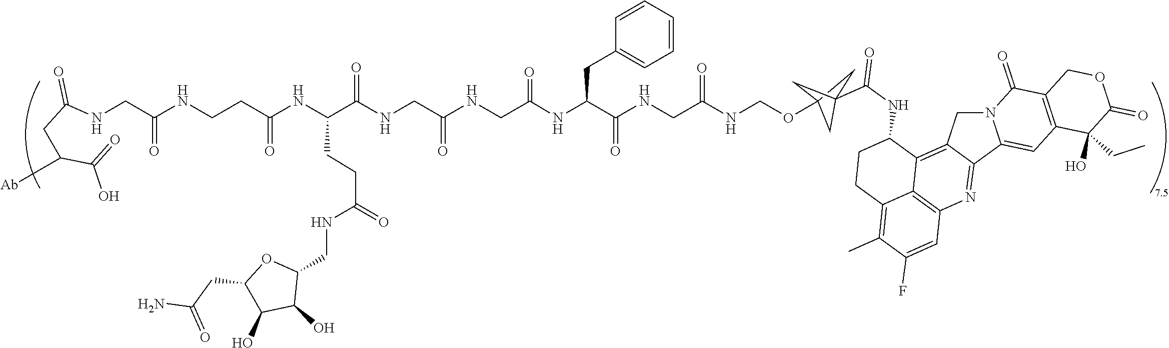 |
| ADC4-6 | 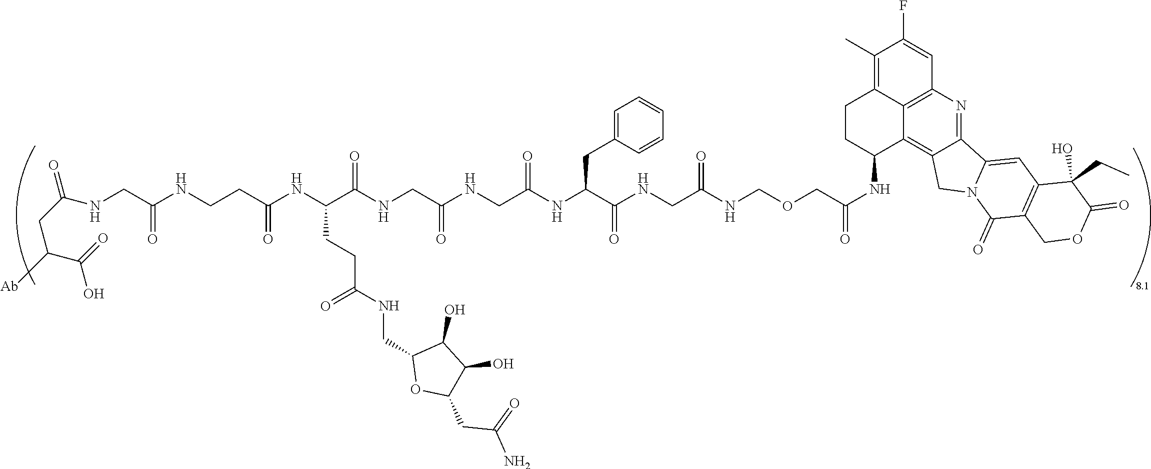 |
| ADC4-7 | 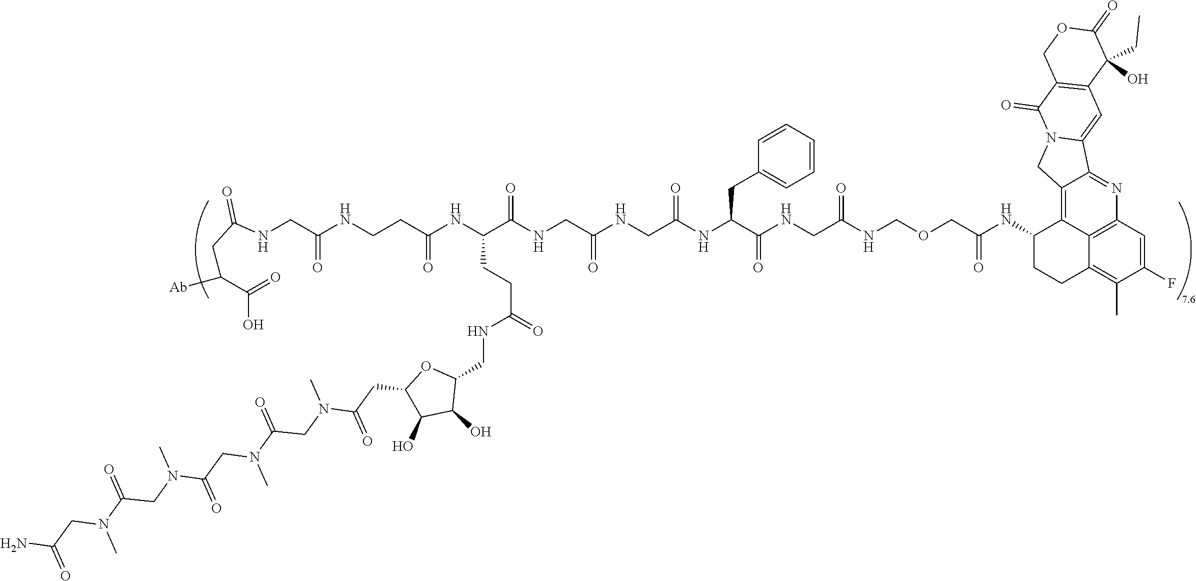 |
| ADC4-8 | 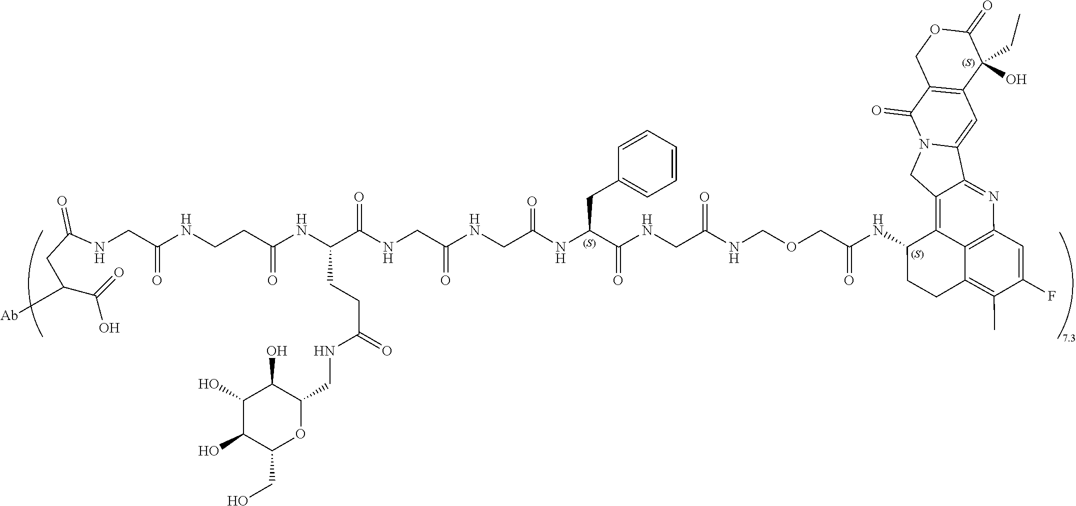 |
| ADC4-9 | 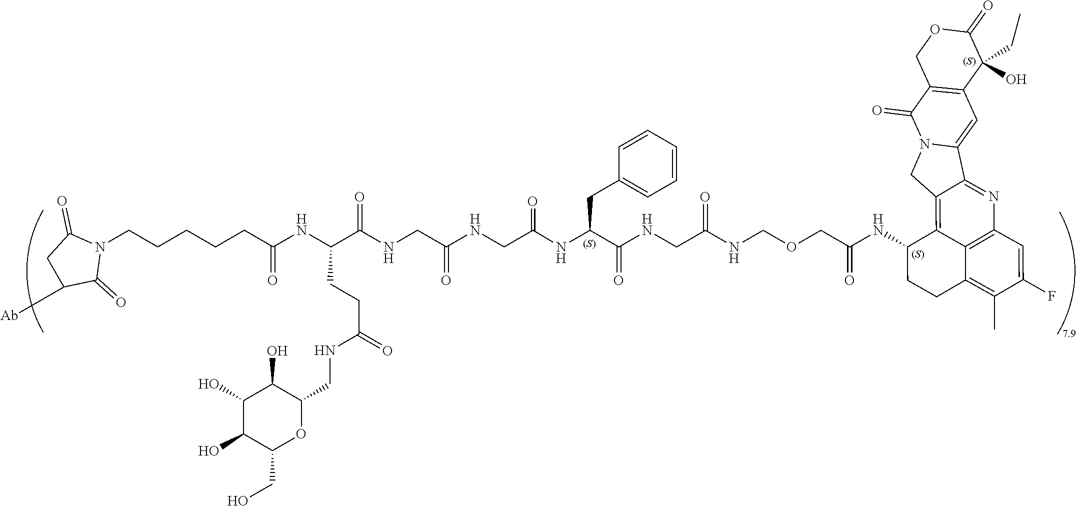 |
| ADC4-10 | 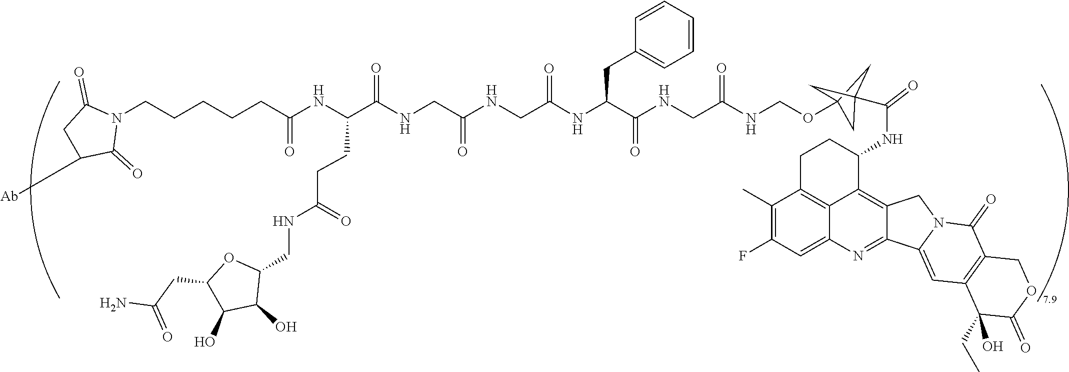 |
| ADC4-11 | 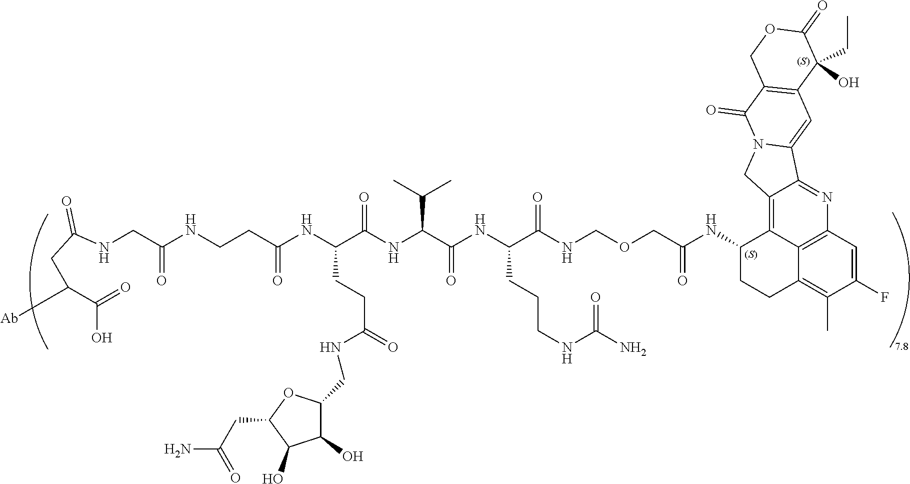 |
| ADC4-12 | 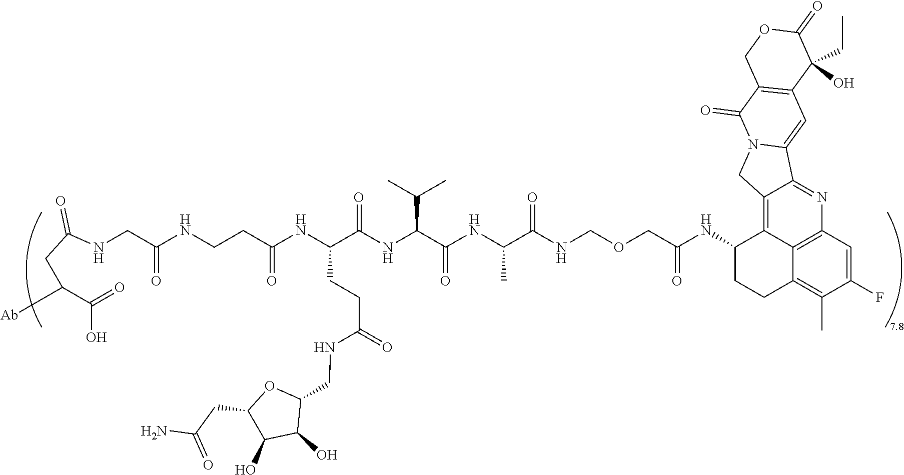 |
| ADC4-13 | 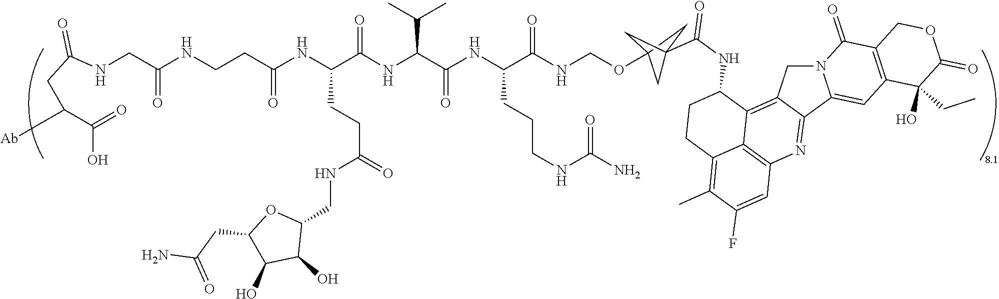 |
| ADC4-14 | 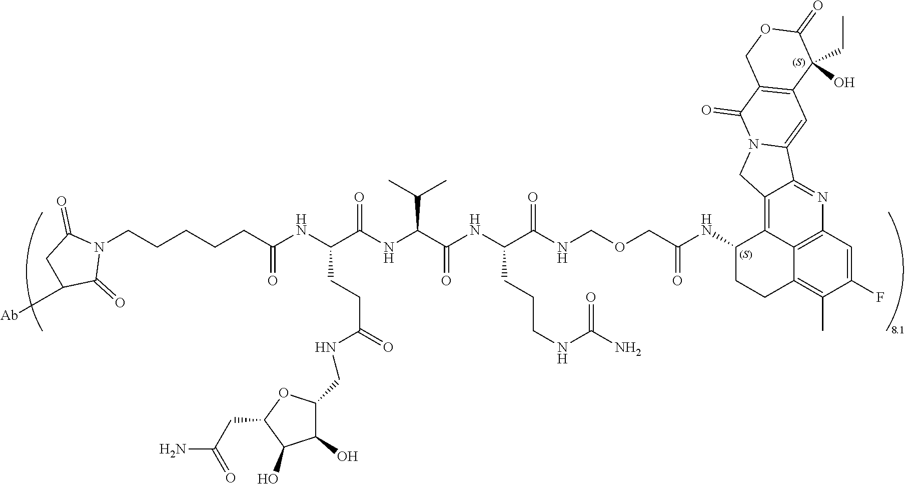 |
| ADC4-15 | 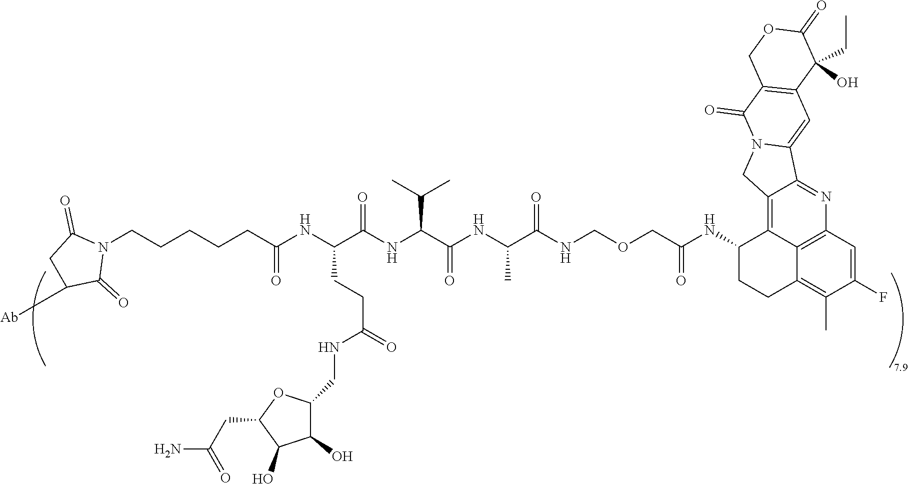 |
| ADC4-16 | 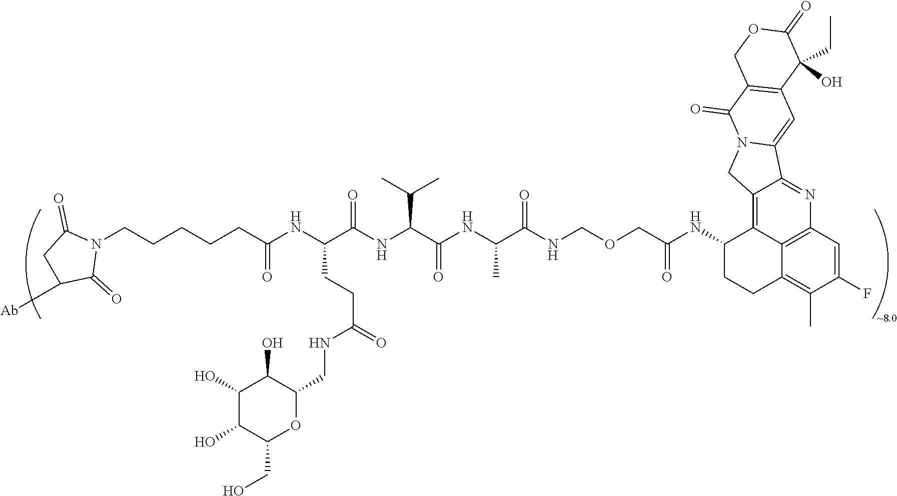 |
| ADC4-17 | 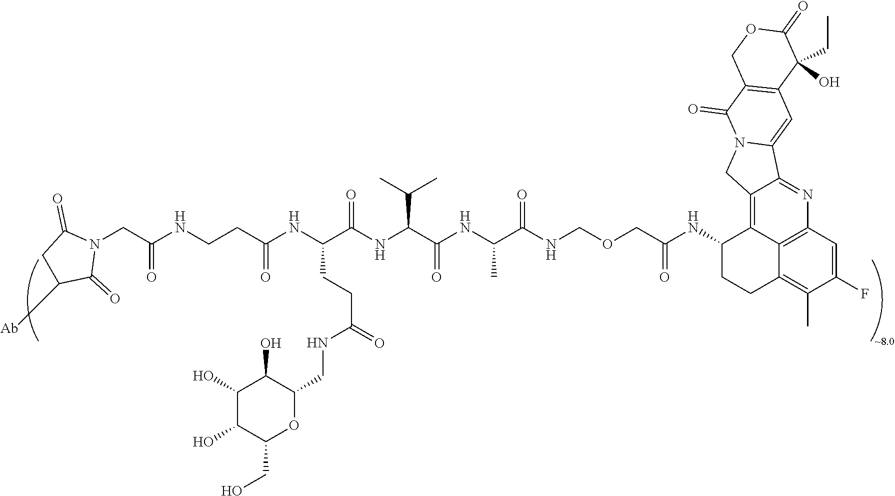 |
| ADC4-18 | 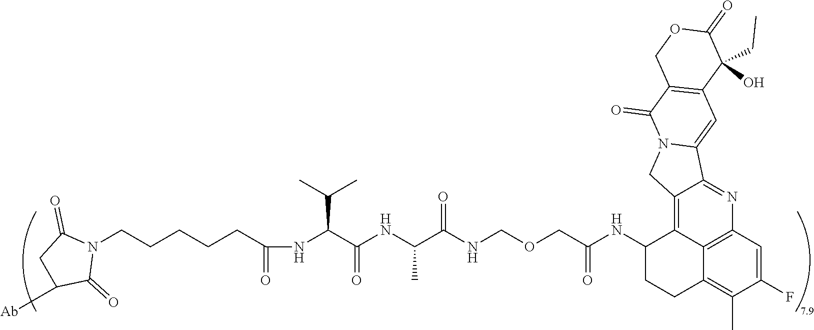 |
| ADC4-19 | 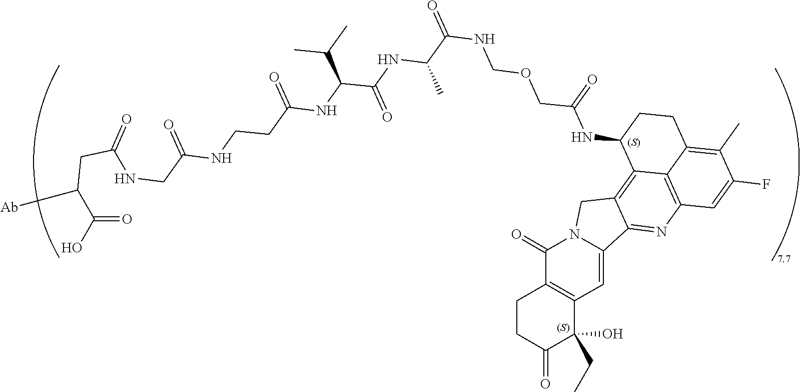 |
| ADC4-20 | 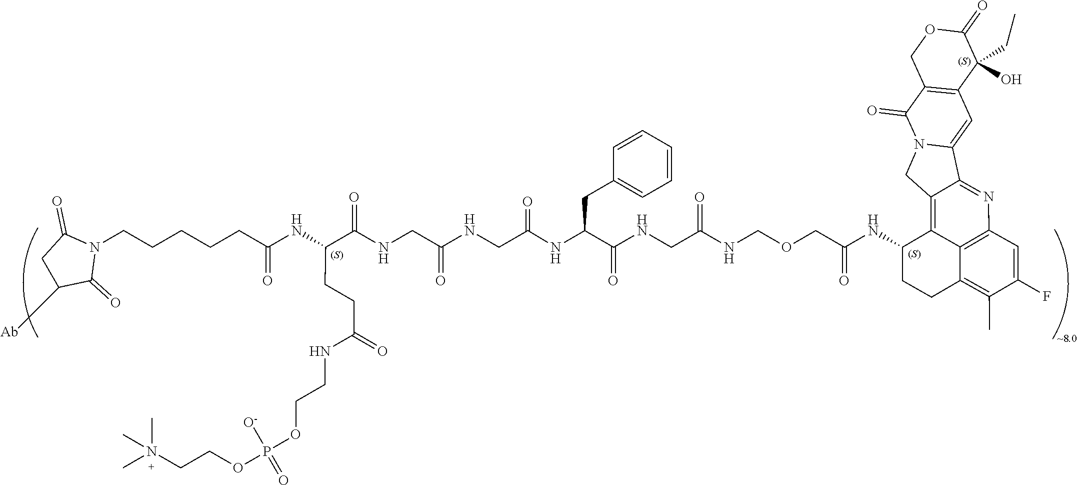 |
| ADC-C2 | 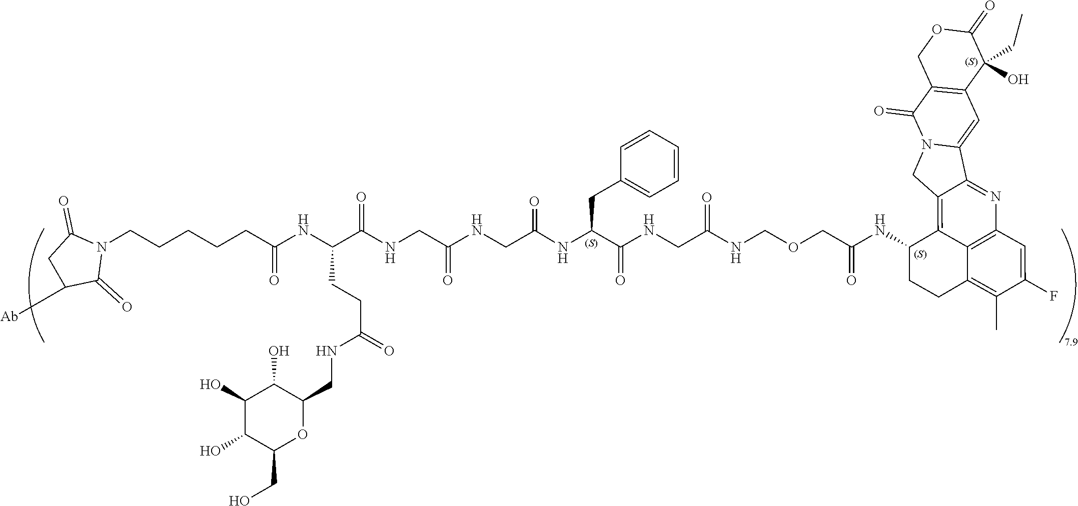 |
| ADC-C3 | 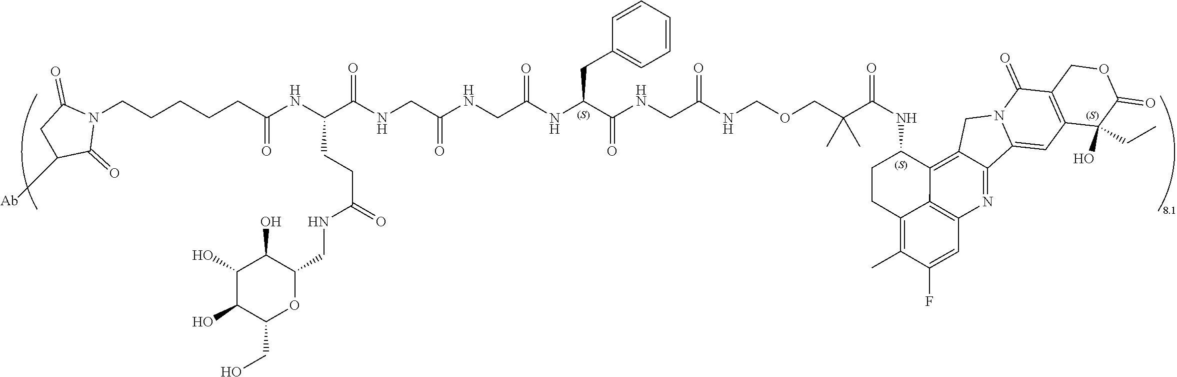 |
| ADC-C4 | 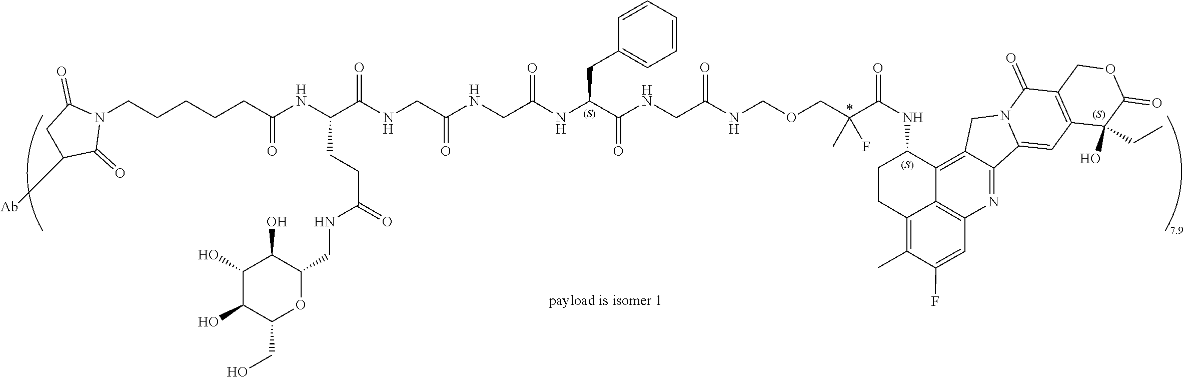 |
| ADC-C5 | 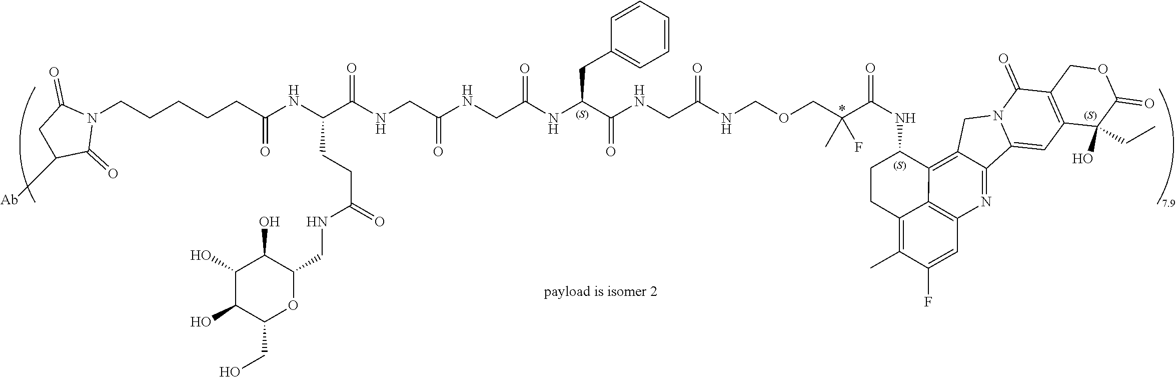 |

wherein Ab is any of the anti-B7H1-3 antibodies disclosed herein.

PC T Application No. PCT/CN2022/123665, the entire contents of which are incorporated herein by reference, discloses antibody drug conjugates, the linker payload portions of which are suitable for use in the context of the present disclosure, and linker payloads which are suitable for use in the context of the present disclosure. In some embodiments, a linker payload is a linker payload disclosed in PCT/CN2022/123665.

In some embodiments (e.g., of a compound of Formula XI), PA is a residue of:

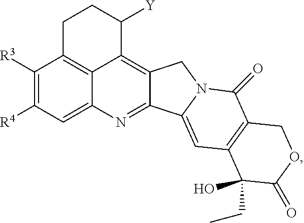

wherein
Y is -A-B—C′-D′-H;
A is a bond, CR¹R², or N—R¹;
B is a bond, —C(═O)—; or —C(═O)O—;
C is a bond, or a divalent group, wherein the divalent group is unsubstituted or substituted C₁₋₈alkyl, unsubstituted or substituted cycloalkyl, unsubstituted or substituted heterocyclyl, unsubstituted or substituted aryl, or unsubstituted or substituted heteroaryl;
D′ is a bond, NH, or O;
each of R¹, and R² is, independently, hydrogen, halogen, substituted or unsubstituted alkyl, or substituted or unsubstituted alkoxyl; or R¹, and R² together with the atom to which they are attached form unsubstituted or substituted cycloalkyl, unsubstituted or substituted heterocyclyl, unsubstituted or substituted aryl, or unsubstituted or substituted heteroaryl; and
each of R³, and R⁴ is, independently, hydrogen, halogen, substituted or unsubstituted alkyl, or substituted or unsubstituted alkoxyl; or R³ and R⁴ together with the atoms to which they are attached form unsubstituted or substituted cycloalkyl, unsubstituted or substituted heterocyclyl, unsubstituted or substituted aryl, or unsubstituted or substituted heteroaryl. In some embodiments, when R³ is methyl, and R⁴ is F, Y is not —NH—C(═O)—C-D-H.

In embodiments, in the residue of the payload depicted above, Y is -A-B-C′-D′-, the result of removal of —H from -A-B-C′-D′-H. It will be appreciated that while a payload residue can result from removal of hydrogen atom from a payload depicted herein, it also can result from removal of a hydroxy group, such as a hydroxy group formed when D′ is O in the payload depicted above (or a corresponding hydroxy group in any of the other payload structures depicted herein).

In some embodiments, the PA is a residue of:

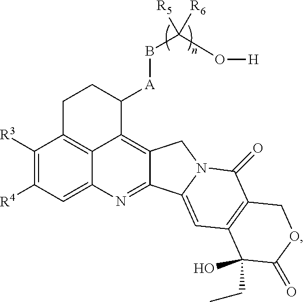

wherein
A is CR¹R², NH, or N—R¹;
B is a bond, —C(═O)—; or —C(═O)O—;
each of R¹, and R² is, independently, H, or C₁₋₄alkyl;
each of R³, and R⁴ is, independently, hydrogen, halogen, substituted or unsubstituted alkyl, or substituted or unsubstituted alkoxyl; or R³ and R⁴ together with the atoms to which they are attached form unsubstituted or substituted cycloalkyl, unsubstituted or substituted heterocyclyl, unsubstituted or substituted aryl, or unsubstituted or substituted heteroaryl;
each of R⁵, and R⁶ is, independently, hydrogen, halogen, substituted or unsubstituted alkyl, or substituted or unsubstituted alkoxyl; and
n is 1, 2, 3, 4, or 5.

In some embodiments, A is —CH₂—, and B is a bond.

In some embodiments, R⁵ and R⁶ are hydrogen, and n is 1, 2, or 3.

In some embodiments, R³ is methyl, and R⁴ is F.

In some embodiments, PA is a residue of:

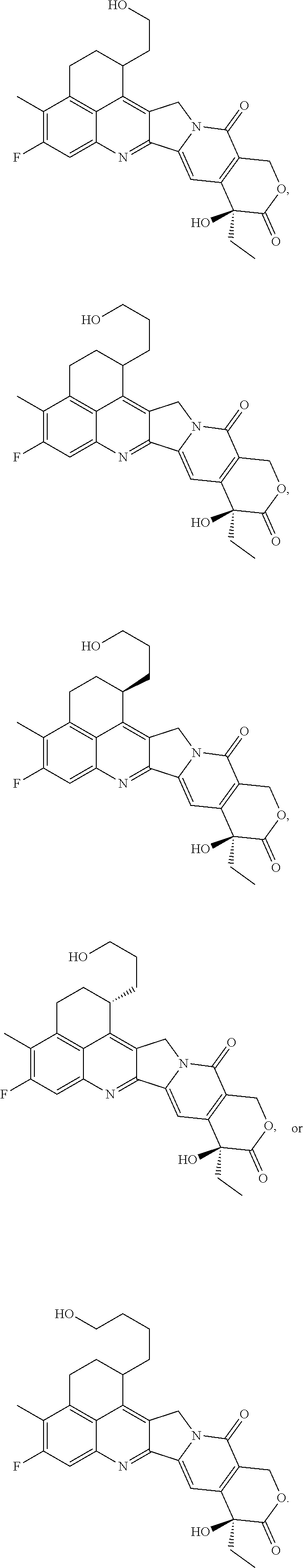 [SMILES: CC[C@@]1(O)C(=O)OCc2c1cc1n(c2=O)Cc2c-1nc1cc(F)c(C)c3c1c2C(CCO)CC3 ; CC[C@@]1(O)C(=O)OCc2c1cc1n(c2=O)Cc2c-1nc1cc(F)c(C)c3c1c2C(CCCO)CC3 ; CC[C@@]1(O)C(=O)OCc2c1cc1n(c2=O)Cc2c-1nc1cc(F)c(C)c3c1c2[C@H](CCCO)CC3 ; CC[C@@]1(O)C(=O)OCc2c1cc1n(c2=O)Cc2c-1nc1cc(F)c(C)c3c1c2[C@@H](CCCO)CC3 ; CC[C@@]1(O)C(=O)OCc2c1cc1n(c2=O)Cc2c-1nc1cc(F)c(C)c3c1c2C(CCCCO)CC3]

In some embodiments, R³, and R⁴ together with the atoms to which they are attached, form an unsubstituted or substituted dioxole ring.

In some embodiments, PA is a residue of:

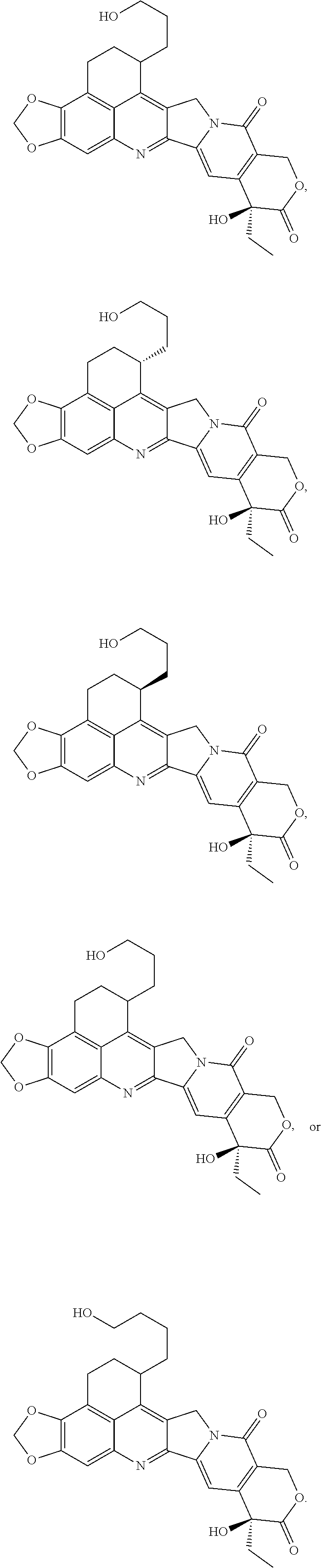 [SMILES: CC[C@@]1(O)C(=O)OCc2c1cc1n(c2=O)Cc2c-1nc1cc3c(c4c1c2C(CCCO)CC4)OCO3 ; CC[C@@]1(O)C(=O)OCc2c1cc1n(c2=O)Cc2c-1nc1cc3c(c4c1c2[C@@H](CCCO)CC4)OCO3 ; CC[C@@]1(O)C(=O)OCc2c1cc1n(c2=O)Cc2c-1nc1cc3c(c4c1c2[C@H](CCCO)CC4)OCO3 ; CC[C@@]1(O)C(=O)OCc2c1cc1n(c2=O)Cc2c-1nc1cc3c(c4c1c2C(CCCO)CC4)OCO3 ; CC[C@@]1(O)C(=O)OCc2c1cc1n(c2=O)Cc2c-1nc1cc3c(c4c1c2C(CCCCO)CC4)OCO3]

In some embodiments, A is —N(CH₃)—, and B is a bond.

In some embodiments, R⁵ and R⁶ are hydrogen, and n is 2.

In some embodiments, PA is a residue of:

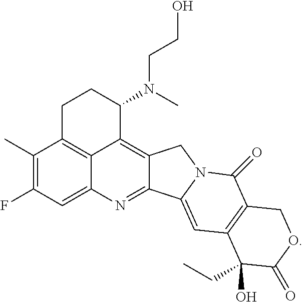 [SMILES: CC[C@@]1(O)C(=O)OCc2c1cc1n(c2=O)Cc2c-1nc1cc(F)c(C)c3c1c2[C@@H](N(C)CCO)CC3]

In some embodiments, A is —NH—, and B is —C(═O)O—. In further embodiments, R⁵ and R⁶ are hydrogen, and n is 2.

In some embodiments, PA is a residue of:

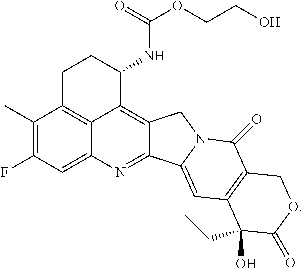 [SMILES: CC[C@@]1(O)C(=O)OCc2c1cc1n(c2=O)Cc2c-1nc1cc(F)c(C)c3c1c2[C@@H](NC(=O)OCCO)CC3]

In some embodiments, A is —NH—, and B is —C(═O)—. In further embodiments, R⁵ and R⁶ are hydrogen, and n is 2.

In some embodiments, PA is a residue of

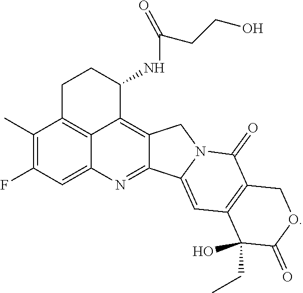 [SMILES: CC[C@@]1(O)C(=O)OCc2c1cc1n(c2=O)Cc2c-1nc1cc(F)c(C)c3c1c2[C@@H](NC(=O)CCO)CC3]

In some embodiments, R³ is Cl, R⁴ is F, and B is —C(═O)—.

In some embodiments, PA is a residue of

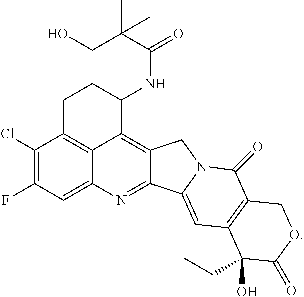 [SMILES: CC[C@@]1(O)C(=O)OCc2c1cc1n(c2=O)Cc2c-1nc1cc(F)c(Cl)c3c1c2C(NC(=O)C(C)(C)CO)CC3]

In some embodiments, R is methyl, R⁴ is Cl, and B is —C(═O)—.

In some embodiments, PA is a residue of

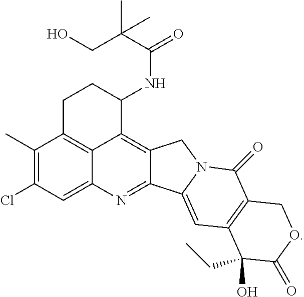 [SMILES: CC[C@@]1(O)C(=O)OCc2c1cc1n(c2=O)Cc2c-1nc1cc(Cl)c(C)c3c1c2C(NC(=O)C(C)(C)CO)CC3]

In some embodiments, R³, and R⁴ together with the atoms to which they are attached form unsubstituted or substituted heterocyclyl.

In some embodiments, R³, and R⁴ together with the atoms to which they are attached form an unsubstituted or substituted dioxole ring, and B is —C(═O)—.

In some embodiments, PA is a residue of:

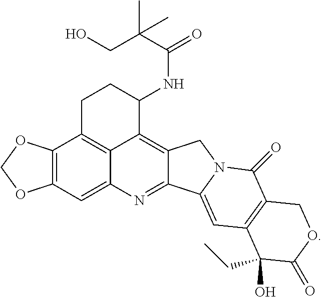 [SMILES: CC[C@@]1(O)C(=O)OCc2c1cc1n(c2=O)Cc2c-1nc1cc3c(c4c1c2C(NC(=O)C(C)(C)CO)CC4)OCO3]

In some embodiments, PA is a residue of:

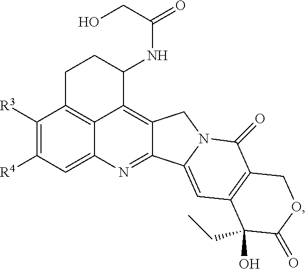

wherein values and alternative values for the variables (e.g., R³, R⁴) are as described elsewhere herein.

In some embodiments, R is methyl; and R⁴ is Cl.

In some embodiments, PA is a residue of:

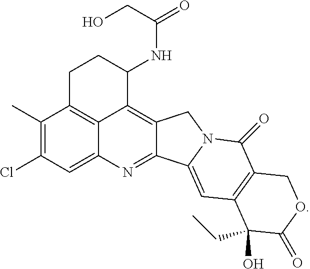 [SMILES: CC[C@@]1(O)C(=O)OCc2c1cc1n(c2=O)Cc2c-1nc1cc(Cl)c(C)c3c1c2C(NC(=O)CO)CC3]

In some embodiments, R³ is Cl; and R⁴ is F.

In some embodiments, PA is a residue of:

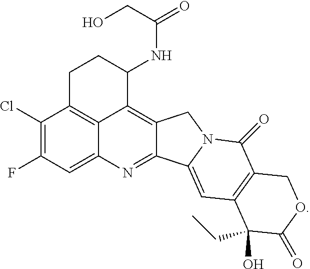 [SMILES: CC[C@@]1(O)C(=O)OCc2c1cc1n(c2=O)Cc2c-1nc1cc(F)c(Cl)c3c1c2C(NC(=O)CO)CC3]

In some embodiments, R³ is F; and R⁴ is F.

In some embodiments, PA is a residue of:

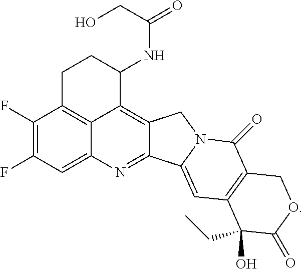 [SMILES: CC[C@@]1(O)C(=O)OCc2c1cc1n(c2=O)Cc2c-1nc1cc(F)c(F)c3c1c2C(NC(=O)CO)CC3]

In some embodiments, R³ is H; and R⁴ is F.

In some embodiments, PA is a residue of

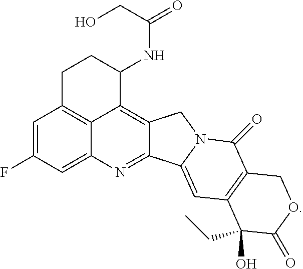 [SMILES: CC[C@@]1(O)C(=O)OCc2c1cc1n(c2=O)Cc2c-1nc1cc(F)cc3c1c2C(NC(=O)CO)CC3]

In some embodiments, R³ is H; and R⁴ is OH.

In some embodiments, PA is a residue of

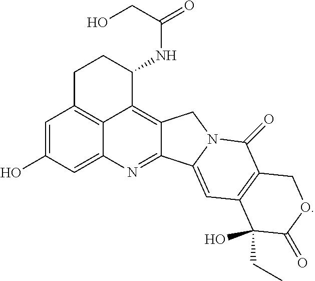 [SMILES: CC[C@@]1(O)C(=O)OCc2c1cc1n(c2=O)Cc2c-1nc1cc(O)cc3c1c2[C@@H](NC(=O)CO)CC3]

In some embodiments, R³ is methyl; and R⁴ is methyl.

In some embodiments, PA is a residue of:

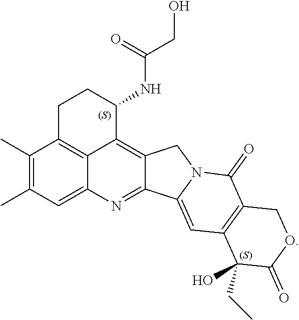

In some embodiments, R³ is methoxyl; and R⁴ is F.

In some embodiments, PA is a residue of:

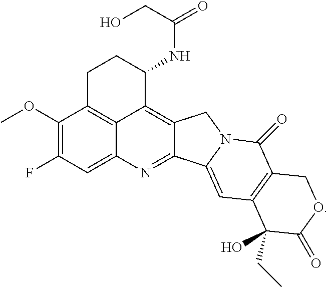 [SMILES: CC[C@@]1(O)C(=O)OCc2c1cc1n(c2=O)Cc2c-1nc1cc(F)c(OC)c3c1c2[C@@H](NC(=O)CO)CC3]

In some embodiments, R³ is H; and R⁴ is methoxyl.

In some embodiments, PA is a residue of:

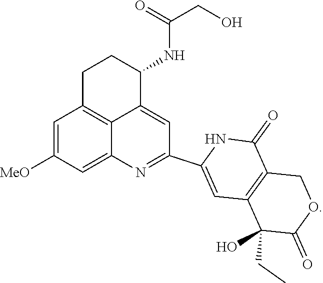 [SMILES: CC[C@@]1(O)C(=O)OCc2c1cc(-c1cc3c4c(cc(OC)cc4n1)CC[C@@H]3NC(=O)CO)[nH]c2=O]

In some embodiments, R³ is H; and R⁴ is Cl.

In some embodiments, PA is a residue of:

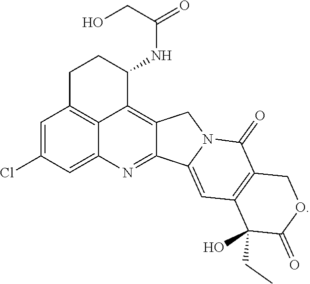 [SMILES: CC[C@@]1(O)C(=O)OCc2c1cc1n(c2=O)Cc2c-1nc1cc(Cl)cc3c1c2[C@@H](NC(=O)CO)CC3]

In some embodiments, R³ and R⁴ together with the atoms to which they are attached form unsubstituted or substituted heterocyclyl.

In some embodiments, R³ and R^{4′} together with the atoms to which they are attached form an unsubstituted or substituted dioxole ring.

In some embodiments, PA is a residue of:

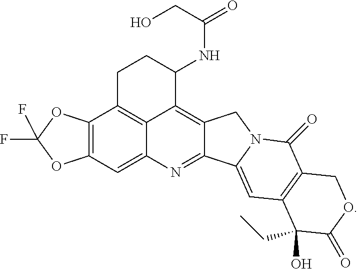 [SMILES: CC[C@@]1(O)C(=O)OCc2c1cc1n(c2=O)Cc2c-1nc1cc3c(c4c1c2C(NC(=O)CO)CC4)OC(F)(F)O3]

In some embodiments, PA is a residue of:

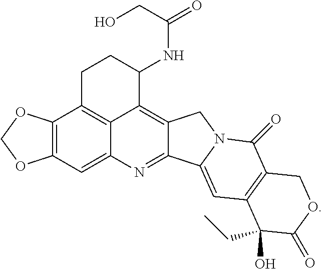 [SMILES: CC[C@@]1(O)C(=O)OCc2c1cc1n(c2=O)Cc2c-1nc1cc3c(c4c1c2C(NC(=O)CO)CC4)OCO3]

In some embodiments, PA is a residue of:

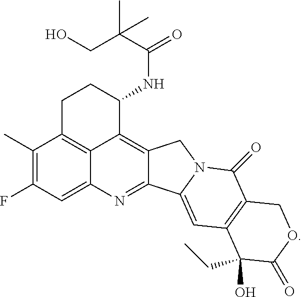 [SMILES: CC[C@@]1(O)C(=O)OCc2c1cc1n(c2=O)Cc2c-1nc1cc(F)c(C)c3c1c2[C@@H](NC(=O)C(C)(C)CO)CC3]

In some embodiments, PA is a residue of:

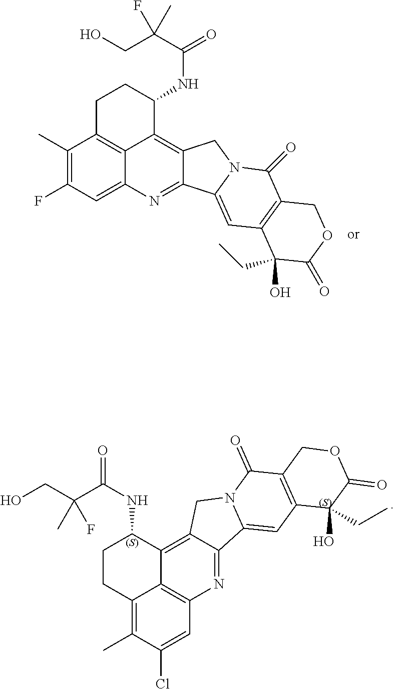

In some embodiments, PA is a residue of:

| Examples | Payload structure |
|---|---|
| 1-A | 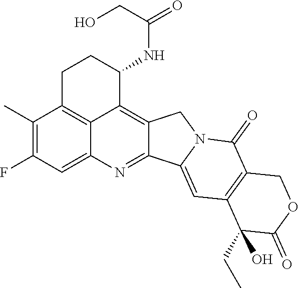 [SMILES: CC[C@@]1(O)C(=O)OCc2c1cc1n(c2=O)Cc2c-1nc1cc(F)c(C)c3c1c2[C@@H](NC(=O)CO)CC3] |
| 1-B | 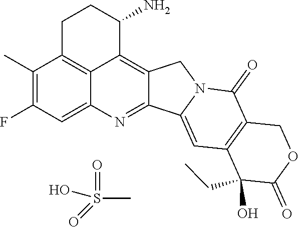 |
| 1-C | 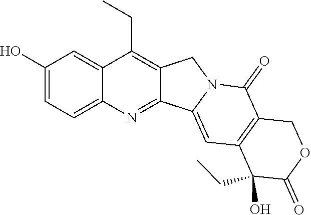 [SMILES: CCc1c2c(nc3ccc(O)cc13)-c1cc3c(c(=O)n1C2)COC(=O)[C@]3(O)CC] |
| 1-1 | 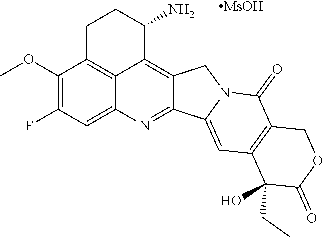 [SMILES: "·MsOH" ; CC[C@@]1(O)C(=O)OCc2c1cc1n(c2=O)Cc2c-1nc1cc(F)c(OC)c3c1c2[C@@H](N)CC3] |
| 1-2 | 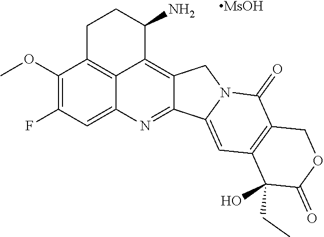 [SMILES: "·MsOH" ; CC[C@@]1(O)C(=O)OCc2c1cc1n(c2=O)Cc2c-1nc1cc(F)c(OC)c3c1c2[C@H](N)CC3] |
| 1-3 | 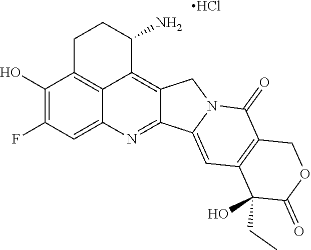 [SMILES: Cl ; CC[C@@]1(O)C(=O)OCc2c1cc1n(c2=O)Cc2c-1nc1cc(F)c(O)c3c1c2[C@@H](N)CC3] |
| 1-4 | 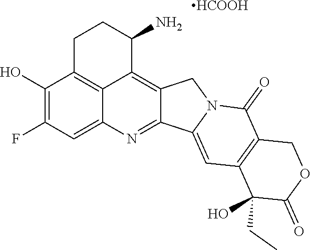 [SMILES: "·HCOOH" ; CC[C@@]1(O)C(=O)OCc2c1cc1n(c2=O)Cc2c-1nc1cc(F)c(O)c3c1c2[C@H](N)CC3] |
| 1-5 | 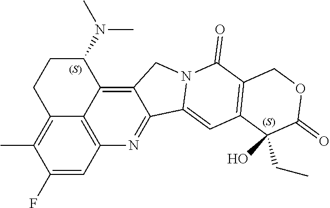 |
| 1-6 | 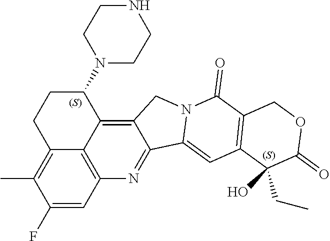 |
| 1-7 | 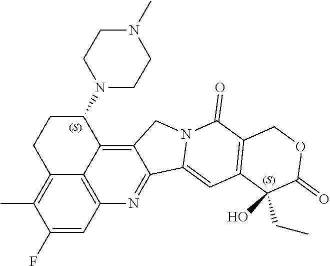 |
| 1-8 | 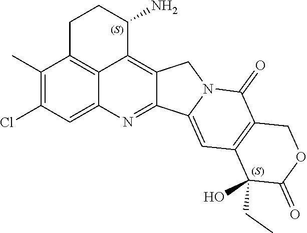 |
| 1-9 | 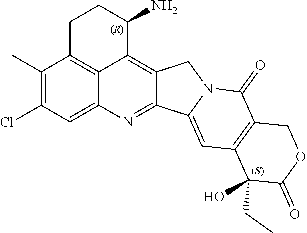 |
| 1-10 | 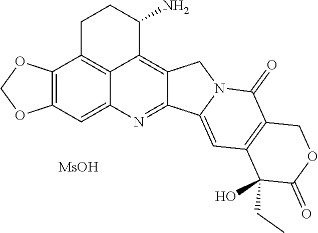 [SMILES: "MsOH" ; CC[C@@]1(O)C(=O)OCc2c1cc1n(c2=O)Cc2c-1nc1cc3c(c4c1c2[C@@H](N)CC4)OCO3] |
| 1-11 | 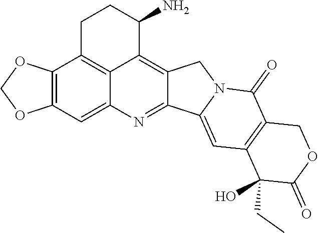 [SMILES: CC[C@@]1(O)C(=O)OCc2c1cc1n(c2=O)Cc2c-1nc1cc3c(c4c1c2[C@H](N)CC4)OCO3] |
| 1-12 | 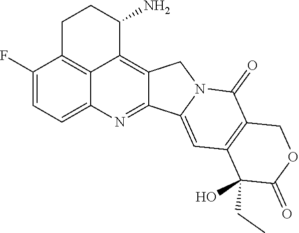 [SMILES: CC[C@@]1(O)C(=O)OCc2c1cc1n(c2=O)Cc2c-1nc1ccc(F)c3c1c2[C@@H](N)CC3] |
| 1-13 | 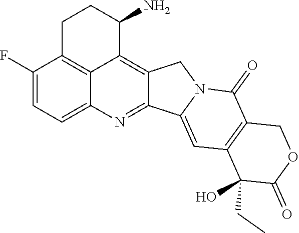 [SMILES: CC[C@@]1(O)C(=O)OCc2c1cc1n(c2=O)Cc2c-1nc1ccc(F)c3c1c2[C@H](N)CC3] |
| 1-14 | 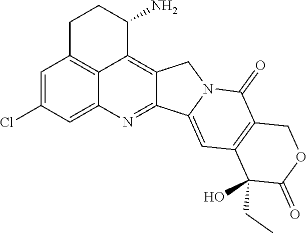 [SMILES: CC[C@@]1(O)C(=O)OCc2c1cc1n(c2=O)Cc2c-1nc1cc(Cl)cc3c1c2[C@@H](N)CC3] |
| 1-15 | 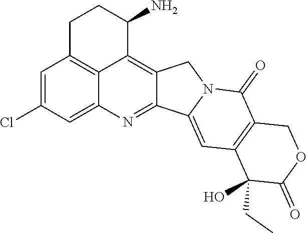 [SMILES: CC[C@@]1(O)C(=O)OCc2c1cc1n(c2=O)Cc2c-1nc1cc(Cl)cc3c1c2[C@H](N)CC3] |
| 1-16 | 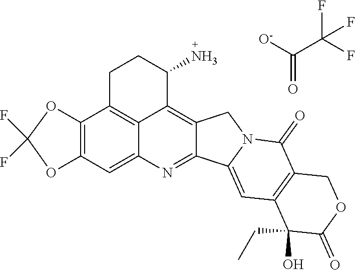 [SMILES: CC[C@@]1(O)C(=O)OCc2c1cc1n(c2=O)Cc2c-1nc1cc3c(c4c1c2[C@@H](N)CC4)OC(F)(F)O3 ; O=C([O-])C(F)(F)F] |
| 1-17 | 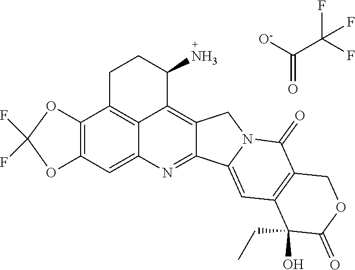 [SMILES: CC[C@@]1(O)C(=O)OCc2c1cc1n(c2=O)Cc2c-1nc1cc3c(c4c1c2[C@H](N)CC4)OC(F)(F)O3 ; O=C([O-])C(F)(F)F] |
| 1-18 | 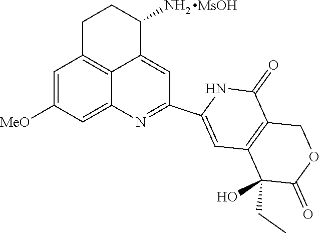 [SMILES: "·MsOH" ; CC[C@@]1(O)C(=O)OCc2c1cc(-c1cc3c4c(cc(OC)cc4n1)CC[C@@H]3N)[nH]c2=O] |
| 1-19 | 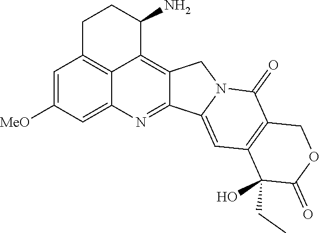 [SMILES: CC[C@@]1(O)C(=O)OCc2c1cc1n(c2=O)Cc2c-1nc1cc(OC)cc3c1c2[C@H](N)CC3] |
| 1-20 | 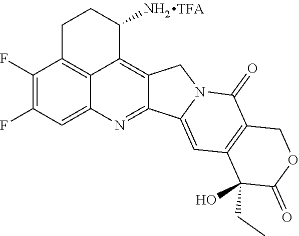 [SMILES: CC[C@@]1(O)C(=O)OCc2c1cc1n(c2=O)Cc2c-1nc1cc(F)c(F)c3c1c2[C@@H](N)CC3] |
| 1-21 | 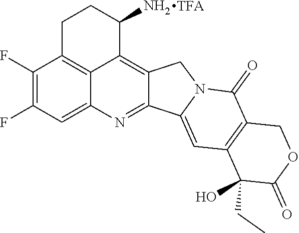 [SMILES: CC[C@@]1(O)C(=O)OCc2c1cc1n(c2=O)Cc2c-1nc1cc(F)c(F)c3c1c2[C@H](N)CC3 ; "·TFA"] |
| 1-22 | 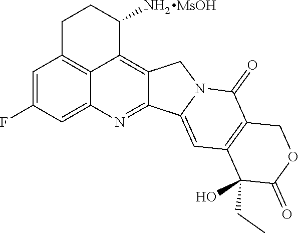 [SMILES: "·MsOH" ; CC[C@@]1(O)C(=O)OCc2c1cc1n(c2=O)Cc2c-1nc1cc(F)cc3c1c2[C@@H](N)CC3] |
| 1-23 | 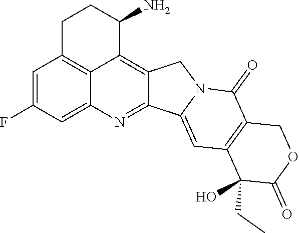 [SMILES: CC[C@@]1(O)C(=O)OCc2c1cc1n(c2=O)Cc2c-1nc1cc(F)cc3c1c2[C@H](N)CC3] |
| 1-24 | 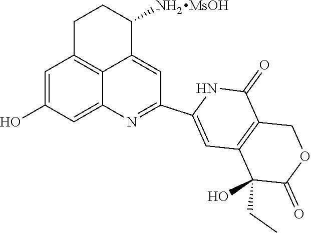 [SMILES: "·MsOH" ; CC[C@@]1(O)C(=O)OCc2c1cc(-c1cc3c4c(cc(O)cc4n1)CC[C@@H]3N)[nH]c2=O] |
| 1-25 | 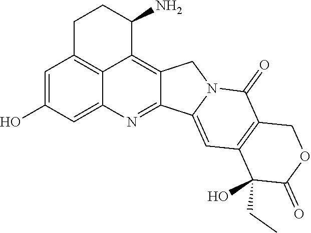 [SMILES: CC[C@@]1(O)C(=O)OCc2c1cc1n(c2=O)Cc2c-1nc1cc(O)cc3c1c2[C@H](N)CC3] |
| 1-26 | 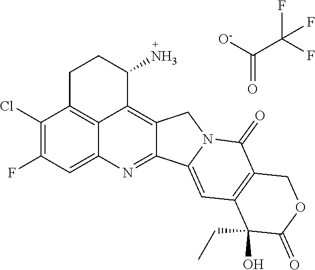 [SMILES: CC[C@@]1(O)C(=O)OCc2c1cc1n(c2=O)Cc2c-1nc1cc(F)c(Cl)c3c1c2[C@@H](N)CC3 ; O=C([O-])C(F)(F)F] |
| 1-27 | 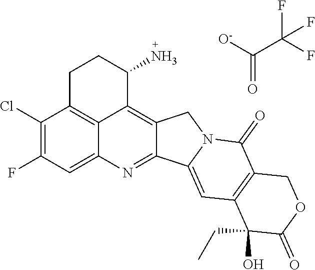 [SMILES: CC[C@@]1(O)C(=O)OCc2c1cc1n(c2=O)Cc2c-1nc1cc(F)c(Cl)c3c1c2[C@@H](N)CC3 ; O=C([O-])C(F)(F)F] |

In some embodiments, PA is a residue of:

| Compound | |
|---|---|
| number | Payload structure |
| 2-1 | 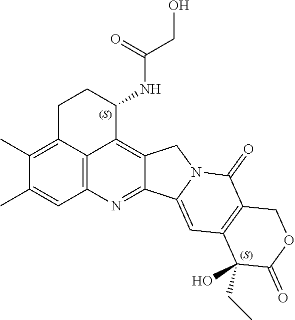 |
| 2-2 | 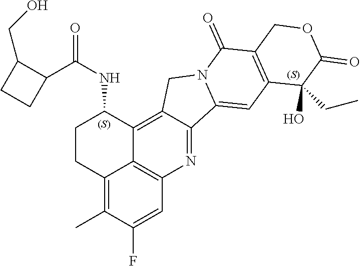 |
| 2-3 | 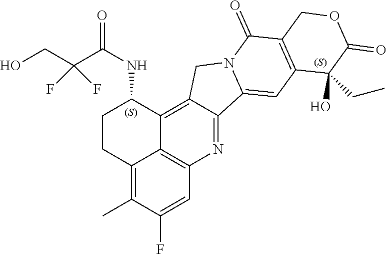 |
| 2-4 | 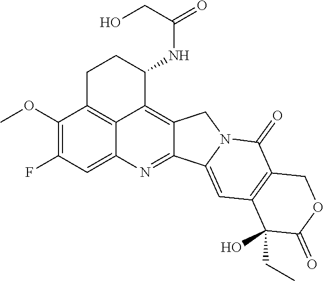 [SMILES: CC[C@@]1(O)C(=O)OCc2c1cc1n(c2=O)Cc2c-1nc1cc(F)c(OC)c3c1c2[C@@H](NC(=O)CO)CC3] |
| 2-5 | 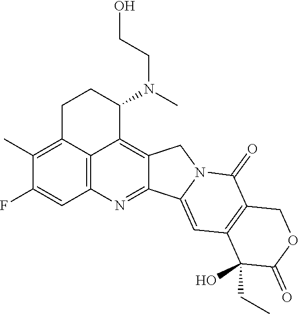 [SMILES: CC[C@@]1(O)C(=O)OCc2c1cc1n(c2=O)Cc2c-1nc1cc(F)c(C)c3c1c2[C@@H](N(C)CCO)CC3] |
| 2-6 | 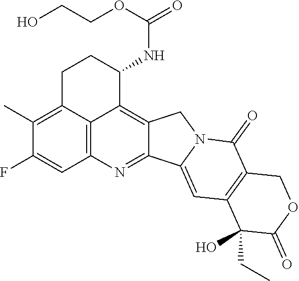 [SMILES: CC[C@@]1(O)C(=O)OCc2c1cc1n(c2=O)Cc2c-1nc1cc(F)c(C)c3c1c2[C@@H](NC(=O)OCCO)CC3] |
| 2-7 | 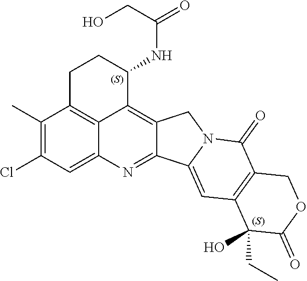 |
| 2-8 | 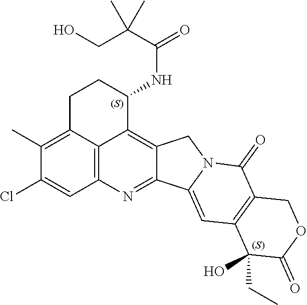 |
| 2-9 | 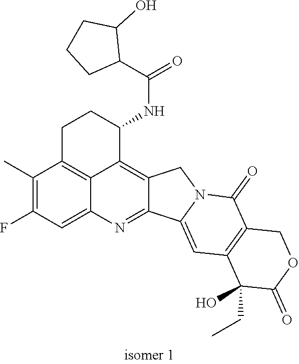 [SMILES: CC[C@@]1(O)C(=O)OCc2c1cc1n(c2=O)Cc2c-1nc1cc(F)c(C)c3c1c2[C@@H](NC(=O)C1CCCC1O)CC3] |
| 2-10 | 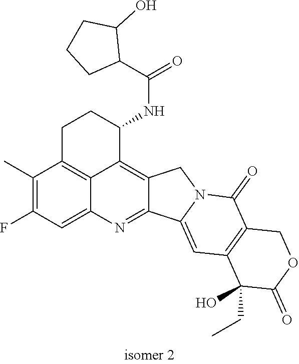 [SMILES: CC[C@@]1(O)C(=O)OCc2c1cc1n(c2=O)Cc2c-1nc1cc(F)c(C)c3c1c2[C@@H](NC(=O)C1CCCC1O)CC3] |
| 2-11 | 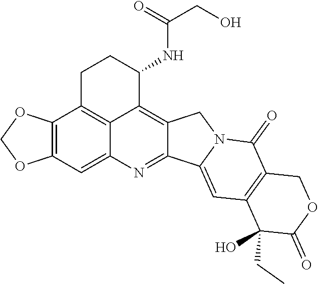 [SMILES: CC[C@@]1(O)C(=O)OCc2c1cc1n(c2=O)Cc2c-1nc1cc3c(c4c1c2[C@@H](NC(=O)CO)CC4)OCO3] |
| 2-12 | 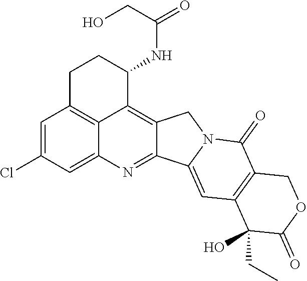 [SMILES: CC[C@@]1(O)C(=O)OCc2c1cc1n(c2=O)Cc2c-1nc1cc(Cl)cc3c1c2[C@@H](NC(=O)CO)CC3] |
| 2-13 | 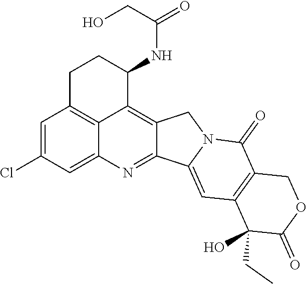 [SMILES: CC[C@@]1(O)C(=O)OCc2c1cc1n(c2=O)Cc2c-1nc1cc(Cl)cc3c1c2[C@H](NC(=O)CO)CC3] |
| 2-14 | 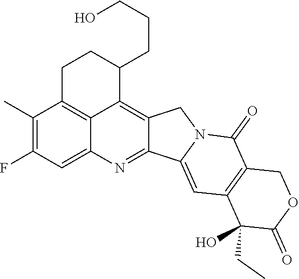 [SMILES: CC[C@@]1(O)C(=O)OCc2c1cc1n(c2=O)Cc2c-1nc1cc(F)c(C)c3c1c2C(CCCO)CC3] |
| 2-15 | 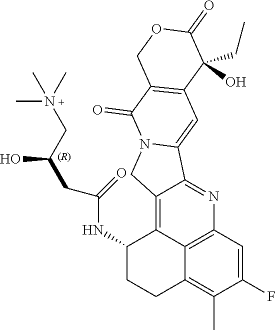 |
| 2-16 | 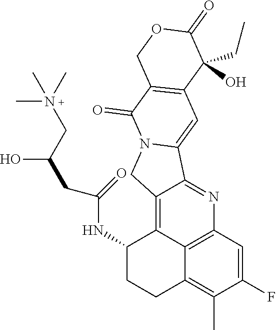 |
| 2-17 | 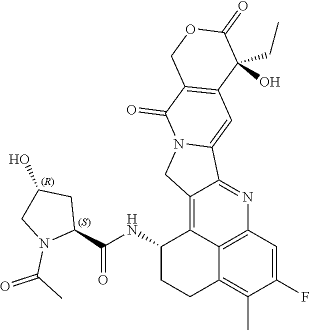 |
| 2-18 | 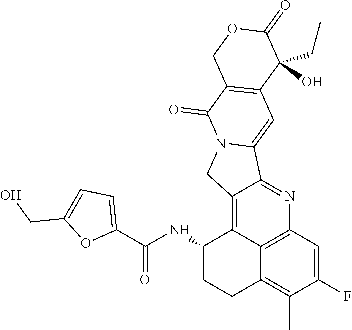 [SMILES: CC[C@@]1(O)C(=O)OCc2c1cc1n(c2=O)Cc2c-1nc1cc(F)c(C)c3c1c2[C@@H](NC(=O)c1ccc(CO)o1)CC3] |
| 2-19 | 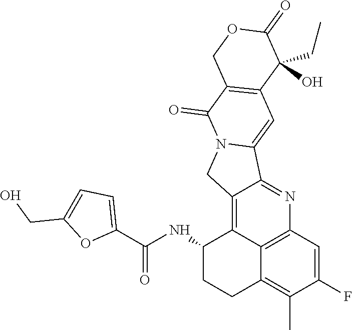 [SMILES: CC[C@@]1(O)C(=O)OCc2c1cc1n(c2=O)Cc2c-1nc1cc(F)c(C)c3c1c2[C@@H](NC(=O)c1ccc(CO)o1)CC3] |
| 2-20 | 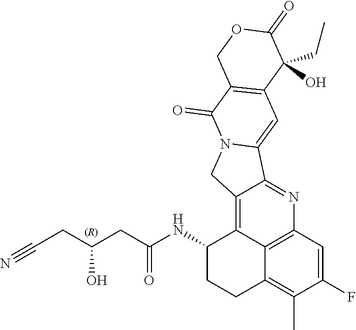 |
| 2-21 | 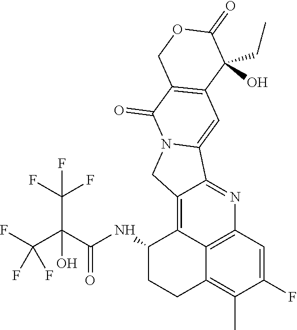 [SMILES: CC[C@@]1(O)C(=O)OCc2c1cc1n(c2=O)Cc2c-1nc1cc(F)c(C)c3c1c2[C@@H](NC(=O)C(O)(C(F)(F)F)C(F)(F)F)CC3] |
| 2-22 | 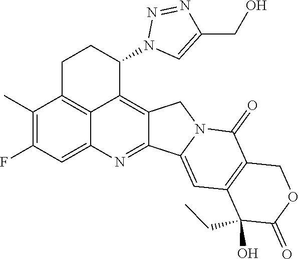 [SMILES: CC[C@@]1(O)C(=O)OCc2c1cc1n(c2=O)Cc2c-1nc1cc(F)c(C)c3c1c2[C@@H](n1cc(CO)nn1)CC3] |
| 2-23 | 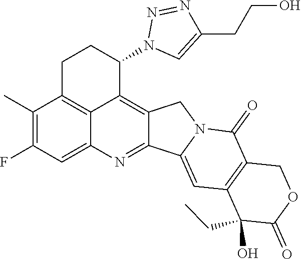 [SMILES: CC[C@@]1(O)C(=O)OCc2c1cc1n(c2=O)Cc2c-1nc1cc(F)c(C)c3c1c2[C@@H](n1cc(CCO)nn1)CC3] |
| 2-24 | 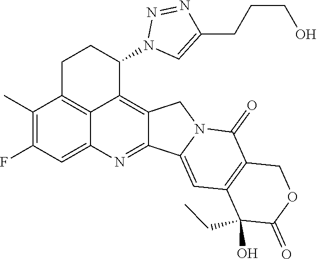 [SMILES: CC[C@@]1(O)C(=O)OCc2c1cc1n(c2=O)Cc2c-1nc1cc(F)c(C)c3c1c2[C@@H](n1cc(CCCO)nn1)CC3] |
| 2-25 | 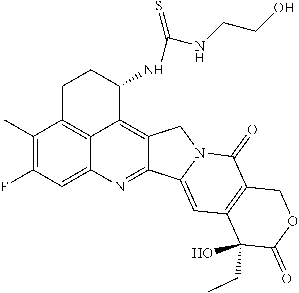 [SMILES: CC[C@@]1(O)C(=O)OCc2c1cc1n(c2=O)Cc2c-1nc1cc(F)c(C)c3c1c2[C@@H](NC(=S)NCCO)CC3] |
| 2-26 | 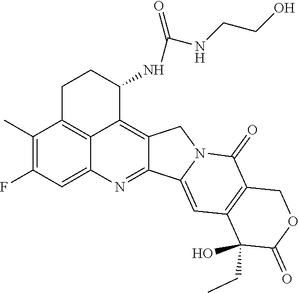 [SMILES: CC[C@@]1(O)C(=O)OCc2c1cc1n(c2=O)Cc2c-1nc1cc(F)c(C)c3c1c2[C@@H](NC(=O)NCCO)CC3] |
| 2-27 | 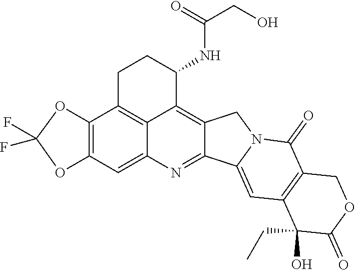 [SMILES: CC[C@@]1(O)C(=O)OCc2c1cc1n(c2=O)Cc2c-1nc1cc3c(c4c1c2[C@@H](NC(=O)CO)CC4)OC(F)(F)O3] |
| 2-28 | 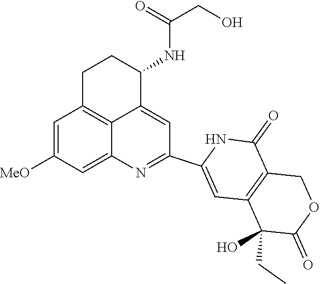 [SMILES: CC[C@@]1(O)C(=O)OCc2c1cc(-c1cc3c4c(cc(OC)cc4n1)CC[C@@H]3NC(=O)CO)[nH]c2=O] |
| 2-29 | 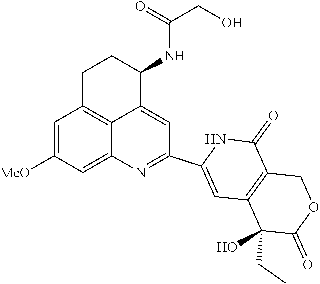 [SMILES: CC[C@@]1(O)C(=O)OCc2c1cc(-c1cc3c4c(cc(OC)cc4n1)CC[C@H]3NC(=O)CO)[nH]c2=O] |
| 2-30 | 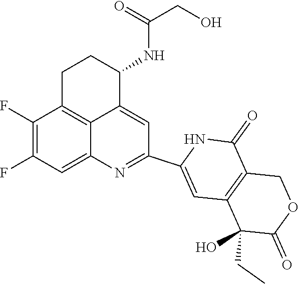 [SMILES: CC[C@@]1(O)C(=O)OCc2c1cc(-c1cc3c4c(c(F)c(F)cc4n1)CC[C@@H]3NC(=O)CO)[nH]c2=O] |
| 2-31 | 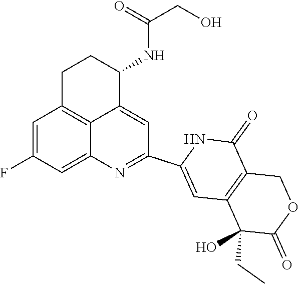 [SMILES: CC[C@@]1(O)C(=O)OCc2c1cc(-c1cc3c4c(cc(F)cc4n1)CC[C@@H]3NC(=O)CO)[nH]c2=O] |
| 2-32 | 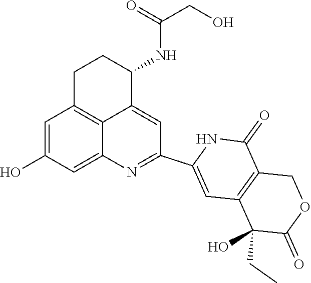 [SMILES: CC[C@@]1(O)C(=O)OCc2c1cc(-c1cc3c4c(cc(O)cc4n1)CC[C@@H]3NC(=O)CO)[nH]c2=O] |
| 2-34 | 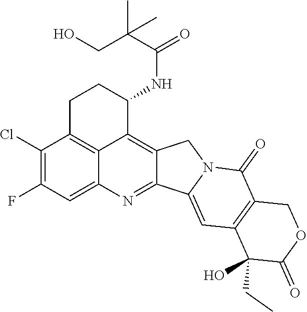 [SMILES: CC[C@@]1(O)C(=O)OCc2c1cc1n(c2=O)Cc2c-1nc1cc(F)c(Cl)c3c1c2[C@@H](NC(=O)C(C)(C)CO)CC3] |
| 2-35 | 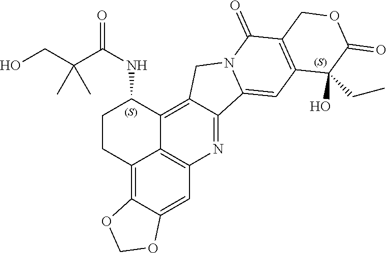 |
| 2-36 | 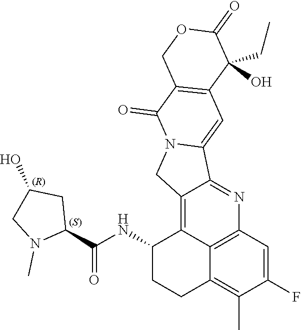 |
| 2-37 | 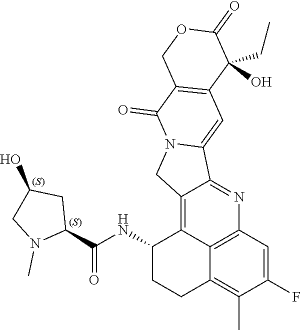 |
| 2-38 | 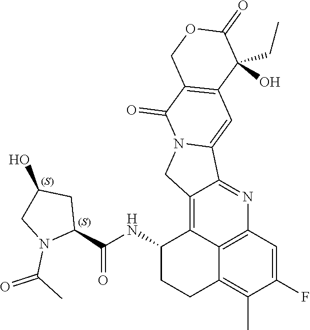 |
| 2-39 | 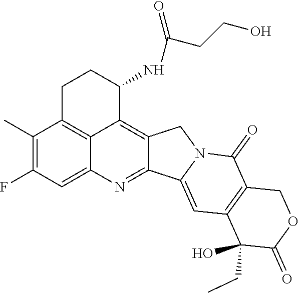 [SMILES: CC[C@@]1(O)C(=O)OCc2c1cc1n(c2=O)Cc2c-1nc1cc(F)c(C)c3c1c2[C@@H](NC(=O)CCO)CC3] |
| 2-40 | 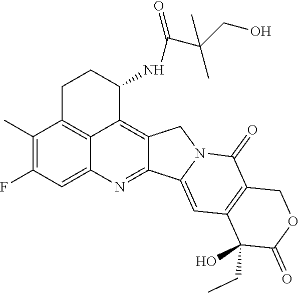 [SMILES: CC[C@@]1(O)C(=O)OCc2c1cc1n(c2=O)Cc2c-1nc1cc(F)c(C)c3c1c2[C@@H](NC(=O)C(C)(C)CO)CC3] |
| 2-41 | 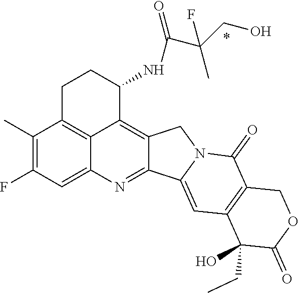 [SMILES: CC[C@@]1(O)C(=O)OCc2c1cc1n(c2=O)Cc2c-1nc1cc(F)c(C)c3c1c2[C@@H](NC(=O)C(C)(F)CO)CC3] |
| 2-42 | 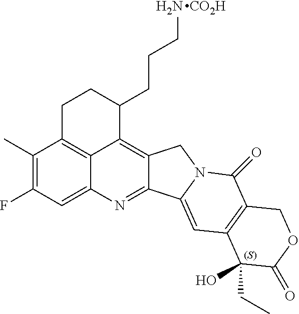 |
| 2-43 | 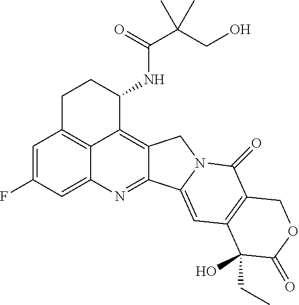 [SMILES: CC[C@@]1(O)C(=O)OCc2c1cc1n(c2=O)Cc2c-1nc1cc(F)cc3c1c2[C@@H](NC(=O)C(C)(C)CO)CC3] |
| 2-44 (isomer 1) 2-45 (isomer 2) | 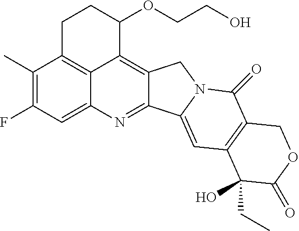 [SMILES: CC[C@@]1(O)C(=O)OCc2c1cc1n(c2=O)Cc2c-1nc1cc(F)c(C)c3c1c2C(OCCO)CC3] |
| 2-46 | 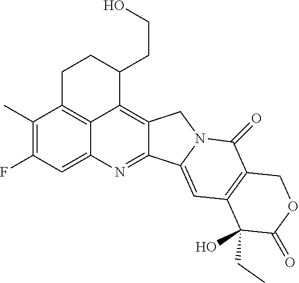 [SMILES: CC[C@@]1(O)C(=O)OCc2c1cc1n(c2=O)Cc2c-1nc1cc(F)c(C)c3c1c2C(CCO)CC3] |
| 2-47 | 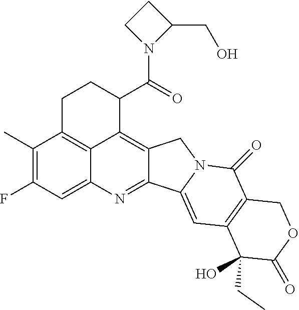 [SMILES: CC[C@@]1(O)C(=O)OCc2c1cc1n(c2=O)Cc2c-1nc1cc(F)c(C)c3c1c2C(C(=O)N1CCC1CO)CC3] |
| 2-48 | 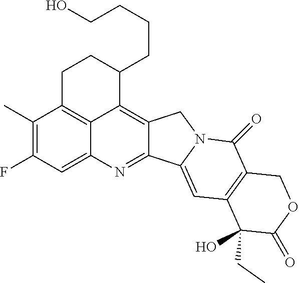 [SMILES: CC[C@@]1(O)C(=O)OCc2c1cc1n(c2=O)Cc2c-1nc1cc(F)c(C)c3c1c2C(CCCCO)CC3] |
| 2-49 (isomer 1) 2-50 (isomer 2) | 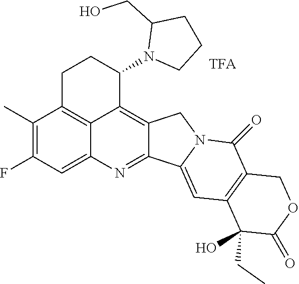 [SMILES: CC[C@@]1(O)C(=O)OCc2c1cc1n(c2=O)Cc2c-1nc1cc(F)c(C)c3c1c2[C@@H](N1CCCC1CO)CC3] |
| 2-51 (isomer 1) 2-52 (isomer 2) | 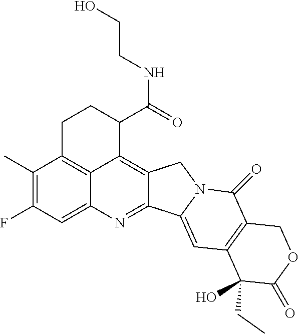 [SMILES: CC[C@@]1(O)C(=O)OCc2c1cc1n(c2=O)Cc2c-1nc1cc(F)c(C)c3c1c2C(C(=O)NCCO)CC3] |
| 2-53 | 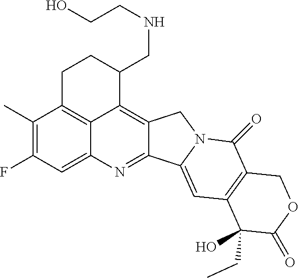 [SMILES: CC[C@@]1(O)C(=O)OCc2c1cc1n(c2=O)Cc2c-1nc1cc(F)c(C)c3c1c2C(CNCCO)CC3] |
| 2-54 | 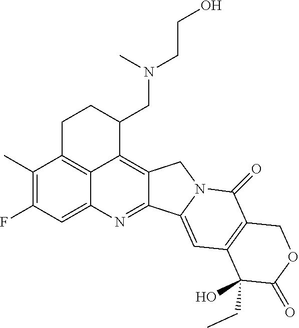 [SMILES: CC[C@@]1(O)C(=O)OCc2c1cc1n(c2=O)Cc2c-1nc1cc(F)c(C)c3c1c2C(CN(C)CCO)CC3] |
| 2-55 | 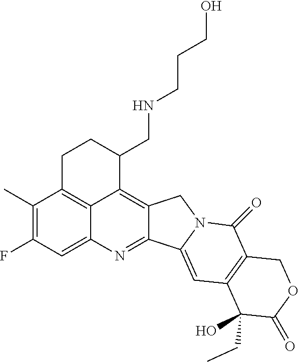 [SMILES: CC[C@@]1(O)C(=O)OCc2c1cc1n(c2=O)Cc2c-1nc1cc(F)c(C)c3c1c2C(CNCCCO)CC3] |
| 2-56 (isomer 1) 2-57 (isomer 2) | 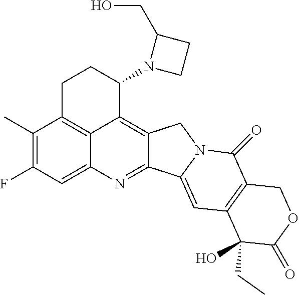 [SMILES: CC[C@@]1(O)C(=O)OCc2c1cc1n(c2=O)Cc2c-1nc1cc(F)c(C)c3c1c2[C@@H](N1CCC1CO)CC3] |
| 2-58 | 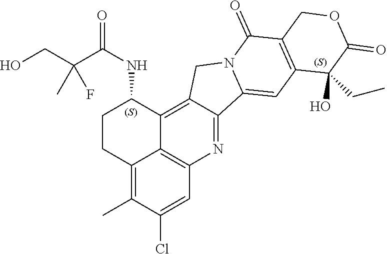 |
| 2-59 (isomer 1) 2-60 (isomer 2) | 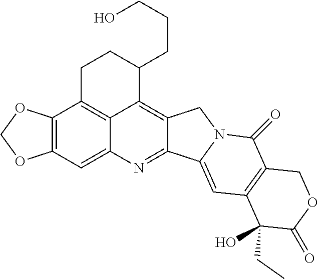 [SMILES: CC[C@@]1(O)C(=O)OCc2c1cc1n(c2=O)Cc2c-1nc1cc3c(c4c1c2C(CCCO)CC4)OCO3] |
| 2-61 | 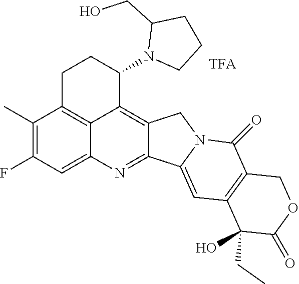 [SMILES: CC[C@@]1(O)C(=O)OCc2c1cc1n(c2=O)Cc2c-1nc1cc(F)c(C)c3c1c2[C@@H](N1CCCC1CO)CC3] |
| 2-62 | 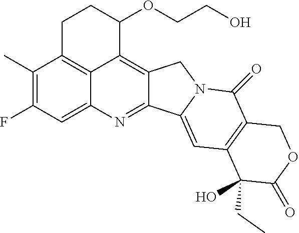 [SMILES: CC[C@@]1(O)C(=O)OCc2c1cc1n(c2=O)Cc2c-1nc1cc(F)c(C)c3c1c2C(OCCO)CC3] |
| 2-63 | 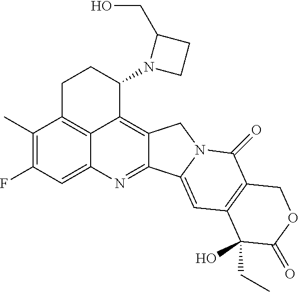 [SMILES: CC[C@@]1(O)C(=O)OCc2c1cc1n(c2=O)Cc2c-1nc1cc(F)c(C)c3c1c2[C@@H](N1CCC1CO)CC3] |
| 2-64 | 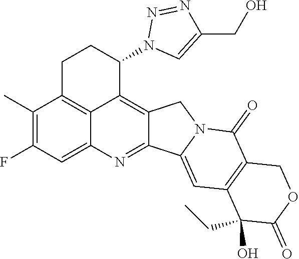 [SMILES: CC[C@@]1(O)C(=O)OCc2c1cc1n(c2=O)Cc2c-1nc1cc(F)c(C)c3c1c2[C@@H](n1cc(CO)nn1)CC3] |
| 2-65 | 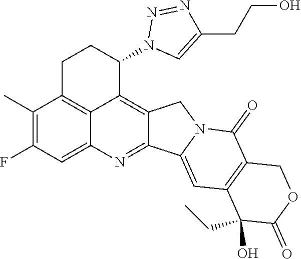 [SMILES: CC[C@@]1(O)C(=O)OCc2c1cc1n(c2=O)Cc2c-1nc1cc(F)c(C)c3c1c2[C@@H](n1cc(CCO)nn1)CC3] |

In some embodiments, PA is a residue of:

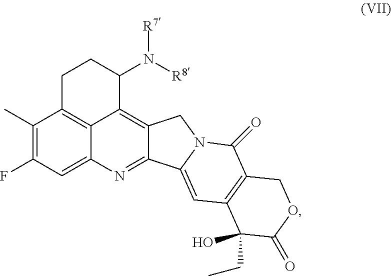

wherein
each of R^{7′} and R^{8′} is, independently, hydrogen, or substituted or unsubstituted alkyl; or R^{7′} and R^{8′} together with the nitrogen atom to which they are attached form unsubstituted or substituted heterocyclyl, or unsubstituted or substituted heteroaryl.

In some embodiments PA is a residue of:

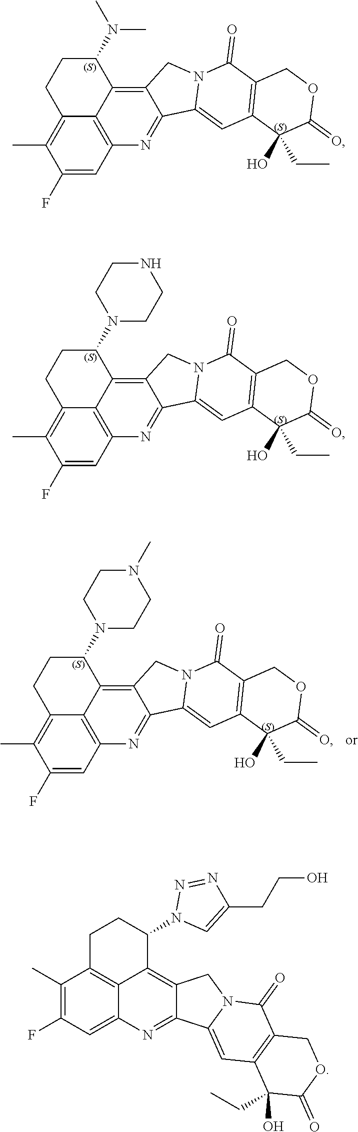

In some embodiments, an antibody drug conjugate has formula (XV):

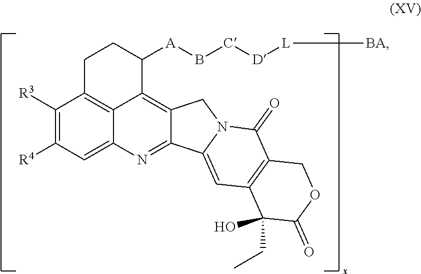

or a pharmaceutically acceptable salt, tautomer, solvate, stereoisomer, enantiomer, isotopologue, or prodrug thereof, wherein values and alternative values for the variables (e.g., A, B, C′, D′, L, R³, R⁴, and x) are as described elsewhere herein.

In some embodiments, an antibody drug conjugate has a structure of Formula (XVIIIa), (XVIIIb), or (XVIIIc):

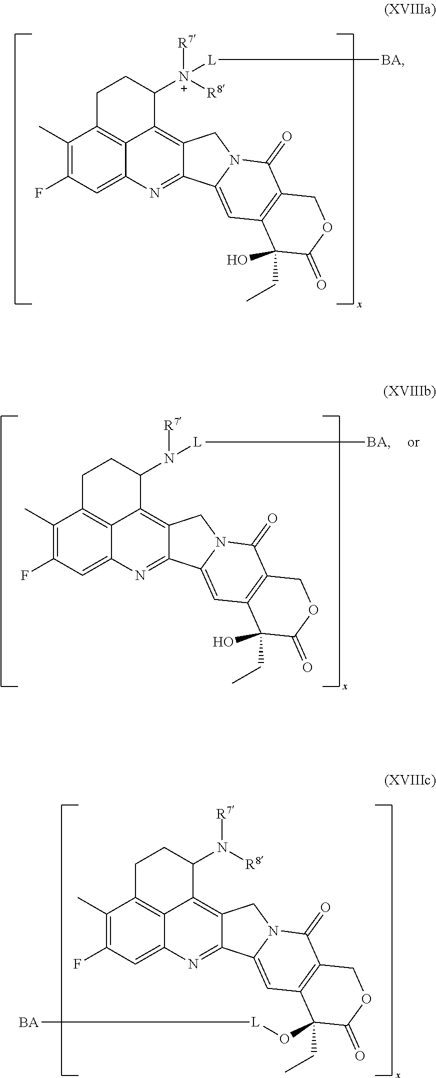

or a pharmaceutically acceptable salt, tautomer, solvate, stereoisomer, enantiomer, or isotopologue thereof, wherein values and alternative values for the variables (e.g., L, R⁷, R⁸, and x) are as described elsewhere herein.

In some embodiments, an antibody drug conjugate has a structure of any one of the following formulas:

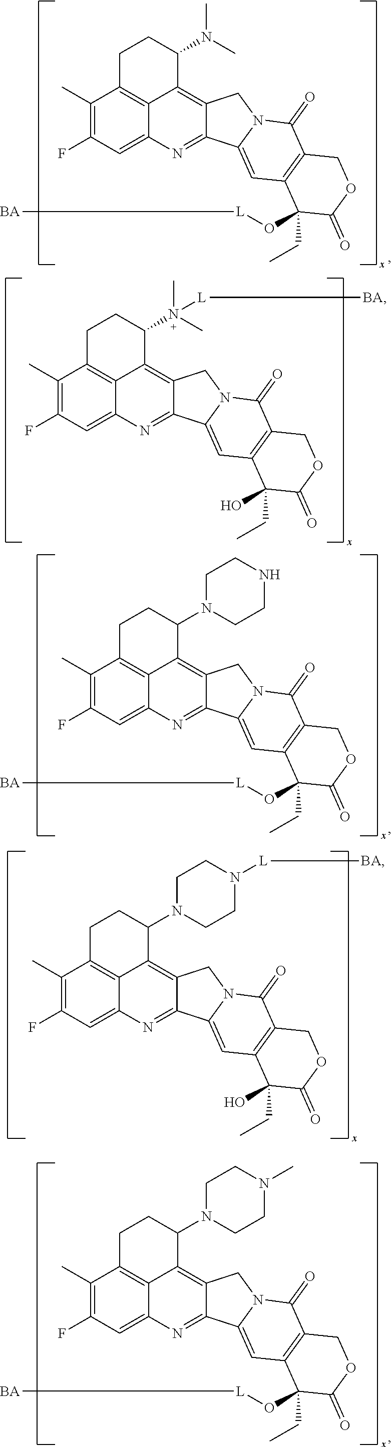

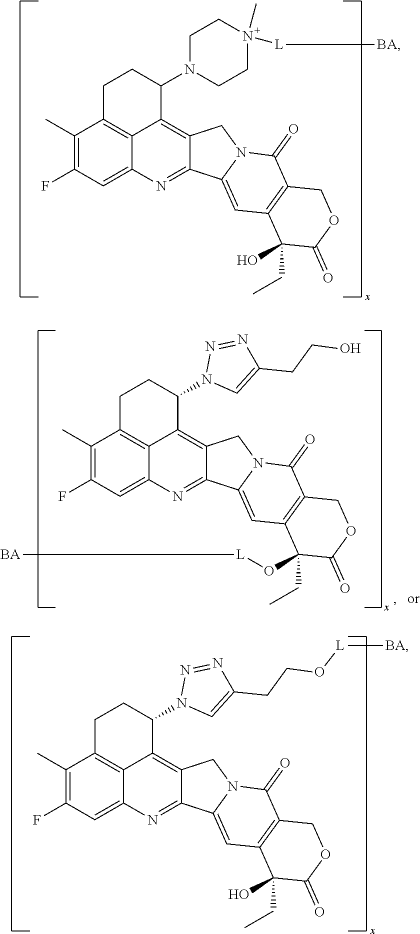

or a pharmaceutically acceptable salt, tautomer, solvate, stereoisomer, enantiomer, isotopologue, or prodrug thereof, wherein values and alternative values for the variables (e.g., L and x) are as described elsewhere herein.

In some embodiments, L is

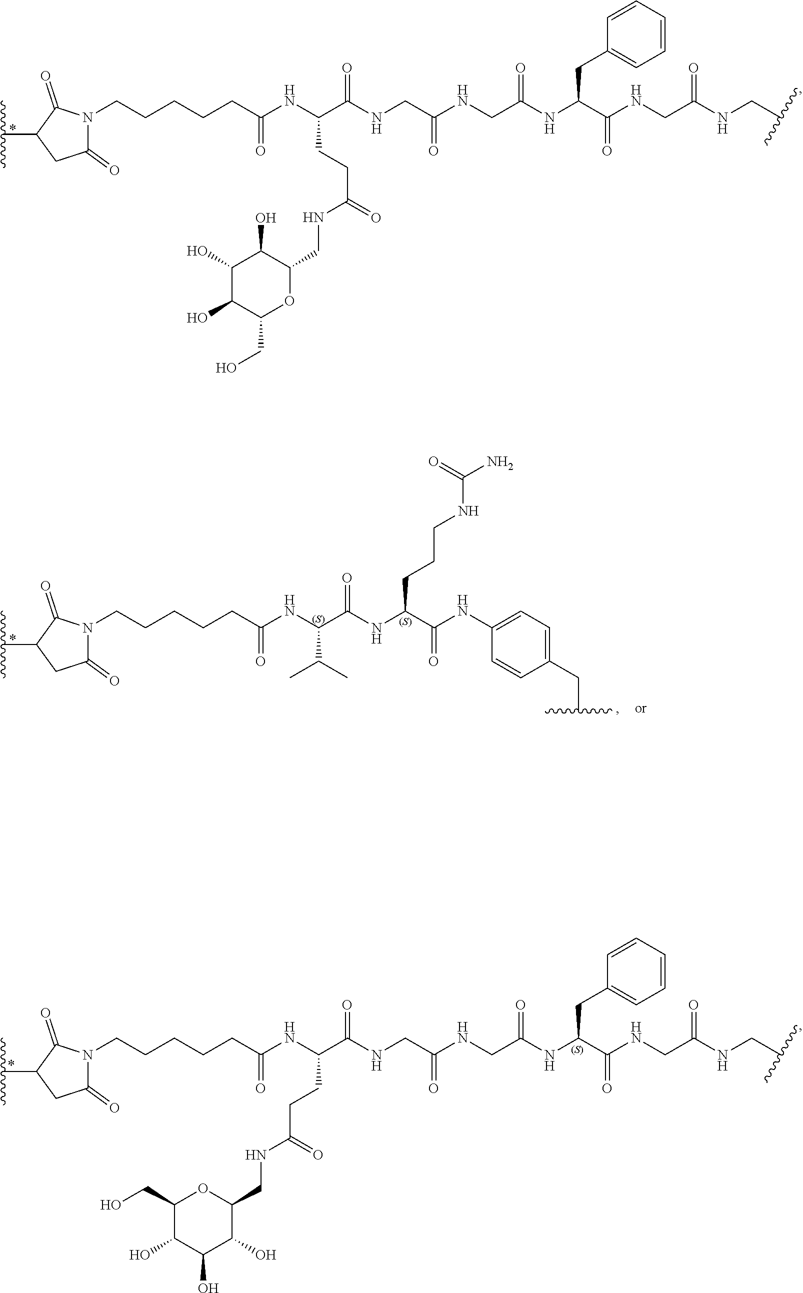

wherein the bond marked with asterisk is connected to BA.

In some embodiments, L is

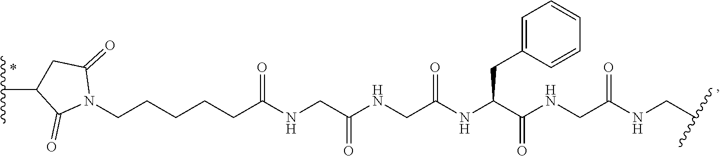 [SMILES: *CNC(=O)CNC(=O)[C@H](Cc1ccccc1)NC(=O)CNC(=O)CNC(=O)CCCCCN1C(=O)CC(*)C1=O]

wherein the bond marked with asterisk is connected to BA.

In some embodiments, L is:

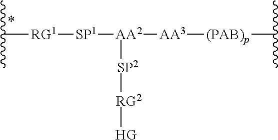

wherein values and alternative values for the variables (e.g., RG¹, SP¹, AA², AA, PAB, p, SP² RG², and HG) are as described elsewhere herein.

In some embodiments, L is:

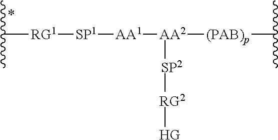

wherein values and alternative values for the variables (e.g., RG¹, SP¹, AA¹, AA², PA, p, SP² RG², and HG) are as described elsewhere herein.

In some embodiments, L is:

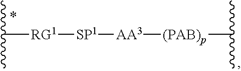

wherein values and alternative values for the variables (e.g., RG¹, SP¹, AA³, PAB, and p) are as described elsewhere herein.

In some embodiments, -AA²(SP²-RG²-HG)-AA³-(PAB)ₚ— is

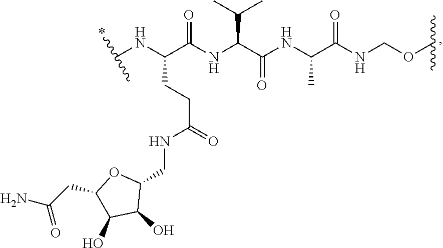

wherein * marks the bond that connects to SP¹.

In some embodiments, an antibody drug conjugate is selected from the following, or a pharmaceutically acceptable salt, tautomer, solvate, stereoisomer, enantiomer, or isotopologue thereof, wherein Ab is any of the anti-B37H3 antibodies disclosed herein:

| Examples | Structures |
|---|---|
| ADC-P1 BGB-C-269 | 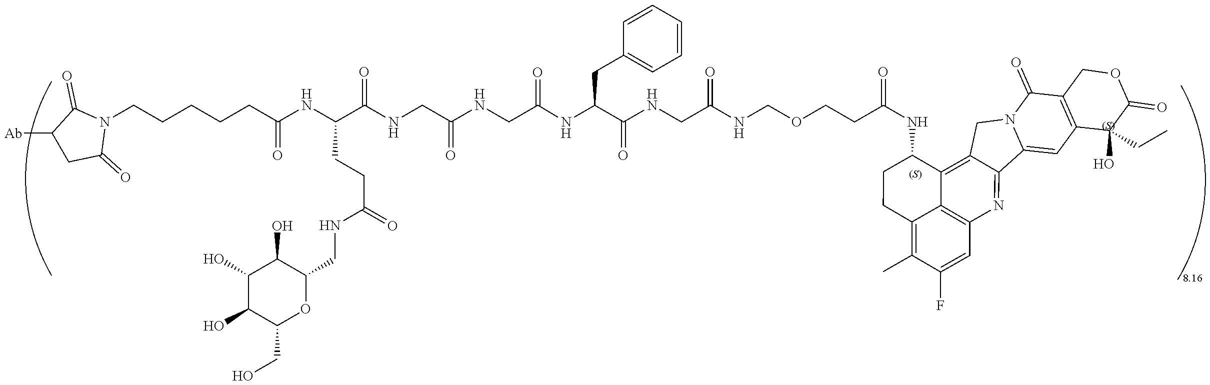 |
| ADC-P2 BGB-C-276 | 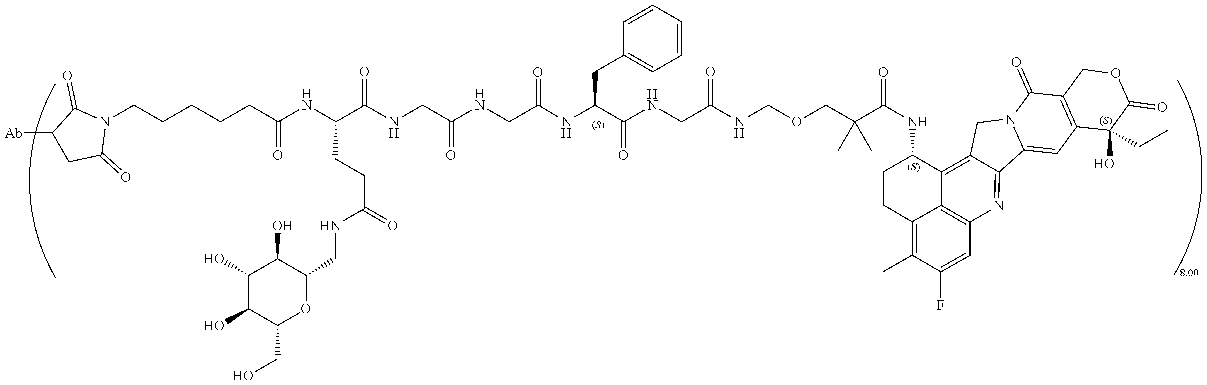 |
| ADC-P3 BGB-C-290 | 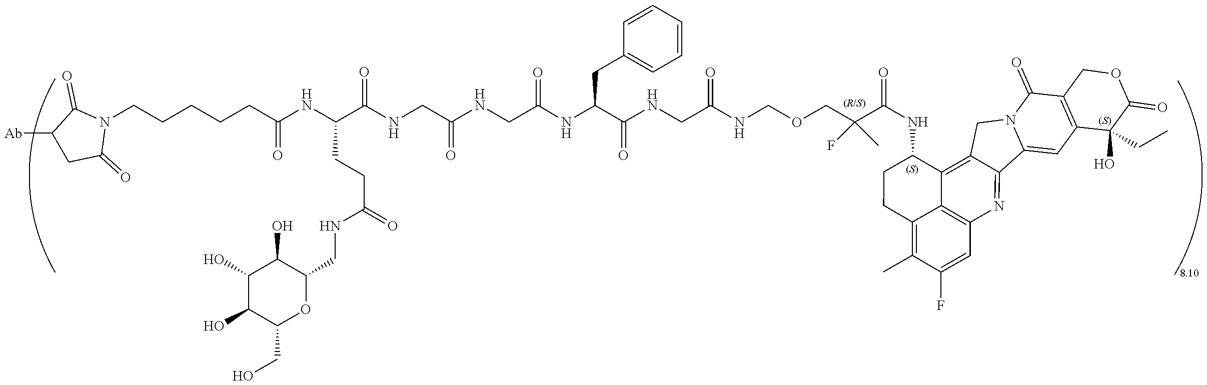 |
| ADC-1 BGB-C-81 | 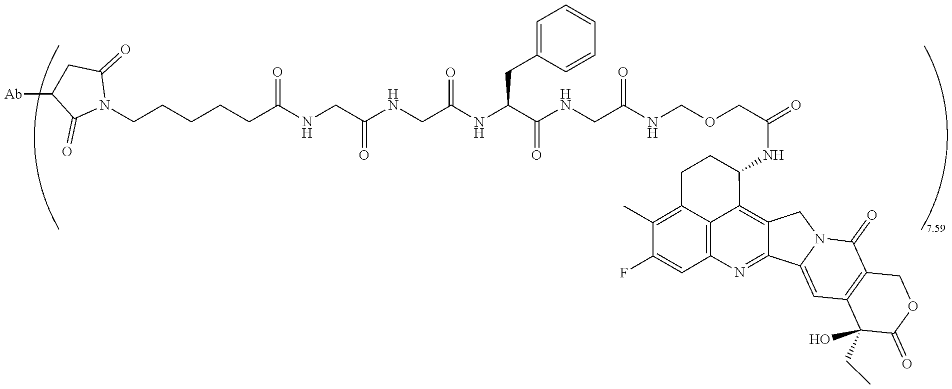 |
| ADC-2 BGB-C-115 | 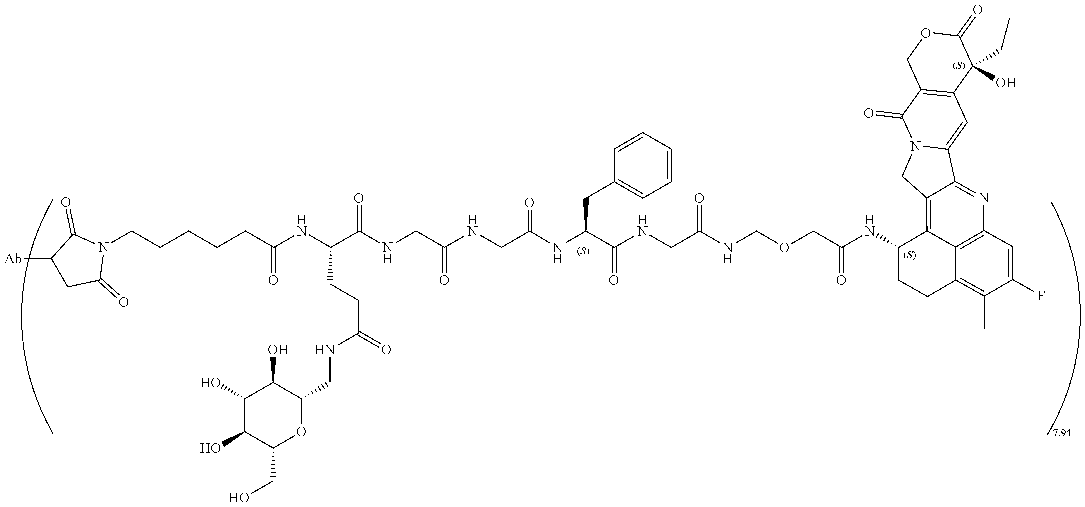 |
| ADC-3 BGB-C-245 | 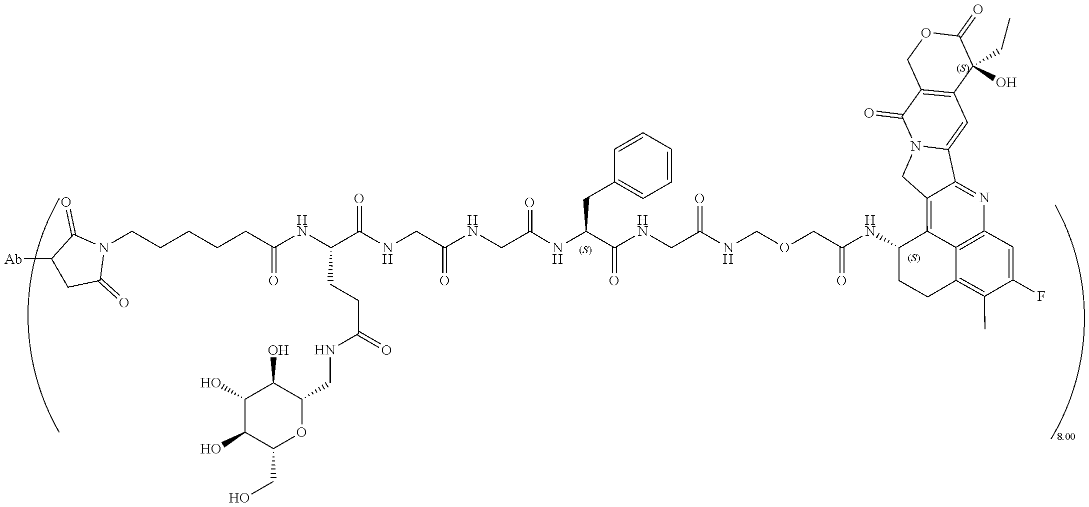 |
| ADC-4 BGB-C-318 | 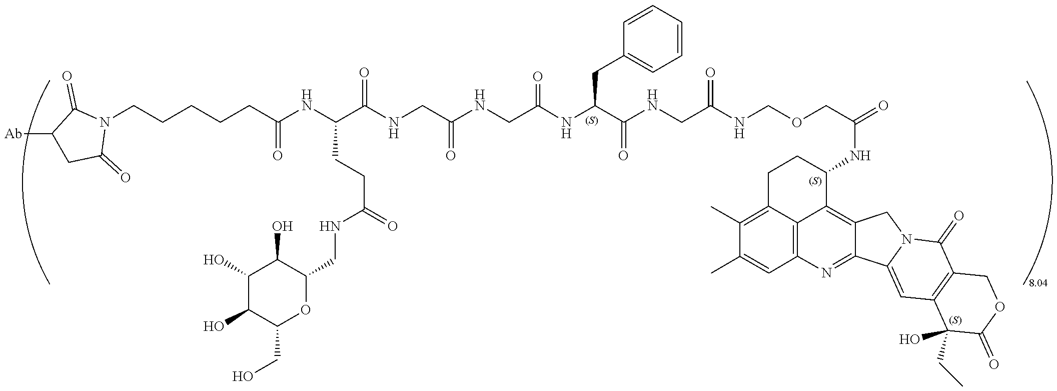 |
| ADC-5 BGB-C-277 | 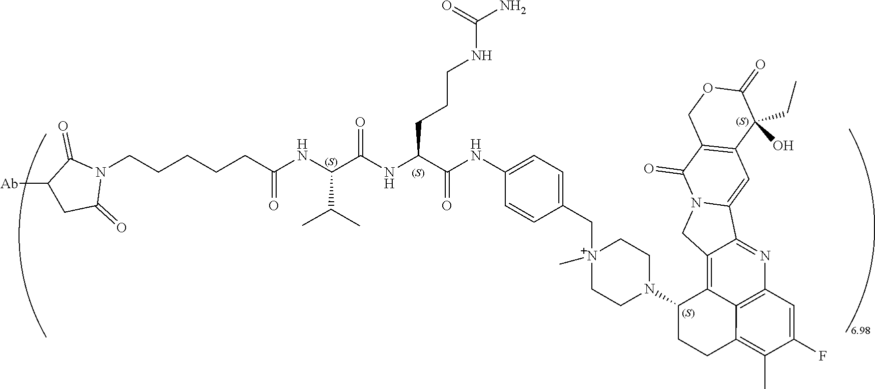 |
| ADC-6 BGB-C-306 | 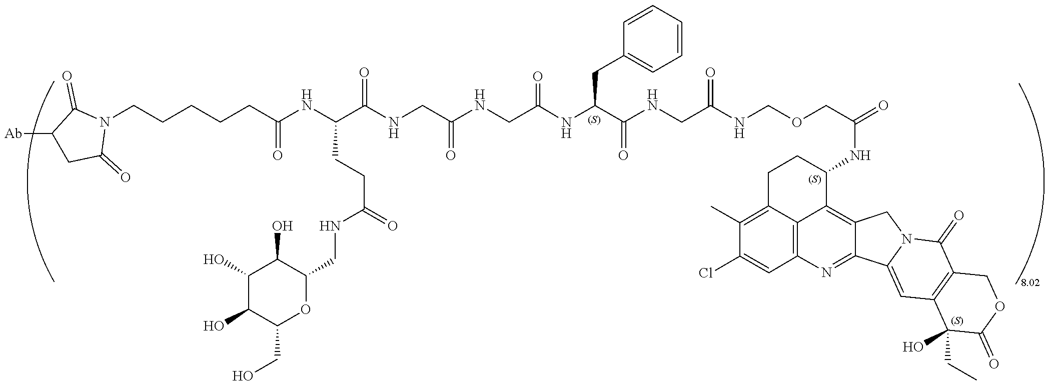 |
| ADC-7 BGB-C-329 | 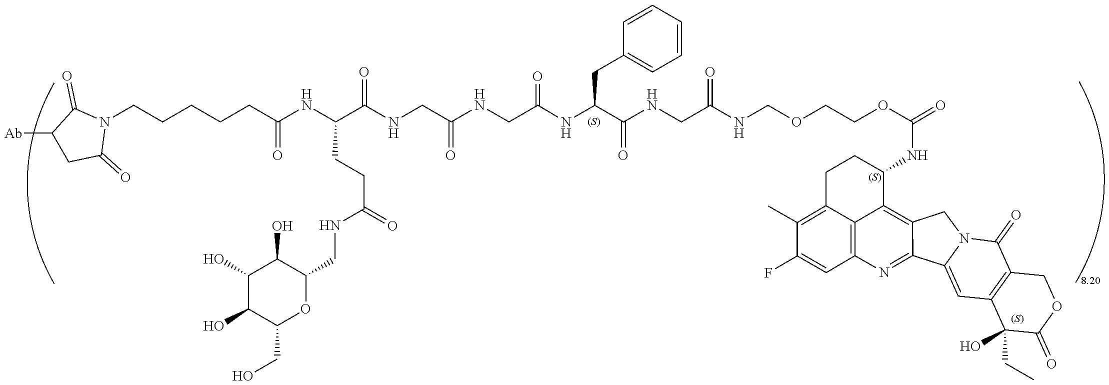 |
| ADC-8 BGB-C-323 | 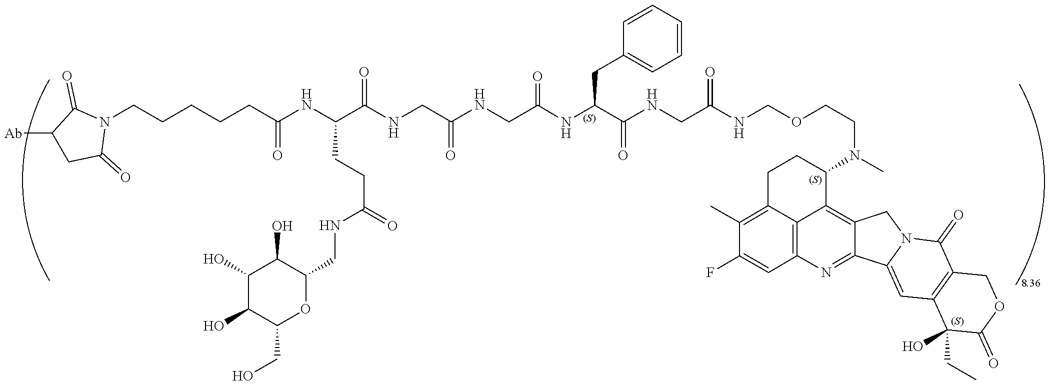 |
| ADC-9 BGB-C-331 | 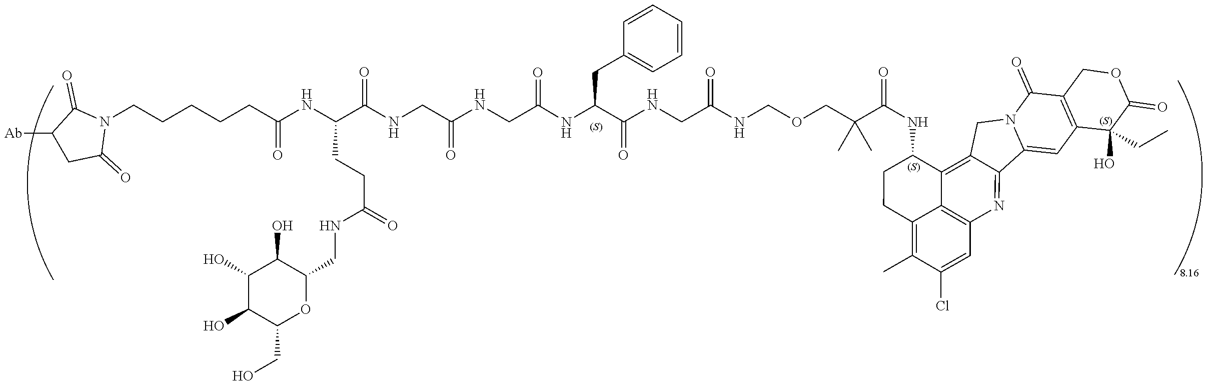 |
| ADC-10 BGB-C-325 | 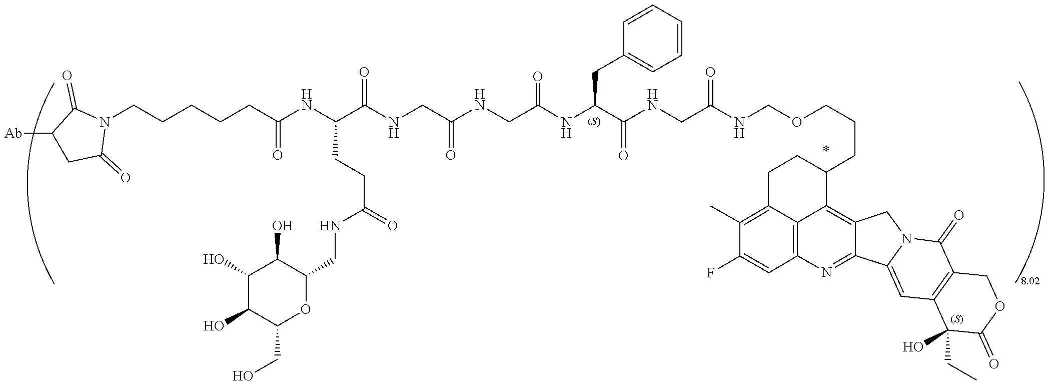 |
| ADC-11 BGB-C-307 | 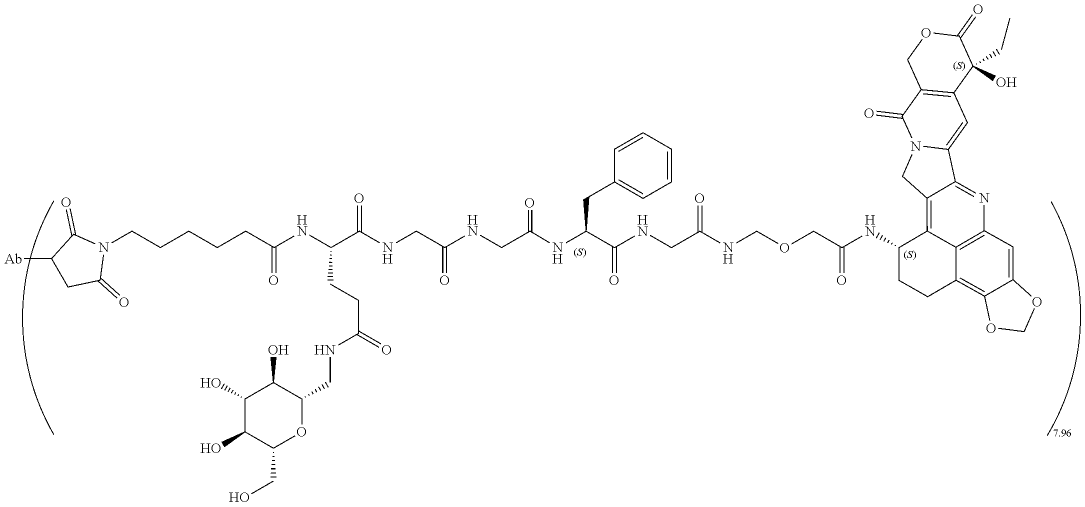 |
| ADC-12 BGB-C-287 | 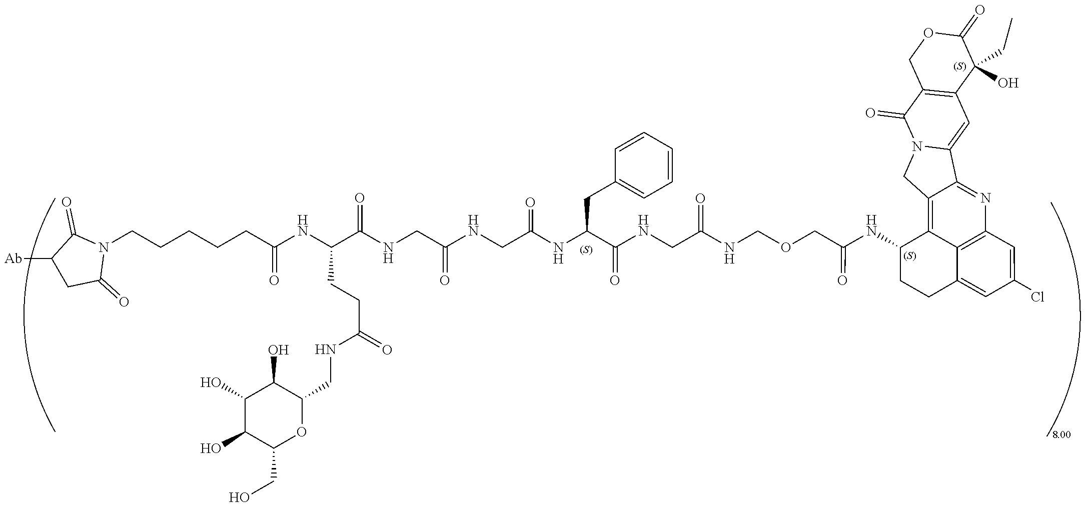 |
| ADC-13 BGB-C-288 | 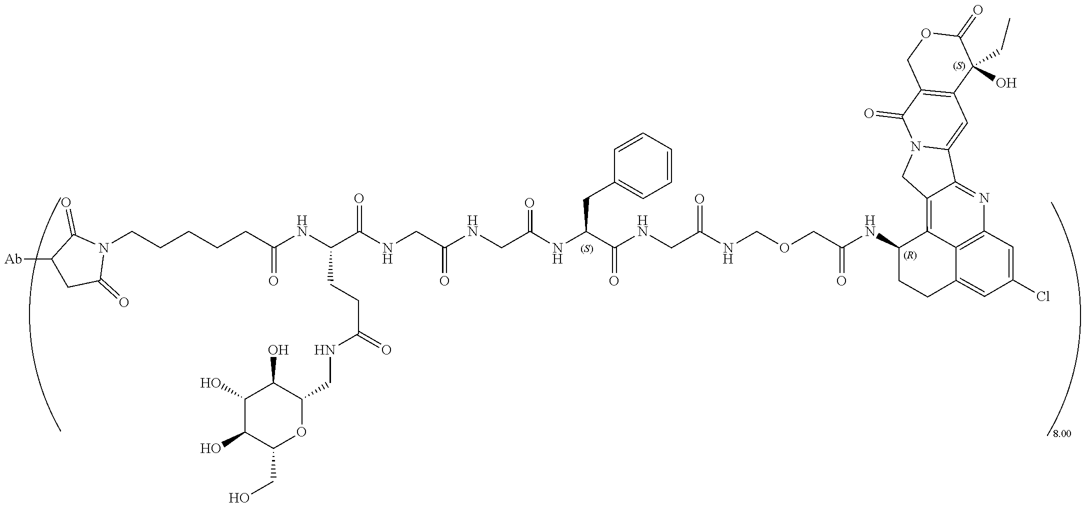 |
| ADC-14 BGB-C-317 | 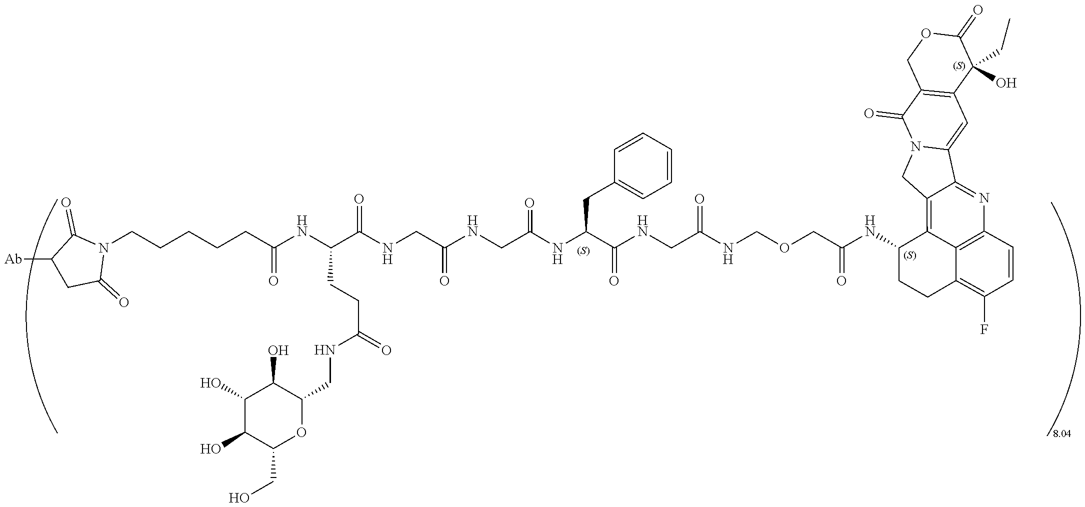 |
| ADC-15 BGB-C-292 | 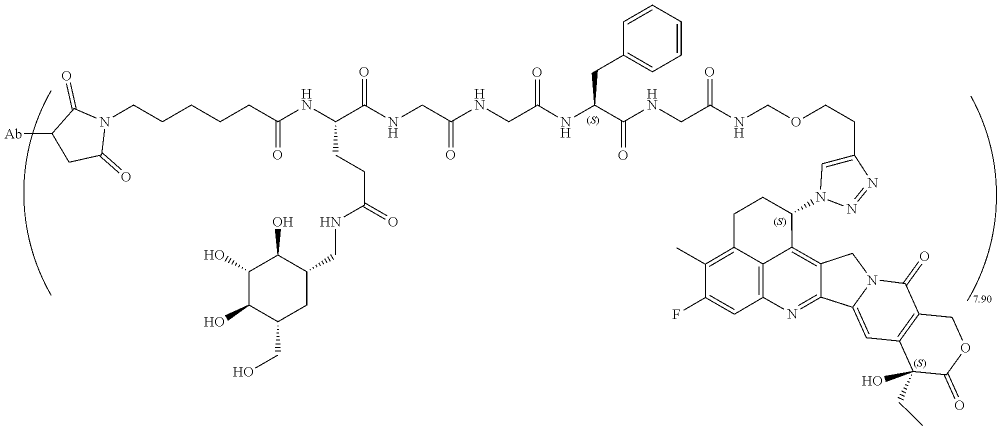 |
| ADC-16 BGB-C-348 | 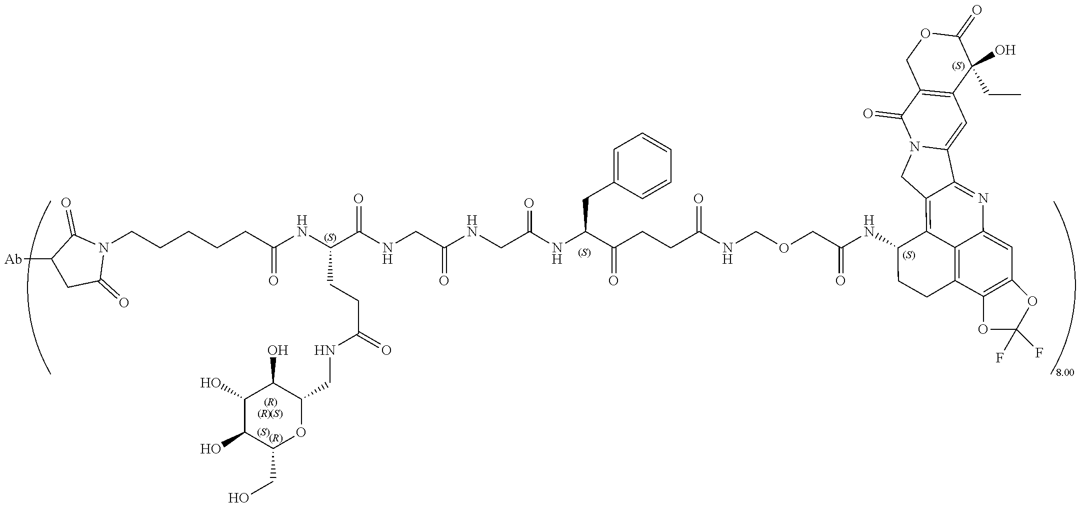 |
| ADC-17 BGB-C-319 | 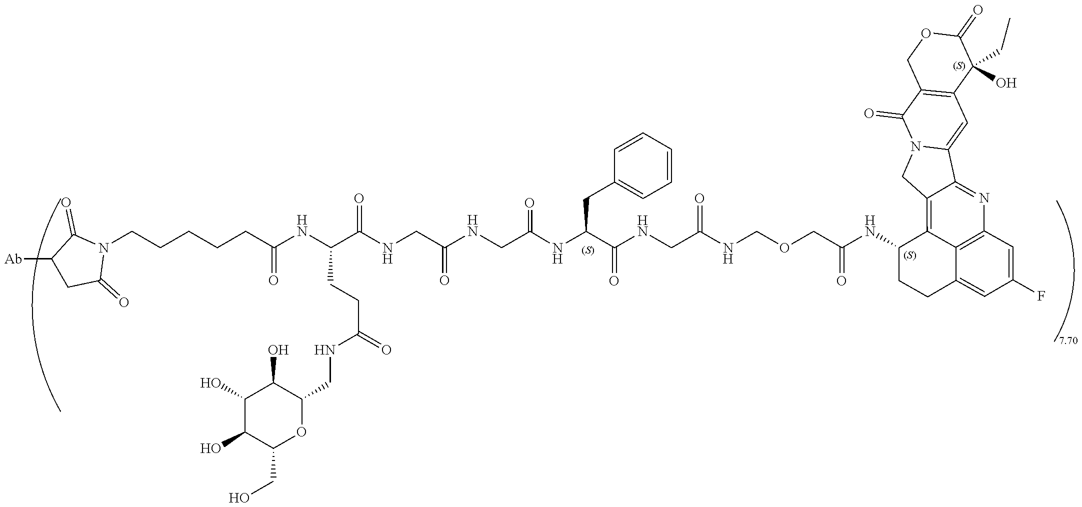 |
| ADC-18 BGB-C-343 | 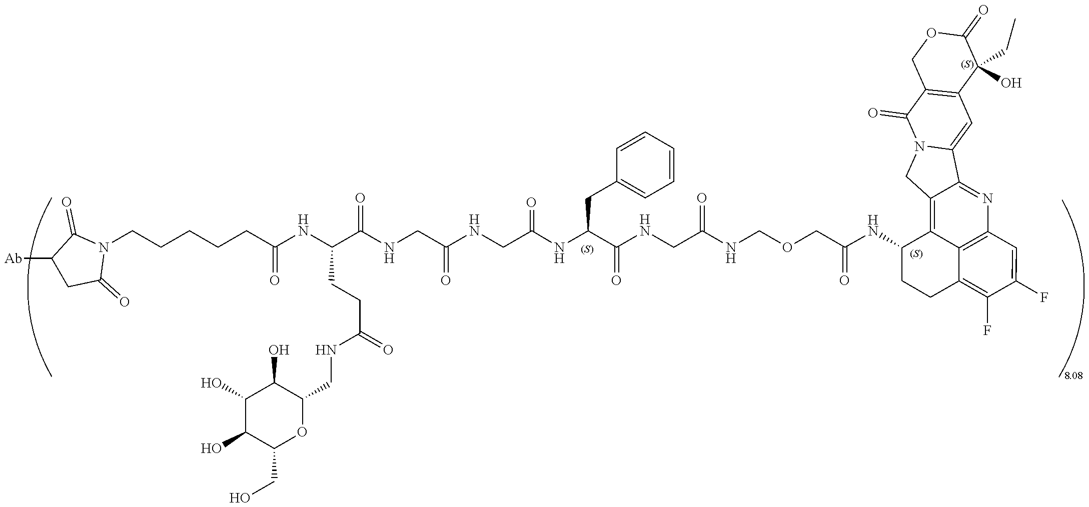 |
| ADC-19 BGB-C-302 | 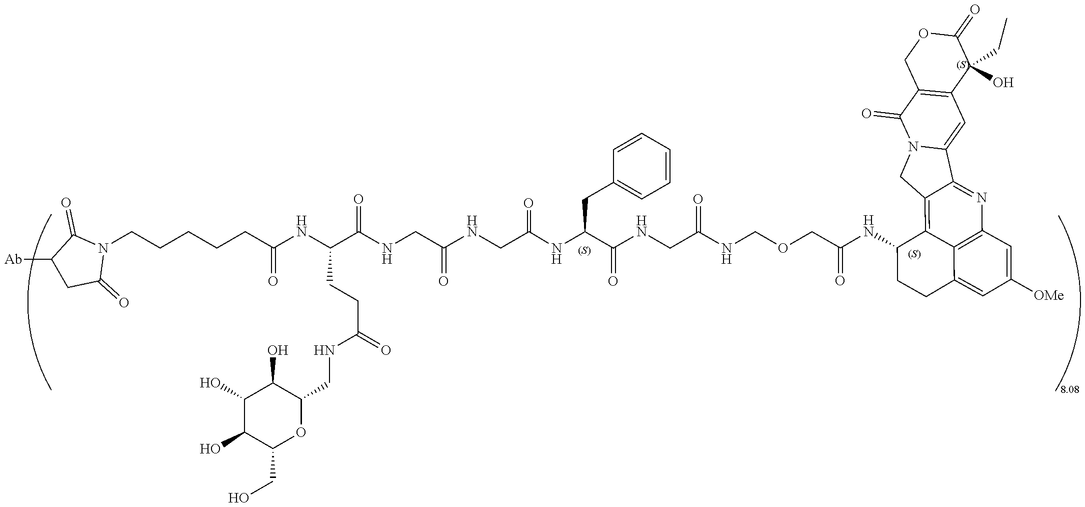 |
| ADC-20 BGB-C-320 | 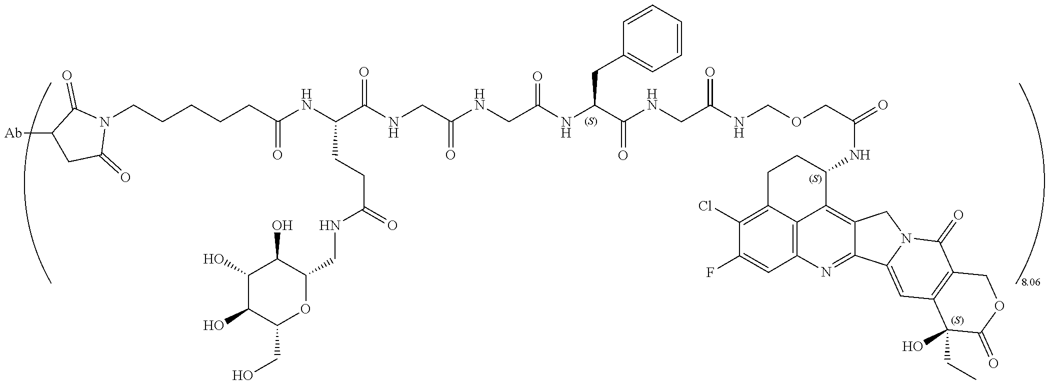 |
| ADC-21 BGB-C-330 | 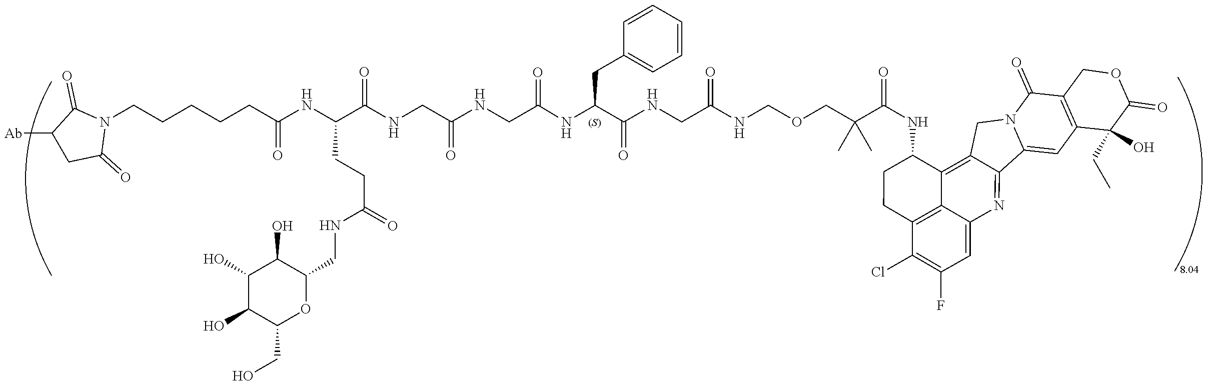 |
| ADC-22 BGB-C-346 | 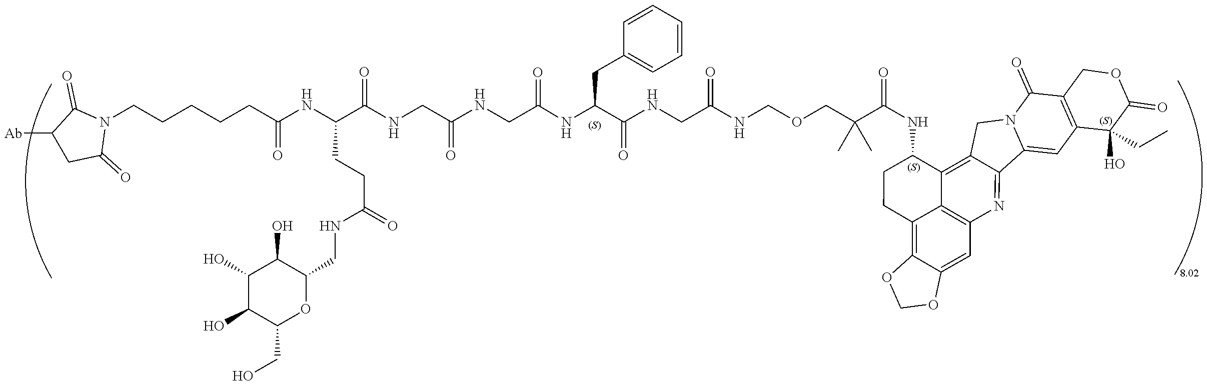 |
| ADC-23 BGB-C-358 | 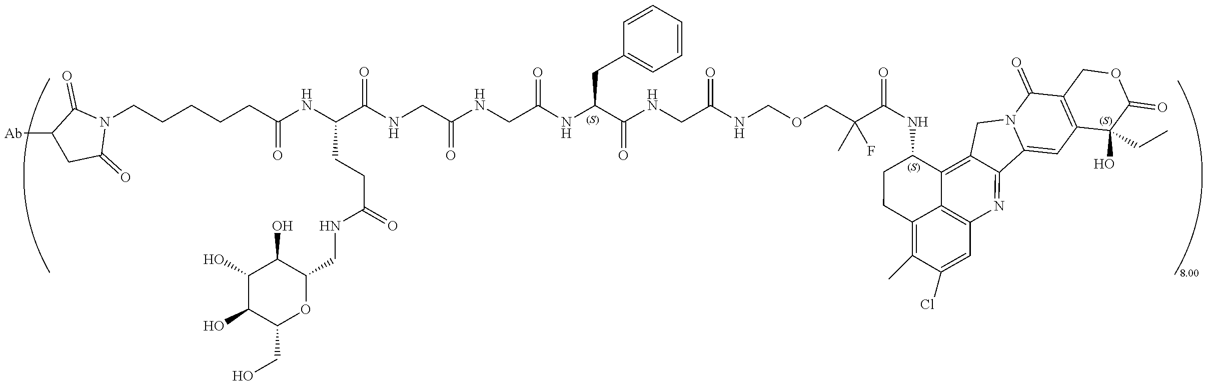 |
| ADC-24 BGB-C-373 | 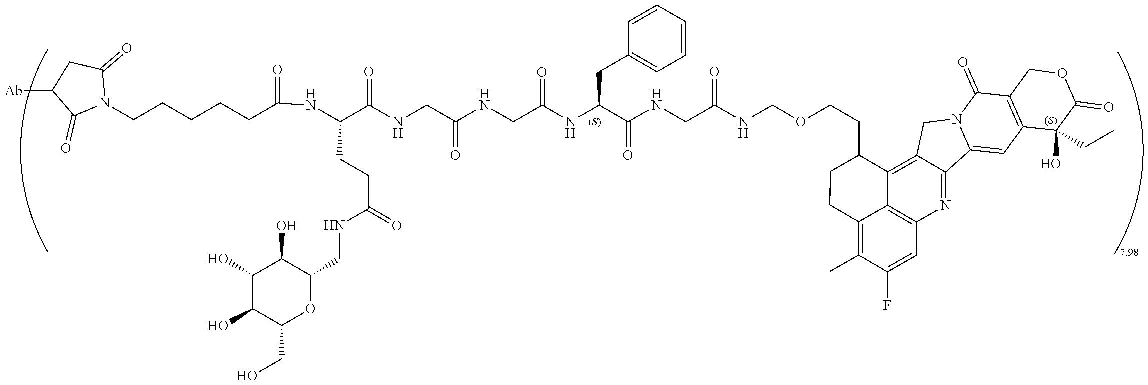 |
| ADC-25 BGB-C-421 | 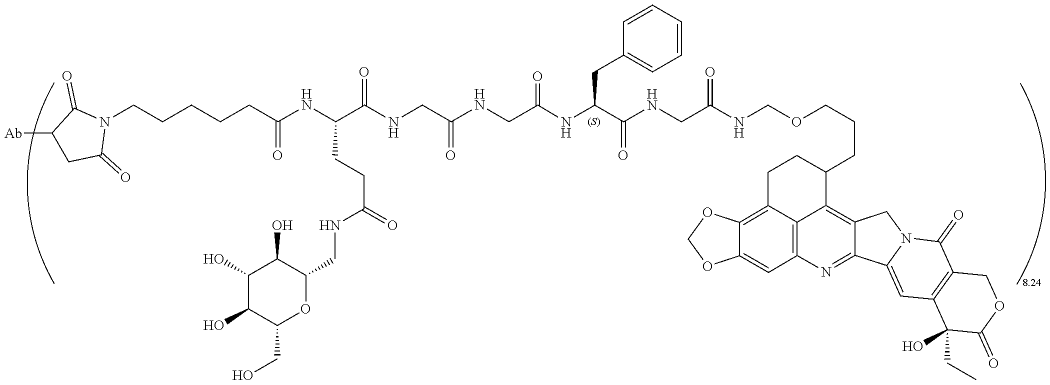 |
| ADC-26 BGB-C-395 | 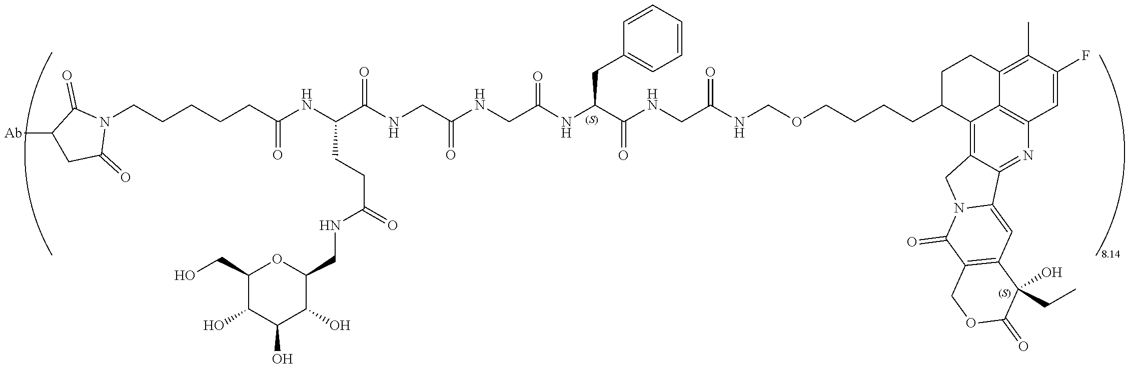 |

In some embodiments, the antibody or antigen binding fragment thereof comprises:
(i) a heavy chain variable region (VH) that comprises (a) a HCDR1 (Heavy Chain Complementarity Determining Region 1) of SEQ ID NO: 11, (b) a HCDR2 of SEQ ID NO: 2, and (c) a HCDR3 of SEQ HD NO: 14; and a light chain variable region (VL) that comprises (d) a LCDR1 (Light Chain Complementarity Determining Region 1) of SEQ ID NO: 23, (e) a LCDR2 of SEQ ID NO: 5, and (f) a LCDR3 of SEQ ID NO: 6;
(ii) a heavy chain variable region (VH) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises (d) a LCDR1 of SEQ ID NO: 4, (e) a LCDR2 of SEQ ID NO: 5, and (f) a LCDR3 of SEQ ID NO: 6;
(iii) a heavy chain variable region (VH) that comprises (a) a HCDR1 of SEQ ID NO: 11, (b) a HCDR2 of SEQ ID NO: 2, and (c) a HCDR3 of SEQ ID NO: 3; and a light chain variable region (VL) that comprises (d) a LCDR1 of SEQ ID NO: 4, (e) a LCDR2 of SEQ ID NO: 5, and (f) a LCDR3 of SEQ ID NO: 6;
(iv) a heavy chain variable region (VH) that comprises (a) a HCDR1 of SEQ ID NO: 1, (b) a HCDR2 of SEQ ID NO: 2, and (c) a HCDR3 of SEQ ID NO: 14; and a light chain variable region (VL) that comprises (d) a LCDR1 of SEQ ID NO: 4, (e) a LCDR2 of SEQ ID NO: 5, and (f) a LCDR3 of SEQ ID NO: 6;
(v) a heavy chain variable region (VH) that comprises (a) a HCDR1 of SEQ ID NO: 1, (b) a HCDR2 of SEQ ID NO: 2, and (c) a HCDR3 of SEQ ID NO: 17; and a light chain variable region (VL) that comprises (d) a LCDR1 of SEQ ID NO: 4, (e) a LCDR2 of SEQ ID NO: 5, and (f) a LCDR3 of SEQ ID NO: 6;
(vi) a heavy chain variable region (VH) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises (d) a LCDR1 of SEQ ID NO: 20, (e) a LCDR2 of SEQ ID NO: 5, and (f) a LCDR3 of SEQ ID NO: 6;
(vii) a heavy chain variable region (VH) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises (d) a LCDR1 of SEQ ID NO: 23, (e) a LCDR2 of SEQ ID NO: 5, and (f) a LCDR3 of SEQ ID NO: 6;
(viii) a heavy chain variable region (VH) that comprises (a) a HCDR1 of SEQ ID NO: 11, (b) a HCDR2 of SEQ ID NO: 2, and (c) a HCDR3 of SEQ ID NO: 28; and a light chain variable region (VL) that comprises (d) a LCDR1 of SEQ ID NO: 23, (e) a LCDR2 of SEQ ID NO: 5, and (f) a LCDR3 of SEQ ID NO: 6; or
(ix) a heavy chain variable region (VH) that comprises (a) a HCDR1 of SEQ ID NO: 300, (b) a HCDR2 of SEQ ID NO: 1700 and (c) a HCDR3 of SEQ ID NO: 500 and a light chain variable region (VL) that comprises (d) a LCDR1 of SEQ ID NO: 600, (e) a LCDR2 of SEQ ID NO: 700, and (f) a LCDR3 of SEQ ID NO: 800.

In some embodiments, the antibody or antigen-binding fragment comprises: (i) a heavy chain variable region comprising an amino acid sequence at least 90, 91, 92, 93, 94, 95, 96, 97, 98 or 99% identical to SEQ ID NO: 26, and a light chain variable region comprising an amino acid sequence at least 90, 91, 92, 93, 94, 95, 96, 97, 98 or 99% identical to SEQ ID NO: 24;
(ii) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 8;
(iii) a heavy chain variable region comprising an amino acid sequence at least 90, 91, 92, 93, 94, 95, 96, 97, 98 or 99% identical to SEQ ID NO: 12, and a light chain variable region comprising an amino acid sequence at least 90, 91, 92, 93, 94, 95, 96, 97, 98 or 99% identical to SEQ ID NO: 8;
(iv) a heavy chain variable region comprising an amino acid sequence at least 90, 91, 92, 93, 94, 95, 96, 97, 98 or 99% identical to SEQ ID NO: 15, and a light chain variable region comprising an amino acid sequence at least 90, 91, 92, 93, 94, 95, 96, 97, 98 or 99% identical to SEQ ID NO: 8;
(v) a heavy chain variable region comprising an amino acid sequence at least 90, 91, 92, 93, 94, 95, 96, 97, 98 or 99% identical to SEQ ID NO: 18, and a light chain variable region comprising an amino acid sequence at least 90, 91, 92, 93, 94, 95, 96, 97, 98 or 99% identical to SEQ ID NO: 8;
(vi) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 21;
(vii) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 24;
(viii) a heavy chain variable region comprising an amino acid sequence at least 90, 91, 92, 93, 94, 95, 96, 97, 98 or 99% identical to SEQ ID NO: 29, and a light chain variable region comprising an amino acid sequence at least 90, 91, 92, 93, 94, 95, 96, 97, 98 or 99% identical to SEQ ID NO: 24;
(ix) a heavy chain variable region comprising an amino acid sequence at least 90, 91, 92, 93, 94, 95, 96, 97, 98 or 99% identical to SEQ ID NO: 1800, and a light chain variable region comprising an amino acid sequence at least 90, 91, 92, 93, 94, 95, 96, 97, 98 or 99% identical to SEQ ID NO: 1400;
(viii) a heavy chain variable region comprising an amino acid sequence at least 90, 91, 92, 93, 94, 95, 96, 97, 98 or 99% identical to SEQ ID NO: 900, and a light chain variable region comprising an amino acid sequence at least 90, 91, 92, 93, 94, 95, 96, 97, 98 or 99% identical to SEQ ID NO: 1000; or
(ix) a heavy chain variable region comprising an amino acid sequence at least 90, 91, 92, 93, 94, 95, 96, 97, 98 or 99% identical to SEQ ID NO: 1300, and a light chain variable region comprising an amino acid sequence at least 90, 91, 92, 93, 94, 95, 96, 97, 98 or 99% identical to SEQ ID NO: 1400.

In some embodiments, one, two, three, four, five, six, seven, eight, nine, or ten amino acids within each of SEQ ID Nos: 26 and 24, each of SEQ ID Nos: 7 and 8, each of SEQ ID Nos: 12 and 8, each of SEQ ID Nos: 15 and 8, each of SEQ ID Nos: 18 and 8, each of SEQ ID Nos: 7 and 21, each of SEQ ID Nos: 7 and 24, each of SEQ ID Nos: 29 and 24, each of SEQ ID Nos: 1800 and 1400, each of SEQ ID Nos: 900 and 1000, or each of SEQ ID Nos: 1300 and 1400 have been inserted, deleted, or substituted in the antibody or antigen-binding fragment.

In some embodiments, the antibody or antigen-binding fragment comprises:
(i) a heavy chain variable region comprising SEQ ID NO: 26, and a light chain variable region comprising SEQ ID NO: 24;
(ii) a heavy chain variable region comprising SEQ ID NO: 7, and a light chain variable region comprising SEQ ID NO: 8;
(iii) a heavy chain variable region comprising SEQ ID NO: 12, and a light chain variable region comprising SEQ ID NO: 8;
(iv) a heavy chain variable region comprising SEQ ID NO: 15, and a light chain variable region comprising SEQ ID NO: 8;
(v) a heavy chain variable region comprising SEQ ID NO: 18, and a light chain variable region comprising SEQ ID NO: 8;
(vi) a heavy chain variable region comprising SEQ ID NO: 7, and a light chain variable region comprising SEQ ID NO: 21;
(vii) a heavy chain variable region comprising SEQ ID NO: 7, and a light chain variable region comprising SEQ ID NO: 24;
(viii) a heavy chain variable region comprising SEQ ID NO: 29, and a light chain variable region comprising SEQ ID NO: 24;
(ix) a heavy chain variable region comprising SEQ ID NO: 900, and a light chain variable region comprising SEQ ID NO: 100;
(x) a heavy chain variable region comprising SEQ ID NO: 1300, and a light chain variable region comprising SEQ ID NO: 1400; or
(xi) a heavy chain variable region comprising SEQ ID NO: 1800, and a light chain variable region comprising SEQ ID NO: 1400.

In some embodiments, the antibody or antigen-binding fragment thereof specifically binds to an epitope comprising, consisting essentially of, or consisting of amino acid residues 29-139 of human 4Ig-B7H3 (SEQ ID NO: 801). In some embodiments, the antibody or antigen-binding fragment thereof specifically binds to an epitope comprising, consisting essentially of or consisting of amino acid residues 243-357 of human 4Ig-B7H3 (SEQ ID NO: 801).

In some embodiments, the antibody or antigen-binding fragment thereof specifically binds to IgV1 domain of human 4Ig-B7H3. In some embodiments, the antibody or antigen-binding fragment thereof specifically binds to IgV2 domain of human 4Ig-B7H3. In some embodiments, the antibody or antigen-binding fragment thereof specifically binds to human 4Ig-B7H3, and binds to both IgV1 and IgV2 domains of human 4Ig-B7H3.

In some embodiments, the antibody or antigen-binding fragment thereof specifically binds to human 4Ig-B7H3, and does not bind to an epitope comprising, consisting essentially of, or consisting of amino acid residues 145-238 of human 4Ig-B7H3 (SEQ ID NO: 801). In some embodiments, the antibody or antigen-binding fragment thereof specifically binds to human 4Ig-B7H3, and does not bind to an epitope comprising, consisting essentially of, or consisting of amino acid residues 363-456 of human 4Ig-B7H1-3 (SEQ ID NO: 801). In some embodiments, the antibody or antigen-binding fragment thereof specifically binds to human 4Ig-B7H3, and does not bind to IgC1 domain of human 4Ig-B7H3. In some embodiments, the antibody or antigen-binding fragment thereof specifically binds to human 4Ig-B7H3, and does not bind to IgC2 domain of human 4Ig-B7H3. In some embodiments, the antibody or antigen-binding fragment thereof does not bind to either IgC1 nor IgC2 domain of human 4Ig-B7H3. In some embodiments, the antibody or antigen-binding fragment thereof specifically binds to an epitope that does not overlap with the epitope of reference antibody DS-7300.

In some embodiments, the antibody or antigen-binding fragment is a monoclonal antibody, a human engineered antibody, a single chain antibody (scFv), a Fab fragment, a Fab′ fragment, or a F(ab′)₂ fragment.

In some embodiments, the antibody or antigen-binding fragment comprises a scFv comprising a VH having the amino acid of SEQ ID NO: 26 and a VL having an amino acid of SEQ ID NO: 24, optionally the VH and VL are connected via an amino acid linker, optionally the amino acid linker is any sequence of SEQ ID NO: 35 to SEQ ID NO: 77.

In some embodiments, the antibody or antigen-binding fragment comprises a scFv having the amino acid sequence of SEQ ID NO: 32.

In some embodiments, the antibody or antigen-binding fragment thereof comprises a heavy chain constant region of the subclass of IgG1, IgG2, IgG3, or IgG4, and/or a light chain constant region of the type of kappa or lambda.

In some embodiments, the antibody or antigen-binding fragment thereof comprises a heavy chain constant region of the subclass of IgG1, and a light chain constant region of the type of kappa.

All possible combinations of anti-B7H3 antibodies, linkers, and payloads are contemplated herein.

The antibody drug conjugates disclosed herein may be produced by any method known in the art. In one example, a host cell that has been transformed by an isolated nucleic acid comprising a sequence encoding an anti-B7H3 antibody or antigen-binding fragment thereof is cultured under suitable culturing conditions. The antibody or antigen-binding fragment thereof is thereby expressed and may be recovered from the cell culture.

The cytotoxic agent is conjugated to the antibody or antigen-binding fragment thereof using a linker disclosed herein to produce an antibody drug conjugate, e.g., using a conjugator disclosed herein.

### 7.2.1. Aspect 1

Provided here is an antibody drug conjugate comprising an antibody or antigen binding fragment thereof; and a cytotoxic agent.

### 7.2.2. Aspect 2

Provided here is an antibody drug conjugate having Formula (I):

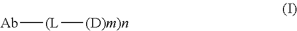

or a pharmaceutically acceptable salt thereof; wherein
Ab is an antibody or antigen binding fragment thereof that binds B7H3, e.g., an antibody or antigen binding fragment thereof capable of specific binding to human B7H3;
L is a linker;
D is a residue of a cytotoxic agent;
m is an integer from 1 to 8; and
n is from 1 to 10.

In one embodiment, in is 1.

In one embodiment, n is from 3 to 10, e.g., from 4 to 10, from 5 to 10, from 6 to 10, or from 7 to 9. In certain embodiments, n is about 8. In embodiments, n is 3, 4, 5, 6, 7, 8, 9, or 10.

In one embodiment, the antibody drug conjugate has Formula (II):

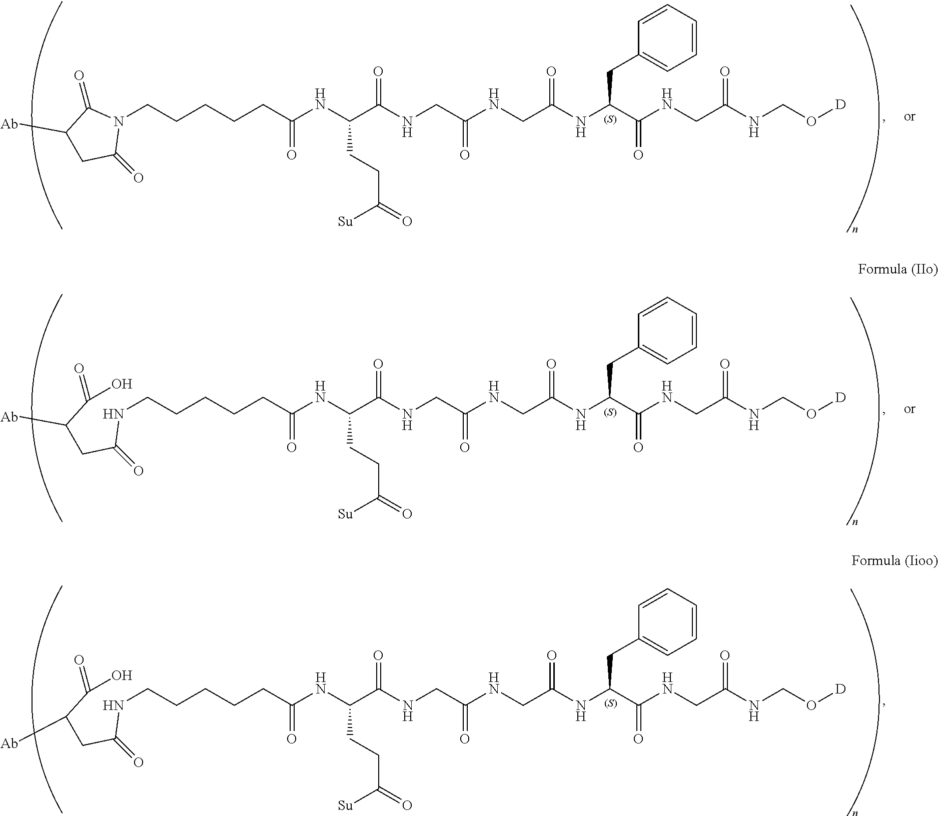

wherein Su is a hydrophilic residue.

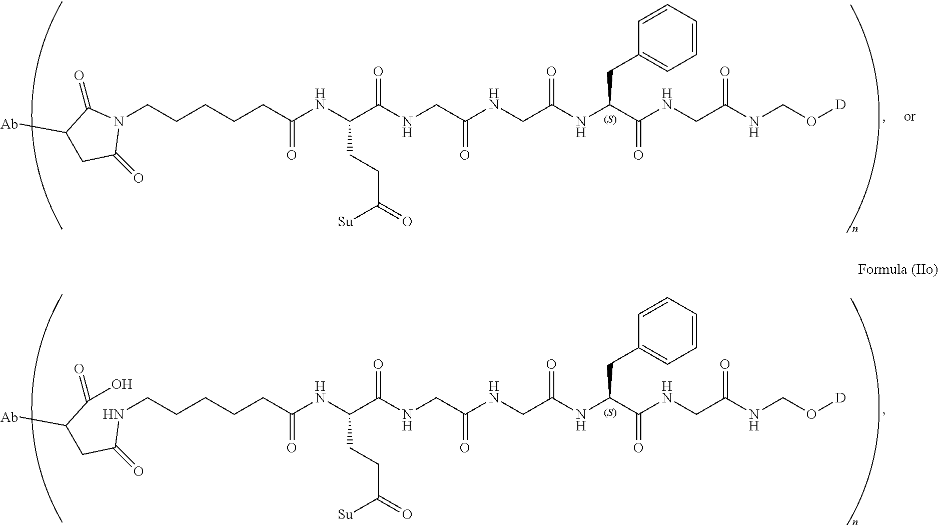

wherein Su is a hydrophilic residue.

In one embodiment, Su is

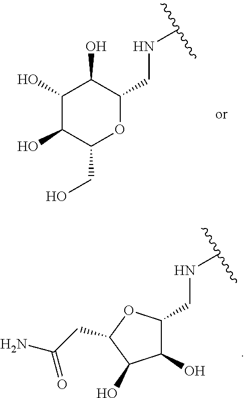 [SMILES: *NC[C@@H]1O[C@H](CO)[C@@H](O)[C@H](O)[C@H]1O ; *NC[C@H]1O[C@@H](CC(N)=O)[C@H](O)[C@@H]1O]

In one embodiment, the antibody drug conjugate has

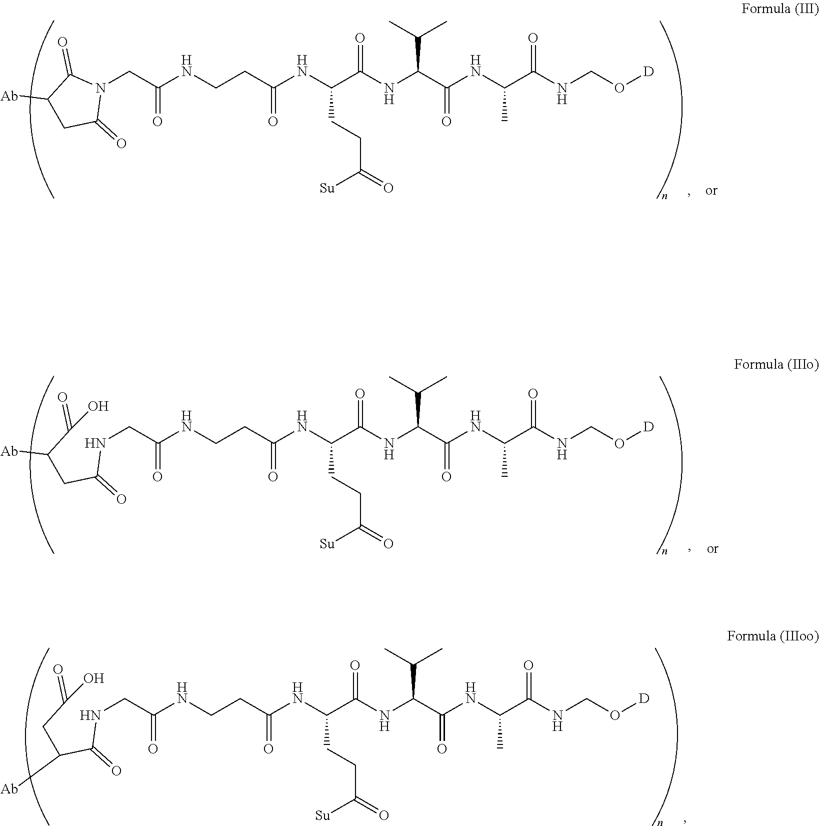

wherein Su is a hydrophilic residue.

In embodiment, the antibody drug conjugate has formula (III):

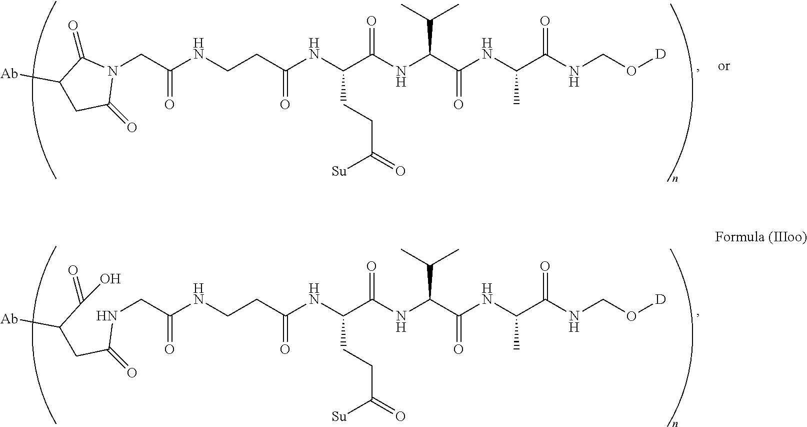

wherein Su is a hydrophilic residue.

In one embodiment, Su is

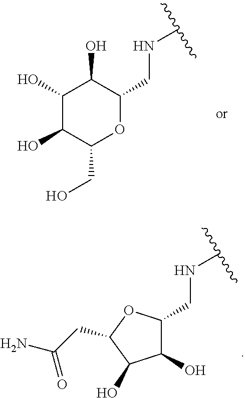 [SMILES: *NC[C@@H]1O[C@H](CO)[C@@H](O)[C@H](O)[C@H]1O ; *NC[C@H]1O[C@@H](CC(N)=O)[C@H](O)[C@@H]1O]

In one embodiment, D is

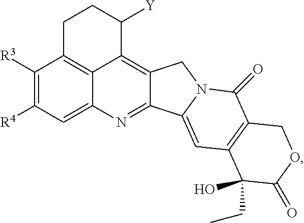

wherein
Y is -A-B—C′-D′ wherein * marks the bond where D connects to the antibody-drug conjugate;
A is a bond, CR¹R², or N—R¹;
B is a bond, —C(═O)—, or —C(═O)O—;
C is a bond, or a divalent group, wherein the divalent group is unsubstituted or substituted C₁₋₈ alkyl, unsubstituted or substituted cycloalkyl, unsubstituted or substituted heterocyclyl, unsubstituted or substituted aryl, or unsubstituted or substituted heteroaryl;
D′ is a bond, NH, or 0;
each of R¹ and R² is, independently, hydrogen, halogen, substituted or unsubstituted alkyl, or substituted or unsubstituted alkoxyl; or R¹ and R² together with the atom to which they are attached form unsubstituted or substituted cycloalkyl, unsubstituted or substituted heterocyclyl, unsubstituted or substituted aryl, or unsubstituted or substituted heteroaryl;
each of R³ and R⁴ is, independently, hydrogen, halogen, substituted or unsubstituted alkyl, or substituted or unsubstituted alkoxyl; or R³ and R⁴ together with the atoms to which they are attached form unsubstituted or substituted cycloalkyl, unsubstituted or substituted heterocyclyl, unsubstituted or substituted aryl, or unsubstituted or substituted heteroaryl.

In one embodiment, D is

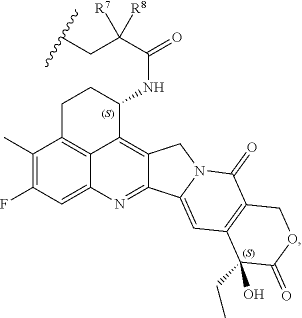

wherein R⁷ and R^{8′} are each independently hydrogen, halogen, or alkyl.

In one embodiment, D is

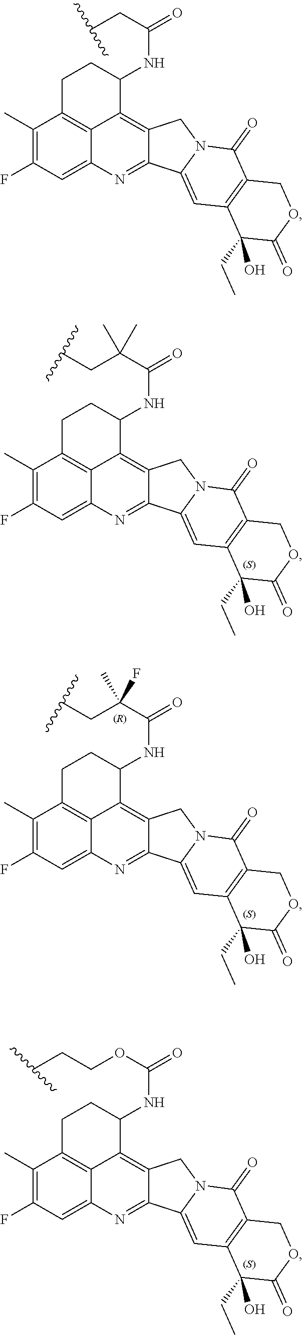

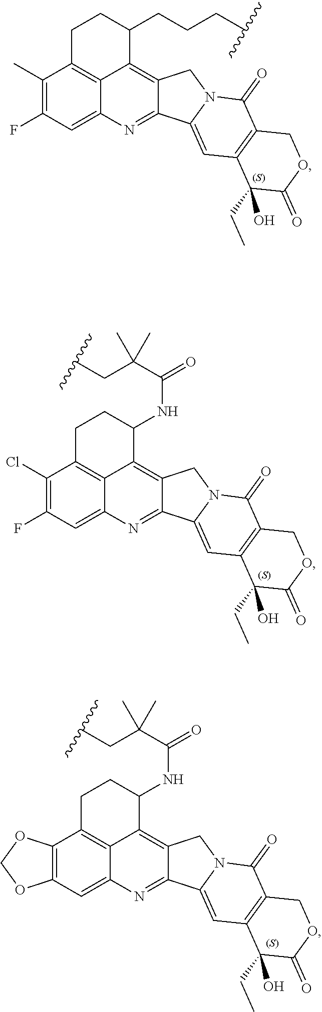

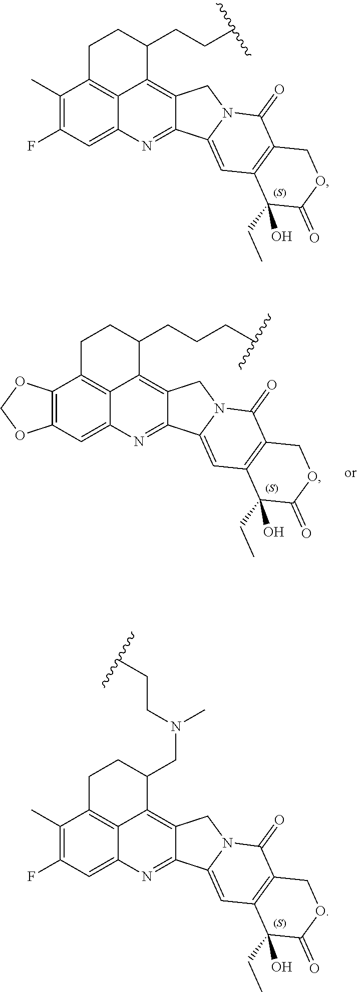

In one embodiment, D is

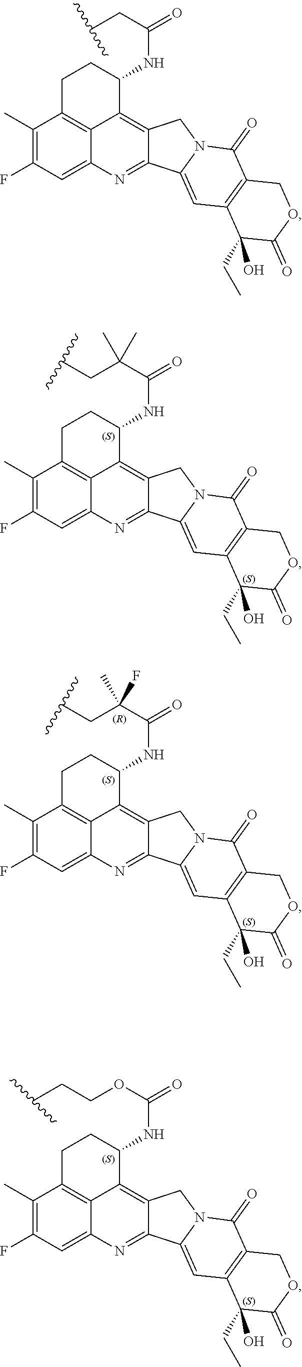

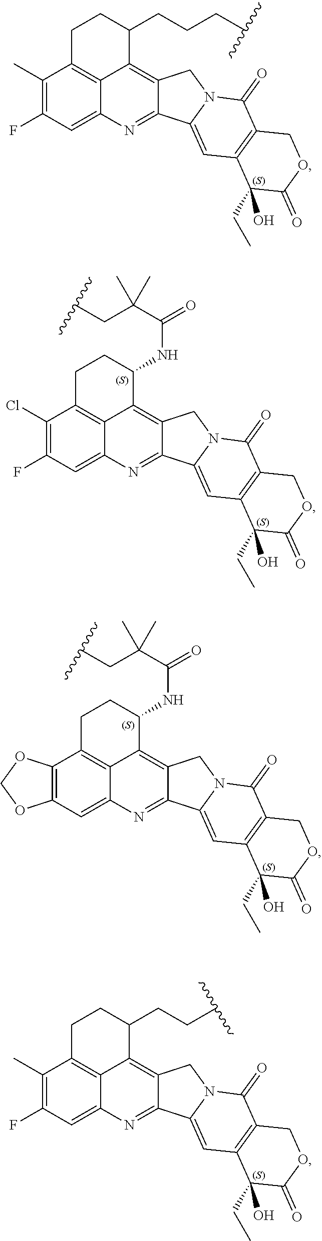

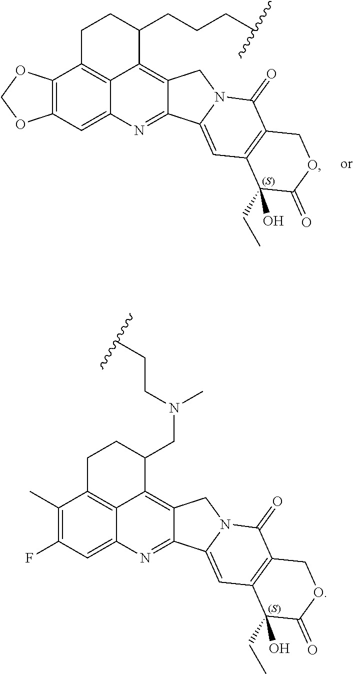

In one embodiment, an antibody drug conjugate has one of the following structures:

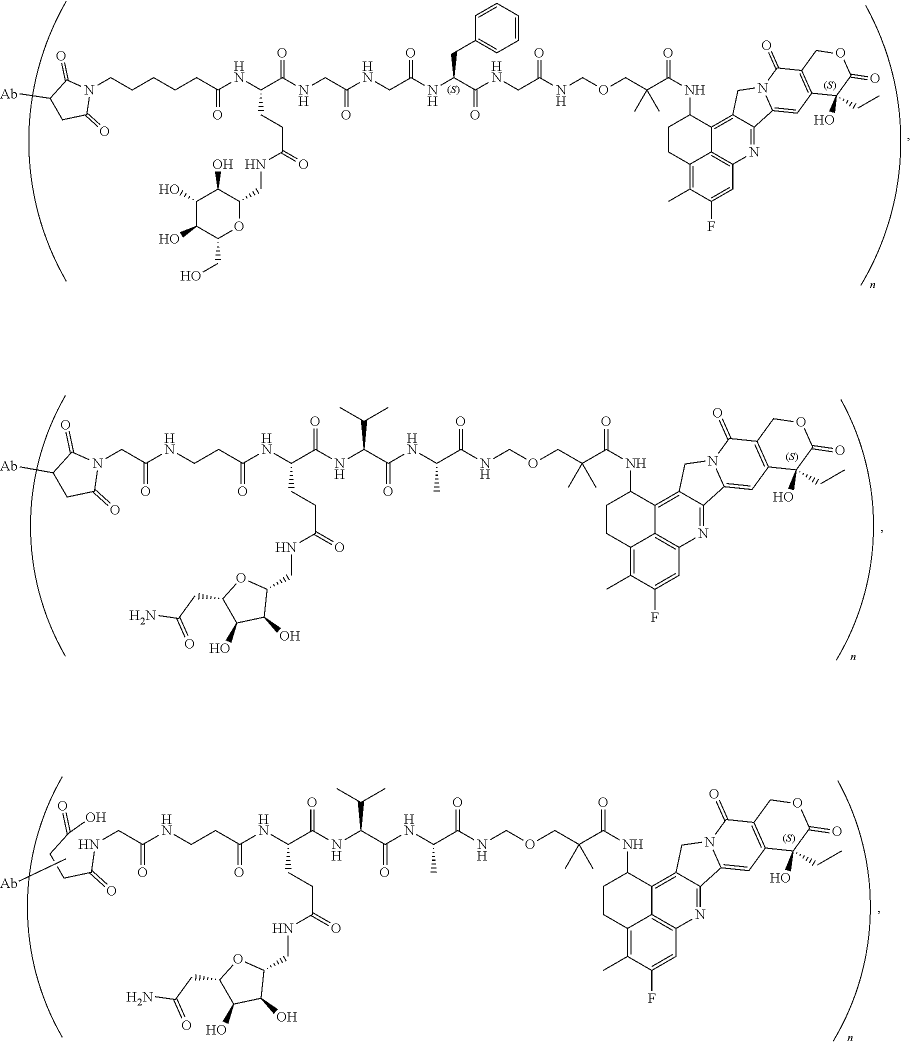

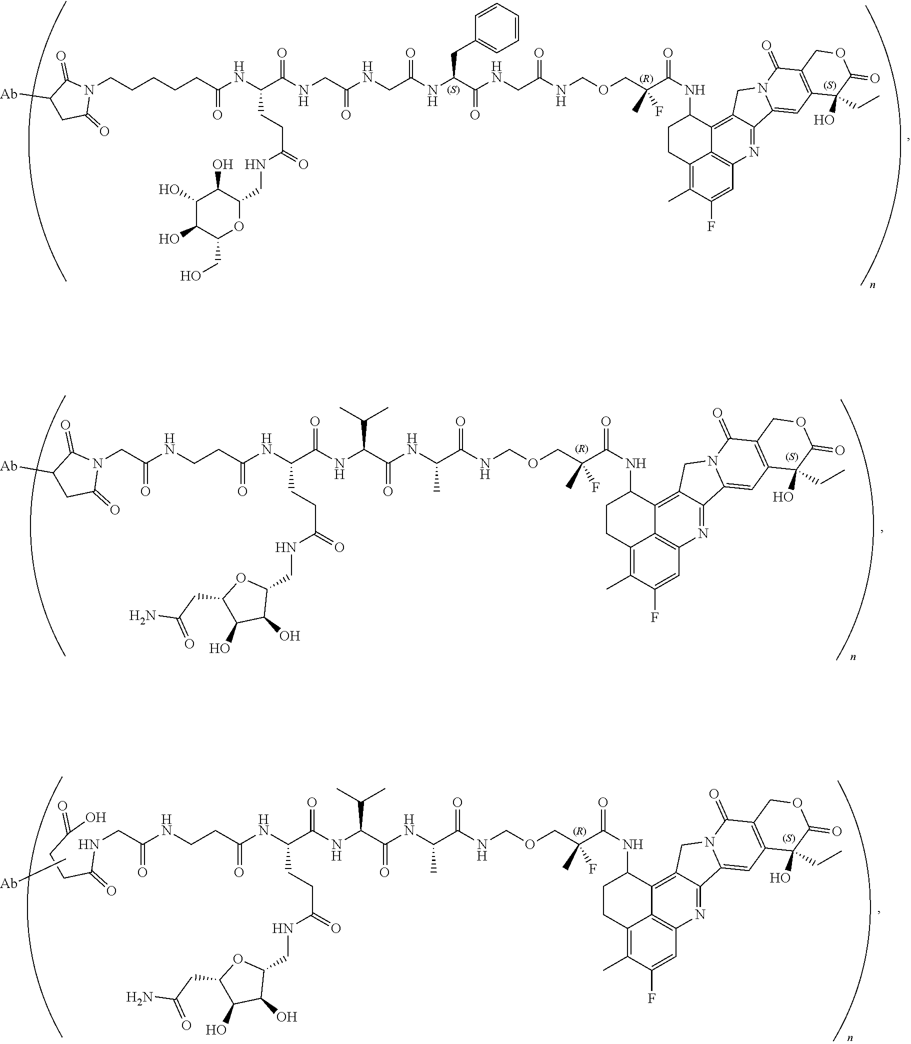

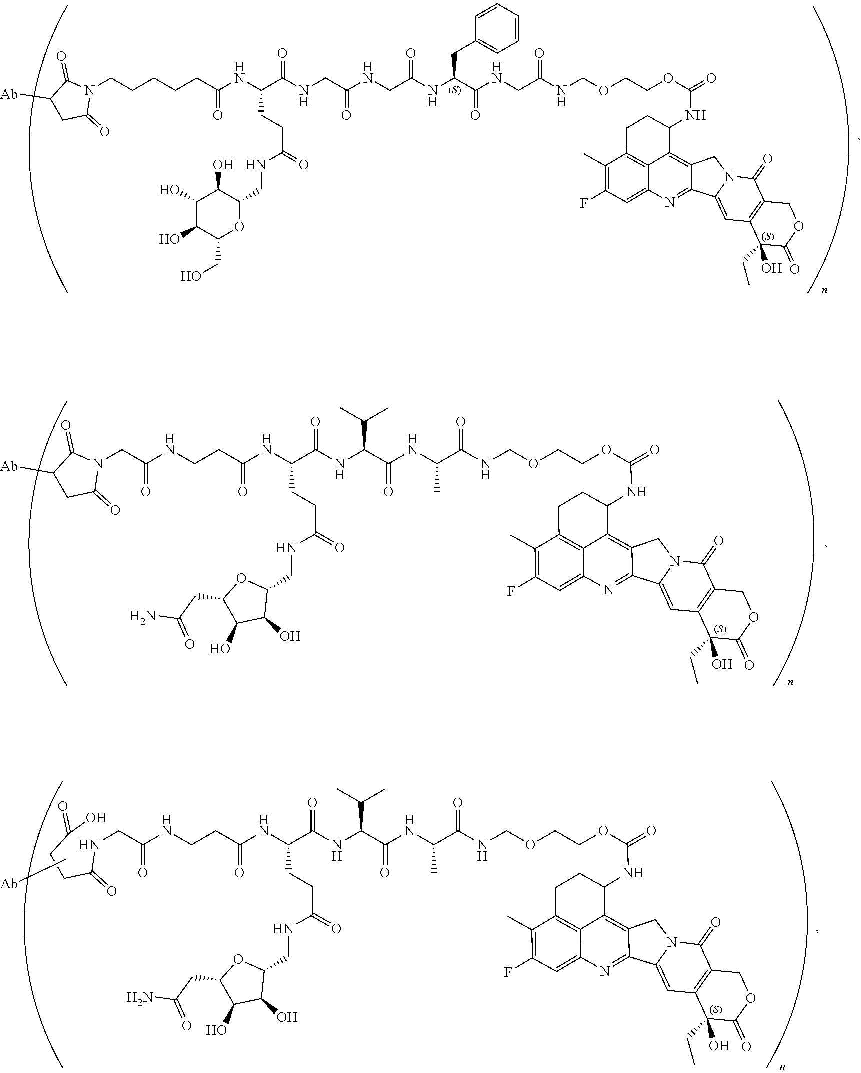

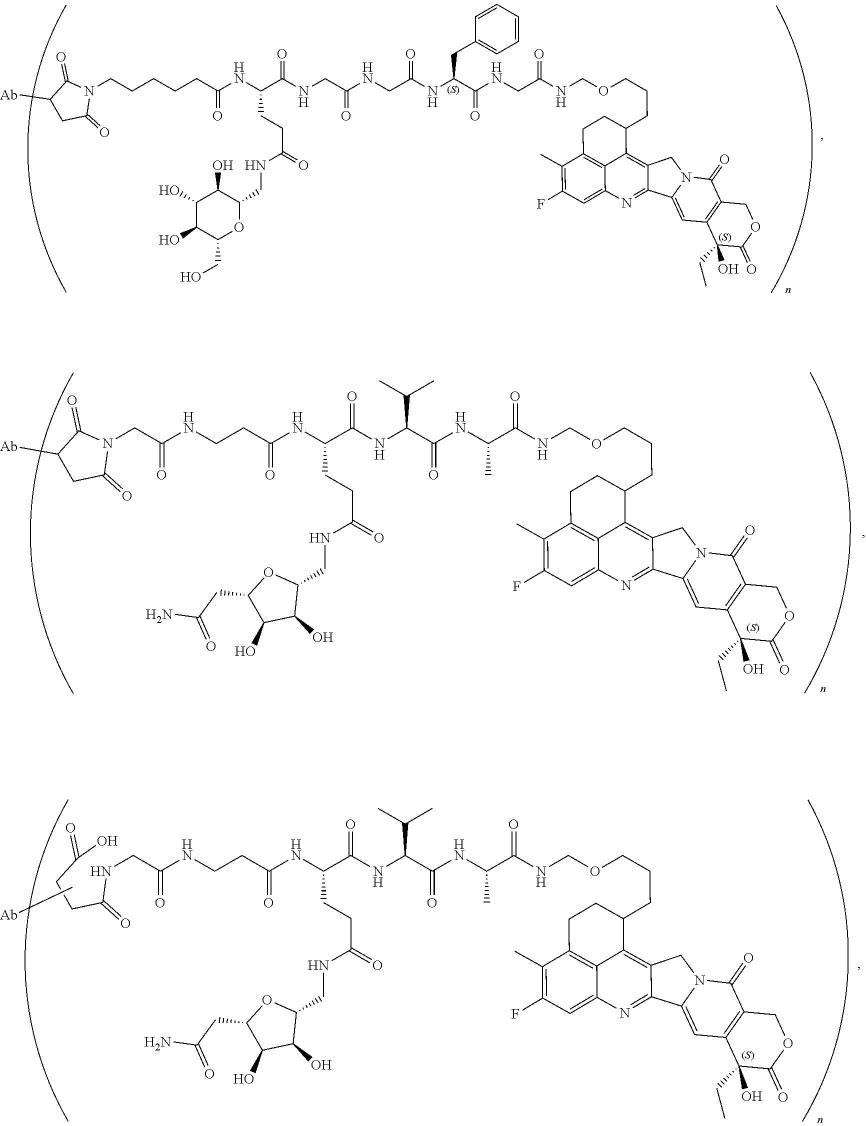

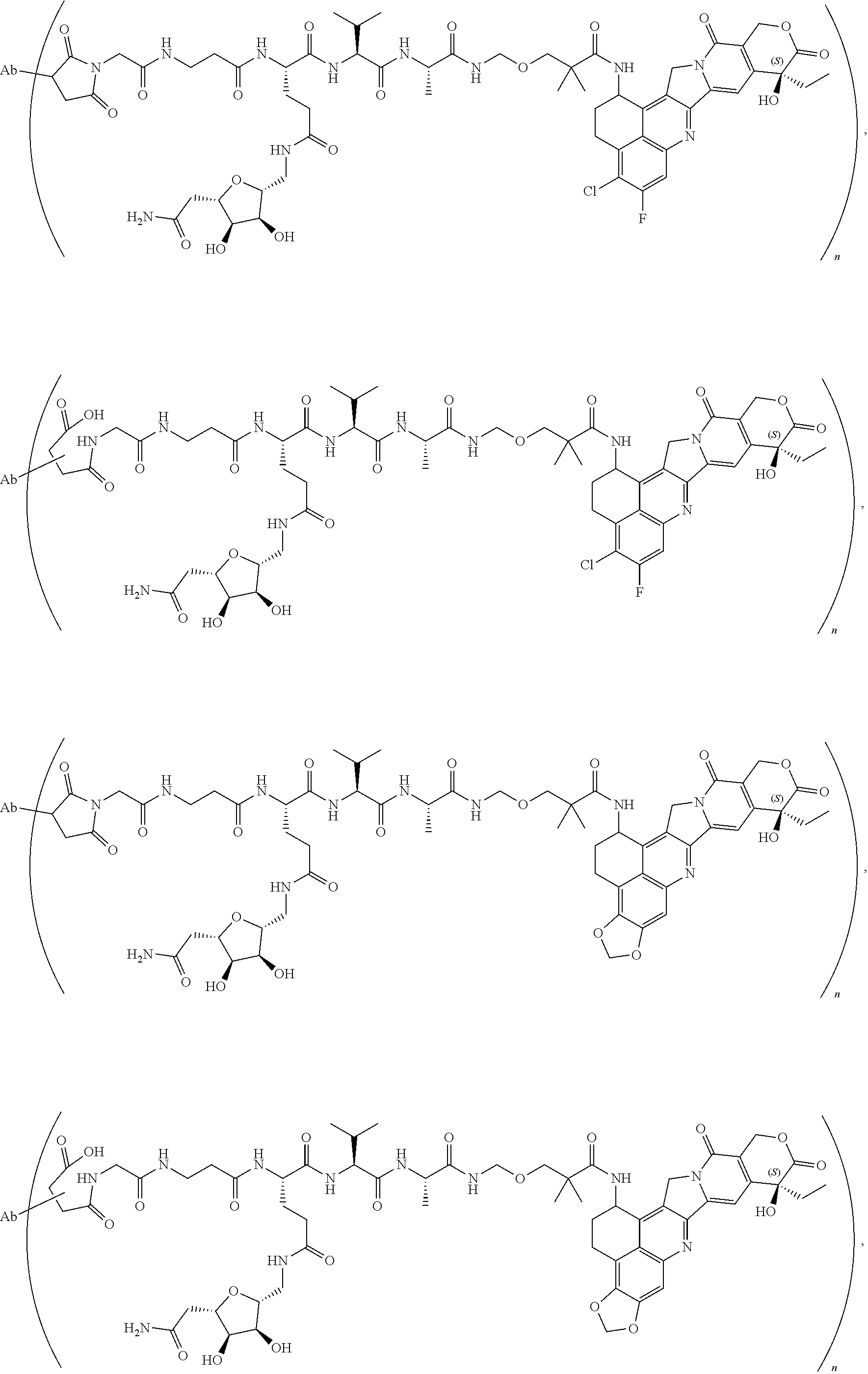

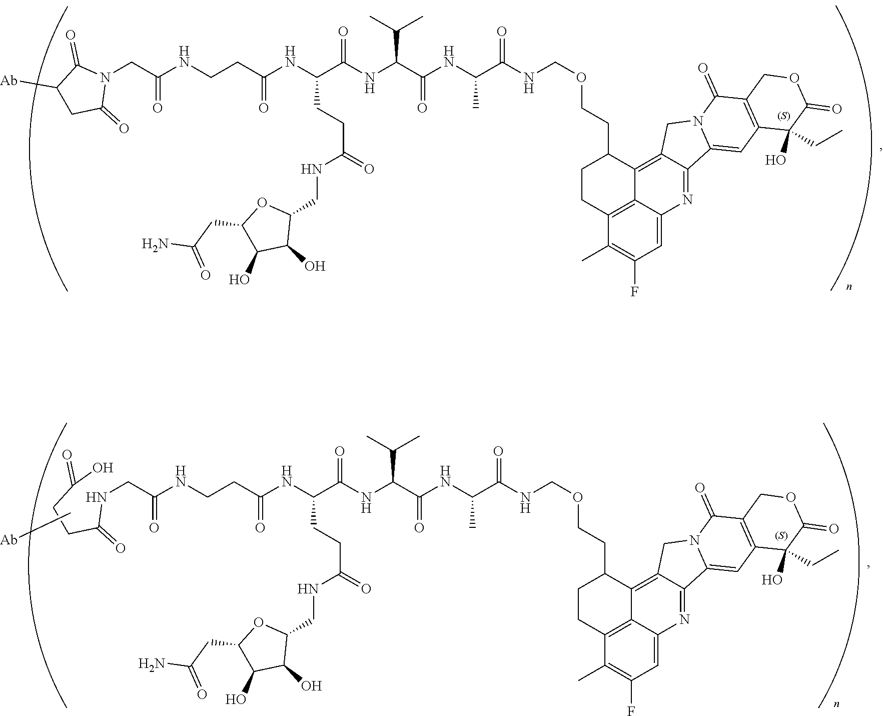

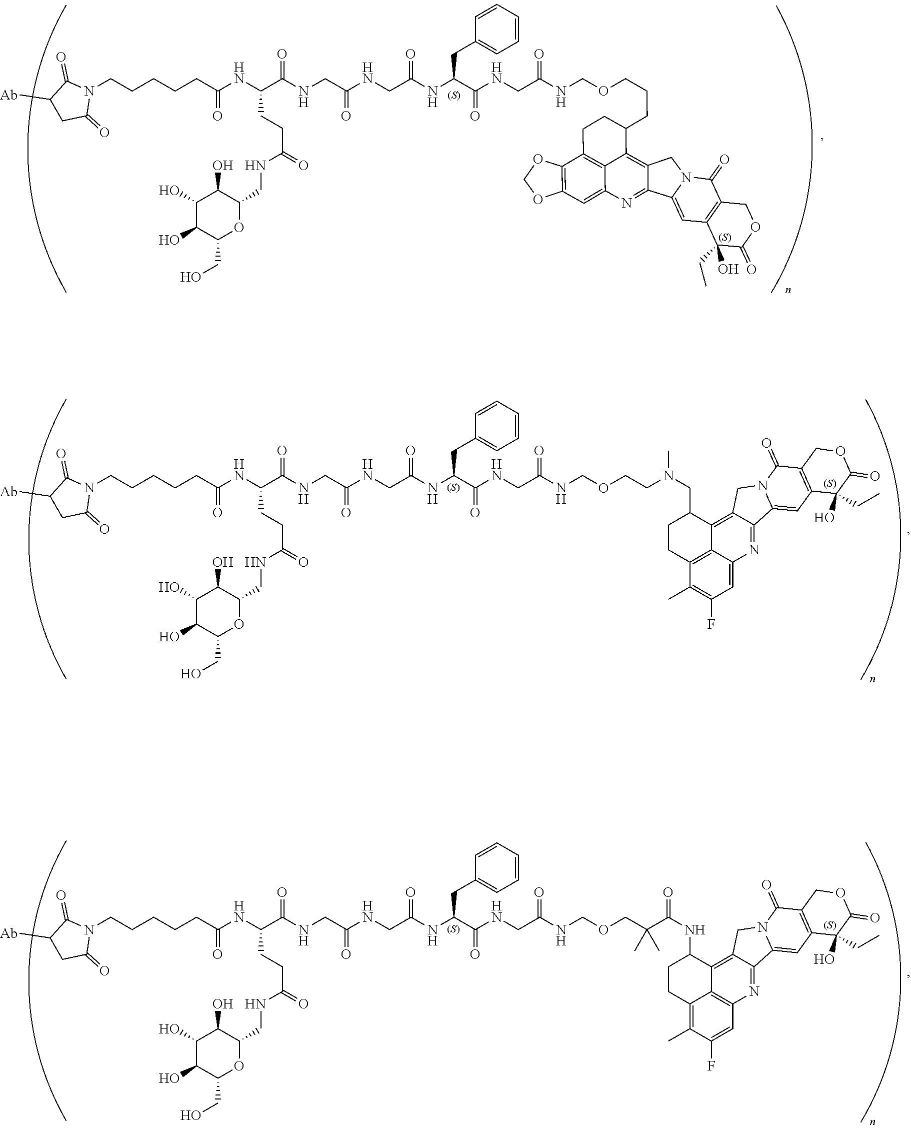

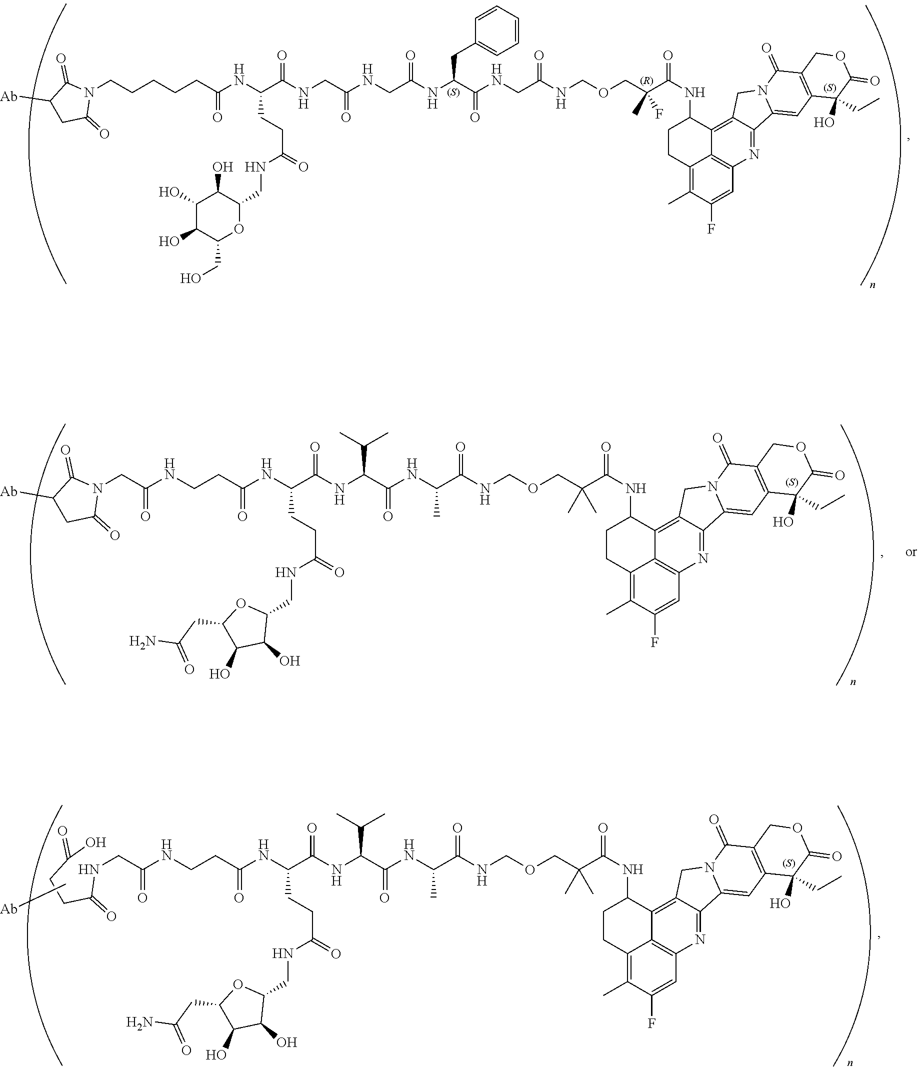

wherein Ab and n are as described herein.

In one embodiment, an anti-B3H3 antibody drug conjugate has one of the following structures:

| ADC | Antibody | |
|---|---|---|
| No. | No. | |
| ADC3-1 | BGA-6938 | 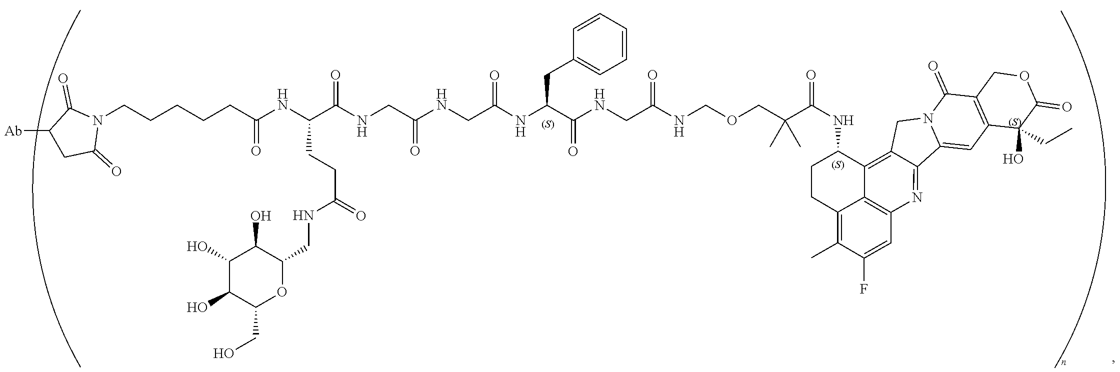 |
| ADC3-2 | BGA-6938 | 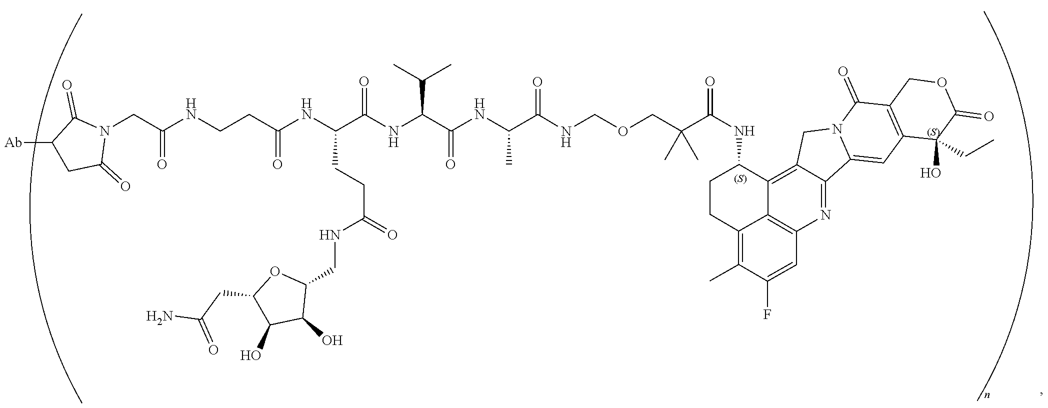 |
| | | 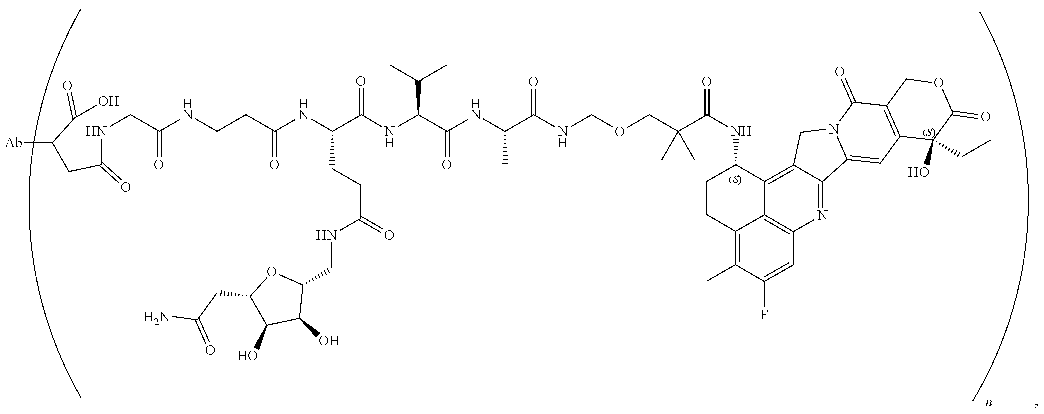 |
| ADC3-3 | BGA-6938 | 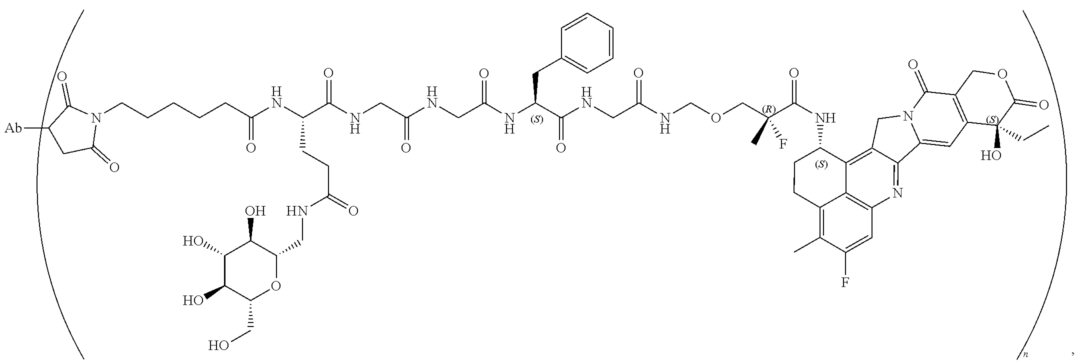 |
| ADC3-4 | BGA-6938 | 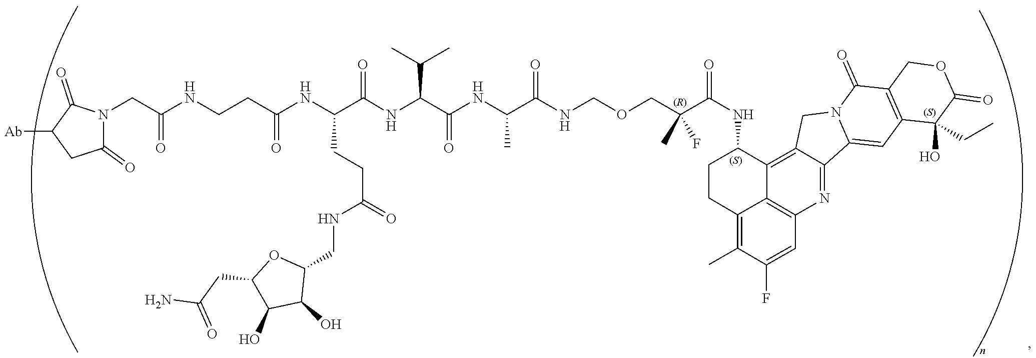 |
| | | 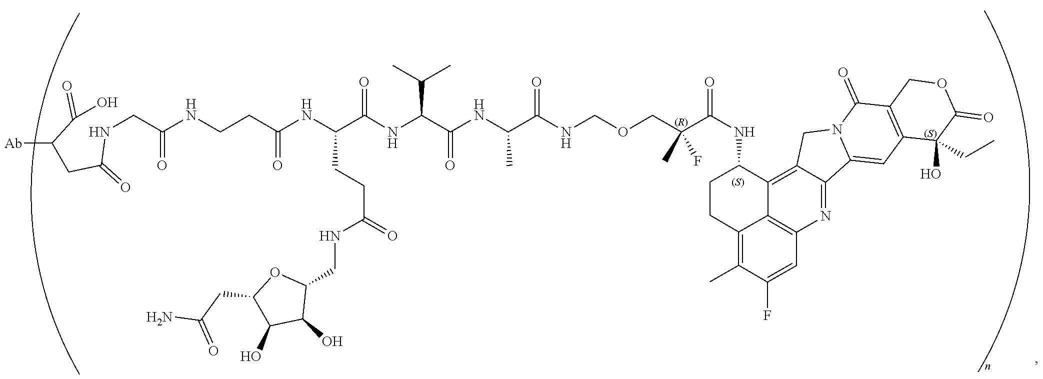 |
| ADC3-5 | BGA-6938 | 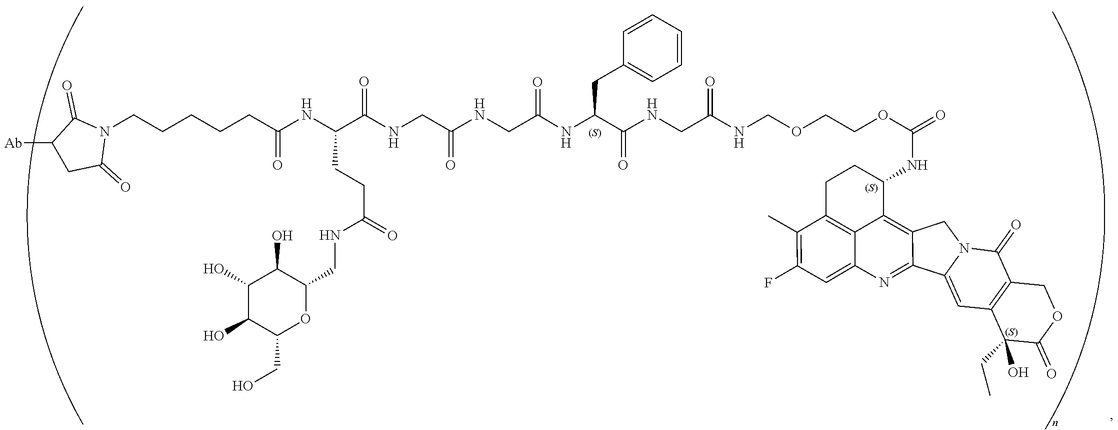 |
| ADC 3-6 | BGA-6938 | 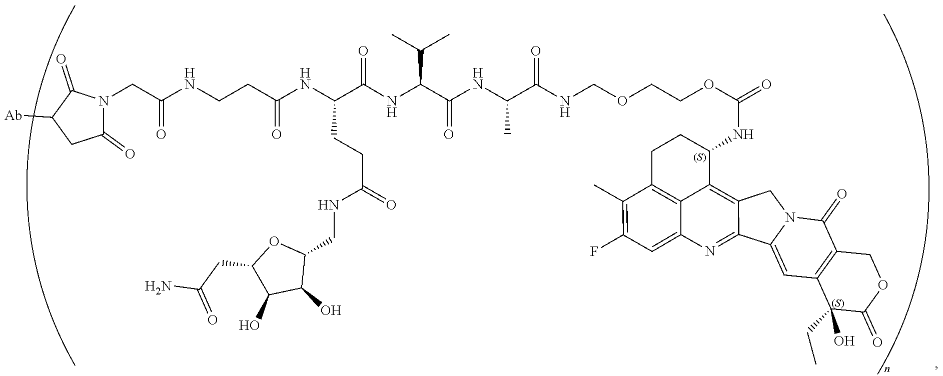 |
| | | 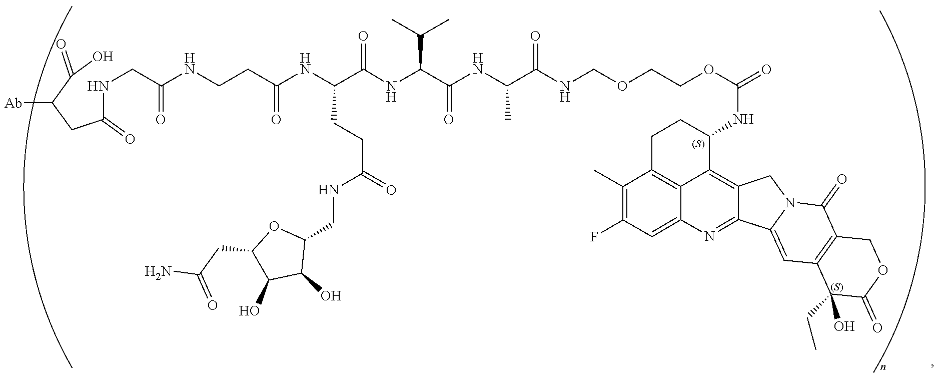 |
| ADC3-7 | BGA-6938 | 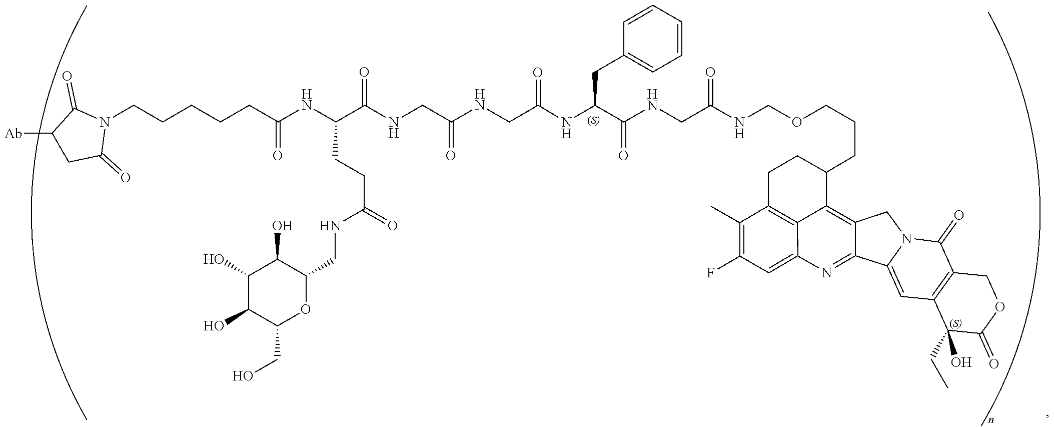 |
| ADC3-8 | BGA-6938 | 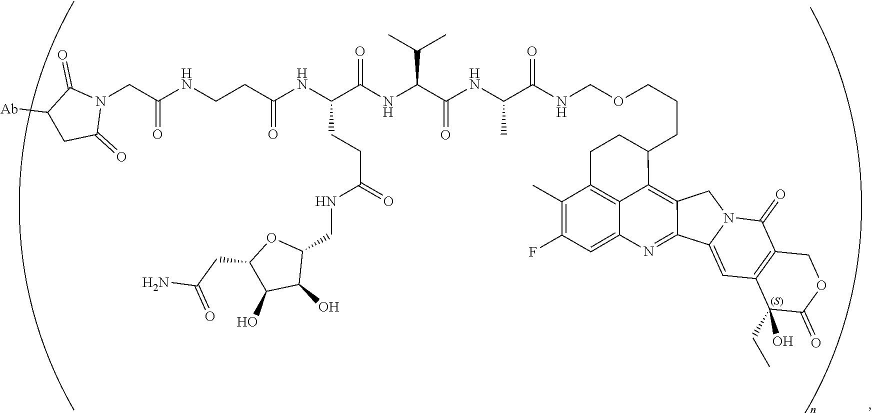 |
| | | 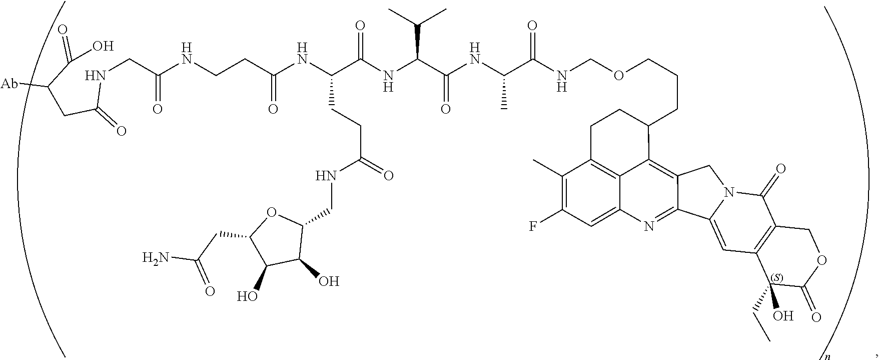 |
| ADC3-9 | BGA-6938 | 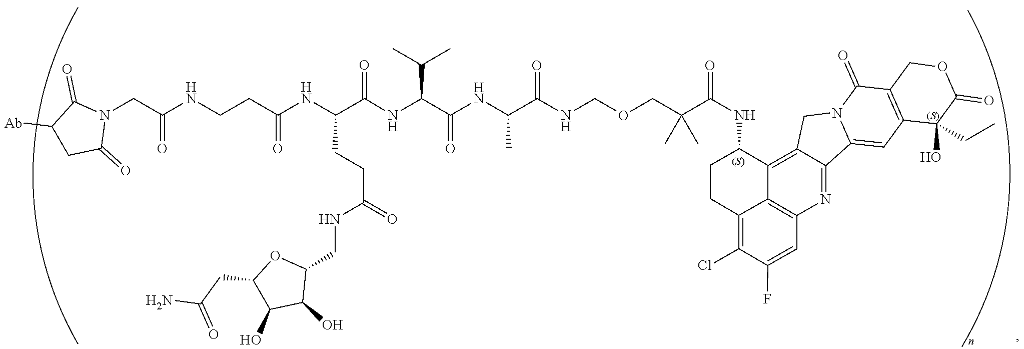 |
| | | 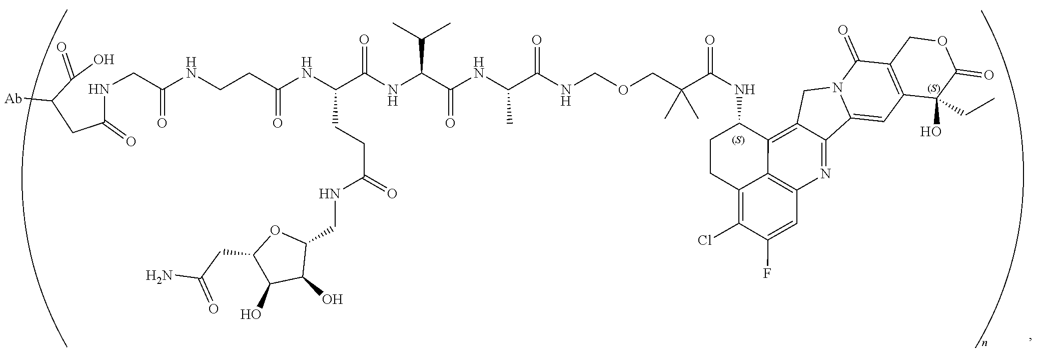 |
| ADC3- 10 | BGA-6938 | 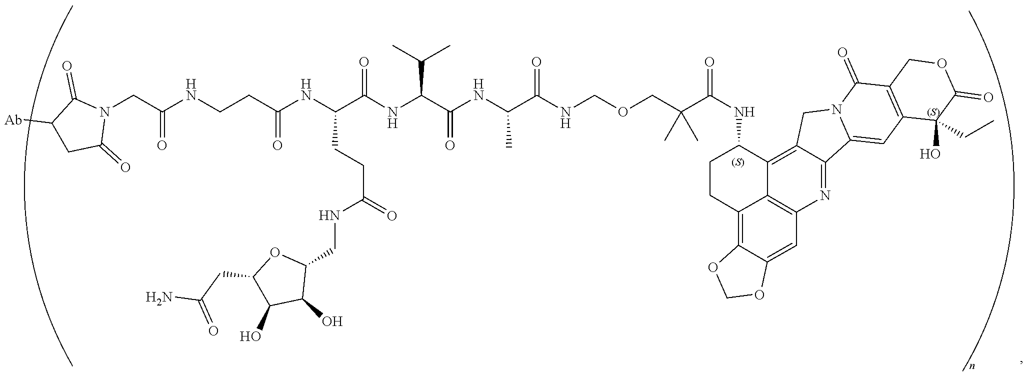 |
| | | 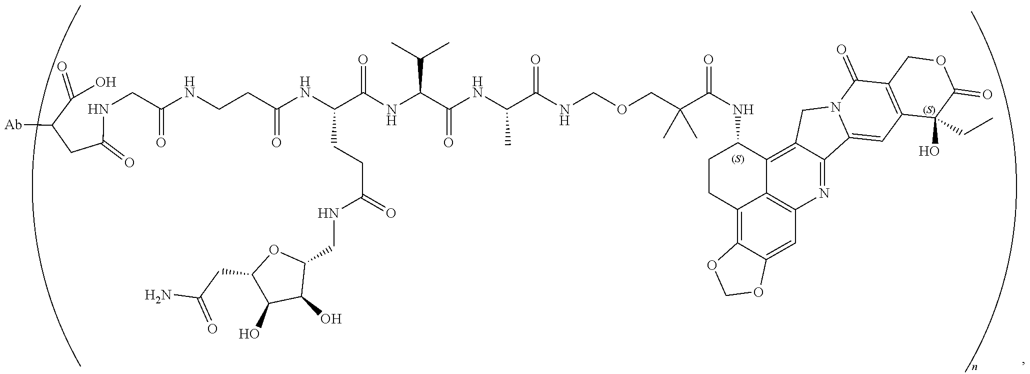 |
| ADC3- 11 | BGA-6938 | 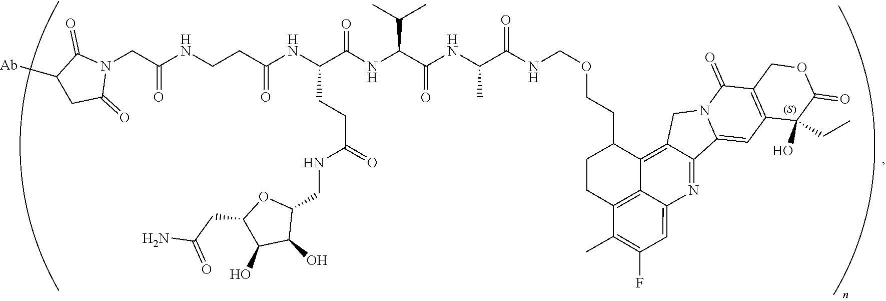 |
| | | 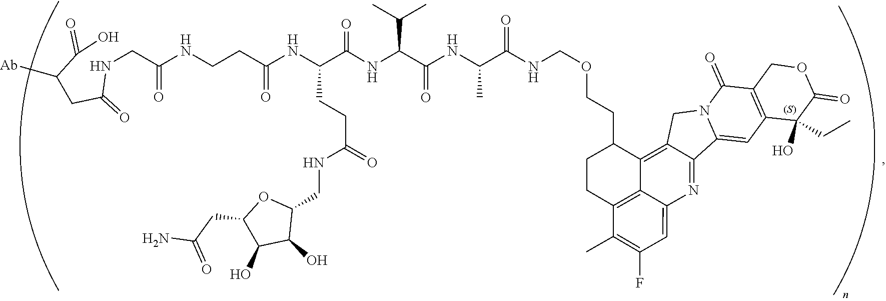 |
| ADC3- 12 | BGA-6938 | 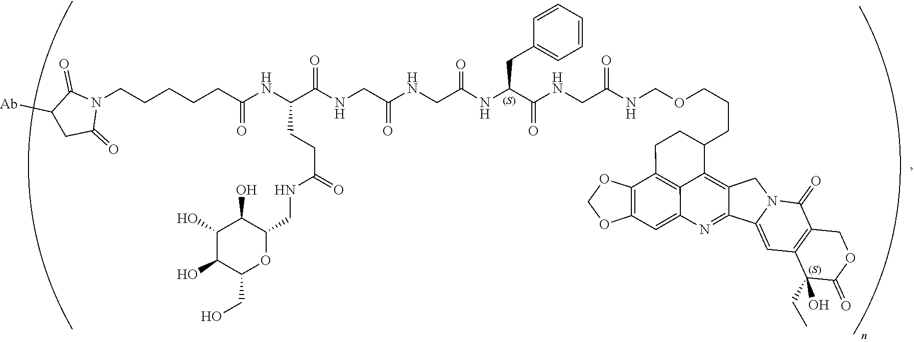 |
| ADC3- 13 | BGA-6938 | 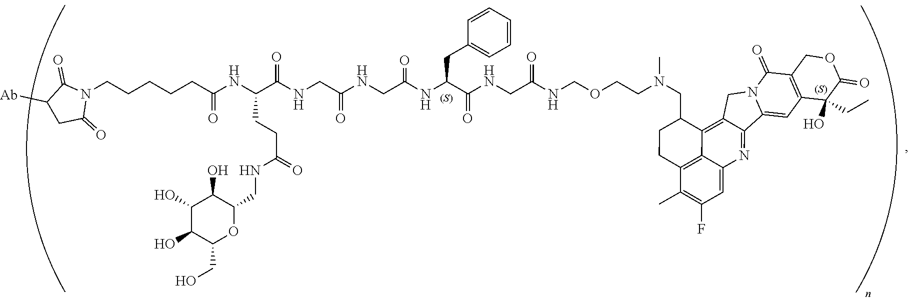 |
| ADC3- 14 | BGA-5063 | 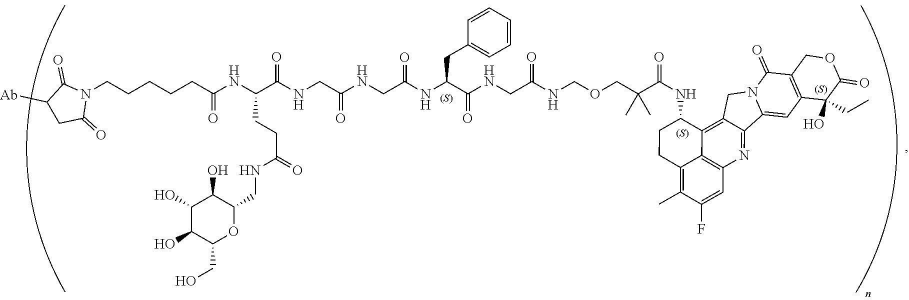 |
| ADC3- 15 | BGA-5063 | 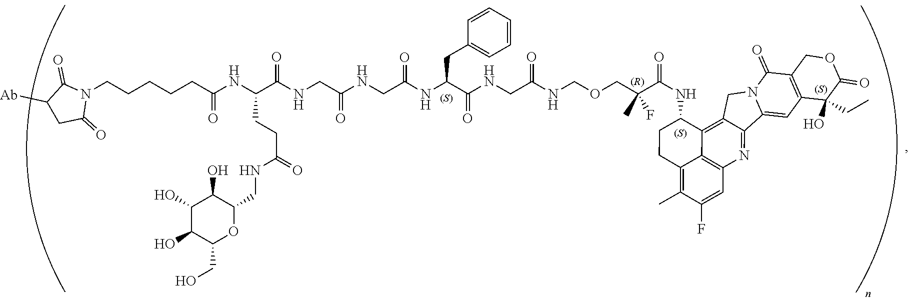 |
| ADC3- 16 | BGA-5063 | 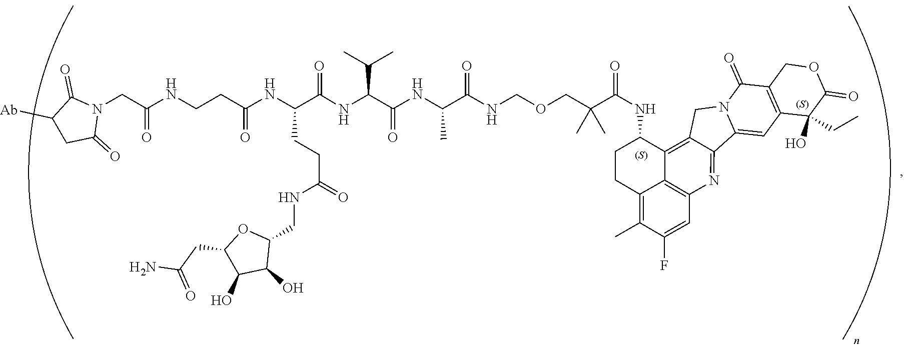 |
| | | or |
| | | 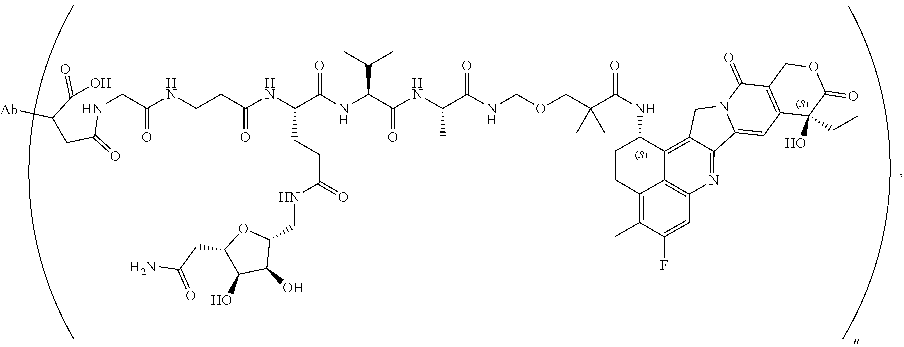 |

wherein n is as described herein.

In one embodiment, n is 4, 5, 6, 7, 8, 9, or 10. In one embodiment, n is S.

In one embodiment, provided here is a pharmaceutical composition comprising the antibody drug conjugate provided herein, and a pharmaceutically acceptable carrier.

### 7.2.3. Aspect 3

Provided here is an antibody or antigen binding fragment (Ab) thereof that binds B7H3, wherein the antibody or antigen-binding fragment comprises:
(i) a heavy chain variable region (VH) that comprises (a) a HCDR1 (Heavy Chain Complementarity Determining Region 1) of SEQ ID NO: 11, (b) a HCDR2 of SEQ ID NO: 2 and (c) a HCDR3 of SEQ ID NO: 14 and a light chain variable region (VL) that comprises: (d) a LCDR1 (Light Chain Complementarity Determining Region 1) of SEQ ID NO: 23, (e) a LCDRP2 of SEQ ID NO: 5, and (f) a LCDR3 of SEQ ID NO: 6;
(ii) a heavy chain variable region (VI) that comprises (a) a HCDR1 of SEQ ID NO: 1, (b) a HCDR2 of SEQ ID NO: 2 and (c) a HCDR3 of SEQ ID NO: 3 and a light chain variable region (VL) that comprises: (d) a LCDR1 of SEQ ID NO: 4, (e) a LCDR2 of SEQ ID NO: 5, and (f) a LCDR3 of SEQ ID NO: 6;
(iii) a heavy chain variable region (VH) that comprises (a) a HCDR1 of SEQ ID NO: 11, (b) a HCDR2 of SEQ ID NO: 2 and (c) a HCDR3 of SEQ ID NO: 3 and a light chain variable region (VL) that comprises: (d) a LCDR1 of SEQ ID NO: 4, (e) a LCDR2 of SEQ ID NO: 5, and (f) a LCDR3 of SEQ ID NO: 6;
(iv) a heavy chain variable region (VII) that comprises (a) a HCDR1 of SEQ ID NO: 1, (b) a HCDR2 of SEQ ID NO: 2 and (c) a HCDR3 of SEQ ID NO: 14; and a light chain variable region (VL) that comprises: (d) a LCDR1 of SEQ ID NO: 4, (e) a LCDR2 of SEQ ID NO: 5, and (f) a LCDR3 of SEQ ID NO: 6;
(v) a heavy chain variable region (VH) that comprises (a) a HCDR1 of SEQ ID NO: 1, (b) a HCDR2 of SEQ ID NO: 2 and (c) a HCDR3 of SEQ ID NO: 17; and a light chain variable region (VL) that comprises: (d) a LCDR1 of SEQ ID NO: 4, (e) a LCDR2 of SEQ ID NO: 5, and (f) a LCDR3 of SEQ ID NO: 6;
(vi) a heavy chain variable region (VH) that comprises (a) a HCDR1 of SEQ ID NO: 1, (b) a HCDR2 of SEQ ID NO: 2 and (c) a HCDR3 of SEQ ID NO: 3; and a light chain variable region (VL) that comprises: (d) a LCDR1 of SEQ ID NO: 20, (e) a LCDR2 of SEQ ID NO: 5, and (f) a LCDR3 of SEQ ID NO: 6;
(vii) a heavy chain variable region (VH) that comprises (a) a HCDR1 of SEQ ID NO: 1, (b) a HCDR2 of SEQ ID NO: 2 and (c) a HCDR3 of SEQ ID NO: 3; and a light chain variable region (VL) that comprises: (d) a LCDR1 of SEQ ID NO: 23, (e) a LCDR2 of SEQ ID NO: 5, and (f) a LCDR3 of SEQ ID NO: 6;
(viii) a heavy chain variable region (VH) that comprises (a) a HCDR1 of SEQ ID NO: 11, (b) a HCDR2 of SEQ ID NO: 2 and (c) a HCDR3 of SEQ ID NO: 28; and a light chain variable region (VL) that comprises: (d) a LCDR1 of SEQ ID NO: 23, (e) a LCDR2 of SEQ ID NO: 5, and (f) a LCDR3 of SEQ ID NO: 6; or (IX) a heavy chain variable region (VH) that comprises (a) a HCDR1 of SEQ ID NO: 300, (b) a HCDR2 of SEQ ID NO: 1700, and (c) a HCDR3 of SEQ ID NO: 500; and a light chain variable region (VL) that comprises (d) a LCDR1 of SEQ ID NO: 600, (e) a LCDR2 of SEQ ID NO: 700, and (f) a LCDR3 of SEQ ID NO: 800.

In one embodiment, the antibody or antigen-binding fragment comprises: (i) a heavy chain variable region comprising an amino acid sequence at least 90, 91, 92, 93, 94, 95, 96, 97, 98 or 99% identical to SEQ ID NO: 26, and a light chain variable region comprising an amino acid sequence at least 90, 91, 92, 93, 94, 95, 96, 97, 98 or 99% identical to SEQ ID NO: 24;
(ii) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 8;
(iii) a heavy chain variable region comprising an amino acid sequence at least 90, 91, 92, 93, 94, 95, 96, 97, 98 or 99% identical to SEQ ID NO: 12, and a light chain variable region comprising an amino acid sequence at least 90, 91, 92, 93, 94, 95, 96, 97, 98 or 99% identical to SEQ ID NO: 8;
(iv) a heavy chain variable region comprising an amino acid sequence at least 90, 91, 92, 93, 94, 95, 96, 97, 98 or 99% identical to SEQ ID NO: 15, and a light chain variable region comprising an amino acid sequence at least 90, 91, 92, 93, 94, 95, 96, 97, 98 or 99% identical to SEQ ID NO: 8;
(v) a heavy chain variable region comprising an amino acid sequence at least 90, 91, 92, 93, 94, 95, 96, 97, 98 or 99% identical to SEQ ID NO: 18, and a light chain variable region comprising an amino acid sequence at least 90, 91, 92, 93, 94, 95, 96, 97, 98 or 99% identical to SEQ ID NO: 8;
(vi) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 21;
(vii) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 24;
(viii) a heavy chain variable region comprising an amino acid sequence at least 90, 91, 92, 93, 94, 95, 96, 97, 98 or 99% identical to SEQ ID NO: 29, and a light chain variable region comprising an amino acid sequence at least 90, 91, 92, 93, 94, 95, 96, 97, 98 or 99% identical to SEQ ID NO: 24; or
(ix) a heavy chain variable region comprising an amino acid sequence at least 90, 91, 92, 93, 94, 95, 96, 97, 98 or 99% identical to SEQ ID NO: 1800, and a light chain variable region comprising an amino acid sequence at least 90, 91, 92, 93, 94, 95, 96, 97, 98 or 99% identical to SEQ ID NO: 1400.

In one embodiment, one, two, three, four, five, six, seven, eight, nine, or ten amino acids within each of SEQ ID NOs: 26 and 24, each of SEQ ID NOs: 7 and 8, each of SEQ ID NOs: 12 and 8, each of SEQ ID NOs: 15 and 8, each of SEQ ID NOs: 18 and 8, each of SEQ ID NOs: 7 and 21, each of SEQ ID Nos: 7 and 24, each of SEQ ID Nos: 29 and 24, or each of SEQ ID NOs: 1800 and 1400, have been inserted, deleted or substituted in the antibody or antigen-binding fragment.

In one embodiment, the antibody or antigen-binding fragment comprises: (i) a heavy chain variable region comprising SEQ ID NO: 26, and a light chain variable region comprising SEQ ID NO: 24;
(ii) a heavy chain variable region comprising SEQ ID NO: 7, and a light chain variable region comprising SEQ ID NO: 8;
(iii) a heavy chain variable region comprising SEQ ID NO: 12, and a light chain variable region comprising SEQ ID NO: 8;
(iv) a heavy chain variable region comprising SEQ ID NO: 15, and a light chain variable region comprising SEQ ID NO: 8;
(v) a heavy chain variable region comprising SEQ ID NO: 18, and a light chain variable region comprising SEQ ID NO: 8;
(vi) a heavy chain variable region comprising SEQ ID NO: 7, and a light chain variable region comprising SEQ ID NO: 21;
(vii) a heavy chain variable region comprising SEQ ID NO: 7, and a light chain variable region comprising SEQ ID NO: 24;
(viii) a heavy chain variable region comprising SEQ ID NO: 29, and a light chain variable region comprising SEQ ID NO: 24; or
(ix) a heavy chain variable region comprising SEQ ID NO: 1800, and a light chain variable region comprising SEQ ID NO: 1400.

In one embodiment, the antibody or antigen-binding fragment is a monoclonal antibody, a human engineered antibody, a single chain antibody (scFv), a Fab fragment, a Fab′ fragment, or a F(ab′)₂ fragment.

In one embodiment, the antibody or antigen-binding fragment comprises a scFv comprising a VH having the amino acid of SEQ ID NO: 26 and a VL having an amino acid of SEQ ID NO: 24, optionally the VH and VL are connected via an amino acid linker, optionally the amino acid linker is any sequence of SEQ ID NO: 35 to SEQ ID NO: 77.

In one embodiment, the antibody or antigen-binding fragment comprises a scFv having the amino acid sequence of SEQ ID NO: 32.

In one embodiment, the antibody or antigen-binding fragment thereof comprises a heavy chain constant region of the subclass of IgG1, IgG2, IgG3, or IgG4, and/or a light chain constant region of the type of kappa or lambda.

In one embodiment, the antibody or antigen-binding fragment thereof comprises a heavy chain constant region of the subclass of IgG1, and a light chain constant region of the type of kappa.

### 7.2.4. Aspect 4

Provided here is an antibody drug conjugate, wherein the antibody drug conjugate is

| ADC No. | Antibody No. | |
|---|---|---|
| ADC3-1 | BGA-6938 | 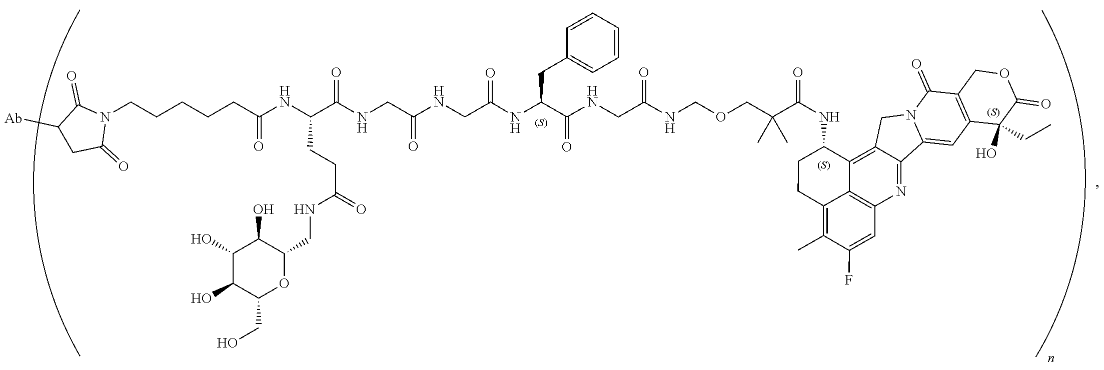 |
| ADC3-2 | BGA-6938 | 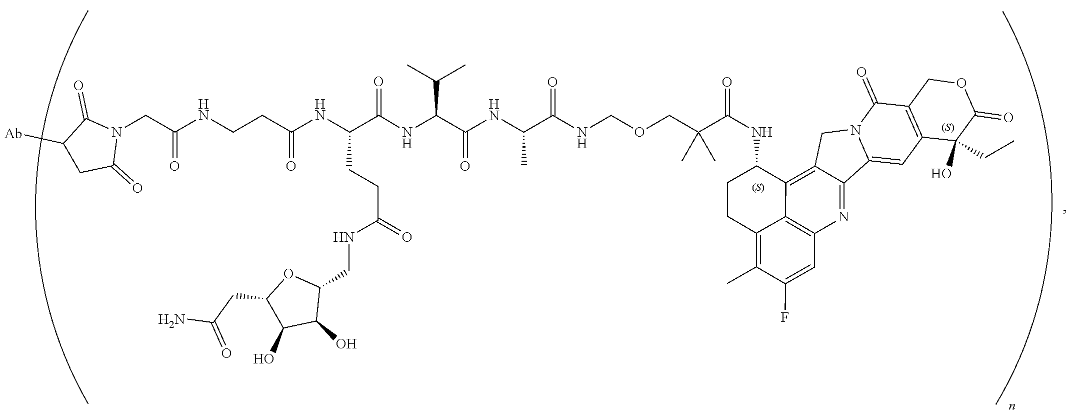 |
| | | 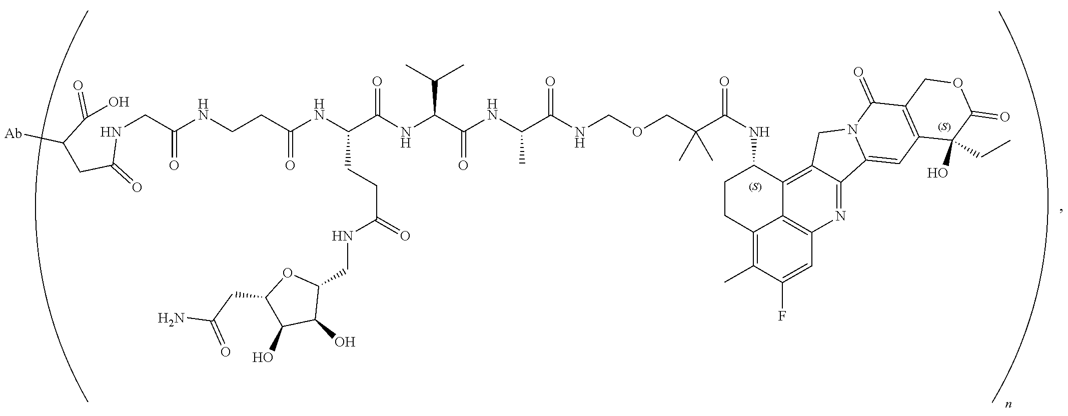 |
| ADC3-3 | BGA-6938 | 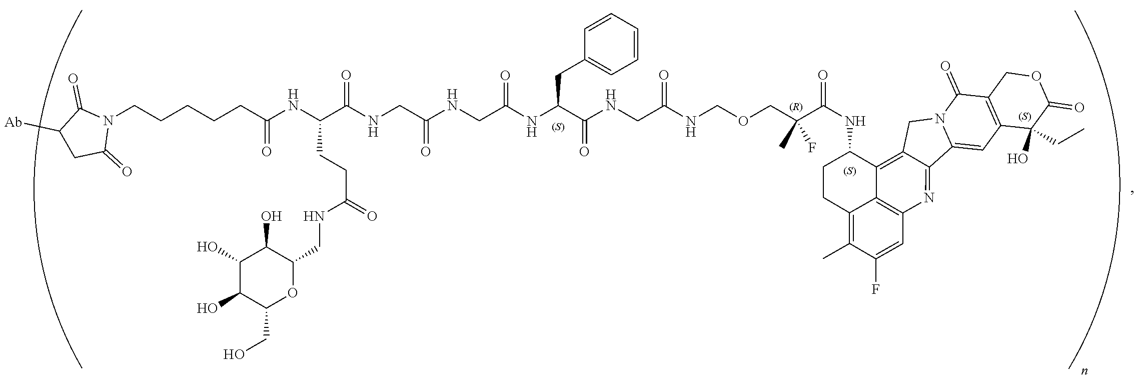 |
| ADC3-4 | BGA-6938 | 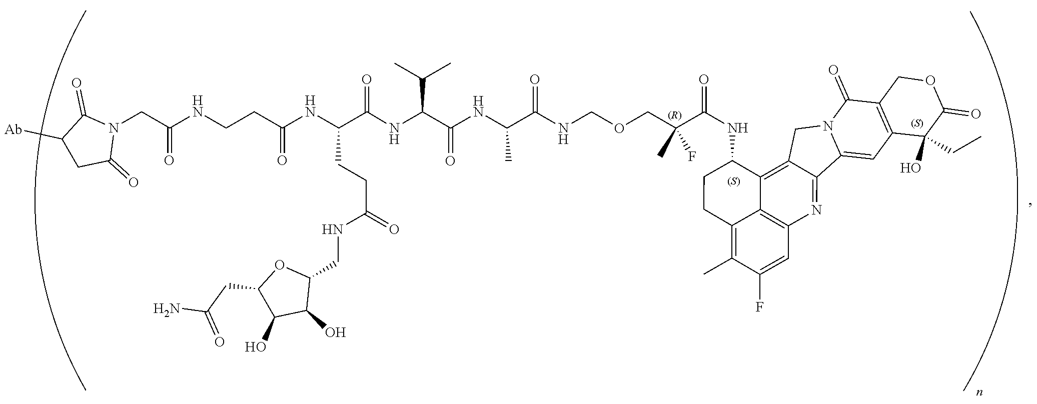 |
| | | 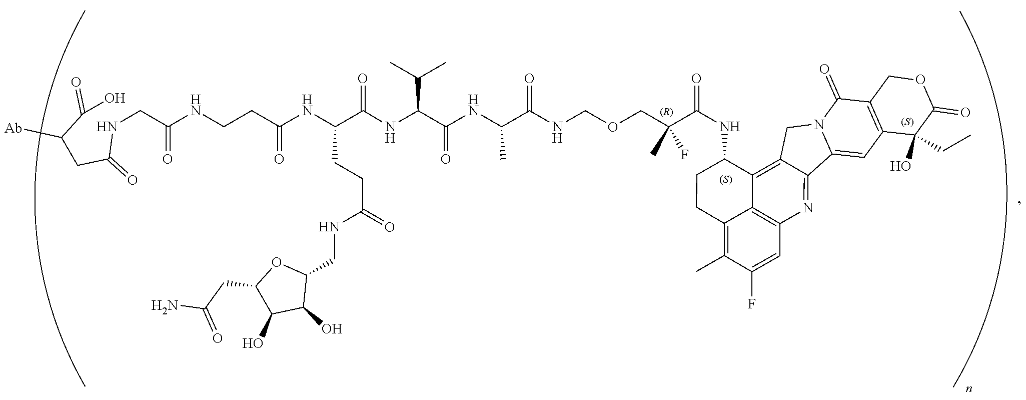 |
| ADC3-5 | BGA-6938 | 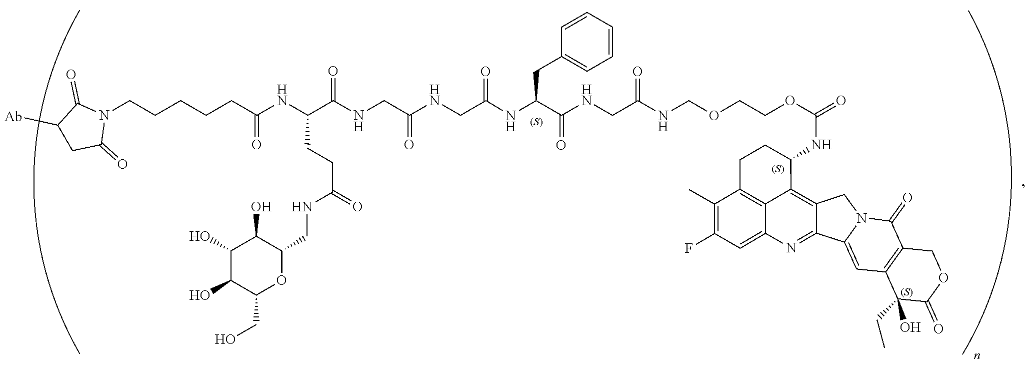 |
| ADC 3-6 | BGA-6938 | 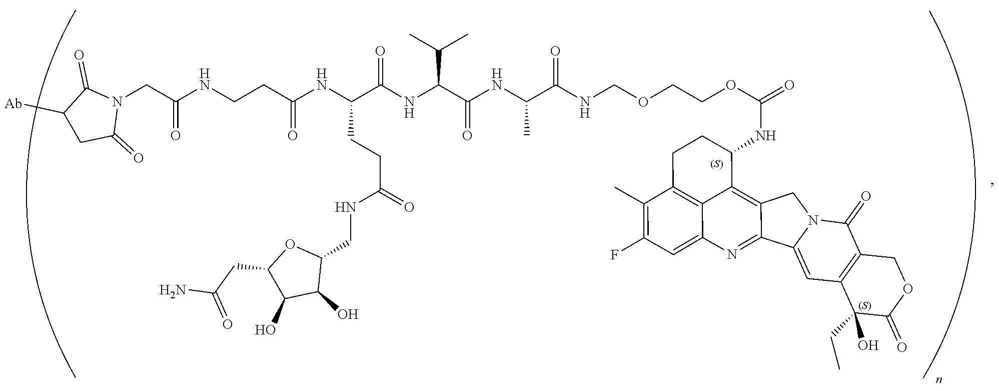 |
| | | 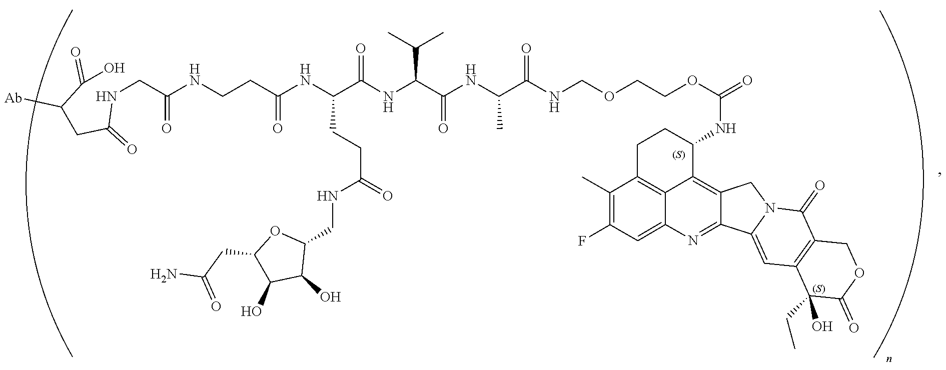 |
| ADC3-7 | BGA-6938 | 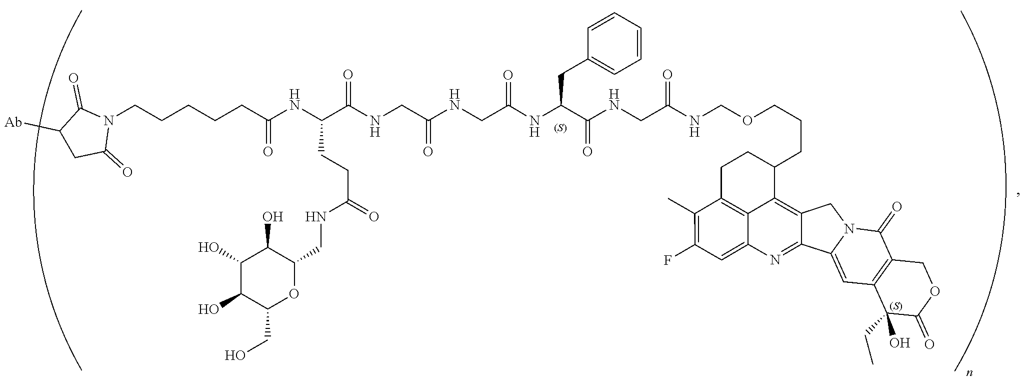 |
| ADC3-8 | BGA-6938 | 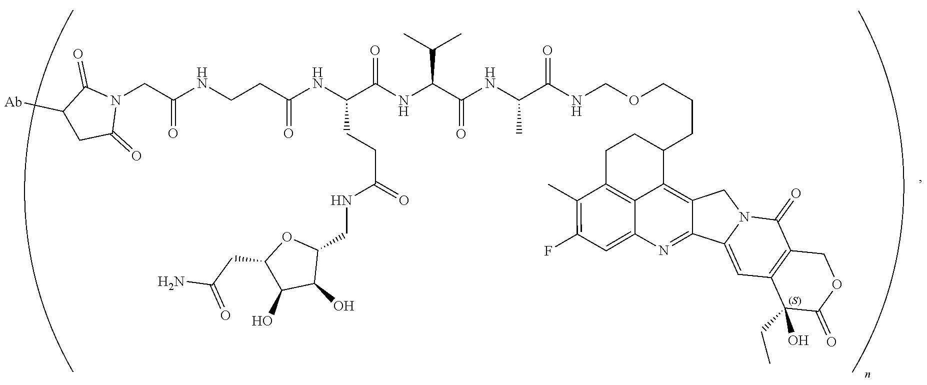 |
| | | 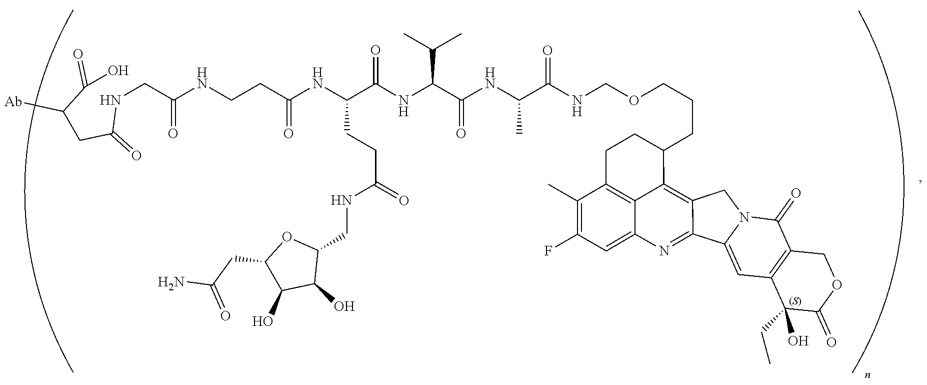 |
| ADC3-9 | BGA-6938 | 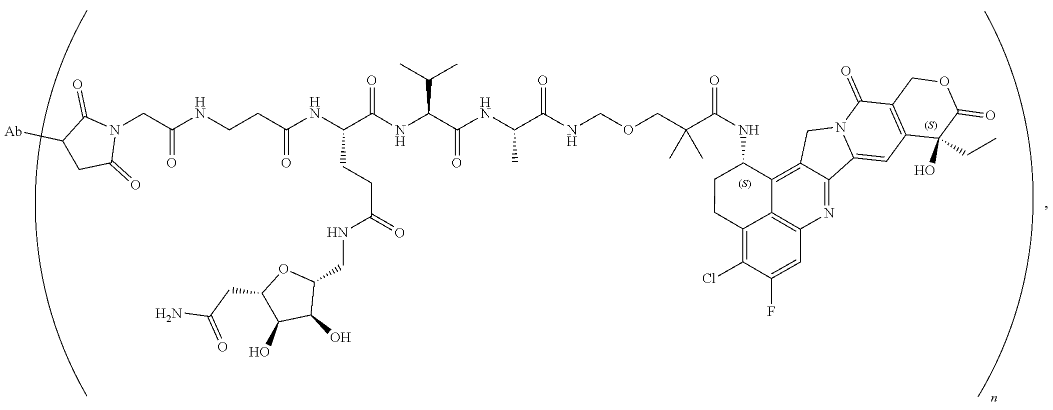 |
| | | 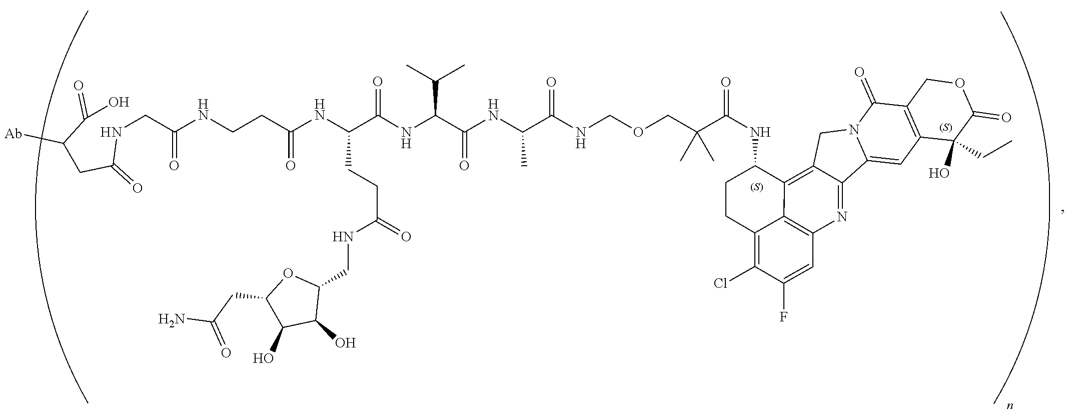 |
| ADC3-10 | BGA-6938 | 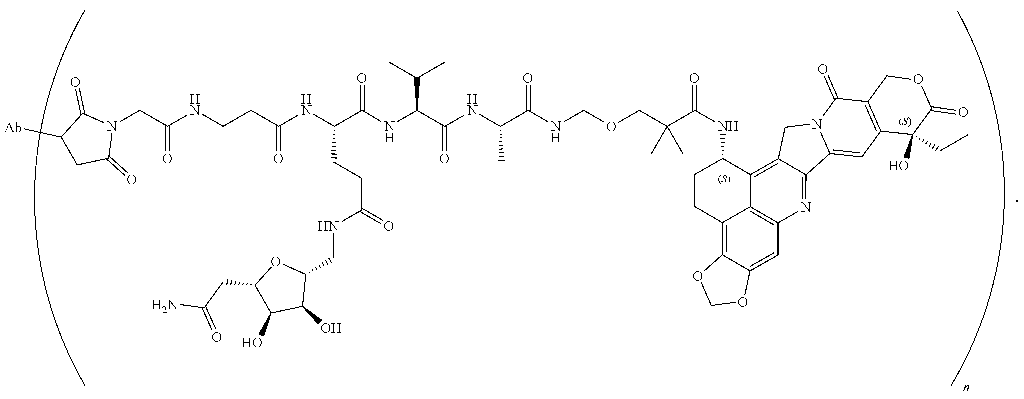 |
| | | 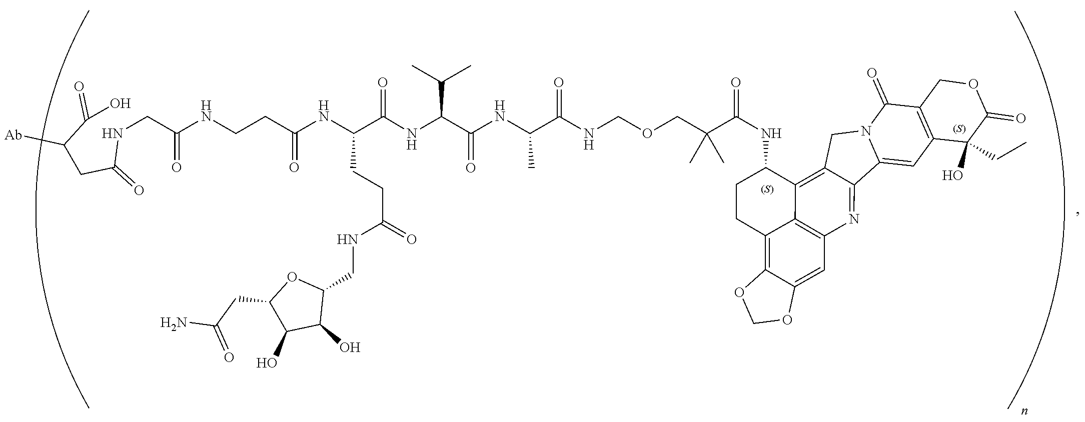 |
| ADC3-11 | BGA-6938 | 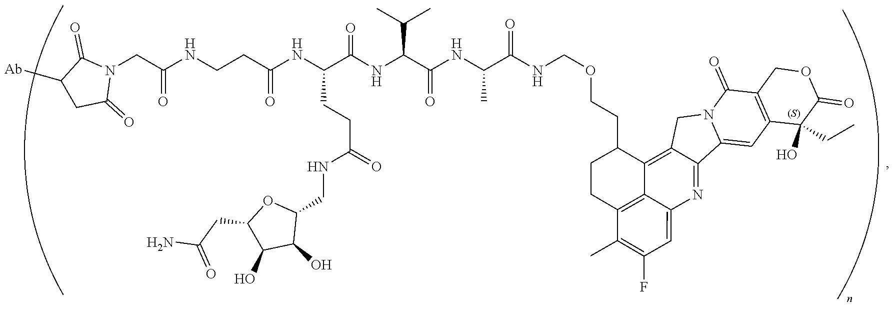 |
| | | 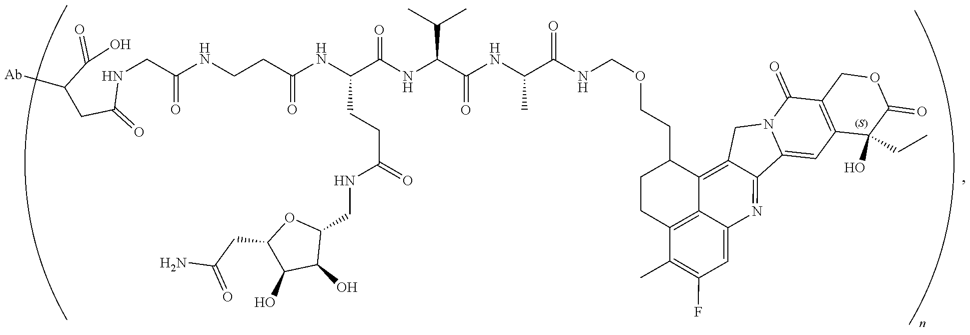 |
| ADC3-12 | BGA-6938 | 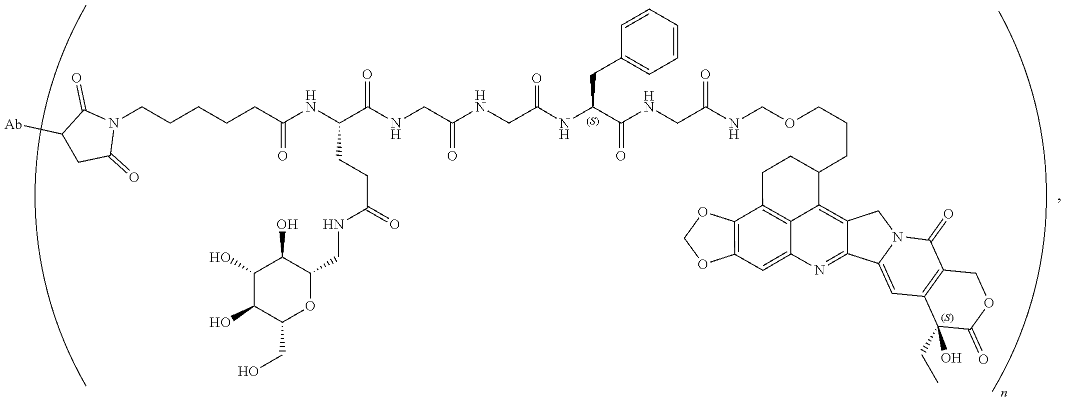 |
| ADC3-13 | BGA-6938 | 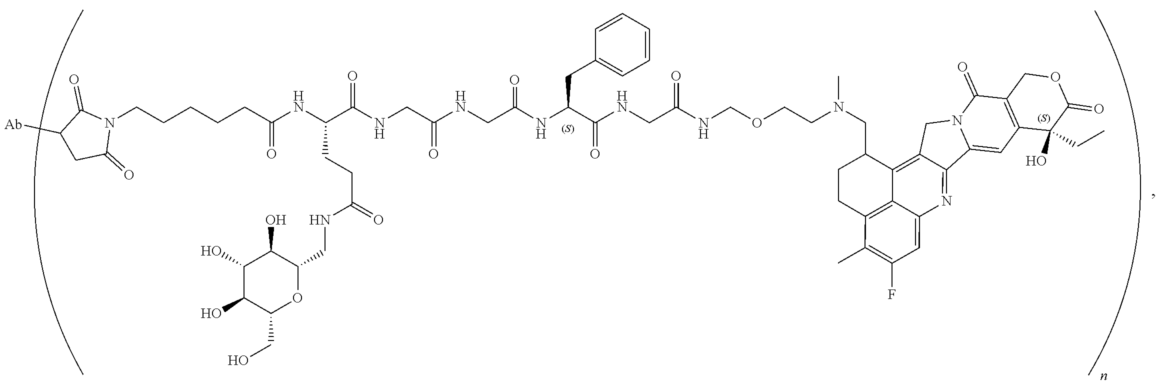 |
| ADC3-14 | BGA-5063 | 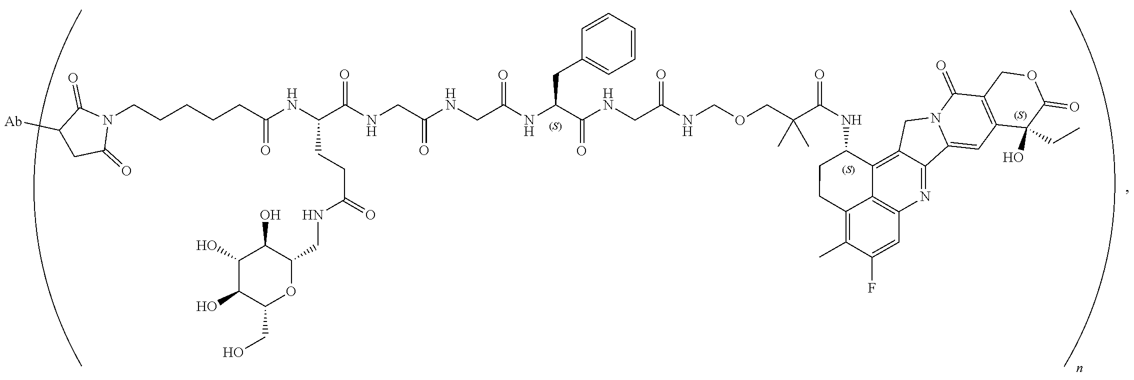 |
| ADC3-15 | BGA-5063 | 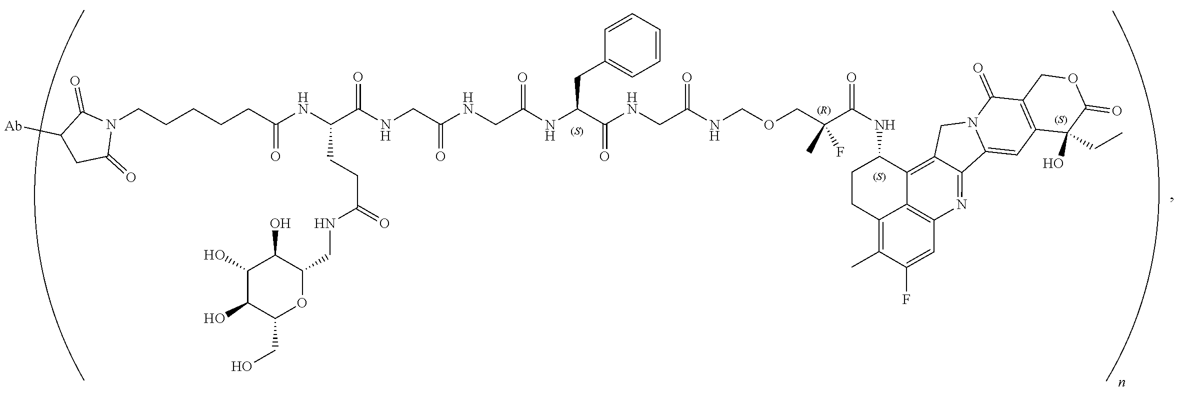 |
| ADC3-16 | BGA-5063 | 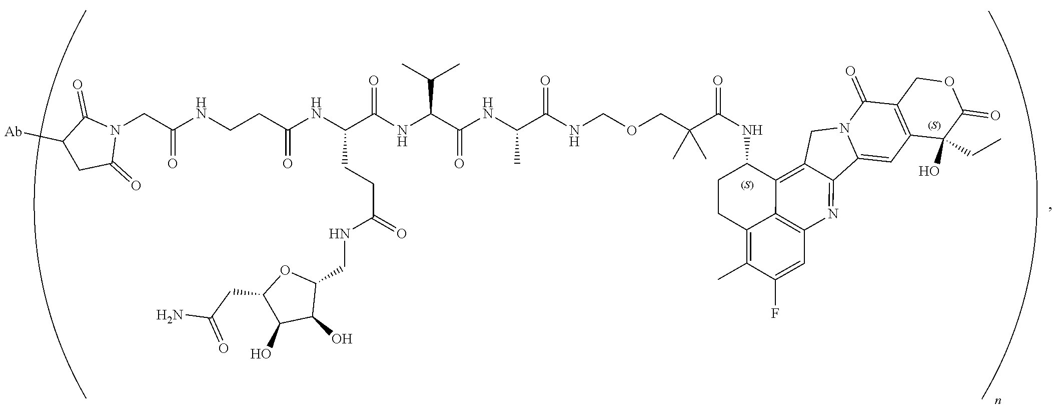 |
| | | or |
| | | 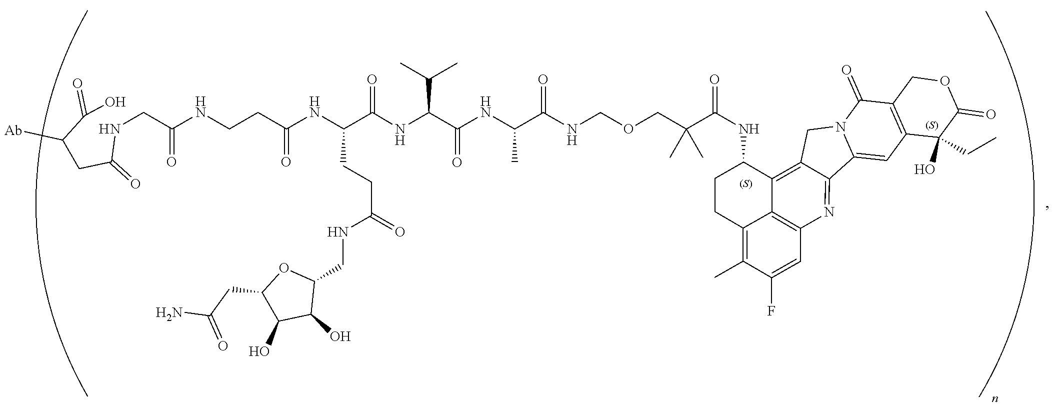 |

or a pharmaceutically acceptable salt thereof;
n is 4, 5, 6, 7, 8, 9, or 10; and
Ab is an antibody or antigen binding fragment thereof that binds B7H3; the antibody or antigen-binding fragment comprises:
(i) a heavy chain variable region (VH) that comprises (a) a HCDR1 (Heavy Chain Complementarity Determining Region 1) of SEQ ID NO: 11, (b) a HCDR2 of SEQ ID NO: 2, and (c) a HCDR3 of SEQ ID NO: 14; and a light chain variable region (VL) that comprises (d) a LCDR1 (Light Chain Complementarity Determining Region 1) of SEQ ID NO: 23, (e) a LCDR2 of SEQ ID NO: 5, and (f) a LCDR3 of SEQ ID NO: 6;
(ii) a heavy chain variable region (VH) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises (d) a LCDR1 of SEQ ID NO: 4, (e) a LCDR2 of SEQ ID NO: 5, and (f) a LCDR3 of SEQ ID NO: 6;
(iii) a heavy chain variable region (VII) that comprises (a) a HCDR1 of SEQ ID NO: 11, (b) a HCDR2 of SEQ ID NO: 2, and (c) a HCDR3 of SEQ ID NO: 3; and a light chain variable region (VL) that comprises (d) a LCDR1 of SEQ ID NO: 4, (e) a LCDR2 of SEQ ID NO: 5, and (f) a LCDR3 of SEQ ID NO: 6;
(iv) a heavy chain variable region (VII) that comprises (a) a HCDR1 of SEQ ID NO: 1, (b) a HCDR2 of SEQ ID NO: 2, and (c) a HCDR3 of SEQ ID NO: 14; and a light chain variable region (VL) that comprises (d) a LCDR1 of SEQ ID NO: 4, (e) a LCDR2 of SEQ ID NO: 5, and (f) a LCDR3 of SEQ ID NO: 6;
(v) a heavy chain variable region (VI) that comprises (a) a HCDR1 of SEQ ID NO: 1, (b) a HCDR2 of SEQ ID NO: 2, and (c) a HCDR3 of SEQ ID NO: 17; and a light chain variable region (VL) that comprises (d) a LCDR1 of SEQ ID NO: 4, (e) a LCDR2 of SEQ ID NO: 5, and (f) a LCDR3 of SEQ ID NO: 6;
(vi) a heavy chain variable region (VH) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises (d) a LCDR1 of SEQ ID NO: 20, (e) a LCDR2 of SEQ ID NO: 5, and (f) a LCDR3 of SEQ ID NO: 6;
(vii) a heavy chain variable region (VII) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises: (d) a LCDR1 of SEQ ID NO: 23, (e) a LCDR2 of SEQ ID NO: 5, and (f) a LCDR3 of SEQ ID NO: 6;
(viii) a heavy chain variable region (VH) that comprises (a) a HCDR1 of SEQ ID NO: 11, (b) a HCDR2 of SEQ ID NO: 2, and (c) a HCDR3 of SEQ ID NO: 28; and a light chain variable region (VL) that comprises: (d) a LCDR1 of SEQ ID NO: 23, (e) a LCDR2 of SEQ ID NO: 5, and (f) a LCDR3 of SEQ ID NO: 6; or
(IX) a heavy chain variable region (VH) that comprises (a) a HCDR1 of SEQ ID NO: 300, (b) a HCDR2 of SEQ ID NO: 1700, and (c) a HCDR3 of SEQ ID NO: 500; and a light chain variable region (VL) that comprises (d) a LCDR1 of SEQ ID NO: 600, (e) a LCDR2 of SEQ ID NO: 700, and (f) a LCDR3 of SEQ ID NO: 800.

In one embodiment, n is 8.

Provided herein is a pharmaceutical composition comprising the antibody drug conjugate provided herein, and a pharmaceutically acceptable carrier.

### 7.3. Methods of Treatment

The antibody drug conjugates of the present disclosure are useful in a variety of applications including, but not limited to, methods for the treatment of a B7H3-associated disorder or disease. In one aspect, the B7H3-associated disorder or disease is cancer. In some embodiments, the cell is 4Ig-B7H3 positive.

Accordingly, provided herein is a method of treating a cancer comprising administering to a patient in need thereof an effective amount of the antibody drug conjugate provided herein, or a pharmaceutical composition provided herein. In some embodiments, the cancer is 4Ig-B7H3 positive. In some embodiments, the cancer is colorectal carcinoma, prostate cancer, pancreatic cancer, breast cancer, ovarian cancer, kidney cancer, lung cancer, or esophageal carcinoma. In some embodiments, the cancer is lung cancer, e.g., non-small cell lung cancer (NSCLC) or small cell lung cancer (SCLC). In some embodiments, the cancer is squamous non-small cell lung cancer. In some embodiments, the cancer is esophageal carcinoma, e.g., esophageal squamous cell carcinoma.

The antibody drug conjugates disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibody drug conjugates of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody drug conjugate need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

### 7.4. Combination Therapy

The antibody drug conjugates described herein are, in some embodiments, administered in combination with another therapeutic agent. Other therapeutic agents that can be used with the antibody drug conjugates of the present disclosure include, but are not limited to, a chemotherapeutic agent (e.g., paclitaxel or a paclitaxel agent (e.g. Abraxane®), docetaxel, carboplatin, topotecan, cisplatin, irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor) (e.g., erlotinib), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, or Polo-like kinase 1 inhibitor (e.g., BI 672).

In some embodiments, the therapeutic agent is paclitaxel or a paclitaxel agent, docetaxel, carboplatin, topotecan, cisplatin, irinotecan, doxorubicin, lenalidomide, or 5-azacytidine.

In some embodiments, the therapeutic agent is an immune checkpoint inhibitor. In some embodiments, the immune checkpoint inhibitor is an anti-PD-1 antibody.

Anti-PD-1 antibodies can include, without limitation, tislelizumab, pembrolizumab, and nivolumab. Tislelizumab is disclosed in U.S. Pat. No. 8,735,553. Pembrolizumab (formerly MK-3475), as disclosed by Merck in U.S. Pat. Nos. 8,354,509 and 8,900,587, is a humanized IgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NISCLC) and is under clinical investigation for the treatment of head and neck squamous cell carcinoma (UNSCC), and refractory Hodgkin's lymphoma (cHL). Nivolumab (as disclosed by Bristol-Meyers Squibb) is a fully human 1gG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma. In some embodiments, the anti-PD-1 antibody is tislelizumab.

### 7.5. Pharmaceutical Compositions and Formulations

Also provided are compositions, including pharmaceutical compositions, comprising an antibody drug conjugate described herein. These compositions can further comprise suitable carriers, such as pharmaceutically acceptable excipients including buffers, which are well known in the art.

Pharmaceutical compositions of an antibody drug conjugate as described herein may be prepared by mixing an antibody drug conjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyl dimethyl benzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYTLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycans such as chondroitinases.

Formulations to be used for in vivo administration are generally sterile. Sterility can be readily accomplished, e.g., by filtration through sterile filtration membranes.

### 7.6. Linker Payloads

Provided herein is a compound of Formula (IIa):

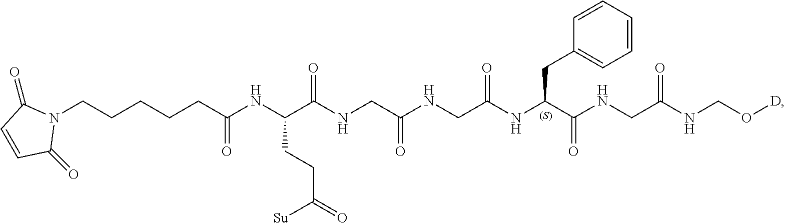

pharmaceutically acceptable salt thereof, wherein Su is a hydrophilic residue and D is a residue of a cytotoxic agent. In some embodiments, Su is HG-N(H)—, wherein HG is as described herein. In some embodiments, Su is

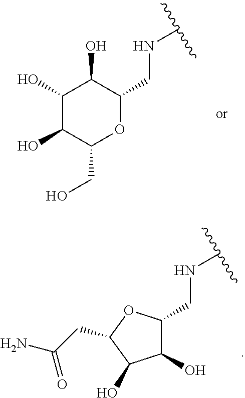 [SMILES: *NC[C@@H]1O[C@H](CO)[C@@H](O)[C@H](O)[C@H]1O ; *NC[C@H]1O[C@@H](CC(N)=O)[C@H](O)[C@@H]1O]

Also provided herein is a compound of Formula (Illa):

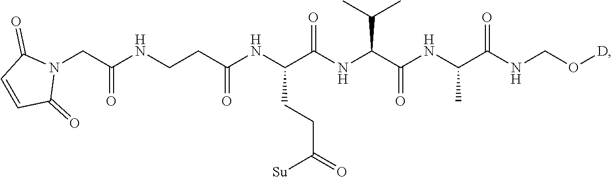

or a pharmaceutically acceptable salt thereof, wherein Su is a hydrophilic residue and D is a residue of a cytotoxic agent. In some embodiments, Su is HG-N(H)—, wherein HG is as described herein. In some embodiments, Su is

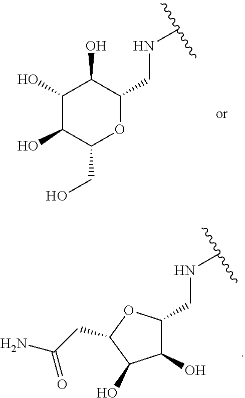 [SMILES: *NC[C@@H]1O[C@H](CO)[C@@H](O)[C@H](O)[C@H]1O ; *NC[C@H]1O[C@@H](CC(N)=O)[C@H](O)[C@@H]1O]

Any value for D described herein is suitable for use in connection with Formulas (IIa) and (IIIa). In some embodiments, D is

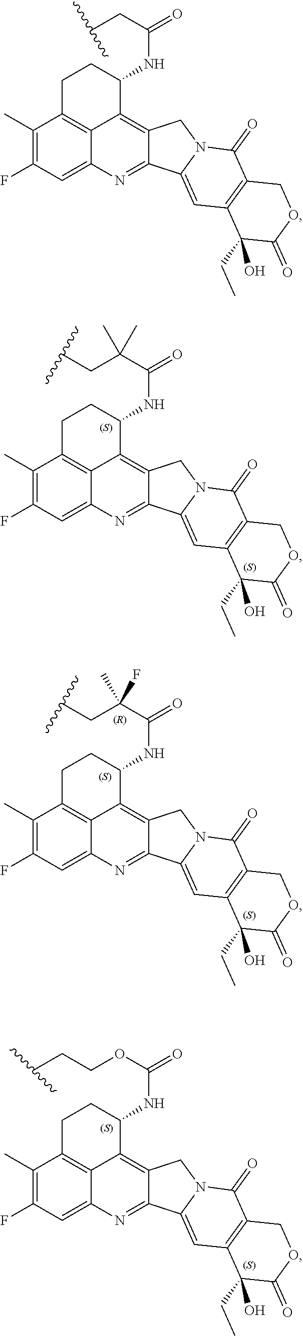

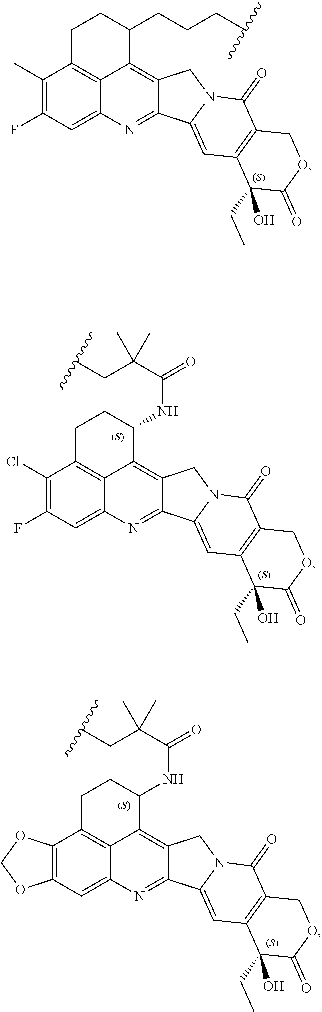

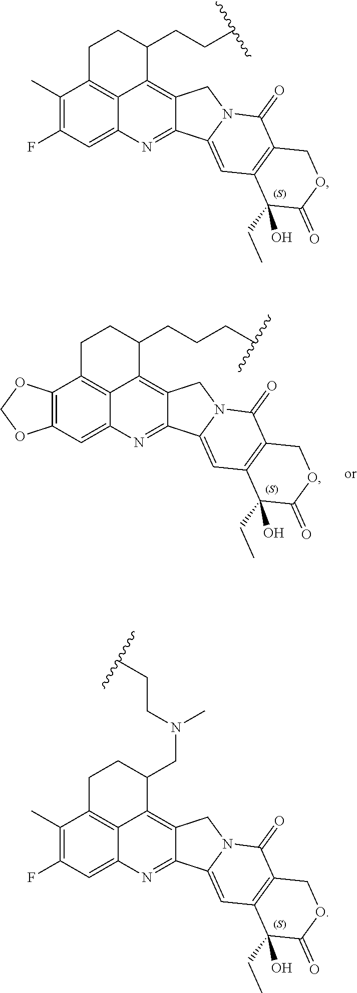

Some embodiments provide a linker payload of one of the following structures:

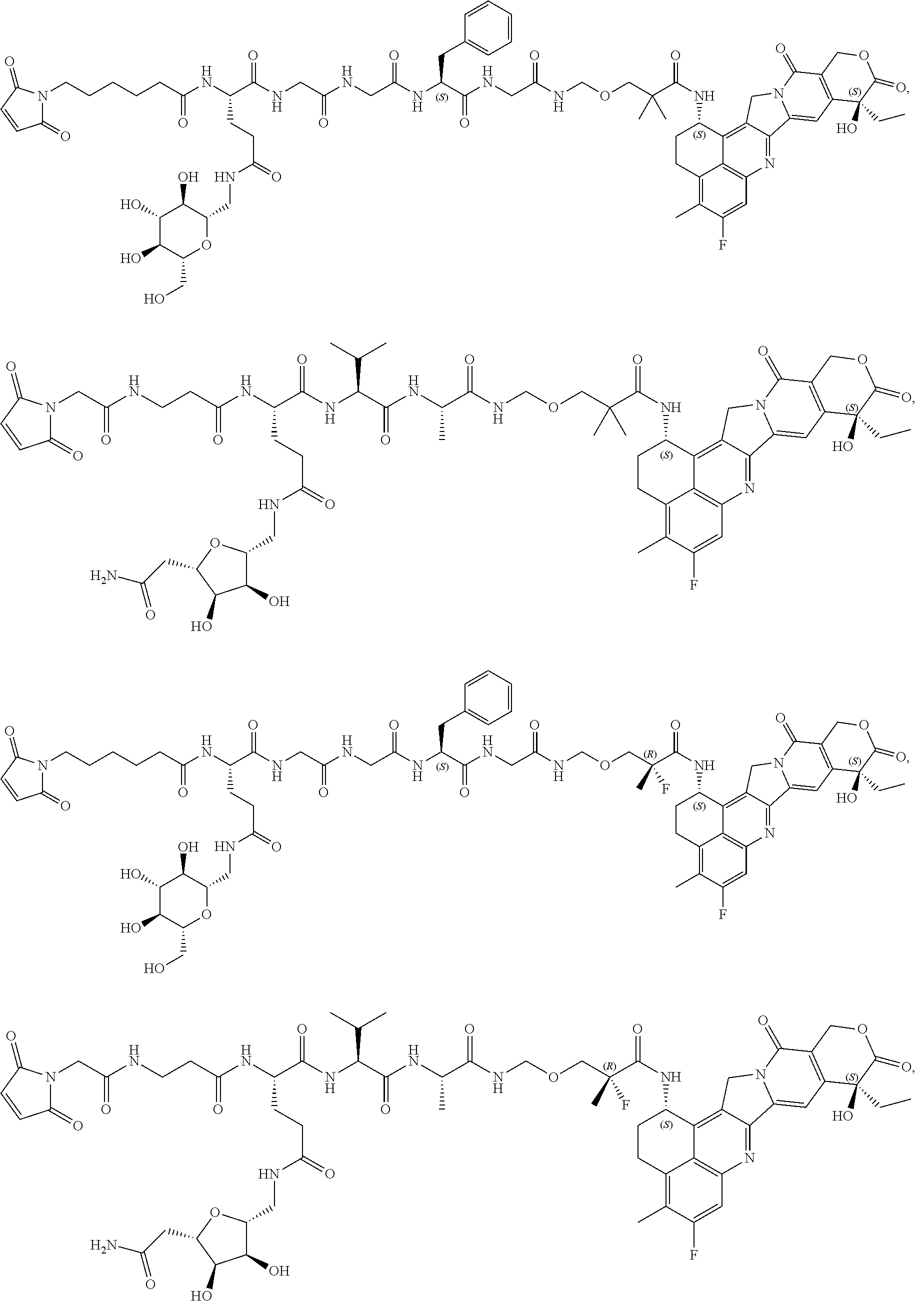

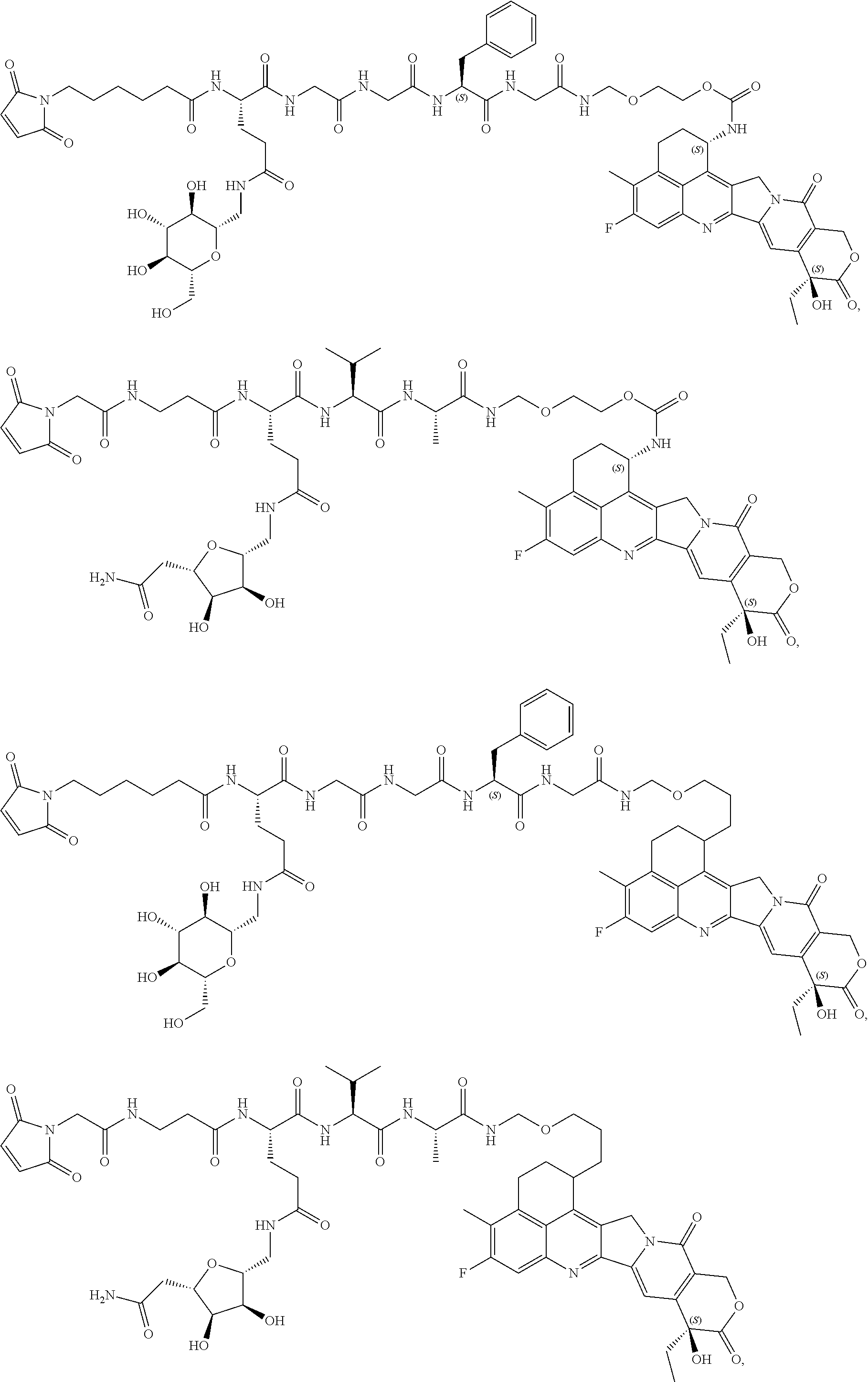

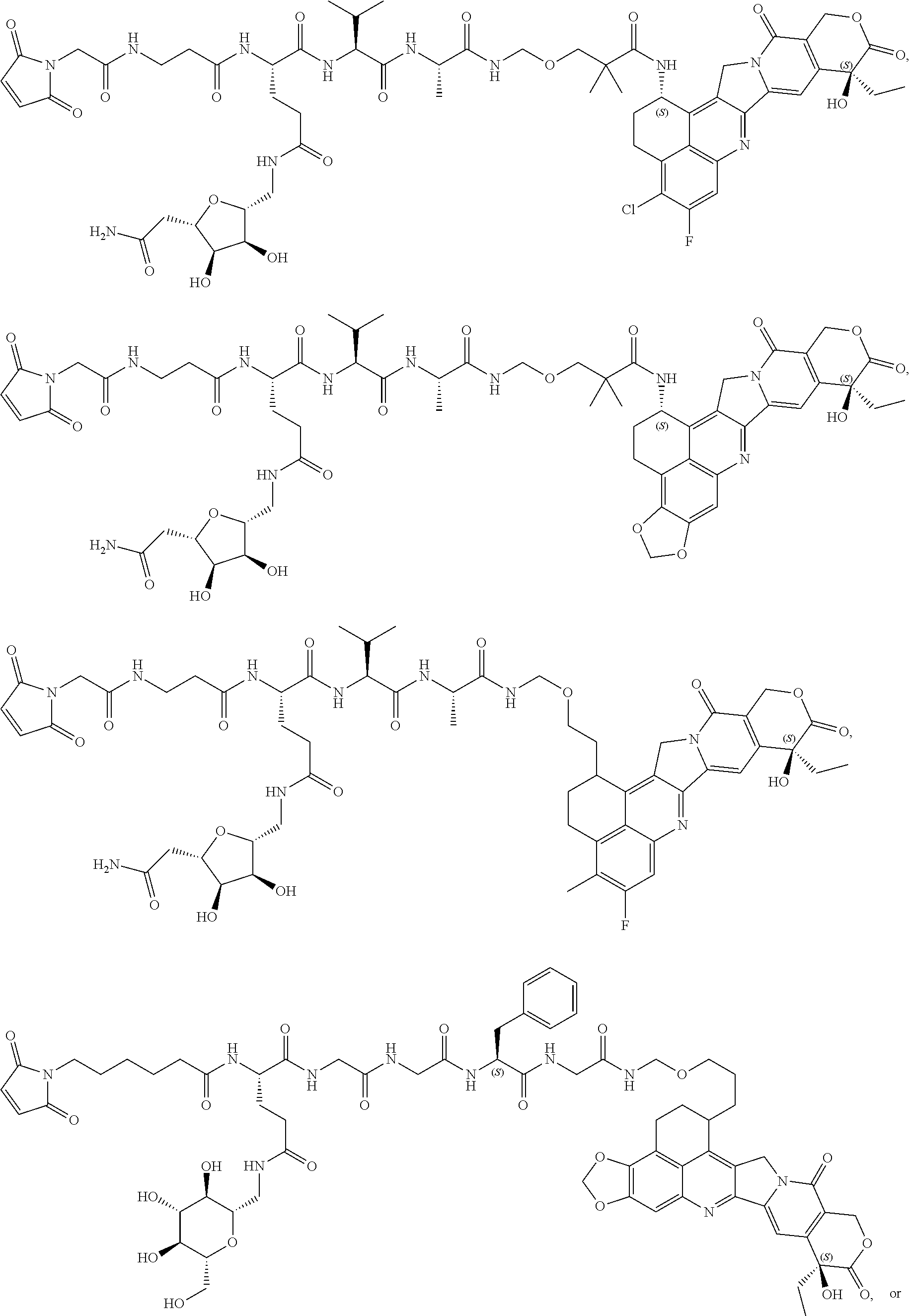

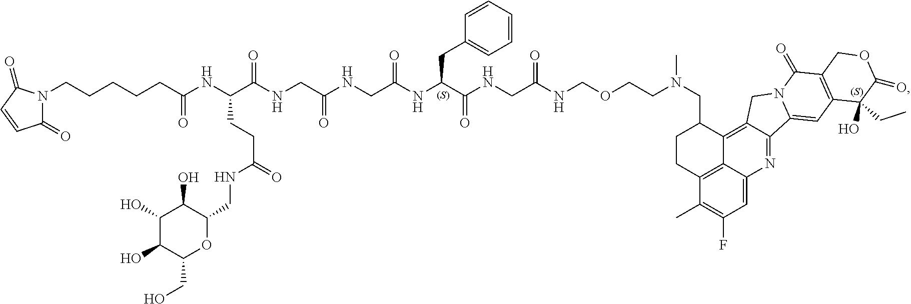

or a pharmaceutically acceptable salt thereof.

### 8. EXAMPLES

The examples below are intended to be purely exemplary and should not be considered limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v), and visualized by iodine or the solution of molybdophosphoric acid in ethanol unless otherwise specified. All extraction solvents, unless otherwise specified, were dried over anhydrous Na₂SO₄. ¹H NMR spectra were recorded on Bruck-400, Varian 400MR nuclear magnetic resonance spectrometer with TMS (tetramethylsilane) as the internal standard. Coupling constants were given in hertz. Peaks were reported as singlet (s), doublet (d), triplet (t), quartet (q), quintet (p), sextet (h), septet (hept), multiplet (m), or a combination thereof; br stands for broad. LC/MS data was recorded by using Agilenti 100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 18.0.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

In the following examples, the following abbreviations are used:

| | |
|---|---|
| THF | Tetrahydrofuran |
| MeOH | Methanol |
| HOBt | 1-Hydroxybenzotriazole |
| DIPEA | N,N-Diisopropylethylamine |
| DMF | N,N-Dimethylformamide |
| NH₄Cl | Ammonium chloride |
| HCl | Hydrochloric acid |
| TFA | Trifluoroacetic acid |
| Na₂SO₄ | Sodium sulfate |
| EtOAc | Ethyl acetate |
| prep-HPLC | Preparative high performance liquid chromatography |
| TBSCl | t-Butyldimethylchlorosilane |
| Et₂N | Diethylamine |
| r.t. | room temperature |
| MS | Mass spectrometry |
| ESI | Electron spray ionization |
| FA | Formic acid |
| NHS | N-hydroxysuccinimide |
| MTBE | Methyl tert-butyl ether |
| HPLC | High Performance Liquid Chromatography |
| DMSO | Dimethylsulfoxide |
| HATU | Hexafluorophosphate Azabenzotriazole Tetramethyl |
| | Uronium |
| TBAF | Tetrabutylammonium fluoride |
| PPTS | Pyridinium p-toluenesulfonate |

UPLC; analysis method:

Method A: Mobile phase A: 0.1% FA in water, B: MeCN; Gradient: 10% B maintain 0.2 min, 10%-95% B, 5.8 min, 95% B maintain 0.5 min; Flow rate: 0.6 mL/min; Column: ACQUITY UPLC® BEH C18 1.7 μm

Method B: Mobile phase A: 0.1% FA in water, B: MeCN; Gradient: 10% B maintain 0.5 min, 10%-90% B, 2.5 min, 90% B maintain 0.2 min; Flow rate: 0.6 mL/min; Column: ACQUITY UPLC® BEH C18 1.7 μm

Method C: Mobile phase A: 0.1% FA in water, B: MeCN; Gradient: 10% B maintain 0.2 min, 10%-90% B, 1.3 min, 90% B maintain 0.3 min; Flow rate: 0.6 mL/min; Column: ACQUITY UPLC® BEH C18 1.7 μm

### Example 1

### Example 1-1

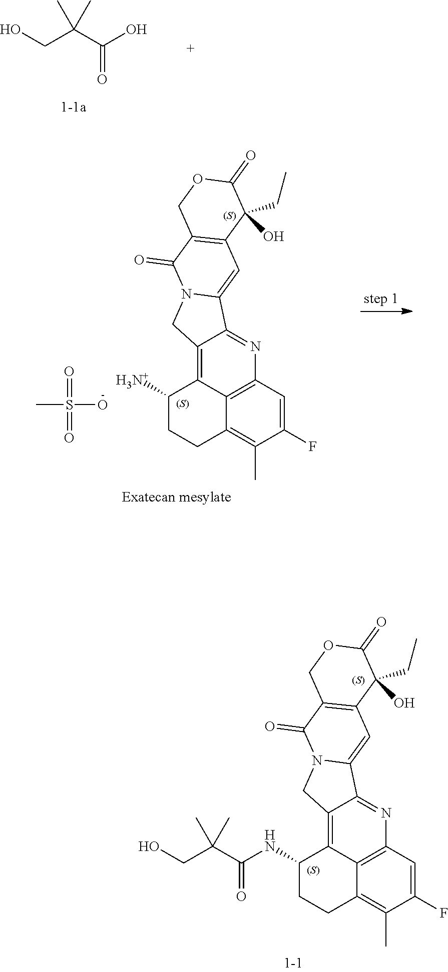

### Step 1

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxy-2,2-dimethylpropanamide (1-1)

To a mixture of 1-1a (5 mg, 0.042 mmol) and H ATU (16 mg, 0.042 mmol) in DMF (1 mL) were added DIPEA (21 μL, 16 mg, 0.13 mmol) and exatecan mesylate (purchased from ShangHai HaoYuan MedChemExpress CO. LTD, 23 mg, 0.043 mmol). The resulting brown mixture was stirred at r.t. for 2 h. On completion of the reaction, the mixture was purified by prep-HPLC (TFA) (Method: column:XBridge Prep C18 OBD 5 μm 19*150 mm; Mobile phase: A-water (0.1% TFA): B-acetonitrile; Flow rate: 20 mL/min). The fraction was lyophilized to give 1-1 (15 mg, 65.5% yield) as a white powder.

¹H NMR (400 MHz, DMSO-d₆): δ 8.00 (d, J=8.4 Hz, 1H), 7.79 (d, J=11.2 Hz, 1H), 7.31 (s, 1H), 6.52 (s, 1H), 5.59-5.54 (m, 1H), 5.42 (s, 2H), 5.18 (q, J=19.2 Hz, 2H), 4.87 (t, J=5.2 Hz, 1H), 3.45 (dd, J=10.2, 4.8 Hz, 1H), 3.41-3.28 (m, 1H), 3.15 (t, J=5.6 Hz, 2H), 2.40 (s, 3H), 2.24-2.07 (m, 2H), 1.92-1.80 (m, 2H), 1.11 (d, J=7.6 Hz, 6H), 0.87 (t, J:=7.2 Hz, 31H). MS (ESI) m/z: 536.4 [M+H]⁺.

### Example 1-2

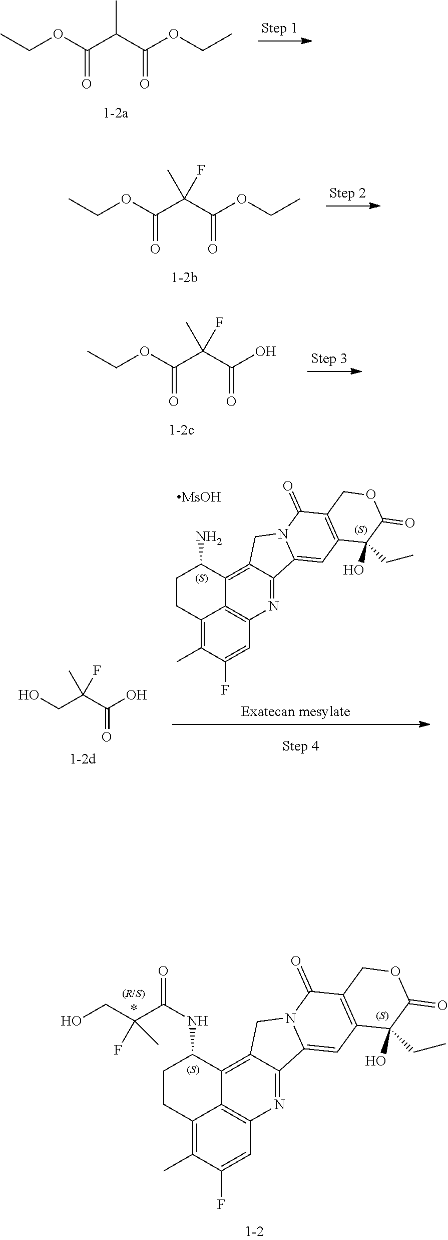

### Step 1

### Diethyl 2-fluoro-2-methylmalonate (1-2b)

A solution of compound 1-2a (10.0 g, 57.4 mmol) in THF (200 mL) was cooled to 0° C. 60% of NaH in oil (3.21 g, 80.37 mmol) was added into the mixture portion-wise, stirred at 0° C. for 30 mi. Then N-fluoro-N-(phenylsulfonyl)benzenesulfonamide (NSFI, 19.91 g, 63.2 mmol) was added into the mixture portion-wise at 0° C. Then warmed up to r.t. and stirred for 16 h. After reaction completed, the suspension was filtered and the filtrated was concentrated. PE (100 mL) was added into the residue, the precipitate was filtered and the filtrate was concentrated to give compound 1-2b (12.5 g, crude) as a light-yellow oil.

¹H NMR (400 MHz, CDCl₃) δ 4.30 (q, J=7.2 Hz, 4H), 1.79 (d, J=22.0 Hz, 3H), 1.31 (t, J=7.2 Hz, 6H); ¹⁹F NMR (376 MHz, CDCl₃) δ −157.50.

### Step 2

### 3-ethoxy-2-fluoro-2-methyl-3-oxopropanoic acid (1-2c)

To a solution of compound 1-2b (1.0 g, 5.2 mmol) in EtOH (5 mL) was added KOH solution (321 mg) in H₂O (50 μL) and EtOH (2 mL) dropwise at 0° C. The mixture was stirred at r.t. for 2 h. The mixture was diluted with 20 mL, washed with DCM (20 mL*3). The aqueous solution was adjusted to pH=3 by 1 N HCl, then extracted by EtOAc (50 mL*3). The organic layer was combined and dried over anhydrous Na₂SO₄, filtered, and concentrated to give compound 1-2c (470 mg, 550% yield) as a colorless oil.

¹H NMR (400 MHz, CDCl₃) δ 8.31 (br s, 1H), 4.32 (q, J=7.2 Hz, 2H) 1.83 (d, J=22.0 Hz, 3H), 1.33 (t, J=7.2 Hz, 31); ¹⁹F NMR (376 MHz, CDCl₃) -157.59.

### Step 3

### 2-fluoro-3-hydroxy-2-methylpropanoic acid (1-2d)

To a solution of compound 1-2c (200 mg, 1.22 mmol) in isopropanol (4 mL) was added 2 M LiBH4 (1.22 mL, 2.44 mmol) at 0° C. The mixture was stirred at r.t. for 2 h. The mixture was quenched by 2 N HCl (1.22 mL) dropwise at 0° C. The diluted with H₂O (10 mL), and extracted with EtOAc (50 mL*3). The organic layer was combined and dried over anhydrous Na₂SO₄, filtered and concentrated to give compound 1-2d (92 mg, 61.7% yield) as a colorless oil.

¹H NMR (400 MHz, CDCl₃) δ 4.01-3.81 (m, 2H), 1.58 (d, J=21.2 Hz, 3H); ¹⁹F NMR (376 MHz, CDCl₃) δ −163.98.

### Step 4

### N-((1S,9S)-9-ethyl-5-fluoro-9-hydroxy-4-methyl-10,13-dioxo-2,3,9,10,13,15-hexahydro-11-1H,12H-benzo[de]pyrano[3′,4′:6,7]indolizino[1,2-b]quinolin-1-yl)-2-fluoro-3-hydroxy-2-methylpropanamide (1-2)

To a solution of compound 1-2d (23 mg, 0.19 mmol) in DMF (2 mL) were added exatecan mesylate (50 mg, 0.094 mmol), HATU (54 mg, 141 mmol), and DIEA (36 mg, 0.28 mmol). The mixture was stirred at r.t. for 1 h. The mixture was purified by prep-HPLC (FA) (Method: column: XBridge Prep C18 OBD 5 μm 19*150 mm; Mobile phase: A-water (0.1% formic acid): B-acetonitrile; Flow rate: 20 mL/min) The fraction was lyophilized to give the following two isomers:

1-2-1: white solid, (11 mg, 21.9% yield). UPLC-MS, RT=3.52 min.

¹H NMR (400 MHz, DMSO-d₆) δ 9.06 (dd, J=9.0, 2.8 Hz, 1H1), 8.00 (d, J=10.9 Hz, 1H), 7.54 (s, 1H), 6.75 (s, 1H), 5.82 (d, J=8.0 Hz, 1H), 5.65 (s, 21), 5.43 (dt, J=77.8, 12.4 Hz, 3H), 4.17-3.91 (m, 11H), 3.83 (ddd, J=18.0, 12.4, 5.6 Hz, 1H), 3.40-3.27 (m, 1H), 2.62 (s, 3H), 2.50-2.34 (m, 2H), 2.22-1.98 (m, 2H), 1.81 (d, 0.1=21.4 Hz, 3H), 1.11 (t, J=7.2 Hz, 3H); MS (ESI) m/z: 540.3 [M+H]⁺.

Isomer 2: white solid, 1-2-2 (8.4 mg, 16.6% yield). UPLC-MS, RT=3.86 min.

¹H NMR (400 MHz, DMSO-d₆) δ 8.72 (dd, J=8.4, 2.4 Hz, 1H), 7.78 (d, J=11.2 Hz, 1H), 7.31 (s, 1H), 6.52 (s, 1H), 5.58 (d, J=8.0 Hz, 1H), 5.42 (s, 2H), 5.32-5.05 (m, 3H), 3.83 (dd, J=26.8, 12.0 Hz, 1H), 3.61 (dd, J=21.6, 12.0 Hz, 1H), 3.22-3.07 (m, 2H), 2.46-2.30 (m, 3H), 2.28-2.05 (m, 2H), 2.02-1.74 (n, 2H), 1.45 (d, J=21.4 Hz, 3H), 0.87 (t, J=7.2 Hz, 31); MS (ESI) m/z: 540.3 [M+H]⁺.

### Example 1-3

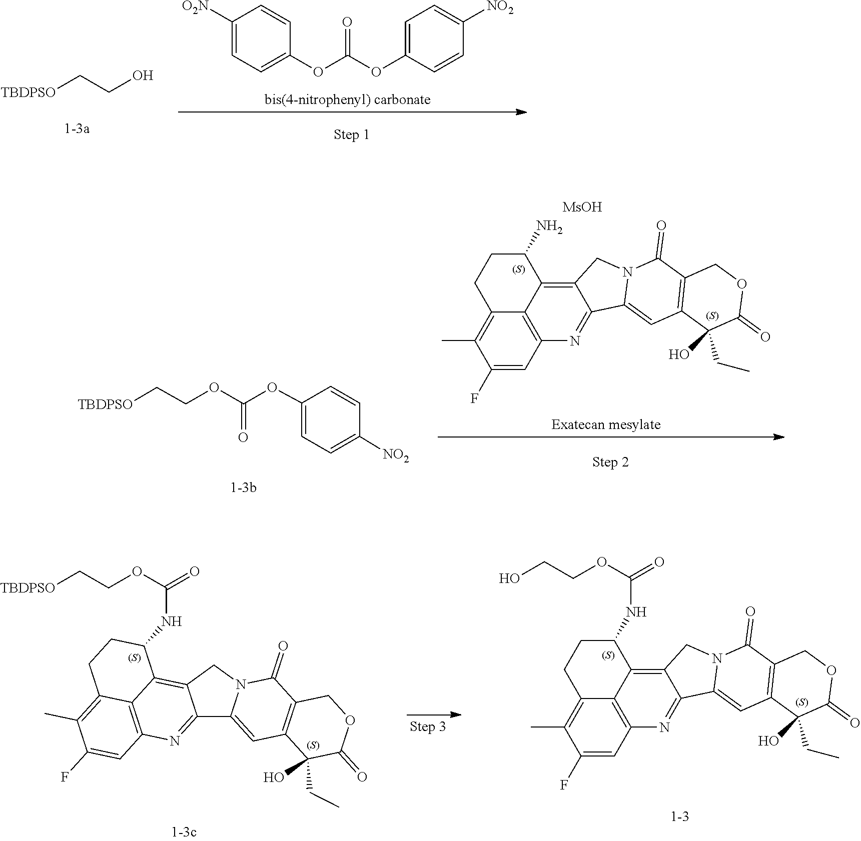

### Step 1

### 2-((tert-butyldiphenylsilyl)oxy)ethyl (4-nitrophenyl) carbonate (1-3b)

To the solution of 1-3a (100 mg, 0.33 mmol) and bis(4-nitrophenyl) carbonate (123 mg, 0.40 mmol) in dry CH₂Cl₂ (2 mL) was added DIEA (176 μL, 1.0 mmol) and DM AP (4.1 mg, 0.033 mmol), stirred at r.t. overnight. The solution was poured into IN HCl (2 mL), and extracted with CH₂Cl₂ (2 mL*3). The organic phase was concentrated and purified by flash column chromatography (silica gel, petroleum ether/ethyl acetate=5:1) to give compound 1-3b (148 mg, 95.5% yield) as a colorless oil.

¹H NMR (400 MHz, CDCl₃) δ 8.27 (d, J=9.1 Hz, 2H), 7.69 (d, J=6.5 Hz, 4H), 7.50-7.31 (m, 8H), 4.47-4.36 (m, 2H), 3.99-3.89 (m, 2H), 1.07 (s, 9H).

### Step 2

### 2-((tert-butyldiphenylsilyl)oxy)ethyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (1-3c)

To a solution of exatecan mesylate (50 mg, 0.094 mmol), 1-3b (53 mg, 0.11 mmol), and HOBt (1.3 mg, 0.009 mmol) in dry DMF (1 mL) was added DIEA (50 μL, 0.28 mmol), stirred at r.t. overnight. The solution was poured into sat. NH₄Cl (5 mL), extracted with EtOAc (5 mL*3). The organic phase was concentrated and purified by flash column chromatography (silica gel, petroleum ether/ethyl acetate=1:3) to give compound 1-3c (68 mg, 94.9% yield) as a yellow solid.

MS (ESI) m/z: 762.4 [M+H]⁺.

¹H NMR (400 MHz, CDCl₃) δ 7.70-7.60 (m, 5H), 7.59-7.52 (m, 1H), 7.36-7.27 (m, 7H), 5.68 (d, J=16.2 Hz, 1H), 5.33 (d, J=16.3 Hz, 1H), 5.25-5.15 (m, 1H), 5.09-4.96 (m, 2H), 4.56-4.44 (m, 1H), 4.33-4.21 (m, 1H), 4.04-3.89 (m, 2H), 3.70 (s, 1H), 3.17-2.99 (m, 2H), 2.47-2.34 (m, 4H), 2.19-2.07 (m, 1H), 2.05-1.88 (m, 2H), 1.08 (t, J=7.3 Hz, 31H), 1.04 (s, 9H).

### Step 3

### 2-hydroxyethyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (1-3)

To a solution of 1-3e (65 mg, 0.085 mmol) in dry THE (2 mL) was added IM TBAF in THF (102 μL, 0.102 mmol) at 0° C., stirred at r.t. for 40 min. The solution was poured into sat. NH₄Cl (5 mL), extracted with CH₂Cl₂/MeOH (5/1, 6 mL*3). The organic phase was concentrated and purified by flash column chromatography (silica gel, CH₂Cl₂/MeOH=10:1) to give compound 1-3 (39 mg, 87.3% yield) as a pale yellow solid.

MS (ESI) m/z: 524.4 [M+H]⁺.

¹H NMR (400 MHz, DMSO-d₆) δ 7.98 (d, J:=8.8 Hz, 1H), 7.77 (d, J=10.9 Hz, 1H), 7.31 (s, 1H), 6.52 (s, 1H), 5.43 (s, 2H), 5.33-5.17 (m, 3H), 4.78 (t, J=5.4 Hz, 1H), 4.19-4.01 (m, 2H), 3.68-3.57 (m, 2H), 3.30-320 (m, 1H), 3.17-3.06 (m, 1H), 2.38 (s, 3H), 2.26-2.07 (m, 2H), 1.95-1.78 (m, 2H), 0.87 (t, J=7.4 Hz, 3H).

### Example 1-4

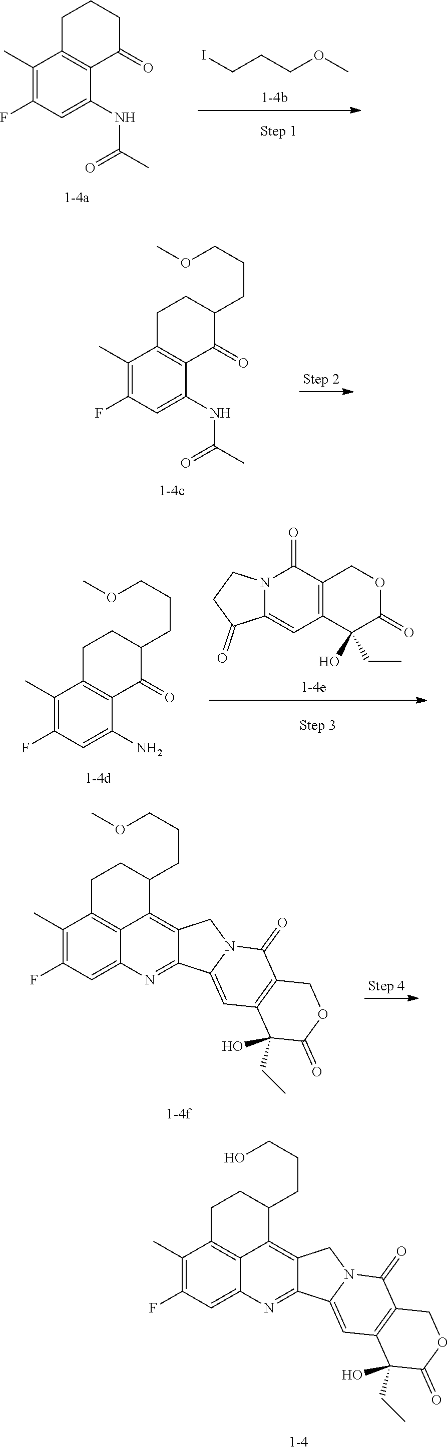

### Step 1

### N-(3-fluoro-7-(3-methoxypropyl)-4-methyl-8-oxo-5,6,7,8-tetrahydronaphthalen-1-yl)acetamide (1-4c)

To a solution of LDA (1.40 mL, 2.81 mmol, 2M in THF) in THE (19 mL) was added 1-4a (300.00 mg, 1.28 mmol) at −78° C., and the mixture was stirred at the same temperature for 2 h before a solution of 1-4b (0.38 g, 1.91 mmol) in THE (1 mL) was added dropwise. The reaction mixture was then slowly warmed up to 0° C. and stirred continuously for 4 h. The reaction was subsequently quenched with saturated aqueous NH₄Cl solution (50 mL), and the organic materials were extracted with EtOAc (30 mL*3) The combined organic layers were washed with brine (50 mL) and dried over MgSO₄ before the combined extracts were concentrated in vacuo. The resulting crude residue was purified by flash column chromatography (silica gel, Hex:EtOAc=97:3) to give 1-4c (80.00 mg, 20.41% yield) as a colorless oil.

MS (ESI) m/z: 308.2 [M+H]f.

### Step 2

### 8-amino-6-fluoro-2-(3-methoxypropyl)-5-methyl-3,4-dihydronaphthalen-1(2H)-one (1-4d)

2N HCl (20 mL) was added to a solution of 1-14f (836 mg, 2.72 mmol) in MeOH (20 mL). The reaction mixture was purged with N₂ for three times and reacted at 60° C. under N₂ for 5 h. The mixture was concentrated and purified by silica gel column chromatography (CH₂Cl₂: MeOH) to provide 1-4d (652 mg, 90.3% yield) as a gray solid.

MS (ESI) m/z: 266.2[M+H]⁺.

### Step 3

### (9S)-9-ethyl-5-fluoro-9-hydroxy-1-(3-methoxypropyl)-4-methyl-1,2,3,9,12,15-hexahydro-10H,13H-benzo[de]pyrano[3′,4′:6,7]indolizino[1,2-b]quinoline-10,13-dione (1-4f)

1-4d (650 mg, 2.45 mmol), 1-4e (787 mg, 2.99 mmol) and a catalytic amount of PPTS (200 mg, 0.80 mmol) in toluene (40 mL) was heated to reflux (135˜140° C.) using a Dean-Stark trap for 48 h. The reaction was concentrated under vacuum and purified by silica gel column chromatography (CH₂Cl₂: MeOH) to provide 1-4f (857 mg, 71.0% yield) as a gray solid. UPLC analysis: 1-4f, peak 1, retention time=2.66 min; peak 2, retention time=2.77 min (Mobile phase A: 0.1% FA in water, B: MeCN; Gradient: 10% B maintain 1 min, 10%-95% B, 5 min, 95% B maintain 1 min; Flow rate: 0.6 mL/min; Column: ACQUITY UPLC® BEH C18 1.7 μm).

MS (ESI) m/z: 493.1 [M+H]⁺.

### Step 4

### (9S)-9-ethyl-5-fluoro-9-hydroxy-1-(3-hydroxypropyl)-4-methyl-1,2,3,9,12,15-hexahydro-10H,13H-benzo[de]pyrano[3′,4′:6,7]indolizino[1,2-b]quinoline-10,13-dione (1-4)

To a solution of 1-4f (70.00 mg, 0.14 mmol) in CH₂Cl₂ (20 mL) was added BBr₃ (71.26 mg, 0.28 mmol) at 0° C., and the mixture was stirred at the same temperature for 1 h. The reaction mixture was then slowly warmed up to 25° C. and stirred continuously for 3 h. The reaction was subsequently quenched with saturated aqueous NaHCO₃ solution and the organic materials were extracted thrice with EtOAc (30 mL*3). The combined organic layers were washed with brine (50 mL) and dried over MgSO₄ before the combined extracts were concentrated in vacuo. The resulting crude residue was purified by flash column chromatography (silica gel, CH₂Cl₂: MeOH=90:10) to give 1-4 (4.20 mg, 6.18% yield) as a gray solid.

MS (ESI) m/z: 479.4 [M+H]⁺.

UPLC analysis: 1-4-1, peak 1, retention time=4.49 min; 1-4-2, peak 2, retention time=4.65 min (Mobile phase A: 0.1% FA in water, B: MeCN; Gradient: 15% B maintain 1 min, 15%-95% B, 9 min, 95% B maintain 2 min; Flow rate: 0.6 mL/min; Column: ACQUITY UPLC® BEH C18 1.7 μm).

1-4-1: ¹H NMR (400 MHz, DMSO-d6) δ 7.74 (d, J=11.1 Hz, 1H), 7.30 (s, 1H), 6.51 (s, 1H), 5.43 (s, 2H), 5.36 (d, J=18.7 Hz, 11H), 5.22 (d, J=18.7 Hz, 11H), 352-3.39 (m, 2H), 3.18-2.97 (m, 2H), 2.38 (s, 31H), 2.34-2.25 (m, 2H), 2.01-1.78 (m, 3H), 1.78-1.51 (m, 4H), 0.87 (t, J=7.3 Hz, 3H).

1-4-2: ¹H NMR (400 MHz, DMSO-d6) δ 7.74 (d, J=11.1 Hz, 1H), 7.30 (s, 1H), 6.52 (s, 1H), 5.43 (s, 2H), 5.37 (d, J=18.8 Hz, 1H), 5.23 (d, J=18.7 Hz, 1H), 3.51-3.40 (m, 2H), 3.16-2.98 (m, 2H), 2.38 (s, 3H), 2.35-2.24 (n, 2H), 1.99-1.81 (m, 3H), 1.78-1.53 (m, 4H), 0.87 (t, J=7.3 Hz, 3H).

### Example 1-5

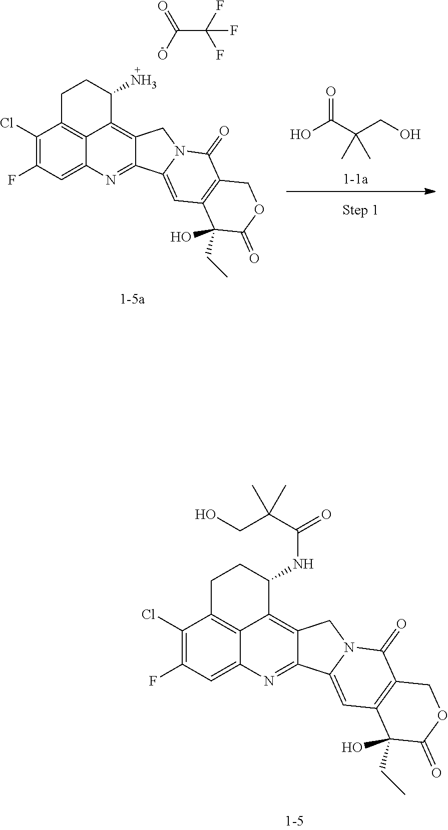

### (1S,9S)-4-chloro-9-ethyl-5-fluoro-9-hydroxy-10,13-dioxo-2,3,9,10,13,15-hexahydro-11,12H-benzo[de]pyrano[3′,4′:6,7]indolizino[1,2-b]quinolin-1-aminium 2,2,2-trifluoroacetate (1-5a)

1-5a was synthesized according to the reported procedure (U.S. Pat. No. 11,318,212 B2).

N-((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)-3-hydroxy-2,2-dimethylpropanamide (1-5)

1-5 (5.8 mg, 99.82% purity, 67.9% yield) was synthesized according to a similar procedure as that of step 1 of Example 1-1.

MS (ESI) m/z: 556.3 [M+H]⁺.

### Example 1-6

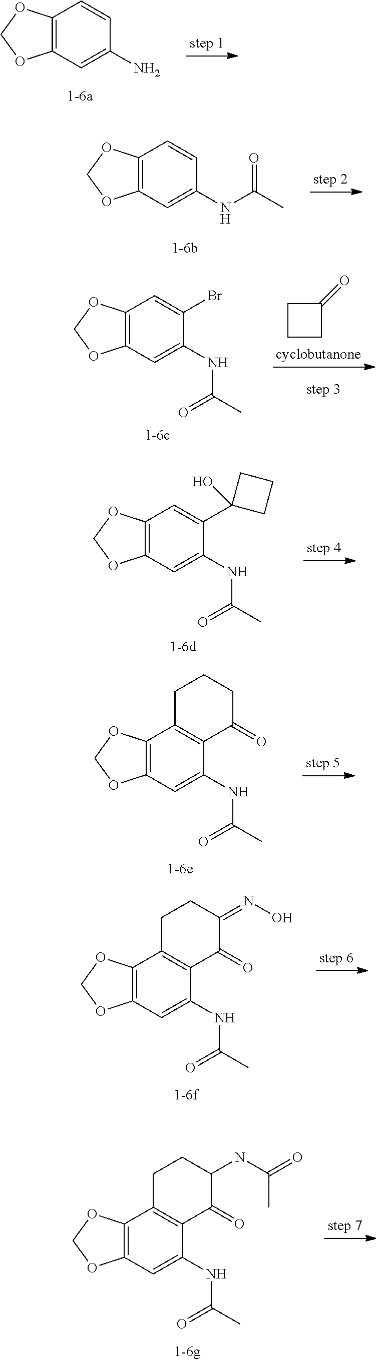

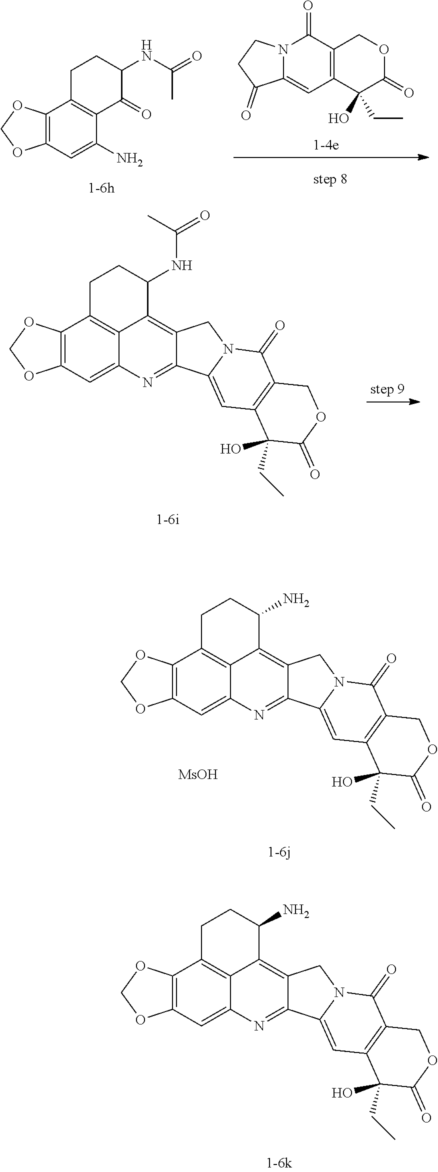

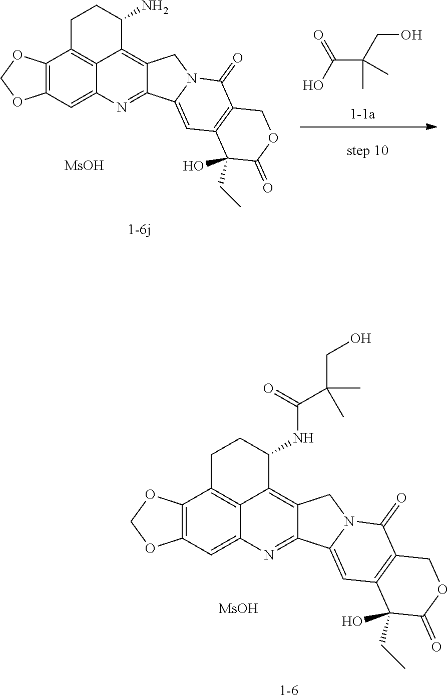

### Step 1

### N-(benzo[d][1,3]dioxol-5-yl)acetamide (1-6b)

To a solution of 1-6a (25.00 g, 182 mmol) in CH₂Cl₂ (200 mL) was added Ac₂O (27.85 g. 273 mmol) and Et₃N (36.76 g, 364 mmol) at 0° C. The reaction mixture was reacted at 20° C. for 3 h. LCMS showed the reaction was completed. The reaction mixture was concentrated to 50 mL. To the residual was added EtOAc (200 mL) and washed with saturated NaHCO₃ (200 mL*3). The organic layer was dried over Na₂SO₄ and filtered. The filtrate was concentrated under vacuum and the residue was used in the next step without further workup and purification (28.25 g, crude).

MS (ESI) m/z: 180.1 [M+H]⁺.

### Step 2

### N-(6-bromobenzo[d][1,3]dioxol-5-yl)acetamide (1-6c)

A solution of 1-6b (28.25 g, crude) and sodium acetate (15.4 g, 188.3 mmol) in acetic acid (100 mL) was heated to 60° C., then a mixture of bromine (30.1 g, 188.3 mmol) and acetic acid (60 mL) were added to the reaction solution dropwise. The reaction temperature was raised to 80° C. and stirred at 80° C. for 2 h. LCMS showed the reaction was completed, the reaction solution was poured into ice water, and a yellow solid was generated. The solid was filtered and washed with water three times to obtain a crude product which was recrystallized with EtOH to afford 1-6c (17.1 g, 42% yield).

MS (ESI) m/z: 258.1/260.1 [M+H]⁺.

1H NMR (400 MHz, DMSO) δ 9.36 (s, 1H), 7.22 (s, 1H), 7.08 (d, J=3.7 Hz, 1H1), 6.07 (s, 2H), 2.02 (s, 3H).

### Step 3

### N-(6-(1-hydroxycyclobutyl)benzo[d][1,3]dioxol-5-yl)acetamide (1-6d)

A solution of compound 1-6c (8.5 g, 33.01 mmol) in THF (200 mL) was cooled to −90° C., n-BuLi (15.84 mL, 39.60 mmol) was added into the mixture over 2 h under N₂ protection. The internal temperature was kept under −85° C. The mixture was stirred at −85° C. for 20 min. A solution of cyclobutanone (2.7 g, 39.60 mmol) in THF (50 mL) was added into the mixture dropwise. The mixture was stirred at −85° C. for 30 min and warmed to room temperature for 1 h. The mixture was quenched by sat. NH₄Cl solution (200 mL), extracted with EtOAc (150 mL*3). The combined organic layer was dried over Na₂SO₄. After filtration and evaporation, the residue was washed with MTBE (5 mL*3) and recrystallized with EtOH to afford 1-6d (3.1 g, 37.5% yield).

MS (ESI) m/z: 250.1 [M+H]⁺.

### Step 4

### N-(6-oxo-6,7,8,9-tetrahydronaphtho[1,2-d][1,3]dioxol-5-yl)acetamide (1-6e)

To a solution of compound 1-6d (3.1 g, 12.44 mmol) in CH₂Cl₂ (20 mL) and H₂O (20 mL) were added AgNO₃ (12.4 ml, 6.22 mmol), K₂S₂O₈ (8.4 g, 31.1 mmol). The mixture was stirred at 20° C. for 16 h. The mixture was filtered through celite and the filtration residue was washed with CH₂Cl₂:MeOH=1:1 (50 mL*3). The filtrate was poured into water and extracted with CH₂Cl₂ (300 mL*3). The combined organic layer was dried over Na₂SO₄. After filtration and evaporation, the residue was purified by silica column gel chromatography (eluent: petroleum ether/CH₂Cl₂=100/0 to 0/100) to afford 1-6e (1.9 g, 61.8% yield) as a light-yellow solid.

MS (ESI) m/z: 248.1 [M+H]⁺.

### Step 5

### (Z)—N-(7-(hydroxyimino)-6-oxo-6,7,8,9-tetrahydronaphtho[1,2-d][1,3]dioxol-5-yl)acetamide (1-6f)

To a solution of compound 1-6e (1.9 g, 7.69 mmol) in THF (20 mL) and t-BuOH (5 ml) was added t-BuOK (1 M in THF, 9.28 mL, 9.28 mmol) dropwise at 0° C., The atmosphere was purged with N₂ and the mixture was cooled to 0° C. using an ice water bath. After 5 mins, to the stirred mixture was added isopentyl nitrite (1.09 g, 9.28 mmol). LCMS showed the reaction was completed and the reaction mixture was allowed to warm to r.t. 1 N HCl was added to adjusted pH to 1. The aqueous layer was extracted with CH₂Cl₂: THF (2:1, v/v, 50 mL*3). The combined organic layer was washed with brine (100 mL) and dried over Na₂SO₄. After filtration and evaporation, the residue was triturated with MTBE (20 mL*2) to afford 1-6f (1.5 g, 70.6% yield).

MS (ESI) m/z: 277.1 [M+H]⁺.

### Step 6

### N,N′-(6-oxo-6,7,8,9-tetrahydronaphtho[1,2-d][1,3]dioxole-5,7-diyl)diacetamide (1-6g)

To a solution of compound 1-6f (1.5 g, 5.43 mmol) in Ac₂O (5 mL) was added PtO₂ (150 mg). The mixture was stirred at 20° C. under H₂ (15 psi) for 16 h. LCMS showed the reaction was completed. The mixture was filtered through a pad of celite. The filtrate was diluted with EtOAc (100 mL) and washed by sat. NaHCO₃ (100 mL*3). The organic layer was dried over Na₂SO₄. After filtration and evaporation, the residue 1-6g (1.3 g, crude) was used in the next step without further workup and purification.

MS (ESI) m/z: 305.2 [M+H]⁺.

### Step 7

### N-(5-amino-6-oxo-6,7,8,9-tetrahydronaphtho[1,2-d][1,3]dioxol-7-yl)acetamide (1-6h)

To a solution of compound 1-6g (1.3 g, crude) in MeOH (10 mL) was added HCl (2 N, 10 mL). The mixture was stirred at 60° C. for 3 h. The mixture was cooled to r.t. and adjusted pH to 8 using sat. NaHCO₃. The mixture was extracted with EtOAc (50 mL*3). The organic layer was dried and concentrated. The residue was purified by a silica gel column chromatography (eluent: CH₂Cl₂/MeOH=100/0 to 90/10) to give compound 1-6h (670 mg, 59.8% yield) as an off-white solid.

MS (ESI) m/z: 263.1 [M+H]⁺.

### Step 8

### N-((10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)acetamide (1-6i)

To a solution of compound 1-6h (400 mg, 1.52 mmol) in toluene (40 mL) and o-cresol (0.5 mL) was added 1-4e (479 mg, 1.82 mmol) and PPTS (38 mg, 0.15 mmol). The mixture was refluxed at 140° C. for 24 h. LCMS showed the reaction was completed and black solid precipitated. After filtration, the filter cake was washed with acetone (10 mL*3) to give a dark brown solid 1-6i (580 mg, crude) which was used in the next step without further workup and purification.

MS (ESI) m/z: 490.3 [M+H1]⁺.

### Step 9

### (1S,10S)-1-amino-10-ethyl-10-hydroxy-1,2,3,10,13,16-hexahydro-11H,141-1-benzo[de][1,3]dioxolo[4,5-g]pyrano[3′,4′:6,7]indolizino[1,2-b]quinoline-11,14-dione (1-6j)

### (1R,10S)-1-amino-O-ethyl-10-hydroxy-1,2,3,10,13,16-hexahydro-11H,14H-benzo[de][1,3]dioxolo[4,5-g]pyrano[3′,4′:6,7]indolizino[1,2-b]quinoline-11,14-dione (1-6k)

A solution of compound 1-6i (580 mg, crude) in MsOH (10 mL) and H₂O (10 mL) was refluxed at 110° C. for 5 h. LCMS showed the reaction was completed. The mixture was filtered and purified by prep-HPLC (Method: column: XBridge Prep C18 OBD 5 μm 19*150 mm; Mobile phase: A-water (0.1% TFA): B-acetonitrile; Flow rate: 20 mL/min). The fraction was lyophilized to give the following two isomers:

Compound 1-6j (110 mg, 20.8% yield) was obtained as yellow solid.

MS (ESI) m/z: 448.2 [M+H]⁺. Retention time (0.82 min).

Compound 1-6k (120 mg, 22.7% yield) was obtained as yellow solid.

MS (ESI) m/z: 448.2 [M+H]⁺. Retention time (1.87 min).

N-((1S,10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)-3-hydroxy-2,2-dimethylpropanamide (1-6)

Compound 1-6 (1.8 mg, 17.9% yield) was synthesized according to the synthetic procedure of step 1 of example 1-1.

MS (ESI) m/z: 548.4 [M+H]⁺.

### Example 1-7

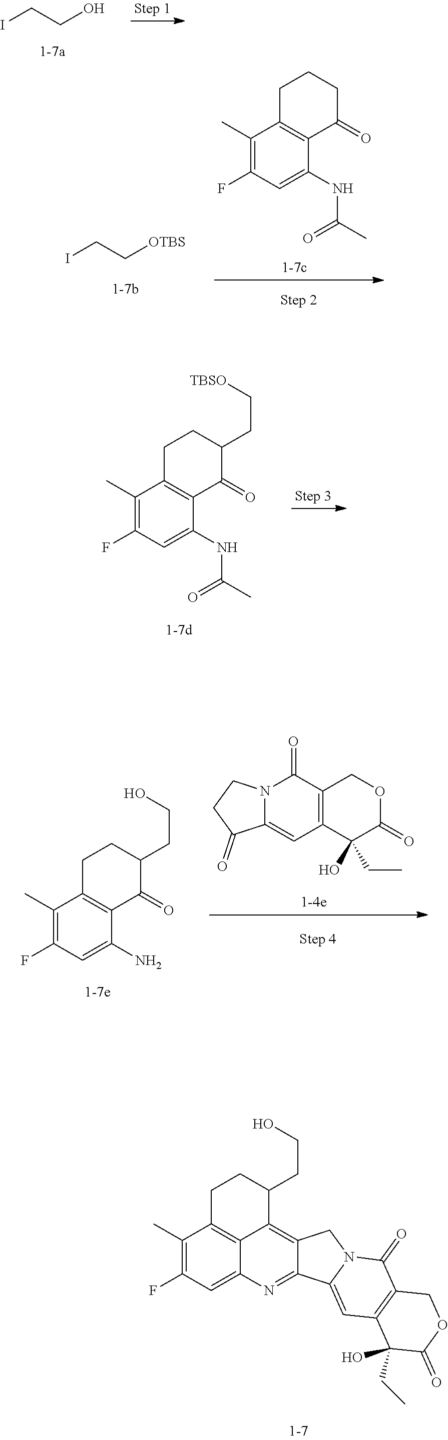

### Step 1

### tert-butyl(2-iodoethoxy)dimethylsilane (1-7b)

A solution of compound 1-7a (3000 mg, 17.44 mmol) in CH₂Cl₂ (40 mL) was cooled to 0° C., then imidazole (1779 mg, 26.16 mmol) and TBSCl (3139 mg, 20.93 mmol) was added portion-wise at 0° C. The mixture was stirred at 20° C. for 16 h. TLC (SiO₂, petroleum ether) showed the reaction was completed. The reaction was poured into water (40 mL) and extracted with CH₂Cl₂ (50 mL*3). The combined organic layer was dried over Na₂SO₄ and concentrated to give the residue which was purified by silica column gel chromatography (eluent: petroleum ether=100) to afford 1-7b (3.5 g, 70.2% yield) as a colorless oil.

### Step 2

### N-(7-(2-((tert-butyldimethylsilyl)oxy)ethyl)-3-fluoro-4-methyl-8-oxo-5,6,7,8-tetrahydronaphthalen-1-yl)acetamide (1-7d)

To a solution of compound 1-7c (2350 mg, 10 mmol) in THF (40 mL) was added tBuOK (1M in THF) (22 mL, 22 mmol) at 0° C. dropwise and the internal temperature was maintained at 0° C. The mixture was stirred at 0° C. for 30 min, then 1-7b (3432 mg, 12 mmol) was added slowly. LCMS showed the reaction was completed. IN HCl was added to the mixture dropwise to adjust pH to 2. The mixture was poured into saturated NaHCO₃ aq. (50 mL) and was extracted with EtOAc (60 mL*3). The combined organic layer was dried over anhydrous Na₂SO₄, filtered, and concentrated to give the crude product which was purified by silica column gel chromatography (eluent: petroleum ether/EtOAc=100/0 to 83/17) to afford 1-7d (700 mg, 17.8% yield) as a yellow solid.

MS (ESI) m/z: 394.2 [M+H]⁺.

¹H NMR (400 MHz, DMSO-d6) δ=7.40 (s, 2H), 6.34 (d, J=12.6, 1H), 6.34 (d, J=12.6, 1H), 4.45 (t, J=5.2, 1H), 4.45 (t, J=5.2, 1H), 3.65-335 (m, 2H), 3.55-3.45 (m, 2H), 2.83 (s, 1H), 2.83 (s, 1H), 2.72-2.61 (m, 1H), 2.46 (d, 1=5.0, 11H), 2.11-1.99 (m, 3H), 1.97 (d, J=1.0, 3H), 1.72-1.60 (m, 1H), 1.43 (d, J=6.2, 1H).

### Step 3

### 8-amino-6-fluoro-2-(2-hydroxyethyl)-5-methyl-3,4-dihydronaphthalen-1(2H)-one (1-7e)

To a solution of compound 1-7d (700 mg, 1.78 mmol) in MeOH (3 mL) was added 2 N HCl (3 mL). The mixture was stirred at 60° C. for 16 h. LCMS showed the reaction was completed. The mixture was poured into aq. saturated NaHCO₃ (20 mL) and was extracted with EtOAc (30 mL*3). The combined organic layer was dried over anhydrous Na₂SO₄, filtered, and concentrated to give the crude product which was purified by silica column gel chromatography (eluent: petroleum ether/EtOAc=100/0 to 76/24) to afford 1-7e (200 mug, 47% yield) as an off-white solid.

MS (ESI) m/z: 238.2 [M−H]⁺.

### Step 4

### (9S)-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (1-7)

To a solution of compound 1-7e (100 mg, 0.42 mmol) in toluene (6 mL) and o-cresol (0.35 mL) was added 1-4e (110 mg, 0.42 mmol) and PPTS (16 mg, 0.06 mmol). The mixture was refluxed at 140° C. for 5 h. LCMS showed the reaction was completed. Solvent was evaporated and the residue was purified by silica column gel chromatography (eluent: CH₂Cl₂/MeOH=100/0 to 92/8) to afford 1-7 (80 mg, 41% yield) as an orange solid.

MS (ESI) m/z: 465.2 [M+H]⁺.

¹H NMR (400 MHz, DMSO-d6) δ=7.74 (d, 1=11.0, 1H), 7.31 (s, 1H), 6.53 (d, J=1.9, 1H), 5.44 (s, 2H), 5.30 (s, 2H), 4.77 (t, 1=4.8, 1H), 3.69-3.56 (m, 4H), 3.10 (t, J=18.6, 2H), 2.38 (s, 3H), 2.29 (d, J=9.7, 1H), 1.99-1.81 (m, 3H), 1.73 (d, J=5.4, 2H), 0.93-0.81 (m, 3H).

### Example 1-8

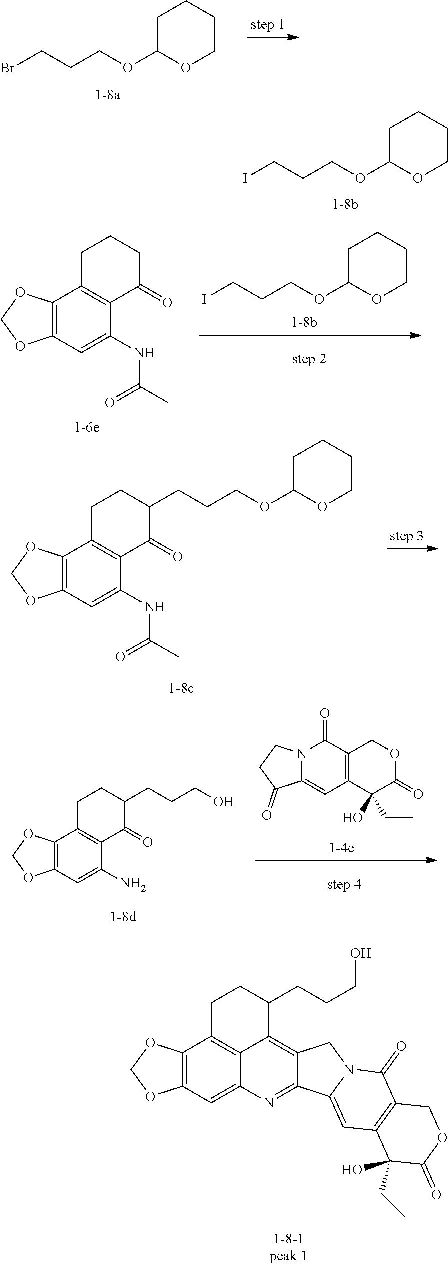

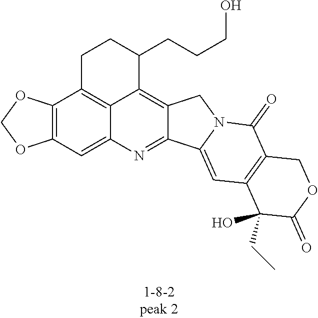 [SMILES: CC[C@@]1(O)C(=O)OCc2c1cc1n(c2=O)Cc2c-1nc1cc3c(c4c1c2C(CCCO)CC4)OCO3]

### Step 1

### 2-(3-iodopropoxy)tetrahydro-2U-pyran (1-8b)

To a solution of compound 1-Sa (2.0 g, 9.01 mmol) in acetone (20 mL) was added NaI (4.0 g, 27 mmol). The mixture was stirred at 60° C. for 3 h. The mixture was diluted with hexane (40 mL) and washed with water (40 mL) and brine (40 mL). The combined organic layer was dried over Na₂SO₄ and concentrated to afford 1-8b (1.3 g, 54% yield) as a colorless oil.

¹H NMR (400 MHz, CDCl₃) δ 4.61 (dd, J=4.4, 2.8 Hz, 1H), 3.91-3.83 (m, 1H), 3.83-3.77 (m, 1H), 3.56-3.49 (m, 1H), 3.45 (dt, J=10.0, 5.9 Hz, 1H), 3.30 (td, J=6.8, 1.0 Hz, 2H), 2.10 (ddd, J=12.7, 6.8, 5.9 Hz, 2H), 1.90-1.63 (m, 3H), 1.61-1.48 (n, 5H).

### Step 2

### N-(6-oxo-7-(3-((tetrahydro-2H-pyran-2-yl)oxy)propyl)-6,7,8,9-tetrahy dronaphtho[1,2-d][1,3]dioxol-5-yl)acetamide (1-8c)

To a solution of compound 1-6e (100 mg, 3.52 mmol) in THF (5 mL) was drop-wised added t-BuOK (1.2 mL, 1.21 mmol, 1 M in THF) at −40° C. under N₂. The mixture was stirred at −40° C. for 30 min under N₂. Then compound 1-8b (164 mg, 0.606 mmol, dissolved in 0.2 mL THF) was drop-wised added to the mixture. The mixture was stirred for 16 h with gradual heating to room-temperature under N₂. The mixture was quenched with sat. NH₄Cl (3 mL) and extracted with EA (3 mL*3). The combined organic layer was dried over anhydrous Na₂SO₄, filtered, and concentrated to give the residue, which was purified by column (petroleum ether/ethyl acetate=1:0 to 4:1) to afford the compound 1-8c (20 mg, 12.7% yield) as a yellow solid.

MS (ESI) m/z: 248.1 [M+H]⁺.

11H NMR (400 MHz, CDCl₃) δ 12.44 (s, 1H), 8.26 (s, 1H), 6.01-5.98 (m, 2H), 4.59-4.53 (m, 1H), 3.90-3.72 (m, 2H), 3.53-3.36 (m, 2H), 3.00-2.88 (m, 1H), 2.79-2.67 (m, 1H), 2.55-2.44 (m, 1H), 2.19 (s, 3H), 2.17-2.09 (n, 1H1), 2.06-1.86 (m, 2H), 1.86-1.65 (m, 6H), 1.60-1.54 (m, 2H).

### Step 3

### 5-amino-7-(3-hydroxypropyl)-8,9-dihydronaphtho[1,2-d][1,3]dioxol-6(7H)-one (1-8d)

To a solution of compound 1-8c (20 mg, 0.05 mmol) in MeOH (5 mL) was added 2 N HCl (5 mL). The mixture was stirred at 60° C. for 3 h. The mixture was concentrated in vacuo to remove MeOH. Then the mixture was adjusted to pH 8 using sat. Na₂CO₃ and extracted with C1H₂Cl₂ (5 mL*3). The combined organic phase was concentrated in vacuo to give the residue, which was purified by silica column gel chromatography (eluent: petroleum ether/ethyl acetate=100/0 to 0/100) to afford 1-8d (10 mg, 74% yield) as a yellow oil.

MS (ESI) m/z: 264.2 [M+H]⁺.

### Step 4

### (1R,1,S)-10-ethyl-10-hydroxy-1-(3-hydroxypropyl)-1,2,3,10,13,16-hexahydro-11H,14H-benzo[de][1,3]dioxolo[4,5-g]pyrano[3′,4′:6,7]indolizino[1,2-b]quinoline-11,14-dione (1-8-1) and (1S,10S)-10-ethyl-10-hydroxy-1-(3-hydroxypropyl)-1,2,3,10,13,16-hexahydro-11H,14H-benzo[de][1,3]dioxolo[4,5-g]pyrano[3′,4′:6,7]indolizino[1,2-b]quinoline-11,14-dione (1-8-2)

To a solution of compound 1-8d (80 mg, 0.304 mmol) in toluene (14 mL) and o-cresol (2 mL) was added 1-4e (88 mg, 0.334 mmol) and PPTS (23 mg, 0.091 mmol). The mixture was refluxed at 140° C. for 16 h. The mixture was concentrated in vacuo to remove toluene and purified by silica column gel chromatography (eluent: Cl₂Cl₂/MeOH=100/0 to 100/10) to give crude product which was further purified by prep-HPLC (FA) (Method: column: XBridge Prep C18 OBD 5 μm 19*150 mm; Mobile phase: A-water (0.1% formic acid): B-acetonitrile; Flow rate: 20 mL/min) The fraction was lyophilized to give 1-8-1 (15 mg, 71% yield) as a white solid and 1-8-2 (20 mg, 13% yield) as a white solid.

Compound 1-8-1 (retention time: 2.54 min).

MS (ESI) m/z: 491.5 [M+H]⁺.

¹H NMR (400 MHz, DMSO-d₆) δ 7.37 (s, 1H), 7.23 (s, 1H), 6.48 (s, 1H), 6.27 (d, J=2.9 Hz, 2H), 5.42 (s, 2H), 5.31 (d, J=18.9 Hz, 1H), 5.19 (d, J=18.8 Hz, 1H), 4.42 (t, J=5.1 Hz, 1H), 3.48-3.42 (m, 2H), 3.01-2.91 (m, 2H), 2.22 (d, J=13.4 Hz, 1H), 1.93-1.75 (m, 4H), 173-1.63 (m, 2H), 1.61-1.54 (m, 2H), 0.87 (t, J=7.3 Hz, 3H).

Compound 1-8-2 (retention time: 2.66 min).

MS (ESI) m/z: 491.5 [M+H]⁺.

¹H NMR (400 MHz, DMSO-d₆) δ 7.37 (s, 1H), 7.23 (s, 1H), 6.49 (s, 1H), 6.27 (s, 2H), 5.42 (s, 2H), 5.31 (d, J:=18.9 Hz, 1H), 5.20 (d, J:=18.9 Hz, 1H), 4.43 (t, J=5.2 Hz, 1H), 3.47-3.43 (m, 2H), 3.02-2.86 (m, 2H), 2.22 (d, J=13.3 Hz, 1H), 1.94-1.78 (m, 3H), 1.73-1.63 (m, 2H), 1.63-1.53 (m, 3H), 0.87 (t, J=7.3 Hz, 3H).

### Example 1-9

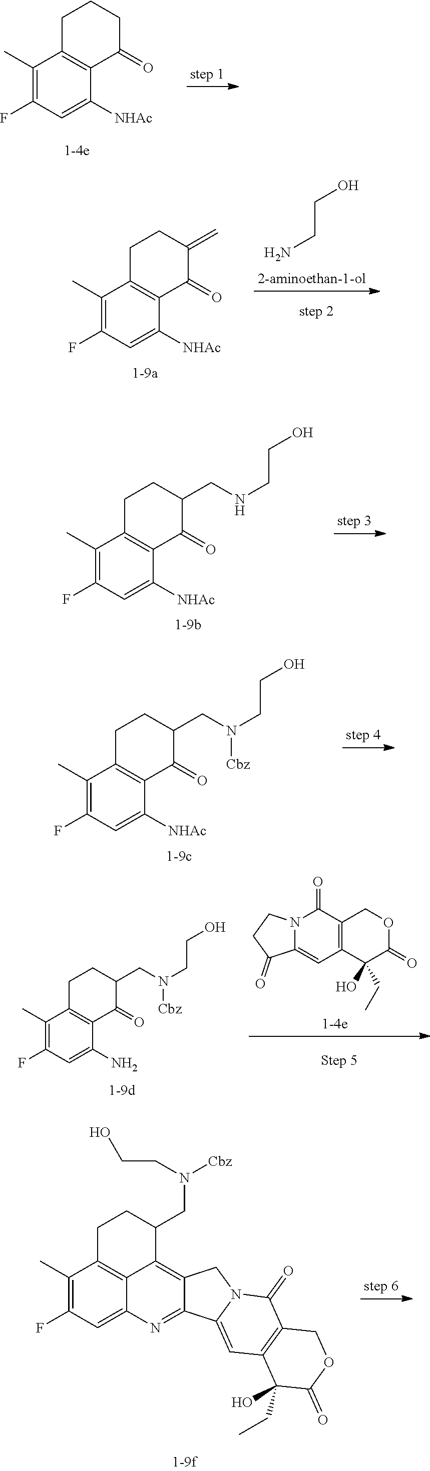

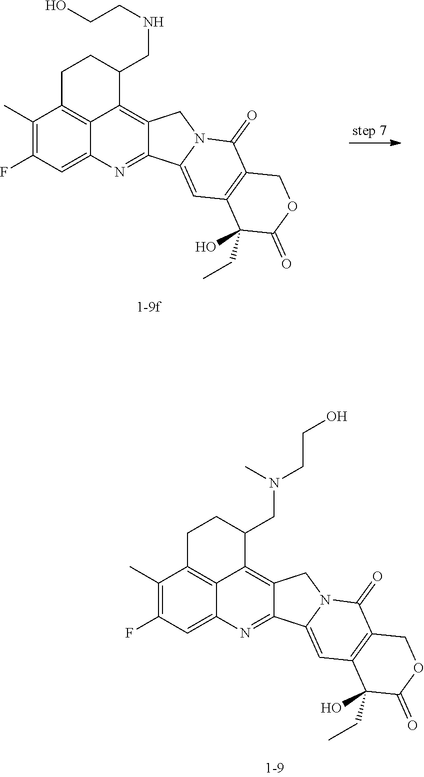 [SMILES: CC[C@@]1(O)C(=O)OCc2c1cc1n(c2=O)Cc2c-1nc1cc(F)c(C)c3c1c2C(CNCCO)CC3 ; CC[C@@]1(O)C(=O)OCc2c1cc1n(c2=O)Cc2c-1nc1cc(F)c(C)c3c1c2C(CN(C)CCO)CC3]

### Step 1

### N-(3-fluoro-4-methyl-7-methylene-8-oxo-5,6,7,8-tetrahydronaphthalen-1-yl)acetamide (1-9a)

To a solution of 1-4a (470.5 mg, 2 mmol) in CH₃CN (5 mL) was added paraformaldehyde (150 mg, 5 mmol) and ZnCl₂ (136.3 mg, 1 mmol). The mixture was stirred at r.t. for 10 min. After that pyrrolidine (287 mg, 4 mmol) was added dropwise. Upon completion of addition, the mixture was stirred at 70° C. for 2 h. Then the reaction solution was cooled to r.t., the solvent was removed by evaporation and the residue was directly purified by silica column gel chromatography (eluent: hexane/EtOAc=30/I to 5/1) to afford 1-9a as a white solid (210 mg, yield 42.5%).

¹H NMR (400 MHz, CDCl₃) δ 12.42 (s, 1H), 8.46 (d, J=12.9 Hz, 1H), 6.19 (d, J=13 Hz, 1H), 5.51 (d, J=1.6 Hz, 1H) 2.93 (t, J=6.5 Hz, 2H), 2.78 (t, J=6.4 Hz, 2H), 2.25 (s, 3H), 2.17 (d, J=1.9 Hz, 3H).

MS (ESI) m/z: 248.2 [M+H]⁺.

### Step 2

### N-(3-fluoro-7-(((2-hydroxyethyl)amino)methyl)-4-methyl-8-oxo-5,6,7,8-tetrahydronaphthalen-1-yl)acetamide (1-9b)

To a solution of 1-9a (200 mg, 0.8 mmol) in THE (10 mL) was added 2-aminoethan-1-ol (108.8 mg, 1.78 mmol). The mixture was stirred at r.t. for 40 min. Then the solvent was removed by evaporation. The crude product, which is not stable under silica gel purification, was used directly in the next step without further purification (290 mg, crude).

MS (ESI) m/z: 309.3 [M+H]⁺.

### Step 3

### N-(3-fluoro-7-(((2-hydroxyethyl)amino)methyl)-4-methyl-8-oxo-5,6,7,8-tetrahydronaphthalen-1-yl)acetamide (1-9c)

To a solution of 1-9b (80 mg, 0.26 mmol) in CH₂Cl₂ (2 mL) was added Et₃N (40 mg, 0.4 mmol). The solution was then cooled to 0° C. and benzyl chloroformate (48.6 mg, 0.28 mmol) was then added dropwise. The mixture was then warmed to r.t. and stirred at r.t. for 2 h. The solvent was removed by evaporation and the residue was directly purified by silica column gel chromatography (eluent: CH₂C₂/MeOH=32/1) to afford 1-9c as a colorless oil (110 mg, yield 95.6%).

¹H NMR (400 MHz, CDCl₃) δ 12.09 (s, 1H), 8.39 (d, J=13.0 Hz, 1H), 7.42-7.27 (m, 4H), 7.21 (s, 1H), 5.30 (s, 1H), 5.13 (d, J=13.1 Hz, 2H), 4.94 (d, J=12.0 Hz, 1H), 3.99-3.71 (m, 3H), 3.65-3.53 (m, 1H), 3.52-3.35 (m, 2H), 2.85 (t, J=62.3 Hz, 3H), 2.21 (s, 3H), 2.10 (d, J=27.8 Hz, 3H), 1.79 (s, 2H).

MS (ESI) m/z: 465.4 [M+Na]⁺.

### Step 4

### benzyl ((8-amino-6-fluoro-5-methyl-1-oxo-1,2,3,4-tetrahydronaphthalen-2-yl)methyl)(2-hydroxyethyl)carbamate (1-9d)

To a solution of compound 1-9c (100 mg, 0.23 mmol) in MeOH (3 mL) was added HCl (2 N, 1.5 mL). The mixture was stirred at 60° C. for 3 h. The mixture was cooled to r.t. and adjusted to pH 8 using sat. NaHCO₃. The mixture was extracted with EtOAc (10 mL*3). The organic layer was dried and concentrated. The crude product 1-9d (red oil) was used directly in the next step without further purification (crude: 81 mg, yield 89.6%).

MS (ESI) m/z: 401.4 [M+H]⁺.

### Step 5

Nbenzyl (((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)methyl)(2-hydroxyethyl)carbamate (1-9e)

To a solution of 1-9d (80 mg, 0.2 mmol) in toluene (10 mL) and o-cresol (0.5 mL) was added 1-4e (60.5 mg, 0.23 mmol) and PPTS (30.2 mg, 0.12 mmol). The mixture was refluxed at 140° C. for 12 h. The solvent was removed by evaporation and the crude product 1-9e was used directly in the next step without further purification (crude: 120 mg, yield 95.6%).

MS (ESI) n/z: 628.5 [M+H]⁺.

### Step 6

### Nbenzyl (9S)-9-ethyl-5-fluoro-9-hydroxy-1-(((2-hydroxyethyl)amino)methyl)-4-methyl-1,2,3,9,12,15-hexahydro-10H,13H-benzo[de]pyrano[3′,4′:6,7]indolizino[1,2-b]quinoline-10,13-dione (1-9f)

To the solution of 1-9e (crude 120 mg, 0.2 mmol) in MeOH (2 mL) and THF (2 mL) was added wet Pd/C (20%, 24 mg), stirred under H₂ atmosphere at r.t. for 8 h. The solution was filtered through celite and concentrated under vacuum. The residue was purified by prep-HPLC (FA) (Method: column: XBridge Prep C18 OBD 5 am 19*150 mm; Mobile phase: A-water (0.1% FA): B-acetonitrile; Flow rate: 20 mL/min). The fraction was lyophilized to give 1-9f as a white solid (33.2 mg, 33.6% yield).

¹H NMR (400 MHz, DMSO-d) δ 8.21 (s, 1H), 7.74 (d, J=11.0 Hz, 1H), 7.30 (s, 1H), 6.53 (s, 1H), 5.44 (s, 2H), 5.41-5.34 (m, 2H), 5.31 (s, 1H), 3.55 (s, 1H), 3.49 (dd, J=5.6, 3.8 Hz, 2H), 3.05 (dd, J=27.9, 9.3 Hz, 3H), 2.89-2.81 (m, 2H), 2.74-2.67 (m, 2H), 2.37 (s, 3H), 2.03-1.81 (m, 4H), 0.87 (dd, J=11.7, 4.3 Hz, 3H).

MS (ESI) m/z: 494.4 [M+H]⁺.

### Step 7

### (9S)-9-ethyl-5-fluoro-9-hydroxy-1-(((2-hydroxyethyl)(methyl)amino)methyl)-4-methyl-1,2,3,9,12,15-hexahydro-1011,1311-benzo[de]pyrano[3′,4′:6,7]indolizino[1,2-b]quinoline-10,13-dione (1-9)

To a solution of 1-9f (10 mg, 0.02 mmol) in MeOH (1 mL) was added 37% formaldehyde solution (2.8 μL, 0.04 mmol). The solution was then cooled to 0° C. and NaBH₃CN (2 mg, 0.03 mmol) was then added in one portion. The solution was then warmed to r.t. and stirred for another 2 h. The mixture was then quenched with H₂O and purified by prep-HPLC (FA) (Method: column: XBridge Prep C18 OBD 5 μm 19*150 mm; Mobile phase: A-water (0.1% FA): B-acetonitrile; Flow rate: 20 mL/min). The fraction was lyophilized to give 1-9 as a white solid (5.5 mg, 54.2% yield).

MS (ESI) m/z: 508.5 [M+H]⁺.

General procedures for preparation of linker-payloads.

General procedure A: preparation of Example 2-1.

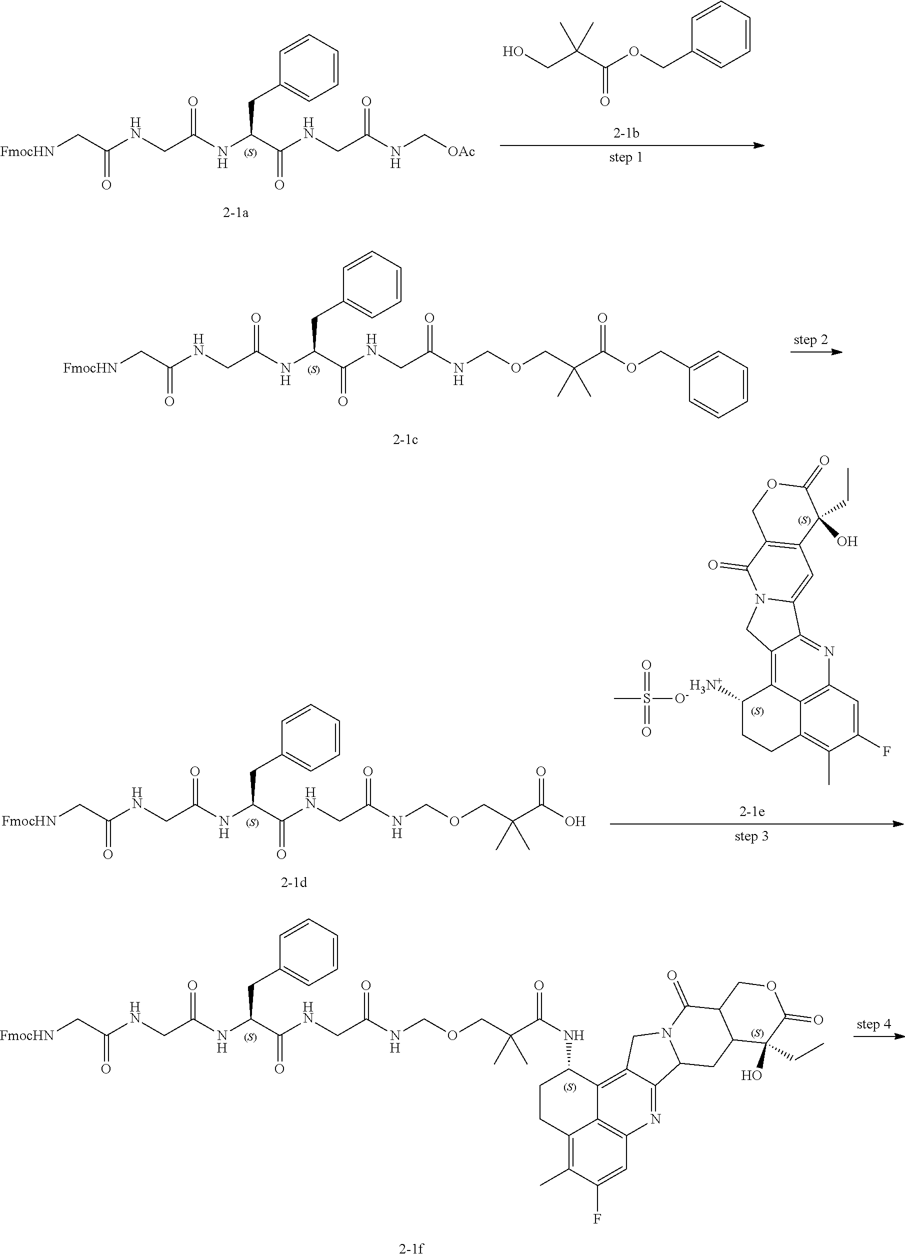

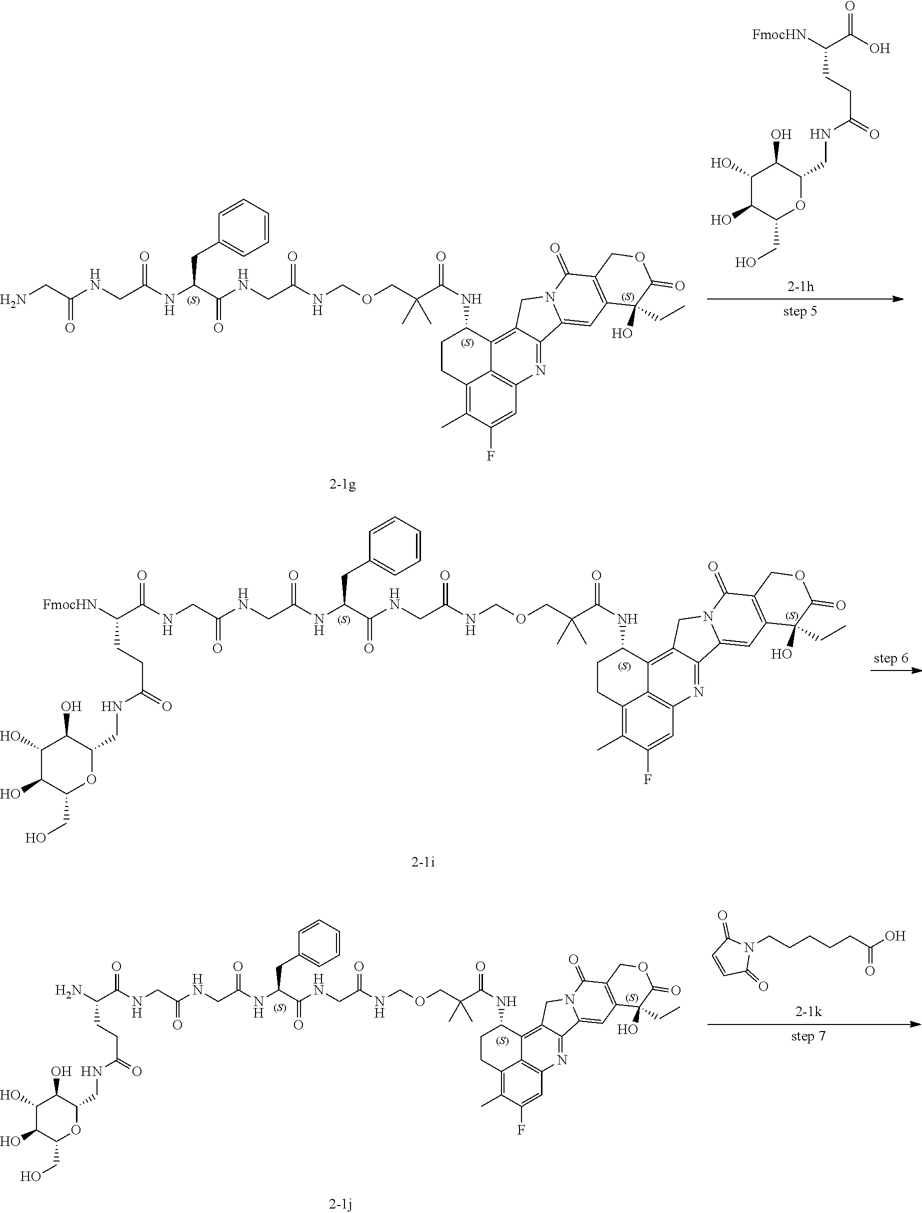

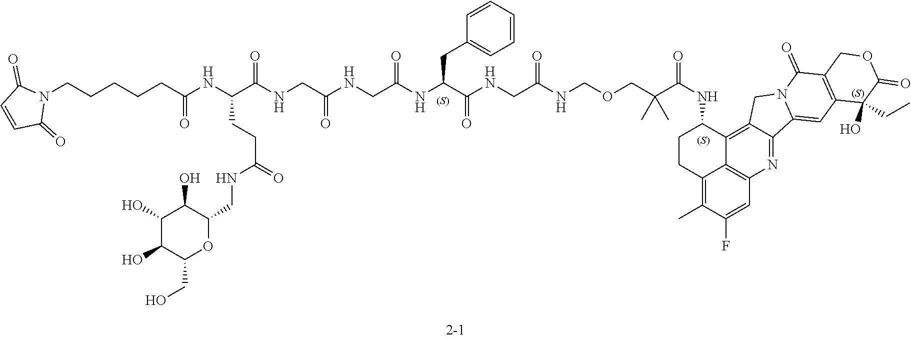

### Step 1

### Benzyl(S)-11-benzyl-1-(9H-fluoren-9-yl)-20,20-dimethyl-3,6,9,12,15-pentaoxo-2,18-dioxa-4,7,10,13,16-pentaazahenicosan-21-oate (2-1c)

To solution of 2-1a (250 mg, 0.40 mmol) and 2-1b (83 mg, 0.40 mmol) in THF (5 mL) was added 4 Å molecular sieve. The mixture was stirred at r.t. for 10 min then Sc(OTf)₃ (195 mg, 0.40 mmol) was added and reacted at r.t. for another 16 h. The suspension mixture was filtered through a pad of celite, and the cake was washed with THF (10 mL), then the filtrate was quenched by addition of sat. NaHCO₃ (10 mL), extracted with EtOAc (30 mL*2). After separation the combined organic layers were washed with brine (50 mL), dried over Na₂SO₄, filtered and the filtrate was concentrated under vacuum to give a residue. It was purified by silica gel column chromatography (A-DCM; B-MeOH, MeOH/DCM=0/100 to 95/5) to provide 2-1c (90 mg, 29.2% yield).

MS (ESI) m/z: 800.5 [M+Na]⁺.

### Step 2

### (S)-11-benzyl-1-(9H-fluoren-9-yl)-20,20-dimethyl-3,6,9,12,15-pentaoxo-2,18-dioxa-4,7,10,13,16-pentaazahenicosan-21-oic acid (2-1d)

To a solution of 2-1c (80 mg, 0.10 mmol) in MeOH (3 mL) was added wet Pd/C (20 mg). The black suspension was purged with H₂ balloon for three times then reacted at r.t. for 2 h under H2 balloon. After the reaction was completed, the black suspension was filtered off through a pad of celite and the cake was washed with MeOH. The combined organic layers were concentrated under vacuum to provide 2-Id (61 mg, 84.8% yield).

MS (ESI) m/Z: 710.4 [M+Na]⁺.

### Step 3

### (9H-fluoren-9-yl)methyl ((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)carbamate (2-1f)

To a mixture of 2-Id (60 mg, 0.087 mmol) and HATU (33 mg, 0.087 mmol) in DMF (2 mL) was added DIPEA (43 μL, 34 mg, 0.26 mmol). The mixture was reacted at r.t. for 10 min. 2-1e (46 mg, 0.087 mmol) was added and reacted at the same temperature for another 1 h. After the reaction was completed, the mixture was filtered and the filtrate was purified using prep-HPLC (Method; column: XBridge Prep C18 OBD 5 μm 19*150 mm; Mobile phase: A-water (0.1% formic acid): B-acetonitrile; Flow rate: 20 mL/min) to provide 2-If (85 mg, 88.2% yield).

MS (ESI) m/z: 1105.5 [M+H]⁺.

### Step 4

### 3-(((S)-13-amino-7-benzyl-3,6,9,12-tetraoxo-2,5,8,11-tetraazatridecyl)oxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,2-dimethylpropanamide (2-1 g)

To a solution of 2-If (85 mg, 0.062 mmol) in DMF (2 mL) was added Et₂NH (64 μL, 46 mg, 0.62 mmol). The mixture was stirred at r.t. for 0.5 h. On completion of the reaction, the mixture was concentrated under vacuum to give 2-Ig (86 mg, crude) as a yellow solid.

MS (ESI) m/z: 883.5 [M+H]⁺.

### Step 6

### (9H-fluoren-9-yl)methyl ((6S,15S)-15-benzyl-25-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-24,24-dimethyl-3,7,10,13,16,19,25-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (2-1i)

To a solution of 2-Ig (86 mg, crude) and 2-1h (43 mg, 0.079 mmol) in DMF (1.5 mL) was added DIPEA (26 μL, 21 mg, 0.16 mmol). The mixture was stirred at r.t. for 1.5 h. On completion of the reaction, the mixture was purified by prep-HPLC (FA) (Method: column: XB3ridge Prep C18 OBD 5 μm 19*150 mm; Mobile phase: A-water (0.1% formic acid): B-acetonitrile; Flow rate: 20 mL/min). The fraction was lyophilized to give 2-1i (70 mg, 62.6% yield) as a white powder.

MS (ESI) m/z: 1410.7 [M+H]⁺.

### Step 7

### (S)-2-amino-N1-((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (2-1j)

To a solution of 2-li (70 mg, 0.050 mmol) in DIF (1 mL) was added Et₂NH (51 μL, 36 mg, 0.50 mmol). The mixture was stirred at r.t. for 0.5 h. On completion of the reaction, the mixture was concentrated under vacuum to give 2-1j (71 mg, crude) as a yellow solid.

MS (ESI) m/z: 1188.2 [M+H]⁺.

### Step 8

### (S)—N¹—((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N⁵-(((2S,3R,4R,5S,6R)-3,4,5-trihydroxy-6-(hydroxymethyl)tetrahydro-2H-pyran-2-yl)methyl)pentanediamide (2-1)

To a solution of 2-1k (19 mg) in DMF (3 mL) were added HATU (34 mg, 0,088 mmol) and DIPEA (10 μL, 7.6 mg, 0.059 mmol). The resulting yellow solution was stirred at r.t. for 5 min then 2-1j (71 mg, crude) was added. The mixture was stirred at r.t. for 60 min. On completion of the reaction, the mixture was purified by prep-HPLC (FA) (Method: column:XBridge Prep C18 OBD 5 μm 19*150 mm; Mobile phase: A-water (0.1% formic acid): B-acetonitrile; Flow rate: 20 mL/min). The fraction was lyophilized to give 2-1 (32 mg, 26.3% yield) as a white powder.

MS (ESI) m/z: 1381.1 [M+H]⁺.

General procedure B: preparation of Example 2-4.

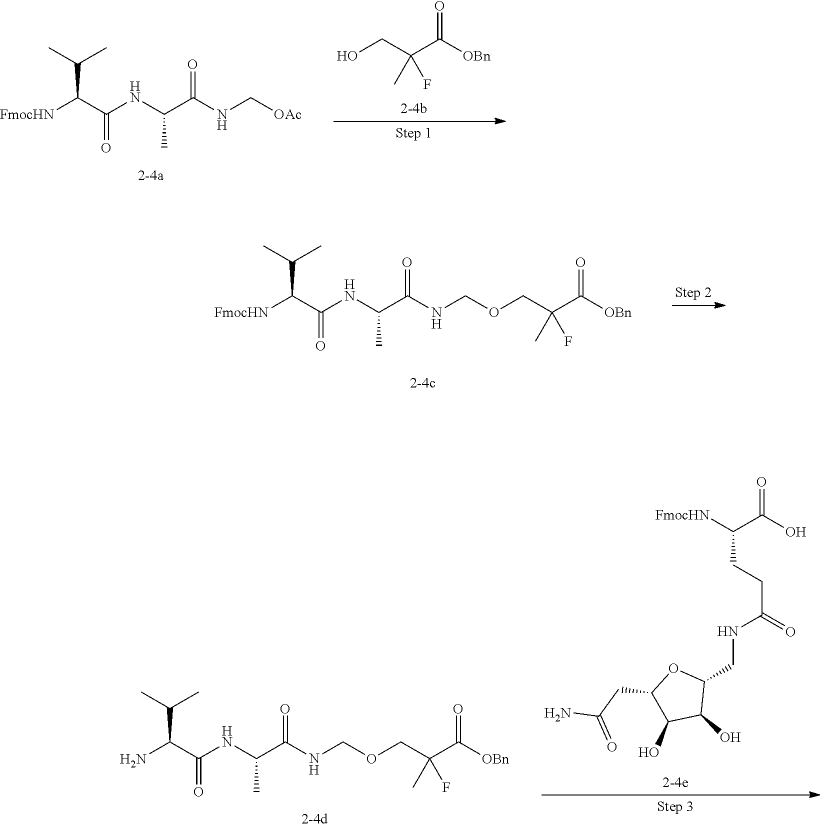

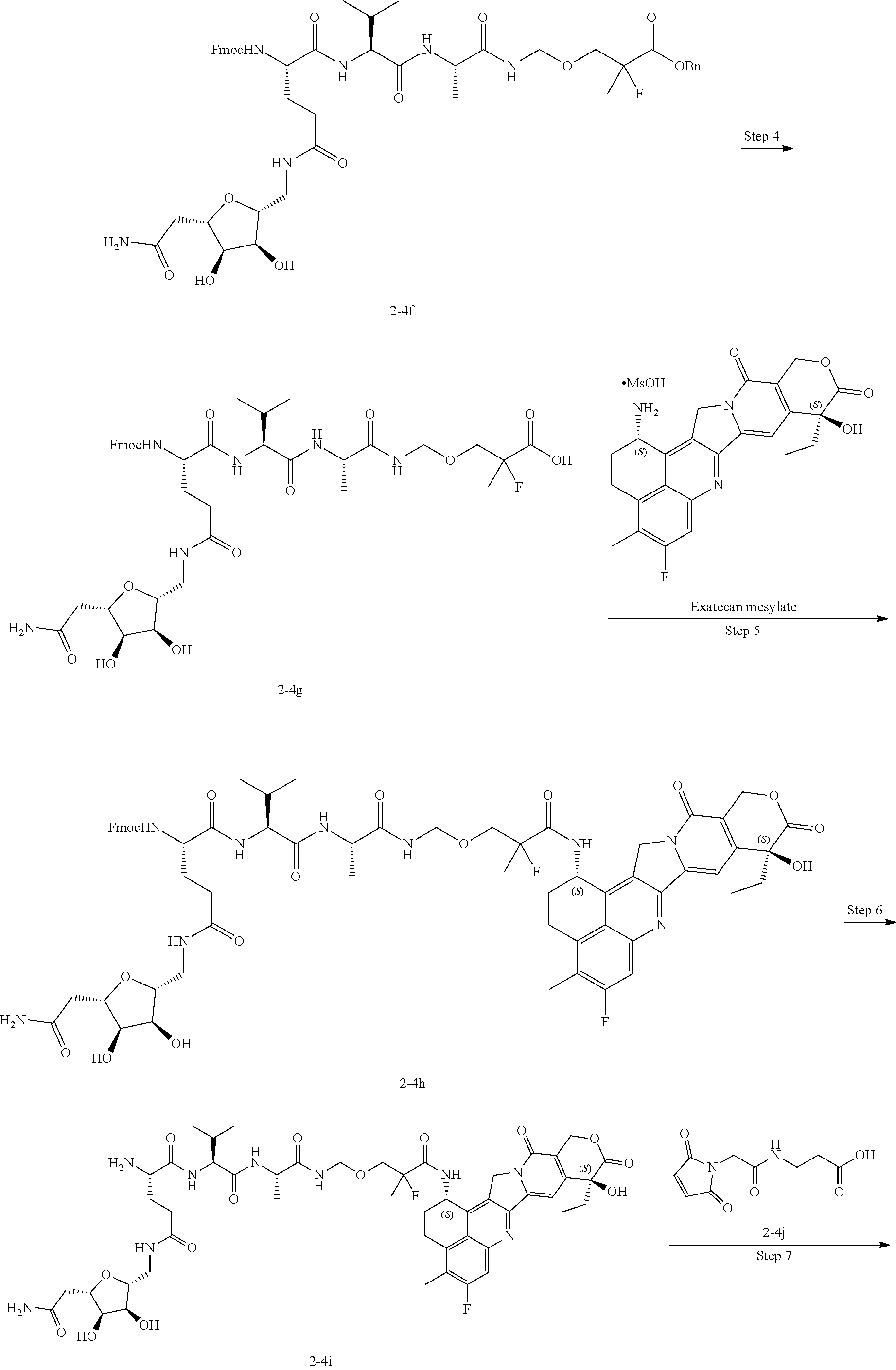

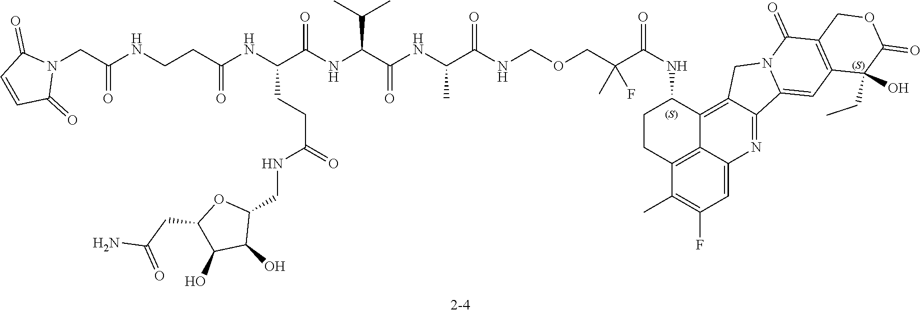

### Step 1

### benzyl (5S,8S,14R)-1-(9H-fluoren-9-yl)-14-fluoro-5-isopropyl-8,14-dimethyl-3,6,9-trioxo-2,12-dioxa-4,7,10-triazapentadecane-15-oate (2-4c)

To a mixture of 2-4a (3.0 g, 6.23 mmol), 2-4b (1.98 g, 9.34 mmol), and freshly dried 4 Å molecular sieves (6 g) in dry THE (30 mL) was added scandium trifluoromethanesulfonate (3.99 g, 8.10 mmol), stirred at r.t. under N₂ atmosphere overnight. The solution was filtered through celite, diluted with EtOAc (300 mL), and washed by saturated NaHCO₃ (50 mL*3). The organic layer was dried over anhydrous Na₂SO₄, filtered and concentrated. The residue was purified by silica column gel chromatography (eluent: petroleum ether/EtOAc=100/0 to 50/50) to afford compound 2-4c (3.5 mg, 84% yield).

MS (ESI) m/z: 656.5 [M+Na]⁺.

### Step 2

### benzyl (R)-3-(((S)-2-(S)-2-amino-3-methylbutanamido)propanamido)methoxy)-2-fluoro-2-methylpropanoate (2-4d)

To a solution of compound 2-4c (3.5 g, 5.52 mmol) in DMF (30 mL) was added Et₂NH (4.04 g, 55.2 mmol). The mixture was stirred at r.t. for 30 min. The mixture was concentrated under high vacuum to give compound 2-4d (2.2 g, crude) as an off-white solid, which was used directly without further purification.

MS (ESI) m/z: 412.4 [M+H]Q.

### Step 3

### benzyl (5S,8S,11S,17R)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-17-fluoro-8-isopropyl-11,17-dimethyl-3,6,9,12-tetraoxo-2,15-dioxa-4,7,10,13-tetraazaoctadecan-18-oate (2-41)

To a solution of compound 2-4d (2.2 g, 5.35 mmol) in DMF (20 mL) were added compound 2-4e (3.04 g, 5.61 mmol), HATU (3.05 g, 8.02 mmol), and DIEA (138 g, 10.69 mmol). The mixture was stirred at r.t. for 30 min. The mixture was poured into CH₃CN (50 mL), stirred for 30 min, the solid was filtered and further purified by trituration with CH₃CN:H₂O (50 mL, 10:1 v:v) to give compound 2-4f (4.1 g, 82% yield) as a pale-white solid.

MS (ESI) m/z: 957.8 [M+Na]⁺.

### Step 4

### (5S,8S,11S,17R)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-17-fluoro-8-isopropyl-11,17-dimethyl-3,6,9,12-tetraoxo-2,15-dioxa-4,7,10,13-tetraazaoctadecan-18-oic acid (2-4g)

To a solution of compound 2-4f (3.0 g, 3.21 mmol) in co-solvent DMF-MeOH (40 mL, 1:1, v:v) was added Pd/C (10%, 300 mg). The mixture was stirred at 1-2 atmosphere (15 psi) for 7 h. The mixture was filtered through a pad of celite, concentrated to give compound 2-4g (2.5 mg, crude) as a white solid.

MS (ESI) m/z: 867.7 [M+Na]⁺.

### Step 5

### (9H-fluoren-9-yl)methyl ((6S,9S,12S,18R)-1-((2R,3S,4R,5S)-5-(2-amino-2-oxoethyl)-3,4-dihydroxytetrahydrofuran-2-yl)-19-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-18-fluoro-9-isopropyl-12,18-dimethyl-3,7,10,13,19-pentaoxo-16-oxa-2,8,11,14-tetraazanonadecane-6-yl)carbamate (2-4h)

To a solution of compound exatecan mesylate (1000 mg, 1.18 mmol) in DMF (20 mL) were added compound 2-4g (692 mg, 1.3 mmol), HATU (675 mg, 1.78 mmol), and DIEA (459 mg, 3.55 mmol). The mixture was stirred at r.t. for 30 min. The mixture was concentrated and purified by a silica gel column chromatography (eluent: DCM/MeOH-100/0 to 20/80) to give the title compound 2-4h (1320 mg, 88.6% yield) as an off-white solid.

MS (ESI) m/z: 1285.0 [M+Na].

### Step 6

### (S)-2-amino-N5-(((2R,3S,4R,5S)-5-(2-amino-2-oxoethyl)-3,4-dihydroxytetrahydrofuran-2-yl)methyl)-N1-((S)-1-(((S)-1-((((R)-3-(((I S,9S)-9-ethyl-5-fluoro-9-hydroxy-4-methyl-10,13-dioxo-2,3,9,10,13,15-hexahydro-1H1,12-H-benzo[de]pyrano[3′,4′:6,7]indolizino[1,2-b]quinolin-1-yl)amino)-2-fluoro-2-methyl-3-oxopropoxy)methyl)amino)-1-oxopropan-2-yl)amino)-3-methyl-1-oxobutan-2-yl)pentanediamide (2-4i)

To a solution of compound 2-4h (1000 mg, 0.793 mmol) in DMF (20 mL) was added Et₂NH (580 mg, 7.93 mmol). The mixture was stirred at r.t. for 30 min. The mixture was concentrated under high vacuum to give compound 2-4i (824.6 mg, crude) as an off-white solid, which was used directly without further purification.

MS (ESI) m/z: 1040.9 [M+H11].

### Step 7

### (S)—N5-(((2R,3S,4R,5S)-5-(2-amino-2-oxoethyl)-3,4-dihydroxytetrahydrofuran-2-yl)methyl)-2-(3-(2-(2,5-dioxo-2,5-dihydro-1H-pyrrol-1-yl)acetamido)propanamido)-N1-((S)-1-(((S)-1-((((R)-3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-fluoro-2-methyl-3-oxopropoxy)methyl)amino)-1-oxopropan-2-yl)amino)-3-methyl-1-oxobutan-2-yl)pentanediamide (2-4)

To a solution of compound 2-4i (824 mg, 0.792 mmol) in DMF (15 mL) were added compound 2-4j (233.0 mg, 1.03 mmol), HATU (391.6 mg, 1.03 mmol), and DIEA (204.8 mg, 1.58 mmol). The mixture was stirred at r.t. for 30 min. The mixture was purified by prep-HPLC (FA) (Method: column: XBridge Prep C18 OBD 5 μm 19*150 mm; Mobile phase: A-water (0.1% formic acid): B-acetonitrile; Flow rate: 20 mL/min). The fraction was lyophilized to give compound 2-4 (375 mg, 37.9% yield) as a white solid.

MS (ESI) m/z: 1271.0 [M+Na].

General procedure C: preparation of Example 2-7.

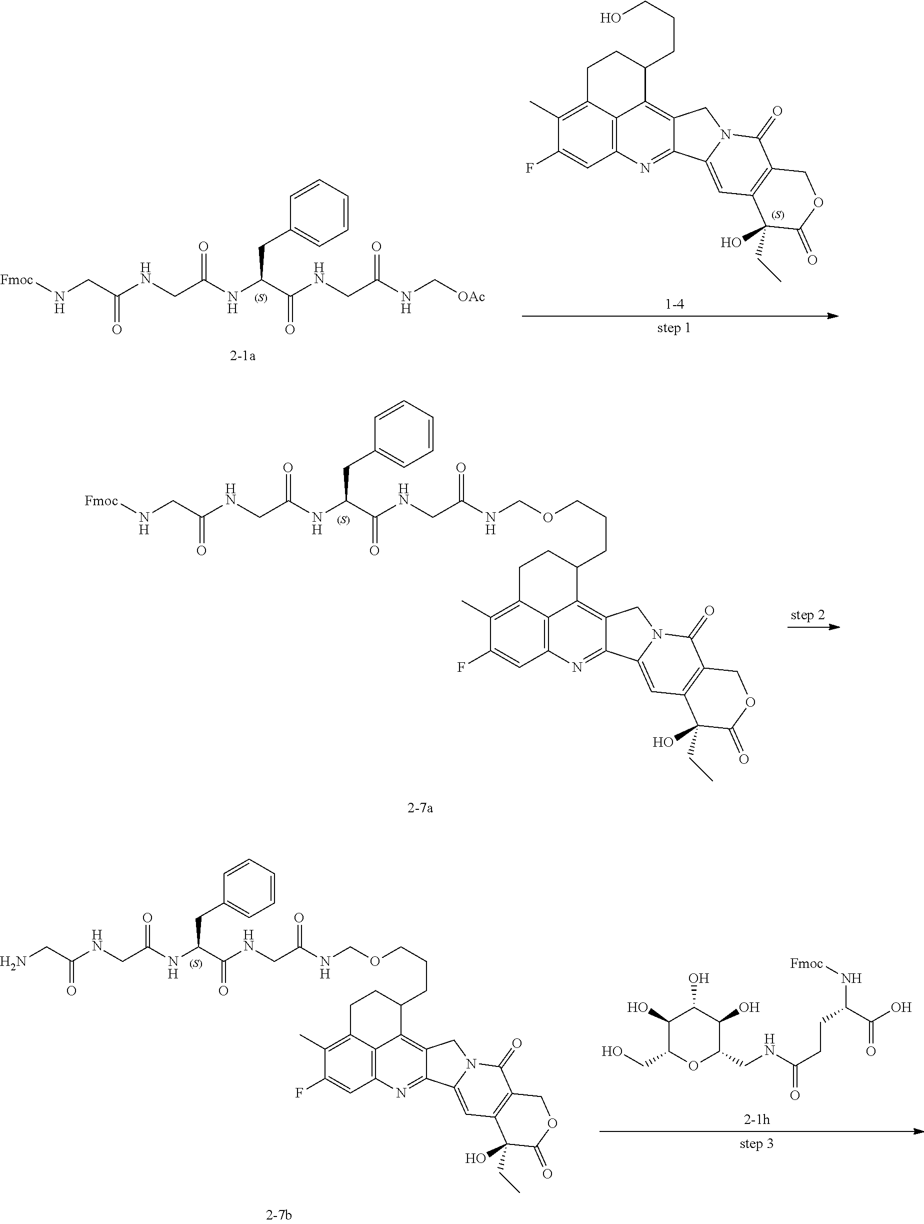

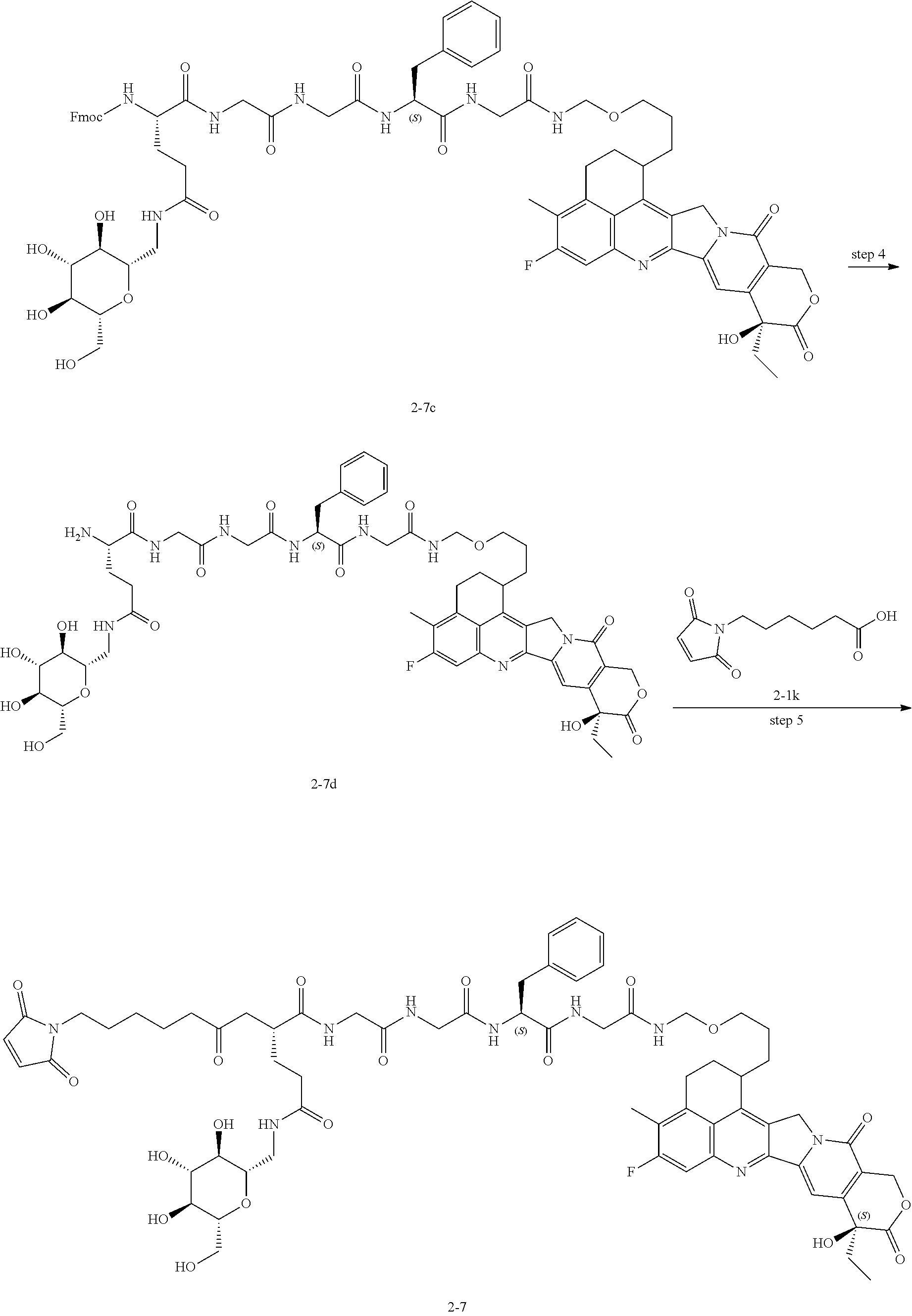

### Step 1

### (9H-fluoren-9-yl)methyl ((7S)-7-benzyl-17-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,5,8,11-tetraoxo-14-oxa-3,6,9,12-tetraazaheptadecyl)carbamate (2-7a)

Compound 2-7a(103 mg, 97% purity) was obtained according to the procedure described in Step 1 of Example 2-L

MS (ESI) m/z: 1048.6 [M+H]⁺.

### Step 2

### (2S)-2-(2-(2-aminoacetamido)acetamido)-N-(2-(((3-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)propoxy)methyl)amino)-2-oxoethyl)-3-phenylpropanamide (2-7b)

Compound 2-7b (105 mg, crude) was obtained according to the procedure described in Step 4 of Example 2-1.

MS (ESI) m/z: 826.5 [M+H]⁺.

### Step 3

### (9H-fluoren-9-yl)methyl ((6S,15S)-15-benzyl-25-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3,7,10,13,16,19-hexaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (2-7c)

Compound 2-7c (50 mg, 98% purity) was obtained according to the procedure described in Step 5 of Example 2-1.

MS (ESI) m/z: 1352.8 [M+H]⁺.

### Step 4

### (2S)-2-amino-N¹-((7S)-7-benzyl-17-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,5,8,11-tetraoxo-14-oxa-3,6,9,12-tetraazaheptadecyl)-N-(((2S,3R,4R,5S,6R)-3,4,5-trihydroxy-6-(hydroxymethyl)tetrahydro-2H-pyran-2-yl)methyl)pentanediamide (2-7d)

Compound 2-7d (52 mg, crude) was obtained according to the procedure described in Step 6 of Example 2-1.

MS (ESI) m/z: 1130.7 [M+H]⁺.

### Step 5

### (2S)—N¹-((7S)-7-benzyl-17-((9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,5,8,11-tetraoxo-14-oxa-3,6,9,12-tetraazaheptadecyl)-2-(6-(2,5-dioxo-2,5-dihydro-1H-pyrrol-1-yl)hexanamido)-N⁵-(((2S,3R,4R,5S,6R)-3,4,5-trihydroxy-6-(hydroxymethyl)tetrahydro-2-J-pyran-2-yl)methyl)pentanediamide (2-7)

Compound 2-7 (33 mg, 98% purity) was obtained according to the procedure described in Step 7 of Example 2-1.

MS (ESI) m/z: 1323.8 [M+H]⁺.

General procedure D: preparation of Example 2-8.

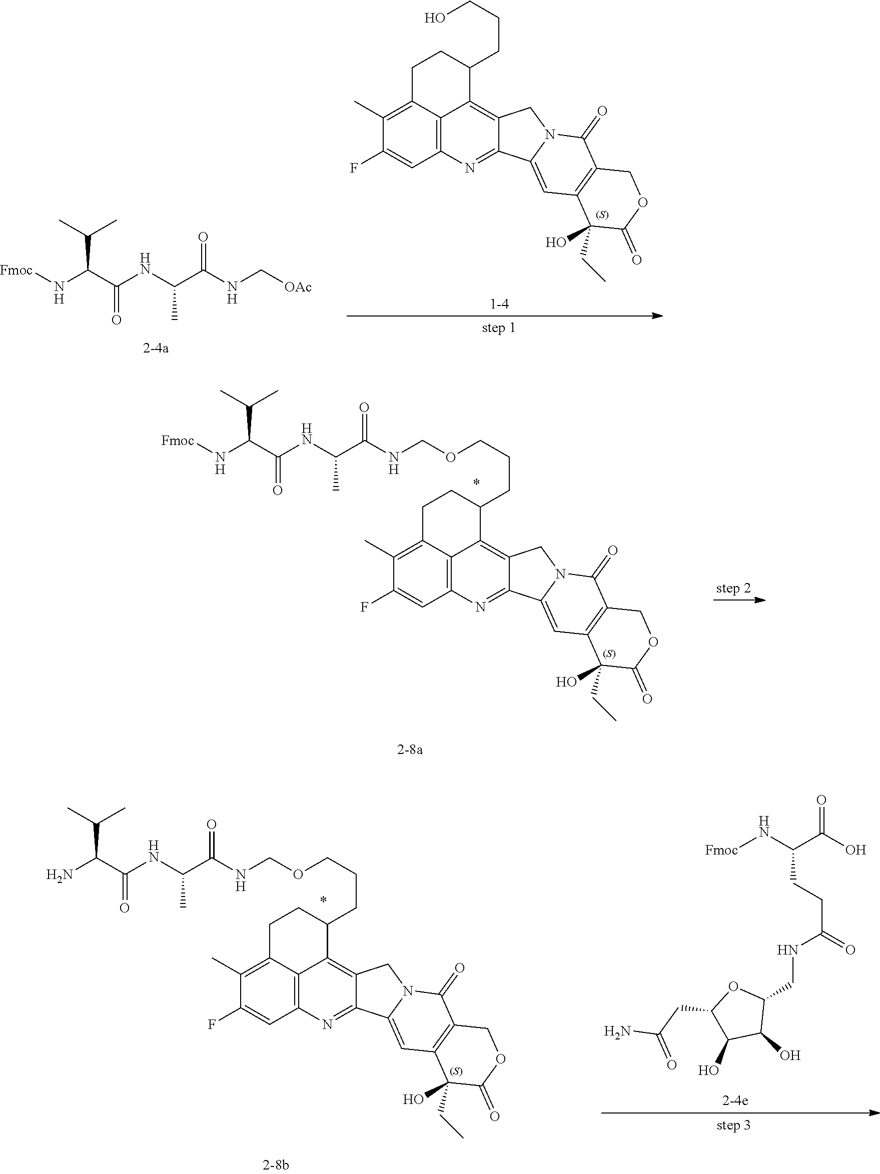

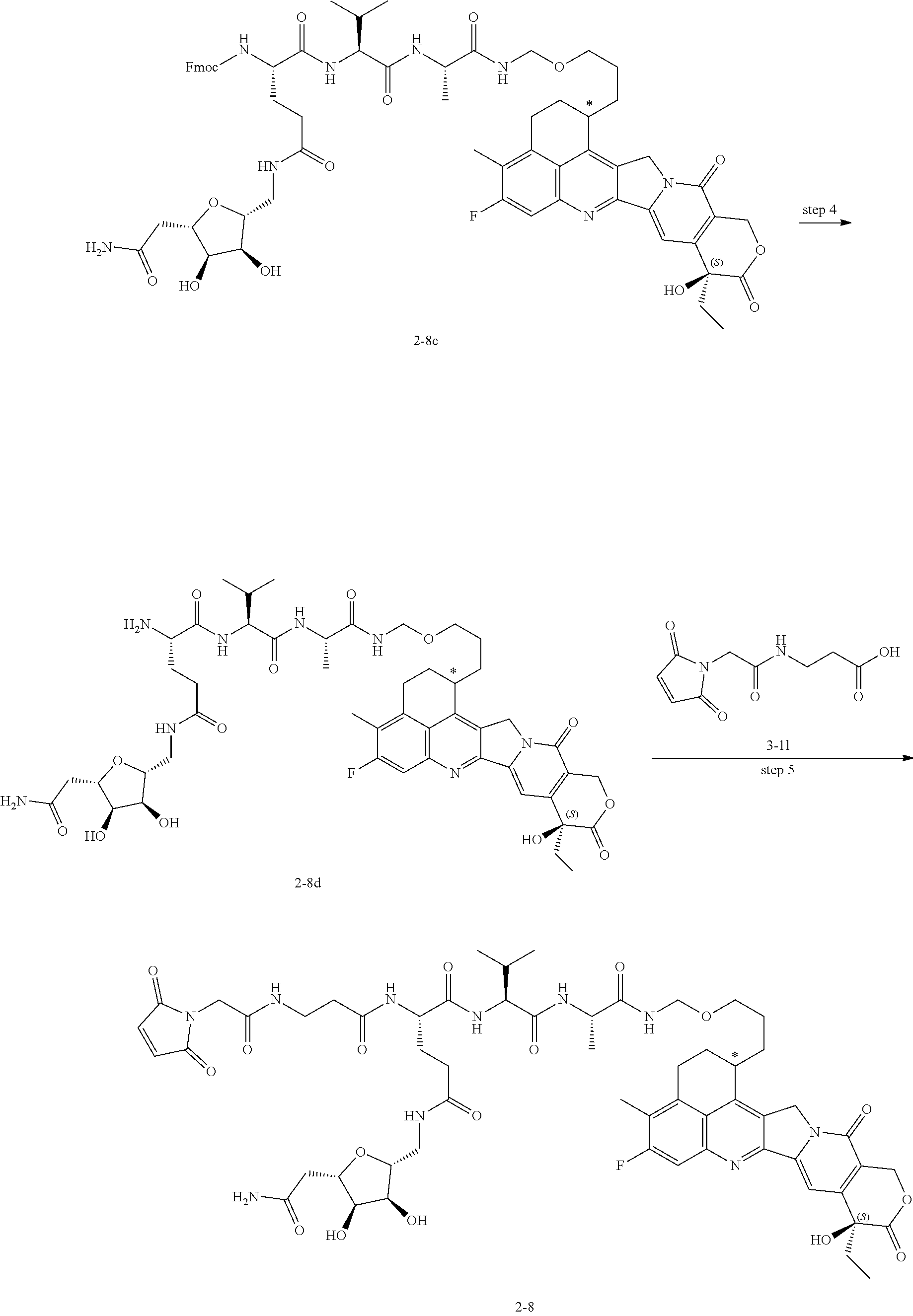

### Step 1

### (9H-fluoren-9-yl)methyl ((S)-1-(((S)-1-(((3-((S)-9-ethyl-5-fluoro-9-hydroxy-4-methyl-10,13-dioxo-2,3,9,10,13,15-hexahydro-1H,12H-113-benzo[de]pyrano[3′,4′:6,7]indolizino[1,2-b]quinolin-1-yl)propoxy)methyl)amino)-1-oxopropan-2-yl)amino)-3-methyl-1-oxobutan-2-yl)carbamate (2-8a)

Compound 2-8a (81 mg, 91% purity) was obtained according to the procedure described in Step 1 of Example 2-4.

MS (ESI) m/z: 900.8[M+H]⁺.

### Step 2

### (S)-2-amino-N—((S)-1-(((3-((S)-9-ethyl-5-fluoro-9-hydroxy-4-methyl-10,13-dioxo-2,3,9,10,13,15-hexahydro-1H,12H-113-benzo[de]pyrano[3′,4′:6,7]indolizino[1,2-b]quinolin-1-yl)propoxy)methyl)amino)-1-oxopropan-2-yl)-3-methylbutanamide (2-8b)

Compound 2-8b (83 mg, crude) was obtained according to the procedure described in Step 4 of Example 2-4.

MS (ESI) m/z: 678.7 [M+H]⁺.

### Step 3

### (9H-fluoren-9-yl)methyl ((6S,9S,12S)-1-((2R,3S,4R,5S)-5-(2-amino-2-oxoethyl)-3,4-dihydroxytetrahydrofuran-2-yl)-19-((S)-9-ethyl-5-fluoro-9-hydroxy-4-methyl-10,13-dioxo-2,3,9,10,13,15-hexahydro-1H,12H-113-benzo[de]pyrano[3′,4′:6,7]indolizino[1,2-b]quinolin-1-yl)-9-isopropyl-12-methyl-3,7,10,13-tetraoxo-16-oxa-2,8,11,14-tetraazanonadecan-6-yl)carbamate (2-8c)

Compound 2-8c (65 mg, 98% purity) was obtained according to the procedure described in Step 5 of Example 2-4.

MS (ESI) m/z: 1201.9 [M+H]⁺.

### Step 4

### (S)-2-amino-N5-(((2R,3S,4R,5S)-5-(2-amino-2-oxoethyl)-3,4-dihydroxytetrahydrofuran-2-yl)methyl)-N1-((S)-1-(((S)-1-(((3-((S)-9-ethyl-5-fluoro-9-hydroxy-4-methyl-10,13-dioxo-2,3,9,10,13,15-hexahydro-1H,12H-113-benzo[de]pyrano[3′,4′:6,7]indolizino[1,2-b]quinolin-1-yl)propoxy)methyl)amino)-1-oxopropan-2-yl)amino)-3-methyl-1-oxobutan-2-yl)pentanediamide (2-8d)

Compound 2-8d (68 mg, crude) was obtained according to the procedure described in Step 6 of Example 2-4.

MS (ESI) m/z: 979.8 [M+H]⁺.

### Step 5

### (S)—N5-(((2R,3S,4R,5S)-5-(2-amino-2-oxoethyl)-3,4-dihydroxytetrahydrofuran-2-yl)methyl)-2-(3-(2-(2,5-dioxo-2,5-dihydro-1H-pyrrol-1-yl)acetamido)propanamido)-N1-((S)-1-(((S)-1-(((3-((S)-9-ethyl-5-fluoro-9-hydroxy-4-methyl-10,13-dioxo-2,3,9,10,13,15-hexahydro-11H,1211-113-benzo[de]pyrano[3′,4′:6,7]indolizino[1,2-b]quinolin-1-yl)propoxy)methyl)amino)-1-oxopropan-2-yl)amino)-3-methyl-1-oxobutan-2-yl)pentanediamide (2-8)

Compound 2-8 (25 mg, 95% purity) was obtained according to the procedure described in Step 7 of Example 2-4.

MS (ESI) m/z: 1187.9 [M+H]⁺.

### Example 2-2

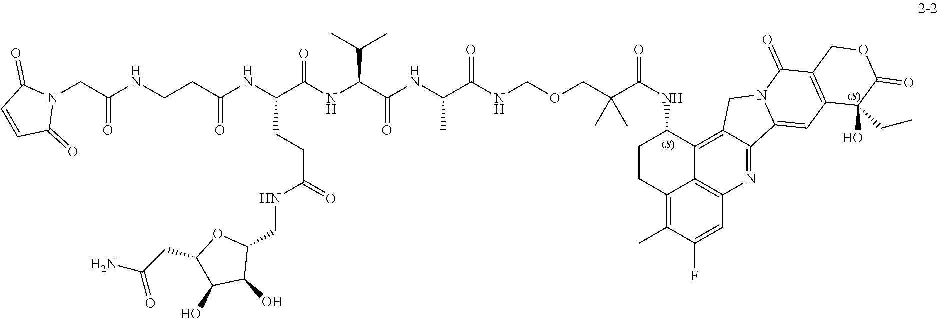

2-2 (38 mg, 96% purity) was synthesized according to general procedure B.

MS (ESI) m/z: 1266.7 [M+Na]-.

### Example 2-3

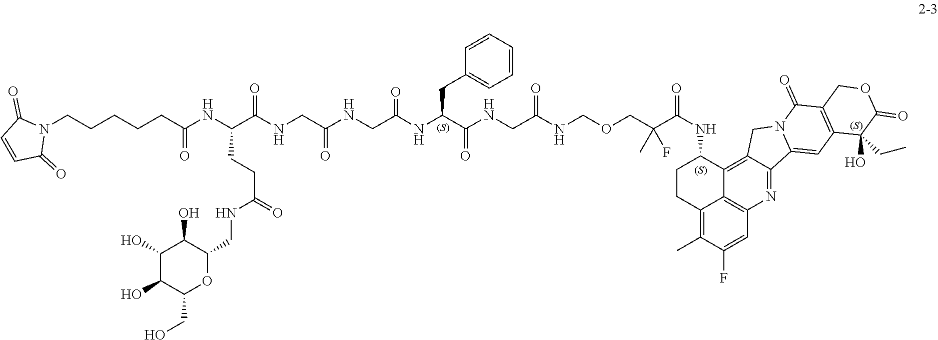

2-3 (30 mg, 98% purity) was synthesized according to general procedure A.

MS (ESI) m/z: 1408.1 [M+Na]^{T}.

### Example 2-5

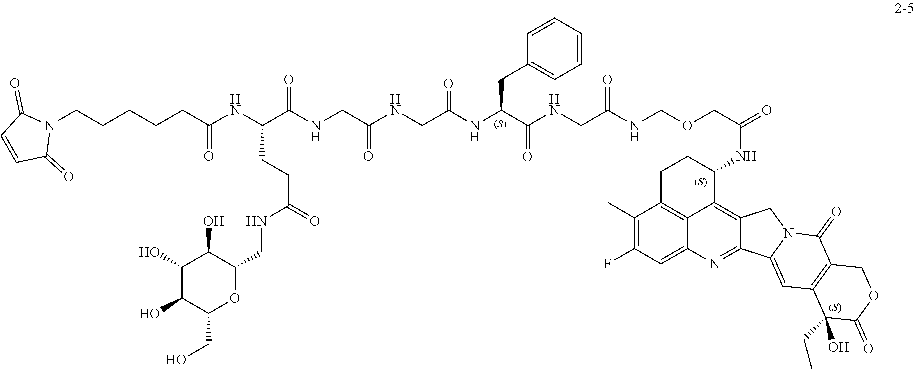

2-5 (51 mg, 97% purity) was synthesized according to general procedure C.

MS (ESI) m/z: 1292.0 [M+Na]-.

### Example 2-6

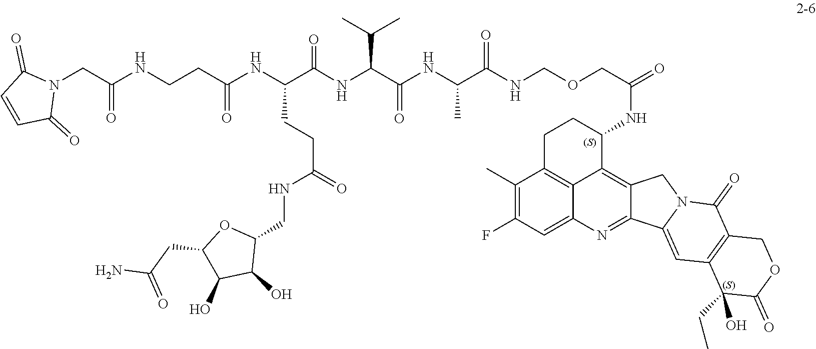

2-6 (34 mg, 95% purity) was synthesized according to general procedure B.

MS (ESI) m/z: 1255.0 [M+Na]

### Example 2-9

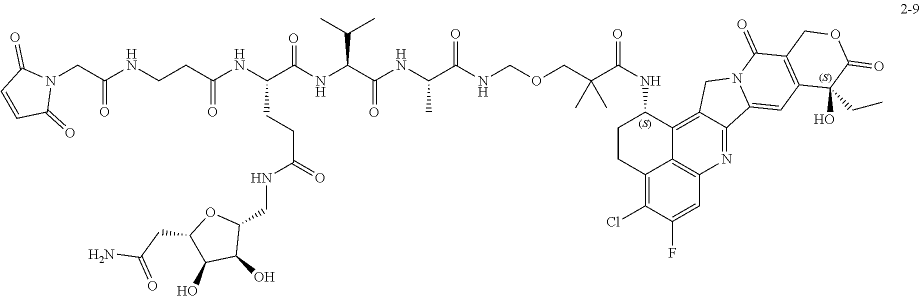

2-9 (9.7 mg, 99% purity) was synthesized according to general procedure B.

MS (ESI) m/z: 1287.0 [M+Na]^{T}.

### Example 2-10

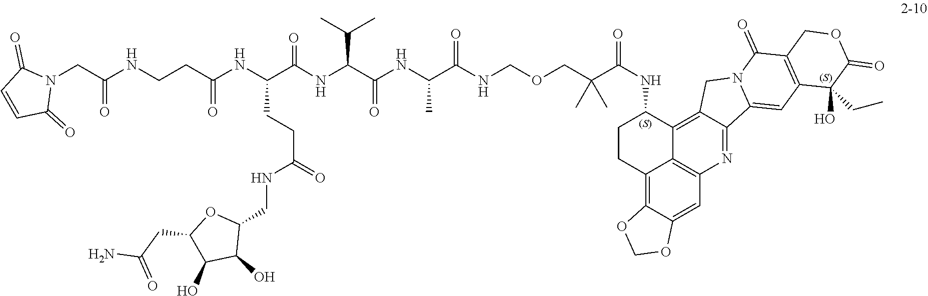

2-10 (15 mg, 95% purity) was synthesized according to general procedure B.

MS (ESI) m/z 1179.0 [M+Na]⁺.

### Example 2-11

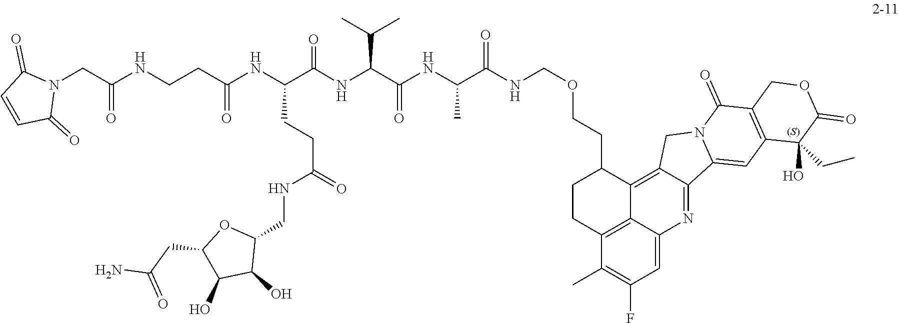

2-11 (23 mg, 98% purity) was synthesized according to general procedure D.

MS (ESI) m/z: 1173.9 [M+H]⁺.

### Example 2-12

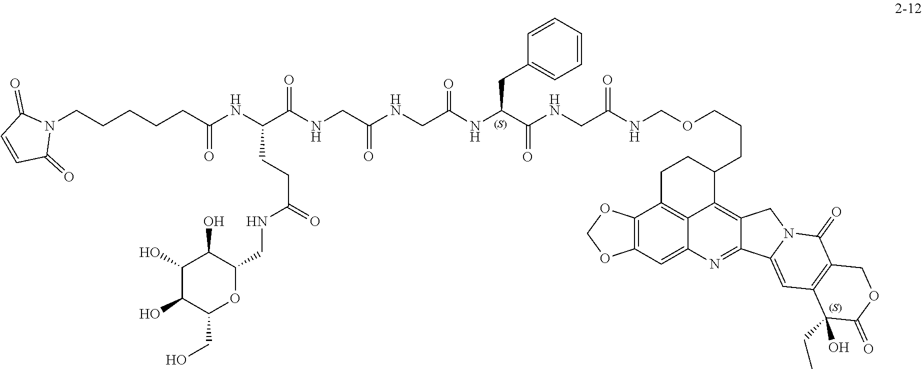

2-12 (4.1 mg, 95% purity) was synthesized according to general procedure C.

MS (ESI) m/z: 1337.0 [M+H]⁺.

### Example 2-13

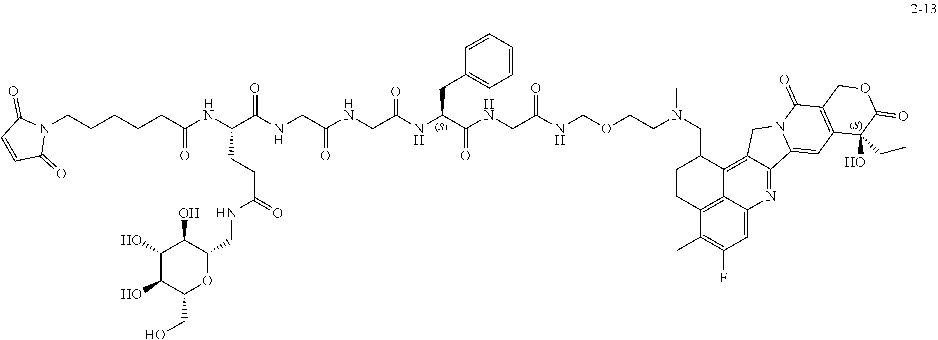

2-13 (13 mg, 98% purity) was synthesized according to general procedure C.

MS (ESI) m/z: 1375.2 [M+Na]+.

TABLE 1
Payload list

| Ex- | | |
|---|---|---|
| amples | Payload structure | Name |
| 1-A | 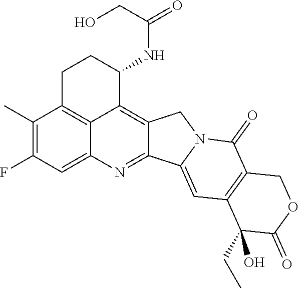 [SMILES: CC[C@@]1(O)C(=O)OCc2c1cc1n(c2=O)Cc2c-1nc1cc(F)c(C)c3c1c2[C@@H](NC(=O)CO)CC3] | DXd |
| 1-B | 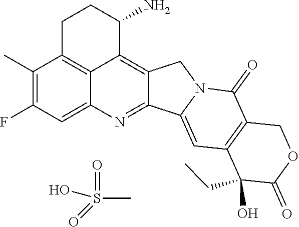 | Exatecan |
| 1-C | 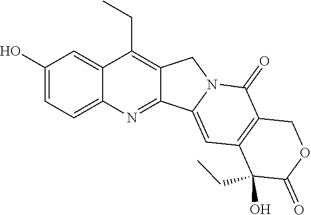 [SMILES: CCc1c2c(nc3ccc(O)cc13)-c1cc3c(c(=O)n1C2)COC(=O)[C@]3(O)CC] | SN38 |
| 1-1 | 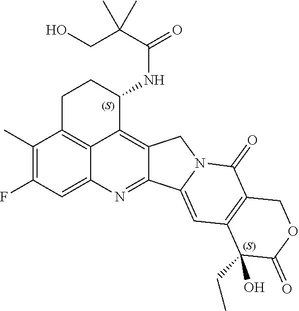 | 40410 |
| 1-2 | 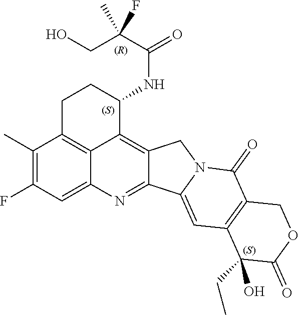 | 41127 |
| 1-3 | 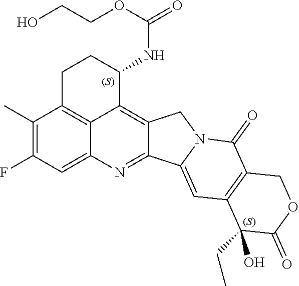 | 41129 |
| 1-4 | 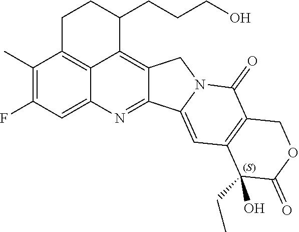 | 44009 |
| 1-5 | 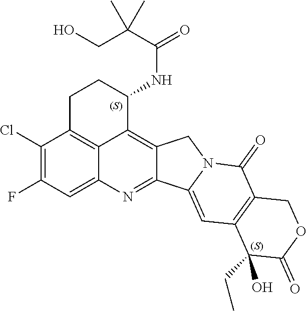 | 44411 |
| 1-6 | 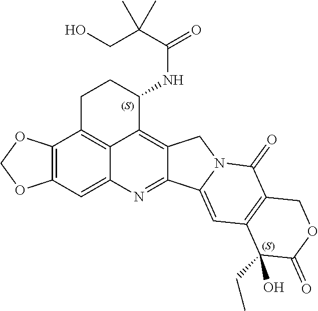 | 44475 |
| 1-7 | 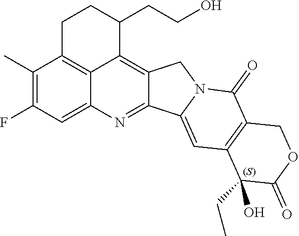 | 47397 |
| 1-8 | 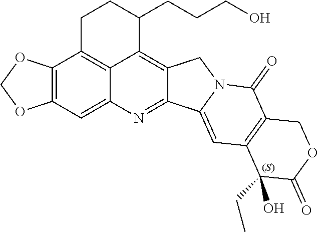 | 46879 |
| 1-9 | 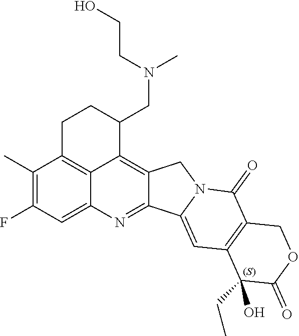 | 46035 |

TABLE 2
Linker-payload list

| Lin- | |
|---|---|
| ker | |
| Pay- | |
| load | |
| Num- | |
| ber | Linker-Payload Structure |
| 2-1 | 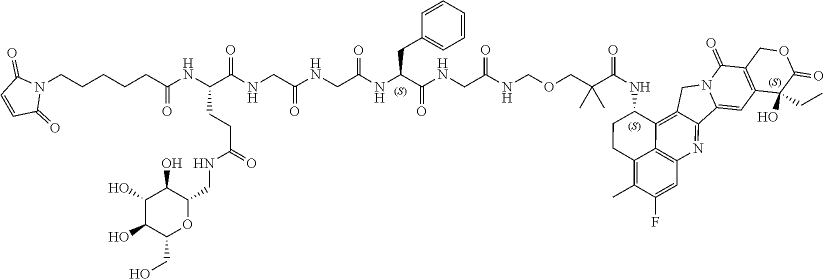 |
| 2-2 | 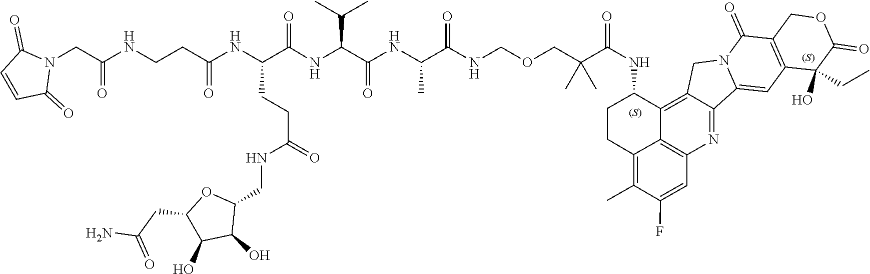 |
| 2-3 | 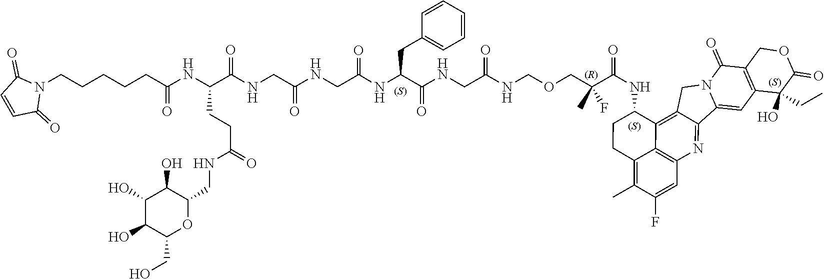 |
| 2-4 | 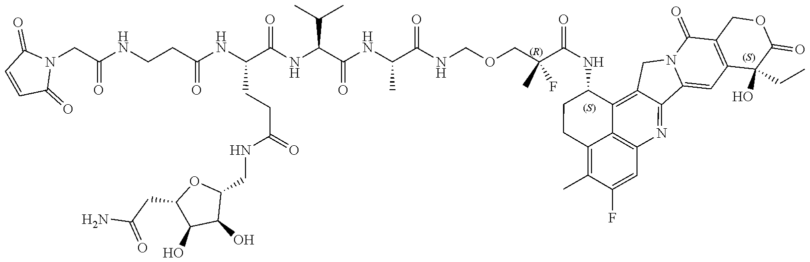 |
| 2-5 | 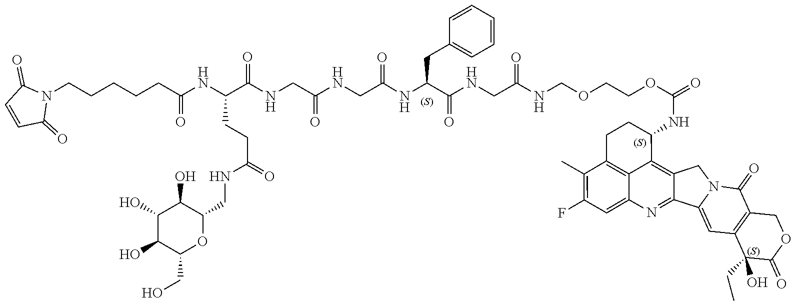 |
| 2-6 | 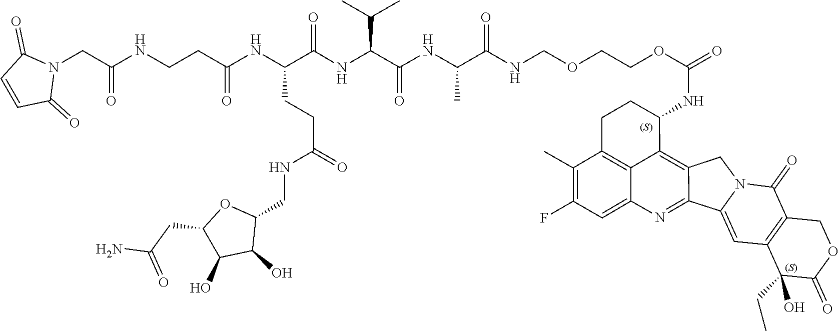 |
| 2-7 | 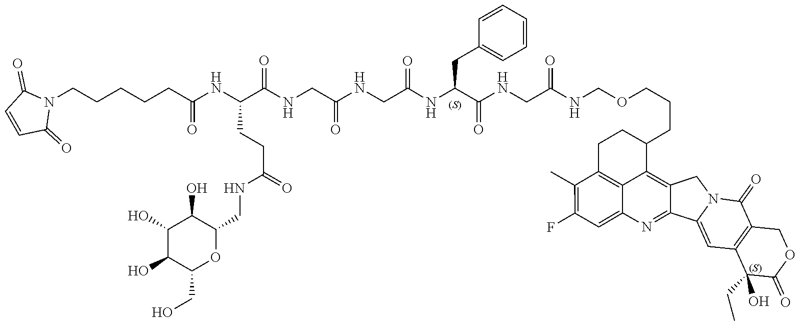 |
| 2-8 | 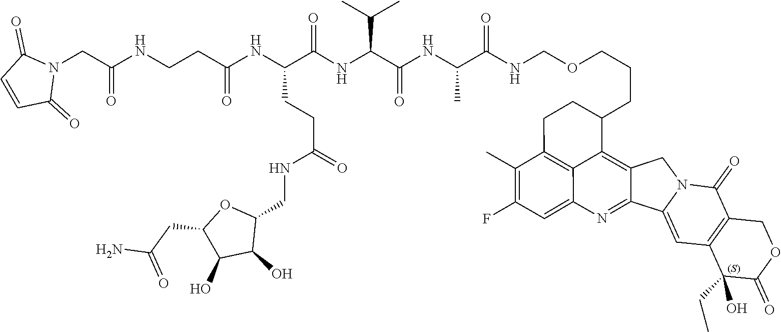 |
| 2-9 | 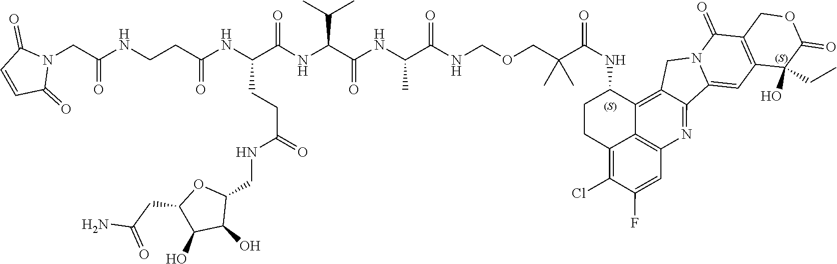 |
| 2-10 | 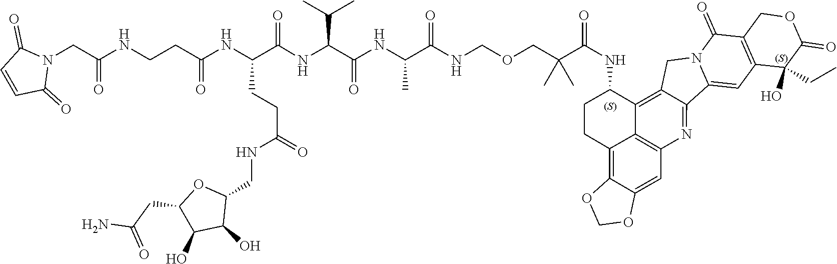 |
| 2-11 | 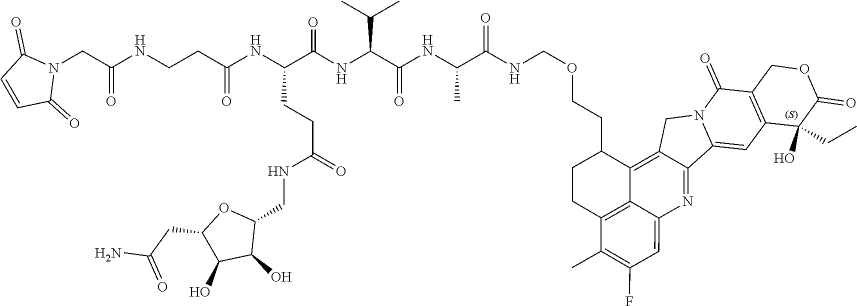 |
| 2-12 | 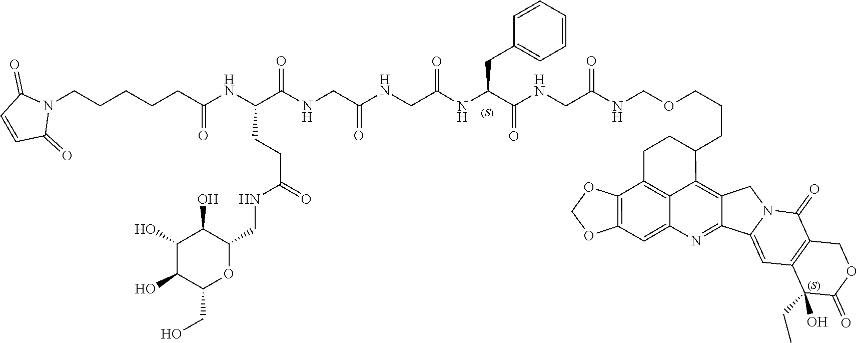 |
| 2-13 | 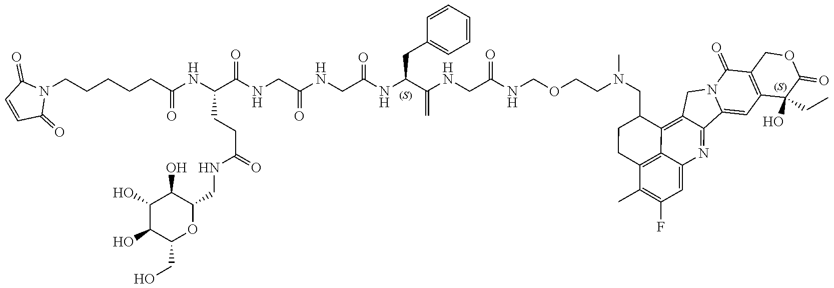 |

### Example 3

### ADC Preparation and Characterization

### Antibody Drug Conjugate Preparation

### Drug-to-Antibody Ratio (DAR) 8 Antibody Drug Conjugate Preparation

Antibody in conjugation buffer (with concentration 0.5-25 mg/mL, PBS buffer pH 6.0-8.5) was incubated under reduction temperature (0-40° C.) for 10 min and 8-15 eq. TCEP solution (5 mM stock in PBS buffer) was added into the reaction mixture and the reduction reaction was left for 1-8 hours at reduction temperature. Organic solvent (eg: DMSO, DMF, DMA, PG, acetonitrile, 0-25% v/v) and linker-payload stock (10-25 eq, 10 mM stock in organic solvent) were added stepwise after reduction mixture was cooled down to 0-25° C. Conjugation solution was left for 1-3 h at 0-25° C. and the reaction was quenched with N-acetyl cysteine (1 mM stock). The solution was submitted to buffer exchange (spin desalting column, ultrafiltration, and dialysis) into storage buffer (for example: pH 5.5-6.5 histidine acetate buffer, with optional additive such as sucrose, trehalose, tween 20, 60, 80).

### DAR4 Antibody Drug Conjugate Preparation

Antibody in conjugation buffer (with concentration 0.5-25 mg/mL, PBS buffer pH 6.0-7.4) was incubated under reduction temperature (0-37° C.) for 10 min and 1-6 eq. TCEP solution (5 mM stock in PBS buffer) was added into the reaction mixture and the reduction reaction was left or 1-18 hours at reduction temperature. Organic solvent (eg: DMSO, DMF, DMA, PG, acetonitrile, 0-25% v/v) and linker-payload stock (4-15 eq, 10 mM stock in organic solvent) were added stepwise after reduction. Conjugation solution was left for 1-3 h at 0-25° C. and the reaction was quenched with N-acetyl cysteine (1 mM stock). The solution was submitted to buffer exchange (spin desalting column, ultrafiltration, and dialysis) into storage buffer (for example: pH 5.5-6.5 histidine acetate buffer, with optional additive such as sucrose, trehalose, tween 20, 60, 80).

### DAR6 Antibody Drug Conjugate Preparation

Antibody in conjugation buffer (with concentration 0.5-25 mg/mL, PBS buffer pH 6.0-7.4) was incubated under reduction temperature (0-37° C.) for 10 min and 1-10 eq. TCEP solution (5 mM stock in PBS buffer) was added into the reaction mixture and the reduction reaction was left for 1-18 hours at reduction temperature. Organic solvent (eg: DMSO, DMF, DMA, PG, acetonitrile, 0-25% v/v) and linker-payload stock (6-25 eq, 10 mM stock in organic solvent) were added stepwise after reduction. Conjugation solution was left for 1-3 h at 0-25° C. and the reaction was quenched with N-acetyl cysteine (1 mM stock). The solution was submitted to buffer exchange (spin desalting column, ultrafiltration, and dialysis) into storage buffer (for example: pH 5.5-6.5 histidine acetate buffer, with optional additive such as sucrose, trehalose, tween 20, 60, 80).

### Example 4

### ADC3-A Antibody Drug Conjugate Preparation

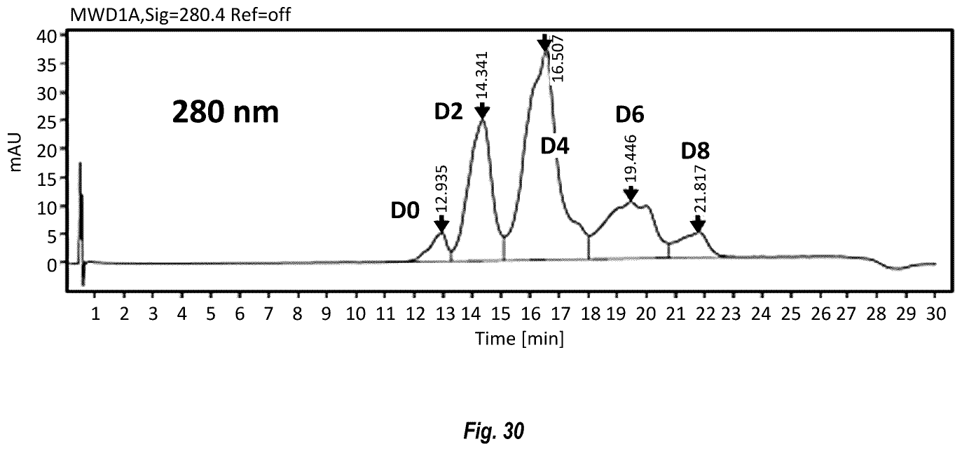

Antibody in conjugation buffer (with concentration 0.5-25 mg/mL, PBS buffer pH 6.0-7.0) was incubated under reduction temperature (0-5° C.) for 10 min and 1-4 eq. TCEP solution (5 mM stock in PBS buffer) was added into the reaction mixture and the reduction reaction was left for 1-18 hours at reduction temperature. Organic solvent (eg: DMSO, DMF, DMA, PG, acetonitrile, 0-25% v/v) and linker-payload stock (10-25 eq, 10 mM stock in organic solvent) were added stepwise after reduction. Conjugation solution was left for 1-3 h at 0-25° C. and the reaction was quenched with N-acetyl cysteine (1 mM stock). The solution was submitted to buffer exchange (spin desalting column, ultrafiltration, and dialysis) into storage buffer (for example: pH 5.5-6.5 histidine acetate buffer, with optional additive such as sucrose, trehalose, tween 20, 60, 80). The obtained ADC3-A, D4 species was in a range of 40-50% (FIG. 30).

ADC3-A is the biosimilar ADC of DS7300a (benchmark). The average DAR 3.99 was obtained and determined via HIC method 2 described below.

### Example 5

Maleimide hydrolysis process for ring open AIDCs (Examples ADC3-2, ADC3-4, ADC3-6, ADC3-8, ADC3-9, ADC3-10, ADC3-11, ADC3-16)

After the linker-payload conjugation step, the resulting ADCs underwent buffer exchange either through Amicon® ultrafiltration or desalting into basic buffer (pH 8.5-9.5, tris or borate acetate buffer). The reaction mixture was left for 18-24 h and maleimide process was monitored by LCMS. After the maleimide hydrolysis reached >90%, the resulting ADCs were subjected to buffer exchange into the formulation buffer (pH 5.5-6.5 histidine acetate buffer, with optional additive such as sucrose, trehalose, tween 20, 60, 80).

### Example 6

### ADC Characterization

Disclosed ADC examples were prepared by following the above procedures with DAR 8 profile. All ADCs were characterized via the following analytical methods.

Drug to antibody ratio (DAR) of the disclosed ADCs were determined by LCMS method or HIC (hydrophobicity interaction column) method.

SEC purity of disclosed ADCs are all >95% purity.

### LCMS Method for DAR Determination

LC-MS analysis was carried out under the following measurement conditions:
LC-MS system: Vanquish™ Flex UHPLC and Orbitrap Exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Injected sample amount: 1 Ig
MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

### HIC Method for DAR Determination

HPLC analysis was carried out under the following measurement conditions:
HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM phosphate buffer, pH 7.0
Mobile phase B: 50 mM phosphate buffer, 25% (V/V) isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg
SEC method to determine ADC purity
HPLC analysis was carried out under the following measurement conditions:
HPLC system: Waters H-Class UPLC System
Detector: measurement wavelength: 280 nm
Column: ACQUITY UPLC BEH200 SEC 1.7 μm 4.6×150 mm, Waters
Column temperature: room temperature
Mobile phase A: 200 mM phosphate buffer, 250 mM potassium chloride, 15% isopropyl alcohol, pH 7.0
Gradient program: under 10 min isocratic elutions with the flow rate of 0.3 mL/min
Injected sample amount: 20 μg
HIC methods to evaluate ADC hydrophobicity
HIC Method 1
HPLC analysis was carried out under the following measurement conditions:
HPLC system: Waters ACQUITY ARC HPLC System
Detector: measurement wavelength: 280 nm
Column: Tosoh Bioscience 4.6 μm ID×3.5 cm, 2.5 km butyl-nonporous resin column
Column temperature: 25° C.
Mobile phase A: 1.5 M ammonium sulfate, 50 mM phosphate buffer, pH 7.0
Mobile phase B: 50 mM phosphate buffer, 25% (V/V) isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 g

### HIC Method 2

HPLC analysis was carried out under the following measurement conditions:
HPLC system: Waters ACQUITY ARC HPLC System
Detector: measurement wavelength: 280 nm
Column: MABPac HIC-10, 5 μm, 4.6×10 mm (Thermo)
Column temperature: 25° C.
Mobile phase A: 1.5 M ammonium sulfate, 50 mM sodium phosphate, pH 7.0
Mobile phase B: 50 mM sodium phosphate, pH 7.0
Gradient program: 20% B—20% B (0 min-1 min), 0% B-0% B (1 min-35 min), 20% B-20% B (35 min-40 min)
Flow rate: 0.5 mL/min

Sample preparation: The sample was diluted with initial mobile phase to 0.5 mg/mL.

DAR8 HIC retention time (min) is an indication of the relative hydrophobicity of an ADC. ADCs with higher DAR8 HIC retention have a higher hydrophobic property. As shown in Table 3 below, the disclosed ADCs are more hydrophilic than reference ADCs ADC3-A and ADC3-B.

TABLE 3
ADCs

| | | | HIC |
|---|---|---|---|
| | | | DAR8 |
| | | | RT |
| ADC | | | (min) |
| ex- | Anti- | | Meth- |
| amples | body | ADC structures | od 1 |
| ADC3-A | MABX- 9001a | 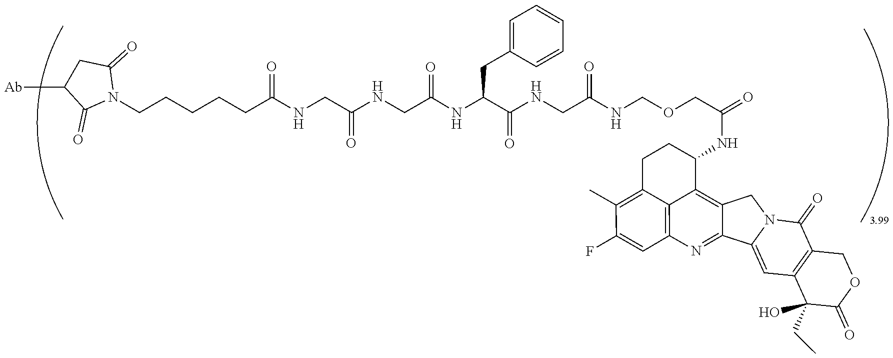 | 21.817 (Meth - od 2) |
| ADC3-B | MABX- 9001a | 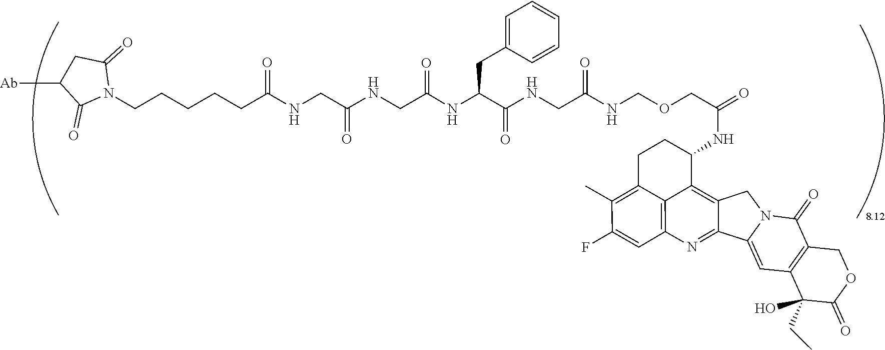 | 10.86 |
| ADC3-1 | BGA- 6938 | 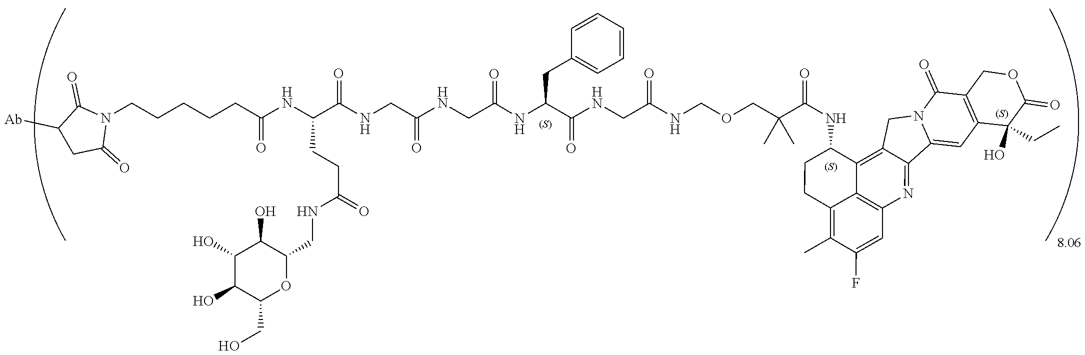 | 10.315 |
| ADC3-2 | BGA- 6938 | 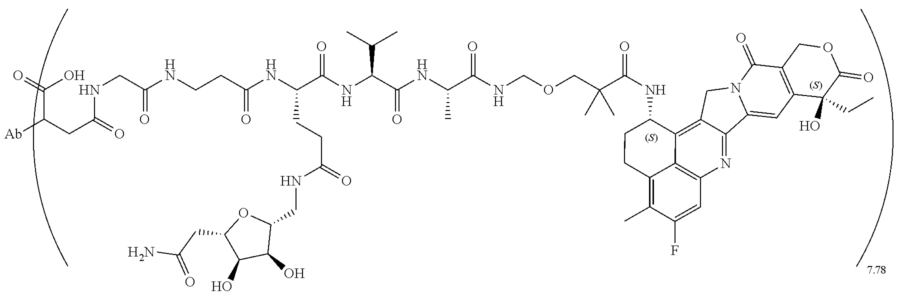 | 10.098 |
| ADC3-3 | BGA- 6938 | 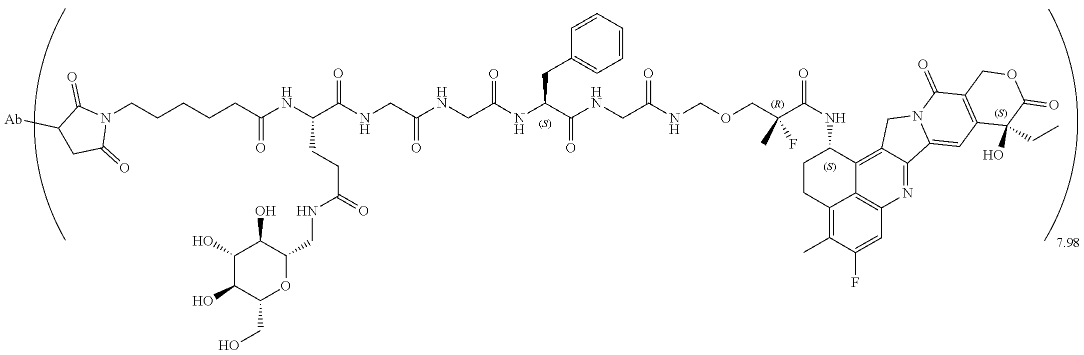 | 9.85 |
| ADC3-4 | BGA- 6938 | 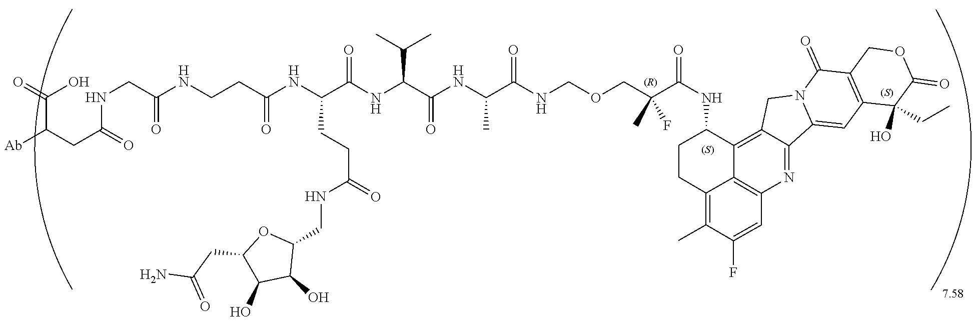 | 9.562 |
| ADC3-5 | BGA- 6938 | 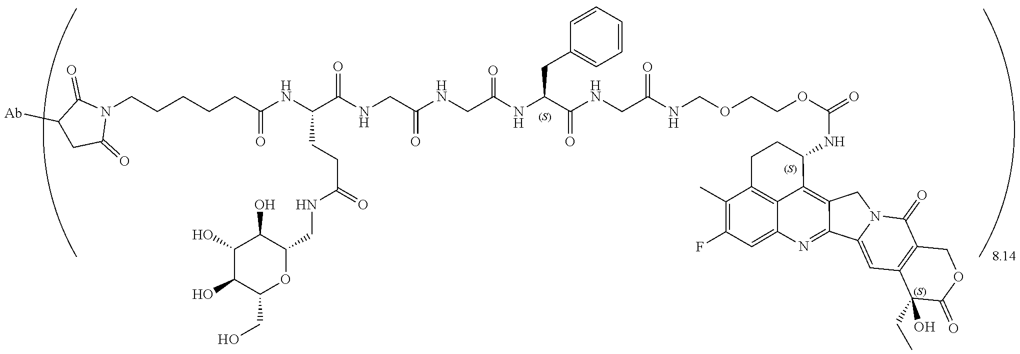 | 9.711 |
| ADC 3-6 | BGA- 6938 | 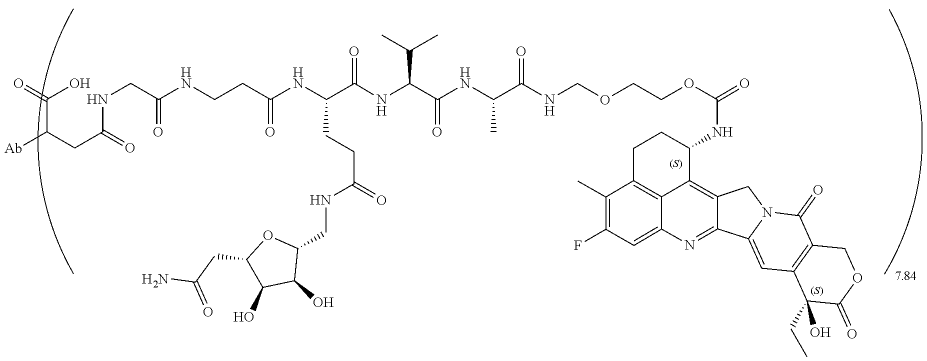 | 9.627 |
| ADC3-7 | BGA- 6938 | 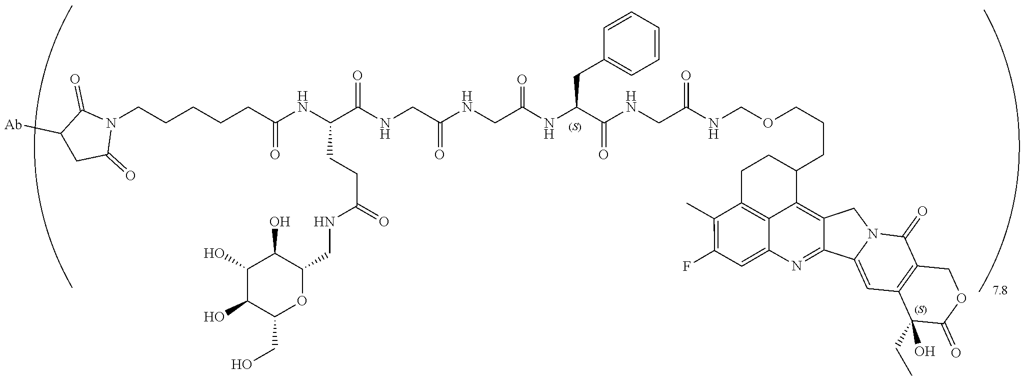 | 10.021 |
| ADC3-8 | BGA- 6938 | 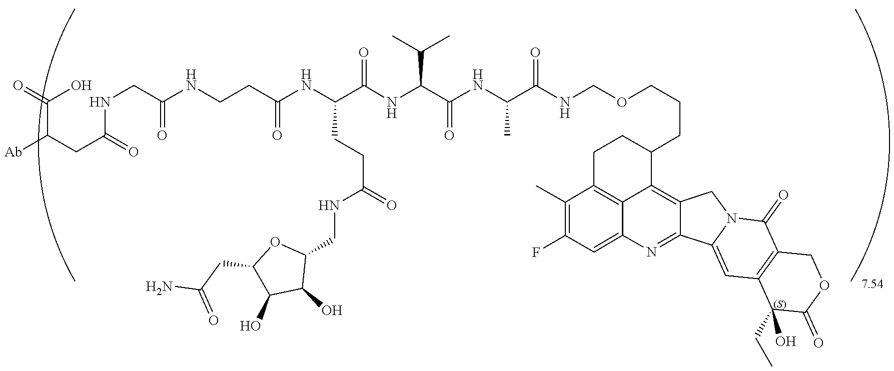 | 10.196 |
| ADC3-9 | BGA- 6938 | 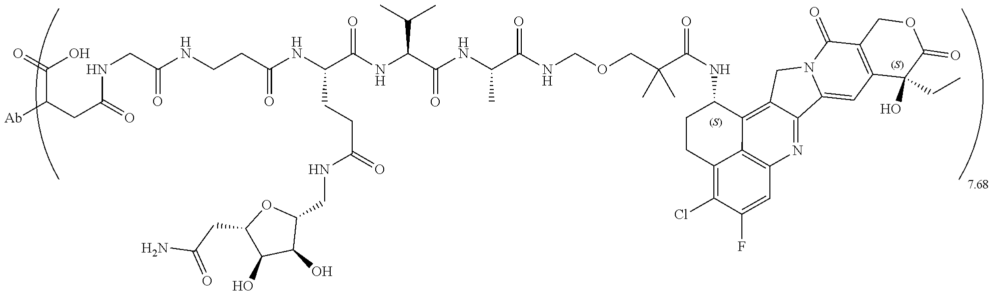 | 10.232 |
| ADC3- 10 | BGA- 6938 | 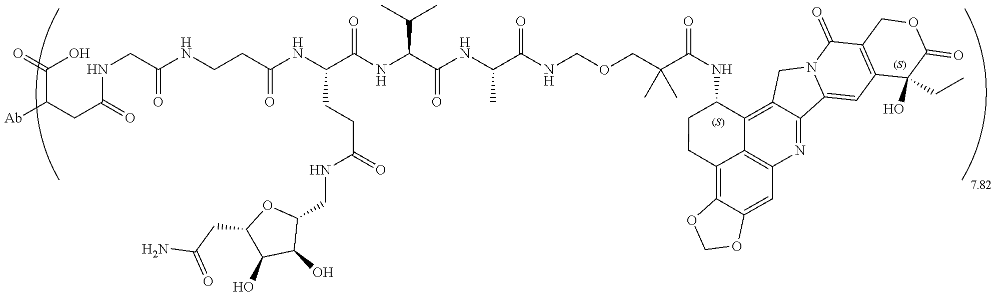 | 9.853 |
| ADC3- 11 | BGA- 6938 | 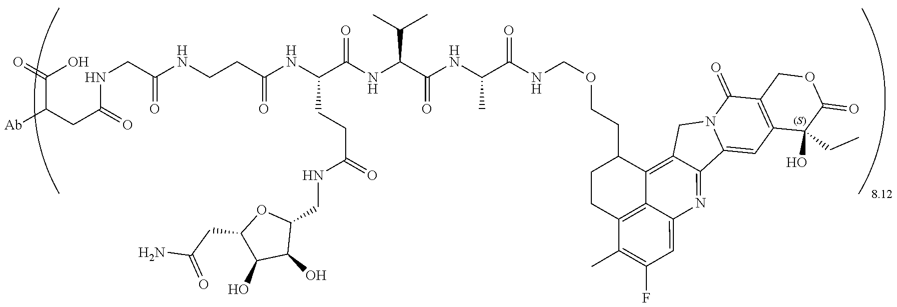 | 9.925 |
| ADC3- 12 | BGA- 6938 | 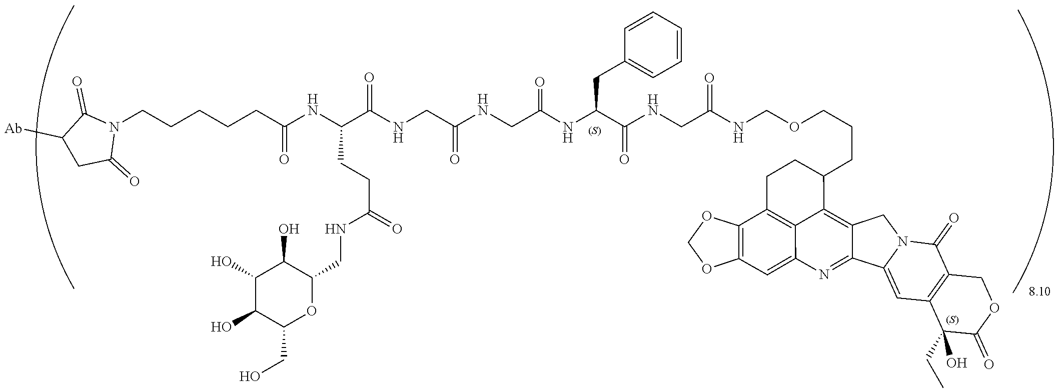 | 9.724 |
| ADC3- 13 | BGA- 6938 | 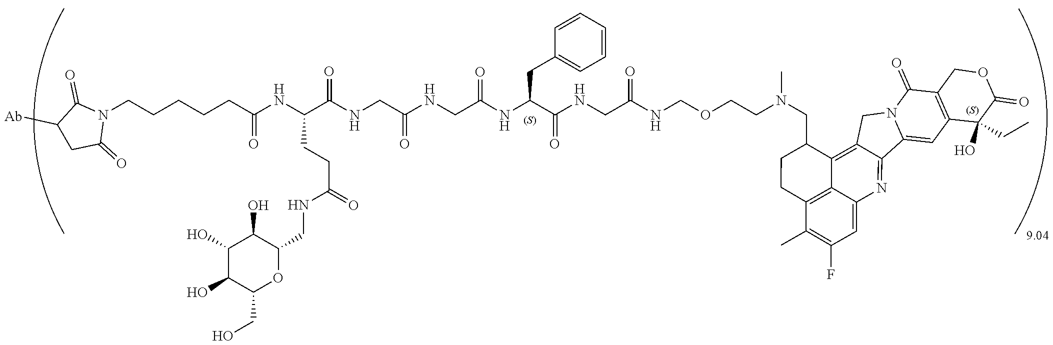 | 9.375 |
| ADC3- 14 | BGA- 5063 | 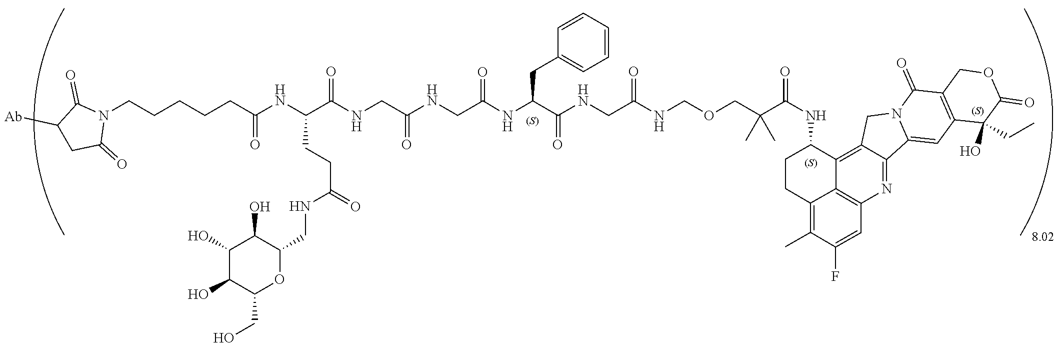 | 10.303 |
| ADC3- 15 | BGA- 5063 | 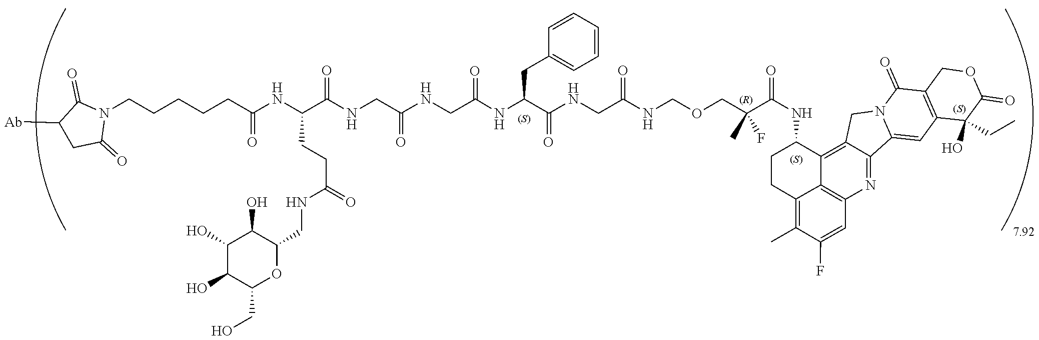 | 9.741 |
| ADC3- 16 | BGA- 5063 | 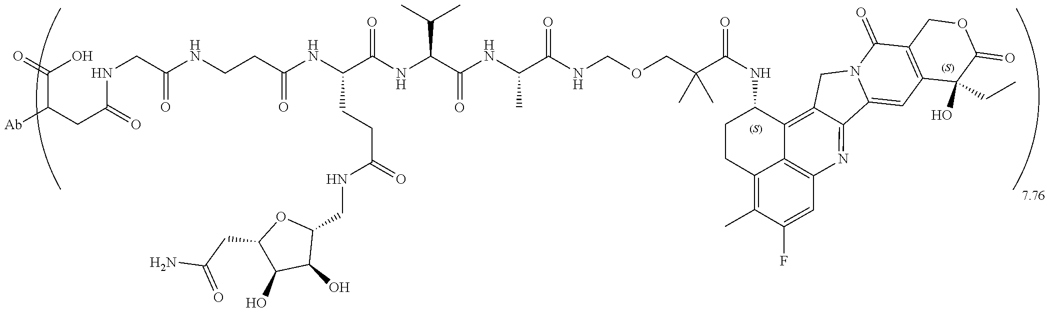 | 10.093 |

### Example 7. Antibody Information

The antibodies and antigen binding fragments thereof provided herein can be prepared by methods known in the art. The sequences of the exemplary antibodies and antigen binding fragments are provided in the below table.

TABLE 4
Sequences of antibodies

| | SEQ ID | | |
|---|---|---|---|
| Antibody | NO | Description | SEQUENCE |
| MABX- | 80 | Light Chain | EIVLTQSPATLSLSPGERATLSCRASSRLIYMHWYQQKPGQAPRPLIYATSNLASGIPARESGS |
| 900la | | Sequence | GSGTDFTLTISSLEPEDFAVYYCQQWNSNPPTFGQGTKVEIKRTVAAPSVFIFPPSDEQLKSGT |
| (DS7300a | | | ASVVCLLNNFYPREAKVQWKVDNALQSGNSQESVTEQDSKDSTYSLSSTLTLSKADYEKHKVYA |
| antibody) | | | CEVTHQGLSSPVTKSENRGEC |
| | 81 | Heavy Chain | QVQLVQSGAEVKKPGSSVKVSCKASGYTFTNYVMHWVRQAPGQGLEWMGYINPYNDDVKYNEKE |
| | | Sequence | KGRVTITADESTSTAYMELSSLRSEDTAVYYCARWGYYGSPLYYFDYWGQGTLVTVSSASTKGP |
| | | | SVFPLAPSSKSTSGGTAALGCLVKDYFPEPVTVSWNSGALTSGVHTFPAVLQSSGLYSLSSVVT |
| | | | VPSSSLGTQTYICNVNHKPSNTKVDKRVEPKSCDKTHTCPPCPAPELLGGPSVFLFPPKPKDTL |
| | | | MISRTPEVTCVVVDVSHEDPEVKFNWYVDGVEVHNAKTKPREEQYNSTYRVVSVLTVLHQDWIN |
| | | | GKEYKCKVSNKALPAPIEKTISKAKGQPREPQVYTLPPSREEMTKNQVSLTCLVKGFYPSDIAV |
| | | | EWESNGQPENNYKTTPPVLDSDGSFFLYSKLTVDKSRWQQGNVFSCSVMHEALHNHYTQKSLSL |
| | | | SPGK |
| BGA-3726 | 1 | HCDR1 (Kabat) | TSNWWN |
| | 2 | HCDR2 (Kabat) | EIYPTGNTNYSPSLKS |
| | 3 | HCDR3 (Kabat) | GWNWEDP |
| | 4 | LCDR1 (Kabat) | RTSQSINGILLA |
| | 5 | LCDR2 (Kabat) | GASSRAT |
| | 6 | LCDR3 (Kabat) | QQYGSSPRT |
| | 7 | VH AA | QVQLQESGPGLVKPSGTLSLTCAVSGGSISTSNWWNWVRQSPGKGLEWIGETYPTGNTNYSPSL |
| | | | KSRVTISIDKSKNQFSLKLNSVTAADTAVYYCARGWNWEDPWGQGTLVTVSS |
| | 8 | VL AA | EIVLTQSPGTLSLSPGERATLSCRTSQSINGILLAWYQQKPGQAPRLLIYGASSRATGIPDRES |
| | | | GSGSGTDFTLSISRLEPEDFAVYYCQQYGSSPRTFGQGTKVEIK |
| | 9 | VH DNA | CAGGTGCAGCTGCAGGAGTCGGGCCCAGGACTGGTGAAGCCTTCGGGGACCCTGTCCCTCACCT |
| | | | GCGCTGTCTCTGGTGGCTCCATCAGTACTAGTAACTGGTGGAATTGGGTCCGCCAGTCCCCAGG |
| | | | GAAGGGGCTGGAGTGGATTGGAGAAATCTATCCTACTGGGAACACCAACTACAGCCCGTCCCTC |
| | | | AAGAGTCGAGTCACCATATCAATAGACAAGTCCAAGAACCAGTTCTCCCTGAAACTTAATTCTG |
| | | | TGACCGCCGCGGACACGGCCGTGTATTATTGTGCCAGAGGATGGAACTGGTTCGACCCCTGGGG |
| | | | CCAGGGAACCCTGGTCACCGTCTCCTCA |
| | 10 | VL DNA | GAAATTGTGTTGACGCAGTCTCCAGGCACCCTGTCTTTGTCTCCAGGGGAAAGAGCCACCCTCT |
| | | | CCTGCAGGACCAGTCAGAGTATTAACGGCATCTTGTTAGCCTGGTACCAGCAGAAACCTGGCCA |
| | | | GGCTCCCAGGCTCCTCATCTATGGTGCATCCAGCAGGGCCACTGGCATCCCCGACAGGTTCAGT |
| | | | GGCAGTGGGTCTGGGACAGACTTCACTCTCAGCATCAGCAGACTGGAGCCTGAAGATTTTGCAG |
| | | | TGTATTACTGTCAGCAGTATGGCAGTTCACCTCGGACGTTCGGCCAAGGGACCAAGGTGGAAAT |
| | | | CAAA |
| BGA-4826 | 11 | HCDR1 (Kabat) | TSNYWN |
| | 2 | HCDR2 (Kabat) | EIYPTGNTNYSPSLKS |
| | 3 | HCDR3 (Kabat) | GWNWFDP |
| | 4 | LCDR1 (Kabat) | RTSQSINGILLA |
| | 5 | LCDR2 (Kabat) | GASSRAT |
| | 6 | LCDR3 (Kabat) | QQYGSSPRT |
| | 12 | VH AA | QVQLQESGPGLVKPSGTLSLTCAVSGGSISTSNYWNWVRQSPGKGLEWIGEIYPTGNTNYSPSL |
| | | | KSRVTISIDKSKNQFSLKLNSVTAADTAVYYCARGWNWEDPWGQGTLVTVSS |
| | 00 | VL AA | EIVLTQSPGTLSLSPGERATLSCRTSQSINGILLAWYQQKPGQAPRLLIYGASSRATGIPDRES |
| | | | GSGSGTDFTLSISRLEPEDFAVYYCQQYGSSPRTFGQGTKVEIK |
| | 13 | VH DNA | CAGGTGCAGCTGCAGGAGTCGGGCCCAGGACTGGTGAAGCCTTCGGGGACCCTGTCCCTCACCT |
| | | | GCGCTGTCTCTGGTGGCTCCATCAGTACTAGTAACTACTGGAATTGGGTCCGCCAGTCCCCAGG |
| | | | GAAGGGGCTGGAGTGGATTGGAGAAATCTATCCTACTGGGAACACCAACTACAGCCCGTCCCTC |
| | | | AAGAGTCGAGTCACCATATCAATAGACAAGTCCAAGAACCAGTTCTCCCTGAAACTTAATTCTG |
| | | | TGACCGCCGCGGACACGGCCGTGTATTATTGTGCCAGAGGATGGAACTGGTTCGACCCCTGGGG |
| | | | CCAGGGAACCCTGGTCACCGTCTCCTCA |
| | 10 | VL DNA | GAAATTGTGTTGACGCAGTCTCCAGGCACCCTGTCTTTGTCTCCAGGGGAAAGAGCCACCCTCT |
| | | | CCTGCAGGACCAGTCAGAGTATTAACGGCATCTTGTTAGCCTGGTACCAGCAGAAACCTGGCCA |
| | | | GGCTCCCAGGCTCCTCATCTATGGTGCATCCAGCAGGGCCACTGGCATCCCCGACAGGTTCAGT |
| | | | GGCAGTGGGTCTGGGACAGACTTCACTCTCAGCATCAGCAGACTGGAGCCTGAAGATTTTGCAG |
| | | | TGTATTACTGTCAGCAGTATGGCAGTTCACCTCGGACGTTCGGCCAAGGGACCAAGGTGGAAAT |
| | | | CAAA |
| BGA-4498 | 1 | HCDR1 (Kabat) | TSNWWN |
| | 2 | HCDR2 (Kabat) | EIYPTGNTNYSPSLKS |
| | 14 | HCDR3 (Kabat) | GYNWFDP |
| | 4 | LCDR1 (Kabat) | RTSQSINGILLA |
| | 5 | LCDR2 (Kabat) | GASSRAT |
| | 6 | LCDR3 (Kabat) | QQYGSSPRT |
| | 15 | VH AA | QVQLQESGPGLVKPSGTLSLTCAVSGGSISTSNWWNWVRQSPGKGLEWIGEIYPTGNTNYSPSL |
| | | | KSRVTISIDKSKNQFSLKLNSVTAADTAVYYCARGYNWFDPWGQGTLVTVSS |
| | 8 | VL AA | EIVLTQSPGTLSLSPGERATLSCRTSQSINGILLAWYQQKPGQAPRLLIYGASSRATGIPDRES |
| | | | GSGSGTDFTLSISRLEPEDFAVYYCQQYGSSPRTFGQGTKVEIK |
| | 16 | VH DNA | CAGGTGCAGCTGCAGGAGTCGGGCCCAGGACTGGTGAAGCCTTCGGGGACCCTGTCCCTCACCT |
| | | | GCGCTGTCTCTGGTGGCTCCATCAGTACTAGTAACTGGTGGAATTGGGTCCGCCAGTCCCCAGG |
| | | | GAAGGGGCTGGAGTGGATTGGAGAAATCTATCCTACTGGGAACACCAACTACAGCCCGTCCCTC |
| | | | AAGAGTCGAGTCACCATATCAATAGACAAGTCCAAGAACCAGTTCTCCCTGAAACTTAATTCTG |
| | | | TGACCGCCGCGGACACGGCCGTGTATTATTGTGCCAGAGGATACAACTGGTTCGACCCCTGGGG |
| | | | CCAGGGAACCCTGGTCACCGTCTCCTCA |
| | 10 | VL DNA | GAAATTGTGTTGACGCAGTCTCCAGGCACCCTGTCTTTGTCTCCAGGGGAAAGAGCCACCCTCT |
| | | | CCTGCAGGACCAGTCAGAGTATTAACGGCATCTTGTTAGCCTGGTACCAGCAGAAACCTGGCCA |
| | | | GGCTCCCAGGCTCCTCATCTATGGTGCATCCAGCAGGGCCACTGGCATCCCCGACAGGTTCAGT |
| | | | GGCAGTGGGTCTGGGACAGACTTCACTCTCAGCATCAGCAGACTGGAGCCTGAAGATTTTGCAG |
| | | | TGTATTACTGTCAGCAGTATGGCAGTTCACCTCGGACGTTCGGCCAAGGGACCAAGGTGGAAAT |
| | | | CAAA |
| BGA-4269 | 1 | HCDR1 (Kabat) | TSNWWN |
| | 2 | HCDR2 (Kabat) | EIYPTGNTNYSPSLKS |
| | 17 | HCDR3 (Kabat) | GWNYFDP |
| | 4 | LCDR1 (Kabat) | RTSQSINGILLA |
| | 5 | LCDR2 (Kabat) | GASSRAT |
| | 6 | LCDR3 (Kabat) | QQYGSSPRT |
| | 18 | VH AA | QVQLQESGPGLVKPSGTLSLTCAVSGGSISTSNWWNWVRQSPGKGLEWIGEIYPTGNTNYSPSL |
| | | | KSRVTISIDKSKNQFSLKLNSVTAADTAVYYCARGWNYFDPWGQGTLVTVSS |
| | 8 | VL AA | EIVLTQSPGTLSLSPGERATLSCRTSQSINGILLAWYQQKPGQAPRLLIYGASSRATGIPDRES |
| | | | GSGSGTDFTLSISRLEPEDFAVYYCQQYGSSPRTFGQGTKVEIK |
| | 19 | VH DNA | CAGGTGCAGCTGCAGGAGTCGGGCCCAGGACTGGTGAAGCCTTCGGGGACCCTGTCCCTCACCT |
| | | | GCGCTGTCTCTGGTGGCTCCATCAGTACTAGTAACTGGTGGAATTGGGTCCGCCAGTCCCCAGG |
| | | | GAAGGGGCTGGAGTGGATTGGAGAAATCTATCCTACTGGGAACACCAACTACAGCCCGTCCCTC |
| | | | AAGAGTCGAGTCACCATATCAATAGACAAGTCCAAGAACCAGTTCTCCCTGAAACTTAATTCTG |
| | | | TGACCGCCGCGGACACGGCCGTGTATTATTGTGCCAGAGGATGGAACTACTTCGACCCCTGGGG |
| | | | CCAGGGAACCCTGGTCACCGTCTCCTCA |
| | 10 | VL DNA | GAAATTGTGTTGACGCAGTCTCCAGGCACCCTGTCTTTGTCTCCAGGGGAAAGAGCCACCCTCT |
| | | | CCTGCAGGACCAGTCAGAGTATTAACGGCATCTTGTTAGCCTGGTACCAGCAGAAACCTGGCCA |
| | | | GGCTCCCAGGCTCCTCATCTATGGTGCATCCAGCAGGGCCACTGGCATCCCCGACAGGTTCAGT |
| | | | GGCAGTGGGTCTGGGACAGACTTCACTCTCAGCATCAGCAGACTGGAGCCTGAAGATTTTGCAG |
| | | | TGTATTACTGTCAGCAGTATGGCAGTTCACCTCGGACGTTCGGCCAAGGGACCAAGGTGGAAAT |
| | | | CAAA |
| BGA-4380 | 1 | HCDR1 (Kabat) | TSNWWN |
| | 2 | HCDR2 (Kabat) | EIYPTGNTNYSPSLKS |
| | 3 | HCDR3 (Kabat) | GWNWEDP |
| | 20 | LCDR1 (Kabat) | RTSQSIQGILLA |
| | 5 | LCDR2 (Kabat) | GASSRAT |
| | 6 | LCDR3 (Kabat) | QQYGSSPRT |
| | 7 | VH AA | QVQLQESGPGLVKPSGTLSLTCAVSGGSISTSNWWNWVRQSPGKGLEWIGEIYPTGNTNYSPSL |
| | | | KSRVTISIDKSKNQFSLKLNSVTAADTAVYYCARGWNWEDPWGQGTLVTVSS |
| | 21 | VL AA | EIVLTQSPGTLSLSPGERATLSCRTSQSIQGILLAWYQQKPGQAPRLLIYGASSRATGIPDRES |
| | | | GSGSGTDFTLSISRLEPEDFAVYYCQQYGSSPRTFGQGTKVEIK |
| | 9 | VH DNA | CAGGTGCAGCTGCAGGAGTCGGGCCCAGGACTGGTGAAGCCTTCGGGGACCCTGTCCCTCACCT |
| | | | GCGCTGTCTCTGGTGGCTCCATCAGTACTAGTAACTGGTGGAATTGGGTCCGCCAGTCCCCAGG |
| | | | GAAGGGGCTGGAGTGGATTGGAGAAATCTATCCTACTGGGAACACCAACTACAGCCCGTCCCTC |
| | | | AAGAGTCGAGTCACCATATCAATAGACAAGTCCAAGAACCAGTTCTCCCTGAAACTTAATTCTG |
| | | | TGACCGCCGCGGACACGGCCGTGTATTATTGTGCCAGAGGATGGAACTGGTTCGACCCCTGGGG |
| | | | CCAGGGAACCCTGGTCACCGTCTCCTCA |
| | 22 | VL DNA | GAAATTGTGTTGACGCAGTCTCCAGGCACCCTGTCTTTGTCTCCAGGGGAAAGAGCCACCCTCT |
| | | | CCTGCAGGACCAGTCAGAGTATTCAGGGCATCTTGTTAGCCTGGTACCAGCAGAAACCTGGCCA |
| | | | GGCTCCCAGGCTCCTCATCTATGGTGCATCCAGCAGGGCCACTGGCATCCCCGACAGGTTCAGT |
| | | | GGCAGTGGGTCTGGGACAGACTTCACTCTCAGCATCAGCAGACTGGAGCCTGAAGATTTTGCAG |
| | | | TGTATTACTGTCAGCAGTATGGCAGTTCACCTCGGACGTTCGGCCAAGGGACCAAGGTGGAAAT |
| | | | CAAA |
| BGA-4265 | 1 | HCDR1 (Kabat) | TSNWWN |
| | 2 | HCDR2 (Kabat) | EIYPTGNTNYSPSLKS |
| | 3 | HCDR3 (Kabat) | GWNWFDP |
| | 23 | LCDR1 (Kabat) | RTSQSINAILLA |
| | 5 | LCDR2 (Kabat) | GASSRAT |
| | 6 | LCDR3 (Kabat) | QQYGSSPRT |
| | 7 | VH AA | QVQLQESGPGLVKPSGTLSLTCAVSGGSISTSNWWNWVRQSPGKGLEWIGEIYPTGNTNYSPSL |
| | | | KSRVTISIDKSKNQFSLKLNSVTAADTAVYYCARGWNWEDPWGQGTLVTVSS |
| | 24 | VL AA | EIVLTQSPGTLSLSPGERATLSCRTSQSINAILLAWYQQKPGQAPRLLIYGASSRATGIPDRES |
| | | | GSGSGTDFTLSISRLEPEDFAVYYCQQYGSSPRTFGQGTKVEIK |
| | 9 | VH DNA | CAGGTGCAGCTGCAGGAGTCGGGCCCAGGACTGGTGAAGCCTTCGGGGACCCTGTCCCTCACCT |
| | | | GCGCTGTCTCTGGTGGCTCCATCAGTACTAGTAACTGGTGGAATTGGGTCCGCCAGTCCCCAGG |
| | | | GAAGGGGCTGGAGTGGATTGGAGAAATCTATCCTACTGGGAACACCAACTACAGCCCGTCCCTC |
| | | | AAGAGTCGAGTCACCATATCAATAGACAAGTCCAAGAACCAGTTCTCCCTGAAACTTAATTCTG |
| | | | TGACCGCCGCGGACACGGCCGTGTATTATTGTGCCAGAGGATGGAACTGGTTCGACCCCTGGGG |
| | | | CCAGGGAACCCTGGTCACCGTCTCCTCA |
| | 25 | VL DNA | GAAATTGTGTTGACGCAGTCTCCAGGCACCCTGTCTTTGTCTCCAGGGGAAAGAGCCACCCTCT |
| | | | CCTGCAGGACCAGTCAGAGTATTAACGCCATCTTGTTAGCCTGGTACCAGCAGAAACCTGGCCA |
| | | | GGCTCCCAGGCTCCTCATCTATGGTGCATCCAGCAGGGCCACTGGCATCCCCGACAGGTTCAGT |
| | | | GGCAGTGGGTCTGGGACAGACTTCACTCTCAGCATCAGCAGACTGGAGCCTGAAGATTTTGCAG |
| | | | TGTATTACTGTCAGCAGTATGGCAGTTCACCTCGGACGTTCGGCCAAGGGACCAAGGTGGAAAT |
| | | | CAAA |
| BGA-6938 | 11 | HCDR1 (Kabat) | TSNYWN |
| | 2 | HCDR2 (Kabat) | EIYPTGNTNYSPSLKS |
| | 14 | HCDR3 (Kabat) | GYNWEDP |
| | 23 | LCDR1 (Kabat) | RTSQSINAILLA |
| | 5 | LCDR2 (Kabat) | GASSRAT |
| | 6 | LCDR3 (Kabat) | QQYGSSPRT |
| | 26 | VHAA | QVQLQESGPGLVKPSGTLSLTCAVSGGSISTSNYWNWVRQSPGKGLEWIGEIYPTGNTNYSPSL |
| | | | KSRVTISIDKSKNQFSLKLNSVTAADTAVYYCARGYNWEDPWGQGTLVTVSS |
| | 24 | VL AA | EIVLTQSPGTLSLSPGERATLSCRTSQSINAILLAWYQQKPGQAPRLLIYGASSRATGIPDRES |
| | | | GSGSGTDFTLSISRLEPEDFAVYYCQQYGSSPRTFGQGTKVEIK |
| | 27 | VH DNA | CAGGTGCAGCTGCAGGAGTCGGGCCCAGGACTGGTGAAGCCTTCGGGGACCCTGTCCCTCACCT |
| | | | GCGCTGTCTCTGGTGGCTCCATCAGTACTAGTAACTACTGGAATTGGGTCCGCCAGTCCCCAGG |
| | | | GAAGGGGCTGGAGTGGATTGGAGAAATCTATCCTACTGGGAACACCAACTACAGCCCGTCCCTC |
| | | | AAGAGTCGAGTCACCATATCAATAGACAAGTCCAAGAACCAGTTCTCCCTGAAACTTAATTCTG |
| | | | TGACCGCCGCGGACACGGCCGTGTATTATTGTGCCAGAGGATACAACTGGTTCGACCCCTGGGG |
| | | | CCAGGGAACCCTGGTCACCGTCTCCTCA |
| | 25 | VL DNA | GAAATTGTGTTGACGCAGTCTCCAGGCACCCTGTCTTTGTCTCCAGGGGAAAGAGCCACCCTCT |
| | | | CCTGCAGGACCAGTCAGAGTATTAACGCCATCTTGTTAGCCTGGTACCAGCAGAAACCTGGCCA |
| | | | GGCTCCCAGGCTCCTCATCTATGGTGCATCCAGCAGGGCCACTGGCATCCCCGACAGGTTCAGT |
| | | | GGCAGTGGGTCTGGGACAGACTTCACTCTCAGCATCAGCAGACTGGAGCCTGAAGATTTTGCAG |
| | | | TGTATTACTGTCAGCAGTATGGCAGTTCACCTCGGACGTTCGGCCAAGGGACCAAGGTGGAAAT |
| | | | CAAA |
| BGA-5488 | 11 | HCDR1 (Kabat) | TSNYWN |
| | 2 | HCDR2 (Kabat) | EIYPTGNTNYSPSLKS |
| | 28 | HCDR3 (Kabat) | GYNYFDP |
| | 23 | LCDR1 (Kabat) | RTSQSINAILLA |
| | 5 | LCDR2 (Kabat) | GASSRAT |
| | 6 | LCDR3 (Kabat) | QQYGSSPRT |
| | 29 | VH AA | QVQLQESGPGLVKPSGTLSLTCAVSGGSISTSNYWNWVRQSPGKGLEWIGEIYPTGNTNYSPSL |
| | | | KSRVTISIDKSKNQFSLKLNSVTAADTAVYYCARGYNYFDPWGQGTLVTVSS |
| | 24 | VL AA | EIVLTQSPGTLSLSPGERATLSCRTSQSINAILLAWYQQKPGQAPRLLIYGASSRATGIPDRES |
| | | | GSGSGTDFTLSISRLEPEDFAVYYCQQYGSSPRTFGQGTKVEIK |
| | 30 | VH DNA | CAGGTGCAGCTGCAGGAGTCGGGCCCAGGACTGGTGAAGCCTTCGGGGACCCTGTCCCTCACCT |
| | | | GCGCTGTCTCTGGTGGCTCCATCAGTACTAGTAACTACTGGAATTGGGTCCGCCAGTCCCCAGG |
| | | | GAAGGGGCTGGAGTGGATTGGAGAAATCTATCCTACTGGGAACACCAACTACAGCCCGTCCCTC |
| | | | AAGAGTCGAGTCACCATATCAATAGACAAGTCCAAGAACCAGTTCTCCCTGAAACTTAATTCTG |
| | | | TGACCGCCGCGGACACGGCCGTGTATTATTGTGCCAGAGGATACAACTACTTCGACCCCTGGGG |
| | | | CCAGGGAACCCTGGTCACCGTCTCCTCA |
| | 25 | VL DNA | GAAATTGTGTTGACGCAGTCTCCAGGCACCCTGTCTTTGTCTCCAGGGGAAAGAGCCACCCTCT |
| | | | CCTGCAGGACCAGTCAGAGTATTAACGCCATCTTGTTAGCCTGGTACCAGCAGAAACCTGGCCA |
| | | | GGCTCCCAGGCTCCTCATCTATGGTGCATCCAGCAGGGCCACTGGCATCCCCGACAGGTTCAGT |
| | | | GGCAGTGGGTCTGGGACAGACTTCACTCTCAGCATCAGCAGACTGGAGCCTGAAGATTTTGCAG |
| | | | TGTATTACTGTCAGCAGTATGGCAGTTCACCTCGGACGTTCGGCCAAGGGACCAAGGTGGAAAT |
| | | | CAAA |
| IgG1mf | 31 | AA | ASTKGPSVFPLAPSSKSTSGGTAALGCLVKDYFPEPVTVSWNSGALTSGVHTFPAVLQSSGLYS |
| | | | LSSVVTVPSSSLGTQTYICNVNHKPSNTKVDKKVEPKSCDKTHTCPPCPAPPAAGPSVELFPPK |
| | | | PKDTLMISRTPEVTCVVVDVSHEDPEVKENWYVDGVEVHNAKTKPREEQYNSTYRVVSVLTVLH |
| | | | QDWLNGKEYKCKVSNKALAAPIEKTISKAKGQPREPQVYTLPPSRDELTKNQVSLTCLVKGFYP |
| | | | SDIAVEWESNGQPENNYKTTPPVLDSDGSFFLYSKLTVDKSRWQQGNVFSCSVMHEALHNHYTQ |
| | | | KSLSLSPGK |
| Heavy chain | 33 | AA | ASTKGPSVFPLAPSSKSTSGGTAALGCLVKDYFPEPVTVSWNSGALTSGVHTEPAVLQSSGLYS |
| constant | | | LSSVVTVPSSSLGTQTYICNVNHKPSNTKVDKKVEPKSCDKTHTCPPCPAPELLGGPSVFLFPP |
| region from | | | KPKDTLMISRTPEVTCVVVDVSHEDPEVKENWYVDGVEVHNAKTKPREEQYNSTYRVVSVLTVL |
| IgG1 | | | HQDWLNGKEYKCKVSNKALPAPIEKTISKAKGQPREPQVYTLPPSRDELTKNQVSLTCLVKGFY |
| | | | PSDIAVEWESNGQPENNYKTTPPVLDSDGSFFLYSKLTVDKSRWQQGNVFSCSVMHEALHNHYT |
| | | | QKSLSLSPGK |
| Light chain | 34 | AA | RTVAAPSVEIFPPSDEQLKSGTASVVCLLNNFYPREAKVQWKVDNALQSGNSQESVTEQDSKDS |
| constant | | | TYSLSSTLTLSKADYEKHKVYACEVTHQGLSSPVTKSENRGEC |
| region of | | | |
| kappa chain | | | |
| VH of | 78 | AA | QVQLQESGPGLVKPSETLSLTCTVSGESLTSYGVHWIRQPPGKGLEWIGVIYADGSTNYNPSLK |
| Tislelizumab | | | SRVTISKDTSKNQVSLKLSSVTAADTAVYYCARAYGNYWYIDVWGQGTTVTVSS |
| VL of | 79 | AA | DIVMTQSPDSLAVSLGERATINCKSSESVSNDVAWYQQKPGQPPKLLINYAFHRFTGVPDRESG |
| Tislelizumab | | | SGYGTDFTLTISSLQAEDVAVYYCHQAYSSPYTFGQGTKLEIK |
| Antibody | 300 | HCDR1 (Kabat) | NYDIN |
| BGA-3295 | 400 | HCDR2 (Kabat) | WIYPGDGSAYYNEKFKG |
| | 500 | HCDR3 (Kabat) | WGYGGVPFAY |
| | 600 | LCDR1 (Kabat) | KSSQSLLNDINQKNYLA |
| | 700 | LCDR2 (Kabat) | FASTGVS |
| | 800 | LCDR3 (Kabat) | QQYYSFPWT |
| | 900 | VH AA | QIQVVQSGPELVKPGSLVKISCKASGYTFTNYDINWVKQRPGQGLEWIGWIYPGDGSAYYNEKF |
| | | | KGKATLTADKSSSTAYMQLSSLTSENSAVYFCARWGYGGVPFAYWGQGTLVTVSA |
| | 1000 | VL AA | DIVMTQSPSSLAMSVGQKVTMSCKSSQSLLNDINQKNYLAWYQQKPGQSPKLLVYFASTGVSGV |
| | | | PDRFIGSGSGTDFTLTVSSVQAEDLADYFCQQYYSFPWTFGGGTKLEIK |
| | 1100 | VH DNA | CAGATCCAAGTIGTGCAGTCTGGACCTGAGCTGGTGAAGCCTGGGAGTTTAGTGAAGATATCCT |
| | | | GCAAGGCTTCTGGTTACACCTTCACAAACTACGATATAAACTGGGTGAAGCAGAGGCCTGGACA |
| | | | GGGACTTGAGTGGATTGGATGGATTTATCCTGGAGATGGTAGTGCTTACTACAATGAGAAGTTC |
| | | | AAGGGCAAGGCCACACTGACTGCAGACAAATCCTCCAGCACAGCCTACATGCAGCTCAGCAGTC |
| | | | TGACTTCTGAGAACTCTGCAGTCTATTTCTGTGCAAGATGGGGTTACGGGGGGGTCCCGTTTGC |
| | | | TTACTGGGGCCAAGGGACTCTGGTCACTGTCTCTGCA |
| | 1200 | VL DNA | GACATTGTGATGACACAGTCTCCATCCTCCCTGGCTATGTCAGTAGGACAGAAGGTCACTATGA |
| | | | GCTGCAAGTCCAGTCAGAGCCTTTTAAATGATATCAATCAAAAGAACTATTTGGCCTGGTACCA |
| | | | GCAGAAACCAGGACAGTCTCCTAAACTTCTTGTATACTTTGCATCCACTGGGGTATCTGGGGTC |
| | | | CCTGATCGCTTCATAGGCAGTGGATCTGGGACAGATTTCACTCTTACCGTCAGCAGTGTGCAGG |
| | | | CTGAAGACCTGGCAGATTACTTCTGTCAGCAATATTATAGTTTTCCGTGGACGTTCGGTGGAGG |
| | | | CACCAAGCTAGAAATCAAA |
| BGA-4348 | 300 | HCDR1 (Kabat) | NYDIN |
| | 400 | HCDR2 (Kabat) | WIYPGDGSAYYNEKEKG |
| | 500 | HCDR3 (Kabat) | WGYGGVPFAY |
| | 600 | LCDR1 (Kabat) | KSSQSLLNDINQKNYLA |
| | 700 | LCDR2 (Kabat) | FASTGVS |
| | 800 | LCDR3 (Kabat) | QQYYSFPWT |
| | 1300 | VH AA | QIQLVQSGAEVKKPGSSVKVSCKASGYTFTNYDINWVRQAPGQGLEWMGWIYPGDGSAYYNEKF |
| | | | KGRATITADKSTSTAYMELSSLRSEDTAVYYCARWGYGGVPFAYWGQGTLVTVSS |
| | 1400 | VL AA | DIVMTQSPDSLAVSLGERATINCKSSQSLLNDINQKNYLAWYQQKPGQSPKLLVYFASTGVSGV |
| | | | PDRFSGSGSGTDFTLTISSLQAEDVAVYYCQQYYSFPWTFGGGTKVEIK |
| | 1500 | VH DNA | CAGATTCAGCTGGTGCAGAGCGGCGCCGAGGTGAAGAAACCCGGCTCCTCCGTGAAAGTCAGCT |
| | | | GCAAGGCCAGCGGCTACACCTTCACCAACTACGACATCAACTGGGTGAGGCAAGCTCCCGGCCA |
| | | | AGGACTGGAGTGGATGGGATGGATCTACCCCGGCGACGGCAGCGCCTACTACAACGAGAAGTTC |
| | | | AAGGGAAGAGCCACCATCACCGCCGACAAGAGCACCAGCACAGCCTACATGGAGCTGAGCTCTC |
| | | | TGAGGTCCGAGGACACCGCCGTGTACTATTGCGCCAGATGGGGCTATGGCGGCGTGCCTTTCGC |
| | | | CTATTGGGGCCAAGGCACACTGGTGACAGTGAGCAGC |
| | 1600 | VL DNA | GACATCGTGATGACCCAGAGCCCCGACTCTCTGGCTGTGTCTCTGGGCGAGAGAGCCACCATCA |
| | | | ACTGCAAGAGCAGCCAGTCTCTGCTGAACGACATCAACCAGAAGAACTATCTGGCTTGGTACCA |
| | | | GCAGAAGCCCGGCCAGAGCCCTAAGCTGCTGGTGTACTTTGCCAGCACCGGCGTCAGCGGAGTG |
| | | | CCCGATAGGTTTTCCGGCAGCGGCTCCGGCACAGACTTTACACTGACCATCTCCTCTCTGCAAG |
| | | | CCGAGGACGTGGCTGTGTACTACTGCCAGCAGTACTACTCCTTCCCTTGGACCTTCGGCGGCGG |
| | | | CACAAAGGTGGAGATTAAG |
| | 300 | HCDR1 (Kabat) | NYDIN |
| BGA-5063 | 1700 | HCDR2 (Kabat) | WIYPGDASAYYNEKEKG |
| | 500 | HCDR3 (Kabat) | WGYGGVPFAY |
| | 600 | LCDR1 (Kabat) | KSSQSLLNDINQKNYLA |
| | 700 | LCDR2 (Kabat) | FASTGVS |
| | 800 | LCDR3 (Kabat) | QQYYSEPWT |
| | 1800 | VH | QVQLVQSGAEVKKPGSSVKVSCKASGYTFTNYDINWVRQAPGQGLEWMGWIYPGDASAYYNEKE |
| | | AA | KGRATITADKSTSTAYMELSSLRSEDTAVYYCARWGYGGVPFAYWGQGTLVTVSS |
| | 1400 | VL | DIVMTQSPDSLAVSLGERATINCKSSQSLLNDINQKNYLAWYQQKPGQSPKLLVYFASTGVSGV |
| | | AA | PDRFSGSGSGTDETLTISSLQAEDVAVYYCQQYYSFPWTFGGGTKVEIK |
| | 1900 | VH | CAGGTGCAGCTGGTGCAGAGCGGCGCCGAGGTGAAGAAACCCGGCTCCTCCGTGAAAGTCAGCT |
| | | DNA | GCAAGGCCAGCGGCTACACCTTCACCAACTACGACATCAACTGGGTGAGGCAAGCTCCCGGCCA |
| | | | AGGACTGGAGTGGATGGGATGGATCTACCCCGGCGACGCCAGCGCCTACTACAACGAGAAGTTC |
| | | | AAGGGAAGAGCCACCATCACCGCCGACAAGAGCACCAGCACAGCCTACATGGAGCTGAGCTCTC |
| | | | TGAGGTCCGAGGACACCGCCGTGTACTATTGCGCCAGATGGGGCTATGGCGGCGTGCCTTTCGC |
| | | | CTATTGGGGCCAAGGCACACTGGTGACAGTGAGCAGC |
| | 1600 | VL | GACATCGTGATGACCCAGAGCCCCGACTCTCTGGCTGTGTCTCTGGGCGAGAGAGCCACCATCA |
| | | DNA | ACTGCAAGAGCAGCCAGTCTCTGCTGAACGACATCAACCAGAAGAACTATCTGGCTTGGTACCA |
| | | | GCAGAAGCCCGGCCAGAGCCCTAAGCTGCTGGTGTACTTTGCCAGCACCGGCGTCAGCGGAGTG |
| | | | CCCGATAGGTTTTCCGGCAGCGGCTCCGGCACAGACTTTACACTGACCATCTCCTCTCTGCAAG |
| | | | CCGAGGACGTGGCTGTGTACTACTGCCAGCAGTACTACTCCTTCCCTTGGACCTTCGGCGGCGG |
| | | | CACAAAGGTGGAGATTAAG |
| Full-length | 801 | AA | MLRRRGSPGMGVHVGAALGALWFCLTGALEVQVPEDPVVALVGTDAT |
| human | | | LCCSFSPEPGFSLAQLNLIWQLTDTKQLVHSFAEGQDQGSAYANRTALF |
| B7H3 | | | PDLLAQGNASLRLQRVRVADEGSFTCFVSIRDFGSAAVSLQVAAPYSKP |
| | | | SMTLEPNKDLRPGDTVTITCSSYQGYPEAEVFWQDGQGVPLTGNVTTSQ |
| | | | MANEQGLFDVHSILRVVLGANGTYSCLVRNPVLQQDAHSSVTITPQRSP |
| | | | TGAVEVQVPEDPVVALVGTDATLRCSFSPEPGFSLAQLNLIWQLTDTKQ |
| | | | LVHSFTEGRDQGSAYANRTALFPDLLAQGNASLRLQRVRVADEGSFTCF |
| | | | VSIRDFGSAAVSLQVAAPYSKPSMTLEPNKDLRPGDTVTITCSSYRGYPE |
| | | | AEVFWQDGQGVPLTGNVTTSQMANEQGLFDVHSVLRVVLGANGTYSC |
| | | | LVRNPVLQQDAHGSVTITGQPMTFPPEALWVTVGLSVCLIALLVALAFV |
| | | | CWRKIKQSCEEENAGAEDQDGEGEGSKTALQPLKHSDSKEDDGQEIA |

### Example 8. Assay and In Vitro Data

### Cell Line Information

### NCI-H1650 (ATCC, CRL-5883)

NCI-H1650 is a cell line exhibiting epithelial morphology that was isolated in 1987 from the lung tissue of a 27-year-old male smoker with stage 3B bronchoalveolar carcinoma, and NCI-H1650 was purchased from ATCC. The base medium for NCI-H1650 is ATCC-formulated RPMI-1640 Medium, ATCC 30-2001. To make the complete growth medium, fetal bovine serum was added to the base medium to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

### NCI-H1048 (ATCC, CRL-5853)

NCI-H1048 is a cell line exhibiting epithelial morphology, and NCI-H1048 was purchased from ATCC. The base medium for NCI-H1048 is ATCC-formulated DMEM:F12 Medium Catalog No. 30-2006. To make the complete growth medium, fetal bovine serum was added to the base medium to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

### Capan-1 (ATCC, HTB-79)

Capan-1 is a cell line with epithelial morphology that was isolated from the pancreas of a 40-year-old white male with pancreatic adenocarcinoma, and Capan-1 was purchased from ATCC. The base medium for Capan-1 is ATCC-formulated Iscove's Modified Dulbecco's Medium, Catalog No. 30-2005. To make the complete growth medium, fetal bovine serum was added to the base medium to a final concentration of 20% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

### MDA-MB-453 (SIBS)

MDA-MB-453 was derived from an effusion of a 48-year-old female patient with metastatic carcinoma of the breast, involving the nodes, brain, and both pleural and pericardial cavities, and MDA-MB-453 was purchased from SIBS. The base medium for MDA-MB-453 is RPMI 1640 Medium, -LEPES (Gibco, 22400105). To make the complete growth medium, fetal bovine serum was added to the base medium to a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

TABLE 5
Cell lines: B7H3 expression level

| Cell lines | Molecular No. (QSC kit) |
|---|---|
| NCI-H1650 | 505998 |
| NCI-H1048 | 100687 |
| Capan-1 | 63062 |
| MDA-MB-453 | 0 |

### Example 8a. ADC Direct Killing in NCI-H1650, Capan-1, NCI-H1048, and MDA-MB-453 Cancer Lines

### Method: ADC Direct Killing

NCI-H1650, NCI-H1048, MDA-MB-453 (2E3/well) or Capan-1 (4E3/well) cells were seeded into 3D-96-well plates (Corning: 4520) at 80 μl/well and incubated at 37° C., 5% CO₂, overnight.

Fresh growth medium containing the varying concentrations of ADCs was added at 40 pl/well. The cells were incubated at 37° C., 5% CO₂, 6 days.

The cell viability was detected by 3D reagent (Promega, G9683), 100 pd/well. The plates were allowed to incubate at room temperature for 30 minutes to stabilize the luminescent signal. Then the plates were analyzed with a microplate reader.

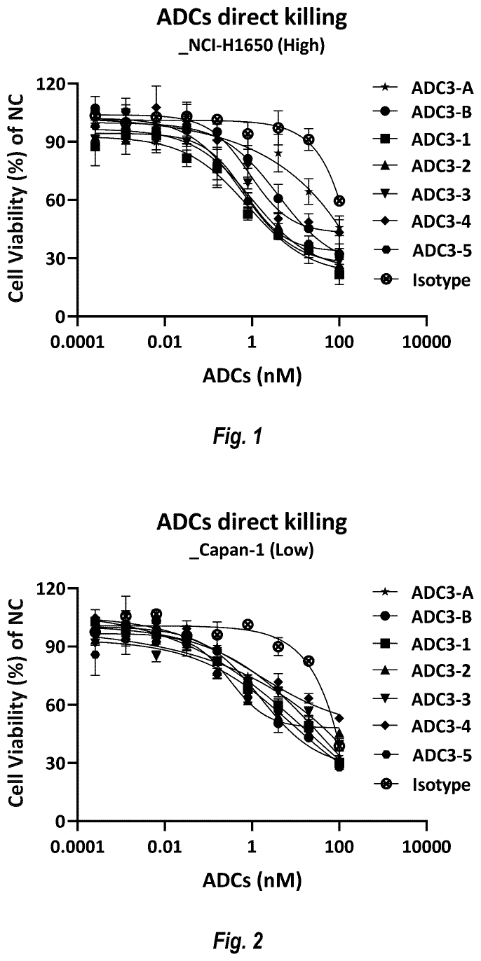

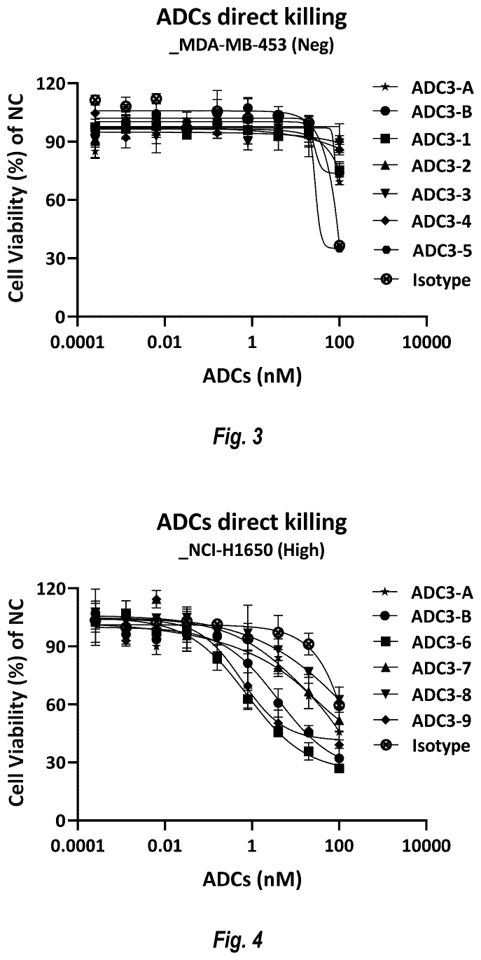

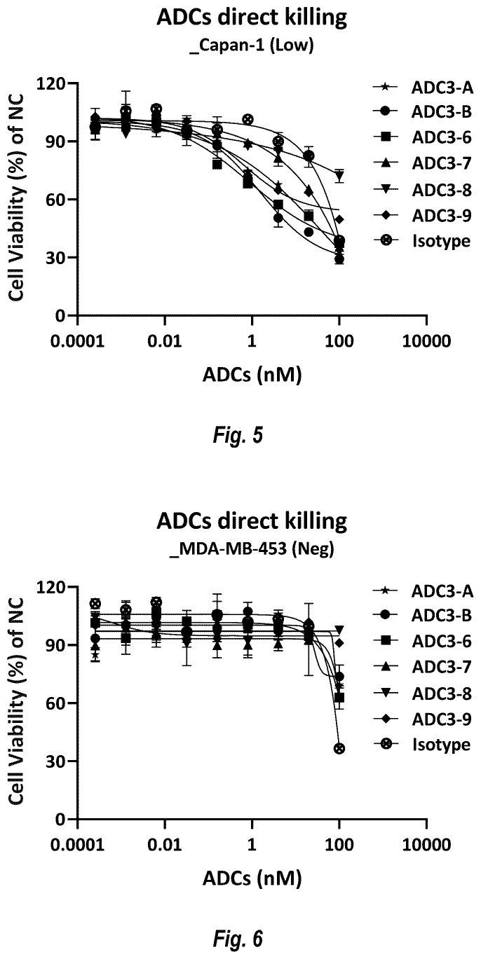

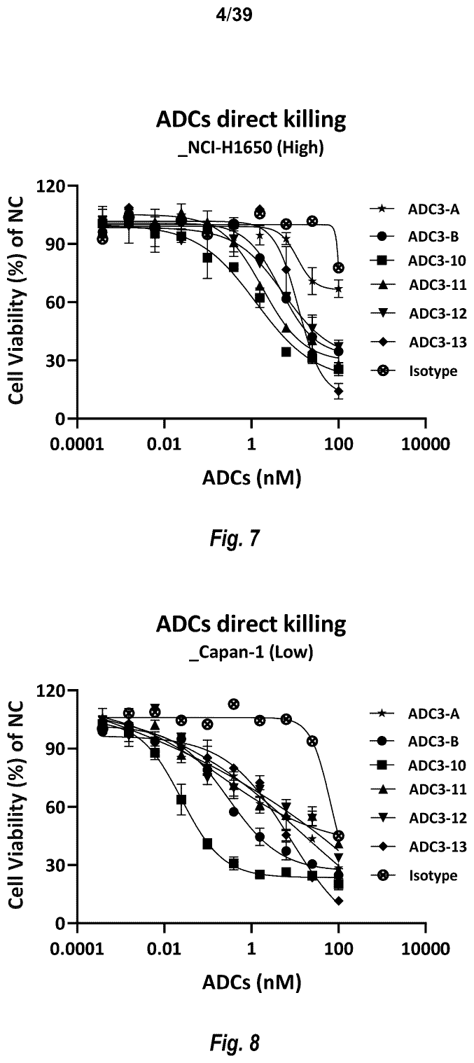

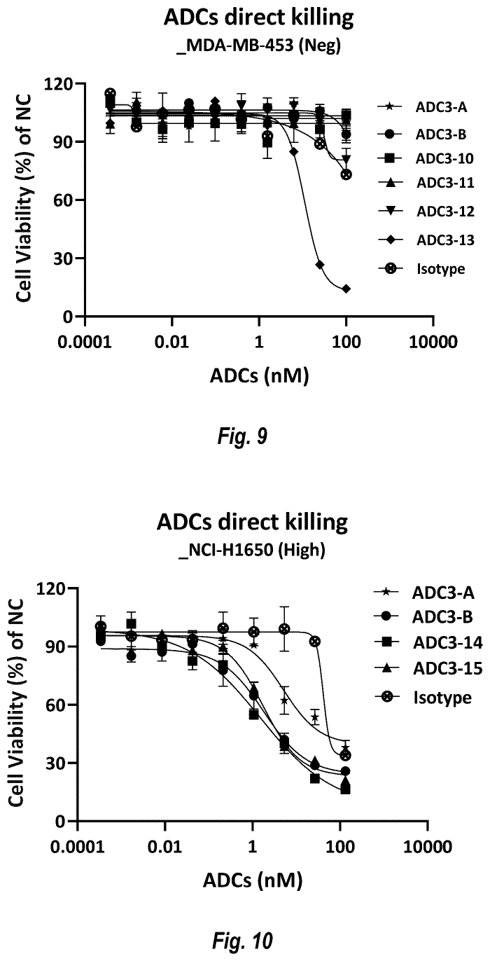

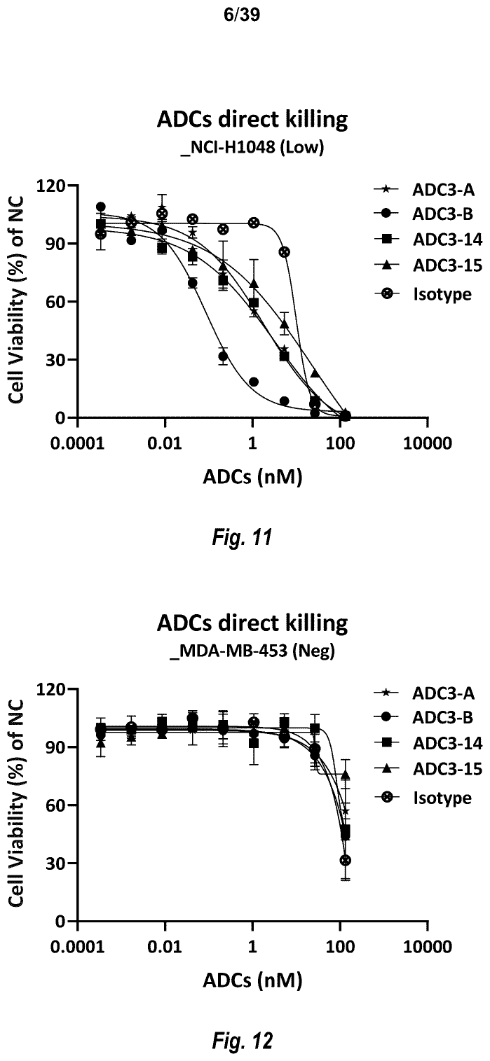

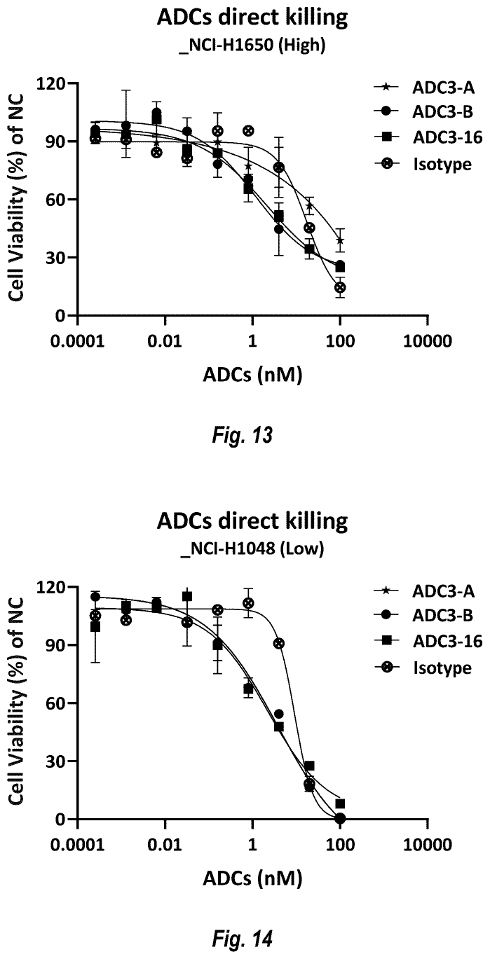

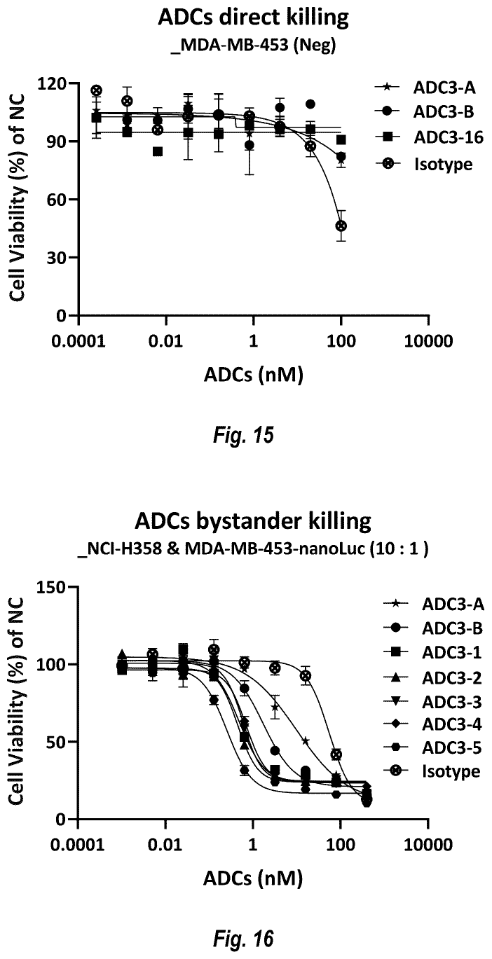

The cellular killing by anti-B7H3 ADCs are shown in Table 6-Table 10, and FIG. 1-FIG. 15.

TABLE 6
ADC direct killing results on B7H3 (+) cell lines

| | NCI-H1650 (High), 3D | | Capan-1 (Low), 3D | |
|---|---|---|---|---|
| ADCs | Emax (%) | EC50 (nM) | Emax (%) | EC50 (nM) |
| ADC3-A | 54.2 | 8.2 | 66.7 | 4.0 |
| ADC3-B | 67.8 | 2.0 | 70.9 | 1.2 |
| ADC3-1 | 78.3 | 0.6 | 69.8 | 1.1 |
| ADC3-2 | 66.4 | 0.5 | 54.4 | 0.3 |
| ADC3-3 | 71.7 | 0.9 | 60.5 | 1.6 |
| ADC3-4 | 56.6 | 1.0 | 47.0 | 0.4 |
| ADC3-5 | 75.4 | 0.5 | 72.3 | 1.7 |

TABLE 7
ADC direct killing results on B7H3 (+) cell lines

| | NCI-H1650 (High), 3D | | Capan-1 (Low), 3D | |
|---|---|---|---|---|
| ADCs | Emax (%) | EC50 (nM) | Emax (%) | EC50 (nM |
| ADC3-A | 54.2 | 8.2 | 66.7 | 4.0 |
| ADC3-B | 67.8 | 2.0 | 70.9 | 1.2 |
| ADC3-6 | 73.1 | 0.6 | 62.5 | 0.6 |
| ADC3-7 | 48.1 | 2.7 | 64.5 | 12.9 |
| ADC3-8 | 37.6 | 6.3 | 28.0 | 2.2 |
| ADC3-9 | 60.9 | 0.4 | 50.2 | 0.6 |

TABLE 8
ADC direct killing results on B7H3 (+) cell lines

| | NCI-H1650 (High), 3D | | Capan-1 (Low), 3D | |
|---|---|---|---|---|
| ADCs | Emax (%) | EC50 (nM) | Emax (%) | EC50 (nM) |
| ADC3-A | 33.1 | 10.9 | 71.4 | 1.7 |
| ADC3-B | 65.4 | 4.5 | 75.1 | 0.3 |
| ADC3-10 | 74.6 | 1.0 | 79.9 | 0.0 |
| ADC3-11 | 72.1 | 2.2 | 58.9 | 0.4 |
| ADC3-12 | 63.1 | 4.1 | 66.4 | 1.6 |
| ADC3-13 | 85.8 | 12.0 | 88.5 | 3.5 |

TABLE 9
ADC direct killing results on B7H3 (+) cell lines

| | NCI-H1650 (High), 3D | | NCI-H1048 (Low), 3D | |
|---|---|---|---|---|
| ADCs | Emax (%) | EC50 (nM) | Emax (%) | EC50 (nM) |
| ADC3-A | 62.0 | 5.7 | 98.9 | 1.2 |
| ADC3-B | 74.2 | 2.0 | 99.4 | 0.1 |
| ADC3-14 | 83.6 | 1.2 | 99.1 | 1.6 |
| ADC3-15 | 78.8 | 2.1 | 97.1 | 4.8 |

TABLE 10
ADC direct killing results on B7H3 (+) cell lines

| | NCI-H1650 (High), 3D | | NCI-H1048 (Low), 3D | |
|---|---|---|---|---|
| ADCs | Emax (%) | EC50 (nM) | Emax (%) | EC50 (nM) |
| ADC3-A | 61.1 | 7.4 | 99.0 | 2.6 |
| ADC3-B | 73.8 | 1.1 | 99.6 | 0.2 |
| ADC3-16 | 75.0 | 1.0 | 92.0 | 1.3 |

### Example 8b. ADC Bystander Killing in NCI-1-1358 Co-Culture with MDA-MB-453-nanoLuc

Method: MDA-MB-453-nanoLuc cell line construction PT67-nanoLuc cells were cultured, then the cell-culture medium (containing the virus (nano-Luc gene)) was collected and filtered. MDA-MB-453 cells were seed in 6-well plates at 1E5 cells/well, and incubated at 37° C., 5% CO₂, overnight. The PT67-nanoLuc cell medium and 8 ug/mli polybrene was added. The infection was repeated 3 times, every one day. Then the MDA-MB-453-nanoLuc cells were cultured with the addition of 1 mg/ml Geneticin for 5 days. The MDA-MB-453-nanoLuc cells were collected, and Nano-Glo reagent (Promega: N1120) was added to test the nano-Luc transfection efficiency.

Method: ADC Bystander Killing NCI-H358 & MDA-MB-453-nanoLuc (10:1), or MDA-MB-453-nanoLuc cels alone were seeded into 3D-96-well plates (Corning: 4520) at 80 μl/well, and Incubated at 37° C., 5% CO₂, overnight. Fresh growth medium containing the varying concentrations of ADCs was added at 40 μl/well. The cells were incubated at 37° C., 5% CO₂, 6 days. The 3D-plates were centrifuged at 1500 rpm, 25° C., 5 in, then the supernatant was discarded.

The Calu-6-nanoLuc cell viability was detected by Nano-Glo reagent (Promega: N1120), 150 μl/well. The 3D-plates were allowed to incubate at room temperature for 10 minutes to stabilize the luminescent signal. Then the plates were analyzed with a microplate reader.

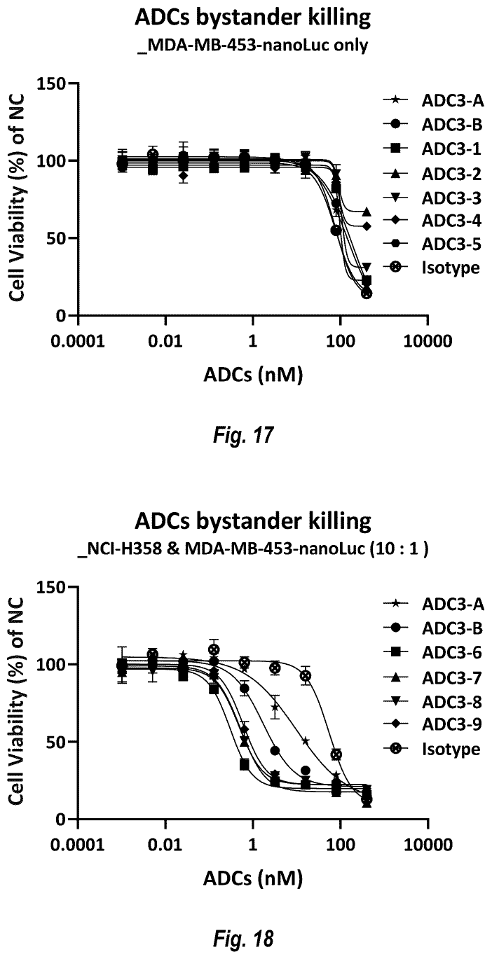

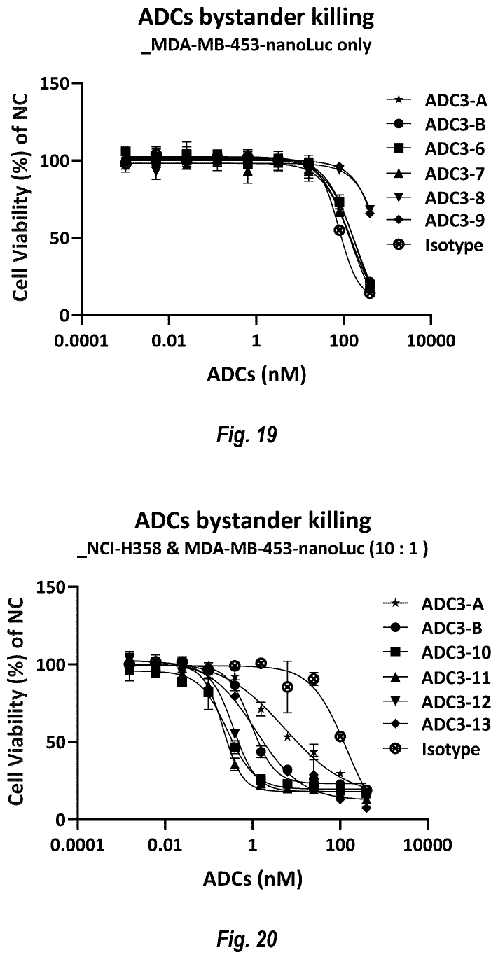

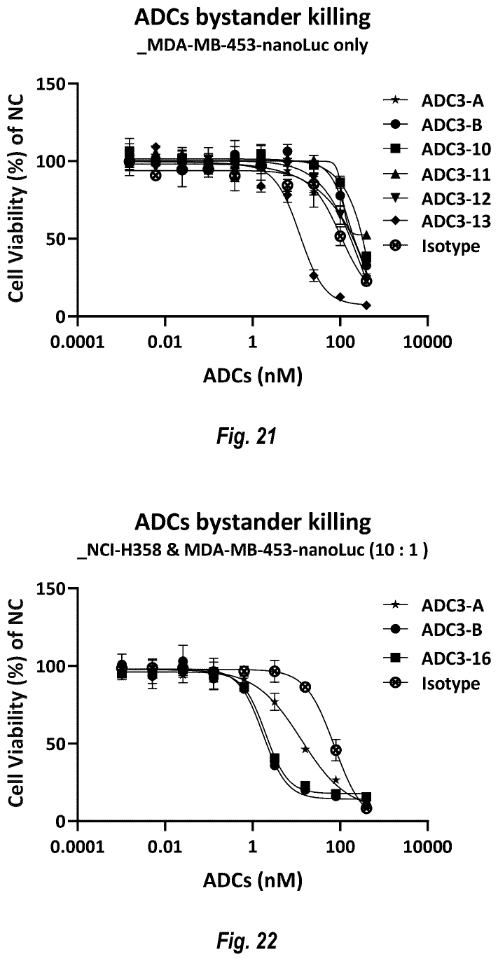

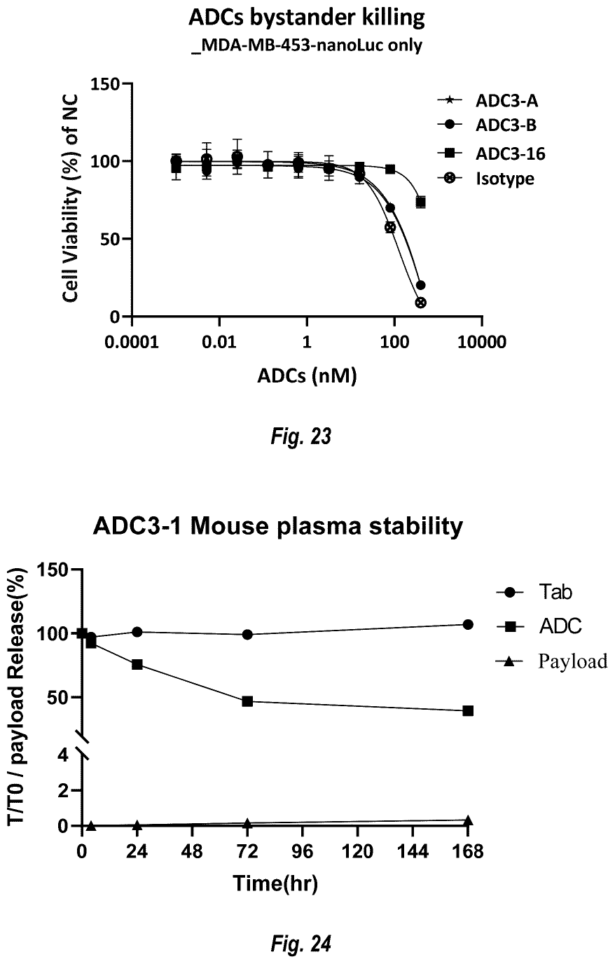

The bystander killing activities of B7H3 ACs are shown in Table 11-Table 14, and FIG. 16-FIG. 23.

TABLE 11
ADC bystander killing effect on NCI-
H358/MDA-MB-453 (nano-Luc) co-culture

NCI-H358:MDA-MB-453-nanoLuc = 10:1, 3D

| ADCs | Emax (%) | EC50 (nM) |
|---|---|---|
| ADC3-A | 89.1 | 9.8 |
| ADC3-B | 79.0 | 1.7 |
| ADC3-1 | 76.4 | 0.5 |
| ADC3-2 | 76.0 | 0.5 |
| ADC3-3 | 75.4 | 0.6 |
| ADC3-4 | 75.4 | 0.7 |
| ADC3-5 | 83.3 | 0.3 |

TABLE 12
ADC bystander killing effect on NCI-
H358/MDA-MB-453 (nano-Luc) co-culture

NCI-H358:MDA-MB-453-nanoLuc = 10:1, 3D

| ADCs | Emax (%) | EC50 (nM) |
|---|---|---|
| ADC3-A | 89.1 | 9.8 |
| ADC3-B | 79.0 | 1.7 |
| ADC3-6 | 80.2 | 0.3 |
| ADC3-7 | 82.3 | 0.5 |
| ADC3-8 | 77.6 | 0.5 |
| ADC3-9 | 77.7 | 0.6 |

TABLE 13
ADC bystander killing effect on NCI-
H358/MDA-MB-453 (nano-Luc) co-culture

NCI-H358:MDA-MB-453-nanoLuc = 10:1, 3D

| ADCs | Emax (%) | EC50 (nM) |
|---|---|---|
| ADC3-A | 82.3 | 5.4 |
| ADC3-B | 83.2 | 1.0 |
| ADC3-10 | 83.2 | 0.3 |
| ADC3-11 | 86.8 | 0.2 |
| ADC3-12 | 86.5 | 0.4 |
| ADC3-13 | 92.9 | 1.5 |

TABLE 14
ADC bystander killing effect on NCI-H358/MDA-
MB-453 (nano-Luc) co-culture assay

NCI-H358:MDA-MB-453-nanoLuc = 10:1, 3D

| ADCs | Emax (%) | EC50 (nM) |
|---|---|---|
| ADC3-A | 89.5 | 13.0 |
| ADC3-B | 89.9 | 1.7 |
| ADC3-16 | 84.3 | 2.0 |

### Example 8c. ADC Plasma Stability Evaluation

Incubation of ADC with Plasma

ADCs were diluted into mouse or human plasma to yield a final solution of 100 μg/mL ADC in plasma. The samples were incubated at 37° C. Aliquots (100 μL) were taken at six time points (0, 4, 24, 72, 96, or 168 h). Samples were frozen at −80° C. until analysis.

Plasma payload concentrations were carried out under the following measurement conditions:
Instrument: LC-MS/MS (Triple Quad 6500 plus)
Monitor: MRM
Column: Advanced Materials Technology, HALO AQ-C18 2.7 μm 90Å, 50*2.1 mm
Column temperature: 40° C.
Mobile phase A: H₂O-0.1% FA
Mobile phase B: ACN-0.1% FA
Gradient program for DXd (1-A) and compounds 1-1˜1-9 2% B-2% B (0 min-0.2 min), 2% B-98% B (0.2 min-1.2 min), 98% B-98% B (1.2 min-2.0 min), 98% B-2% B (2.0 min-2.01 min), 2% B-2% B (2.01 min-4.0 min)

### Injected Sample Amount: 10 μL (DXd and Other Payloads)

Plasma ABC and total Ab (Tab) concentrations were carried out under the following measurement conditions:
Assay: Ligand binding assay (ELISA)
Capture reagent: B7H3 extracellular domain (ECD)
Detection reagent: anti-payload Ab for ADC and anti-human IgG polyclonal Ab for total Ab.

### ADC DAR Changes in Human/Mouse Plasma Stability Study Samples

Method: Human B7H3 ECD was biotinylated and immobilized onto Dynabeads M-280 Streptavidin, and then the ADCs were captured by an ECD-bead system from plasma samples for 2 hours at room temperature. The captured ADCs were then washed with IBS-EP buffer (10 mM Hepes [pH 7.4], 150 mM NaCl, 3.4 mM ethylenediaminetetraacetic acid [EDTA], 0.005% Surfactant P20) and digested using IdeS enzyme at 37° C. for 1 h. After extensive washing of the beads with HBS-EP, water, and 10% acetonitrile, the ADC analytes were eluted using 30% acetonitrile with 1% formic acid. Lastly, reduction was performed for 45 min with 100 mM TCEP. Liquid chromatography-tandem mass spectrometry (LC-MS/MS) method was used for ADC DAR analysis.

### Results

### 1. Mouse and Human Plasma Stability of ADC3-1

Total mAb of ADC3-1 showed good stability while the conjugated ADC was reduced to ˜40% after 168 h incubation in mouse plasma; the recovery of free payload in plasma was -0.3% at the end of incubation (FIG. 24).

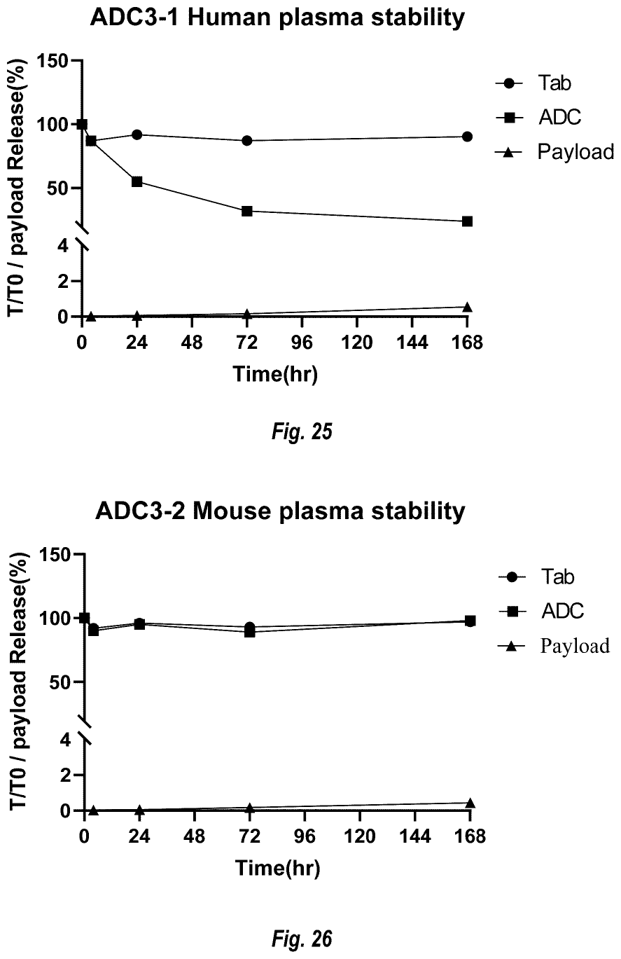

Total mAb of ADC3-1 showed good stability while the conjugated ADC was reduced to ˜25% after 168 h incubation in human plasma; the recovery of free payload in plasma was ˜0.5% at the end of incubation. Average DAR of the ADCs was reduced from 7.34 to 3.40 at the end of incubation (FIG. 25).

### 2. Mouse and Human Plasma Stability of ADC3-2

Total mAb and conjugated ADC of ADC3-2 showed good stability after 168 h incubation in mouse plasma; the recovery of free payload in plasma was -0.3% at the end of incubation (FIG. 26).

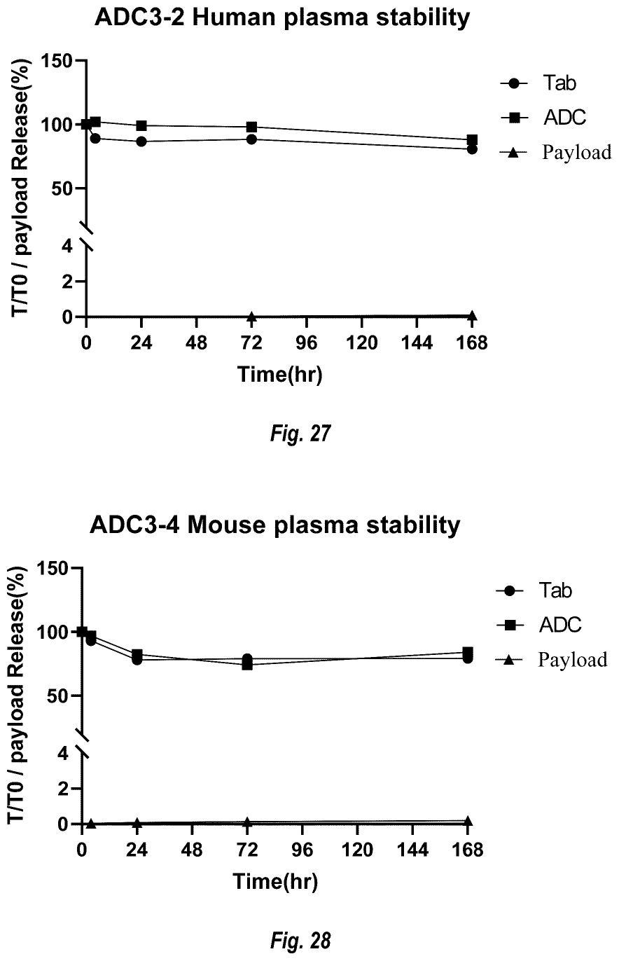

Total mAb and conjugated ADC of ADC3-2 showed good stability after 168 h incubation in human plasma; the recovery of free payload in plasma was -0.1% at the end of incubation. Average DAR of the ADCs was reduced from 7.86 to 7.78 at the end of incubation (FIG. 27).

### 3. Mouse and Human Plasma Stability of ADC3-4

Total mAb and conjugated ADC of ADC3-4 showed good stability after 168 h incubation in mouse plasma; the recovery of free payload in plasma was -0.2% at the end of incubation (FIG. 28).

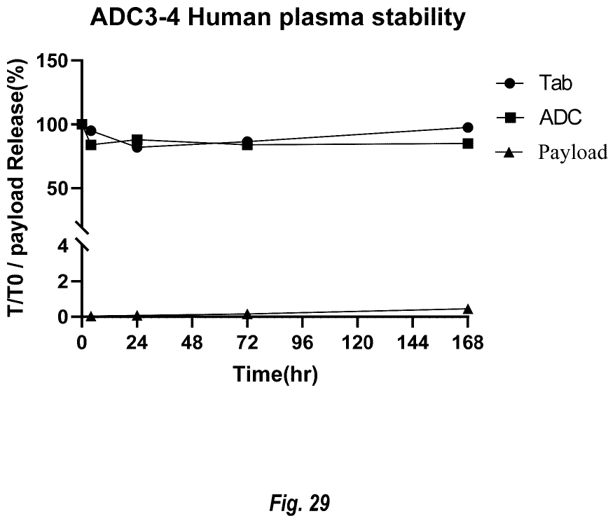

Total mAb and conjugated ADC of ADC3-4 showed good stability after 168 h incubation in human plasma; the recovery of free payload in plasma was ˜0.4% at the end of incubation (FIG. 29Error! Reference source not found.).

### Example 9. ADC In Vivo Efficacy Study

### Methods

Female BALB/c Nude mice were subcutaneously implanted with 3×10⁶ H1650 cells per 200 μL PBS/matrigel in the right flank. After inoculation, tumor volumes were determined twice weekly in two dimensions using a caliper, and were expressed in mm³ using the formula: V=0.5(a×b²) where a and b are the long and short dimensions of the tumor, respectively. When tumors reached a mean volume of approximately 200 mm³, mice were randomly allocated into groups with 8 animals in each group, and were intravenously treated on day 1 with vehicle or ADC as follows: (1) ADC3-A, ADC3-1, ADC3-3, ADC3-14, or ADC3-15 at 3/10 mg/kg; (2) ADC3-A at 10 mg/kg, or ADC3-2, ADC3-4, ADC3-5, or ADC3-6 at 3 mg/kg; (3) ADC3-A at 10 mg/kg, or ADC3-8 or ADC3-9 at 3 mg/kg; (4) ADC3-A at 10 mg/kg, or ADC3-10, ADC3-11, ADC3-12, or ADC3-13 at 3 mg/kg; (5) ADC3-A ADC3-1, ADC3-3, ADC3-14, or ADC3-15 at 3/10 mg/kg.

Partial regression (PR) was defined as tumor volume smaller than 50% of the starting tumor volume on the first day of dosing in three consecutive measurements and complete regression (CR) was defined as tumor volume less than 14 mm³ in three consecutive measurements. Data is presented as mean tumor volume±standard error of the mean (SEM). Tumor growth inhibition (TGI) is calculated using formula (TGI):

$$\% growth inhibition = 100 \times ( 1 - ( \frac{( treated t ) - ( treated to )}{( placebo t ) - ( placebo to )} ) \qquad ( TG 1 )$$

treated t=treated tumor volume at time t
treated t₀=treated tumor volume at time 0
placebo t=placebo tumor volume at time t
placebo t₀=placebo tumor volume at time 0

### Results: (1) ADC3-A, ADC3-1, ADC3-3, ADC3-14, and ADC3-15

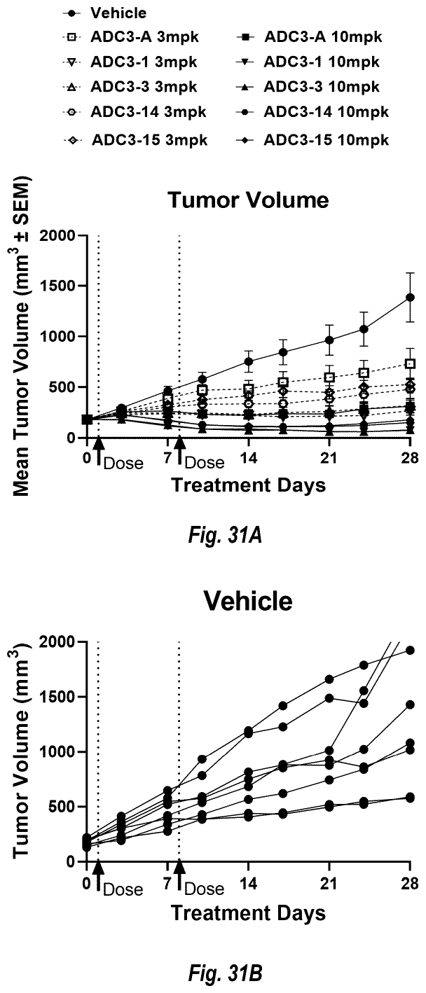

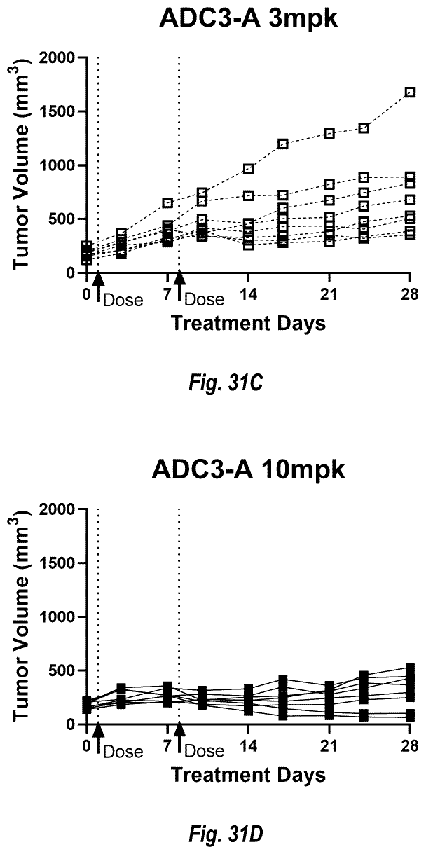

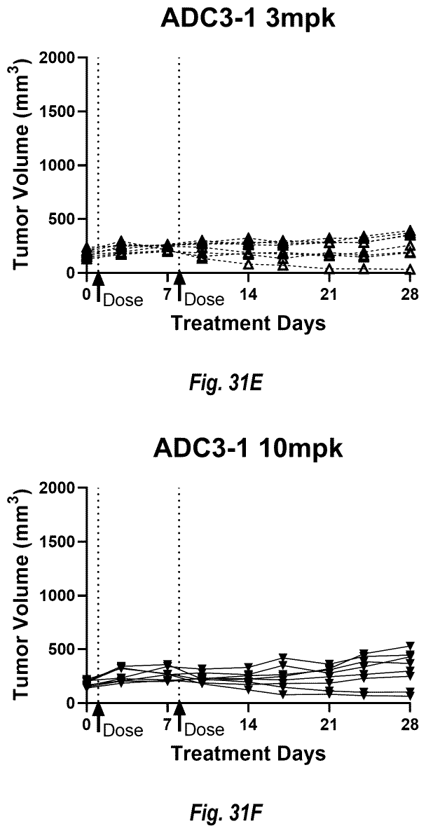

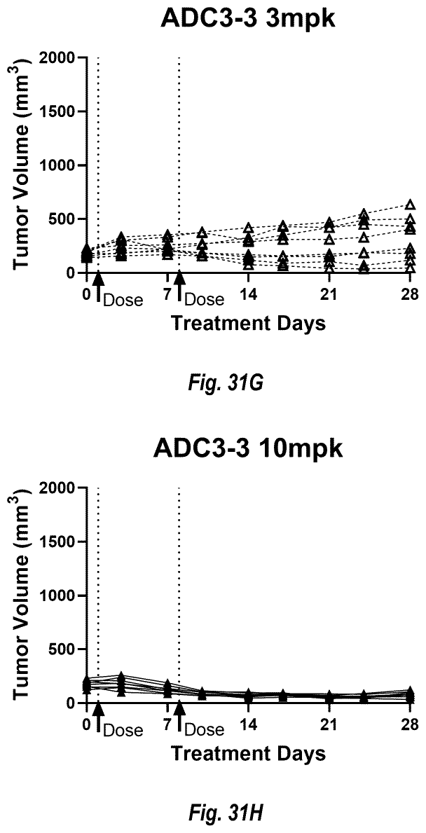

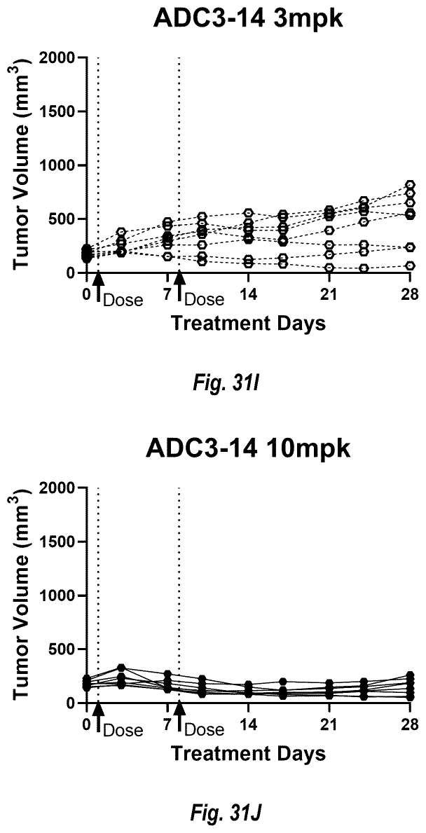

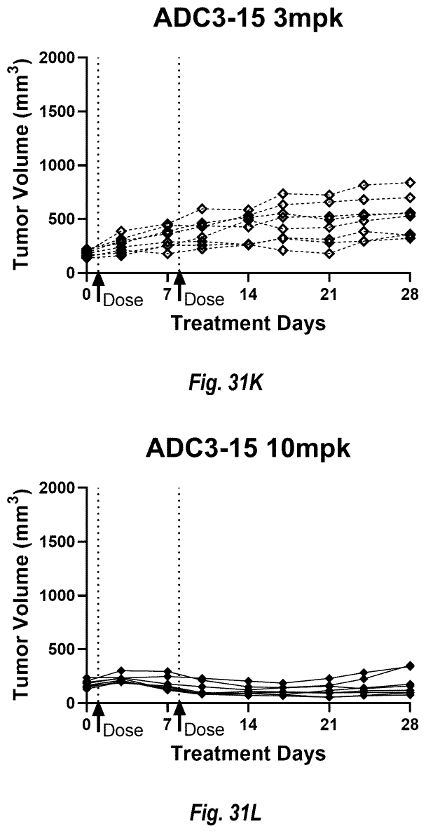

The in vivo efficacies of ADC3-A, ADC3-1, ADC3-3, ADC3-14, and ADC3-15 were compared in H1650 xenografts (B7H3 IHC 3±) grown subcutaneously in BALB/c Nude mice. Treatment with 3/10 mg/kg ADC3-A, 3/10 mg/kg ADC3-1, 3/10 mg/kg ADC3-3, 3/10 mg/kg ADC3-14, or 3/10 mg/kg ADC3-15 resulted in 53%/86%, 90%/104%, 84%/103%, 73%/98%, and 68%/96% TGI on day 28, respectively. ADC3-1 demonstrated better efficacy than ADC3-A at both 3 mg/kg and 10 mg/kg. ADC3-3 demonstrated comparable efficacy to ADC3-A at 3 mg/kg, but better efficacy at 10 mg/kg. ADC3-14 and ADC3-15 demonstrated comparable efficacy to ADC3-A at both 3 mg/kg and 10 mg/kg. All ADCs were well tolerated without any sign of toxicity or significant body weight decrease (FIGS. 31A-31L and Table 15).

TABLE 15
ADC TGI measurement at Day 28.

| ADC | TGI (%) at T 28 |
|---|---|
| ADC3-A 3 mpk | 53 |
| ADC3-A 10 mpk | 86 |
| ADC3-1 3 mpk | 90 |
| ADC3-1 10 mpk | 104 |
| ADC3-3 3 mpk | 84 |
| ADC3-3 10 mpk | 103 |
| ADC3-14 3 mpk | 73 |
| ADC3-14 10 mpk | 98 |
| ADC3-15 3 mpk | 68 |
| ADC3-15 10 mpk | 96 |

### Results: (2) ADC3-A, ADC3-2, ADC3-4, ADC3-5, and ADC3-6

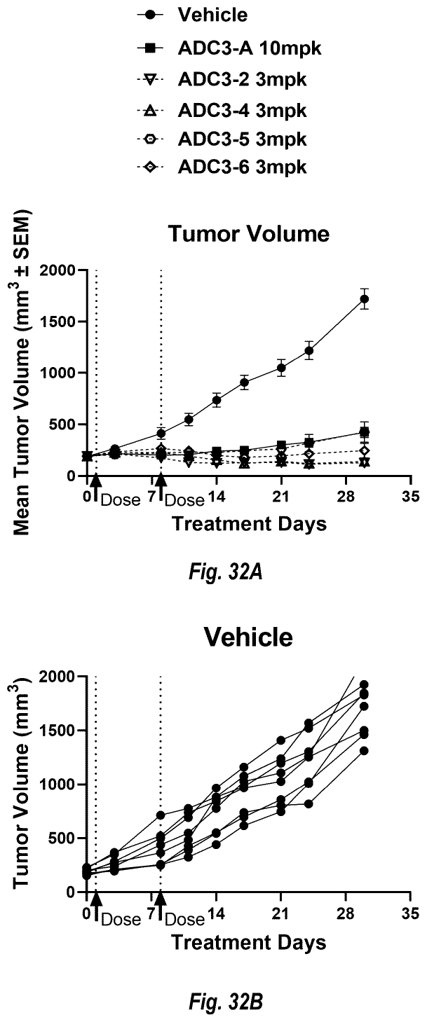

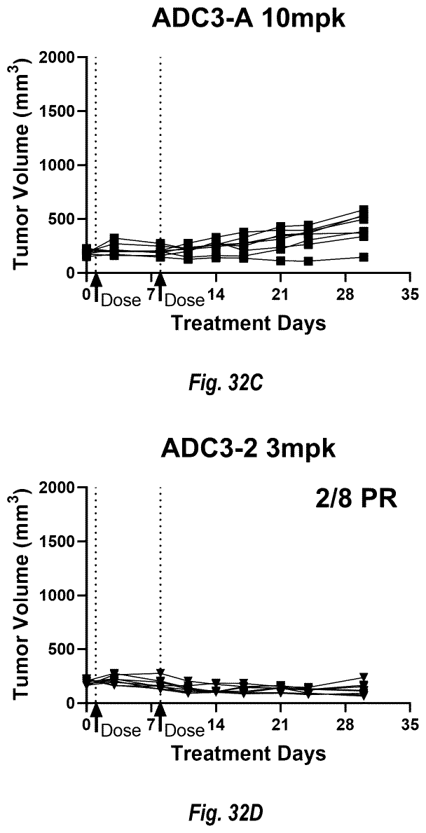

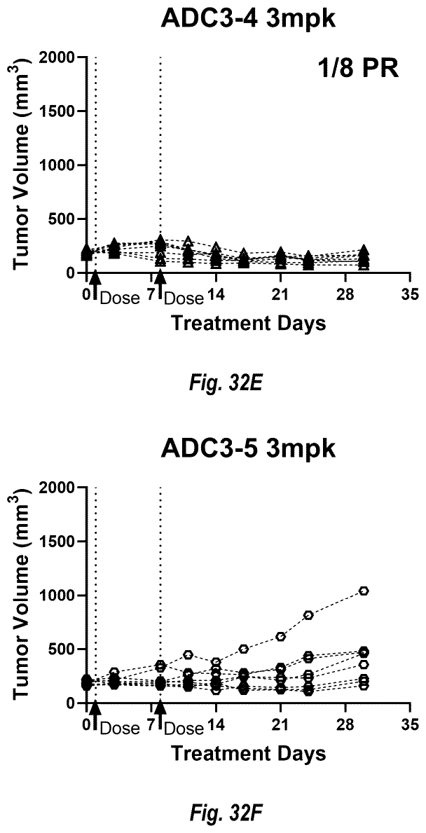

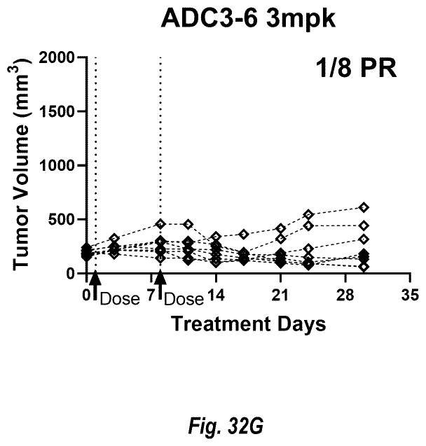

The in vivo efficacies of ADC3-A and ADC3-2, ADC3-4, ADC3-5, and ADC3-6 were compared in H1650 xenografts (B7H3 IHC 3+) grown subcutaneously in BALB/c Nude mice. Treatment with 10 mg/kg ADC3-A or 3 mg/kg ADC3-2, ADC3-4, ADC3-5, or ADC3-6 resulted in 85%, 104%, 103%, 85%, and 96% TGI on day 30, respectively. For individual animal tumor growth inhibition, ADC3-2, ADC3-4, and ADC3-6 induced 2/8 PR, 1/8 PR, and 1/8 PR, respectively. Taken together, ADC3-2 and ADC3-4 at 3 mg/kg demonstrated better efficacy than ADC3-A at 10 mg/kg. ADC3-5 and ADC3-6 at 3 mg/kg demonstrated comparable efficacy to ADC3-A at 10 mg/kg. All ADCs were well tolerated without any sign of toxicity or significant body weight decrease (FIGS. 32A-32G and Table 16).

TABLE 16
ADC TGI measurement at Day 30.

| ADC | TGI (%) at T 30 |
|---|---|
| ADC3-A 10 mpk | 85 |
| ADC3-2 3 mpk | 104 |
| ADC3-4 3 mpk | 103 |
| ADC3-5 3 mpk | 85 |
| ADC3-6 3 mpk | 96 |

### Results: (3) ADC3-A, ADC3-8, and ADC3-9

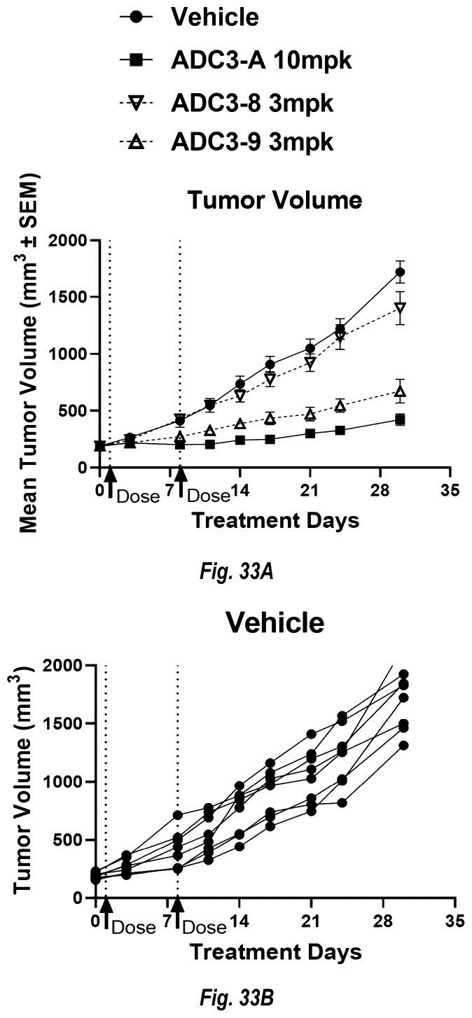

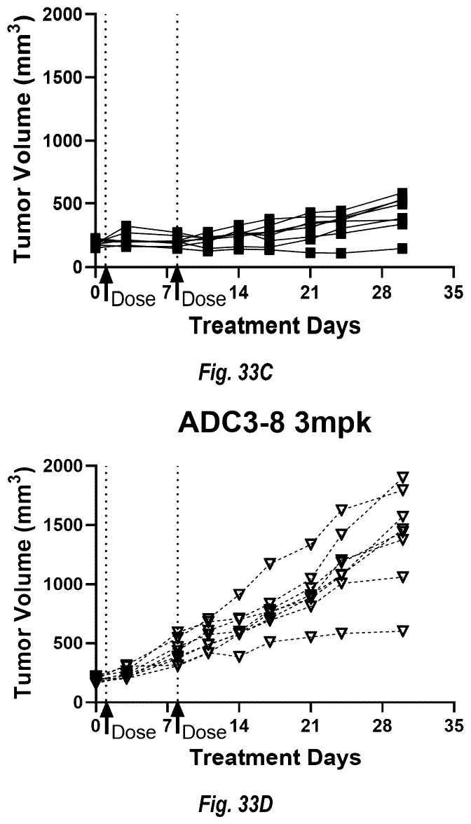

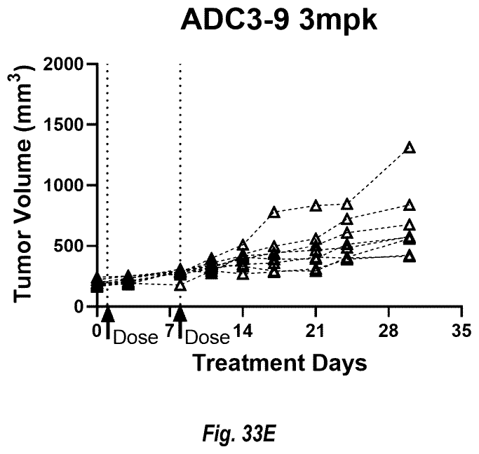

The in vivo efficacies of ADC3-A, ADC3-8, and ADC3-9 were compared in H1650 xenografts (B7H3 HC 3+) grown subcutaneously in BALB/c Nude mice. Treatment with 10 mg/kg ADC3-A, 3 mg/kg ADC3-8, or 3 mg/kg ADC3-9 resulted in 85%, 21%, and 69% TGI on day 30, respectively. ADC3-8 at 3 mg/kg demonstrated weaker efficacy than ADC3-A at 10 mg/kg. ADC3-9 at 3 mg/kg demonstrated comparable efficacy to ADC3-A at 10 mg/kg. All ADCs were well tolerated without any sign of toxicity or significant body weight decrease (FIGS. 33A-33E and Table 17).

TABLE 17
ADC TGI measurement at Day 30.

| ADC | TGI (%) at T 30 |
|---|---|
| ADC3-A 10 mpk | 85 |
| ADC3-8 3 mpk | 21 |
| ADC3-9 3 mpk | 69 |

Results: (4) ADC3-A, ADC3-10, ADC3-11, ADC3-12, and ADC3-13

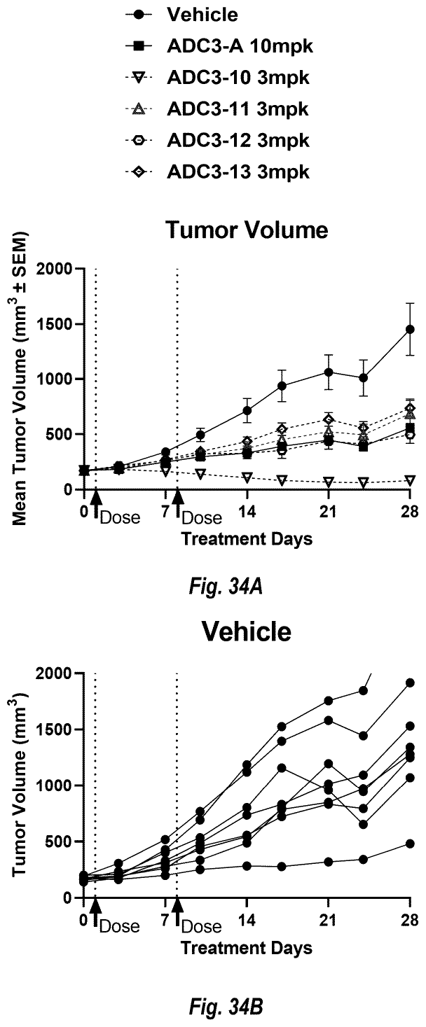

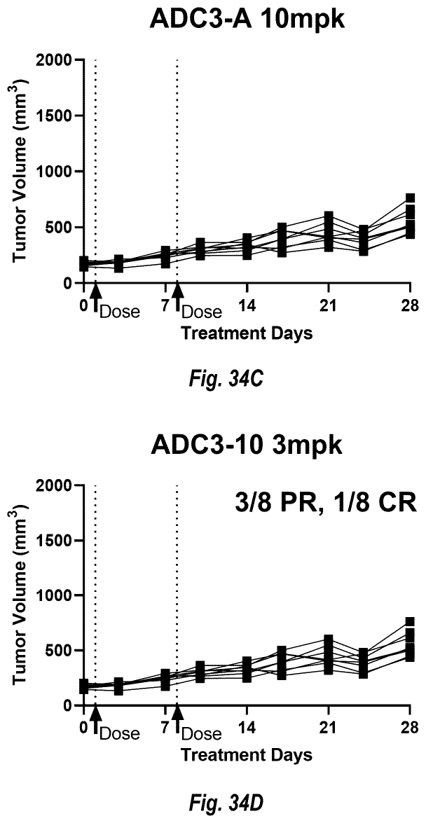

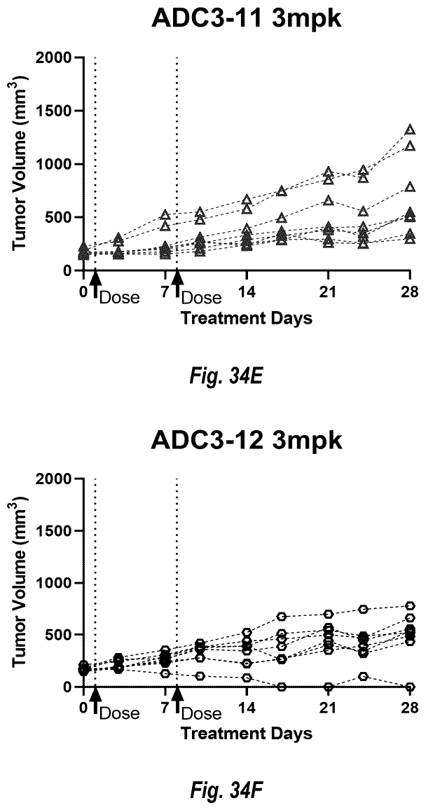

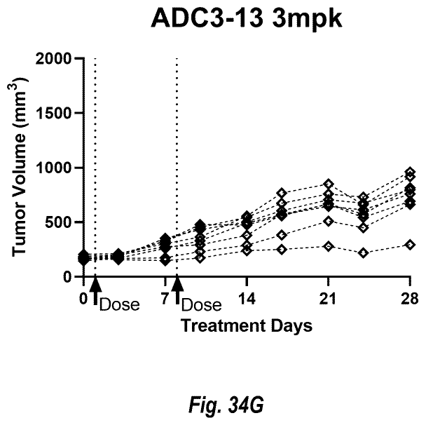

The in vivo efficacies of ADC3-A, ADC3-10, ADC3-11, ADC3-12, and ADC3-13 were compared in H1650 xenografts (B7H3 IHC 3+) grown subcutaneously in BALB/c Nude mice. Treatment with 10 mg/kg ADC3-A or 3 mg/kg ADC3-10, ADC3-11, ADC3-12, or ADC3-13 resulted in 70%, 107%, 60%, 75%, and 56% TGI on day 28, respectively. For individual animal tumor growth inhibition, ADC3-10 induced 3/8 PR and 1/8 CR. Taken together, ADC3-10 at 3 mg/kg demonstrated better efficacy than ADC3-A at 10 mg/kg. ADC3-11, ADC3-1², and ADC3-13 at 3 mg/kg demonstrated comparable efficacy to ADC3-A at 10 mg/kg. All ADCs were well tolerated without any sign of toxicity or significant body weight decrease (FIGS. 34A-34G and Table 18).

TABLE 18
ADC TGI measurement at Day 28

| ADC | TGI (%) at T 28 |
|---|---|
| ADC3-A 10 mpk | 70 |
| ADC3-10 3 mpk | 107 |
| ADC3-11 3 mpk | 60 |
| ADC3-12 3 mpk | 75 |
| ADC3-13 3 mpk | 56 |

### Results: (5) ADC3-A, ADC3-1, ADC3-3, ADC3-14, and ADC3-15

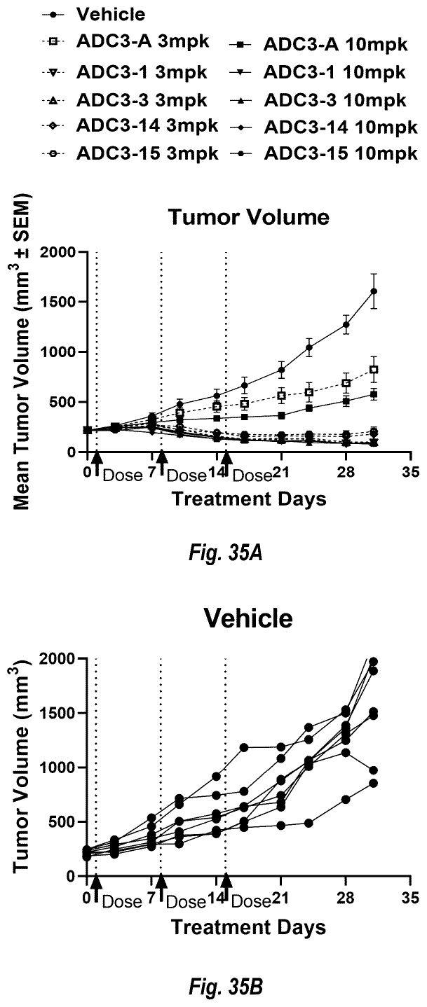

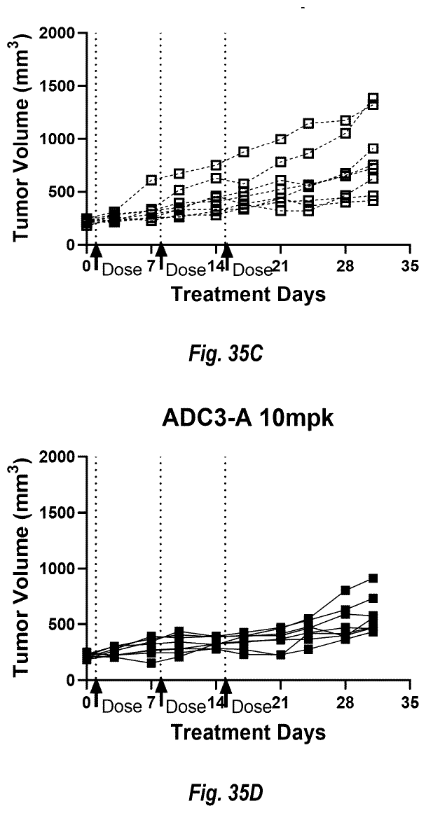

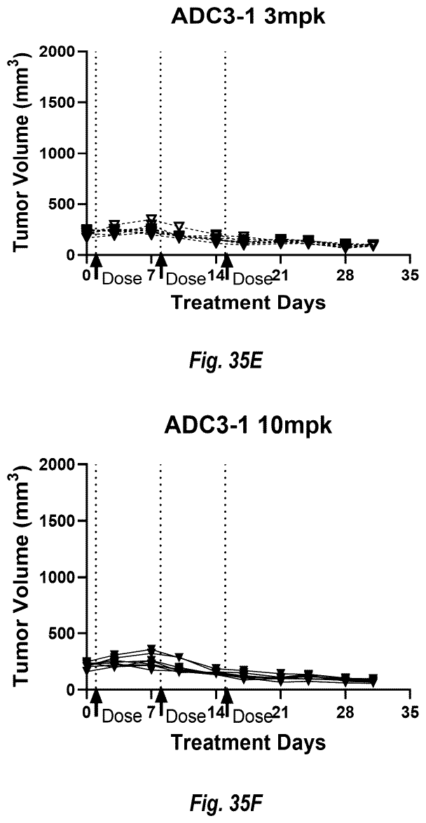

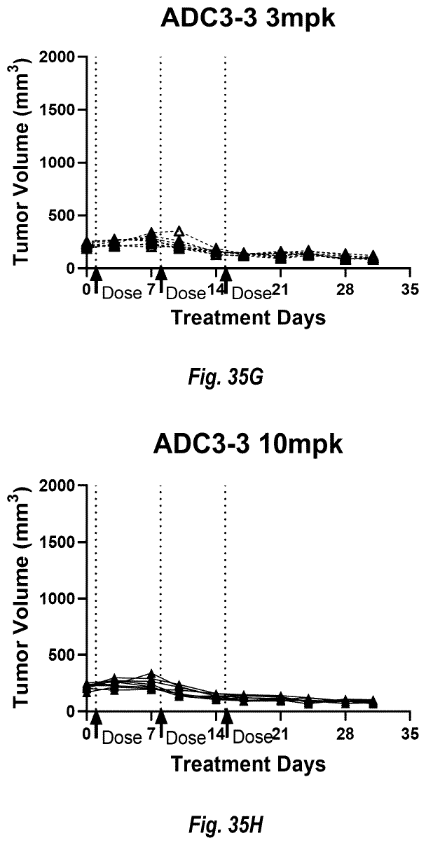

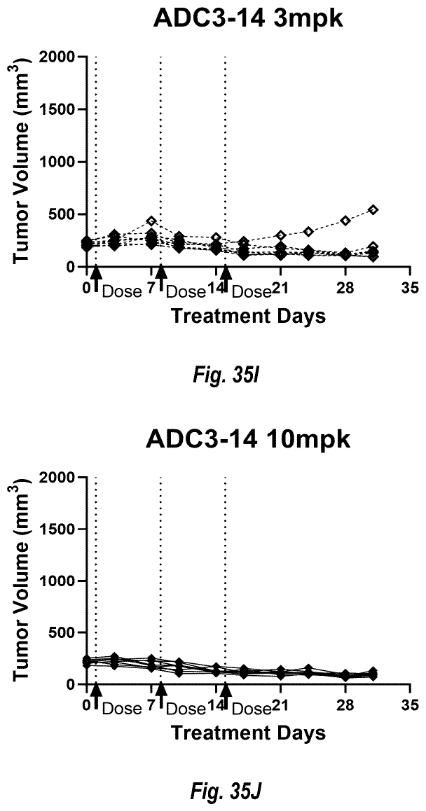

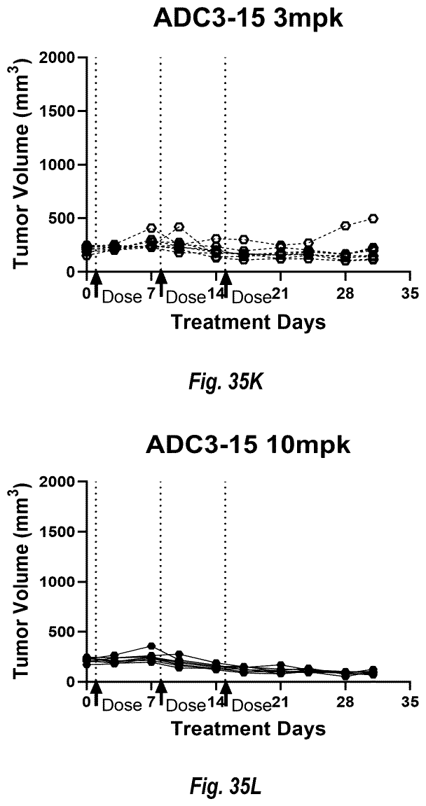

The in vivo efficacies of ADC3-A, ADC3-1, ADC3-3, ADC3-14, and ADC3-15 were compared in H1975 xenografts (B7H3 IHC 3+) grown subcutaneously in BALB/c Nude mice. Treatment with 3/10 mg/kg ADC3-A, 3/10 mg/kg ADC3-1, 3/10 mg/kg ADC3-3, 3/10 mg/kg ADC3-14, or 3/10 mg/kg ADC3-15 resulted in 56%/ 7/4%, 109%/110%, 109%/110%, 103%/109%, and 101%/109% TGI on day 31, respectively. ADC3-1, ADC3-3, ADC3-14, and ADC3-15 demonstrated better efficacy than ADC3-A at both 3 mg/kg and 10 mg/kg. All ADCs were well tolerated without any sign of toxicity or significant body weight decrease (FIGS. 35A-35L and Table 19).

TABLE 19
ADC TGI measurement at Day 31

| ADC | TGI (%) at T 31 |
|---|---|
| ADC3-A 3 mpk | 56 |
| ADC3-A 10 mpk | 74 |
| ADC3-1 3 mpk | 109 |
| ADC3-1 10 mpk | 110 |
| ADC3-3 3 mpk | 109 |
| ADC3-3 10 mpk | 110 |
| ADC3-14 3 mpk | 103 |
| ADC3-14 10 mpk | 109 |
| ADC3-15 3 mpk | 101 |
| ADC3-15 10 mpk | 109 |

### Example 10. Humanization of the Anti-Human B7H3 mAb BGA-3295

For humanization of BGA-3295, human germline IgG genes were searched for sequences that share high degrees of homology to the cDNA sequences of BGA-3295 variable regions by blasting the human immunoglobulin gene database in IMGT and NCBI websites. The human IGVH and IGVκ genes that are present in human antibody repertoires with high frequencies (Glanville 2009 PNAS 106:20216-20221) and are highly homologous to BGA-3295 were selected as the templates for humanization.

Humanization was carried out by CDR-grafting (Methods in Molecular Biology, Vol 248: Antibody Engineering, Methods and Protocols, Humana Press) and the humanized antibodies based on BGA-3295 were engineered as the human IgG1 variant (SEQ ID NO: 31) format using an in-house developed expression vector. In the initial round of humanization, mutations from murine to human amino acid residues in framework regions were guided by the simulated 3D structure, and the murine framework residues of structural importance for maintaining the canonical structures of CDRs were retained in the 1^{st} version of humanized antibody BGA-4348. Specifically, CDRs of BGA-3295 Vκ (SEQ ID NOs: 600-800) were grafted into the framework of human germline variable gene IGVκ4-1 with two murine framework residues retained (S₄₉ and V₅₄) (SEQ ID NOs: 1400 and 1600). CDRs of BGA-3295 Th (SEQ ID NOs: 300-500) were grafted into the framework of human germline variable gene IGVH4-1 with four murine framework residues (I₂, Y₂₇, A₆₈, and K₇₁) residues retained (SEQ ID NOs: 1300 and 1500).

Humanized antibody BGA-4348 was constructed as human full-length antibody format using in-house developed expression vectors that contain constant regions of a human IgG1 variant (SEQ ID NO: 31) and kappa chain, respectively, with easy adapting sub-cloning sites. Expression and preparation of humanized antibodies was achieved by co-transfection of the heavy chain and corresponding light chain constructs into 293G cells (developed in-house) and by purification using a protein A column. The purified antibodies were concentrated to 0.5-5 mg/mL in PBS and stored in aliquots in −80° C. freezer.

Based on BGA-4348, several single mutations were made converting the retained murine residues in the framework region to corresponding human germline residues. Humanized antibodies were also engineered by introducing mutations in CDR regions to remove potential post-translation modification (PTM) sites and improve stability for therapeutic use in humans. All humanization mutations were made using primers containing mutations at specific positions and a site-directed mutagenesis kit (Cat. No. FMI 11-02, TransGen, Beijing, China). The desired mutations were verified by sequencing analysis. These humanized antibodies were tested in binding assays as described elsewhere.

Taken together, the engineered versions of humanized monoclonal antibodies, BGA-4348 (See Table 4), and BGA-5063 (See Table 4), were derived from the mutation process described above, and characterized in detail.

For affinity determination, antibodies were captured by anti-human Fc surface, and used in an affinity assay based on surface plasmon resonance (SPR) technology. The results of SPR-determined binding profiles of humanized antibodies to ECD of human 4Ig-B7H3 (Sinobiological, Cat: 11188-H08H) were summarized in Table 20. BGA-4348 (See Table 4) and BGA-5063 (See Table 4) have comparable binding affinities with dissociation constants at 0.6 nM and 0.9 nM, respectively, which are comparable to that of BGA-3295.

TABLE 20
Comparison of binding affinities of BGA-3295 and
the humanized antibodies thereof to B7H3 by SPR

| | Test 1 | | | Test 2 | | |
|---|---|---|---|---|---|---|
| Ab | kₒₙ (M⁻¹s⁻¹) | k_{off} (s⁻¹) | K_{D} (M) | kₒₙ (M⁻¹s⁻¹) | k_{off} (s⁻¹) | K_{D} (M) |
| Chimeric | 7.20E+05 | 2.84E−04 | 3.94E−010 | — | — | — |
| BGA-3295 | | | | | | |
| BGA-4348 | 3.15E+04 | 1.04E−05 | 3.30E−010 | 3.69E+05 | 2.27E−04 | 6.15E−010 |
| BGA-5063 | — | — | — | 2.94E+04 | 2.69E−04 | 9.16E−010 |

To evaluate the binding activity of humanized antibodies to bind native B7H3 on live cells, NK92mi cells were engineered to over-express human 4Ig-B7H3. Live NK92mi/B7H3 cells were seeded in 96-well plates and were incubated with a series of dilutions of chimeric or humanized antibodies. Goat anti-human IgG was used as second antibody to detect antibody binding to the cell surface. EC₅₀ values for dose-dependent binding to human native B7H3 were determined by fitting the dose-response data to the four-parameter logistic model with GraphPad Prism. As shown in Table 21, the humanized antibodies retained binding affinity to native B7H3.

TABLE 21
Comparison on the EC₅₀ of BGA-3295 and the humanized
antibodies thereof to NK92mi/B7H3 cells by FACS

NK92mi/B7H3 binding EC50 (nM)

| Antibody | Test1 | Test2 |
|---|---|---|
| Chimeric BGA-3295 | 1.10 | — |
| BGA-4348 | 0.94 | 0.94 |
| BGA-5063 | — | 1.15 |

### Example 11. Epitope Mapping of Anti-B7H3 Antibodies

To study the binding epitope of anti-B7H3 mAb BGA-6938, domain truncated human B7H3 was generated by fusing each Ig-like domain from extracellular regions of human B7H3 (SEQ ID NO: 801), namely IgV1 (amino acids 29-139 of SEQ ID NO: 801), IgC1 (amino acids 145-238 of SEQ ID NO: 801), IgV2 (amino acids 243-357 of SEQ ID NO: 801), and IgC2 (amino acids 363-456 of SEQ ID NO: 801) with its transmembrane and intracellular domain. The truncated versions of B7H3 were fused with an N-terminal FLAG tag as well. The resulting truncated human B7H3 constructs contain an N-terminal FLAG tag followed by an Ig-like domain and B7H3 C-terminal domain including transmembrane and intracellular domains. The DNA encoding the truncated versions of B7H3 were cloned into pcDNA 3.4 vectors.

The plasmids containing these truncated B7H3 constructs were used to transfect ExpiCHO™ cells for transient protein expression, after which cells were incubated with 100 nM purified BGA-6938 as well as a reference antibody DS-7300 from Daiichi Sankyo (US 2022/0064312 A1). The binding of BGA-6938 and DS-7300 with different B7H3 truncated forms was evaluated by detection with Alexa Fluor 647 Rabbit Anti-Human IgG (Cat.: 309-605-008 Jackson ImmunoResearch). The expression of each construct was validated by the detection of FLAG tag on the N-terminus after incubating transfected cells with anti-FLAG mAb (Cat.: A01809, Genscript). BGA-6938 specifically binds to the B7H3 V1 and V2 domains and does not bind to the B7H3 Cl and C2 domains. In comparison, reference antibody DS-7300 binds to the C1 and C2 domain of B7H3 and has no binding to the V1 and V2 domains. Accordingly, BGA-6938 and DS-7300 have non-overlapping epitopes.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

It is to be understood that, if any publication is referred to herein, such reference does not constitute an admission that the publication forms a part of the common general knowledge in the art in any country.

The disclosures of all publications, patents, patent applications, and published patent applications referred to herein by an identifying citation are hereby incorporated herein by reference in their entireties.

1. An antibody drug conjugate, comprising an antibody or antigen binding fragment thereof, which is capable of binding to human B7H3; and a cytotoxic agent.

2. The antibody drug conjugate of claim 1, wherein the antibody or antigen binding fragment thereof comprises:
(i) a heavy chain variable region (VH) that comprises (a) a HCDR1 (Heavy Chain Complementarity Determining Region 1) of SEQ ID NO: 11, (b) a HCDR2 of SEQ ID NO: 2, and (c) a HCDR3 of SEQ ID NO: 14; and a light chain variable region (VL) that comprises (d) a LCDR1 (Light Chain Complementarity Determining Region 1) of SEQ ID NO: 23, (e) a LCDR2 of SEQ ID NO: 5, and (f) a LCDR3 of SEQ ID NO: 6;
(ii) a heavy chain variable region (VH) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises (d) a LCDR1 of SEQ ID NO: 4, (e) a LCDR2 of SEQ ID NO: 5, and (f) a LCDR3 of SEQ ID NO: 6;
(iii) a heavy chain variable region (VH) that comprises (a) a HCDR1 of SEQ ID NO: 11, (b) a HCDR2 of SEQ ID NO: 2, and (c) a HCDR3 of SEQ ID NO: 3; and a light chain variable region (VL) that comprises: (d) a LCDR1 of SEQ ID NO: 4, (e) a LCDR2 of SEQ ID NO: 5, and (f) a LCDR3 of SEQ ID NO: 6;
(iv) a heavy chain variable region (VH) that comprises (a) a HCDR1 of SEQ ID NO: 1, (b) a HCDR2 of SEQ ID NO: 2, and (c) a HCDR3 of SEQ ID NO: 14; and a light chain variable region (VL) that comprises (d) a LCDR1 of SEQ ID NO: 4, (e) a LCDR2 of SEQ ID NO: 5, and (f) a LCDR3 of SEQ ID NO: 6;
(v) a heavy chain variable region (VH) that comprises (a) a HCDR1 of SEQ ID NO: 1, (b) a HCDR2 of SEQ ID NO: 2, and (c) a HCDR3 of SEQ ID NO: 17; and a light chain variable region (VL) that comprises (d) a LCDR1 of SEQ ID NO: 4, (e) a LCDR2 of SEQ ID NO: 5, and (f) a LCDR3 of SEQ ID NO: 6;
(vi) a heavy chain variable region (VH) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises (d) a LCDR1 of SEQ ID NO: 20, (e) a LCDR2 of SEQ ID NO: 5, and (f) a LCDR3 of SEQ ID NO: 6;
(vii) a heavy chain variable region (VH) that comprises (a) a HCDR1 of SEQ ID NO: 1, (b) a HCDR2 of SEQ ID NO: 2, and (c) a HCDR3 of SEQ ID NO: 3; and a light chain variable region (VL) that comprises (d) a LCDR1 of SEQ ID NO: 23, (e) a LCDR2 of SEQ ID NO: 5, and (f) a LCDR3 of SEQ ID NO: 6;
(viii) a heavy chain variable region (VH) that comprises (a) a HCDR1 of SEQ ID NO: 11, (b) a HCDR2 of SEQ ID NO: 2, and (c) a HCDR3 of SEQ ID NO: 28; and a light chain variable region (VL) that comprises (d) a LCDR1 of SEQ ID NO: 23, (e) a LCDR2 of SEQ ID NO: 5, and (f) a LCDR3 of SEQ ID NO: 6; or
(ix) a heavy chain variable region (VH) that comprises (a) a HCDR1 of SEQ ID NO: 300, (b) a HCDR2 of SEQ ID NO: 1700, and (c) a HCDR3 of SEQ ID NO: 500; and a light chain variable region (VL) that comprises (d) a LCDR1 of SEQ ID NO: 600, (e) a LCDR2 of SEQ ID NO: 700, and (f) a LCDR3 of SEQ ID NO: 800.

3. The antibody drug conjugate of claim 1, having the formula: Ab-(L-(D)m)n, or a pharmaceutically acceptable salt of solvate thereof, wherein:
Ab is the antibody or antigen binding fragment thereof;
L is a linker;
D is a residue of the cytotoxic agent;
m is an integer from 1 to 8; and
n is from 1 to 10.

4. The antibody drug conjugate of claim 3, wherein m is 1.

5. The antibody drug conjugate of claim 3, wherein n is from 3 to 10.

6. The antibody drug conjugate of claim 5, wherein n is about 8.

7. The antibody drug conjugate of claim 3, having Formula (II): 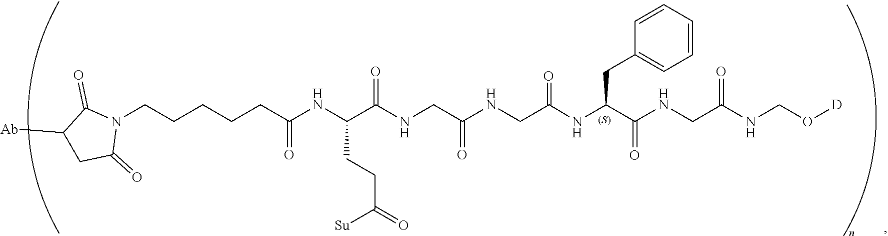
or a tautomer, stereoisomer, or pharmaceutically acceptable salt thereof, wherein Su is a hydrophilic residue.

8. The antibody drug conjugate of claim 7, wherein Su is

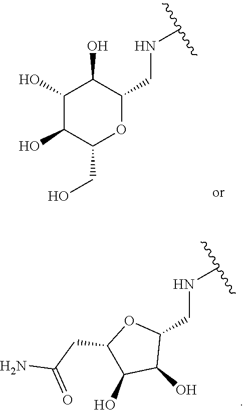 [SMILES: *NC[C@@H]1O[C@H](CO)[C@@H](O)[C@H](O)[C@H]1O ; *NC[C@H]1O[C@@H](CC(N)=O)[C@H](O)[C@@H]1O]

9. The antibody drug conjugate of claim 3, having Formula (III): 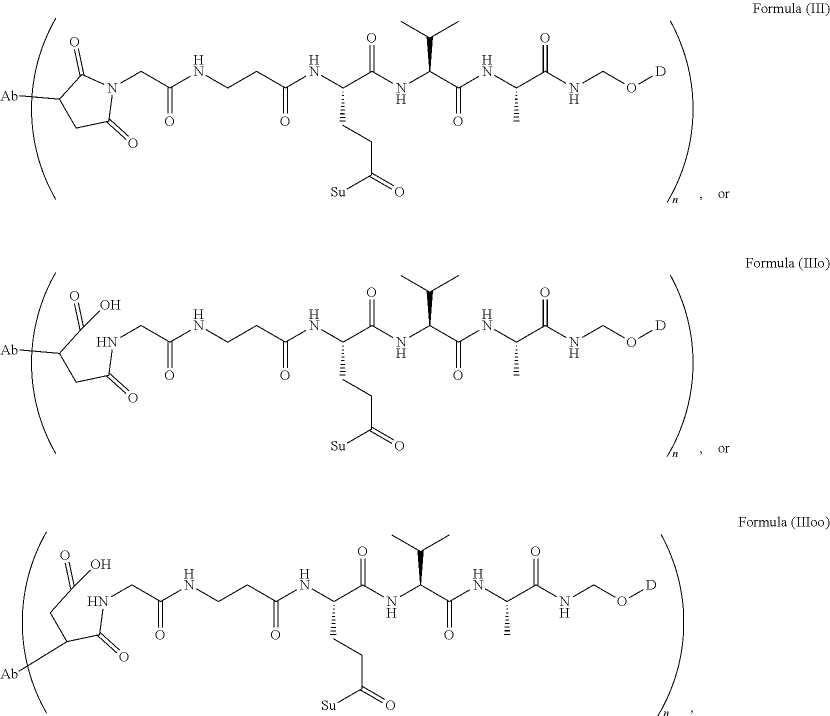
or a tautomer, stereoisomer, or pharmaceutically acceptable salt thereof, wherein Su is a hydrophilic residue.

10. The antibody drug conjugate of claim 9, wherein Su is

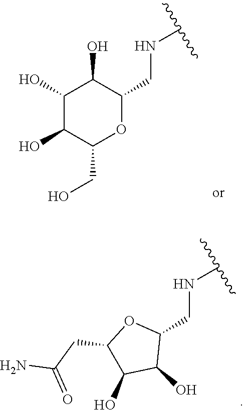 [SMILES: *NC[C@@H]1O[C@H](CO)[C@@H](O)[C@H](O)[C@H]1O ; *NC[C@H]1O[C@@H](CC(N)=O)[C@H](O)[C@@H]1O]

11. The antibody drug conjugate of claim 3, wherein D is: 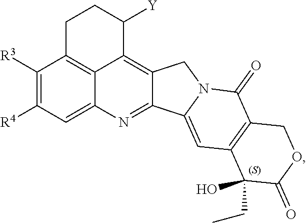
wherein:
Y is -A-B—C′-D′-*, wherein * marks the bond where D connects to the antibody-drug conjugate;
A is a bond, CR¹R² or N—R¹;
B is a bond, —C(═O)—, or —C(═O)O—;
C′ is a bond, or a divalent group, wherein the divalent group is unsubstituted or substituted C₁₋₈ alkyl, unsubstituted or substituted cycloalkyl, unsubstituted or substituted heterocyclyl, unsubstituted or substituted aryl, or unsubstituted or substituted heteroaryl;
D′ is a bond, NH, or 0;
each of R¹ and R² is, independently, hydrogen, halogen, substituted or unsubstituted alkyl, or substituted or unsubstituted alkoxyl; or R¹ and R² together with the atom to which they are attached form unsubstituted or substituted cycloalkyl, unsubstituted or substituted heterocyclyl, unsubstituted or substituted aryl, or unsubstituted or substituted heteroaryl; and
each of R¹ and R⁴ is, independently, hydrogen, halogen, substituted or unsubstituted alkyl, or substituted or unsubstituted alkoxyl; or R³ and R⁴ together with the atoms to which they are attached form unsubstituted or substituted cycloalkyl, unsubstituted or substituted heterocyclyl, unsubstituted or substituted aryl, or unsubstituted or substituted heteroaryl.

12. The antibody drug conjugate of claim 3, wherein D is: 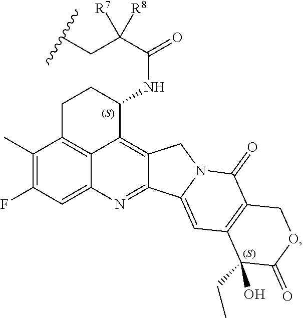
wherein R⁷ and R⁸ are each independently hydrogen, halogen, or alkyl.

13. The antibody drug conjugate of claim 3, wherein D is:

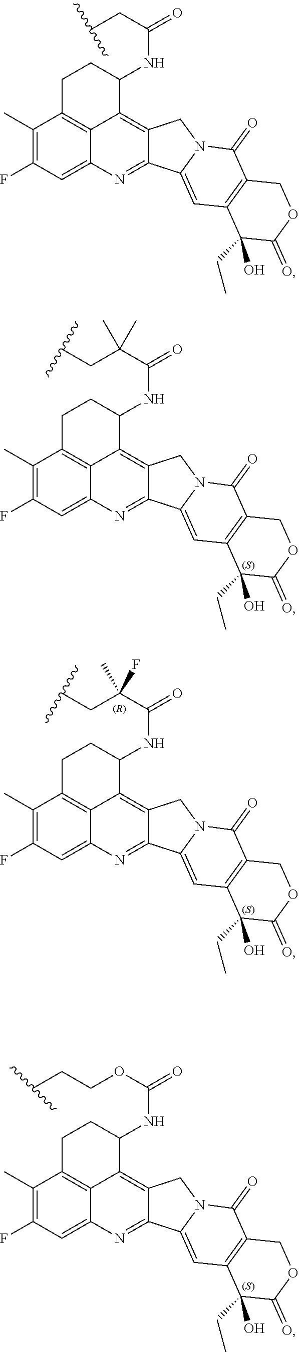

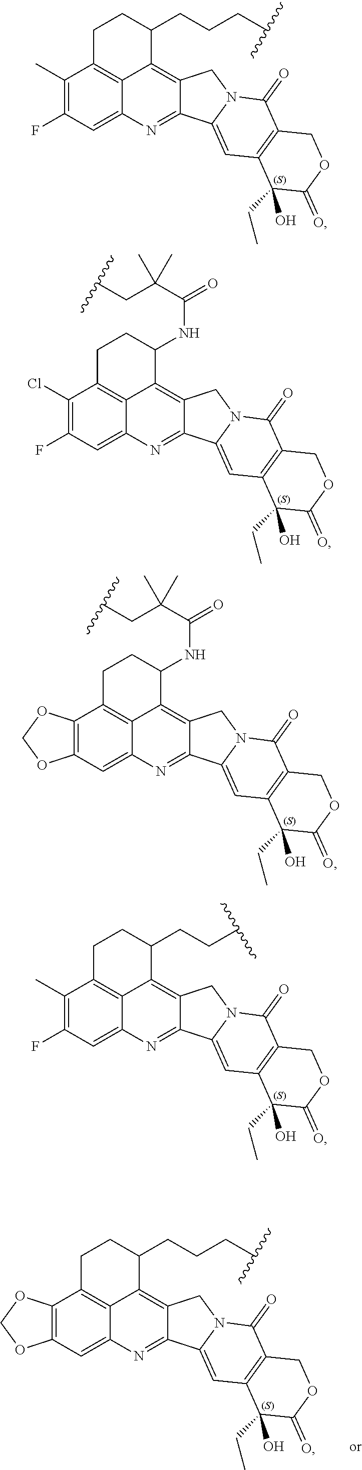

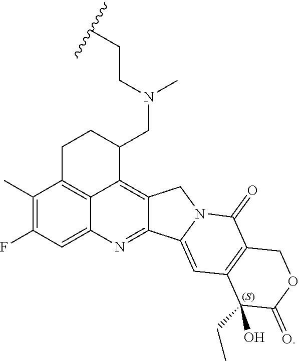

14. The antibody drug conjugate of claim 13, wherein D is:

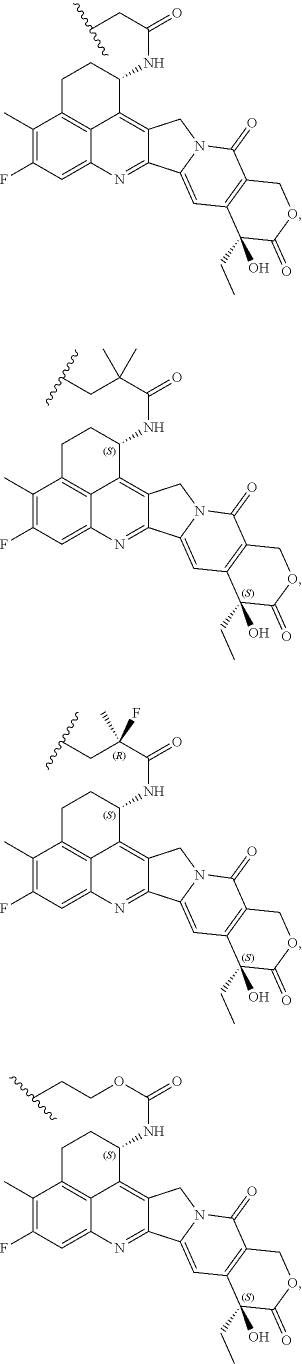

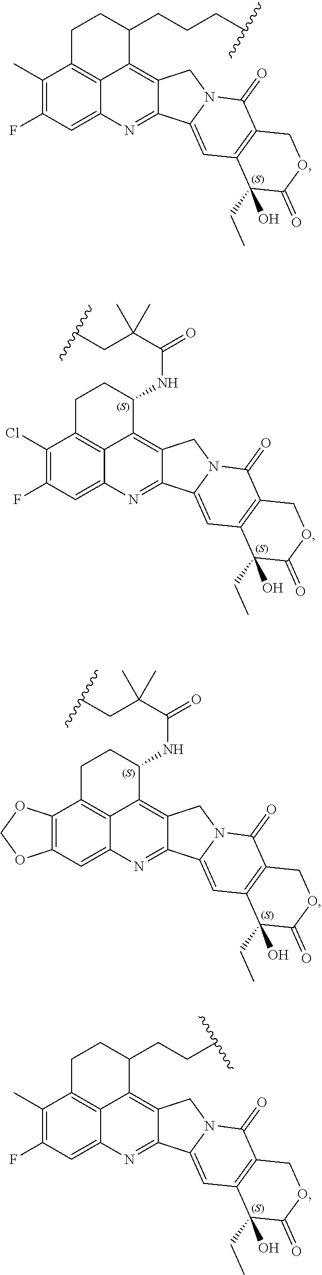

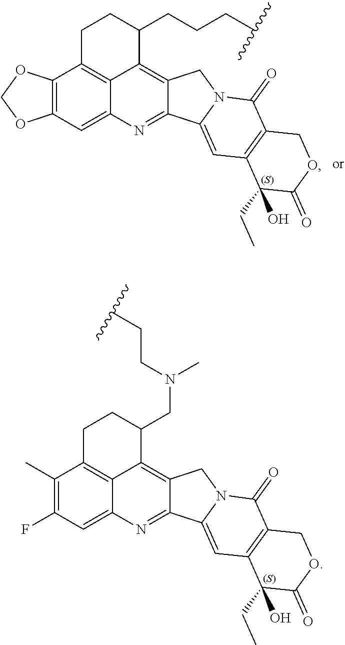

15. The antibody drug conjugate of claim 3, having one of the following formulas, or a tautomer, stereoisomer, or pharmaceutically acceptable salt thereof:

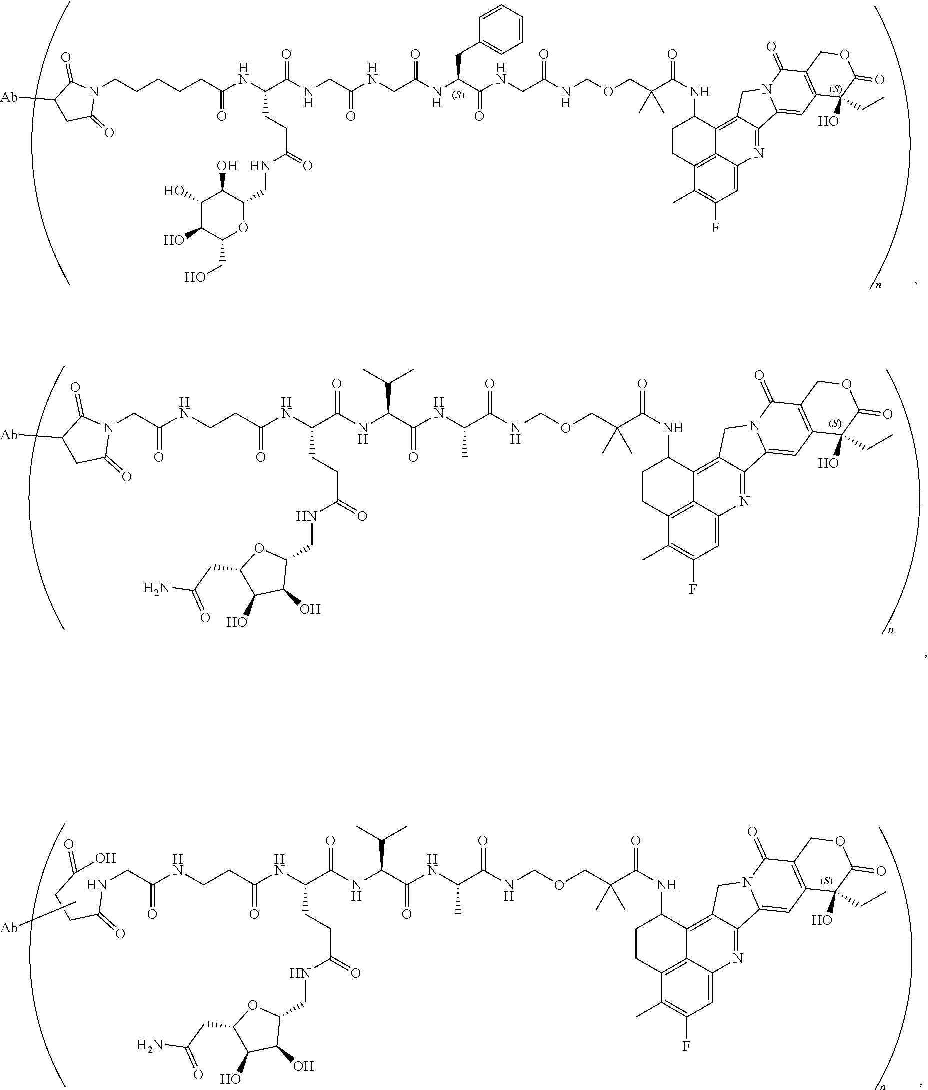

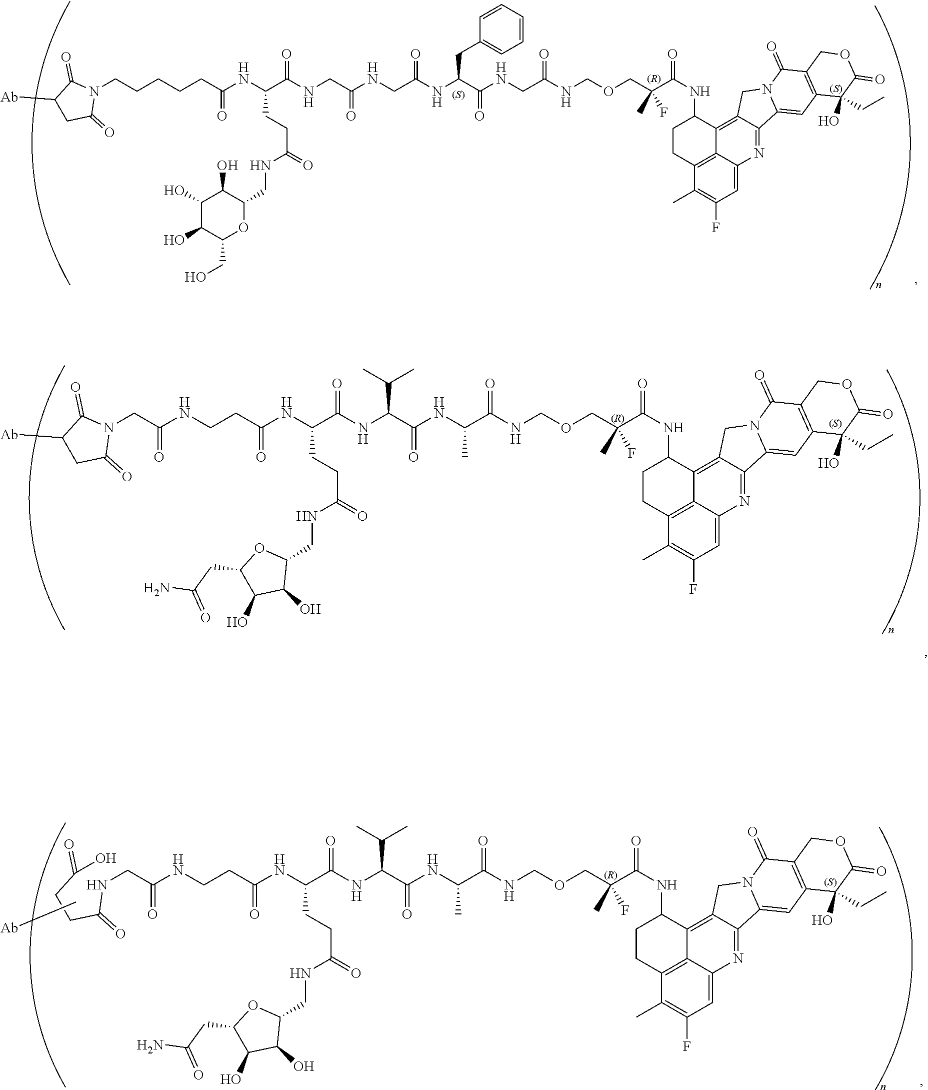

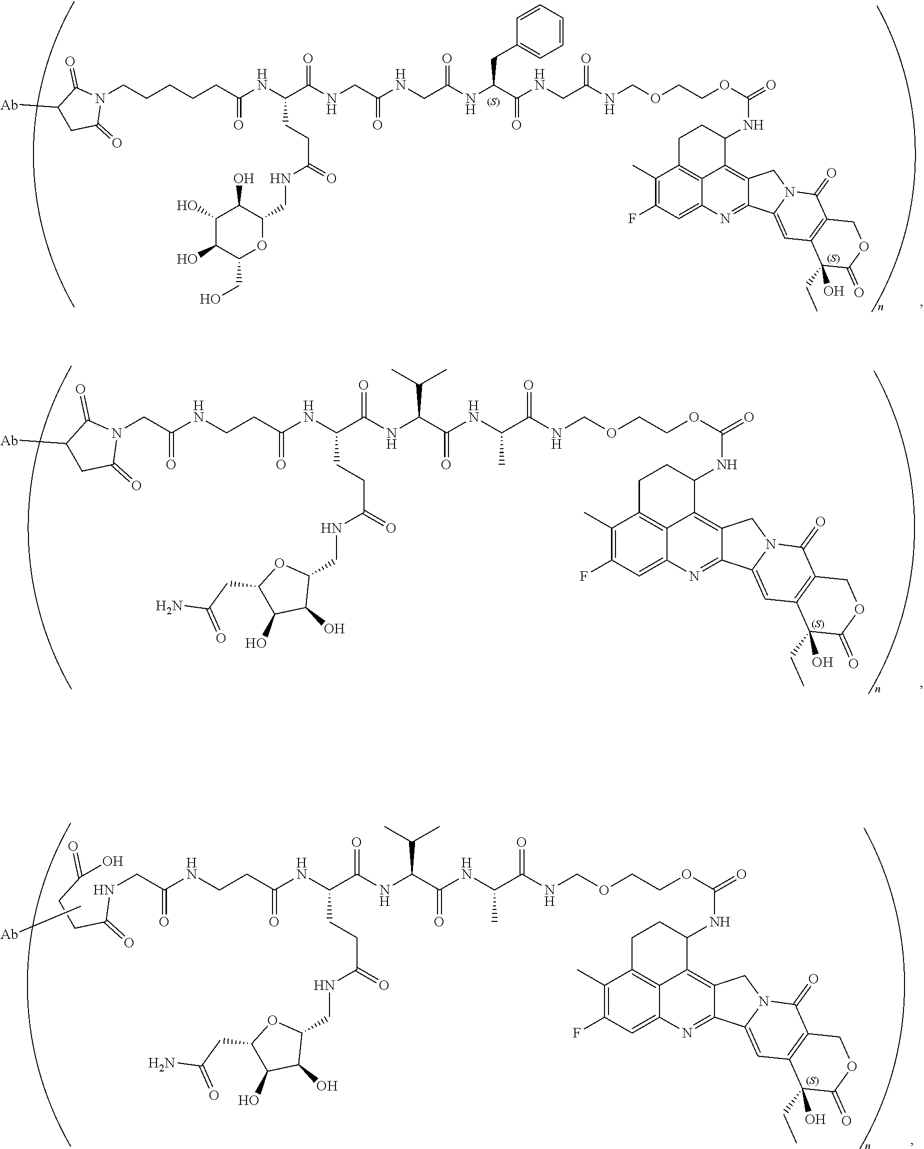

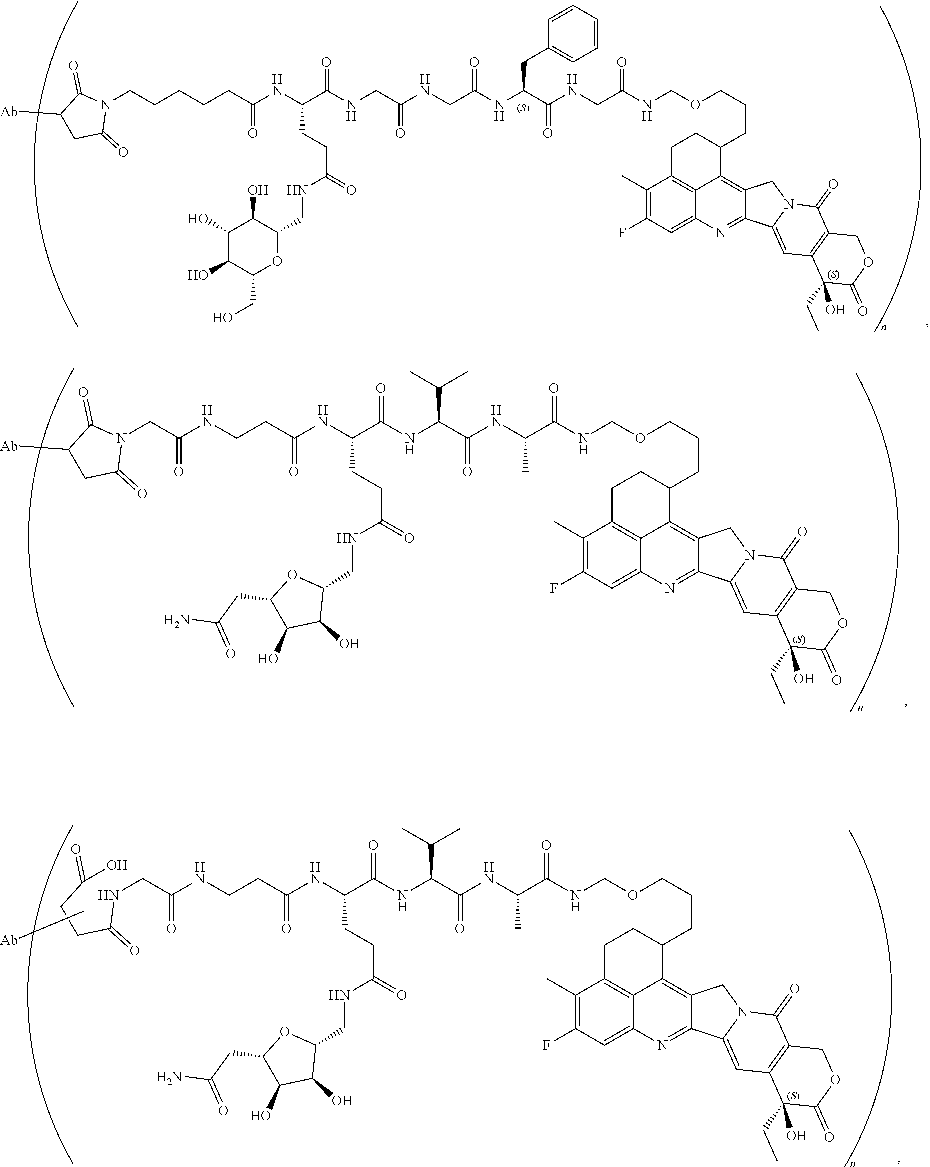

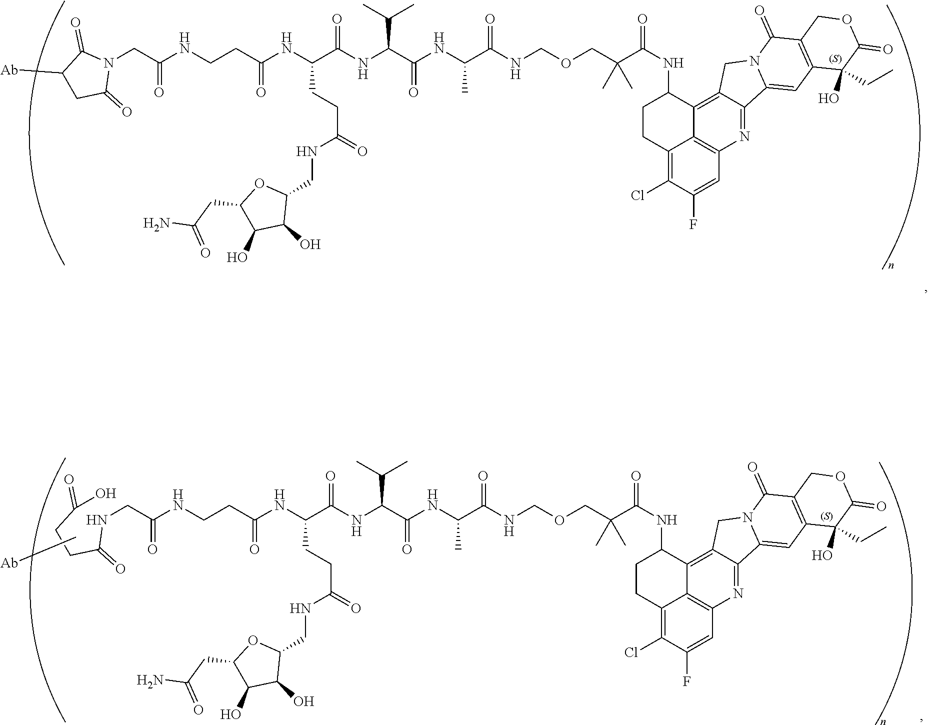

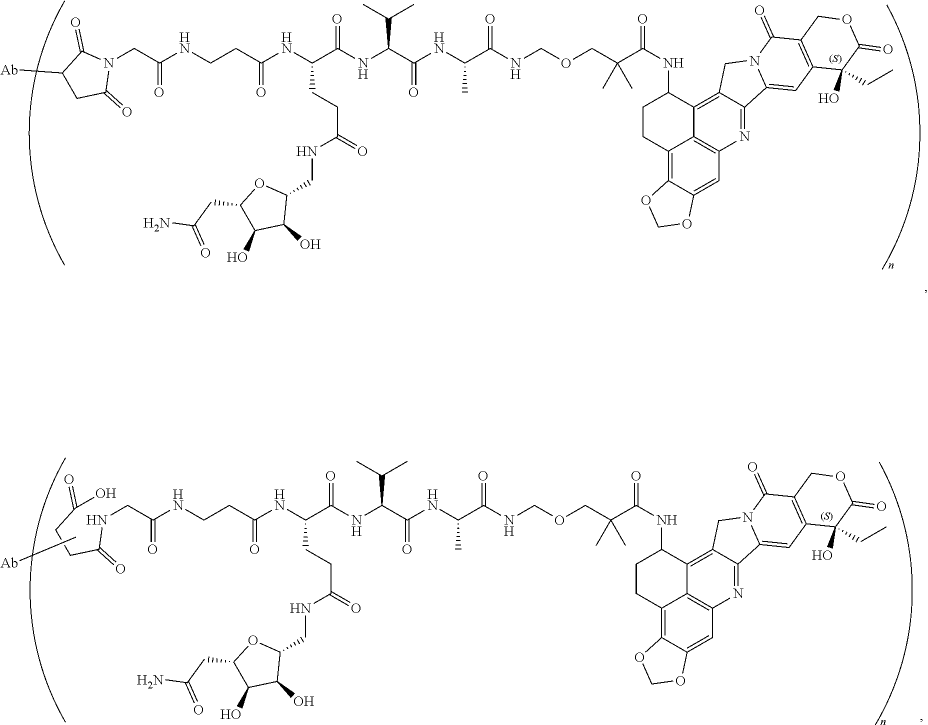

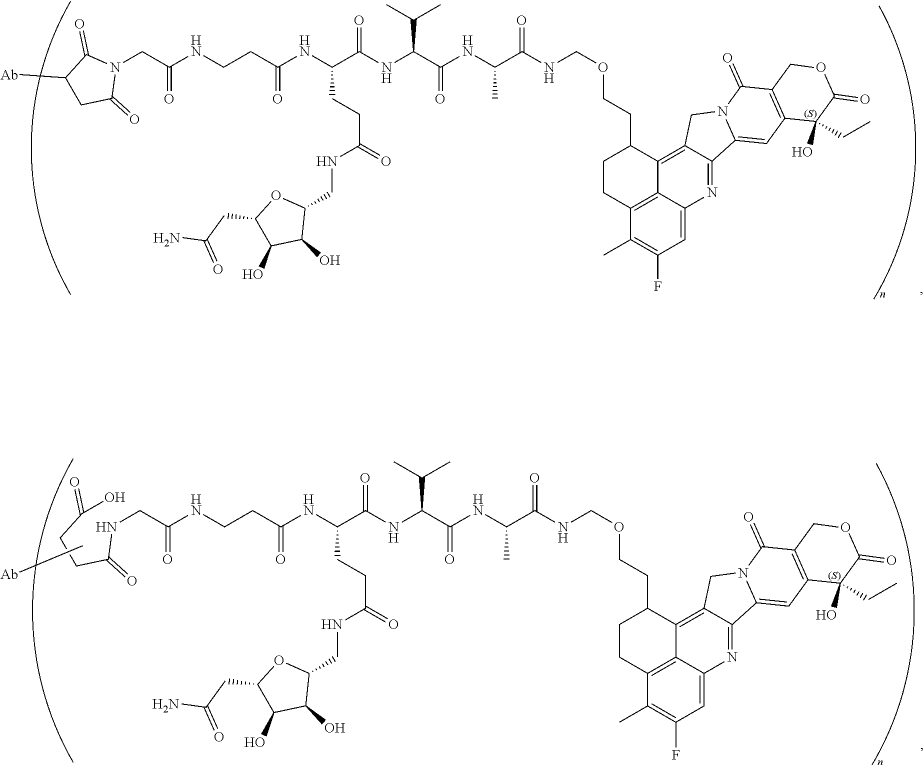

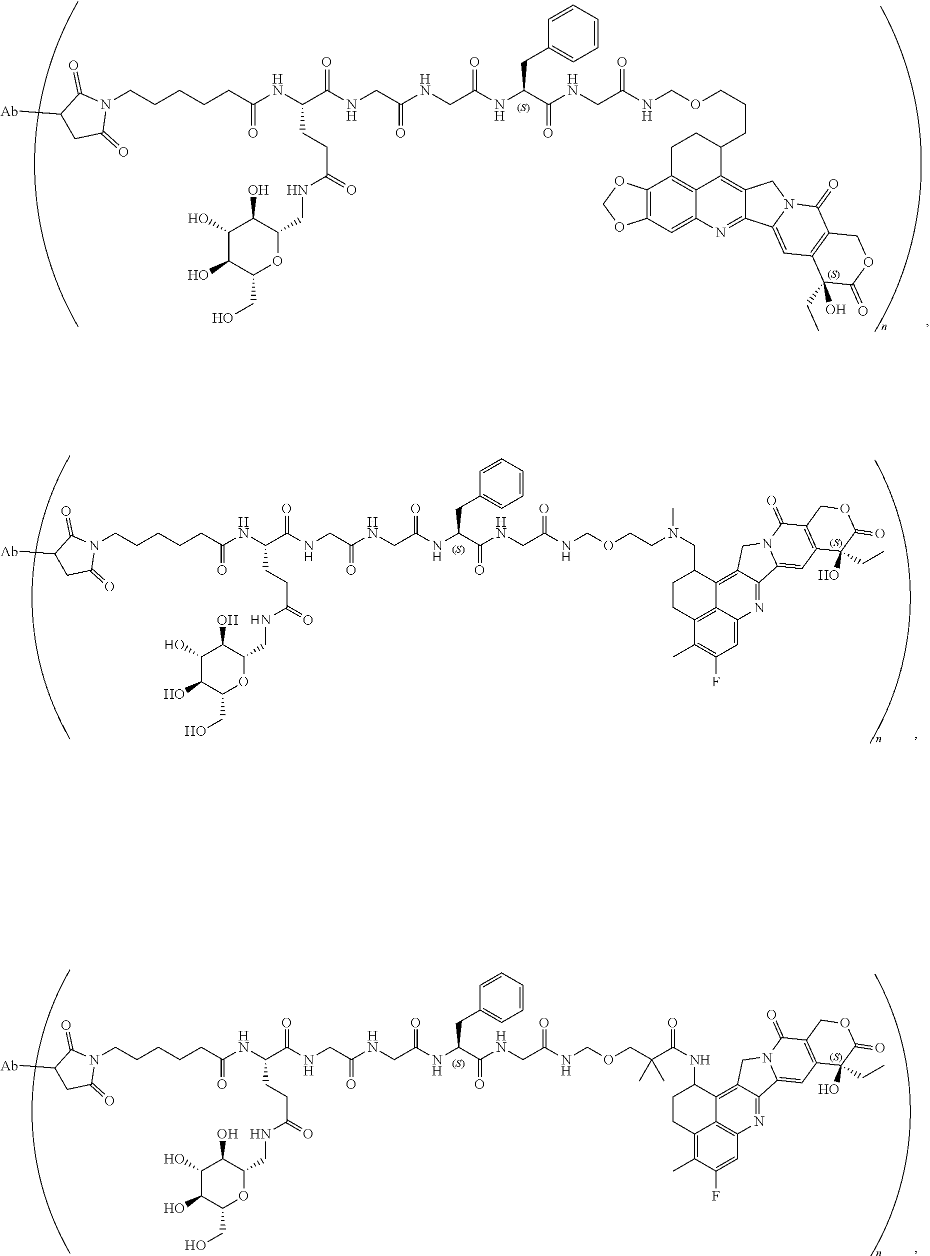

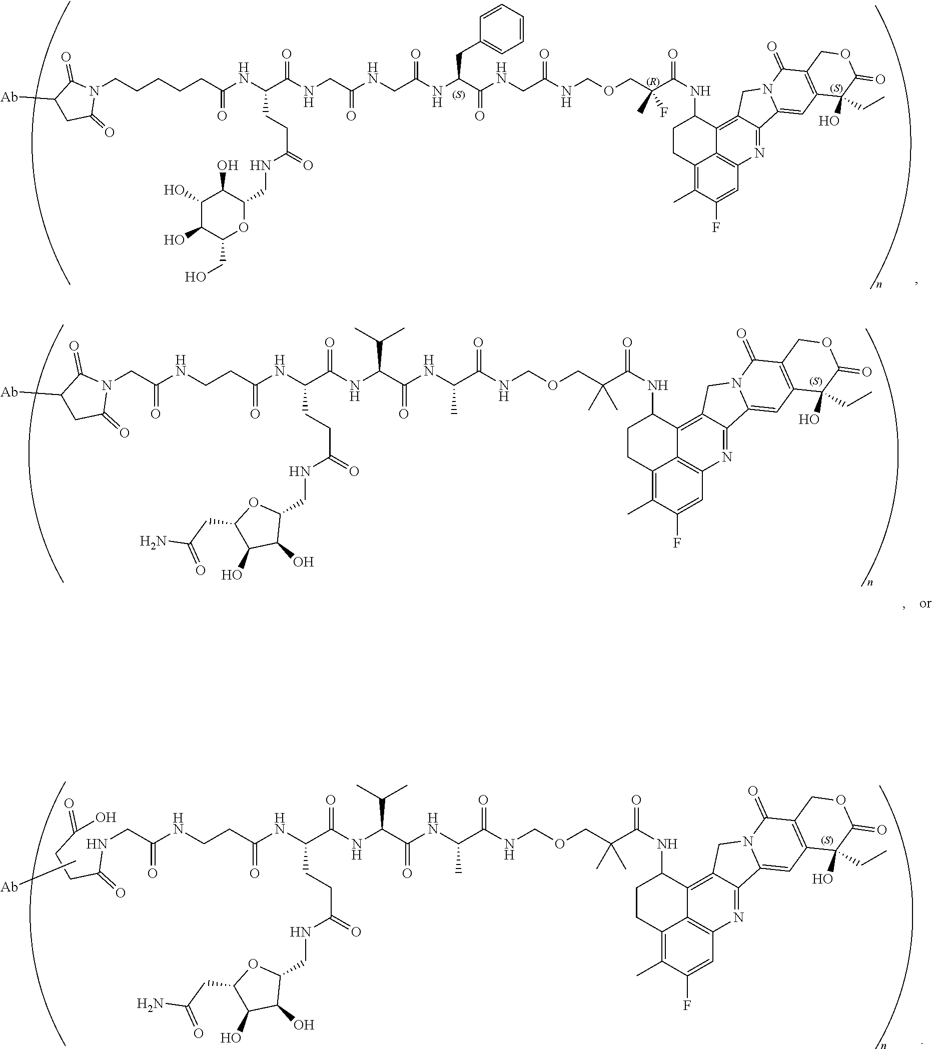

16. The antibody drug conjugate of claim 3, having one of the following, formulas, or a tautomer, stereoisomer, or pharmaceutically acceptable salt thereof:

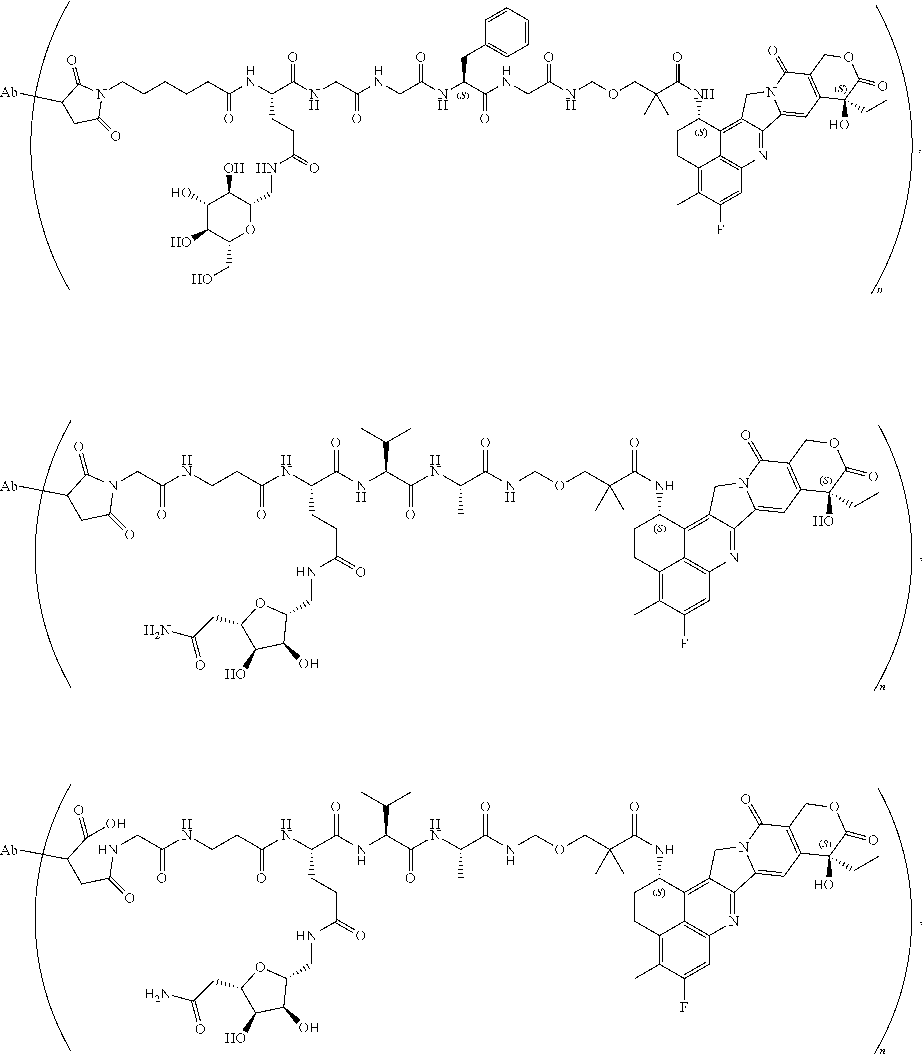

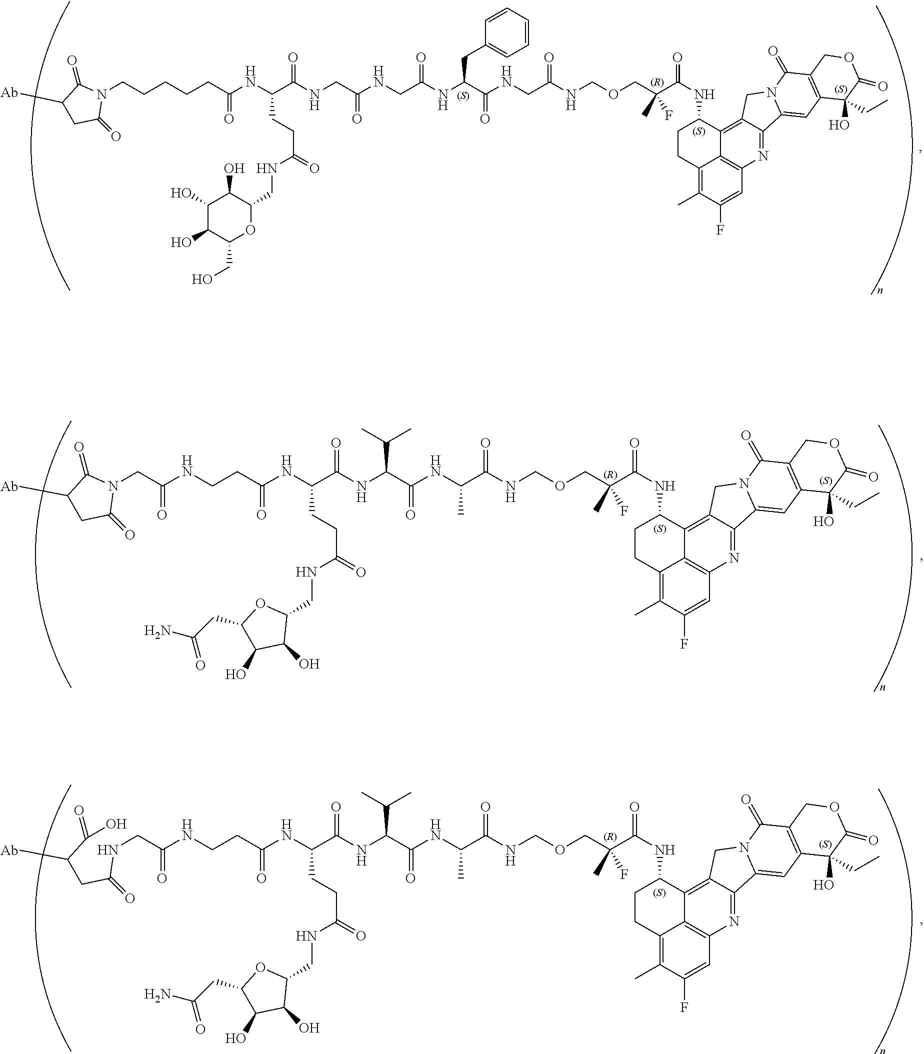

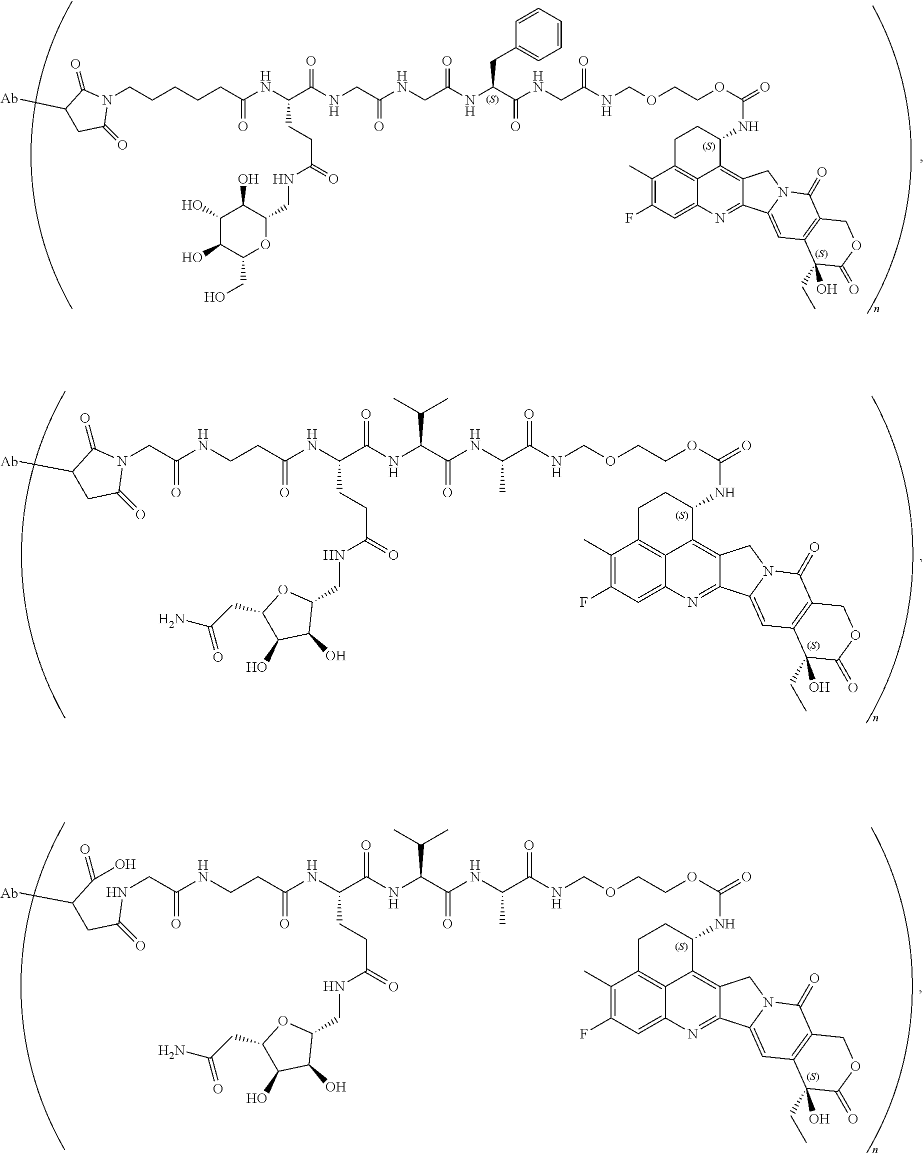

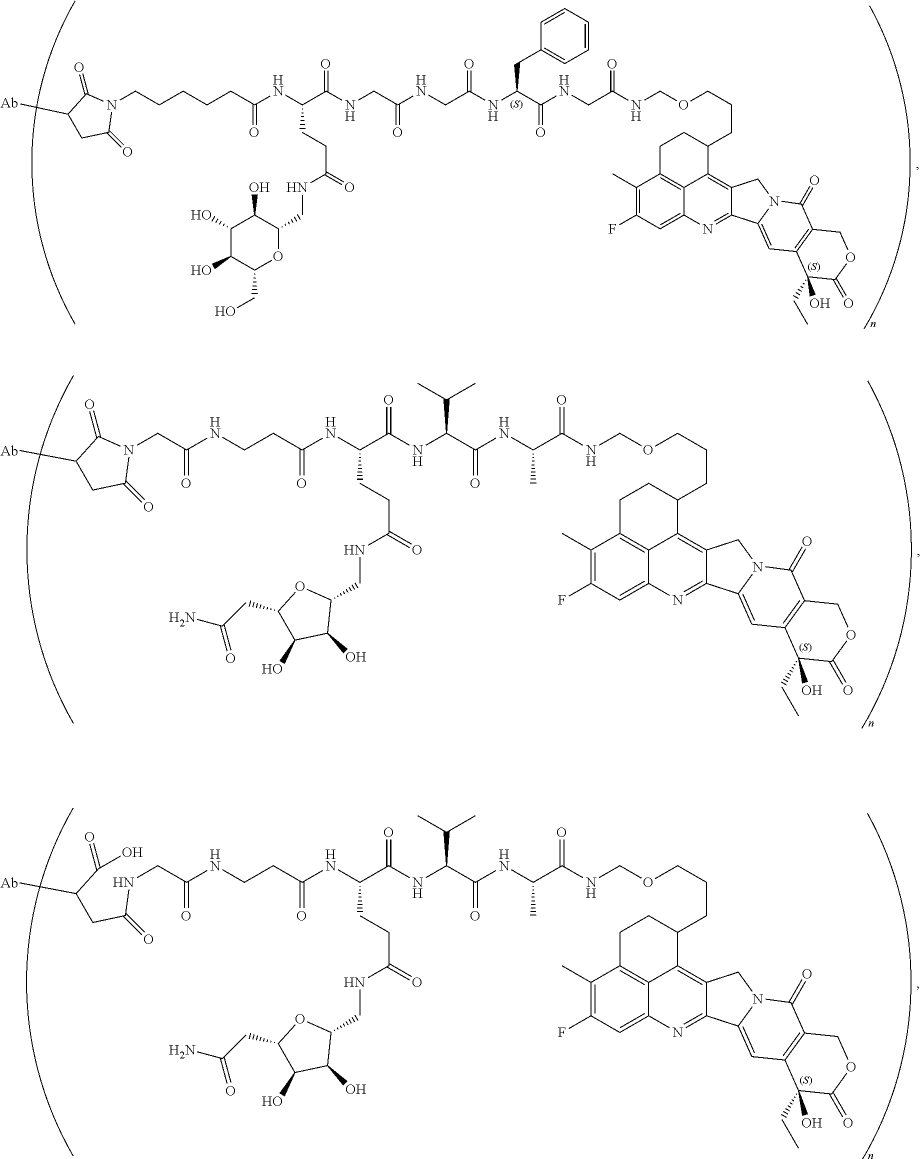

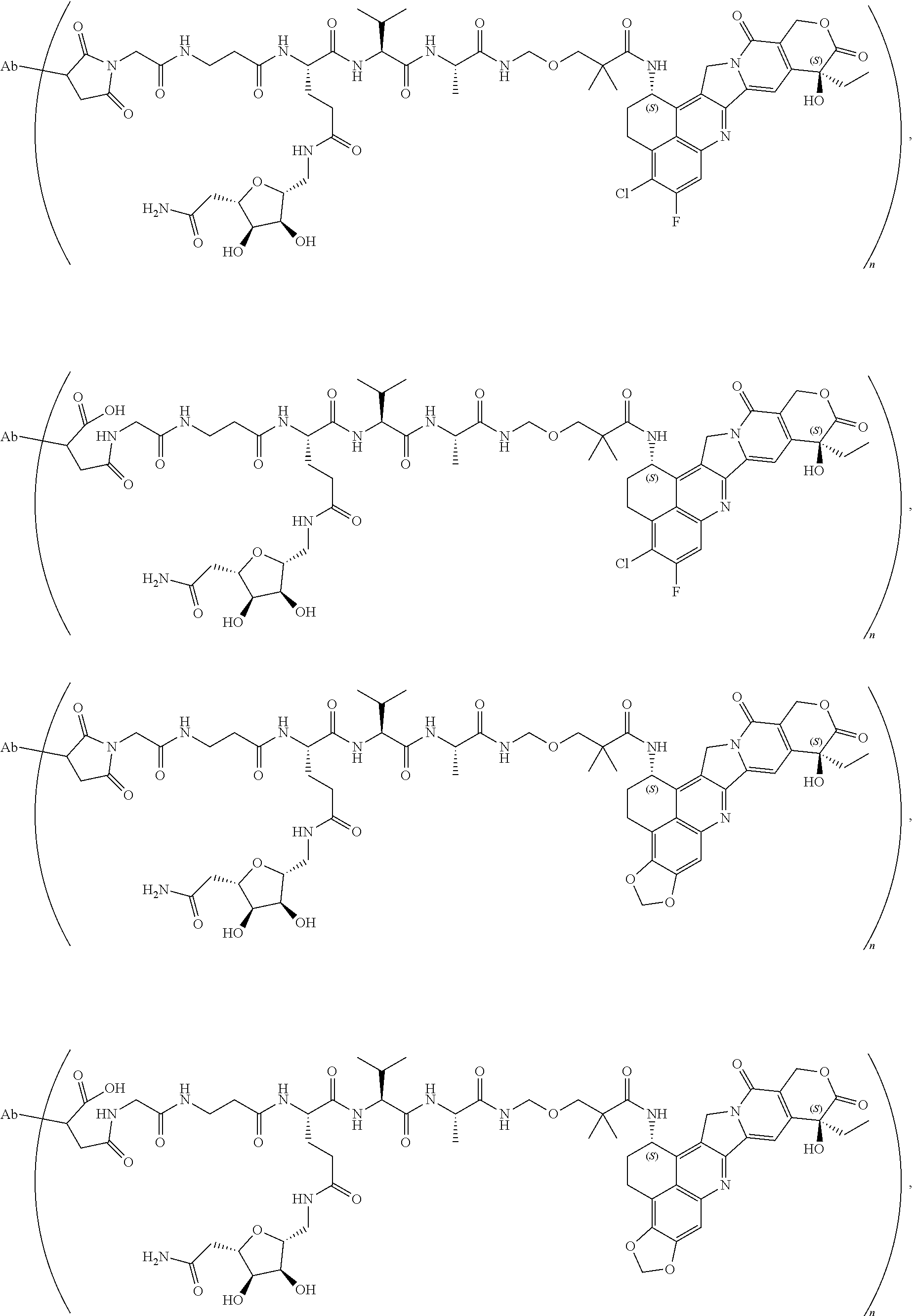

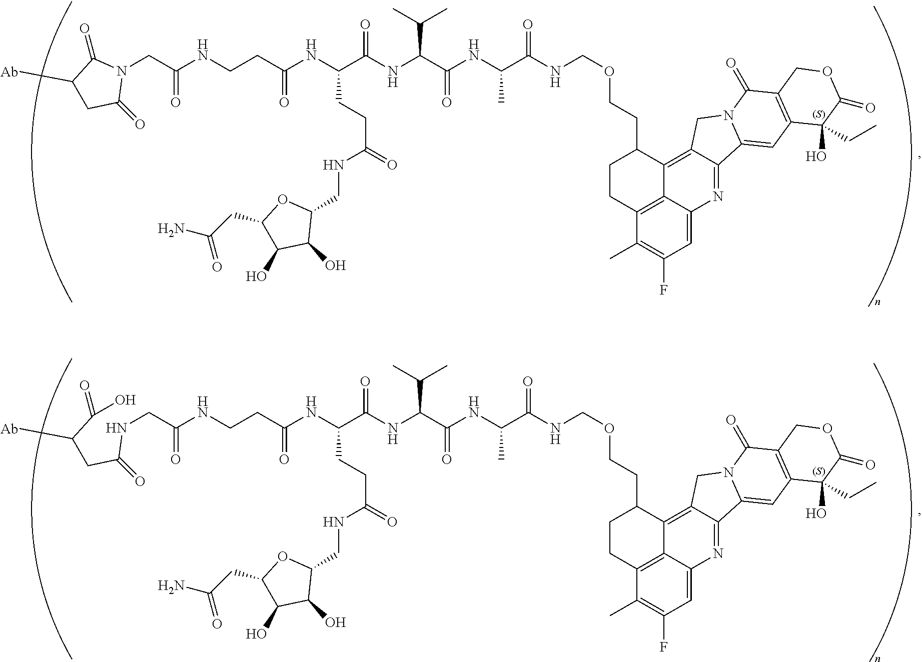

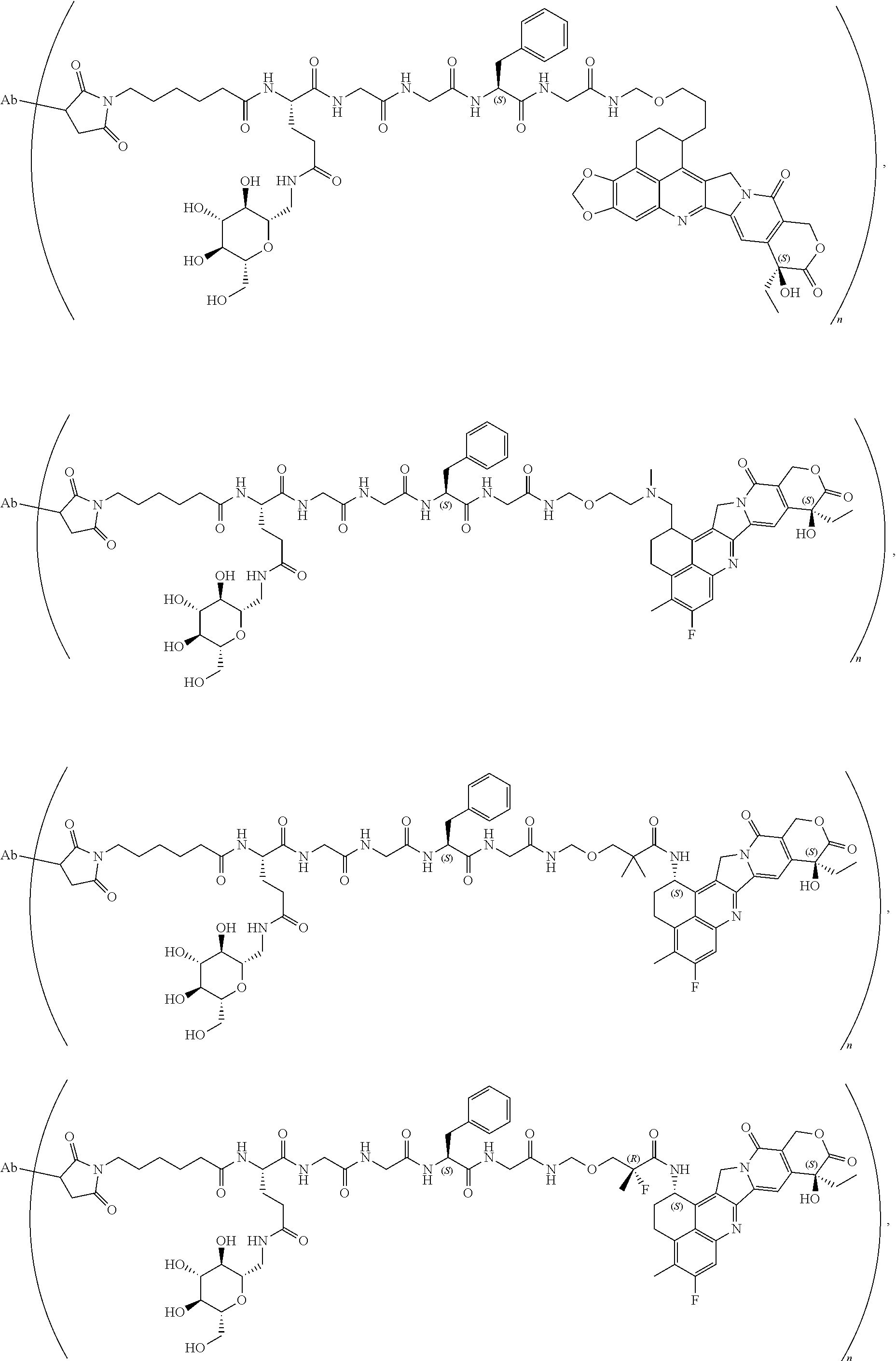

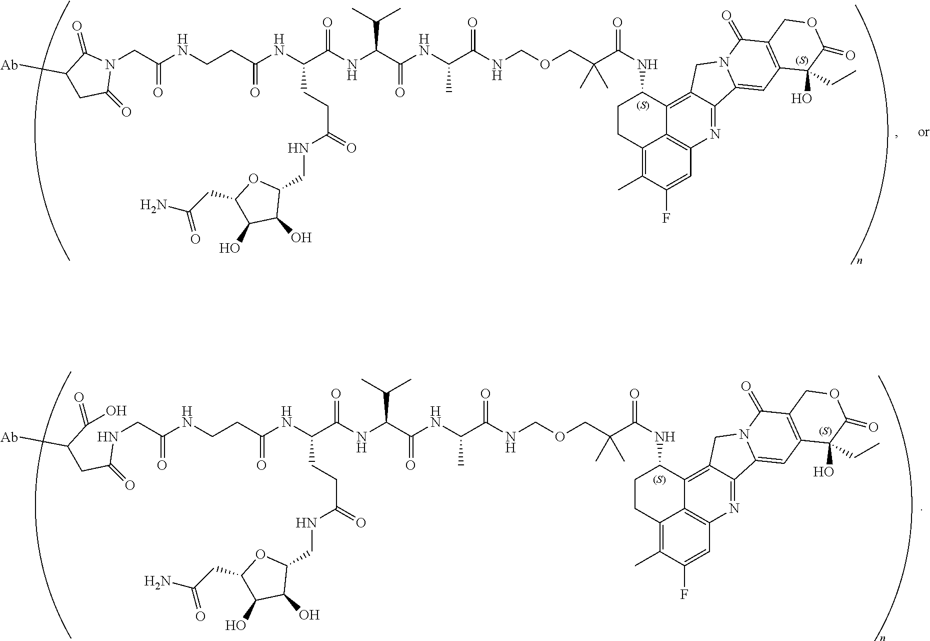

17. The antibody drug conjugate of claim 1, wherein the antibody or antigen-binding fragment comprises:
(i) a heavy chain variable region comprising an amino acid sequence at least 90, 91, 92, 93, 94, 95, 96, 97, 98 or 99% identical to SEQ ID NO: 26, and a light chain variable region comprising an amino acid sequence at least 90, 91, 92, 93, 94, 95, 96, 97, 98 or 99% identical to SEQ ID NO: 24;
(ii) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 8;
(iii) a heavy chain variable region comprising an amino acid sequence at least 90, 91, 92, 93, 94, 95, 96, 97, 98 or 99% identical to SEQ ID NO: 12, and a light chain variable region comprising an amino acid sequence at least 90, 91, 92, 93, 94, 95, 96, 97, 98 or 99% identical to SEQ ID NO: 8;
(iv) a heavy chain variable region comprising an amino acid sequence at least 90, 91, 92, 93, 94, 95, 96, 97, 98 or 99% identical to SEQ ID NO: 15, and a light chain variable region comprising an amino acid sequence at least 90, 91, 92, 93, 94, 95, 96, 97, 98 or 99% identical to SEQ ID NO: 8;
(v) a heavy chain variable region comprising an amino acid sequence at least 90, 91, 92, 93, 94, 95, 96, 97, 98 or 99% identical to SEQ ID NO: 18, and a light chain variable region comprising an amino acid sequence at least 90, 91, 92, 93, 94, 95, 96, 97, 98 or 99% identical to SEQ ID NO: 8;
(vi) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 21;
(vii) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 24;
(viii) a heavy chain variable region comprising an amino acid sequence at least 90, 91, 92, 93, 94, 95, 96, 97, 98 or 99% identical to SEQ ID NO: 29, and a light chain variable region comprising an amino acid sequence at least 90, 91, 92, 93, 94, 95, 96, 97, 98 or 99% identical to SEQ ID NO: 24; or
(ix) a heavy chain variable region comprising an amino acid sequence at least 90, 91, 92, 93, 94, 95, 96, 97, 98 or 99% identical to SEQ ID NO: 1800, and a light chain variable region comprising an amino acid sequence at least 90, 91, 92, 93, 94, 95, 96, 97, 98 or 99% identical to SEQ ID NO: 1400.

18. The antibody drug conjugate of claim 17, wherein one, two, three, four, five, six, seven, eight, nine, or ten amino acids within each of SEQ ID NOs: 26 and 24, each of SEQ ID NOs: 7 and 8, each of SEQ ID NOs: 12 and 8, each of SEQ ID NOs: 15 and 8, each of SEQ ID NOs: 18 and 8, each of SEQ ID NOs: 7 and 21, each of SEQ ID Nos: 7 and 24, each of SEQ ID Nos: 29 and 24, or each of SEQ ID NOs: 1800 and 1400, have been inserted, deleted or substituted in the antibody or antigen-binding fragment.

19. The antibody drug conjugate of claim 1, wherein the antibody or antigen-binding fragment comprises:
(i) a heavy chain variable region comprising SEQ ID NO: 26, and a light chain variable region comprising SEQ ID NO: 24;
(ii) a heavy chain variable region comprising SEQ ID NO: 7, and a light chain variable region comprising SEQ ID NO: 8;
(iii) a heavy chain variable region comprising SEQ ID NO: 12, and a light chain variable region comprising SEQ ID NO: 8;
(iv) a heavy chain variable region comprising SEQ ID NO: 15, and a light chain variable region comprising SEQ ID NO: 8;
(v) a heavy chain variable region comprising SEQ ID NO: 18, and a light chain variable region comprising SEQ ID NO: 8;
(vi) a heavy chain variable region comprising SEQ ID NO: 7, and a light chain variable region comprising SEQ ID NO: 21;
(vii) a heavy chain variable region comprising SEQ ID NO: 7, and a light chain variable region comprising SEQ ID NO: 24;
(viii) a heavy chain variable region comprising SEQ ID NO: 29, and a light chain variable region comprising SEQ ID NO: 24; or
(ix) a heavy chain variable region comprising SEQ ID NO: 1800, and a light chain variable region comprising SEQ ID NO: 1400.

20. (canceled)

21. The antibody drug conjugate of claim 1, wherein the antibody or antigen-binding fragment comprises a scFv comprising a VII having the amino acid of SEQ ID NO: 26 and a VL having an amino acid of SEQ ID NO: 24, optionally the VH and VL are connected via an amino acid linker, optionally the amino acid linker is any sequence of SEQ ID NO: 35 to SEQ ID NO: 77.

22. The antibody drug conjugate of claim 21, wherein the antibody or antigen-binding fragment comprises a scFv having the amino acid sequence of SEQ ID NO: 32.

23.-24. (canceled)

25. An antibody drug conjugate of claim 1, wherein the antibody or antigen binding fragment thereof specifically binds to an epitope comprising or consisting of amino acid residues 29-139 of human 4Ig-B7H3 (SEQ ID NO: 801); and/or amino acid residues 243-357 of human 4Ig-B7H3 (SEQ ID NO: 801); and/or amino acid residues 145-238 of human 4Ig-B7H3 (SEQ ID NO: 801); and/or amino acid residues 363-456 of human 4Ig-B7H3 (SEQ ID NO: 801).

26. A pharmaceutical composition comprising the antibody drug conjugate of claim 1 and a pharmaceutically acceptable carrier.

27. A method of treating a 4Ig-B7H3 positive cancer, comprising administering to a patient in need thereof an effective amount of the antibody drug conjugate of claim 1, or a pharmaceutical composition comprising the antibody drug conjugate of claim 1 and a pharmaceutically acceptable carrier.

28.-36. (canceled)

37. A compound of Formula (IIIa): 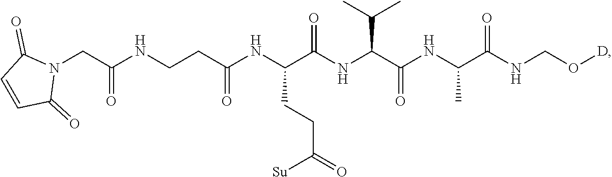
or a pharmaceutically acceptable salt thereof, wherein Su is 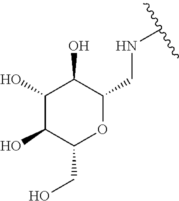 [SMILES: *NC[C@@H]1O[C@H](CO)[C@@H](O)[C@H](O)[C@H]1O]
and D is a residue of a cytotoxic agent.

38.-39. (canceled)


SEQUENCE LISTING (1900 sequences)

SEQ ID NO: 1 - protein, 6 aa, synthetic construct
  REGION 1..6: note = Synthetic: HCDR1 (Kabat)
TSNWWN

SEQ ID NO: 2 - protein, 16 aa, synthetic construct
  REGION 1..16: note = Synthetic: HCDR2 (Kabat)
EIYPTGNTNYSPSLKS

SEQ ID NO: 3 - protein, 7 aa, synthetic construct
  REGION 1..7: note = Synthetic: HCDR3 (Kabat)
GWNWFDP

SEQ ID NO: 4 - protein, 12 aa, synthetic construct
  REGION 1..12: note = Synthetic: LCDR1 (Kabat)
RTSQSINGILLA

SEQ ID NO: 5 - protein, 7 aa, synthetic construct
  REGION 1..7: note = Synthetic: LCDR2 (Kabat)
GASSRAT

SEQ ID NO: 6 - protein, 9 aa, synthetic construct
  REGION 1..9: note = Synthetic: LCDR3 (Kabat)
QQYGSSPRT

SEQ ID NO: 7 - protein, 116 aa, synthetic construct
  REGION 1..116: note = Synthetic: VH AA
QVQLQESGPGLVKPSGTLSLTCAVSGGSISTSNWWNWVRQSPGKGLEWIGEIYPTGNTNYSPSLKSRVTISIDKSKNQFSLKLNSVTAADTAVYYCARGWNWFDPWGQGTLVTVSS

SEQ ID NO: 8 - protein, 108 aa, synthetic construct
  REGION 1..108: note = Synthetic: VL AA
EIVLTQSPGTLSLSPGERATLSCRTSQSINGILLAWYQQKPGQAPRLLIYGASSRATGIPDRFSGSGSGTDFTLSISRLEPEDFAVYYCQQYGSSPRTFGQGTKVEIK

SEQ ID NO: 9 - DNA, 348 bp, synthetic construct
  misc_feature 1..348: note = Synthetic: VH DNA
  source 1..348: mol_type = other DNA
caggtgcagctgcaggagtcgggcccaggactggtgaagccttcggggaccctgtccctcacctgcgctgtctctggtggctccatcagtactagtaactggtggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatggaactggttcgacccctggggccagggaaccctggtcaccgtctcctca

SEQ ID NO: 10 - DNA, 324 bp, synthetic construct
  misc_feature 1..324: note = Synthetic: VL DNA
  source 1..324: mol_type = other DNA
gaaattgtgttgacgcagtctccaggcaccctgtctttgtctccaggggaaagagccaccctctcctgcaggaccagtcagagtattaacggcatcttgttagcctggtaccagcagaaacctggccaggctcccaggctcctcatctatggtgcatccagcagggccactggcatccccgacaggttcagtggcagtgggtctgggacagacttcactctcagcatcagcagactggagcctgaagattttgcagtgtattactgtcagcagtatggcagttcacctcggacgttcggccaagggaccaaggtggaaatcaaa

SEQ ID NO: 11 - protein, 6 aa, synthetic construct
  REGION 1..6: note = Synthetic: HCDR1 (Kabat)
TSNYWN

SEQ ID NO: 12 - protein, 116 aa, synthetic construct
  REGION 1..116: note = Synthetic: VH AA
QVQLQESGPGLVKPSGTLSLTCAVSGGSISTSNYWNWVRQSPGKGLEWIGEIYPTGNTNYSPSLKSRVTISIDKSKNQFSLKLNSVTAADTAVYYCARGWNWFDPWGQGTLVTVSS

SEQ ID NO: 13 - DNA, 348 bp, synthetic construct
  misc_feature 1..348: note = Synthetic: VH DNA
  source 1..348: mol_type = other DNA
caggtgcagctgcaggagtcgggcccaggactggtgaagccttcggggaccctgtccctcacctgcgctgtctctggtggctccatcagtactagtaactactggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatggaactggttcgacccctggggccagggaaccctggtcaccgtctcctca

SEQ ID NO: 14 - protein, 7 aa, synthetic construct
  REGION 1..7: note = Synthetic: HCDR3 (Kabat)
GYNWFDP

SEQ ID NO: 15 - protein, 116 aa, synthetic construct
  REGION 1..116: note = Synthetic: VH AA
QVQLQESGPGLVKPSGTLSLTCAVSGGSISTSNWWNWVRQSPGKGLEWIGEIYPTGNTNYSPSLKSRVTISIDKSKNQFSLKLNSVTAADTAVYYCARGYNWFDPWGQGTLVTVSS

SEQ ID NO: 16 - DNA, 348 bp, synthetic construct
  misc_feature 1..348: note = Synthetic: VH DNA
  source 1..348: mol_type = other DNA
caggtgcagctgcaggagtcgggcccaggactggtgaagccttcggggaccctgtccctcacctgcgctgtctctggtggctccatcagtactagtaactggtggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatacaactggttcgacccctggggccagggaaccctggtcaccgtctcctca

SEQ ID NO: 17 - protein, 7 aa, synthetic construct
  REGION 1..7: note = Synthetic: HCDR3 (Kabat)
GWNYFDP

SEQ ID NO: 18 - protein, 116 aa, synthetic construct
  REGION 1..116: note = Synthetic: VH AA
QVQLQESGPGLVKPSGTLSLTCAVSGGSISTSNWWNWVRQSPGKGLEWIGEIYPTGNTNYSPSLKSRVTISIDKSKNQFSLKLNSVTAADTAVYYCARGWNYFDPWGQGTLVTVSS

SEQ ID NO: 19 - DNA, 348 bp, synthetic construct
  misc_feature 1..348: note = Synthetic: VH DNA
  source 1..348: mol_type = other DNA
caggtgcagctgcaggagtcgggcccaggactggtgaagccttcggggaccctgtccctcacctgcgctgtctctggtggctccatcagtactagtaactggtggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatggaactacttcgacccctggggccagggaaccctggtcaccgtctcctca

SEQ ID NO: 20 - protein, 12 aa, synthetic construct
  REGION 1..12: note = Synthetic: LCDR1 (Kabat)
RTSQSIQGILLA

SEQ ID NO: 21 - protein, 108 aa, synthetic construct
  REGION 1..108: note = Synthetic: VL AA
EIVLTQSPGTLSLSPGERATLSCRTSQSIQGILLAWYQQKPGQAPRLLIYGASSRATGIPDRFSGSGSGTDFTLSISRLEPEDFAVYYCQQYGSSPRTFGQGTKVEIK

SEQ ID NO: 22 - DNA, 324 bp, synthetic construct
  misc_feature 1..324: note = Synthetic: VL DNA
  source 1..324: mol_type = other DNA
gaaattgtgttgacgcagtctccaggcaccctgtctttgtctccaggggaaagagccaccctctcctgcaggaccagtcagagtattcagggcatcttgttagcctggtaccagcagaaacctggccaggctcccaggctcctcatctatggtgcatccagcagggccactggcatccccgacaggttcagtggcagtgggtctgggacagacttcactctcagcatcagcagactggagcctgaagattttgcagtgtattactgtcagcagtatggcagttcacctcggacgttcggccaagggaccaaggtggaaatcaaa

SEQ ID NO: 23 - protein, 12 aa, synthetic construct
  REGION 1..12: note = Synthetic: LCDR1 (Kabat)
RTSQSINAILLA

SEQ ID NO: 24 - protein, 108 aa, synthetic construct
  REGION 1..108: note = Synthetic: VL AA
EIVLTQSPGTLSLSPGERATLSCRTSQSINAILLAWYQQKPGQAPRLLIYGASSRATGIPDRFSGSGSGTDFTLSISRLEPEDFAVYYCQQYGSSPRTFGQGTKVEIK

SEQ ID NO: 25 - DNA, 324 bp, synthetic construct
  misc_feature 1..324: note = Synthetic: VL DNA
  source 1..324: mol_type = other DNA
gaaattgtgttgacgcagtctccaggcaccctgtctttgtctccaggggaaagagccaccctctcctgcaggaccagtcagagtattaacgccatcttgttagcctggtaccagcagaaacctggccaggctcccaggctcctcatctatggtgcatccagcagggccactggcatccccgacaggttcagtggcagtgggtctgggacagacttcactctcagcatcagcagactggagcctgaagattttgcagtgtattactgtcagcagtatggcagttcacctcggacgttcggccaagggaccaaggtggaaatcaaa

SEQ ID NO: 26 - protein, 116 aa, synthetic construct
  REGION 1..116: note = Synthetic: VH AA
QVQLQESGPGLVKPSGTLSLTCAVSGGSISTSNYWNWVRQSPGKGLEWIGEIYPTGNTNYSPSLKSRVTISIDKSKNQFSLKLNSVTAADTAVYYCARGYNWFDPWGQGTLVTVSS

SEQ ID NO: 27 - DNA, 348 bp, synthetic construct
  misc_feature 1..348: note = Synthetic: VH DNA
  source 1..348: mol_type = other DNA
caggtgcagctgcaggagtcgggcccaggactggtgaagccttcggggaccctgtccctcacctgcgctgtctctggtggctccatcagtactagtaactactggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatacaactggttcgacccctggggccagggaaccctggtcaccgtctcctca

SEQ ID NO: 28 - protein, 7 aa, synthetic construct
  REGION 1..7: note = Synthetic: HCDR3 (Kabat)
GYNYFDP

SEQ ID NO: 29 - protein, 116 aa, synthetic construct
  REGION 1..116: note = Synthetic: VH AA
QVQLQESGPGLVKPSGTLSLTCAVSGGSISTSNYWNWVRQSPGKGLEWIGEIYPTGNTNYSPSLKSRVTISIDKSKNQFSLKLNSVTAADTAVYYCARGYNYFDPWGQGTLVTVSS

SEQ ID NO: 30 - DNA, 348 bp, synthetic construct
  misc_feature 1..348: note = Synthetic: VH DNA
  source 1..348: mol_type = other DNA
caggtgcagctgcaggagtcgggcccaggactggtgaagccttcggggaccctgtccctcacctgcgctgtctctggtggctccatcagtactagtaactactggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatacaactacttcgacccctggggccagggaaccctggtcaccgtctcctca

SEQ ID NO: 31 - protein, 329 aa, synthetic construct
  REGION 1..329: note = Synthetic: IgG1mf
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - skipped

SEQ ID NO: 33 - protein, 330 aa, synthetic construct [sequence omitted]

SEQ ID NO: 34 - protein, 107 aa, synthetic construct [sequence omitted]

SEQ ID NO: 35-77 - skipped (43 entries)

SEQ ID NO: 78 - protein, 118 aa, synthetic construct
  REGION 1..118: note = Synthetic: VH of Tislelizumab
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 79 - protein, 107 aa, synthetic construct
  REGION 1..107: note = Synthetic: VL of Tislelizumab
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 80 - protein, 213 aa, synthetic construct
  REGION 1..213: note = Synthetic: Light Chain Sequence
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 452 aa, synthetic construct
  REGION 1..452: note = Synthetic: Heavy Chain Sequence
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82-299 - skipped (218 entries)

SEQ ID NO: 300 - protein, 5 aa, synthetic construct
  REGION 1..5: note = Synthetic: HCDR1 (Kabat)
NYDIN

SEQ ID NO: 301-399 - skipped (99 entries)

SEQ ID NO: 400 - protein, 17 aa, synthetic construct
  REGION 1..17: note = Synthetic: HCDR2 (Kabat)
WIYPGDGSAYYNEKFKG

SEQ ID NO: 401-499 - skipped (99 entries)

SEQ ID NO: 500 - protein, 10 aa, synthetic construct
  REGION 1..10: note = Synthetic: HCDR3 (Kabat)
WGYGGVPFAY

SEQ ID NO: 501-599 - skipped (99 entries)

SEQ ID NO: 600 - protein, 17 aa, synthetic construct
  REGION 1..17: note = Synthetic: LCDR1 (Kabat)
KSSQSLLNDINQKNYLA

SEQ ID NO: 601-699 - skipped (99 entries)

SEQ ID NO: 700 - protein, 7 aa, synthetic construct
  REGION 1..7: note = Synthetic: LCDR2 (Kabat)
FASTGVS

SEQ ID NO: 701-799 - skipped (99 entries)

SEQ ID NO: 800 - protein, 9 aa, synthetic construct
  REGION 1..9: note = Synthetic: LCDR3 (Kabat)
QQYYSFPWT

SEQ ID NO: 801 - protein, 534 aa, Homo sapiens
  REGION 1..534: note = misc_feature - Full-length human B7H3
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 802-899 - skipped (98 entries)

SEQ ID NO: 900 - protein, 119 aa, synthetic construct
  REGION 1..119: note = Synthetic: Antibody BGA-3295, VH AA
QIQVVQSGPELVKPGSLVKISCKASGYTFTNYDINWVKQRPGQGLEWIGWIYPGDGSAYYNEKFKGKATLTADKSSSTAYMQLSSLTSENSAVYFCARWGYGGVPFAYWGQGTLVTVSA

SEQ ID NO: 901-999 - skipped (99 entries)

SEQ ID NO: 1000 - protein, 113 aa, synthetic construct
  REGION 1..113: note = Synthetic: Antibody BGA-3295, VL AA
DIVMTQSPSSLAMSVGQKVTMSCKSSQSLLNDINQKNYLAWYQQKPGQSPKLLVYFASTGVSGVPDRFIGSGSGTDFTLTVSSVQAEDLADYFCQQYYSFPWTFGGGTKLEIK

SEQ ID NO: 1001-1099 - skipped (99 entries)

SEQ ID NO: 1100 - DNA, 357 bp, synthetic construct
  misc_feature 1..357: note = Synthetic: Antibody BGA-3295, VH DNA
  source 1..357: mol_type = other DNA
cagatccaagttgtgcagtctggacctgagctggtgaagcctgggagtttagtgaagatatcctgcaaggcttctggttacaccttcacaaactacgatataaactgggtgaagcagaggcctggacagggacttgagtggattggatggatttatcctggagatggtagtgcttactacaatgagaagttcaagggcaaggccacactgactgcagacaaatcctccagcacagcctacatgcagctcagcagtctgacttctgagaactctgcagtctatttctgtgcaagatggggttacgggggggtcccgtttgcttactggggccaagggactctggtcactgtctctgca

SEQ ID NO: 1101-1199 - skipped (99 entries)

SEQ ID NO: 1200 - DNA, 339 bp, synthetic construct
  misc_feature 1..339: note = Synthetic: Antibody BGA-3295, VL DNA
  source 1..339: mol_type = other DNA
gacattgtgatgacacagtctccatcctccctggctatgtcagtaggacagaaggtcactatgagctgcaagtccagtcagagccttttaaatgatatcaatcaaaagaactatttggcctggtaccagcagaaaccaggacagtctcctaaacttcttgtatactttgcatccactggggtatctggggtccctgatcgcttcataggcagtggatctgggacagatttcactcttaccgtcagcagtgtgcaggctgaagacctggcagattacttctgtcagcaatattatagttttccgtggacgttcggtggaggcaccaagctagaaatcaaa

SEQ ID NO: 1201-1299 - skipped (99 entries)

SEQ ID NO: 1300 - protein, 119 aa, synthetic construct
  REGION 1..119: note = Synthetic: BGA-4348, VH AA
QIQLVQSGAEVKKPGSSVKVSCKASGYTFTNYDINWVRQAPGQGLEWMGWIYPGDGSAYYNEKFKGRATITADKSTSTAYMELSSLRSEDTAVYYCARWGYGGVPFAYWGQGTLVTVSS

SEQ ID NO: 1301-1399 - skipped (99 entries)

SEQ ID NO: 1400 - protein, 113 aa, synthetic construct
  REGION 1..113: note = Synthetic: BGA-4348, VL AA
DIVMTQSPDSLAVSLGERATINCKSSQSLLNDINQKNYLAWYQQKPGQSPKLLVYFASTGVSGVPDRFSGSGSGTDFTLTISSLQAEDVAVYYCQQYYSFPWTFGGGTKVEIK

SEQ ID NO: 1401-1499 - skipped (99 entries)

SEQ ID NO: 1500 - DNA, 357 bp, synthetic construct
  misc_feature 1..357: note = Synthetic: BGA-4348, VH DNA
  source 1..357: mol_type = other DNA
cagattcagctggtgcagagcggcgccgaggtgaagaaacccggctcctccgtgaaagtcagctgcaaggccagcggctacaccttcaccaactacgacatcaactgggtgaggcaagctcccggccaaggactggagtggatgggatggatctaccccggcgacggcagcgcctactacaacgagaagttcaagggaagagccaccatcaccgccgacaagagcaccagcacagcctacatggagctgagctctctgaggtccgaggacaccgccgtgtactattgcgccagatggggctatggcggcgtgcctttcgcctattggggccaaggcacactggtgacagtgagcagc

SEQ ID NO: 1501-1599 - skipped (99 entries)

SEQ ID NO: 1600 - DNA, 339 bp, synthetic construct
  misc_feature 1..339: note = Synthetic: BGA-4348, VL DNA
  source 1..339: mol_type = other DNA
gacatcgtgatgacccagagccccgactctctggctgtgtctctgggcgagagagccaccatcaactgcaagagcagccagtctctgctgaacgacatcaaccagaagaactatctggcttggtaccagcagaagcccggccagagccctaagctgctggtgtactttgccagcaccggcgtcagcggagtgcccgataggttttccggcagcggctccggcacagactttacactgaccatctcctctctgcaagccgaggacgtggctgtgtactactgccagcagtactactccttcccttggaccttcggcggcggcacaaaggtggagattaag

SEQ ID NO: 1601-1699 - skipped (99 entries)

SEQ ID NO: 1700 - protein, 17 aa, synthetic construct
  REGION 1..17: note = Synthetic: BGA-5063, HCDR2 (Kabat)
WIYPGDASAYYNEKFKG

SEQ ID NO: 1701-1799 - skipped (99 entries)

SEQ ID NO: 1800 - protein, 119 aa, synthetic construct
  REGION 1..119: note = Synthetic: BGA-5063, VH AA
QVQLVQSGAEVKKPGSSVKVSCKASGYTFTNYDINWVRQAPGQGLEWMGWIYPGDASAYYNEKFKGRATITADKSTSTAYMELSSLRSEDTAVYYCARWGYGGVPFAYWGQGTLVTVSS

SEQ ID NO: 1801-1899 - skipped (99 entries)

SEQ ID NO: 1900 - DNA, 357 bp, synthetic construct
  misc_feature 1..357: note = Synthetic: BGA-5063, VH DNA
  source 1..357: mol_type = other DNA
caggtgcagctggtgcagagcggcgccgaggtgaagaaacccggctcctccgtgaaagtcagctgcaaggccagcggctacaccttcaccaactacgacatcaactgggtgaggcaagctcccggccaaggactggagtggatgggatggatctaccccggcgacgccagcgcctactacaacgagaagttcaagggaagagccaccatcaccgccgacaagagcaccagcacagcctacatggagctgagctctctgaggtccgaggacaccgccgtgtactattgcgccagatggggctatggcggcgtgcctttcgcctattggggccaaggcacactggtgacagtgagcagc